(19) United States
(12) Patent Application Publication
(10) Pub. No.: US 2026/0209193 A1
(43) Pub. Date: Jul. 23, 2026

(54) NOVEL INHIBITORS OF DNA DAMAGE RESPONSES FOR CANCER THERAPY

(71) Applicant: The Regents of the University of California, Oakland, CA (US)

(72) Inventors: Michael E. Jung, Los Angeles, CA (US); Cun Yu Wang, Los Angeles, CA (US); Ping Zhou, Los Angeles, CA (US); Bijay Shrestha, Los Angeles, CA (US)

(21) Appl. No.: 19/140,772

(22) PCT Filed: Dec. 29, 2023
(86) PCT No.: PCT/US2023/086435

§ 371 Date: Jun. 18, 2025

Related U.S. Application Data
(60) Provisional application No. 63/436,316, filed on Dec. 30, 2022.

(51) Int. Cl. C07D 265/30; A61K 31/166; A61K 31/17; A61K 31/27; A61K 31/337; A61K 31/341; A61K 31/404; A61K 31/407; A61K 31/416; A61K 31/4192; A61K 31/421; A61K 31/437; A61K 31/438; A61K 31/4535; A61K 31/454; A61K 31/4545; A61K 31/4965; A61K 31/4985; A61K 31/506; A61K 31/5375; A61K 31/55; A61P 35/00; C07C 235/54; C07C 271/16; C07C 275/40; C07D 203/12; C07D 207/06; C07D 207/09; C07D 209/38; C07D 209/52; C07D 211/60; C07D 223/04; C07D 231/56; C07D 241/28; C07D 249/04; C07D 263/06; C07D 295/088; C07D 307/14; C07D 401/04; C07D 401/12; C07D 403/06; C07D 403/12; C07D 409/06; C07D 471/04; C07D 491/056; C07D 491/107

(52) U.S. Cl. CPC C07D 265/30; A61K 31/166; A61K 31/17; A61K 31/27; A61K 31/337; A61K 31/341; A61K 31/404; A61K 31/407; A61K 31/416; A61K 31/4192; A61K 31/421; A61K 31/437; A61K 31/438; A61K 31/4535; A61K 31/454; A61K 31/4545; A61K 31/4965; A61K 31/4985; A61K 31/506; A61K 31/5375; A61K 31/55; A61P 35/00; C07C 235/54; C07C 271/16; C07C 275/40; C07D 203/12; C07D 207/06; C07D 207/09; C07D 209/38; C07D 209/52; C07D 211/60; C07D 223/04; C07D 231/56; C07D 241/28; C07D 249/04; C07D 263/06; C07D 295/088; C07D 307/14; C07D 401/04; C07D 401/12; C07D 403/06; C07D 403/12; C07D 409/06; C07D 471/04; C07D 491/056; C07D 491/107

(57) ABSTRACT

Disclosed herein are compounds, compositions, and methods for treating cancer and/or the repair of DNA.

### RELATED APPLICATIONS

This application claims the benefit of priority to U.S. Provisional Application No. 63/436,316, filed Dec. 30, 2022; the entire contents of which are incorporated herein by reference.

### STATEMENT OF GOVERNMENT SUPPORT

This invention was made with government support under Grant Number DE030445, awarded by the National Institutes of Health. The government has certain rights in the invention.

### BACKGROUND

PARP1 and PARP2 are two key enzymes that mediate DNA damage responses (DDR) by serving as DNA damage sensors and designal transducers. To respond to DNA damage, such as nicks and double-strand breaks (DSB), PARP1 is rapidly recruited to the sites of the damaged DNA, and its catalytic activity increases 10- to 500-fold through an allosteric activation mechanism. This results in the synthesis of protein-conjugated poly(ADP-ribose) (PAR) chains using NAD⁺ as a critical substrate. The negatively charged PARP functions as a high density protein binding scaffold and recruits components of the DNA damage repair machinery. Due to the rapid cellular division that is inherent to their nature, cancer cells are especially vulnerable to the inhibition of DDR mechanisms.

PARP inhibitors (PARPi) have been used to successful treat breast and ovarian cancers with BRCA gene mutations, using synthetic lethal screening. Furthermore, PARPis have also been used to treat cancer patients with homologous recombination defects. However, acquired resistance to PARPi treatments impedes optimal clinical outcome. PARPi resistance may be obtained through increased expression or activity of replication fork stabilizers. For example, in PARPi-resistant cancer cells, the ataxia-telangiectasia-mutated-and-Rad3-related kinase (ATR)/checkpoint kinase 1 (CHK1) pathway is often upregulated, thereby inducing the phosphorylation of multiple proteins that stabilize the replication fork and HR repair. Moreover, common chemotherapeutic drugs, such as cisplatin, induce drug resistance through activating DNA damage response. To overcome such resistance, patients can be treated with a combination of PARPi and ATR inhibitors.

Therefore, dual inhibitors of PARP and ATR are thus an attractive target for cancer therapy.

### SUMMARY OF THE INVENTION

In one aspect, the present disclosure provides compounds having a structure represented by Formula I, Formula II, or Formula III, or a pharmaceutically acceptable salt thereof:

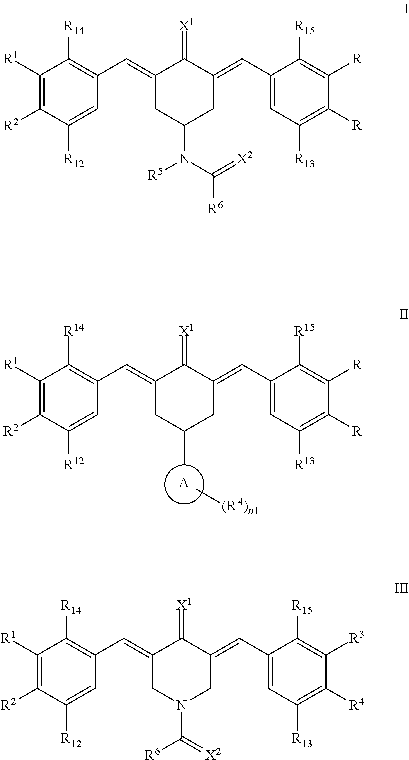

wherein:
A is aryl or heteroaryl;
n1 is 1, 2, 3, 4, or 5;
X¹ is O, S, or NR;
X² is O, S, or NR⁸;
each of R², R⁴, R¹², R¹³, R¹⁴, and R¹⁵ is independently selected from H, fluoro, chloro, bromo, and iodo;
each of R¹ and R³ is independently selected from H, fluoro, chloro, bromo, iodo, and —O(alkyl);
each of R⁵, R⁷, and R⁸ is independently selected from H, alkyl and aralkyl;
R^{A} is alkyl, acyl, or amido;
R^{C} is H, alkyl, or acyl; and
R⁶ is alkyl, aryl, heteroaryl, or heterocyclyl.

In another aspect, the present disclosure provides a pharmaceutical composition, comprising a compound disclosed herein and a pharmaceutically acceptable excipient.

In yet another aspect, the present disclosure provides methods of treating cancer in a subject in need thereof, comprising administering to the subject a compound disclosed herein or a pharmaceutically acceptable salt thereof.

In yet another aspect, the present disclosure provides methods of inhibiting repair of DNA in a subject in need thereof, comprising administering to the subject a compound disclosed herein or a pharmaceutically acceptable salt thereof.

### BRIEF DESCRIPTION OF THE DRAWINGS

FIG. 1A-1B show that JC081 and JC099 bind to PARP1. FIG. 1A shows the SPR response curves of JC081. FIG. 1B shows the SPR response curves of JC099.

FIG. 2 shows that JC099 inhibits PARP1, but not PARP2, catalytic activity.

FIG. 3A-3D show that JC081 and JC099 trap PARP1, but not PARP2 on chromatin/DNA lesions. FIG. 3A shows the chromatin fractionation assays of PARP1 for breast cancer SUM149PT-BRCA1ₘᵤₜ and SUM149PT-BRCA1ᵣₑᵥ cells in the presence of JC099, Olaparib (OLA), or Talazoparib (TALA) and 0.01% MMS. FIG. 3B shows chromatin fractionation assays of PARP1 for ovarian cancer UWB1.289 and UWB1.289+BRCA1 cells in the presence of JC099 or TALA and 0.01% MMS. FIG. 3C shows chromatin fractionation assays of PARP1 for prostate cancer C4-2B/MR and PC3 cells in the presence of JC099, JC081, or OLA and 0.01% MMS. FIG. 3D shows chromatin fractionation assays of PARP2 for breast cancer SUM149PT-BRCA1ₘᵤₜ and SUM149PT-BRCA1ᵣₑᵥ cells in the presence of JC099, OLA, or TALA and 0.01% MMS.

FIG. 4A-4B show that JC081 and JC099 inhibit HR. FIG. 4A shows qPCR data of HR assay in the presence of increased JC081 and JC099 in the prostate cancer cell line PC3. FIG. 4B shows qPCR data of HR assay in the presence of increasing concentrations of JC099 in breast cancer cell line SUM149PT-BRCA1ᵣₑᵥ.

FIG. 5A-5B show that JC099 inhibits ATR activity. FIG. 5A shows that JC099 inhibited ATR activity in SUM149PT-BRCA1ᵣₑᵥ cells. FIG. 5B shows the IC₅₀ of JC099 inhibiting ATR activity by a cell-independent assays.

FIG. 6A-6C show that JC081 and JC099 inhibit ATR-mediated signaling pathway. FIG. 6A shows that JC081 and JC099 inhibit the phosphorylation of Rad17 at Ser 645 and CHK1 at Ser 345 sites in prostate cancer cell lines C4-2B/MR and PC3. FIGS. 6B & 6C show that JC081 and JC099 inhibit the phosphorylation of Rad17 at Ser 645 and CHK1 at Ser 345 sites in breast cancer SUM149PT-BRCA1ᵣₑᵥ cells and ovarian cancer UWB1.289+BRCA1 cells, respectively.

FIG. 7A-7F show that JC081 and JC099 inhibit cell viability. FIGS. 7A-7C show that JC081 and JC099 inhibited cell viability in prostate cancer cell lines PC3, C4-2B/MR and C4-2B, respectively. FIG. 7D show that JC099 inhibited cell viability in breast cancer cell lines SUM149PT-BRCA1ₘᵤₜ, SUM149PT-BRCA1ᵣₑᵥ, MDA-MB-436, and MDA-MB-231, respectively. FIGS. 7E & 7F show that JC099 and JC081 inhibited cell viability in ovarian cancer cell lines UWB1.289 and UWB1.289+BRCA1, respectively.

FIG. 8A-8C show that JC081 and JC099 induce cell apoptosis. FIG. 8A shows that JC081 and JC099 induced cell apoptosis in prostate cancer PC3 and C4-2B/MR cells. FIG. 8B shows that JC099 induced cell apoptosis in ovarian cancer UWB1.289 and UWB1.289+BRCA1 cells.

FIG. 8C shows that JC099 induced cell apoptosis in breast cancer cell lines SUM149PT-BRCA1ₘᵤₜ, SUM149PT-BRCA1ᵣₑᵥ, MDA-MB-436, and MDA-MB-231.

FIG. 9A-9D show that JC081 and JC099 inhibit the growth of breast cancer MDA-MB-436 xenografts. FIG. 9A shows the image of tumors after the treatment of Olaparib, JC081 and JC099; FIG. 9B shows tumor weight after the treatment of Olaparib, JC081 and JC099. FIG. 9C shows tumor volume after the treatment of Olaparib, JC081 and JC099. FIG. 9D shows the body weight of mice after the treatment of Olaparib, JC081 and JC099. (One-way ANOVA, * P<0.05; ** P<0.01) FIG. 10A-10D show that JC081 and JC099 inhibit the growth of breast cancer MDA-MB-231 xenografts. FIG. 10A shows the image of tumors after the treatment of Olaparib, JC081 and JC099. FIG. 10B shows tumor weight after the treatment of Olaparib, JC081 and JC099. FIG. 10C shows tumor volume after the treatment of Olaparib, JC081 and JC099. FIG. 10D shows the body weight of mice after the treatment of Olaparib, JC081 and JC099. (One-way ANOVA, * P<0.05; ** P<0.01) FIG. 11A-11D show that JC081 and JC099 inhibit the growth of PC3 xenografts. FIG. 11A shows the image of tumors after the treatment of ENZ, Olaparib, JC081 and JC099. FIG. 1B shows tumor weight after the treatment of ENZ, Olaparib, JC081 and JC099. FIG. 11C shows tumor volume after the treatment of ENZ, Olaparib, JC081 and JC099. FIG. 11D shows the body weight of mice after the treatment of ENZ, Olaparib, JC081 and JC099. (One-way ANOVA, * P<0.05; **P<0.01) FIG. 12A-12D show that JC081 and JC099 inhibit the growth of C4-2B/MR xenografts.

FIG. 12A shows the image of tumors after the treatment of ENZ, Olaparib, JC081 and JC099. FIG. 12B shows tumor weight after the treatment of ENZ, Olaparib, JC081 and JC099. FIG. 12C shows tumor volume after the treatment of ENZ, Olaparib, JC081 and JC099. FIG. 12D shows body weight of mice after the treatment of ENZ, Olaparib, JC081 and JC099. (One-way ANOVA, *P<0.05; **P<0.01) FIG. 13A-13D show that JC099 overcomes cisplatin resistance in head and neck cancer and lung cancer. FIGS. 13A & 13B, JC099 treatment, but not OLA, significantly inhibited tumor growth of SCC1R cells in vivo. FIGS. 13C & 13D show that JC099 inhibited tumor growth of H1703R cells more potently than the combination of OLA and VE-822. (One-way ANOVA, * P<0.05; ** P<0.01).

FIG. 14A-14B shows the effect of JCS003-6 on cell viability. FIG. 14A shows cell viability after treatment with 1 μM, 2 μM, and 4 μM of JCS003 and JCS006 for 48 h in PC3 and C4-2B/MR cells. FIG. 14B shows cell viability after treatment with 1 μM, 2 μM, and 4 μM of JCS004 and JCS005 for 48 h in PC3 and SUM149PT-BRCA1ₘᵤₜ cells.

FIG. 15A-15D show the effect of JCS007-19 on cell viability. FIG. 15A shows cell viability after treatment with 0.5 μM, 1 μM, and 2 μM of JCS007 and JCS008 for 48 h in PC3 and MDA-MB-231 cells. FIG. 15B shows cell viability after treatment with 0.5 μM, 1 μM, and 2 μM of JCS009 and JCS010 for 48 h in PC3 cells. FIG. 15C shows the cell viability after treatment with 0.5 μM, 1 μM, and 2 μM of JCS011-14 for 48 h in PC3 cells. FIG. 15D shows cell viability after treatment with 0.5 μM, 1 μM, and 2 μM of JCS015-19 for 48 h in PC3 and SUM149PT-BRCA1ₘᵤₜ cells.

FIG. 16A-16I show the effect of JCSO20-71 on cell viability. FIG. 16A shows cell viability after treatment with 1 μM or 2 μM of JCSO20-27 for 48 h in PC3 and SUM149PT-BRCA1ₘᵤₜ cells. FIG. 16B shows cell viability after treatment with 1 μM or 2 μM of JCSO28-35 for 48 h in PC3 and SUM149PT-BRCA1ₘᵤₜ cells. FIG. 16C shows cell viability after treatment with 1 μM or 2 μM of JCSO36-43 for 48 h in PC3 and SUM149PT-BRCA1ₘᵤₜ cells. FIG. 16D shows cell viability after treatment with 1 μM or 2 μM of JCSO44-50 for 48 h in PC3 and SUM149PT-BRCA1ₘᵤₜ cells. FIG. 16E shows cell viability after treatment with 1 μM or 2 μM of JCS051-58 for 48 h in PC3 and SUM149PT-BRCA1ₘᵤₜ cells. FIG. 16F shows cell viability after treatment with 2 μM of JCS059-63 for 48 h in PC3 and SUM149PT-BRCA1ₘᵤₜ cells. FIG. 16G shows cell viability after treatment with 2 μM of JCS064-71 for 48 h in PC3 and SUM149PT-BRCA1ₘᵤₜ cells. FIG. 16H shows cell viability after treatment with 2 μM of JCSO72-76 for 48 h in PC3 and SUM149PT-BRCA1ₘᵤₜ cells. FIG. 16I shows cell viability after treatment with 2 μM of JCSO77-84 for 48 h in PC3 and SUM149PT-BRCA1ₘᵤₜ cells.

FIG. 17A-17D show that JCS003-8 inhibits ATR activity. FIG. 17A shows that Olaparib induced phosphorylation of CHK1 at Ser 345 sites is inhibited by 0.5 μM, 1 μM, and 2 μM of JCS003 in PC3 and SUM149PT-BRCA1ᵣₑᵥ cells. FIG. 17B shows that UV induced phosphorylation of CHK1 at Ser 345 sites is inhibited by 1 μM and 2 μM of JCS004 or JCS005 in PC3 and SUM149PT-BRCA1ᵣₑᵥ cells. FIG. 17C shows that Olaparib induced phosphorylation of Rad17 at Ser 645 and CHK1 at Ser 345 sites is inhibited by 2 μM of JCS003 or JCS006 in PC3 and cells. FIG. 17D shows that Olaparib induced phosphorylation of Rad17 at Ser 645 and CHK1 at Ser 345 sites inhibited by 1 μM and 2 μM of JCS007 or JCS008 in PC3 and MDA-MB-231cells.

FIG. 18A-18F show that JCS009-29 inhibits ATR activity in PC3 cells. FIG. 18A shows that Olaparib induced phosphorylation of Rad17 at Ser 645 and CHK1 at Ser 345 sites is inhibited by 1 μM and 2 μM of JCS009-12. FIG. 18B shows that Olaparib induced phosphorylation of Rad17 at Ser 645 and CHK1 at Ser 345 sites is inhibited by 1 μM and 2 μM of JCSO11-14. FIG. 18C shows that Olaparib induced phosphorylation of Rad17 at Ser 645 and CHK1 at Ser 345 sites inhibited is by 1 μM and 2 μM of JCS015-17. FIG. 18D shows that Olaparib induced phosphorylation of Rad17 at Ser 645 and CHK1 at Ser 345 sites inhibited is by 1 μM and 2 μM of JCS018-21. FIG. 18E shows that Olaparib induced phosphorylation of Rad17 at Ser 645 and CHK1 at Ser 345 sites is inhibited by 1 μM and 2 μM of JCSO22-25. FIG. 18F shows that Olaparib induced phosphorylation of Rad17 at Ser 645 and CHK1 at Ser 345 sites is inhibited by 1 μM and 2 μM of JCSO26-29.

FIG. 19A-19F show that JCSO30-53 inhibits ATR activity in PC3 cells. FIG. 19A shows that Olaparib induced phosphorylation of Rad17 at Ser 645 and CHK1 at Ser 345 sites is inhibited by 1 μM and 2 μM of JCSO30-33. FIG. 19B shows that Olaparib induced phosphorylation of Rad17 at Ser 645 and CHK1 at Ser 345 sites inhibited by 1 μM and 2 μM of JCSO34-37. FIG. 19C shows that Olaparib induced phosphorylation of Rad17 at Ser 645 and CHK1 at Ser 345 sites is inhibited by 1 μM and 2 μM of JCSO38-41. FIG. 19D shows that Olaparib induced phosphorylation of Rad17 at Ser 645 and CHK1 at Ser 345 sites is inhibited by 1 μM and 2 μM of JCSO42-45. FIG. 19E shows that Olaparib induced phosphorylation of Rad17 at Ser 645 and CHK1 at Ser 345 sites is inhibited by 1 μM and 2 μM of JCSO46-49. FIG. 19F shows that Olaparib induced phosphorylation of Rad17 at Ser 645 and CHK1 at Ser 345 sites is inhibited by 1 μM and 2 μM of JCS050-53.

FIG. 20A-20I show that JCS054-71 inhibits ATR activity in PC3 cells. FIG. 20A shows that Olaparib induced phosphorylation of Rad17 at Ser 645 and CHK1 at Ser 345 sites is inhibited by 1 μM and 2 μM of JCS054-57. FIG. 20B shows that Olaparib induced phosphorylation of Rad17 at Ser 645 and CHK1 at Ser 345 sites is inhibited by 1 μM and 2 μM of JCS058/59/60/63.

FIG. 20C shows that Olaparib induced phosphorylation of Rad17 at Ser 645 and CHK1 at Ser 345 sites is inhibited by 1 μM and 2 μM of JCS061 or JCS062. FIG. 20D shows that Olaparib induced phosphorylation of Rad17 at Ser 645 and CHK1 at Ser 345 sites is inhibited by 1 μM and 2 μM of JCS064-67. FIG. 20E shows Olaparib induced phosphorylation of Rad17 at Ser 645 and CHK1 at Ser 345 sites is inhibited by 1 μM and 2 μM of JCS068-71. FIG. 20F shows Olaparib induced phosphorylation of Rad17 at Ser 645 and CHK1 at Ser 345 sites is inhibited by 1 μM and 2 μM of JCS072-75. FIG. 20G shows Olaparib induced phosphorylation of Rad17 at Ser 645 and CHK1 at Ser 345 sites is inhibited by 1 μM and 2 μM of JCS076-79. FIG. 20H shows Olaparib induced phosphorylation of Rad17 at Ser 645 and CHK1 at Ser 345 sites is inhibited by 1 μM and 2 μM of JCS080-83. FIG. 20I shows Olaparib induced phosphorylation of Rad17 at Ser 645 and CHK1 at Ser 345 sites is inhibited by 1 μM and 2 μM of JCS084-87.

FIG. 21A-21D show that JCS003-28 trap PARP1 on DNA lesions. FIG. 21A shows that PARP1 was trapped on DNA lesions by 0.5 μM of JCS003 or JCS006 in PC3 and C4-2B/MR cells. FIG. 21B shows that PARP1 was trapped on DNA lesions by 0.5 μM and 2 μM of JCS004 or JCS005 in PC3 and C4-2B/MR cells. FIG. 21C shows that PARP1 was trapped on DNA lesions by 0.5 μM and 2 μM of JCS008/10/11/12/13 in PC3 cells. FIG. 21D shows that PARP1 was trapped on DNA lesions by 0.5 μM and 2 μM of JCS016/23/25/27/28 in PC3 cells.

FIG. 22A-22E show that JCS030-72 trap PARP1 on DNA lesions in PC3 cells. FIG. 22A shows that PARP1 was trapped on DNA lesions by 0.5 μM of JCS16/23/25/27/28/30/31/34/36/37.

FIG. 22B shows that PARP1 was trapped on DNA lesions by 0.3 μM of JCSO27/38/41/42/43/44/46/47/48/50. FIG. 22C shows that PARP1 was trapped on DNA lesions by 0.3 μM of JCS053/54/57/58/59/60/61/62. FIG. 22D shows that PARP1 was trapped on DNA lesions by 0.3 μM of JCS063-72. FIG. 22E shows that PARP1 was trapped on DNA lesions by 0.3 μM of JCSO73-81.

FIG. 23A-23B show the effect of JC099 analogs on PARP1 trapping. FIG. 23A shows that JC064, JC081, JC099, JC103, JC113, JC117 and JC127 trapped PARP1 on DNA lesions in SUM149PT-BRCA1ᵣₑᵥ cells. FIG. 23B show JC124, JC125, JC127 and JC128 trapped PARP1 on DNA lesions in PC3 and C4-2B/MR cells.

FIG. 24A-24B show the effect of JC099 analogs on ATR inhibition. FIG. 24A shows that JC046, JC048, JC049, JC050, JC052, JC066 and JC070 inhibited OLA induced phosphorylation of Rad17 and CHK1 in PC3 and SUM149PT-BRCA1ᵣₑᵥcells, respectively. FIG. 24B show that JC046, JC124, JC125, JC127, and JC128 inhibited OLA induced phosphorylation of Rad17 and CHK1 in PC3 and SUM149PT-BRCA1ᵣₑᵥ cells, respectively.

FIG. 25A-25E show the effect of JCS085-114 on cell viability. FIG. 25A shows cell viability after treatment with 2 μM JCS085-89 for 48 h in PC-3 and SUM149PT-BRCA1ₘᵤₜ cells;

FIG. 25B shows cell viability after treatment with 1 μM or 2 μM JCS090-93 for 48 h in PC-3 and SUM149PT-BRCA1ₘᵤₜ cells; FIG. 25C shows cell viability after treatment with 1 μM or 2 μM JCS093-100 for 48 h in PC-3 and SUM149PT-BRCA1ₘᵤₜ cells; FIG. 25D shows cell viability after treatment with 1 μM or 2 μM JCS101-106 for 48 h in PC-3 and SUM149PT-BRCA1ₘᵤₜ cells;

FIG. 25E shows cell viability after treatment with 1 μM or 2 μM JCS107-114 for 48 h in PC-3 and SUM149PT-BRCA1ₘᵤₜ cells.

FIG. 26A-26. show the effect of JCS117-130 on cell viability. FIG. 26A shows cell viability after treatment with 0.5 μM or 1.5 μM JCS117-123 for 48 h in C4-2B/MR and PC-3 and cells; FIG. 26B shows cell viability after treatment with 0.5 μM or 1.5 μM JCS124-130 for 48 h in C4-2B/MR and PC-3 and cells.

FIG. 27A-27F show the effect of JCS115, 116, 131-161 on cell viability. FIG. 27A shows cell viability after treatment with 1 μM or 2 μM JCS115, 116, 131-134 for 72 h in PC-3 and SUM149PT-BRCA1ₘᵤₜ cells; FIG. 27B shows cell viability after treatment with 1 μM or 2 μM JCS135-137 for 48 h in PC-3 and SUM149PT-BRCA1ₘᵤₜ cells; FIG. 27C shows cell viability after treatment with 1 μM or 2 μM JCS138-140 for 72 h in PC-3 and SUM149PT-BRCA1ₘᵤₜ cells;

FIG. 27D shows cell viability after treatment with 1 μM or 2 μM JCS141-145 for 72 h in PC-3 and SUM149PT-BRCA1ₘᵤₜ cells; FIG. 27E shows cell viability after treatment with 1 μM or 2 μM JCS146-153 for 72 h in PC-3 and SUM149PT-BRCA1ₘᵤₜ cells; FIG. 27F shows cell viability after treatment with 1 μM or 2 μM JCS154-161 for 72 h in PC-3 and SUM149PT-BRCA1ₘᵤₜ cells.

FIG. 28A-28H show JCS088-114 inhibits ATR activity in PC3 cells. FIG. 28A shows Olaparib or UV induced phosphorylation of Rad17 at Ser 645 and CHK1 at Ser 345 sites is inhibited by 1 μM and 2 μM JCS088-89; FIG. 28B shows Olaparib or UV induced phosphorylation of Rad17 at Ser 645 and CHK1 at Ser 345 sites is inhibited by 1 μM and 2 μM JCS090-93; FIG. 28C shows Olaparib or UV induced phosphorylation of Rad17 at Ser 645 and CHK1 at Ser 345 sites is inhibited by 1 μM and 2 μM JCS094-97; FIG. 28D shows Olaparib or UV induced phosphorylation of Rad17 at Ser 645 and CHK1 at Ser 345 sites is inhibited by 1 μM and 2 μM JCS098-100; FIG. 28E shows Olaparib or UV induced phosphorylation of Rad17 at Ser 645 and CHK1 at Ser 345 sites is inhibited by 1 μM and 2 μM JCS101-104; FIG. 28F shows Olaparib or UV induced phosphorylation of Rad17 at Ser 645 and CHK1 at Ser 345 sites is inhibited by 1 μM and 2 μM JCS107-108; FIG. 28G shows Olaparib or UV induced phosphorylation of Rad17 at Ser 645 and CHK1 at Ser 345 sites is inhibited by 1 μM and 2 μM JCS105/106/109/110; FIG. 28H shows Olaparib or UV induced phosphorylation of Rad17 at Ser 645 and CHK1 at Ser 345 sites is inhibited by 1 μM and 2 μM JCS111-114.

FIG. 29A-29D show JCS117-130 inhibits ATR/ATM activity in C4-2B/MR cells. FIG. 29A shows Olaparib or UV induced phosphorylation of Rad17 at Ser 645, CHK1 at Ser 345, KAP1 at Ser 824, and CHK2 at Thr 68 sites is inhibited by 0.5 μM and 1.5 μM JCS117-118; FIG. 29B shows Olaparib or UV induced phosphorylation of Rad17 at Ser 645, CHK1 at Ser 345, KAP1 at Ser 824, and CHK2 at Thr 68 sites is inhibited by 0.5 μM and 1.5 μM JCS119-122; FIG. 29C shows Olaparib or UV induced phosphorylation of Rad17 at Ser 645, CHK1 at Ser 345, KAP1 at Ser 824, and CHK2 at Thr 68 sites is inhibited by 0.5 μM and 1.5 μM JCS123-126; FIG. 29D shows Olaparib or UV induced phosphorylation of Rad17 at Ser 645, CHK1 at Ser 345, KAP1 at Ser 824, and CHK2 at Thr 68 sites is inhibited by 0.5 μM and 1.5 μM JCS127-130.

FIG. 30A-30H show JCS115, 116, 131-160 inhibits ATR activity in PC3 cells. FIG. 30A shows Olaparib or UV induced phosphorylation of Rad17 at Ser 645 and CHK1 at Ser 345 sites is inhibited by 1 μM and 2 μM JCS115/116/131/132; FIG. 30B shows Olaparib or UV induced phosphorylation of Rad17 at Ser 645 and CHK1 at Ser 345 sites is inhibited by 1 μM and 2 μM JCS133-136; FIG. 30C shows Olaparib or UV induced phosphorylation of Rad17 at Ser 645 and CHK1 at Ser 345 sites is inhibited by 1 μM and 2 μM JCS137-140; FIG. 30D shows Olaparib induced phosphorylation of Rad17 at Ser 645 and CHK1 at Ser 345 sites is inhibited by 1 μM and 2 μM JCS141-144; FIG. 30E shows Olaparib induced phosphorylation of Rad17 at Ser 645 and CHK1 at Ser 345 sites is inhibited by 1 μM and 2 μM JCS145-148; FIG. 30F shows Olaparib induced phosphorylation of Rad17 at Ser 645 and CHK1 at Ser 345 sites is inhibited by 1 μM and 2 μM JCS149-152; FIG. 30G shows Olaparib induced phosphorylation of Rad17 at Ser 645 and CHK1 at Ser 345 sites is inhibited by 1 μM and 2 μM JCS153-156; FIG. 30H shows Olaparib induced phosphorylation of Rad17 at Ser 645 and CHK1 at Ser 345 sites is inhibited by 1 μM and 2 μM JCS157-160.

FIG. 31A-31E show JCS082-140 trap PARP1 on DNA lesions in PC3 cells. FIG. 31A shows that PARP1 was trapped on DNA lesions by 0.5 μM JCS082-089; FIG. 31B shows that PARP1 was trapped on DNA lesions by 0.5 μM JCS090-100; FIG. 31C shows that PARP1 was trapped on DNA lesions by 0.5 μM JCS101-110; FIG. 31D shows that PARP1 was trapped on DNA lesions by 0.5 μM JCS111-116; FIG. 31E shows that PARP1 was trapped on DNA lesions by 0.5 μM JCS131-140. FIG. 31F shows that PARP1 was trapped on DNA lesions by 0.3 μM JCS141-150. FIG. 31G shows that PARP1 was trapped on DNA lesions by 0.3 μM JCS151-160.

FIG. 32A-32D show JC099 analogs inhibit the growth of BRCA2-mutant breast cancer PDX. FIG. 32A shows the image of tumors after the treatment with 50 mg/kg i.g. of JC099, JCS016/25/27/41/43/63/69/86; FIG. 32B shows tumor weight after the treatment with 50 mg/kg i.g. of JC099, JCS016/25/27/41/43/63/69/86; FIG. 32C shows tumor volume after the treatment with 50 mg/kg i.g. of JC099, JCS016/25/27/41/43/63/69/86; FIG. 32D shows body weight of mice after the treatment with 50 mg/kg i.g. of JC099, JCS016/25/27/41/43/63/69/86. (One-way ANOVA, *P<0.05; **P<0.01) FIG. 33A-33D show JC099 analogs inhibit the growth of breast cancer SUM149PT-BRCA1ᵣₑᵥ xenografts. FIG. 33A shows the image of tumors after the treatment with 50 mg/kg i.g. of JC099, JCS016/25/69/86; FIG. 33B shows tumor weight after the treatment with 50 mg/kg i.g. of JC099, JCS016/25/69/86; FIG. 33C shows tumor volume after the treatment with 50 mg/kg i.g. of JC099, JCS016/25/69/86; FIG. 33D shows body weight of mice after the treatment with 50 mg/kg i.g. of JC099, JCS016/25/69/86. (One-way ANOVA, * P<0.05; *** P<0.001) FIG. 34A-34D show JC099 analogs inhibit the growth of castration-resistant prostate cancer PDX. FIG. 34A shows the image of tumors after the treatment with 50 mg/kg i.g. of JC099, JCSO25/69; FIG. 34B shows tumor weight after the treatment with 50 mg/kg i.g. of JC099, JCSO25/69; FIG. 34C shows tumor volume after the treatment with 50 mg/kg i.g. of JC099, JCSO25/69; FIG. 34D shows the body weight of mice after the treatment with 50 mg/kg i.g. of JC099, JCSO25/69. (One-way ANOVA, * P<0.05; ** P<0.01; *** P<0.001) FIG. 35A-35D show JC099 analogs inhibit the growth of Olaparib-resistant breast cancer PDX. FIG. 35A shows the image of tumors after the treatment with 60 mg/kg i.g. of JC099, JCSO90/136/140; FIG. 35B shows tumor weight after the treatment with 60 mg/kg i.g. of JC099, JCSO90/136/140; FIG. 35C shows tumor volume after the treatment with 60 mg/kg i.g. of JC099, JCSO90/136/140; FIG. 35D shows body weight of mice after the treatment with 60 mg/kg i.g. of JC099, JCSO90/136/140. (One-way ANOVA, * P<0.05; ** P<0.01; *** P<0.001)

### DETAILED DESCRIPTION OF THE INVENTION

In one aspect, the present disclosure provides compounds having a structure represented by Formula I, Formula II, or Formula III, or a pharmaceutically acceptable salt thereof:

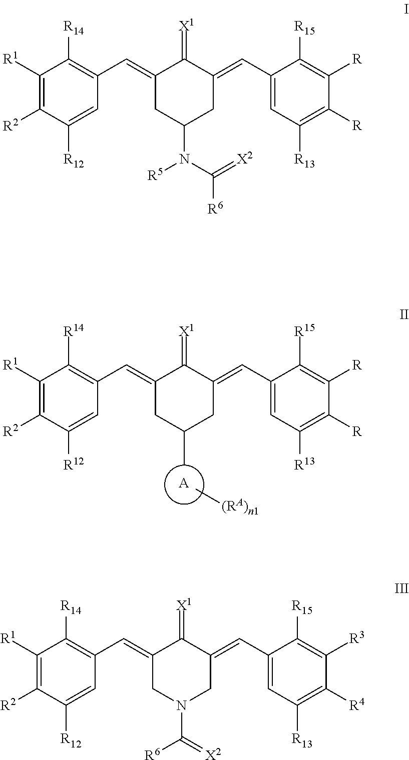

wherein:
A is aryl or heteroaryl;
n1 is 1, 2, 3, 4, or 5;
X¹ is O, S, or NR⁷;
X² is O, S, or NR;
each of R², R⁴, R¹², R¹³, R¹⁴, and R¹⁵ is independently selected from H, fluoro, chloro, bromo, and iodo;
each of R¹ and R³ is independently selected from H, fluoro, chloro, bromo, iodo, and —O(alkyl);
each of R⁵, R⁷, and R⁸ is independently selected from H, alkyl and aralkyl;
R^{A} is alkyl, acyl, or amido;
R^{C} is H, alkyl, or acyl; and
R⁶ is alkyl, aryl, heteroaryl, or heterocyclyl.

In certain embodiments, the compound has a structure represented by Formula La, or a pharmaceutically acceptable salt thereof:

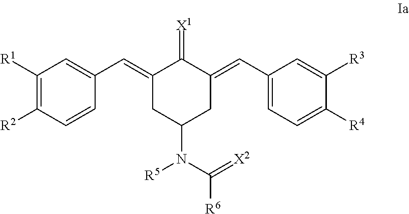

wherein:
X¹ is O, S, or NR⁷;
X² is O, S, or NR⁸;
each of R¹, R², R³, and R⁴ is independently selected from fluoro, chloro, bromo, and iodo;
each of R⁵, R⁷, and R⁸ is independently selected from H, alkyl and aralkyl; and
R⁶ is alkyl, aryl, heteroaryl, or heterocyclyl.

In certain embodiments, the compound has a structure represented by Formula I, or a

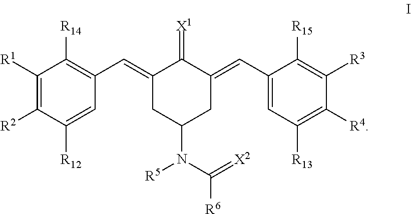

In certain embodiments, the compound has a structure represented by Formula II, or a pharmaceutically acceptable salt thereof:

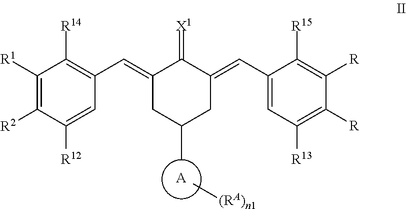

In certain embodiments, the compound has a structure represented by Formula III, or a pharmaceutically acceptable salt thereof:

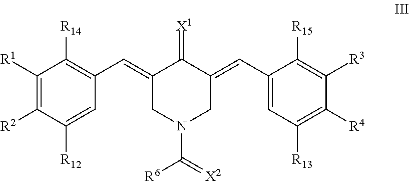

In certain embodiments, the compound is not

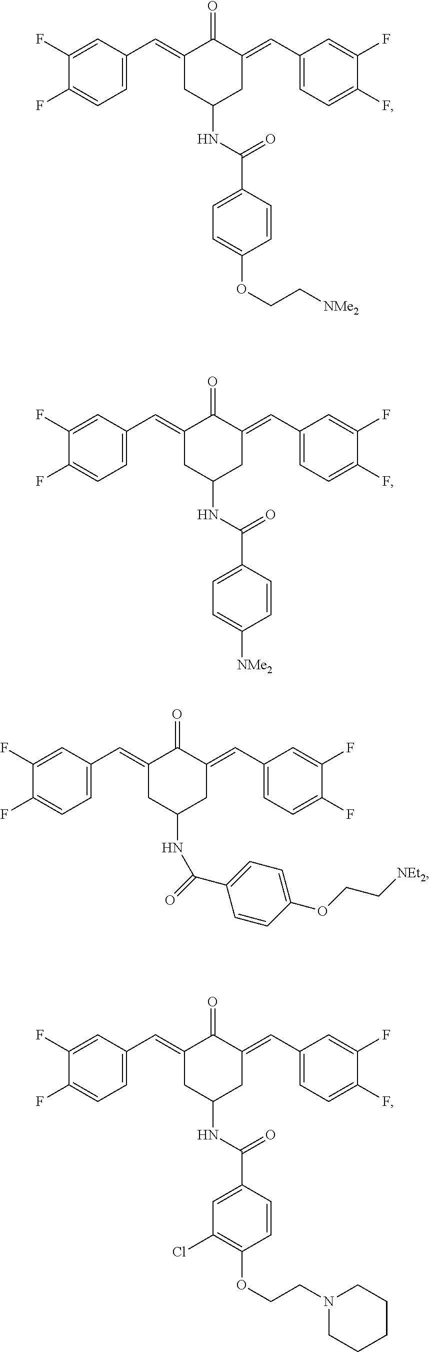

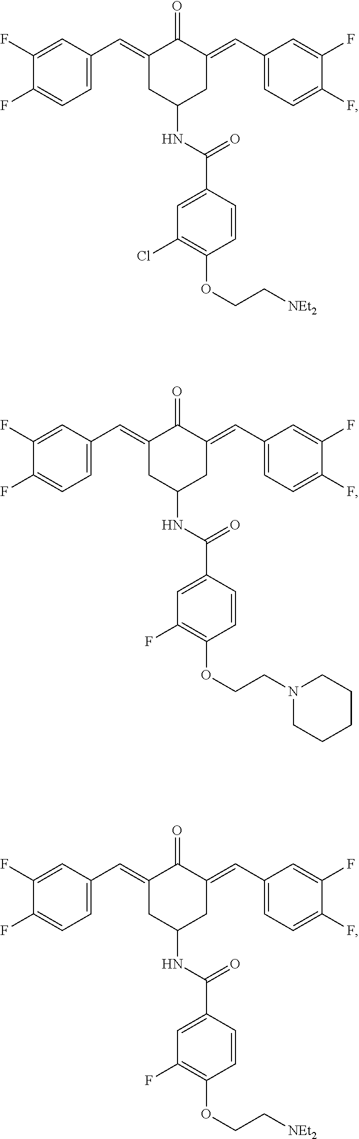

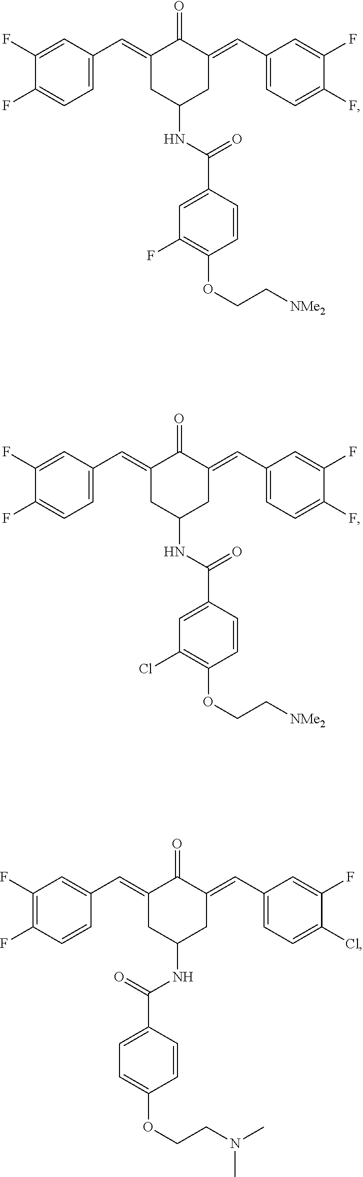 [SMILES: CN(C)CCOc1ccc(C(=O)NC2C/C(=C\c3ccc(F)c(F)c3)C(=O)/C(=C/c3ccc(F)c(F)c3)C2)cc1F ; CN(C)CCOc1ccc(C(=O)NC2C/C(=C\c3ccc(F)c(F)c3)C(=O)/C(=C/c3ccc(F)c(F)c3)C2)cc1Cl ; CN(C)CCOc1ccc(C(=O)NC2C/C(=C\c3ccc(F)c(F)c3)C(=O)/C(=C/c3ccc(Cl)c(F)c3)C2)cc1]

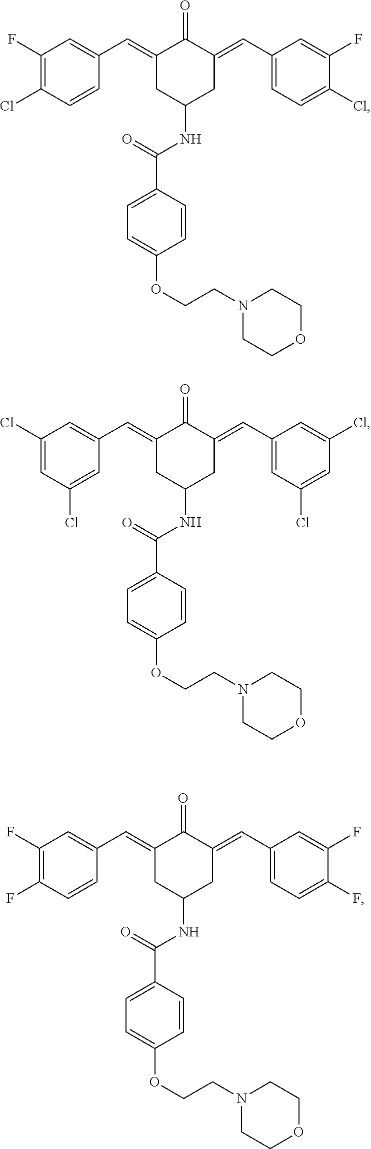 [SMILES: O=C1/C(=C/c2ccc(Cl)c(F)c2)CC(NC(=O)c2ccc(OCCN3CCOCC3)cc2)C/C1=C\c1ccc(Cl)c(F)c1 ; O=C1/C(=C/c2cc(Cl)cc(Cl)c2)CC(NC(=O)c2ccc(OCCN3CCOCC3)cc2)C/C1=C\c1cc(Cl)cc(Cl)c1 ; O=C1/C(=C/c2ccc(F)c(F)c2)CC(NC(=O)c2ccc(OCCN3CCOCC3)cc2)C/C1=C\c1ccc(F)c(F)c1]

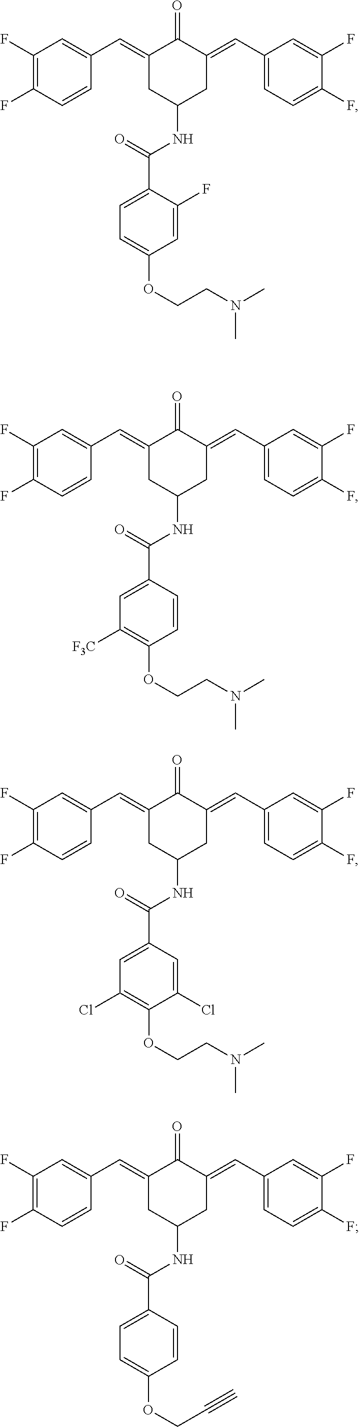 [SMILES: CN(C)CCOc1ccc(C(=O)NC2C/C(=C\c3ccc(F)c(F)c3)C(=O)/C(=C/c3ccc(F)c(F)c3)C2)c(F)c1 ; CN(C)CCOc1ccc(C(=O)NC2C/C(=C\c3ccc(F)c(F)c3)C(=O)/C(=C/c3ccc(F)c(F)c3)C2)cc1C(F)(F)F ; CN(C)CCOc1c(Cl)cc(C(=O)NC2C/C(=C\c3ccc(F)c(F)c3)C(=O)/C(=C/c3ccc(F)c(F)c3)C2)cc1Cl ; C#CCOc1ccc(C(=O)NC2C/C(=C\c3ccc(F)c(F)c3)C(=O)/C(=C/c3ccc(F)c(F)c3)C2)cc1]

or a pharmaceutically acceptable salt thereof.

In certain embodiments, the compound is not

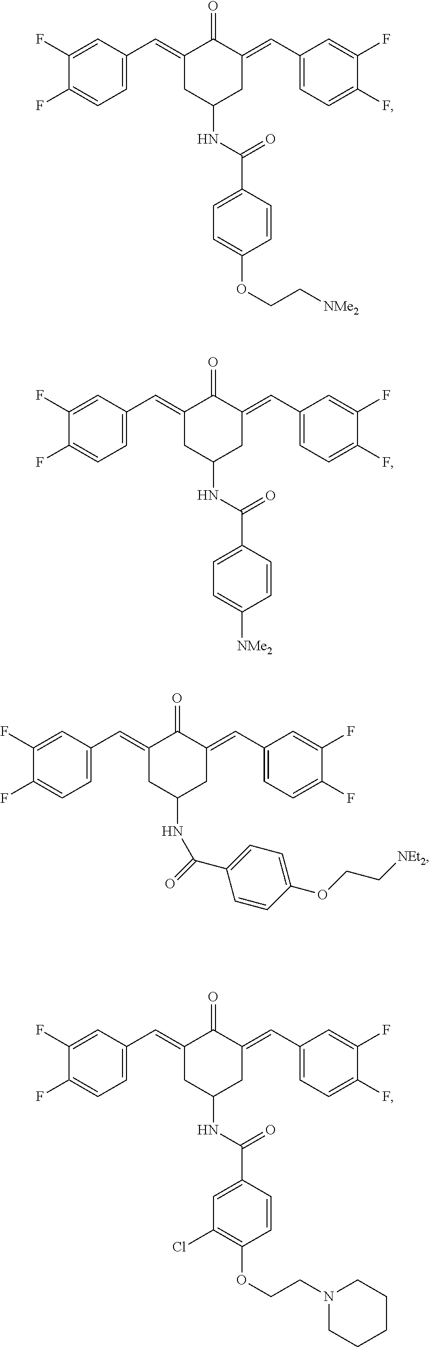

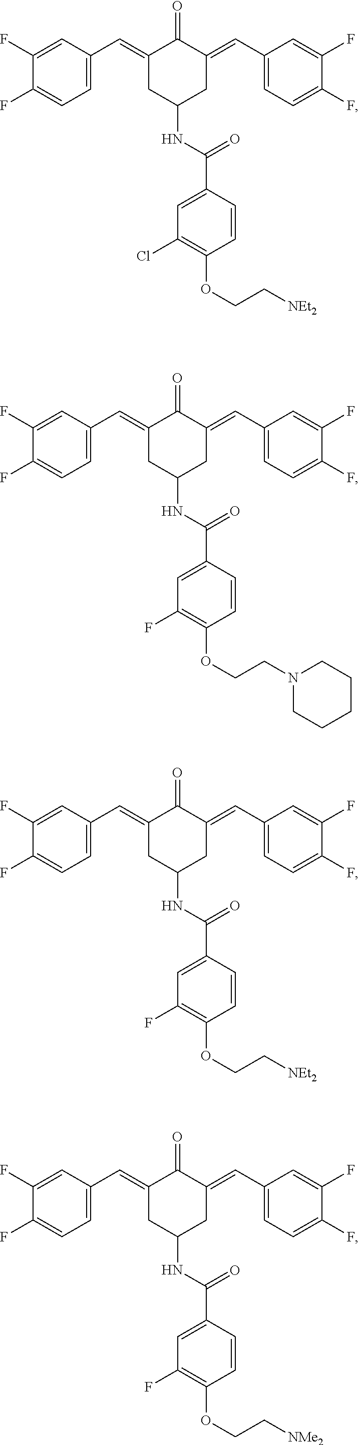

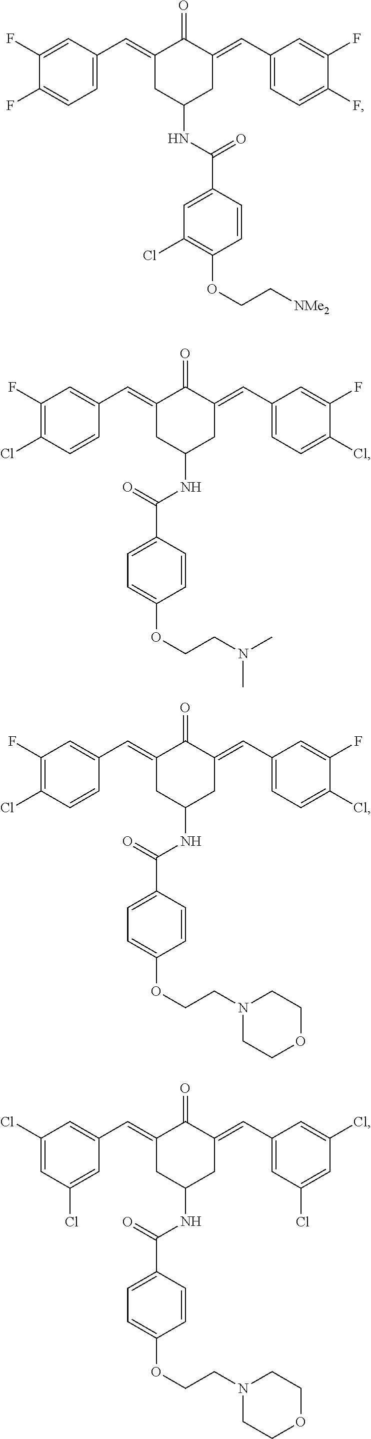 [SMILES: CN(C)CCOc1ccc(C(=O)NC2C/C(=C\c3ccc(F)c(F)c3)C(=O)/C(=C/c3ccc(F)c(F)c3)C2)cc1Cl ; CN(C)CCOc1ccc(C(=O)NC2C/C(=C\c3ccc(Cl)c(F)c3)C(=O)/C(=C/c3ccc(Cl)c(F)c3)C2)cc1 ; O=C1/C(=C/c2ccc(Cl)c(F)c2)CC(NC(=O)c2ccc(OCCN3CCOCC3)cc2)C/C1=C\c1ccc(Cl)c(F)c1 ; O=C1/C(=C/c2cc(Cl)cc(Cl)c2)CC(NC(=O)c2ccc(OCCN3CCOCC3)cc2)C/C1=C\c1cc(Cl)cc(Cl)c1]

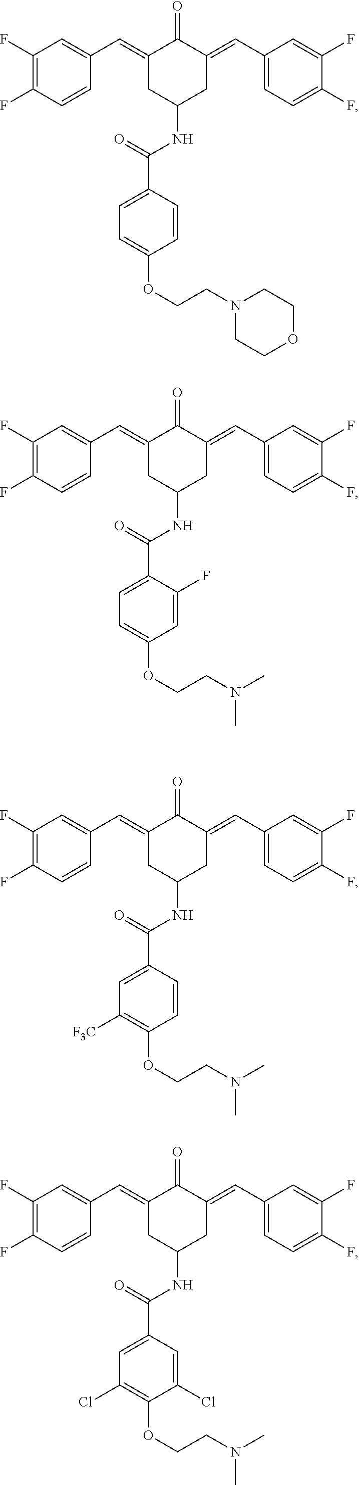 [SMILES: O=C1/C(=C/c2ccc(F)c(F)c2)CC(NC(=O)c2ccc(OCCN3CCOCC3)cc2)C/C1=C\c1ccc(F)c(F)c1 ; CN(C)CCOc1ccc(C(=O)NC2C/C(=C\c3ccc(F)c(F)c3)C(=O)/C(=C/c3ccc(F)c(F)c3)C2)c(F)c1 ; CN(C)CCOc1ccc(C(=O)NC2C/C(=C\c3ccc(F)c(F)c3)C(=O)/C(=C/c3ccc(F)c(F)c3)C2)cc1C(F)(F)F ; CN(C)CCOc1c(Cl)cc(C(=O)NC2C/C(=C\c3ccc(F)c(F)c3)C(=O)/C(=C/c3ccc(F)c(F)c3)C2)cc1Cl]

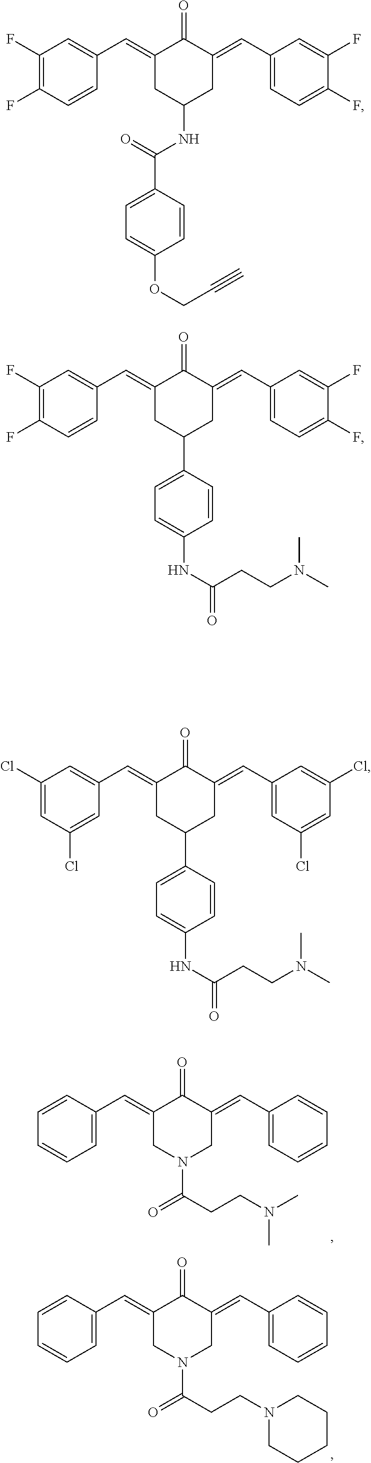

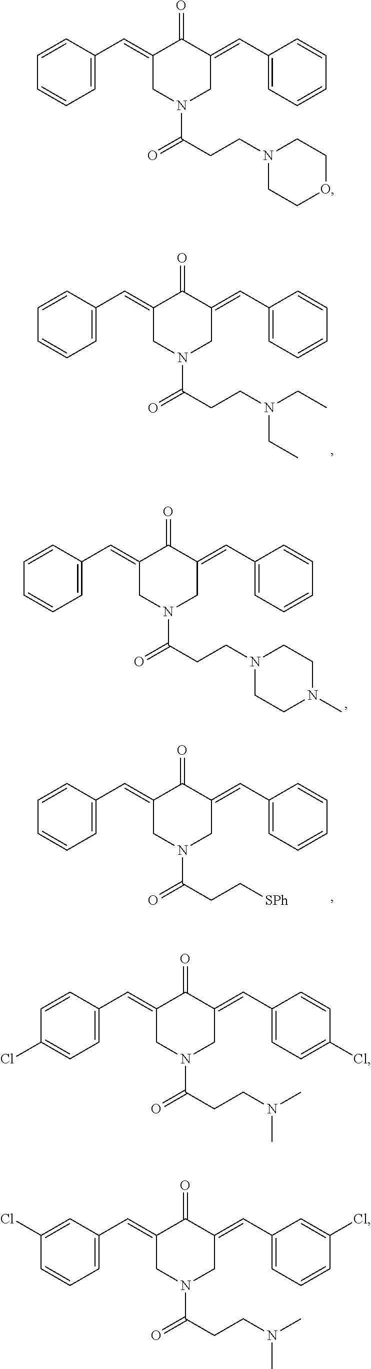

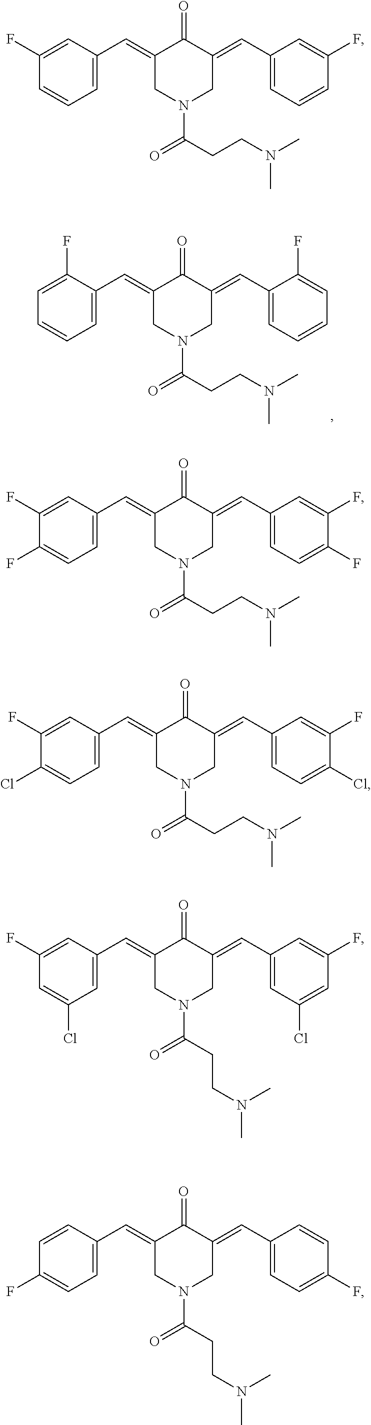 [SMILES: CN(C)CCC(=O)N1C/C(=C\c2cccc(F)c2)C(=O)/C(=C/c2cccc(F)c2)C1 ; CN(C)CCC(=O)N1C/C(=C\c2ccccc2F)C(=O)/C(=C/c2ccccc2F)C1 ; CN(C)CCC(=O)N1C/C(=C\c2ccc(F)c(F)c2)C(=O)/C(=C/c2ccc(F)c(F)c2)C1 ; CN(C)CCC(=O)N1C/C(=C\c2ccc(Cl)c(F)c2)C(=O)/C(=C/c2ccc(Cl)c(F)c2)C1 ; CN(C)CCC(=O)N1C/C(=C\c2cc(F)cc(Cl)c2)C(=O)/C(=C/c2cc(F)cc(Cl)c2)C1 ; CN(C)CCC(=O)N1C/C(=C\c2ccc(F)cc2)C(=O)/C(=C/c2ccc(F)cc2)C1]

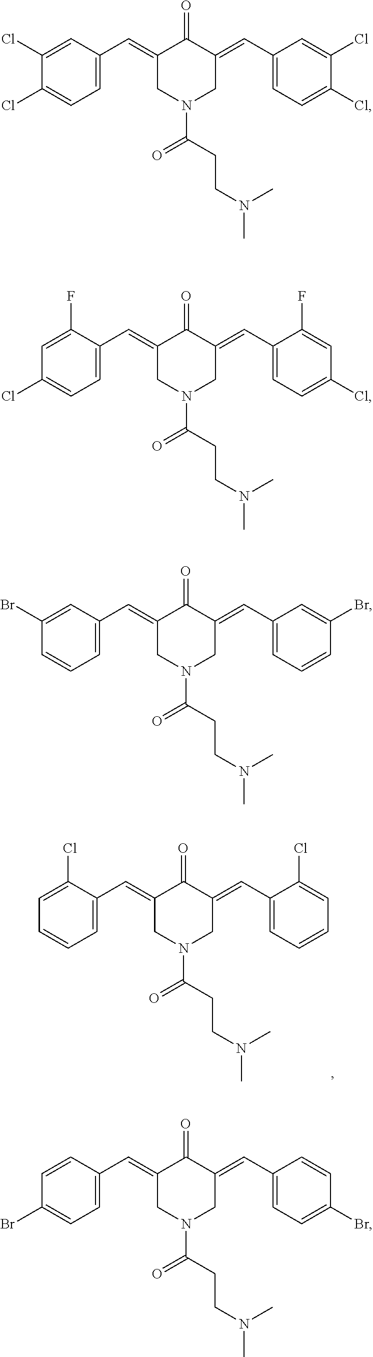 [SMILES: CN(C)CCC(=O)N1C/C(=C\c2ccc(Cl)c(Cl)c2)C(=O)/C(=C/c2ccc(Cl)c(Cl)c2)C1 ; CN(C)CCC(=O)N1C/C(=C\c2ccc(Cl)cc2F)C(=O)/C(=C/c2ccc(Cl)cc2F)C1 ; CN(C)CCC(=O)N1C/C(=C\c2cccc(Br)c2)C(=O)/C(=C/c2cccc(Br)c2)C1 ; CN(C)CCC(=O)N1C/C(=C\c2ccccc2Cl)C(=O)/C(=C/c2ccccc2Cl)C1 ; CN(C)CCC(=O)N1C/C(=C\c2ccc(Br)cc2)C(=O)/C(=C/c2ccc(Br)cc2)C1]

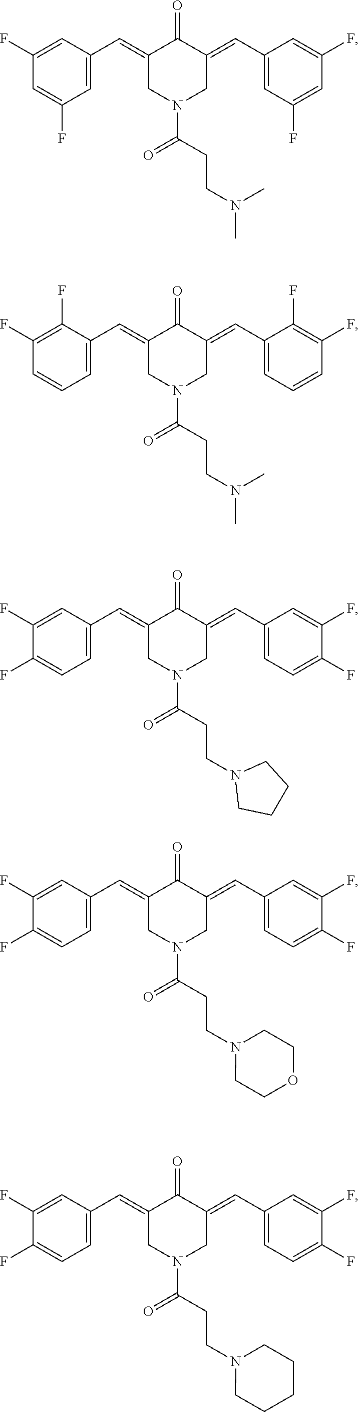 [SMILES: CN(C)CCC(=O)N1C/C(=C\c2cc(F)cc(F)c2)C(=O)/C(=C/c2cc(F)cc(F)c2)C1 ; CN(C)CCC(=O)N1C/C(=C\c2cccc(F)c2F)C(=O)/C(=C/c2cccc(F)c2F)C1 ; O=C1/C(=C/c2ccc(F)c(F)c2)CN(C(=O)CCN2CCCC2)C/C1=C\c1ccc(F)c(F)c1 ; O=C1/C(=C/c2ccc(F)c(F)c2)CN(C(=O)CCN2CCOCC2)C/C1=C\c1ccc(F)c(F)c1 ; O=C1/C(=C/c2ccc(F)c(F)c2)CN(C(=O)CCN2CCCCC2)C/C1=C\c1ccc(F)c(F)c1]

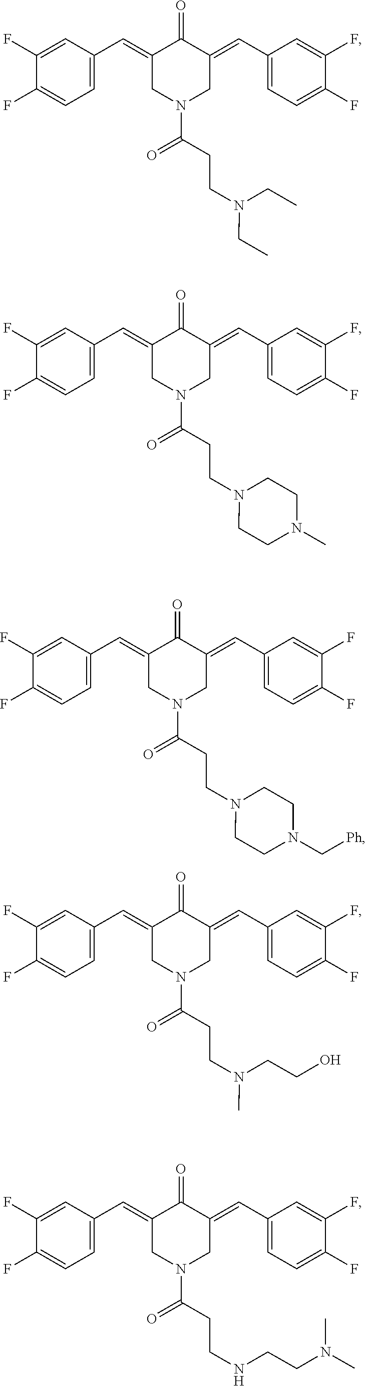 [SMILES: CCN(CC)CCC(=O)N1C/C(=C\c2ccc(F)c(F)c2)C(=O)/C(=C/c2ccc(F)c(F)c2)C1 ; CN1CCN(CCC(=O)N2C/C(=C\c3ccc(F)c(F)c3)C(=O)/C(=C/c3ccc(F)c(F)c3)C2)CC1 ; O=C1/C(=C/c2ccc(F)c(F)c2)CN(C(=O)CCN2CCN(Cc3ccccc3)CC2)C/C1=C\c1ccc(F)c(F)c1 ; CN(CCO)CCC(=O)N1C/C(=C\c2ccc(F)c(F)c2)C(=O)/C(=C/c2ccc(F)c(F)c2)C1 ; CN(C)CCNCCC(=O)N1C/C(=C\c2ccc(F)c(F)c2)C(=O)/C(=C/c2ccc(F)c(F)c2)C1]

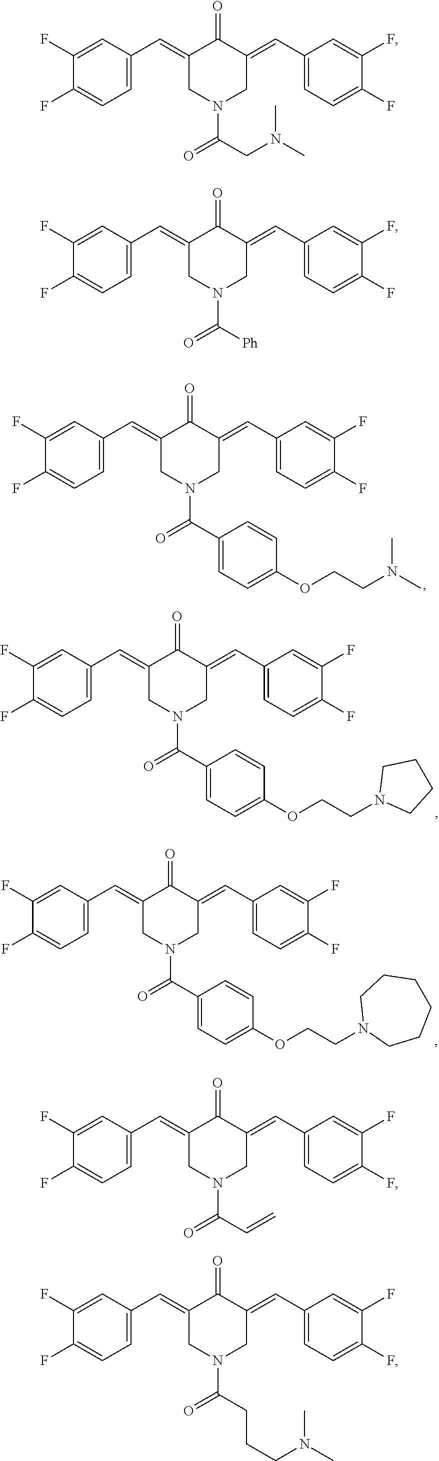

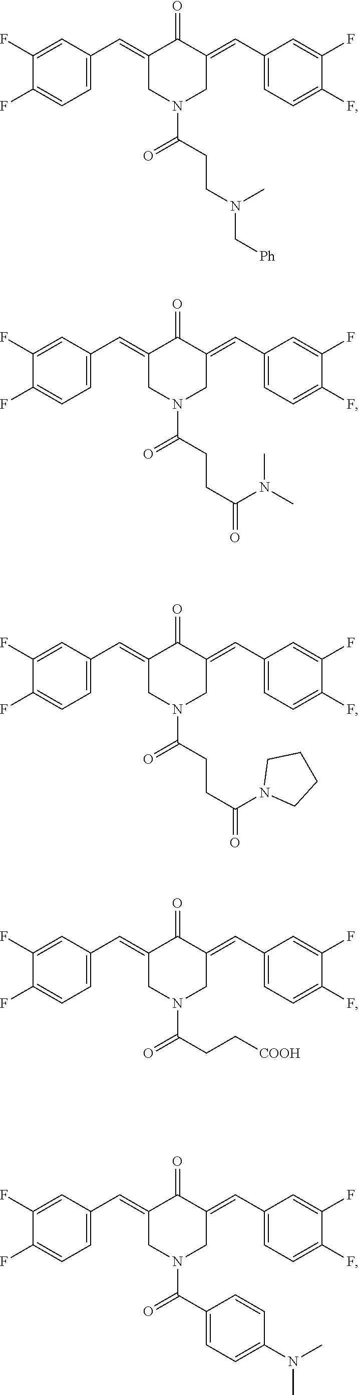 [SMILES: CN(CCC(=O)N1C/C(=C\c2ccc(F)c(F)c2)C(=O)/C(=C/c2ccc(F)c(F)c2)C1)Cc1ccccc1 ; CN(C)C(=O)CCC(=O)N1C/C(=C\c2ccc(F)c(F)c2)C(=O)/C(=C/c2ccc(F)c(F)c2)C1 ; O=C1/C(=C/c2ccc(F)c(F)c2)CN(C(=O)CCC(=O)N2CCCC2)C/C1=C\c1ccc(F)c(F)c1 ; O=C(O)CCC(=O)N1C/C(=C\c2ccc(F)c(F)c2)C(=O)/C(=C/c2ccc(F)c(F)c2)C1 ; CN(C)c1ccc(C(=O)N2C/C(=C\c3ccc(F)c(F)c3)C(=O)/C(=C/c3ccc(F)c(F)c3)C2)cc1]

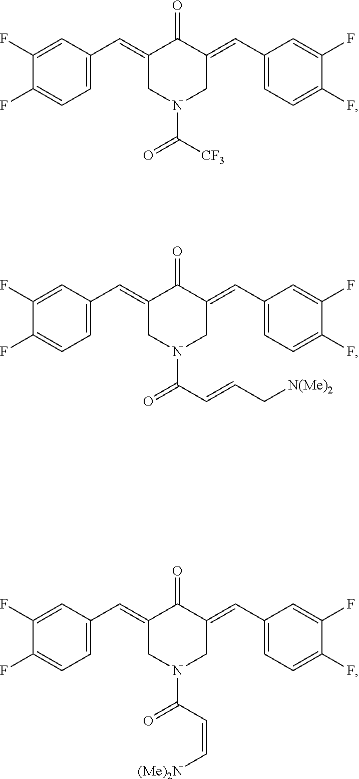

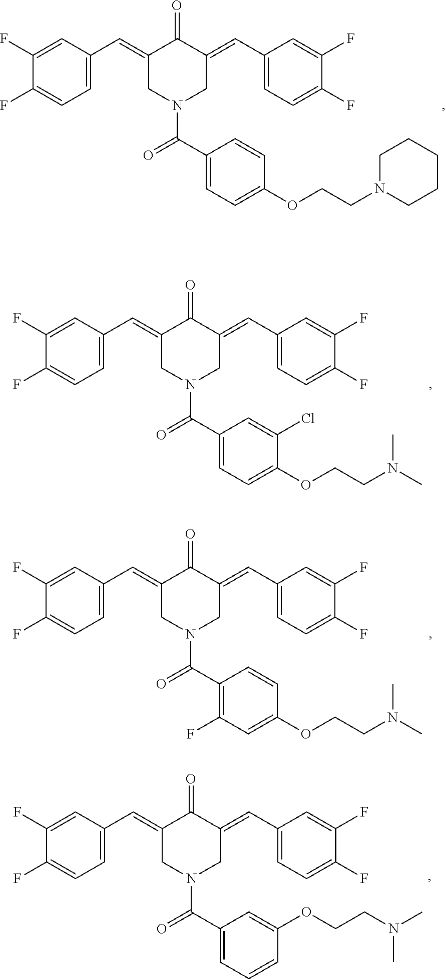

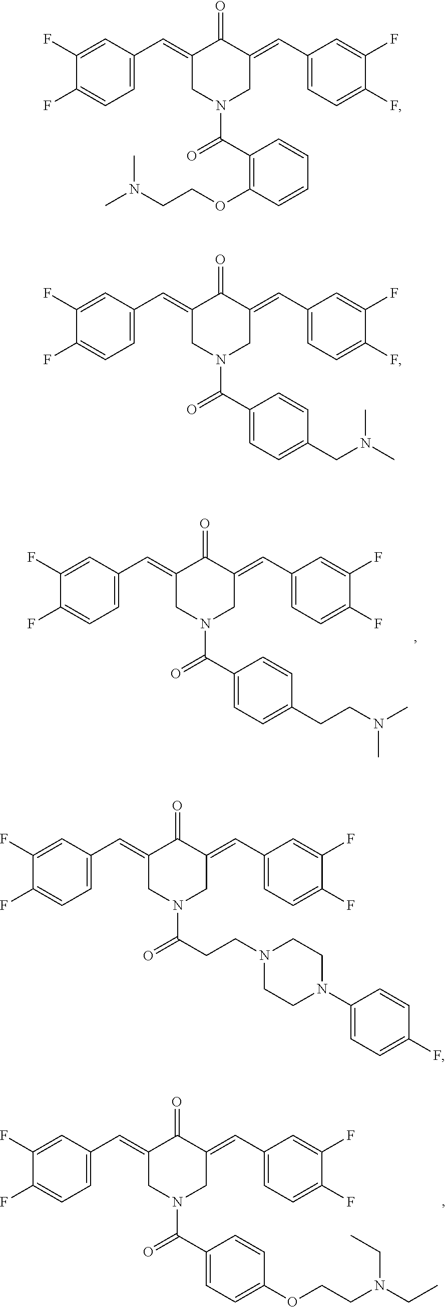 [SMILES: CN(C)CCOc1ccccc1C(=O)N1C/C(=C\c2ccc(F)c(F)c2)C(=O)/C(=C/c2ccc(F)c(F)c2)C1 ; CN(C)Cc1ccc(C(=O)N2C/C(=C\c3ccc(F)c(F)c3)C(=O)/C(=C/c3ccc(F)c(F)c3)C2)cc1 ; CN(C)CCc1ccc(C(=O)N2C/C(=C\c3ccc(F)c(F)c3)C(=O)/C(=C/c3ccc(F)c(F)c3)C2)cc1 ; O=C1/C(=C/c2ccc(F)c(F)c2)CN(C(=O)CCN2CCN(c3ccc(F)cc3)CC2)C/C1=C\c1ccc(F)c(F)c1 ; CCN(CC)CCOc1ccc(C(=O)N2C/C(=C\c3ccc(F)c(F)c3)C(=O)/C(=C/c3ccc(F)c(F)c3)C2)cc1]

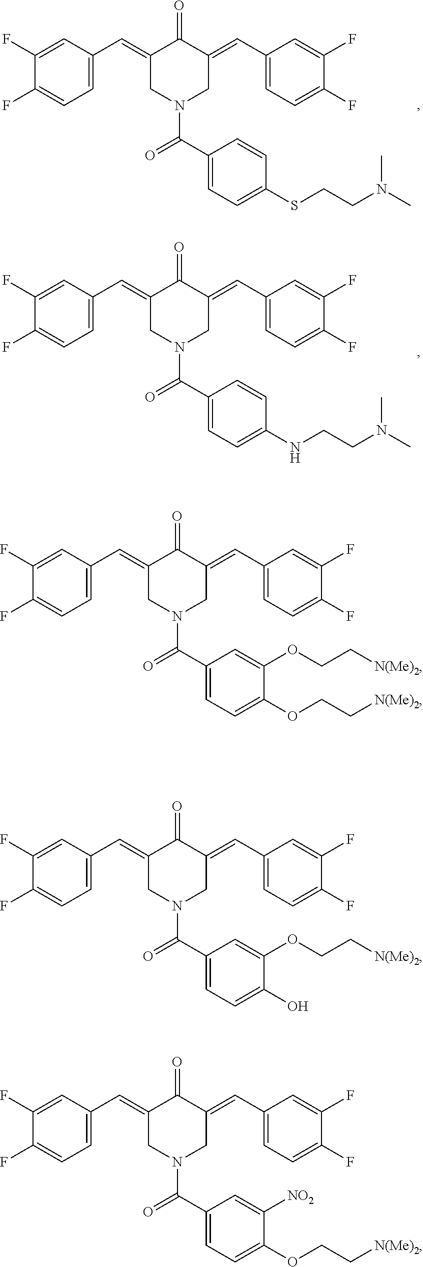

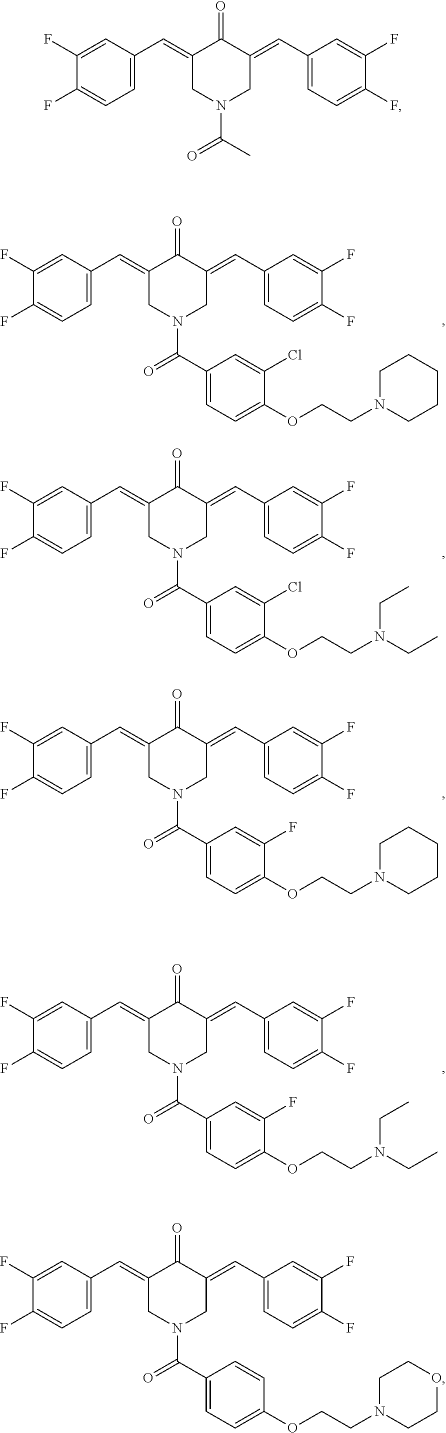 [SMILES: CC(=O)N1C/C(=C\c2ccc(F)c(F)c2)C(=O)/C(=C/c2ccc(F)c(F)c2)C1 ; O=C1/C(=C/c2ccc(F)c(F)c2)CN(C(=O)c2ccc(OCCN3CCCCC3)c(Cl)c2)C/C1=C\c1ccc(F)c(F)c1 ; CCN(CC)CCOc1ccc(C(=O)N2C/C(=C\c3ccc(F)c(F)c3)C(=O)/C(=C/c3ccc(F)c(F)c3)C2)cc1Cl ; O=C1/C(=C/c2ccc(F)c(F)c2)CN(C(=O)c2ccc(OCCN3CCCCC3)c(F)c2)C/C1=C\c1ccc(F)c(F)c1 ; CCN(CC)CCOc1ccc(C(=O)N2C/C(=C\c3ccc(F)c(F)c3)C(=O)/C(=C/c3ccc(F)c(F)c3)C2)cc1F ; O=C1/C(=C/c2ccc(F)c(F)c2)CN(C(=O)c2ccc(OCCN3CCOCC3)cc2)C/C1=C\c1ccc(F)c(F)c1]

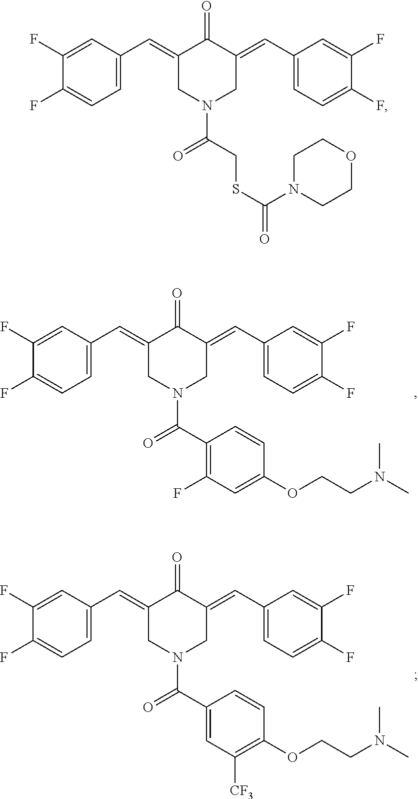 [SMILES: O=C1/C(=C/c2ccc(F)c(F)c2)CN(C(=O)CSC(=O)N2CCOCC2)C/C1=C\c1ccc(F)c(F)c1 ; CN(C)CCOc1ccc(C(=O)N2C/C(=C\c3ccc(F)c(F)c3)C(=O)/C(=C/c3ccc(F)c(F)c3)C2)c(F)c1 ; CN(C)CCOc1ccc(C(=O)N2C/C(=C\c3ccc(F)c(F)c3)C(=O)/C(=C/c3ccc(F)c(F)c3)C2)cc1C(F)(F)F]

or a pharmaceutically acceptable salt thereof.

In certain embodiments, R¹ is fluoro. In further embodiments, R¹ is chloro. In yet further embodiments, R¹ is H. In still further embodiments, R¹ is —O(alkyl) (e.g., methoxy).

In certain embodiments, R² is fluoro. In further embodiments, R² is chloro. In yet further embodiments, R² is H.

In certain embodiments, R³ is fluoro. In further embodiments, R³ is chloro. In yet further embodiments, R³ is H. In still further embodiments, R³ is —O(alkyl) (e.g., methoxy).

In certain embodiments, R⁴ is fluoro. In further embodiments, R⁴ is chloro. In yet further embodiments, R⁴ is H.

In certain embodiments, R¹² is chloro. In further embodiments, R¹² is fluoro. In yet further embodiments, R¹² is H.

In certain embodiments, R¹³ is chloro. In further embodiments, R¹³ is fluoro. In yet further embodiments, R¹³ is H.

In certain embodiments, R¹⁴ is chloro. In further embodiments, R¹⁴ is fluoro. In yet further embodiments, R¹⁴ is H.

In certain embodiments, R¹⁵ is chloro. In further embodiments, R¹⁵ is fluoro. In certain embodiments, R¹⁵ is H.

In certain embodiments, X¹ is O.

In certain embodiments, A is heteroaryl (e.g., indazolyl.)

In certain embodiments, n1 is 1.

In certain embodiments, R^{A} is alkylamido (e.g., N-diethylamido).

In certain embodiments, X² is O.

In certain embodiments, R⁵ is H.

In certain embodiments, R⁶ is alkyl. In further embodiments, R⁶ is heteroaryl (e.g., pyridinyl, pyrimidinyl, indolyl, or pyrazolopyridinyl). In yet further embodiments, R⁶ is heteroaryl (e.g., pyridinyl, pyrimidinyl, indolyl, quinazolinyl, phthalazinyl, or pyrazolopyridinyl). In still further embodiments, R⁶ is pyridinyl or pyrimidinyl. In certain embodiments, R⁶ is aryl.

In certain embodiments, R⁶ is substituted with alkyl, alkenyl, alkynyl, halo, hydroxyl, carboxyl, acyl, acetyl, ester, thioester, alkoxy, phosphoryl, amino, amido, cyano, nitro, azido, alkylthio, cycloalkyl, alkylsulfonyl, sulfonamide, cycloalkyl, aryl, heteroaryl, and heterocyclyl. In certain embodiments, R⁶ is substituted with heterocyclyl (e.g., pyrrolidinyl pyrrolidinyl, piperazinyl, piperidinyl, or (1-ethylpyrrolidin-2-yl)methanamine). In further embodiments, R⁶ is substituted with pyrrolidinyl or (1-ethylpyrrolidin-2-yl)methanamine). In yet further embodiments, R⁶ is substituted with alkylamino (e.g., —N(CH₂)₂N(C₄H₈)). In still further embodiments, R⁶ is substituted with alkylamido (e.g., N-diethylamido). In certain embodiments, R⁶ is substituted with nitro. In further embodiments, R⁶ is substituted with alkoxy. In yet further embodiments, R⁶ is substituted with amino (e.g., —NH₂). In still further embodiments, R⁶ is substituted with heterocyclyl.

In certain embodiments, the compound has a structure represented by formula lIb or a pharmaceutically acceptable salt thereof:

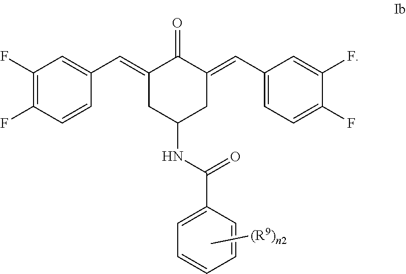

wherein
each R⁹ is independently selected from alkyl, alkenyl, alkynyl, halo, hydroxyl, carboxyl, acyl, acetyl, ester, thioester, alkoxy, phosphoryl, amino, amido, cyano, nitro, azido, alkylthio, cycloalkyl, alkylsulfonyl, sulfonamide, cycloalkyl, aryl, heteroaryl, and heterocyclyl; and
n is 1, 2, 3, 4, or 5.

In certain embodiments, the compound has a structure represented by Formula Ic, Formula IIa, or Formula IIIa, or a pharmaceutically acceptable salt thereof:

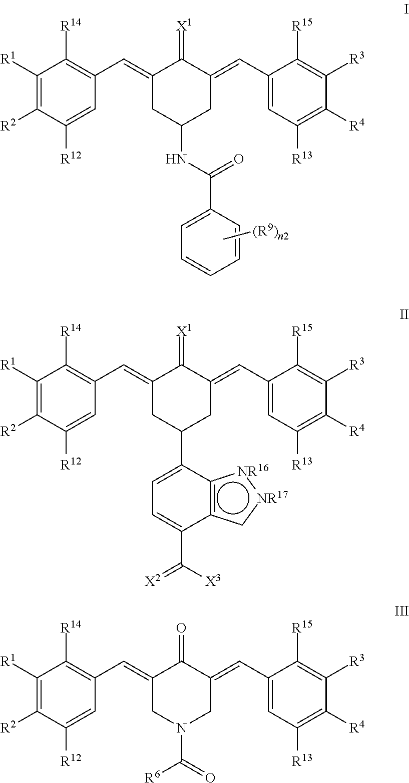

wherein
X³ is alkyl or N(R¹⁸)₂;
each R⁹ is independently selected from alkyl, alkenyl, alkynyl, halo, hydroxyl, carboxyl, acyl, acetyl, ester, thioester, alkoxy, phosphoryl, amino, amido, cyano, nitro, azido, alkylthio, cycloalkyl, alkylsulfonyl, sulfonamide, cycloalkyl, aryl, heteroaryl, and heterocyclyl;
each R¹⁶, R¹⁷, and R¹⁸ is independently H or alkyl; and
n2 is 1, 2, 3, 4, or 5.

In certain embodiments, the compound has a structure represented by Formula Ic, or a pharmaceutically acceptable salt thereof:

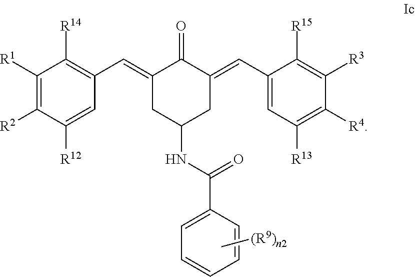

In certain embodiments, the compound has a structure represented by Formula IIa, or a pharmaceutically acceptable salt thereof:

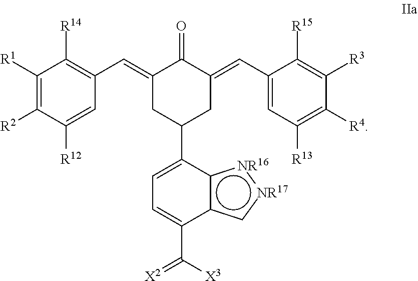

In certain embodiments, the compound has a structure represented by Formula IIIa, or a pharmaceutically acceptable salt thereof:

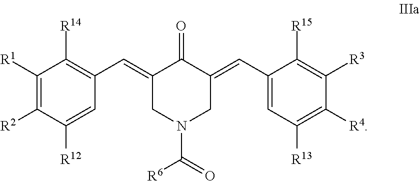

In certain embodiments, the compound has a structure represented by Formula Id, Formula IIb, Formula IIIb, or a pharmaceutically acceptable salt thereof:

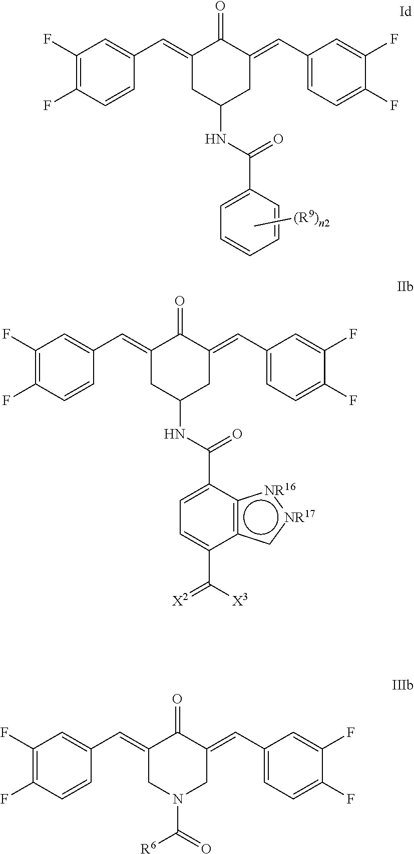

In certain embodiments, the compound has a structure represented by Formula Id, or a pharmaceutically acceptable salt thereof:

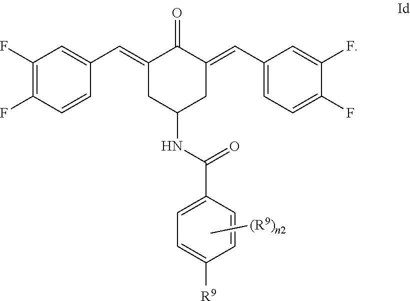

In certain embodiments, the compound has a structure represented by Formula IIb, or a pharmaceutically acceptable salt thereof:

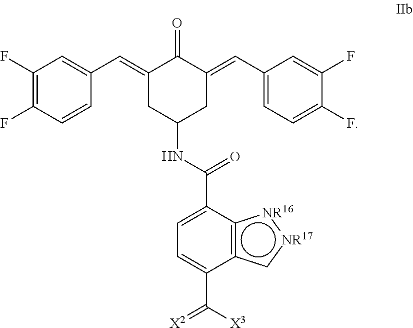

In certain embodiments, the compound has a structure represented by Formula IIIb, or a pharmaceutically acceptable salt thereof:

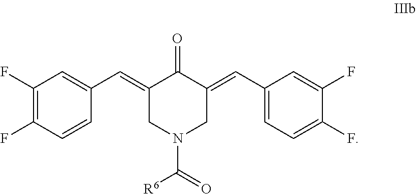

In certain embodiments, the compound has a structure represented by formula Ie, or a pharmaceutically acceptable salt thereof:

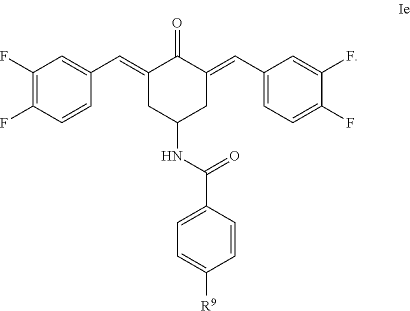

wherein
R⁹ is selected from alkyl, alkenyl, alkynyl, halo, hydroxyl, carboxyl, acyl, acetyl, ester, thioester, alkoxy, phosphoryl, amino, alkylamino, amido, cyano, nitro, azido, alkylthio, cycloalkyl, alkylsulfonyl, sulfonamide, carbamoylamino, cycloalkyl, aryl, heteroaryl, and heterocyclyl.

In certain embodiments, the compound has a structure represented by Formula If, Formula IIc, Formula IIIc, or a pharmaceutically acceptable salt thereof:

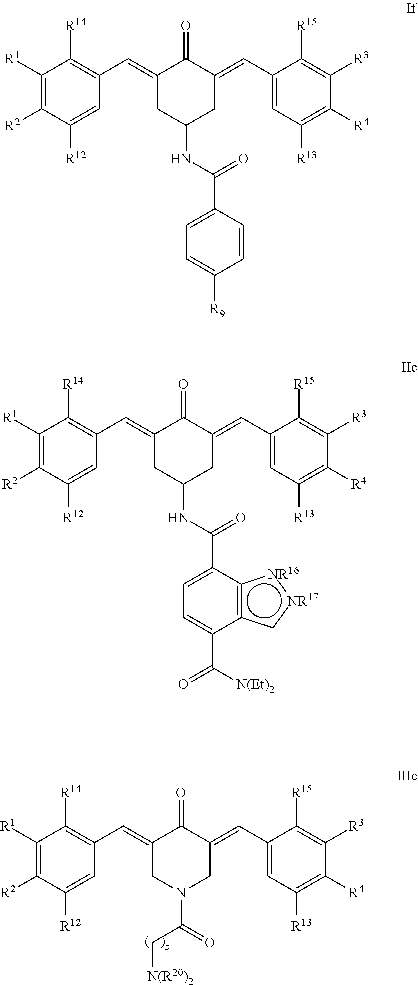

wherein
z is 0, 1, or 2;
R⁹ is selected from alkyl, alkenyl, alkynyl, halo, hydroxyl, carboxyl, acyl, acetyl, ester, thioester, alkoxy, phosphoryl, amino, alkylamino, amido, cyano, nitro, azido, alkylthio, cycloalkyl, alkylsulfonyl, sulfonamide, carbamoylamino, cycloalkyl, aryl, heteroaryl, and heterocyclyl; and R²⁰ is selected from H, cycloalkyl, cycloalkenyl, heterocycloalkyl, and heterocyclyl.

In certain embodiments, the compound has a structure represented by Formula If, or a pharmaceutically acceptable salt thereof:

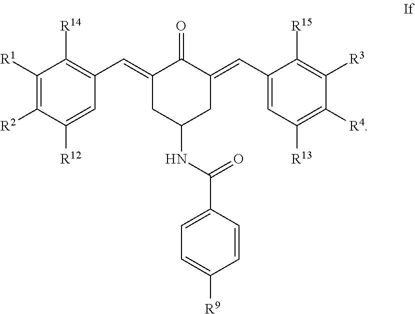

In certain embodiments, the compound has a structure represented by Formula IIc, or a pharmaceutically acceptable salt thereof:

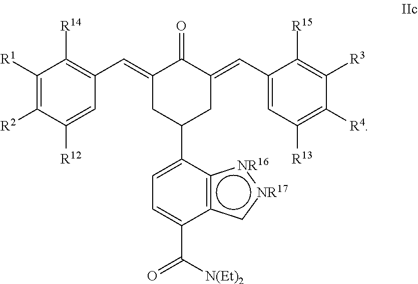

In certain embodiments, the compound has a structure represented by Formula IIIc, or a pharmaceutically acceptable salt thereof:

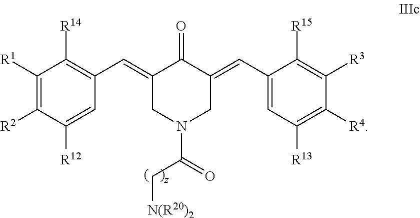

In certain embodiments, R¹ is fluoro. In certain embodiments, R² is fluoro. In certain embodiments, R³ is fluoro. In certain embodiments, R⁴ is fluoro. In certain embodiments, R¹² is fluoro. In certain embodiments, R¹³ is fluoro. In certain embodiments, R¹⁴ is fluoro. In certain embodiments, R¹⁵ is fluoro.

In certain embodiments, R⁹ is alkoxy. In further embodiments, R⁹ is amino. In yet further embodiments, R⁹ is aryl (e.g., phenyl) or heterocyclyl (e.g., pyrrolidinyl or as N-methyl piperazinyl). In still further embodiments, R⁹ is alkylamino.

In certain embodiments, R¹⁶ is H. In further embodiments, R¹⁶ is alkyl (e.g., methyl).

In certain embodiments, R¹⁷ is H. In further embodiments, R¹⁷ is alkyl (e.g., methyl).

In certain embodiments, z is 1.

In further embodiments, z is 2.

In certain embodiments, the compound has a structure represented by formula Ig or a pharmaceutically acceptable salt thereof:

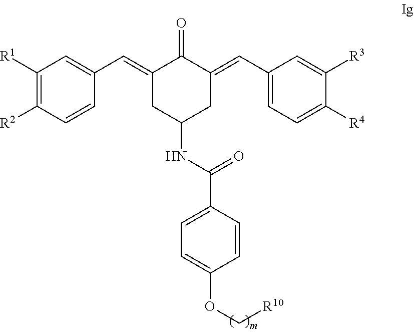

wherein
R¹⁰ is selected from alkyl, alkenyl, alkynyl, halo, hydroxyl, carboxyl, acyl, acetyl, ester, thioester, alkoxy, phosphoryl, amino, alkylamino, amido, carbamyl, cyano, nitro, azido, alkylthio, cycloalkyl, alkylsulfonyl, sulfonamide, carbamoylamino, cycloalkyl, aryl, heteroaryl, and heterocyclyl; and
m is 1, 2, 3, 4, 5, 6, 7, 8, 9, or 10.

In certain embodiments, the compound has a structure represented by Formula Ih, Formula IIId, or a pharmaceutically acceptable salt thereof:

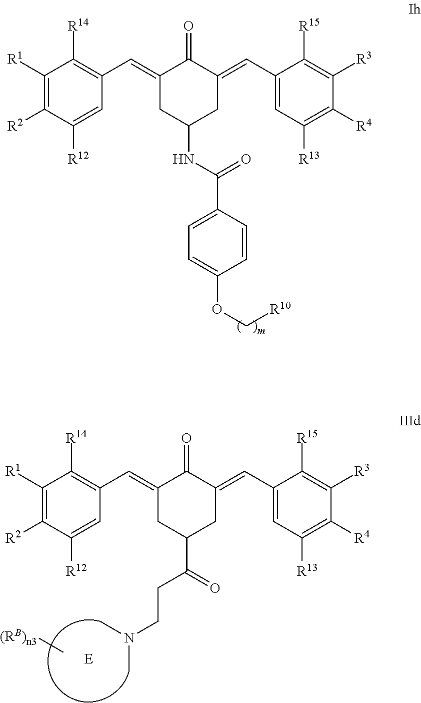

wherein
E is heterocyclyl;
R^{B} is selected from alkyl, amino, hydroxyl, halo, alkoxy, and sulfonyl;
R¹⁰ is selected from alkyl, alkenyl, alkynyl, halo, hydroxyl, carboxyl, acyl, acetyl, ester, thioester, alkoxy, phosphoryl, amino, alkylamino, amido, carbamyl, cyano, nitro, azido, alkylthio, cycloalkyl, alkylsulfonyl, sulfonamide, carbamoylamino, cycloalkyl, aryl, heteroaryl, and heterocyclyl;
n3 is 0, 1, 2, 3, 4, 5, 6, 7, 8, 9, or 10; and
m is 1, 2, 3, 4, 5, 6, 7, 8, 9, or 10.

In certain embodiments, the compound has a structure represented by Formula Ih, or a pharmaceutically acceptable salt thereof:

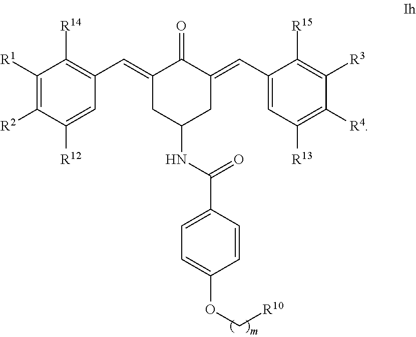

In certain embodiments, the compound has a structure represented by Formula IIId, or a pharmaceutically acceptable salt thereof:

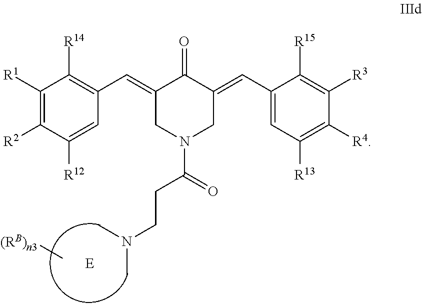

In certain embodiments, m is 1. In certain especially preferred embodiments, m is 2.

In certain embodiments, R¹ is chloro. In certain preferred embodiments, R¹ is —O(alkyl) (e.g., methoxy). In further preferred embodiments, R¹ is fluoro. In yet further preferred embodiments, R¹ is H.

In certain preferred embodiments, R² is fluoro.

In certain embodiments, R³ is chloro. In certain preferred embodiments, R³ is —O(alkyl) (e.g., methoxy). In further preferred embodiments, R³ is fluoro. In yet further embodiments, R³ is H. In certain preferred embodiments, R³ is H.

In certain preferred embodiments, R⁴ is fluoro.

In certain embodiments, R¹⁰ is selected from alkyl, alkenyl, alkynyl, halo, hydroxyl, carboxyl, acyl, acetyl, ester, thioester, alkoxy, phosphoryl, amino, alkylamino, amido, carbamyl, cyano, nitro, azido, alkylthio, cycloalkyl, alkylsulfonyl, sulfonamide, carbamoylamino, cycloalkyl, aryl, heteroaryl, and heterocyclyl. In certain preferred embodiments, R¹⁰ is alkylamino (e.g., ethylamino, diethylamino, or —N(CH₂)₂O(CH₃)). In certain preferred embodiments, R¹⁰ is ethylamino. In yet further preferred embodiments, R¹⁰ is diethylamino. In certain preferred embodiments, R¹⁰ is heterocyclyl (e.g., 2-oxa-6-azaspiro[3.3]heptyl, piperidinyl, pyrrolidinyl, octahydrocyclopenta[c]pyrrolidinyl, or 3-azabicyclo[3.1.0]hexyl). In certain preferred embodiments, R¹⁰ is piperidinyl. In certain embodiments, R¹⁰ is 2-oxa-6-azaspiro[3.3]heptyl. In certain preferred embodiments, R¹⁰ is pyrrolidinyl. In certain embodiments, R¹⁰ is 3-azabicyclo[3.1.0]hexyl. In further embodiments, R¹⁰ is octahydrocyclopenta[c]pyrrolidinyl.

In certain preferred embodiments, R¹² is fluoro. In certain preferred embodiments, R¹³ is fluoro. In certain preferred embodiments, R¹⁴ is fluoro. In certain preferred embodiments, R¹⁵ is fluoro.

In certain embodiments, E is heterocyclyl (e.g., pyrrolidinyl, azetidinyl, octahydrocyclopenta[c]pyrrolidinyl, octahydropyrrolo[1,2-a]pyrazinyl, or 3-azabicyclo[3.1.0]hexyl).

In certain embodiments, R^{B} is alkyl (e.g., methyl). In further embodiments, R^{B} is halo (e.g., fluoro). In yet further embodiments, R^{B} is amino (e.g., dimethylamino). In still further embodiments, R^{B} is alkoxy (e.g., methoxy). In certain embodiments, R^{B} is sulfonyl (e.g., —S(O)₂CH₃)).

In certain embodiments, n3 is 0. In further embodiments, n3 is 1.

In certain embodiments, the compound has a structure represented by formula II or a pharmaceutically acceptable salt thereof:

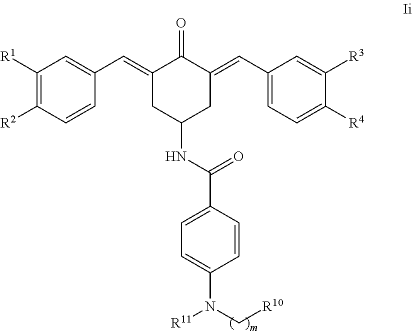

wherein
R¹⁰ is selected from alkyl, alkenyl, alkynyl, halo, hydroxyl, carboxyl, acyl, acetyl, ester, thioester, alkoxy, phosphoryl, amino, alkylamino, amido, carbamyl, cyano, nitro, azido, alkylthio, cycloalkyl, alkylsulfonyl, sulfonamide, carbamoylamino, cycloalkyl, aryl, heteroaryl, and heterocyclyl;
R¹¹ is H, alkyl, or aralkyl; and
m is 1, 2, 3, 4, 5, 6, 7, 8, 9, or 10.

In certain embodiments, the compound has a structure represented by formula Ij, or a pharmaceutically acceptable salt thereof:

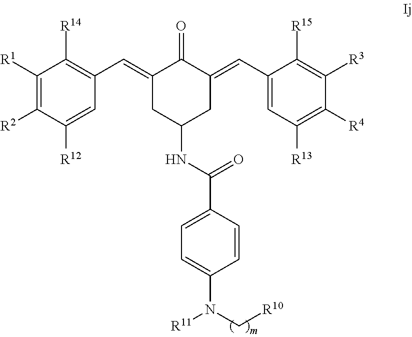

wherein
R¹⁰ is selected from alkyl, alkenyl, alkynyl, halo, hydroxyl, carboxyl, acyl, acetyl, ester, thioester, alkoxy, phosphoryl, amino, alkylamino, amido, carbamyl, cyano, nitro, azido, alkylthio, cycloalkyl, alkylsulfonyl, sulfonamide, carbamoylamino, cycloalkyl, aryl, heteroaryl, and heterocyclyl;
R¹¹ is H, alkyl, or aralkyl; and
m is 1, 2, 3, 4, 5, 6, 7, 8, 9, or 10.

In certain embodiments, m is 1. In further embodiments, m is 2.

In yet further embodiments, m is 3.

In certain embodiments, R¹⁰ is amino. In further embodiments, R¹⁰ is NH₂, alkylamino (e.g., propylamino, pentylamino, or hexylamino), alkyloxyalkylamino (e.g., methoxypropylamino, methoxyethylamino, methoxyethylamino, or ethoxyethylamino), dialkylamino (e.g., diethylamino, dipropylamino, diisopropylamino, dibutylamino, dipentylamino, dihexylamino, or dioctylamino), dialkenylamino (e.g., diallylamino), dialkynylamino (e.g., dibutynylamino), dialkyloxyalkylamino (e.g., dimethoxyethylamino), heterocyclylalkylamino (e.g., tetrahydrofuranylmethanamino, N-methylpyrrolidinylmethanamino, or N-ethylpyrrolidinylmethanamino), dialkoxyalkylamino (e.g., diethoxyethylamino), cycloalkylamino (e.g., dicyclohexylamino), aralkylamino (e.g., dibenzylamino), (alkyl)(cycloalkyl)amino (e.g., (methyl)(cyclohexyl)amino or (ethyl)(cyclohexyl)amino). In yet further embodiments, R¹⁰ is heterocyclyl (e.g., pyrrolidinyl, piperidinyl, piperazinyl, such as N-methyl piperazinyl, azepanyl, azocanyl, morpholinyl, oxazolidinonyl, and phthalimidyl). In still further embodiments, R¹⁰ is heterocyclyl (e.g., pyrrolidinyl, such as N-methyl pyrrolidinyl, piperidinyl, azetidinyl, octahydrocyclopenta[c]pyrrolidinyl, octahydropyrrolo[1,2-a]pyrazinyl, 3-azabicyclo[3.1.0]hexyl, 2-oxa-6-azaspiro[3.3]heptyl, hexahydro-5H-[1,4]dioxino[2,3-c]-pyrrolyl, piperazinyl, such as N-methyl piperazinyl, azepanyl, azocanyl, morpholinyl, oxazolidinonyl, and phthalimidyl). In certain embodiments, R¹⁰ is carbamyl (e.g., tert-butyl carbamoyl). In further embodiments, R¹⁰ is heteroaryl (e.g., triazolyl).

In certain especially preferred embodiments, R¹⁰ is N-ethylpyrrolidinylmethanamino.

In certain embodiments, the compound has a structure represented by formula Ik or a pharmaceutically acceptable salt thereof:

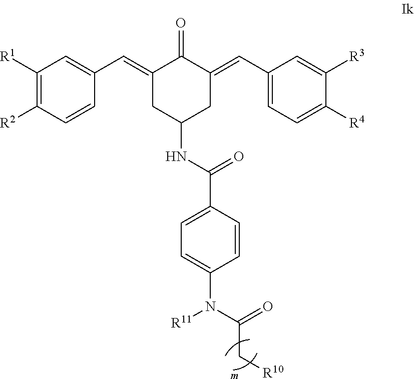

wherein
R¹⁰ is selected from alkyl, alkenyl, alkynyl, halo, hydroxyl, carboxyl, acyl, acetyl, ester, thioester, alkoxy, phosphoryl, amino, alkylamino, amido, carbamyl, cyano, nitro, azido, alkylthio, cycloalkyl, alkylsulfonyl, sulfonamide, carbamoylamino, cycloalkyl, aryl, heteroaryl, and heterocyclyl;
R¹¹ is H, alkyl, or aralkyl; and
m is 0, 1, 2, 3, 4, 5, 6, 7, 8, 9, or 10.

In certain embodiments, the compound has a structure represented by formula II or a

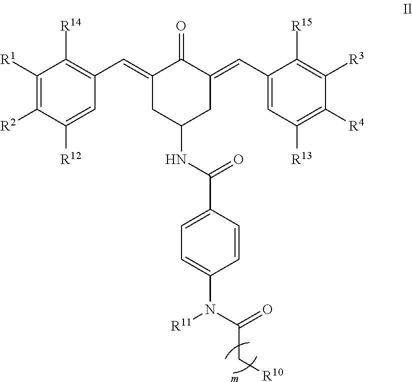

wherein
R¹⁰ is selected from alkyl, alkenyl, alkynyl, halo, hydroxyl, carboxyl, acyl, acetyl, ester, thioester, alkoxy, phosphoryl, amino, alkylamino, amido, carbamyl, cyano, nitro, azido, alkylthio, cycloalkyl, alkylsulfonyl, sulfonamide, carbamoylamino, cycloalkyl, aryl, heteroaryl, and heterocyclyl;
R¹¹ is H, alkyl, or aralkyl; and
m is 0, 1, 2, 3, 4, 5, 6, 7, 8, 9, or 10.

In certain embodiments, m is 0. In further embodiments, m is 1.

In certain embodiments, R¹⁰ is heterocyclyl (e.g., azetidine, pyrrolidinyl, pyrrolidinonyl, morpholinyl, piperidinyl, piperazinyl (such as N-methyl piperazinyl), or isoindolinyl). In certain embodiments, R¹⁰ is amino. In further embodiments, R¹⁰ is alkylamino (e.g., diethylamino or dibutylamino), or alkoxyalkylamino (e.g., dimethoxyethylamino). In certain embodiments, R¹⁰ is heteroaryl (e.g., isoindoline). In further embodiments, R¹⁰ is substituted with alkyl, alkenyl, alkynyl, aralkyl, halo, hydroxyl, carboxyl, acyl, acetyl, ester, thioester, alkoxy, phosphoryl, amino, alkylamino, amido, carbamyl, cyano, nitro, azido, alkylthio, cycloalkyl, alkylsulfonyl, sulfonamide, carbamoylamino, cycloalkyl, aryl, heteroaryl, and heterocyclyl. In yet further embodiments, R¹⁰ is substituted with alkyl (e.g., trifluoromethyl or thiophenylethyl). In still further embodiments, R¹⁰ is substituted with aryl (e.g., phenyl). In certain embodiments, R¹⁰ is substituted with acyl (e.g., cyclopropylmethanonyl). In certain embodiments, R¹⁰ is substituted with amino (e.g., dimethylamino). In certain embodiments, R¹⁰ is substituted with heterocyclyl (e.g., benzopyranyl or pyrrolidinyl). In certain embodiments, R¹⁰ is substituted with amido (e.g., pyridinylmethylamido). In certain embodiments, R¹⁰ is substituted with ester (e.g., tert-butyl ester). In certain embodiments, R¹⁰ is substituted with aralkyl.

In certain embodiments, R¹¹ is H. In certain embodiments, R¹¹ is alkyl (e.g., ethyl).

In certain embodiments, the compound is selected from

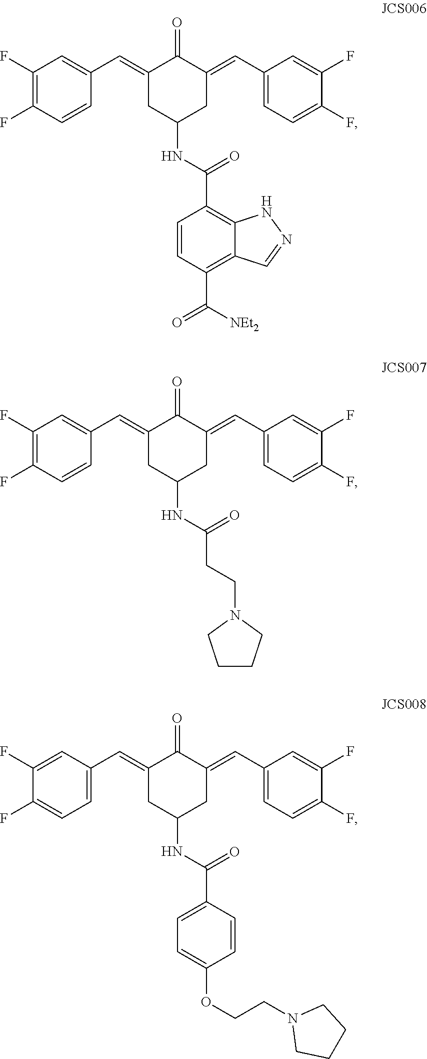

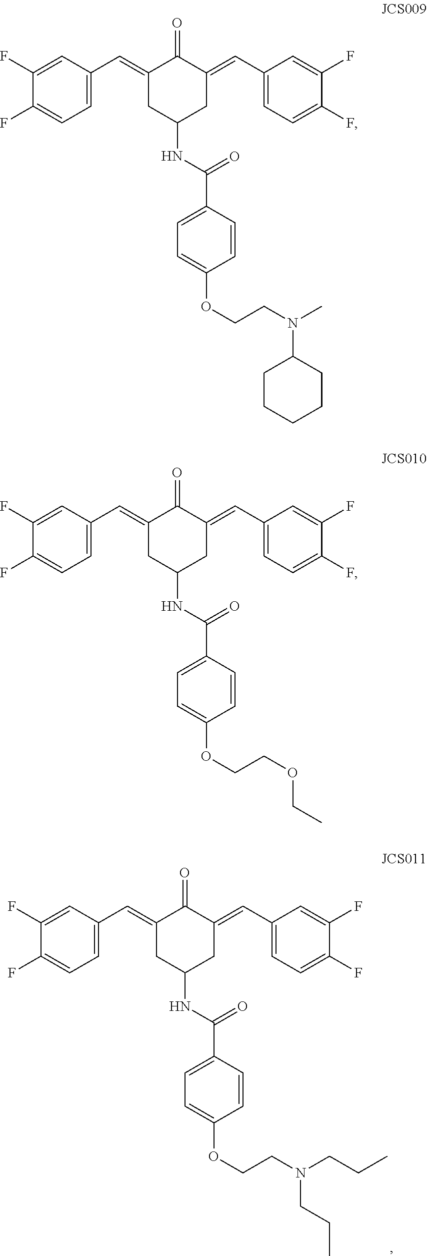 [SMILES: CN(CCOc1ccc(C(=O)NC2C/C(=C\c3ccc(F)c(F)c3)C(=O)/C(=C/c3ccc(F)c(F)c3)C2)cc1)C1CCCCC1 ; CCOCCOc1ccc(C(=O)NC2C/C(=C\c3ccc(F)c(F)c3)C(=O)/C(=C/c3ccc(F)c(F)c3)C2)cc1 ; CCCN(CCC)CCOc1ccc(C(=O)NC2C/C(=C\c3ccc(F)c(F)c3)C(=O)/C(=C/c3ccc(F)c(F)c3)C2)cc1]

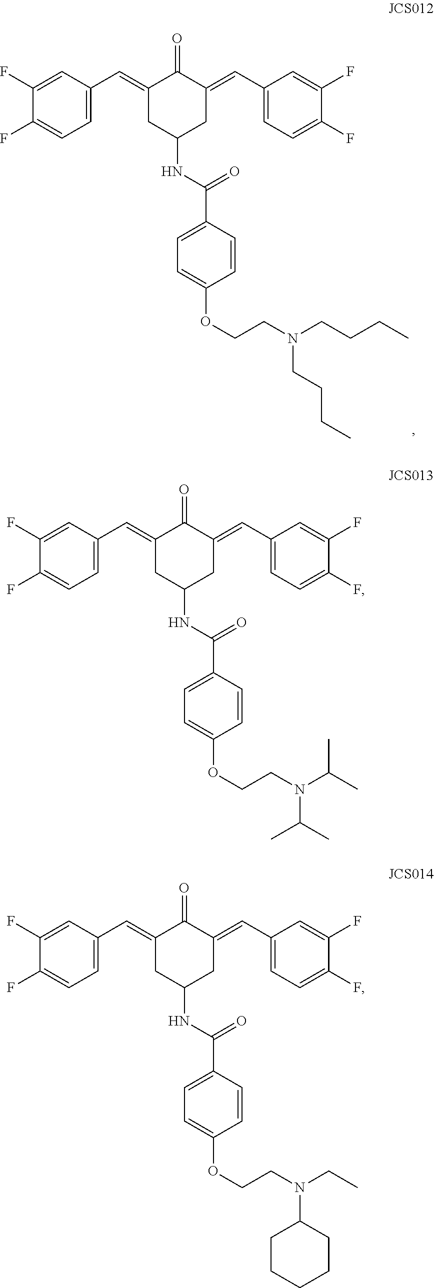 [SMILES: CCCCN(CCCC)CCOc1ccc(C(=O)NC2C/C(=C\c3ccc(F)c(F)c3)C(=O)/C(=C/c3ccc(F)c(F)c3)C2)cc1 ; CC(C)N(CCOc1ccc(C(=O)NC2C/C(=C\c3ccc(F)c(F)c3)C(=O)/C(=C/c3ccc(F)c(F)c3)C2)cc1)C(C)C ; CCN(CCOc1ccc(C(=O)NC2C/C(=C\c3ccc(F)c(F)c3)C(=O)/C(=C/c3ccc(F)c(F)c3)C2)cc1)C1CCCCC1]

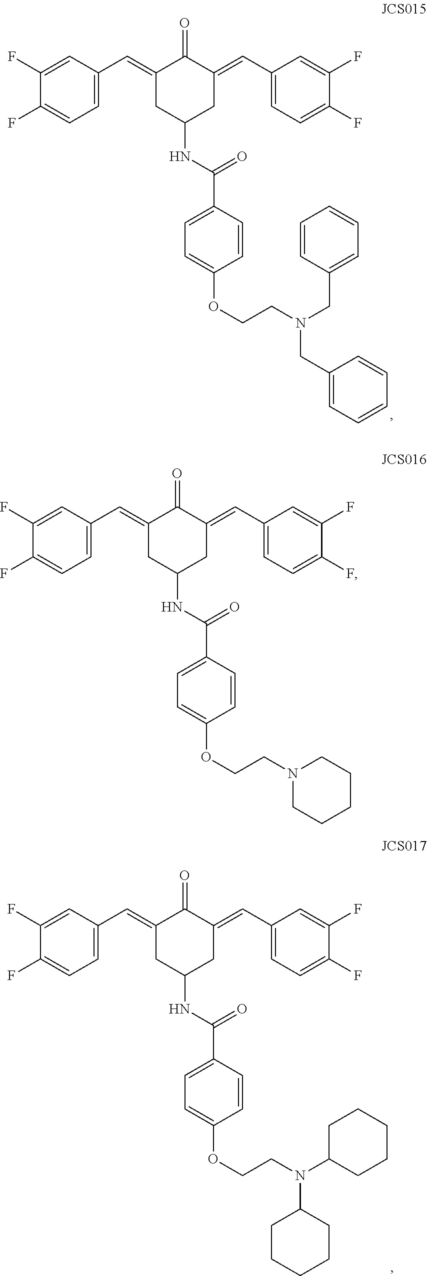 [SMILES: O=C1/C(=C/c2ccc(F)c(F)c2)CC(NC(=O)c2ccc(OCCN(Cc3ccccc3)Cc3ccccc3)cc2)C/C1=C\c1ccc(F)c(F)c1 ; O=C1/C(=C/c2ccc(F)c(F)c2)CC(NC(=O)c2ccc(OCCN3CCCCC3)cc2)C/C1=C\c1ccc(F)c(F)c1 ; O=C1/C(=C/c2ccc(F)c(F)c2)CC(NC(=O)c2ccc(OCCN(C3CCCCC3)C3CCCCC3)cc2)C/C1=C\c1ccc(F)c(F)c1]

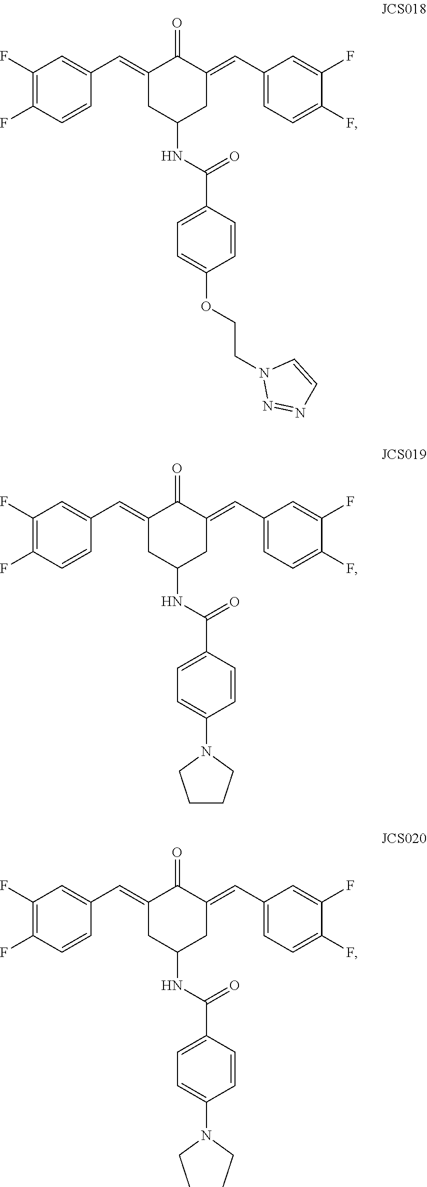 [SMILES: O=C1/C(=C/c2ccc(F)c(F)c2)CC(NC(=O)c2ccc(OCCn3ccnn3)cc2)C/C1=C\c1ccc(F)c(F)c1 ; O=C1/C(=C/c2ccc(F)c(F)c2)CC(NC(=O)c2ccc(N3CCCC3)cc2)C/C1=C\c1ccc(F)c(F)c1 ; CCN(CC)c1ccc(C(=O)NC2C/C(=C\c3ccc(F)c(F)c3)C(=O)/C(=C/c3ccc(F)c(F)c3)C2)cc1]

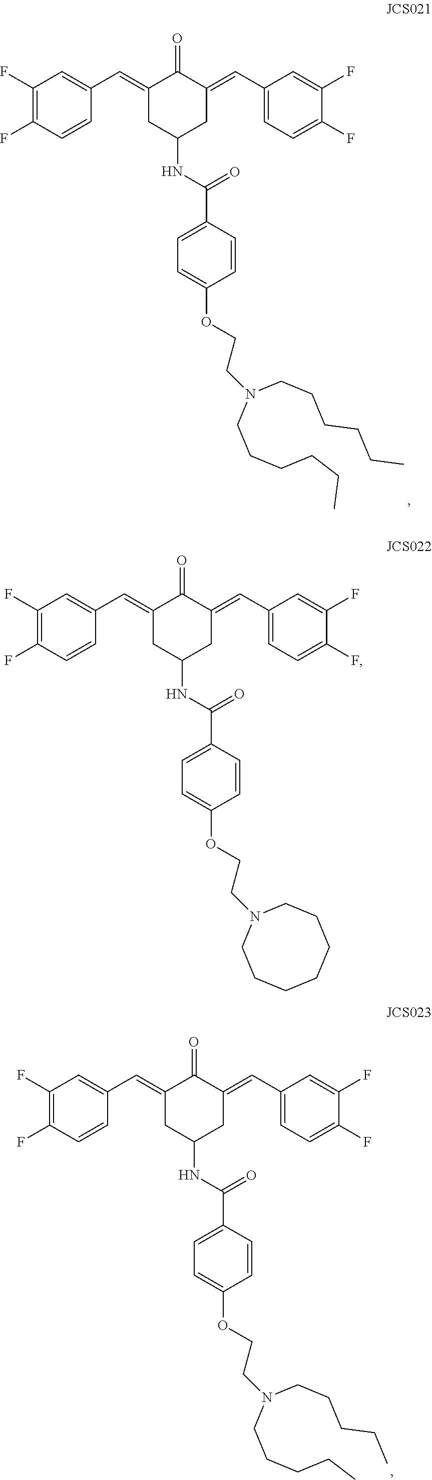 [SMILES: CCCCCCN(CCCCCC)CCOc1ccc(C(=O)NC2C/C(=C\c3ccc(F)c(F)c3)C(=O)/C(=C/c3ccc(F)c(F)c3)C2)cc1 ; O=C1/C(=C/c2ccc(F)c(F)c2)CC(NC(=O)c2ccc(OCCN3CCCCCCC3)cc2)C/C1=C\c1ccc(F)c(F)c1 ; CCCCCN(CCCCC)CCOc1ccc(C(=O)NC2C/C(=C\c3ccc(F)c(F)c3)C(=O)/C(=C/c3ccc(F)c(F)c3)C2)cc1]

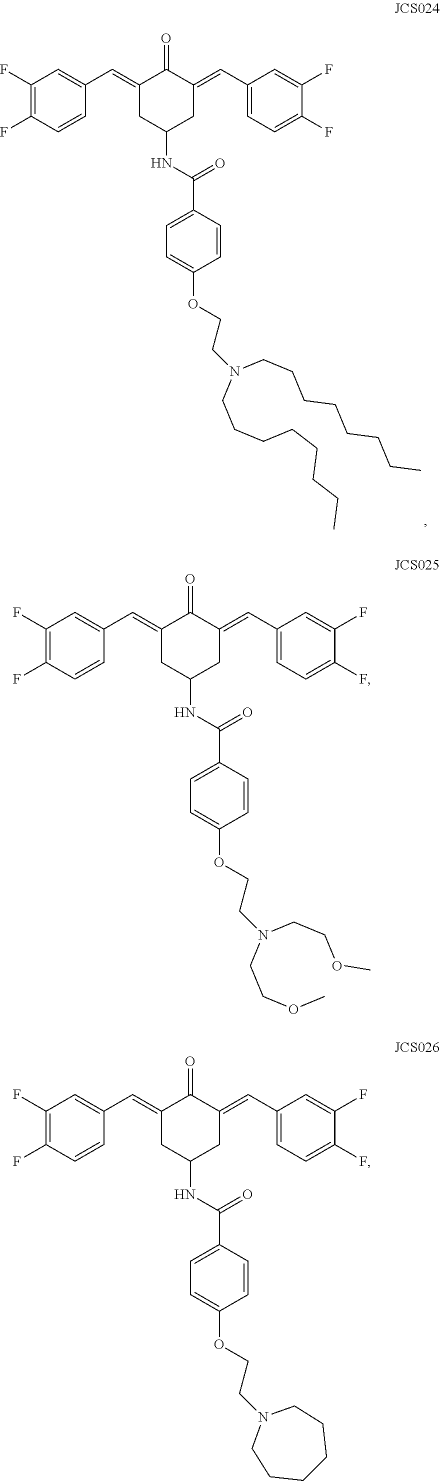 [SMILES: CCCCCCCCN(CCCCCCCC)CCOc1ccc(C(=O)NC2C/C(=C\c3ccc(F)c(F)c3)C(=O)/C(=C/c3ccc(F)c(F)c3)C2)cc1 ; COCCN(CCOC)CCOc1ccc(C(=O)NC2C/C(=C\c3ccc(F)c(F)c3)C(=O)/C(=C/c3ccc(F)c(F)c3)C2)cc1 ; O=C1/C(=C/c2ccc(F)c(F)c2)CC(NC(=O)c2ccc(OCCN3CCCCCC3)cc2)C/C1=C\c1ccc(F)c(F)c1]

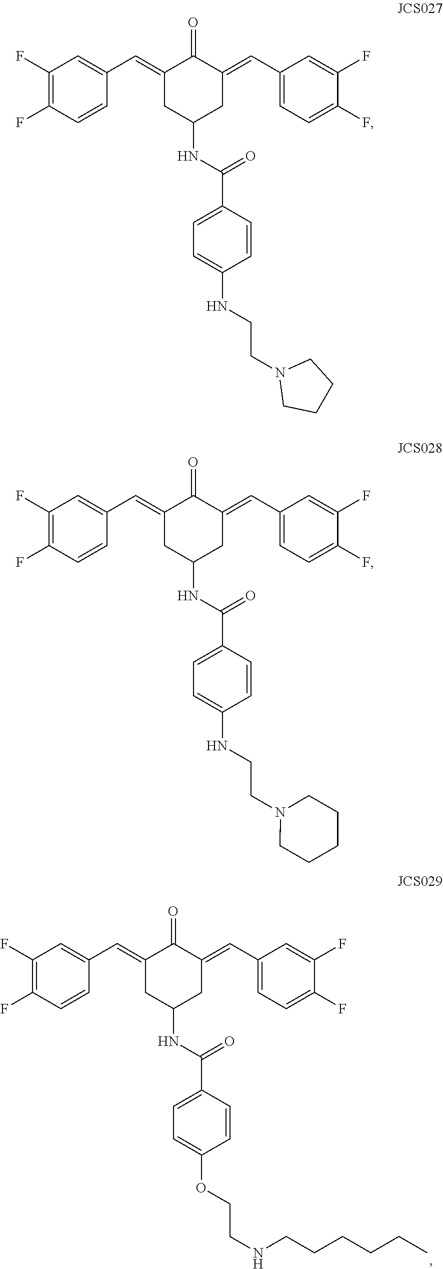 [SMILES: O=C1/C(=C/c2ccc(F)c(F)c2)CC(NC(=O)c2ccc(NCCN3CCCC3)cc2)C/C1=C\c1ccc(F)c(F)c1 ; O=C1/C(=C/c2ccc(F)c(F)c2)CC(NC(=O)c2ccc(NCCN3CCCCC3)cc2)C/C1=C\c1ccc(F)c(F)c1 ; CCCCCCNCCOc1ccc(C(=O)NC2C/C(=C\c3ccc(F)c(F)c3)C(=O)/C(=C/c3ccc(F)c(F)c3)C2)cc1]

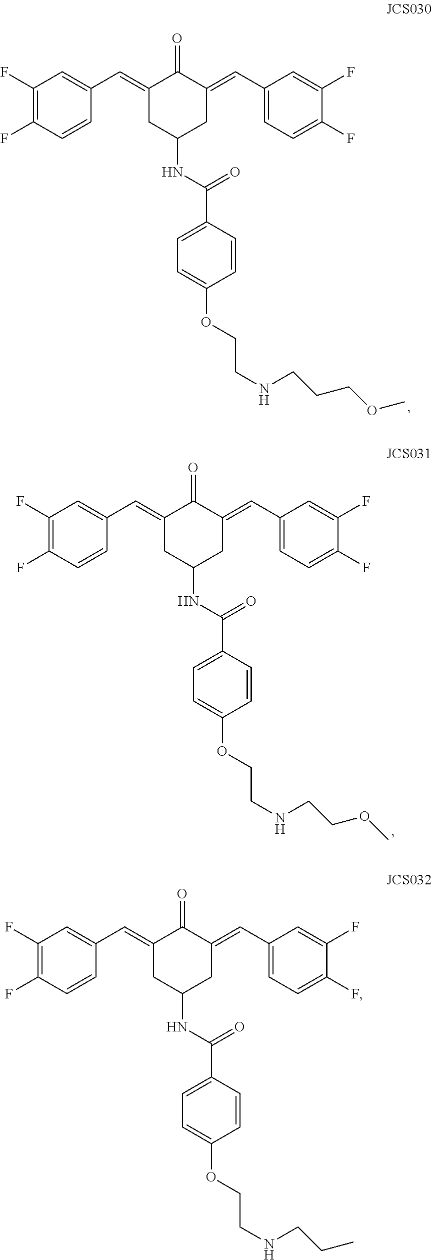 [SMILES: COCCCNCCOc1ccc(C(=O)NC2C/C(=C\c3ccc(F)c(F)c3)C(=O)/C(=C/c3ccc(F)c(F)c3)C2)cc1 ; COCCNCCOc1ccc(C(=O)NC2C/C(=C\c3ccc(F)c(F)c3)C(=O)/C(=C/c3ccc(F)c(F)c3)C2)cc1 ; CCCNCCOc1ccc(C(=O)NC2C/C(=C\c3ccc(F)c(F)c3)C(=O)/C(=C/c3ccc(F)c(F)c3)C2)cc1]

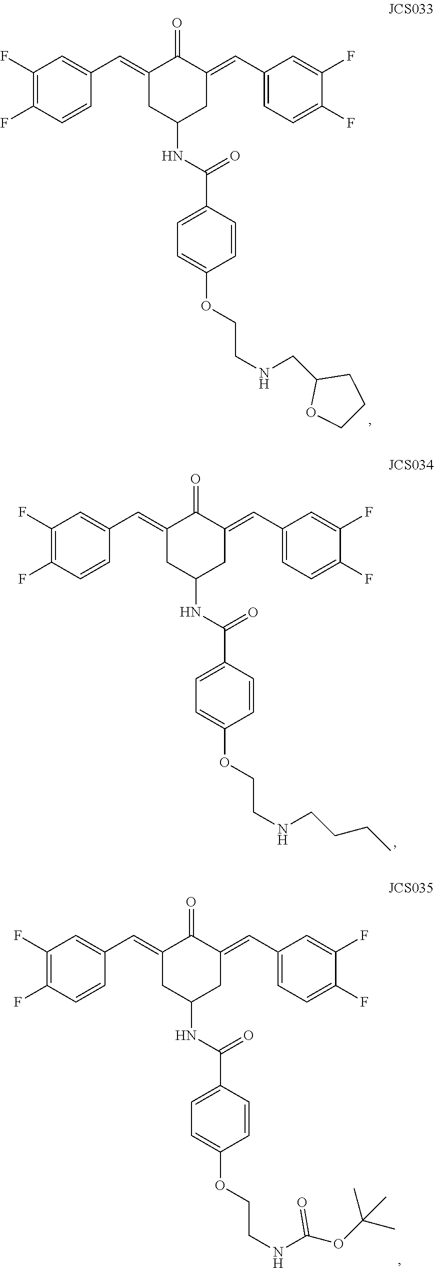 [SMILES: O=C1/C(=C/c2ccc(F)c(F)c2)CC(NC(=O)c2ccc(OCCNCC3CCCO3)cc2)C/C1=C\c1ccc(F)c(F)c1 ; CCCCNCCOc1ccc(C(=O)NC2C/C(=C\c3ccc(F)c(F)c3)C(=O)/C(=C/c3ccc(F)c(F)c3)C2)cc1 ; CC(C)(C)OC(=O)NCCOc1ccc(C(=O)NC2C/C(=C\c3ccc(F)c(F)c3)C(=O)/C(=C/c3ccc(F)c(F)c3)C2)cc1]

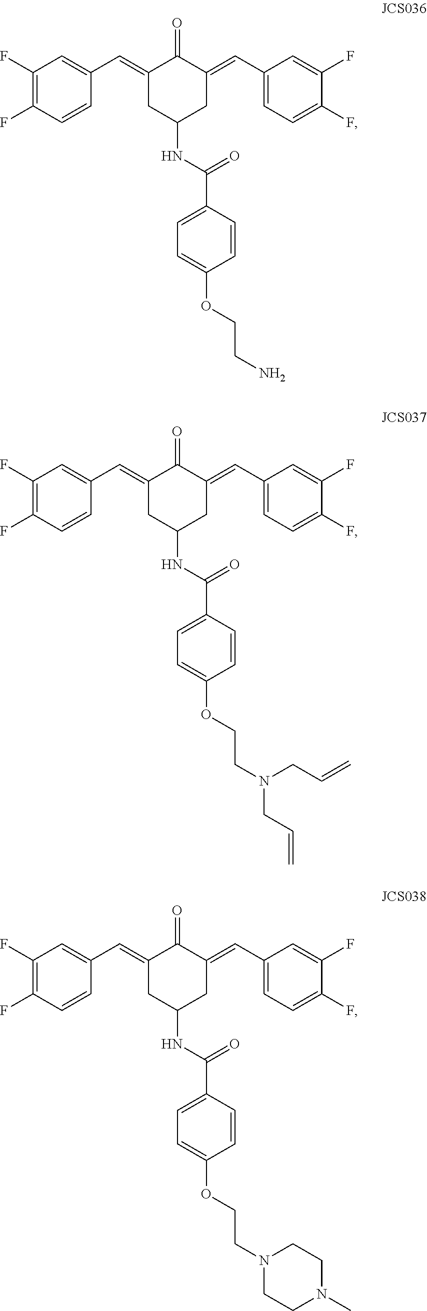 [SMILES: NCCOc1ccc(C(=O)NC2C/C(=C\c3ccc(F)c(F)c3)C(=O)/C(=C/c3ccc(F)c(F)c3)C2)cc1 ; C=CCN(CC=C)CCOc1ccc(C(=O)NC2C/C(=C\c3ccc(F)c(F)c3)C(=O)/C(=C/c3ccc(F)c(F)c3)C2)cc1 ; CN1CCN(CCOc2ccc(C(=O)NC3C/C(=C\c4ccc(F)c(F)c4)C(=O)/C(=C/c4ccc(F)c(F)c4)C3)cc2)CC1]

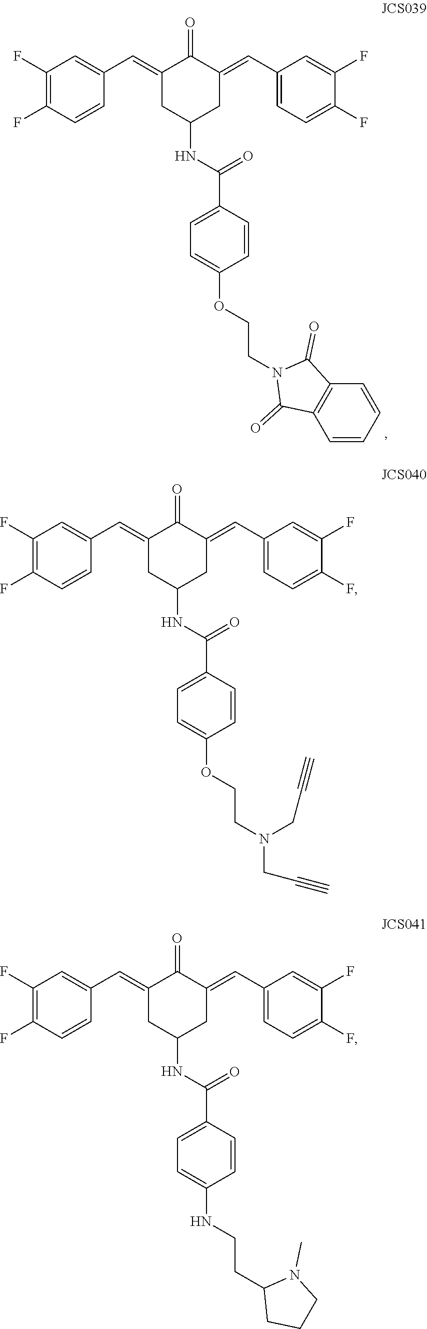 [SMILES: O=C1/C(=C/c2ccc(F)c(F)c2)CC(NC(=O)c2ccc(OCCN3C(=O)c4ccccc4C3=O)cc2)C/C1=C\c1ccc(F)c(F)c1 ; C#CCN(CC#C)CCOc1ccc(C(=O)NC2C/C(=C\c3ccc(F)c(F)c3)C(=O)/C(=C/c3ccc(F)c(F)c3)C2)cc1 ; CN1CCCC1CCNc1ccc(C(=O)NC2C/C(=C\c3ccc(F)c(F)c3)C(=O)/C(=C/c3ccc(F)c(F)c3)C2)cc1]

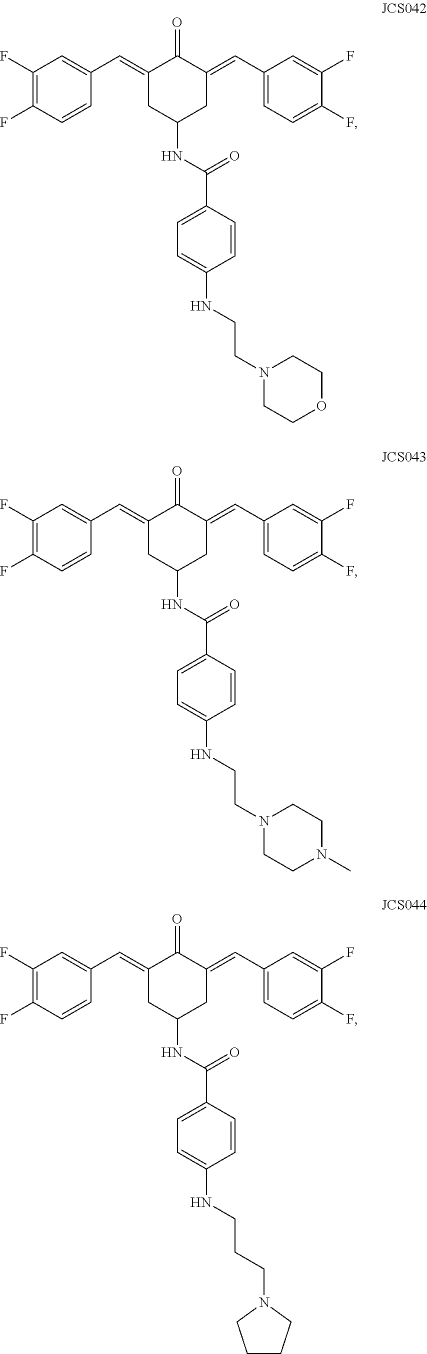 [SMILES: O=C1/C(=C/c2ccc(F)c(F)c2)CC(NC(=O)c2ccc(NCCN3CCOCC3)cc2)C/C1=C\c1ccc(F)c(F)c1 ; CN1CCN(CCNc2ccc(C(=O)NC3C/C(=C\c4ccc(F)c(F)c4)C(=O)/C(=C/c4ccc(F)c(F)c4)C3)cc2)CC1 ; O=C1/C(=C/c2ccc(F)c(F)c2)CC(NC(=O)c2ccc(NCCCN3CCCC3)cc2)C/C1=C\c1ccc(F)c(F)c1]

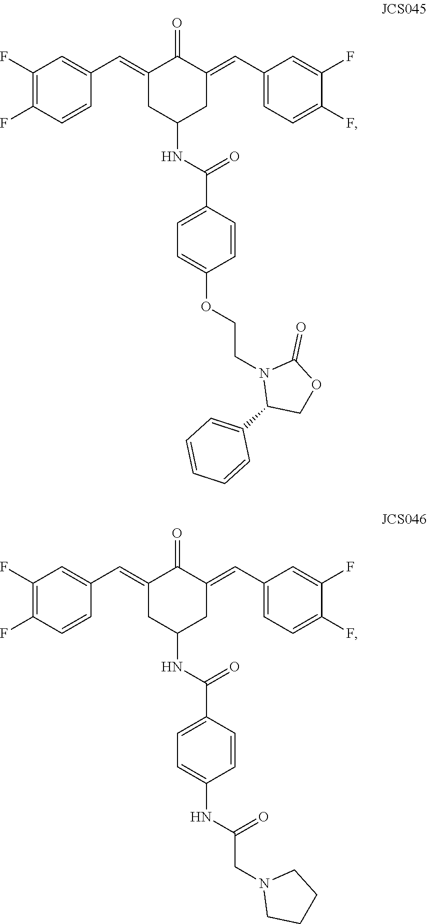 [SMILES: O=C1/C(=C/c2ccc(F)c(F)c2)CC(NC(=O)c2ccc(OCCN3C(=O)OC[C@@H]3c3ccccc3)cc2)C/C1=C\c1ccc(F)c(F)c1 ; O=C(CN1CCCC1)Nc1ccc(C(=O)NC2C/C(=C\c3ccc(F)c(F)c3)C(=O)/C(=C/c3ccc(F)c(F)c3)C2)cc1]

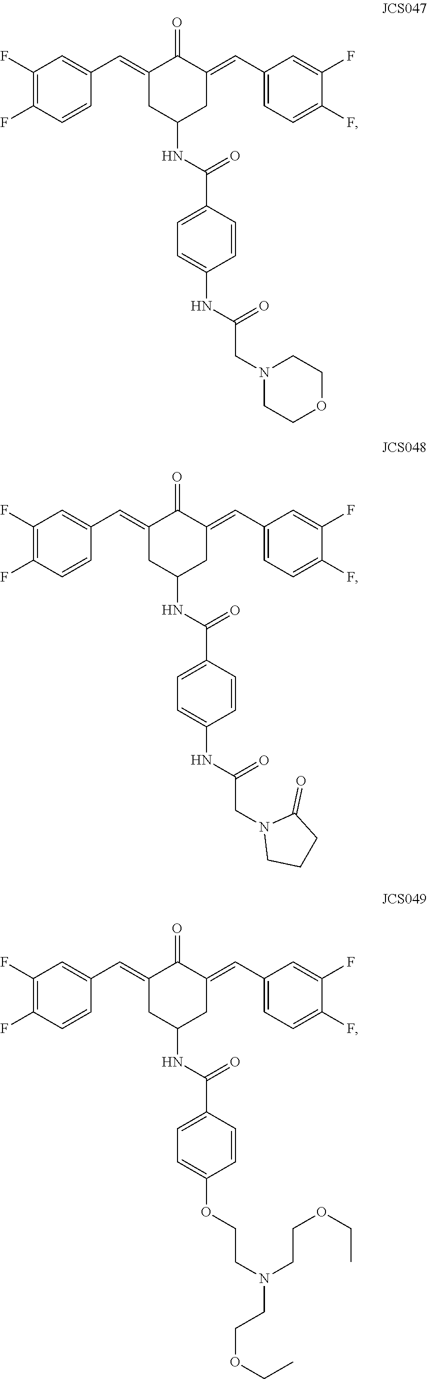 [SMILES: O=C(CN1CCOCC1)Nc1ccc(C(=O)NC2C/C(=C\c3ccc(F)c(F)c3)C(=O)/C(=C/c3ccc(F)c(F)c3)C2)cc1 ; O=C(CN1CCCC1=O)Nc1ccc(C(=O)NC2C/C(=C\c3ccc(F)c(F)c3)C(=O)/C(=C/c3ccc(F)c(F)c3)C2)cc1 ; CCOCCN(CCOCC)CCOc1ccc(C(=O)NC2C/C(=C\c3ccc(F)c(F)c3)C(=O)/C(=C/c3ccc(F)c(F)c3)C2)cc1]

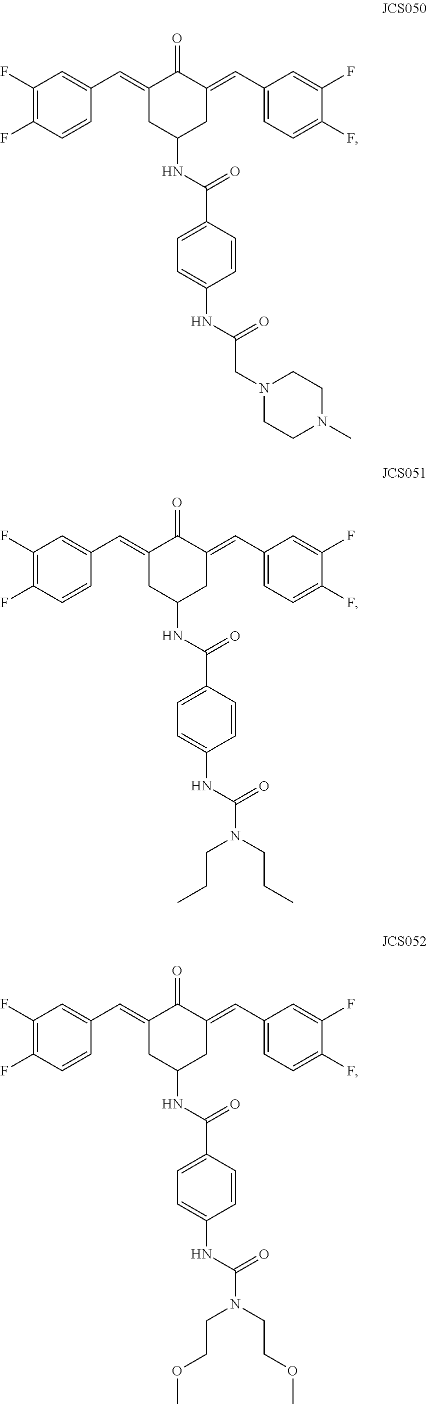 [SMILES: CN1CCN(CC(=O)Nc2ccc(C(=O)NC3C/C(=C\c4ccc(F)c(F)c4)C(=O)/C(=C/c4ccc(F)c(F)c4)C3)cc2)CC1 ; CCCN(CCC)C(=O)Nc1ccc(C(=O)NC2C/C(=C\c3ccc(F)c(F)c3)C(=O)/C(=C/c3ccc(F)c(F)c3)C2)cc1 ; COCCN(CCOC)C(=O)Nc1ccc(C(=O)NC2C/C(=C\c3ccc(F)c(F)c3)C(=O)/C(=C/c3ccc(F)c(F)c3)C2)cc1]

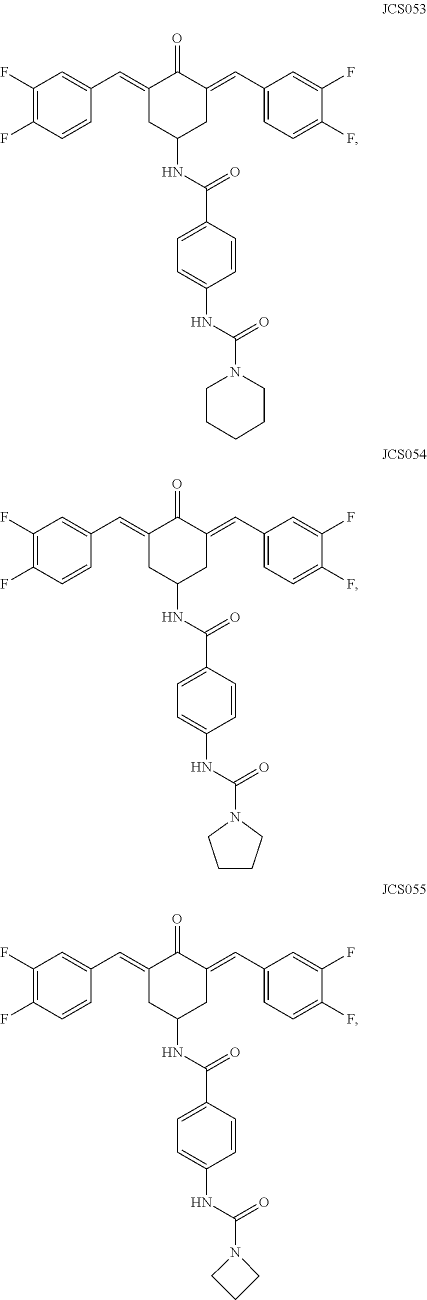 [SMILES: O=C1/C(=C/c2ccc(F)c(F)c2)CC(NC(=O)c2ccc(NC(=O)N3CCCCC3)cc2)C/C1=C\c1ccc(F)c(F)c1 ; O=C1/C(=C/c2ccc(F)c(F)c2)CC(NC(=O)c2ccc(NC(=O)N3CCCC3)cc2)C/C1=C\c1ccc(F)c(F)c1 ; O=C1/C(=C/c2ccc(F)c(F)c2)CC(NC(=O)c2ccc(NC(=O)N3CCC3)cc2)C/C1=C\c1ccc(F)c(F)c1]

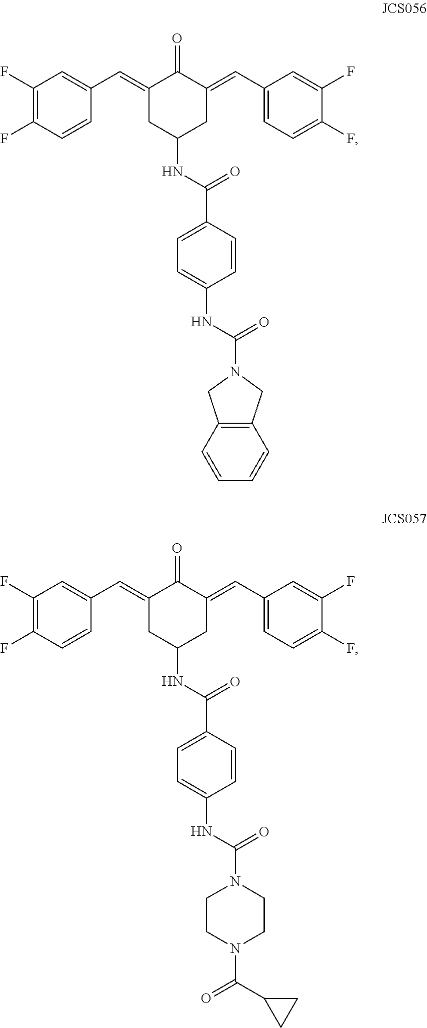 [SMILES: O=C1/C(=C/c2ccc(F)c(F)c2)CC(NC(=O)c2ccc(NC(=O)N3Cc4ccccc4C3)cc2)C/C1=C\c1ccc(F)c(F)c1 ; O=C1/C(=C/c2ccc(F)c(F)c2)CC(NC(=O)c2ccc(NC(=O)N3CCN(C(=O)C4CC4)CC3)cc2)C/C1=C\c1ccc(F)c(F)c1]

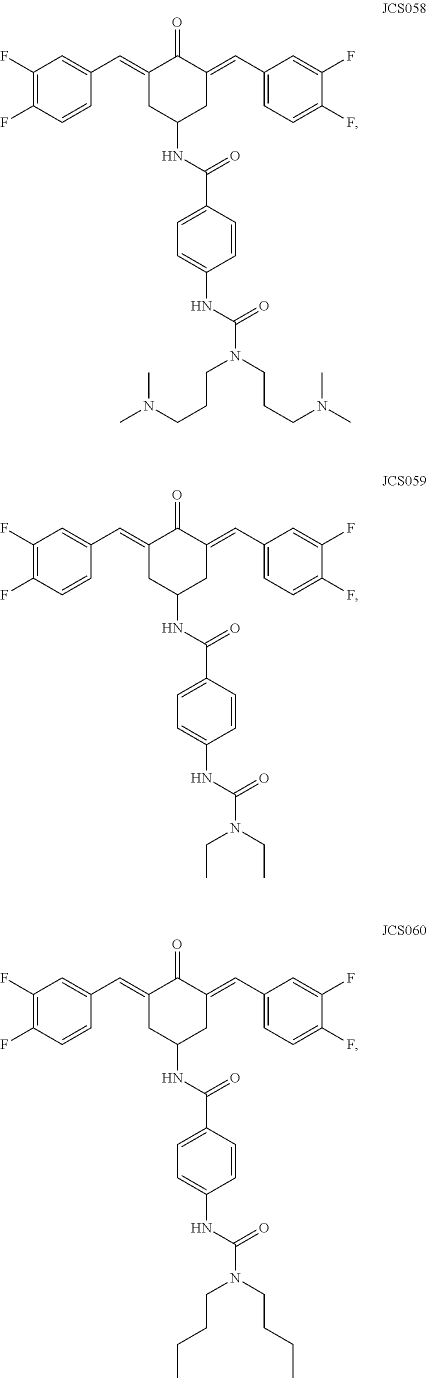 [SMILES: CN(C)CCCN(CCCN(C)C)C(=O)Nc1ccc(C(=O)NC2C/C(=C\c3ccc(F)c(F)c3)C(=O)/C(=C/c3ccc(F)c(F)c3)C2)cc1 ; CCN(CC)C(=O)Nc1ccc(C(=O)NC2C/C(=C\c3ccc(F)c(F)c3)C(=O)/C(=C/c3ccc(F)c(F)c3)C2)cc1 ; CCCCN(CCCC)C(=O)Nc1ccc(C(=O)NC2C/C(=C\c3ccc(F)c(F)c3)C(=O)/C(=C/c3ccc(F)c(F)c3)C2)cc1]

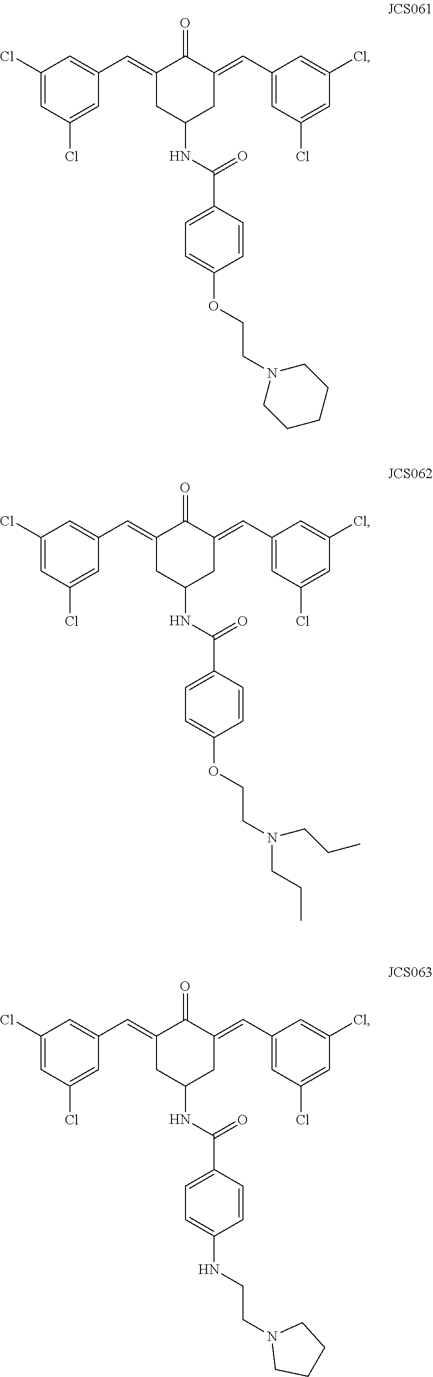 [SMILES: O=C1/C(=C/c2cc(Cl)cc(Cl)c2)CC(NC(=O)c2ccc(OCCN3CCCCC3)cc2)C/C1=C\c1cc(Cl)cc(Cl)c1 ; CCCN(CCC)CCOc1ccc(C(=O)NC2C/C(=C\c3cc(Cl)cc(Cl)c3)C(=O)/C(=C/c3cc(Cl)cc(Cl)c3)C2)cc1 ; O=C1/C(=C/c2cc(Cl)cc(Cl)c2)CC(NC(=O)c2ccc(NCCN3CCCC3)cc2)C/C1=C\c1cc(Cl)cc(Cl)c1]

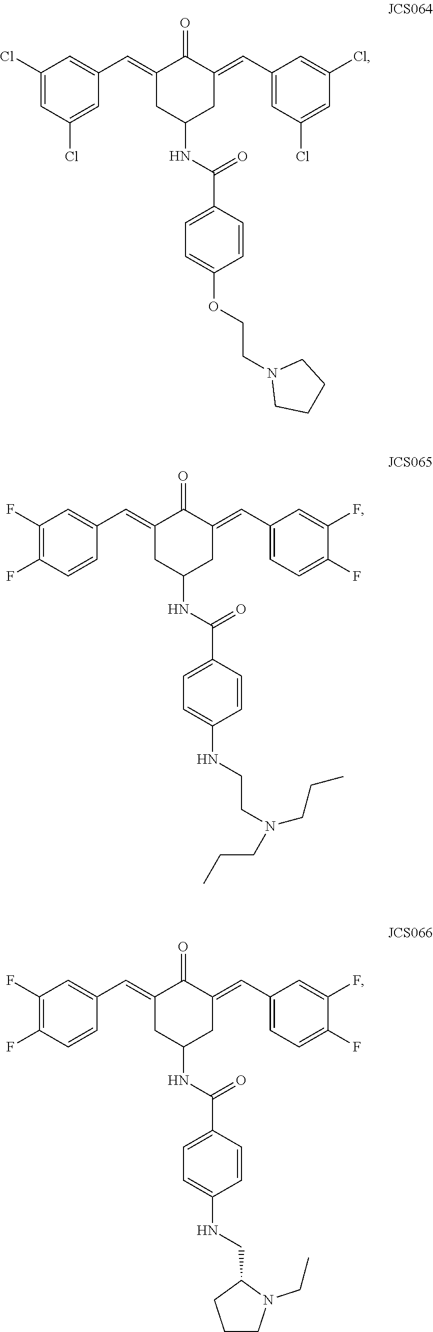 [SMILES: O=C1/C(=C/c2cc(Cl)cc(Cl)c2)CC(NC(=O)c2ccc(OCCN3CCCC3)cc2)C/C1=C\c1cc(Cl)cc(Cl)c1 ; CCCN(CCC)CCNc1ccc(C(=O)NC2C/C(=C\c3ccc(F)c(F)c3)C(=O)/C(=C/c3ccc(F)c(F)c3)C2)cc1 ; CCN1CCC[C@@H]1CNc1ccc(C(=O)NC2C/C(=C\c3ccc(F)c(F)c3)C(=O)/C(=C/c3ccc(F)c(F)c3)C2)cc1]

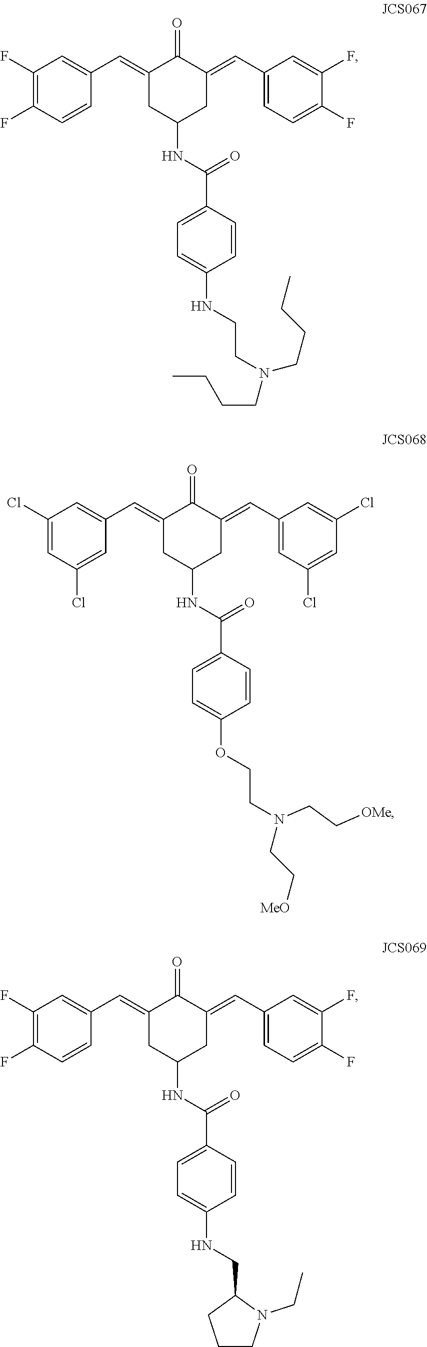 [SMILES: CCCCN(CCCC)CCNc1ccc(C(=O)NC2C/C(=C\c3ccc(F)c(F)c3)C(=O)/C(=C/c3ccc(F)c(F)c3)C2)cc1 ; COCCN(CCOC)CCOc1ccc(C(=O)NC2C/C(=C\c3cc(Cl)cc(Cl)c3)C(=O)/C(=C/c3cc(Cl)cc(Cl)c3)C2)cc1 ; CCN1CCC[C@H]1CNc1ccc(C(=O)NC2C/C(=C\c3ccc(F)c(F)c3)C(=O)/C(=C/c3ccc(F)c(F)c3)C2)cc1]

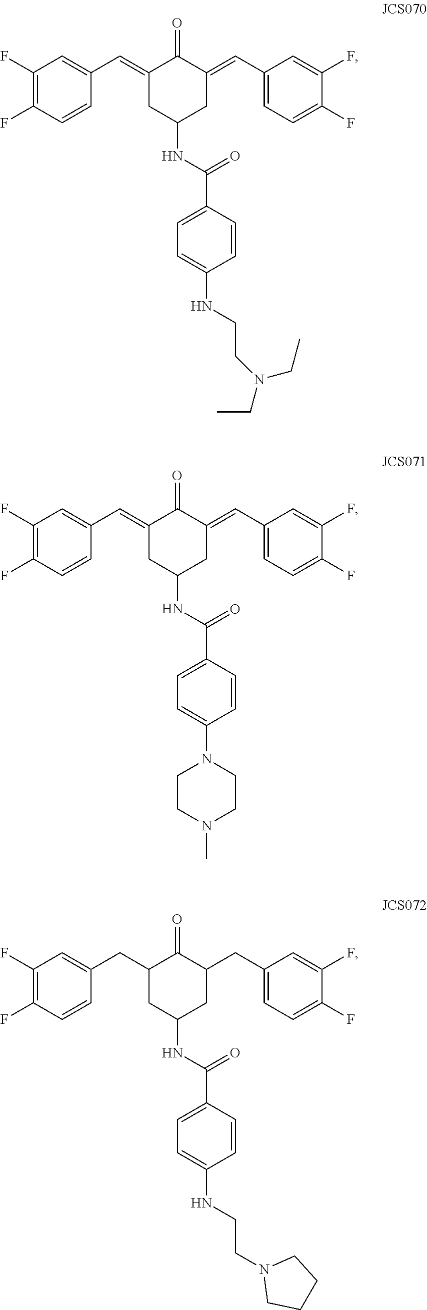 [SMILES: CCN(CC)CCNc1ccc(C(=O)NC2C/C(=C\c3ccc(F)c(F)c3)C(=O)/C(=C/c3ccc(F)c(F)c3)C2)cc1 ; CN1CCN(c2ccc(C(=O)NC3C/C(=C\c4ccc(F)c(F)c4)C(=O)/C(=C/c4ccc(F)c(F)c4)C3)cc2)CC1 ; O=C(NC1CC(Cc2ccc(F)c(F)c2)C(=O)C(Cc2ccc(F)c(F)c2)C1)c1ccc(NCCN2CCCC2)cc1]

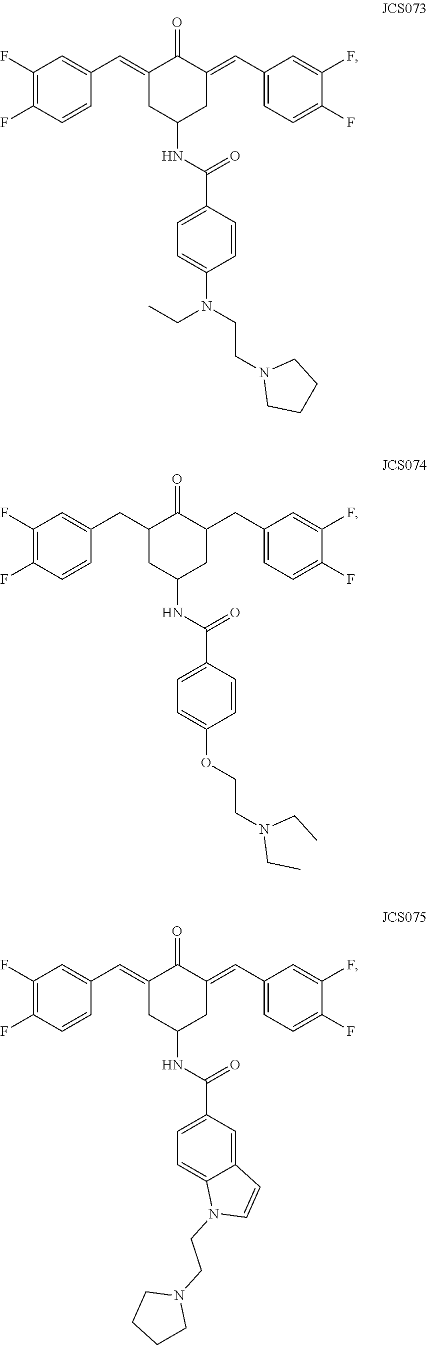 [SMILES: CCN(CCN1CCCC1)c1ccc(C(=O)NC2C/C(=C\c3ccc(F)c(F)c3)C(=O)/C(=C/c3ccc(F)c(F)c3)C2)cc1 ; CCN(CC)CCOc1ccc(C(=O)NC2CC(Cc3ccc(F)c(F)c3)C(=O)C(Cc3ccc(F)c(F)c3)C2)cc1 ; O=C1/C(=C/c2ccc(F)c(F)c2)CC(NC(=O)c2ccc3c(ccn3CCN3CCCC3)c2)C/C1=C\c1ccc(F)c(F)c1]

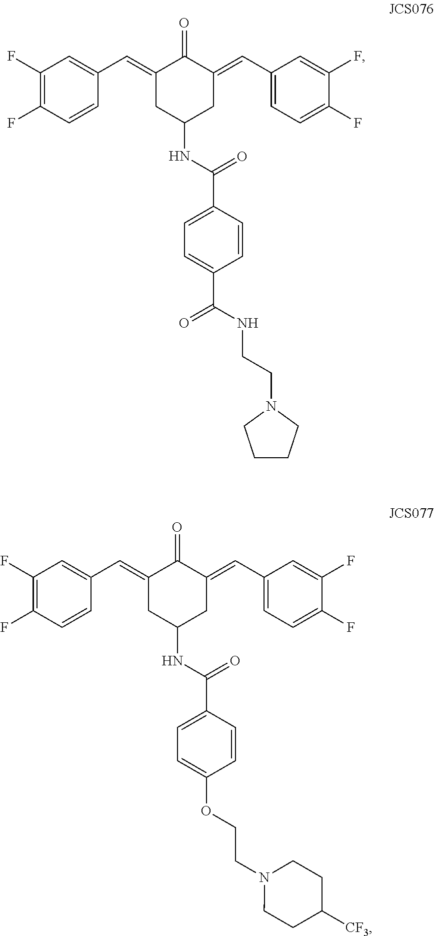 [SMILES: O=C1/C(=C/c2ccc(F)c(F)c2)CC(NC(=O)c2ccc(C(=O)NCCN3CCCC3)cc2)C/C1=C\c1ccc(F)c(F)c1 ; O=C1/C(=C/c2ccc(F)c(F)c2)CC(NC(=O)c2ccc(OCCN3CCC(C(F)(F)F)CC3)cc2)C/C1=C\c1ccc(F)c(F)c1]

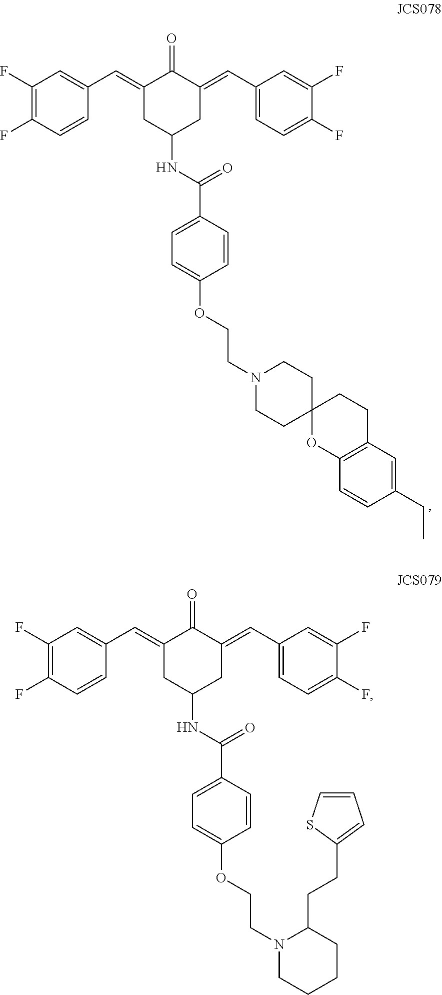 [SMILES: CCc1ccc2c(c1)CCC1(CCN(CCOc3ccc(C(=O)NC4C/C(=C\c5ccc(F)c(F)c5)C(=O)/C(=C/c5ccc(F)c(F)c5)C4)cc3)CC1)O2 ; O=C1/C(=C/c2ccc(F)c(F)c2)CC(NC(=O)c2ccc(OCCN3CCCCC3CCc3cccs3)cc2)C/C1=C\c1ccc(F)c(F)c1]

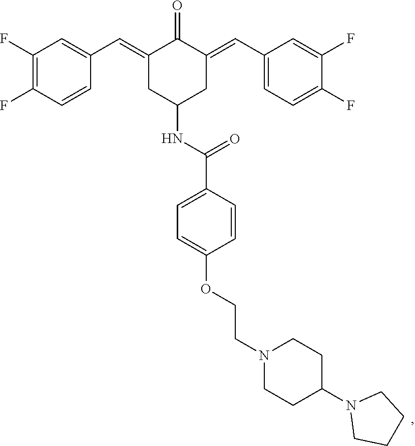 [SMILES: O=C1/C(=C/c2ccc(F)c(F)c2)CC(NC(=O)c2ccc(OCCN3CCC(N4CCCC4)CC3)cc2)C/C1=C\c1ccc(F)c(F)c1]

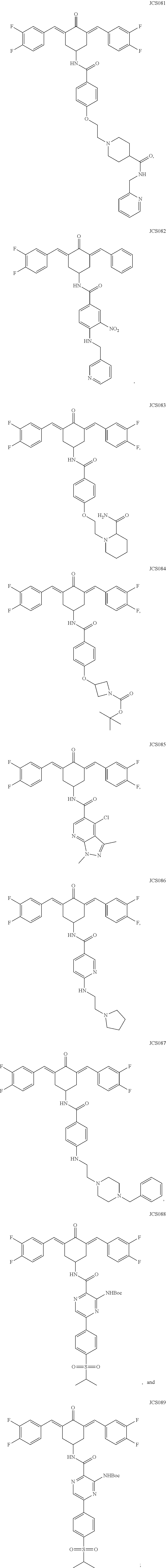

or a pharmaceutically acceptable salt thereof.

In certain embodiments, the compound is selected from

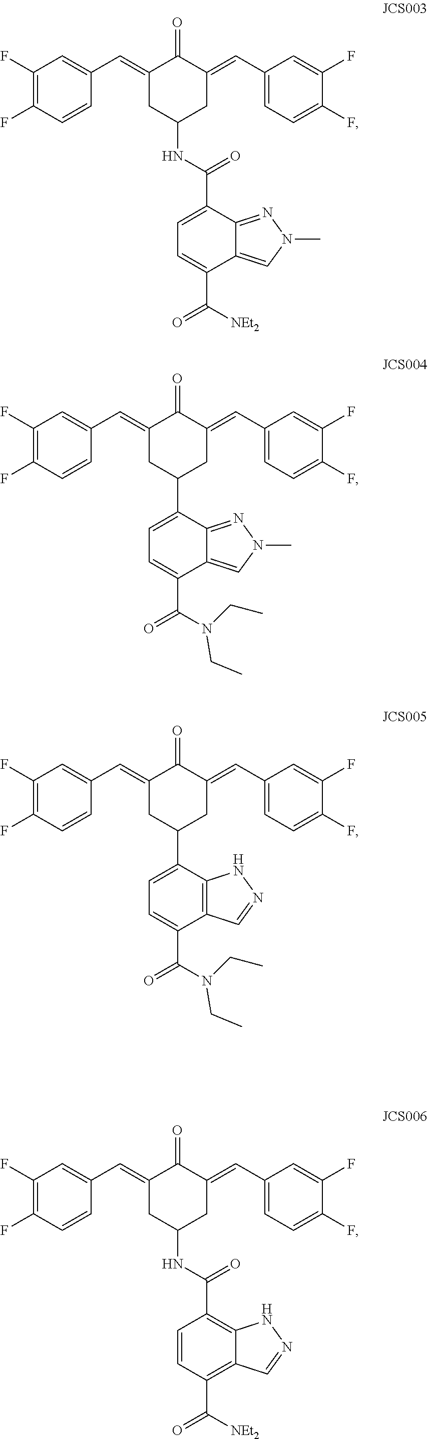

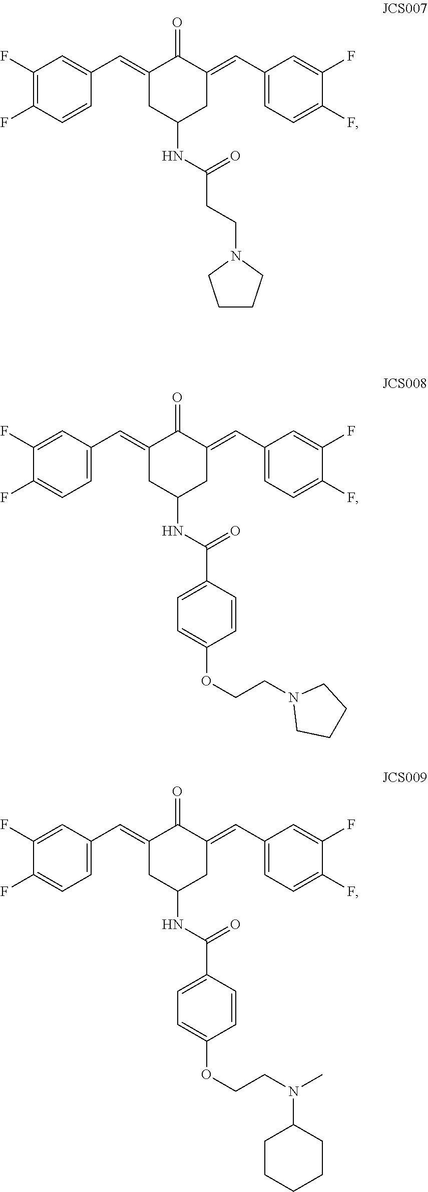 [SMILES: O=C(CCN1CCCC1)NC1C/C(=C\c2ccc(F)c(F)c2)C(=O)/C(=C/c2ccc(F)c(F)c2)C1 ; O=C1/C(=C/c2ccc(F)c(F)c2)CC(NC(=O)c2ccc(OCCN3CCCC3)cc2)C/C1=C\c1ccc(F)c(F)c1 ; CN(CCOc1ccc(C(=O)NC2C/C(=C\c3ccc(F)c(F)c3)C(=O)/C(=C/c3ccc(F)c(F)c3)C2)cc1)C1CCCCC1]

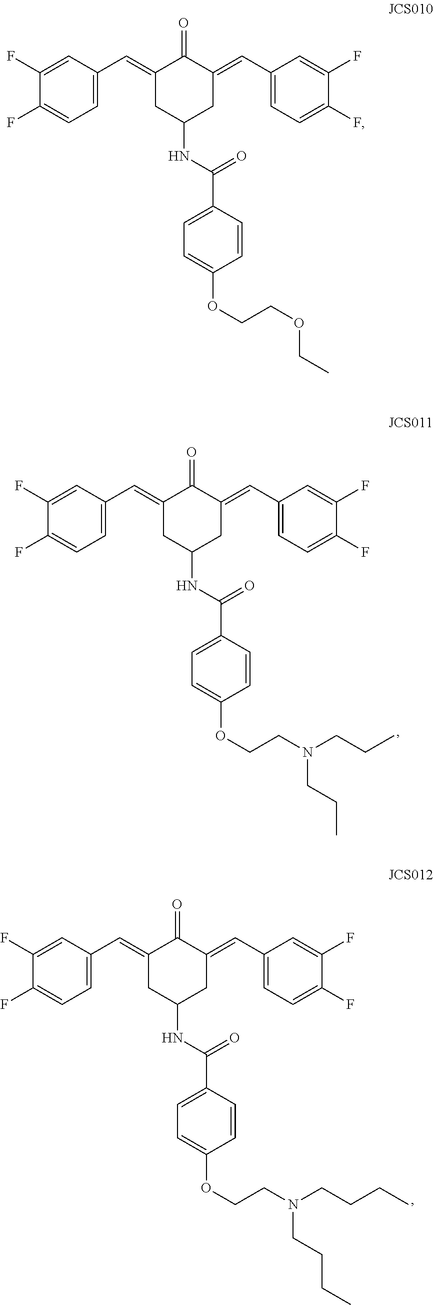 [SMILES: CCOCCOc1ccc(C(=O)NC2C/C(=C\c3ccc(F)c(F)c3)C(=O)/C(=C/c3ccc(F)c(F)c3)C2)cc1 ; CCCN(CCC)CCOc1ccc(C(=O)NC2C/C(=C\c3ccc(F)c(F)c3)C(=O)/C(=C/c3ccc(F)c(F)c3)C2)cc1 ; CCCCN(CCCC)CCOc1ccc(C(=O)NC2C/C(=C\c3ccc(F)c(F)c3)C(=O)/C(=C/c3ccc(F)c(F)c3)C2)cc1]

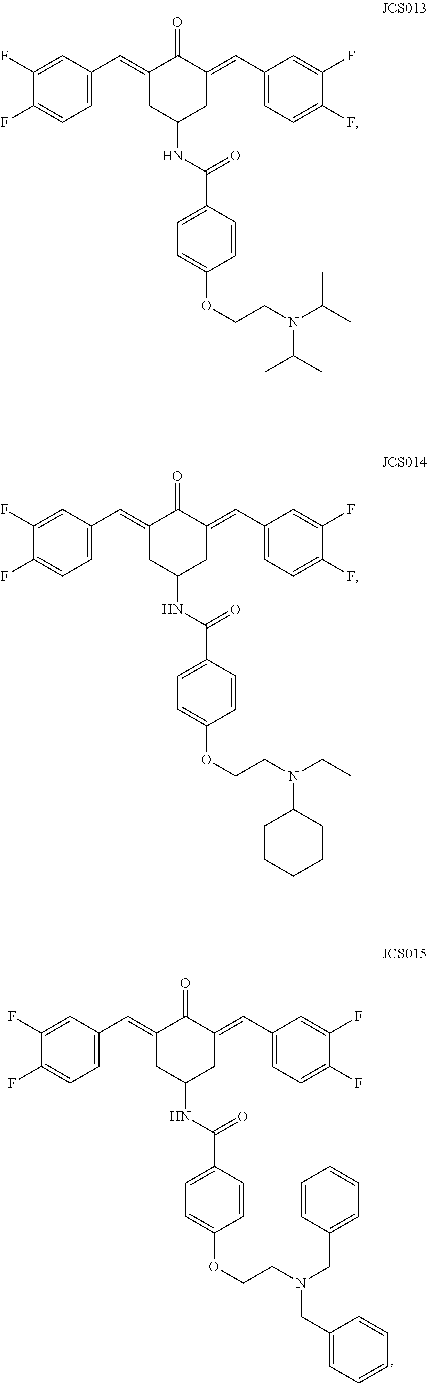 [SMILES: CC(C)N(CCOc1ccc(C(=O)NC2C/C(=C\c3ccc(F)c(F)c3)C(=O)/C(=C/c3ccc(F)c(F)c3)C2)cc1)C(C)C ; CCN(CCOc1ccc(C(=O)NC2C/C(=C\c3ccc(F)c(F)c3)C(=O)/C(=C/c3ccc(F)c(F)c3)C2)cc1)C1CCCCC1 ; O=C1/C(=C/c2ccc(F)c(F)c2)CC(NC(=O)c2ccc(OCCN(Cc3ccccc3)Cc3ccccc3)cc2)C/C1=C\c1ccc(F)c(F)c1]

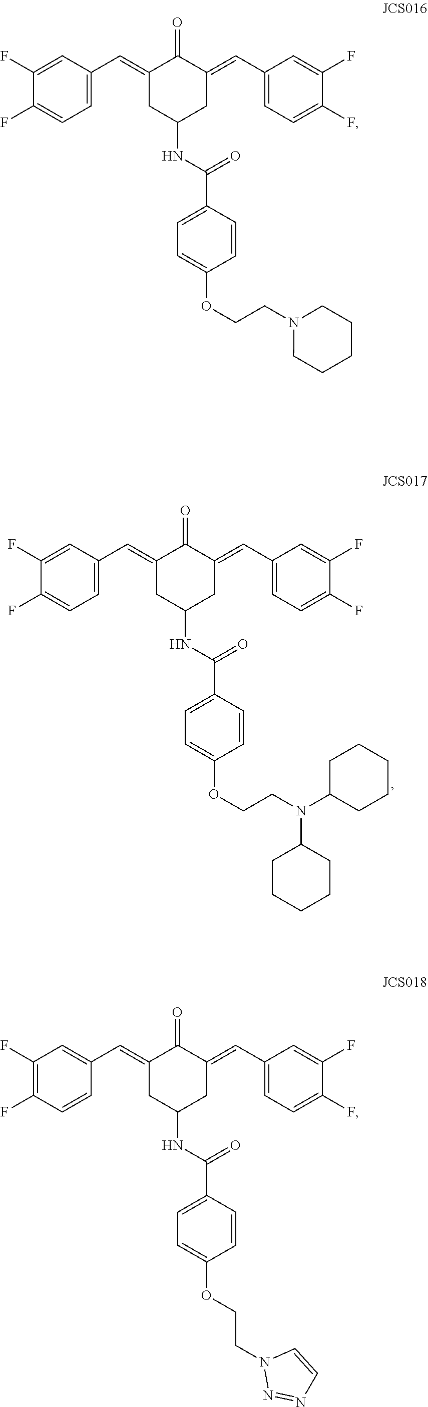 [SMILES: O=C1/C(=C/c2ccc(F)c(F)c2)CC(NC(=O)c2ccc(OCCN3CCCCC3)cc2)C/C1=C\c1ccc(F)c(F)c1 ; O=C1/C(=C/c2ccc(F)c(F)c2)CC(NC(=O)c2ccc(OCCN(C3CCCCC3)C3CCCCC3)cc2)C/C1=C\c1ccc(F)c(F)c1 ; O=C1/C(=C/c2ccc(F)c(F)c2)CC(NC(=O)c2ccc(OCCn3ccnn3)cc2)C/C1=C\c1ccc(F)c(F)c1]

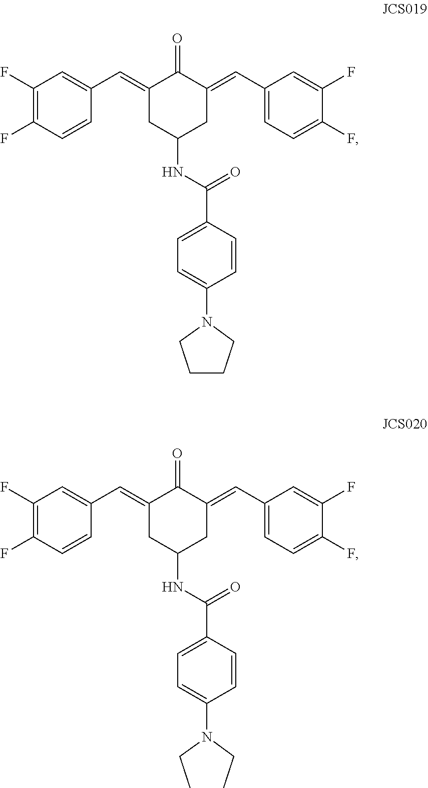 [SMILES: O=C1/C(=C/c2ccc(F)c(F)c2)CC(NC(=O)c2ccc(N3CCCC3)cc2)C/C1=C\c1ccc(F)c(F)c1 ; CCN(CC)c1ccc(C(=O)NC2C/C(=C\c3ccc(F)c(F)c3)C(=O)/C(=C/c3ccc(F)c(F)c3)C2)cc1]

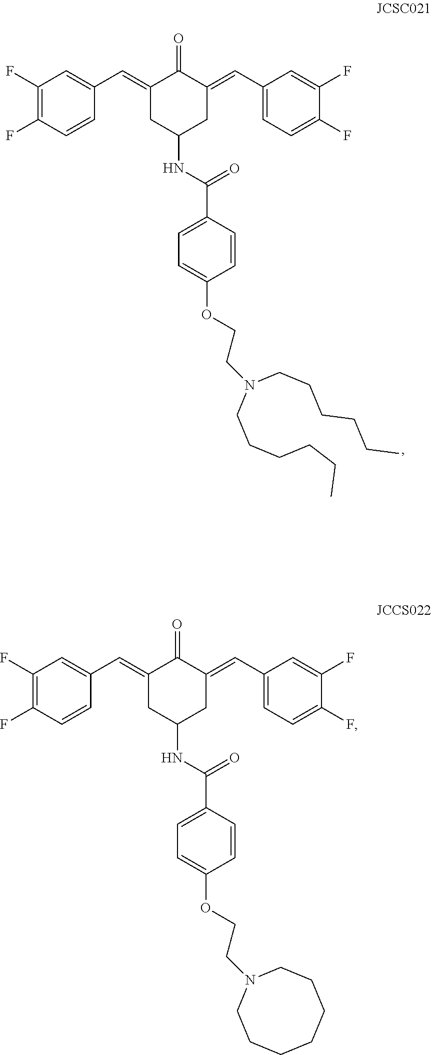 [SMILES: CCCCCCN(CCCCCC)CCOc1ccc(C(=O)NC2C/C(=C\c3ccc(F)c(F)c3)C(=O)/C(=C/c3ccc(F)c(F)c3)C2)cc1 ; O=C1/C(=C/c2ccc(F)c(F)c2)CC(NC(=O)c2ccc(OCCN3CCCCCCC3)cc2)C/C1=C\c1ccc(F)c(F)c1]

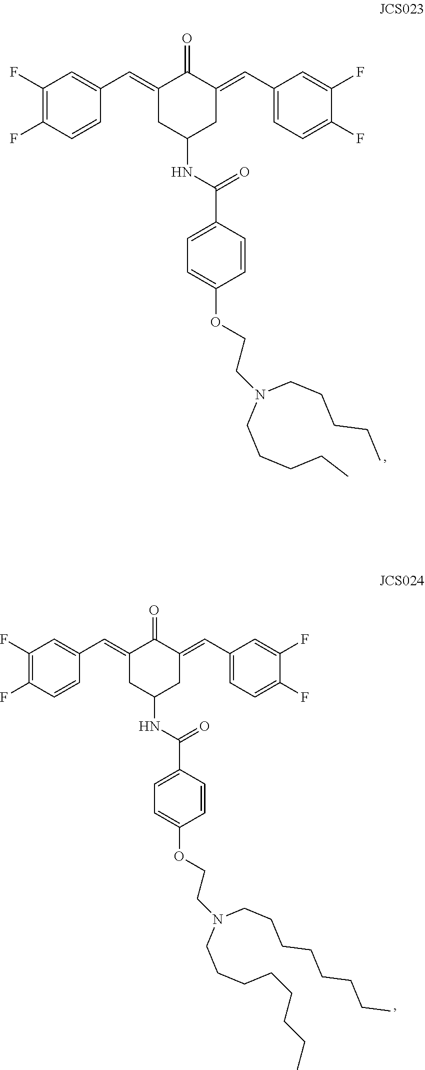 [SMILES: CCCCCN(CCCCC)CCOc1ccc(C(=O)NC2C/C(=C\c3ccc(F)c(F)c3)C(=O)/C(=C/c3ccc(F)c(F)c3)C2)cc1 ; CCCCCCCCN(CCCCCCCC)CCOc1ccc(C(=O)NC2C/C(=C\c3ccc(F)c(F)c3)C(=O)/C(=C/c3ccc(F)c(F)c3)C2)cc1]

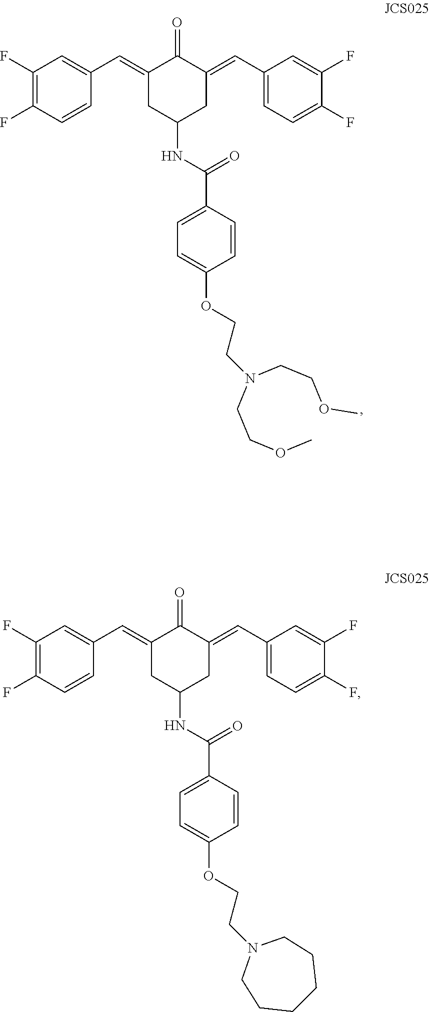 [SMILES: COCCN(CCOC)CCOc1ccc(C(=O)NC2C/C(=C\c3ccc(F)c(F)c3)C(=O)/C(=C/c3ccc(F)c(F)c3)C2)cc1 ; O=C1/C(=C/c2ccc(F)c(F)c2)CC(NC(=O)c2ccc(OCCN3CCCCCC3)cc2)C/C1=C\c1ccc(F)c(F)c1]

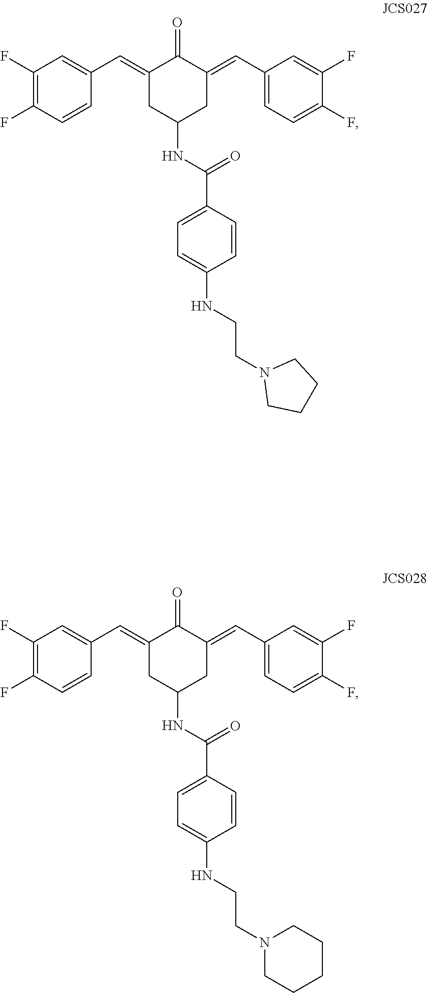 [SMILES: O=C1/C(=C/c2ccc(F)c(F)c2)CC(NC(=O)c2ccc(NCCN3CCCC3)cc2)C/C1=C\c1ccc(F)c(F)c1 ; O=C1/C(=C/c2ccc(F)c(F)c2)CC(NC(=O)c2ccc(NCCN3CCCCC3)cc2)C/C1=C\c1ccc(F)c(F)c1]

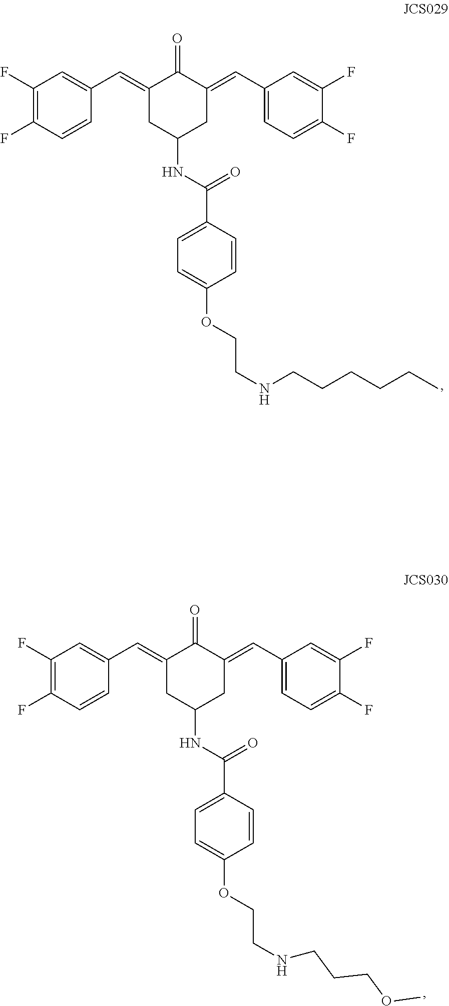 [SMILES: CCCCCCNCCOc1ccc(C(=O)NC2C/C(=C\c3ccc(F)c(F)c3)C(=O)/C(=C/c3ccc(F)c(F)c3)C2)cc1 ; COCCCNCCOc1ccc(C(=O)NC2C/C(=C\c3ccc(F)c(F)c3)C(=O)/C(=C/c3ccc(F)c(F)c3)C2)cc1]

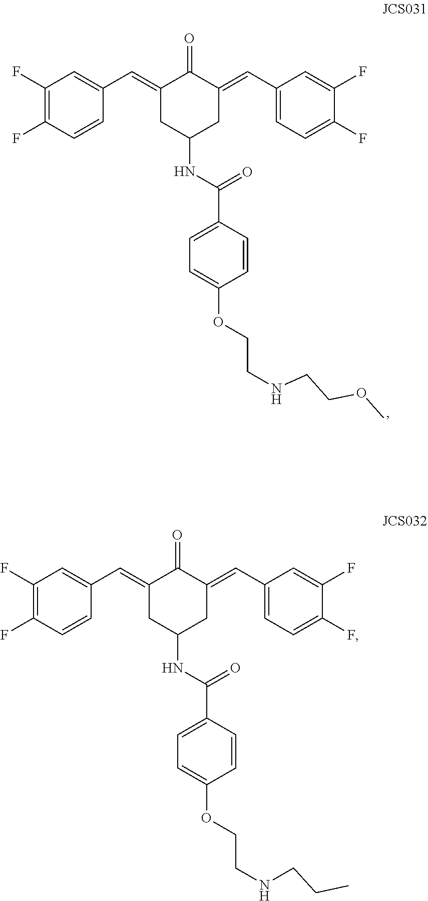 [SMILES: COCCNCCOc1ccc(C(=O)NC2C/C(=C\c3ccc(F)c(F)c3)C(=O)/C(=C/c3ccc(F)c(F)c3)C2)cc1 ; CCCNCCOc1ccc(C(=O)NC2C/C(=C\c3ccc(F)c(F)c3)C(=O)/C(=C/c3ccc(F)c(F)c3)C2)cc1]

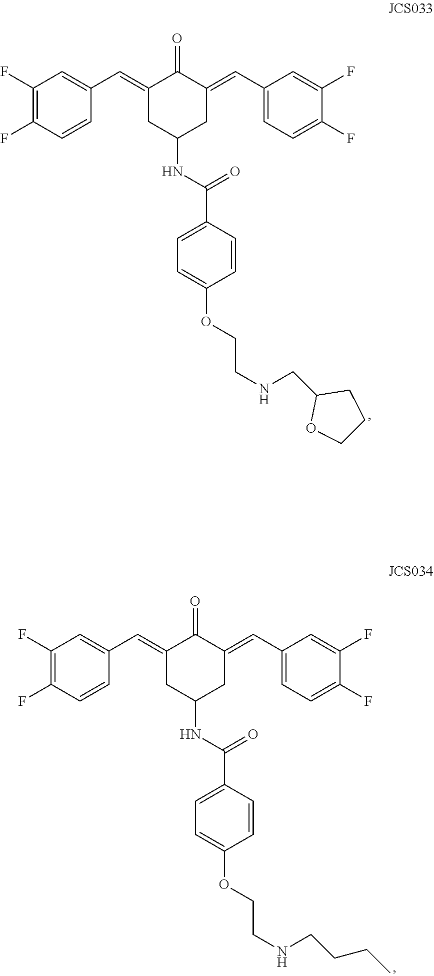 [SMILES: O=C1/C(=C/c2ccc(F)c(F)c2)CC(NC(=O)c2ccc(OCCNCC3CCCO3)cc2)C/C1=C\c1ccc(F)c(F)c1 ; CCCCNCCOc1ccc(C(=O)NC2C/C(=C\c3ccc(F)c(F)c3)C(=O)/C(=C/c3ccc(F)c(F)c3)C2)cc1]

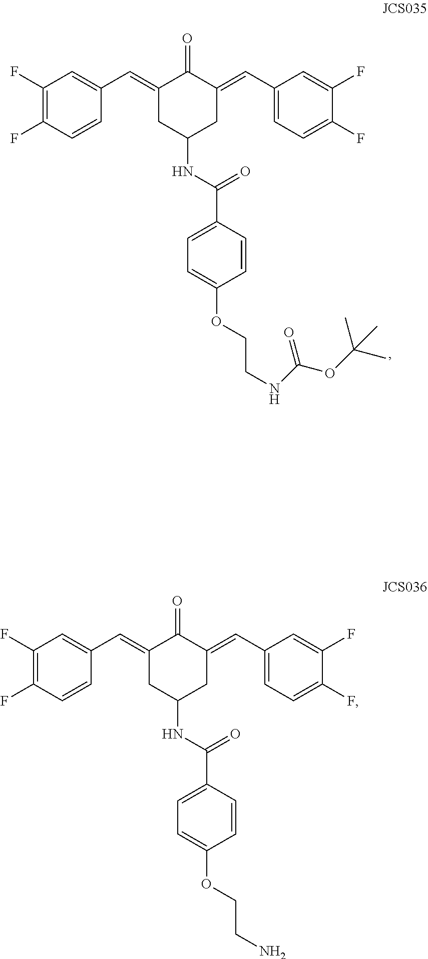 [SMILES: CC(C)(C)OC(=O)NCCOc1ccc(C(=O)NC2C/C(=C\c3ccc(F)c(F)c3)C(=O)/C(=C/c3ccc(F)c(F)c3)C2)cc1 ; NCCOc1ccc(C(=O)NC2C/C(=C\c3ccc(F)c(F)c3)C(=O)/C(=C/c3ccc(F)c(F)c3)C2)cc1]

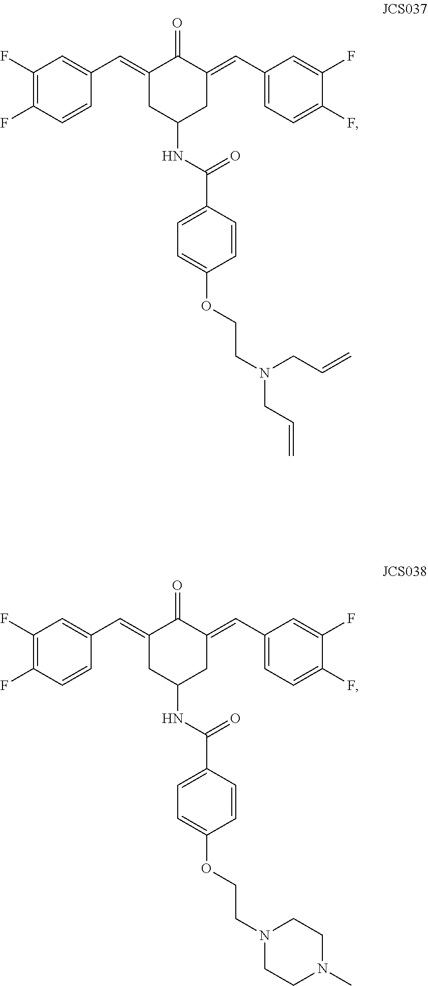 [SMILES: C=CCN(CC=C)CCOc1ccc(C(=O)NC2C/C(=C\c3ccc(F)c(F)c3)C(=O)/C(=C/c3ccc(F)c(F)c3)C2)cc1 ; CN1CCN(CCOc2ccc(C(=O)NC3C/C(=C\c4ccc(F)c(F)c4)C(=O)/C(=C/c4ccc(F)c(F)c4)C3)cc2)CC1]

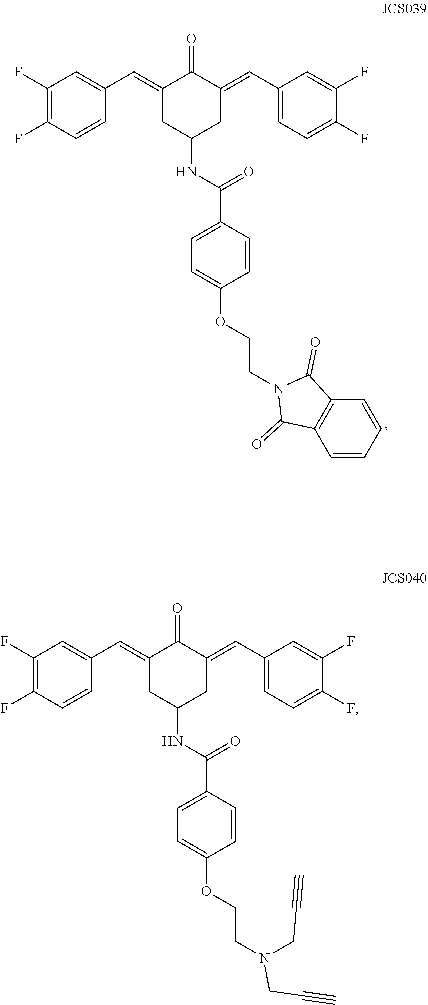 [SMILES: O=C1/C(=C/c2ccc(F)c(F)c2)CC(NC(=O)c2ccc(OCCN3C(=O)c4ccccc4C3=O)cc2)C/C1=C\c1ccc(F)c(F)c1 ; C#CCN(CC#C)CCOc1ccc(C(=O)NC2C/C(=C\c3ccc(F)c(F)c3)C(=O)/C(=C/c3ccc(F)c(F)c3)C2)cc1]

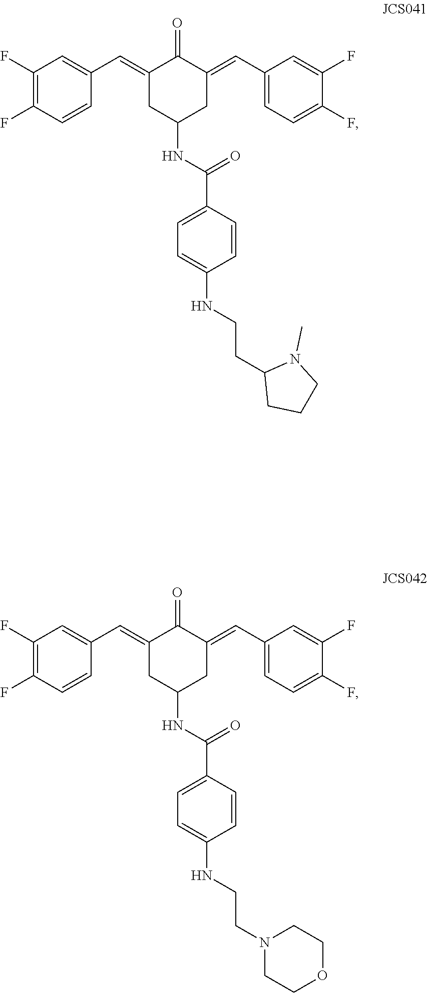 [SMILES: CN1CCCC1CCNc1ccc(C(=O)NC2C/C(=C\c3ccc(F)c(F)c3)C(=O)/C(=C/c3ccc(F)c(F)c3)C2)cc1 ; O=C1/C(=C/c2ccc(F)c(F)c2)CC(NC(=O)c2ccc(NCCN3CCOCC3)cc2)C/C1=C\c1ccc(F)c(F)c1]

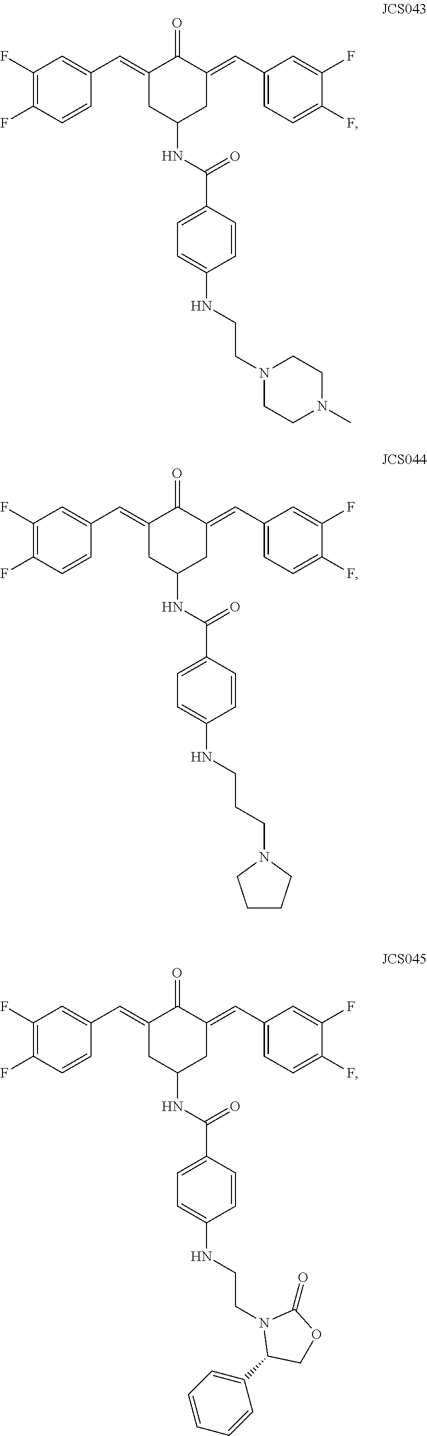 [SMILES: CN1CCN(CCNc2ccc(C(=O)NC3C/C(=C\c4ccc(F)c(F)c4)C(=O)/C(=C/c4ccc(F)c(F)c4)C3)cc2)CC1 ; O=C1/C(=C/c2ccc(F)c(F)c2)CC(NC(=O)c2ccc(NCCCN3CCCC3)cc2)C/C1=C\c1ccc(F)c(F)c1 ; O=C1/C(=C/c2ccc(F)c(F)c2)CC(NC(=O)c2ccc(NCCN3C(=O)OC[C@@H]3c3ccccc3)cc2)C/C1=C\c1ccc(F)c(F)c1]

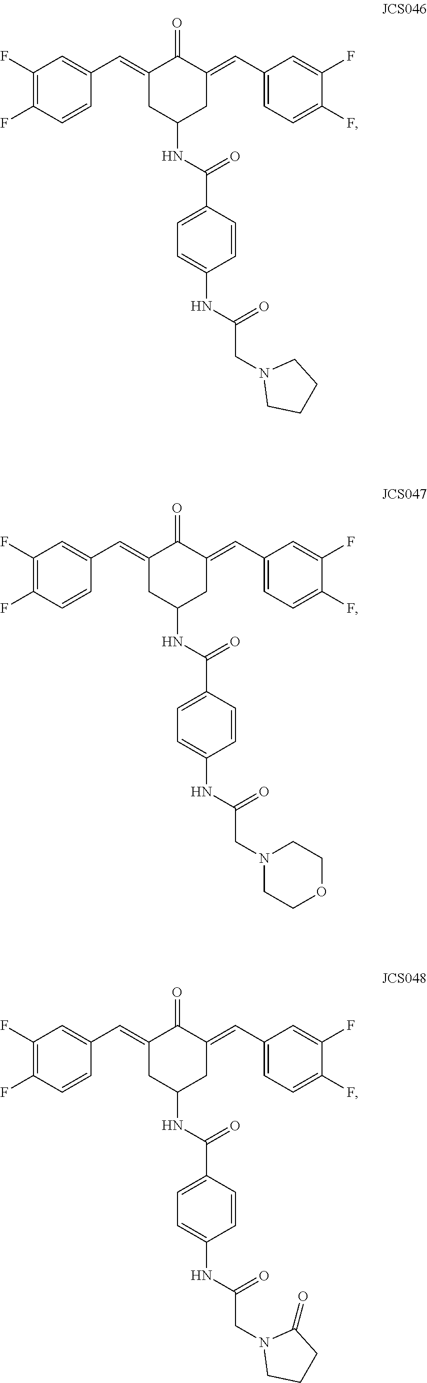 [SMILES: O=C(CN1CCCC1)Nc1ccc(C(=O)NC2C/C(=C\c3ccc(F)c(F)c3)C(=O)/C(=C/c3ccc(F)c(F)c3)C2)cc1 ; O=C(CN1CCOCC1)Nc1ccc(C(=O)NC2C/C(=C\c3ccc(F)c(F)c3)C(=O)/C(=C/c3ccc(F)c(F)c3)C2)cc1 ; O=C(CN1CCCC1=O)Nc1ccc(C(=O)NC2C/C(=C\c3ccc(F)c(F)c3)C(=O)/C(=C/c3ccc(F)c(F)c3)C2)cc1]

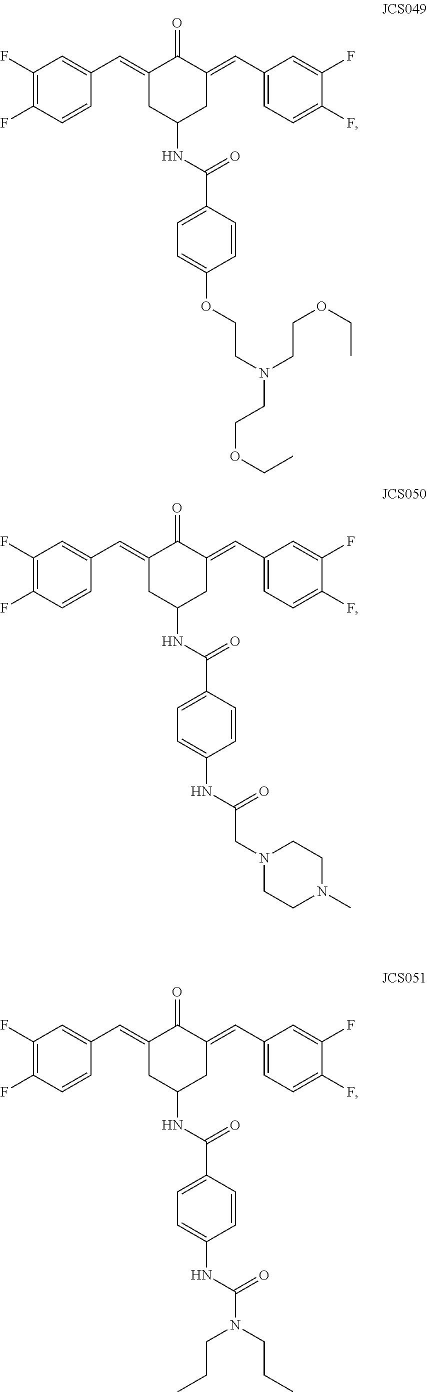 [SMILES: CCOCCN(CCOCC)CCOc1ccc(C(=O)NC2C/C(=C\c3ccc(F)c(F)c3)C(=O)/C(=C/c3ccc(F)c(F)c3)C2)cc1 ; CN1CCN(CC(=O)Nc2ccc(C(=O)NC3C/C(=C\c4ccc(F)c(F)c4)C(=O)/C(=C/c4ccc(F)c(F)c4)C3)cc2)CC1 ; CCCN(CCC)C(=O)Nc1ccc(C(=O)NC2C/C(=C\c3ccc(F)c(F)c3)C(=O)/C(=C/c3ccc(F)c(F)c3)C2)cc1]

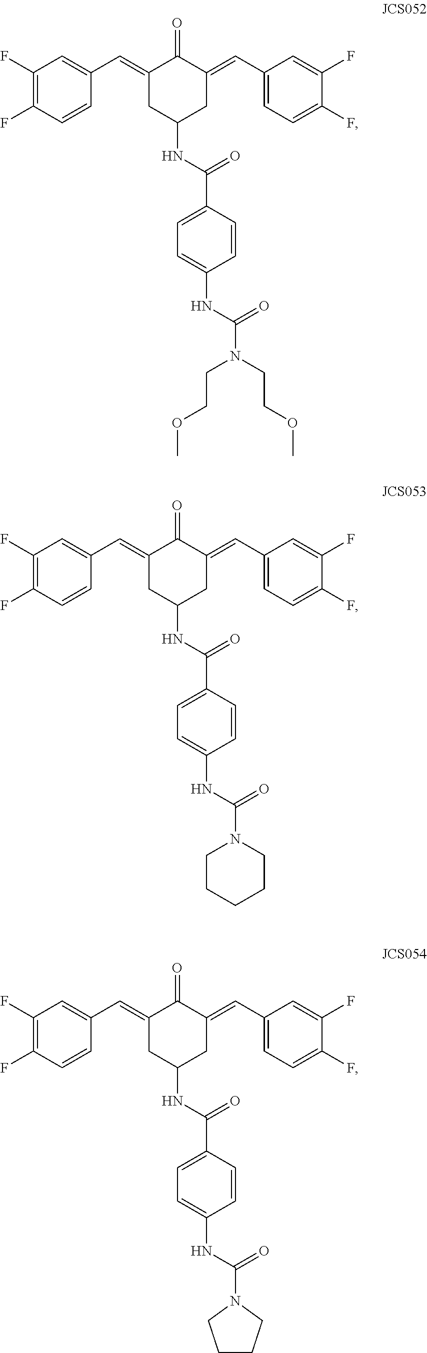 [SMILES: COCCN(CCOC)C(=O)Nc1ccc(C(=O)NC2C/C(=C\c3ccc(F)c(F)c3)C(=O)/C(=C/c3ccc(F)c(F)c3)C2)cc1 ; O=C1/C(=C/c2ccc(F)c(F)c2)CC(NC(=O)c2ccc(NC(=O)N3CCCCC3)cc2)C/C1=C\c1ccc(F)c(F)c1 ; O=C1/C(=C/c2ccc(F)c(F)c2)CC(NC(=O)c2ccc(NC(=O)N3CCCC3)cc2)C/C1=C\c1ccc(F)c(F)c1]

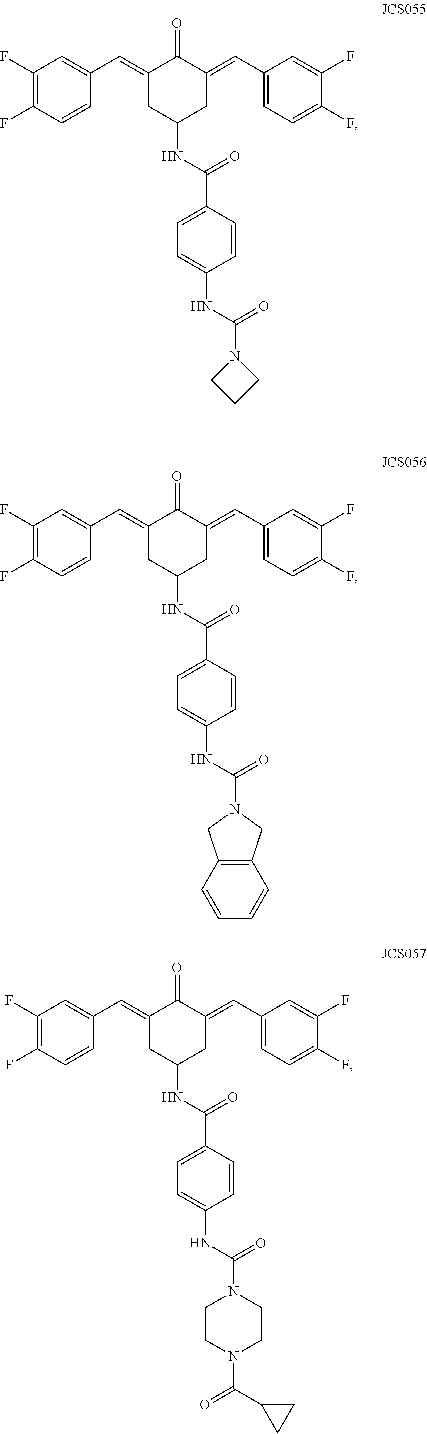 [SMILES: O=C1/C(=C/c2ccc(F)c(F)c2)CC(NC(=O)c2ccc(NC(=O)N3CCC3)cc2)C/C1=C\c1ccc(F)c(F)c1 ; O=C1/C(=C/c2ccc(F)c(F)c2)CC(NC(=O)c2ccc(NC(=O)N3Cc4ccccc4C3)cc2)C/C1=C\c1ccc(F)c(F)c1 ; O=C1/C(=C/c2ccc(F)c(F)c2)CC(NC(=O)c2ccc(NC(=O)N3CCN(C(=O)C4CC4)CC3)cc2)C/C1=C\c1ccc(F)c(F)c1]

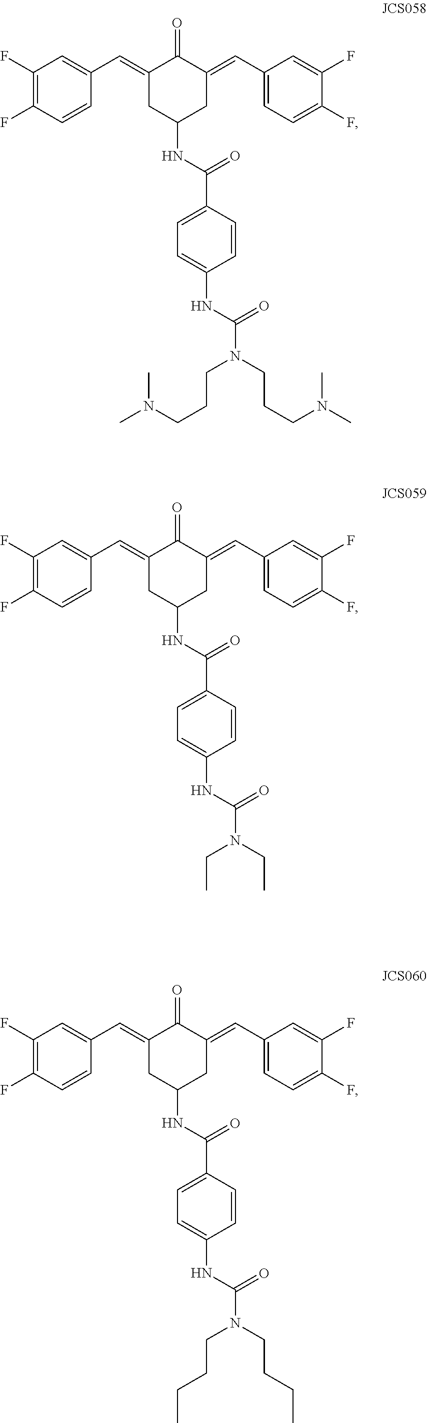 [SMILES: CN(C)CCCN(CCCN(C)C)C(=O)Nc1ccc(C(=O)NC2C/C(=C\c3ccc(F)c(F)c3)C(=O)/C(=C/c3ccc(F)c(F)c3)C2)cc1 ; CCN(CC)C(=O)Nc1ccc(C(=O)NC2C/C(=C\c3ccc(F)c(F)c3)C(=O)/C(=C/c3ccc(F)c(F)c3)C2)cc1 ; CCCCN(CCCC)C(=O)Nc1ccc(C(=O)NC2C/C(=C\c3ccc(F)c(F)c3)C(=O)/C(=C/c3ccc(F)c(F)c3)C2)cc1]

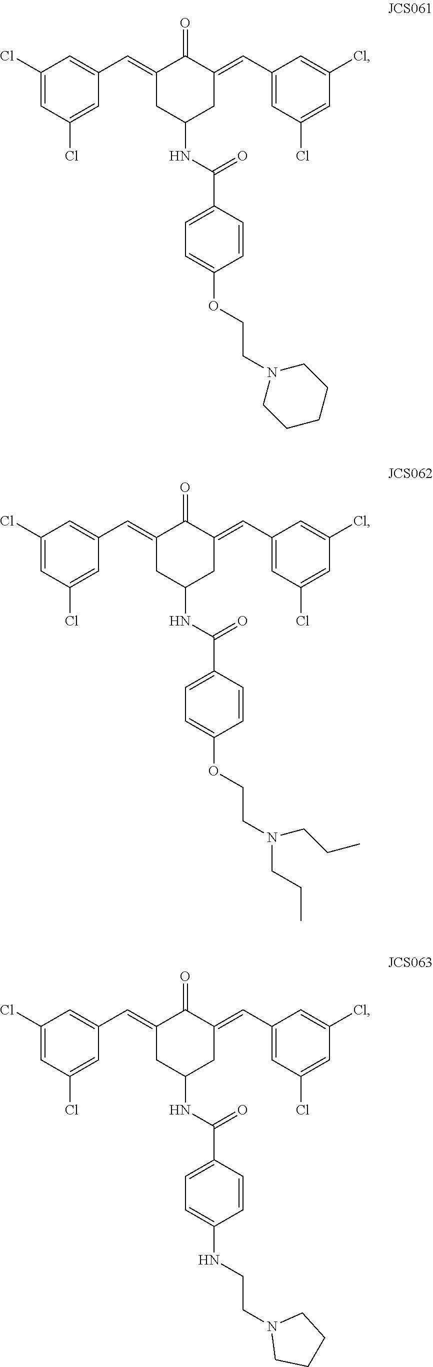 [SMILES: O=C1/C(=C/c2cc(Cl)cc(Cl)c2)CC(NC(=O)c2ccc(OCCN3CCCCC3)cc2)C/C1=C\c1cc(Cl)cc(Cl)c1 ; CCCN(CCC)CCOc1ccc(C(=O)NC2C/C(=C\c3cc(Cl)cc(Cl)c3)C(=O)/C(=C/c3cc(Cl)cc(Cl)c3)C2)cc1 ; O=C1/C(=C/c2cc(Cl)cc(Cl)c2)CC(NC(=O)c2ccc(NCCN3CCCC3)cc2)C/C1=C\c1cc(Cl)cc(Cl)c1]

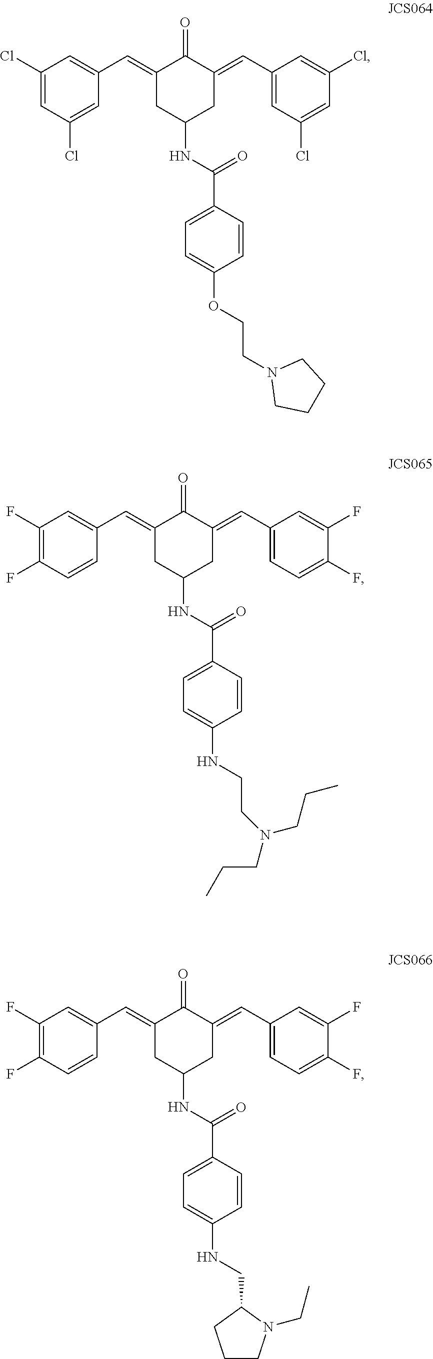 [SMILES: O=C1/C(=C/c2cc(Cl)cc(Cl)c2)CC(NC(=O)c2ccc(OCCN3CCCC3)cc2)C/C1=C\c1cc(Cl)cc(Cl)c1 ; CCCN(CCC)CCNc1ccc(C(=O)NC2C/C(=C\c3ccc(F)c(F)c3)C(=O)/C(=C/c3ccc(F)c(F)c3)C2)cc1 ; CCN1CCC[C@@H]1CNc1ccc(C(=O)NC2C/C(=C\c3ccc(F)c(F)c3)C(=O)/C(=C/c3ccc(F)c(F)c3)C2)cc1]

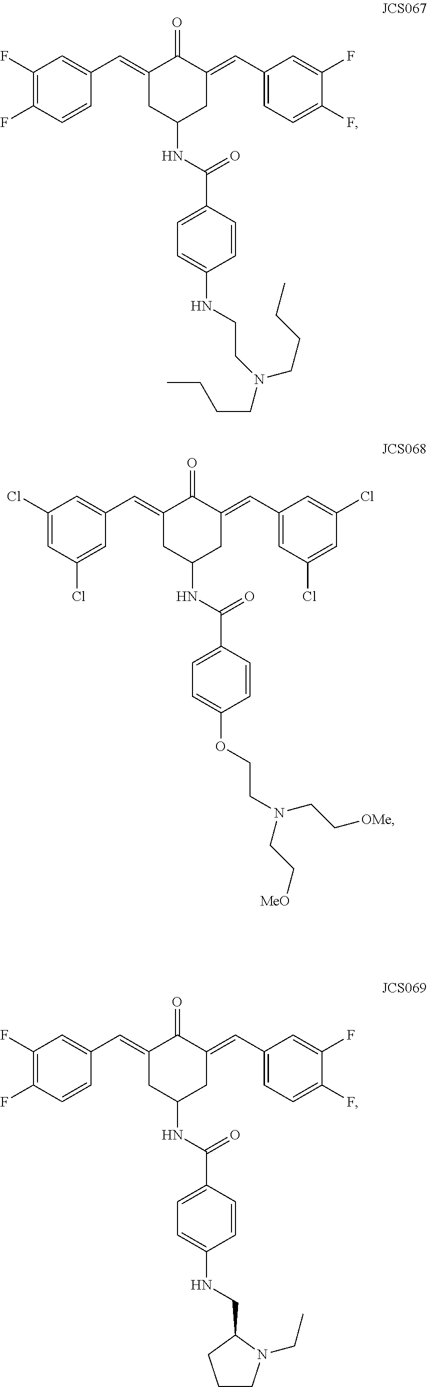 [SMILES: CCCCN(CCCC)CCNc1ccc(C(=O)NC2C/C(=C\c3ccc(F)c(F)c3)C(=O)/C(=C/c3ccc(F)c(F)c3)C2)cc1 ; COCCN(CCOC)CCOc1ccc(C(=O)NC2C/C(=C\c3cc(Cl)cc(Cl)c3)C(=O)/C(=C/c3cc(Cl)cc(Cl)c3)C2)cc1 ; CCN1CCC[C@H]1CNc1ccc(C(=O)NC2C/C(=C\c3ccc(F)c(F)c3)C(=O)/C(=C/c3ccc(F)c(F)c3)C2)cc1]

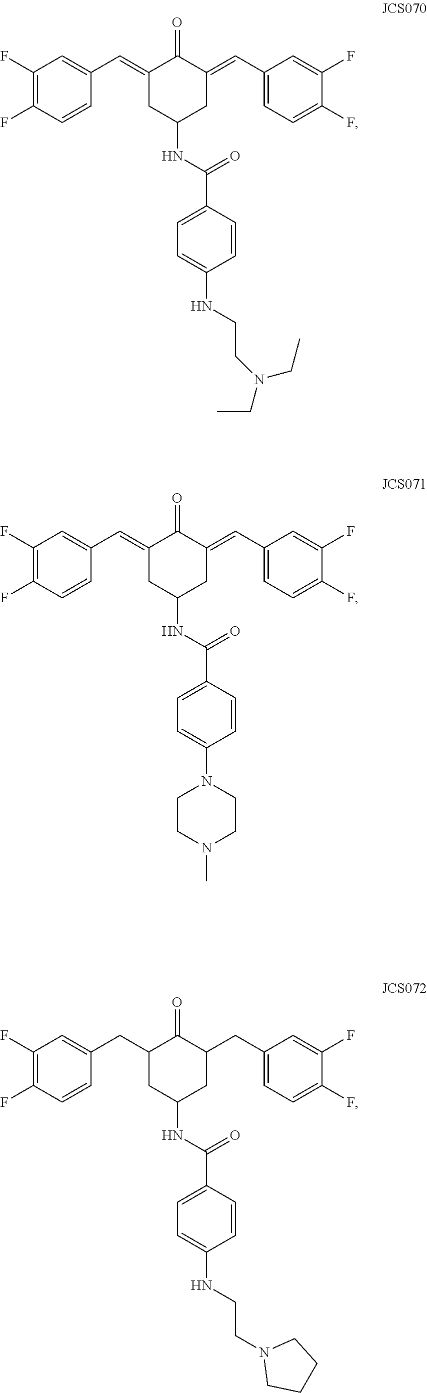 [SMILES: CCN(CC)CCNc1ccc(C(=O)NC2C/C(=C\c3ccc(F)c(F)c3)C(=O)/C(=C/c3ccc(F)c(F)c3)C2)cc1 ; CN1CCN(c2ccc(C(=O)NC3C/C(=C\c4ccc(F)c(F)c4)C(=O)/C(=C/c4ccc(F)c(F)c4)C3)cc2)CC1 ; O=C(NC1CC(Cc2ccc(F)c(F)c2)C(=O)C(Cc2ccc(F)c(F)c2)C1)c1ccc(NCCN2CCCC2)cc1]

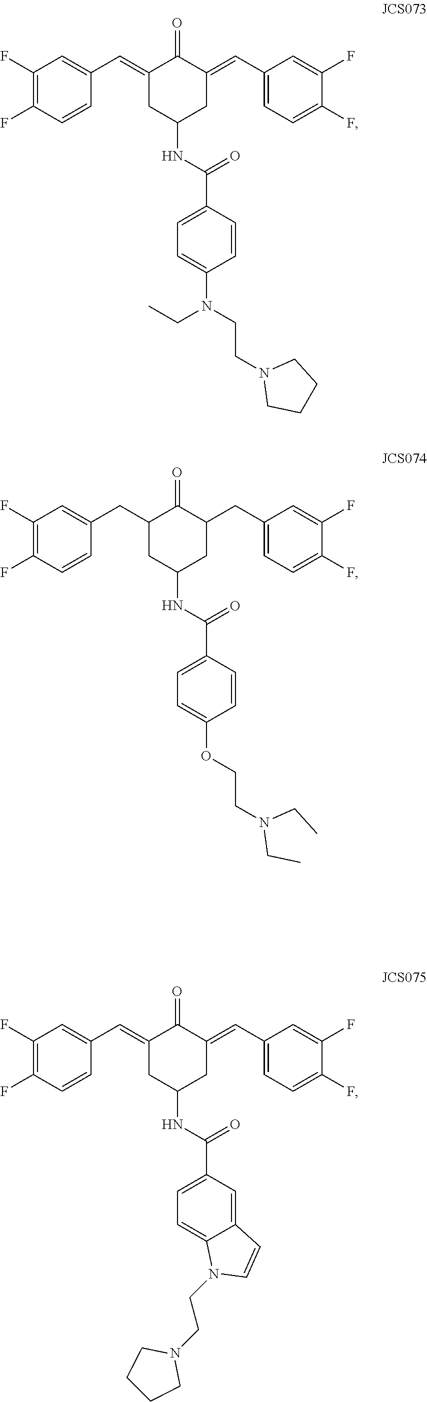 [SMILES: CCN(CCN1CCCC1)c1ccc(C(=O)NC2C/C(=C\c3ccc(F)c(F)c3)C(=O)/C(=C/c3ccc(F)c(F)c3)C2)cc1 ; CCN(CC)CCOc1ccc(C(=O)NC2CC(Cc3ccc(F)c(F)c3)C(=O)C(Cc3ccc(F)c(F)c3)C2)cc1 ; O=C1/C(=C/c2ccc(F)c(F)c2)CC(NC(=O)c2ccc3c(ccn3CCN3CCCC3)c2)C/C1=C\c1ccc(F)c(F)c1]

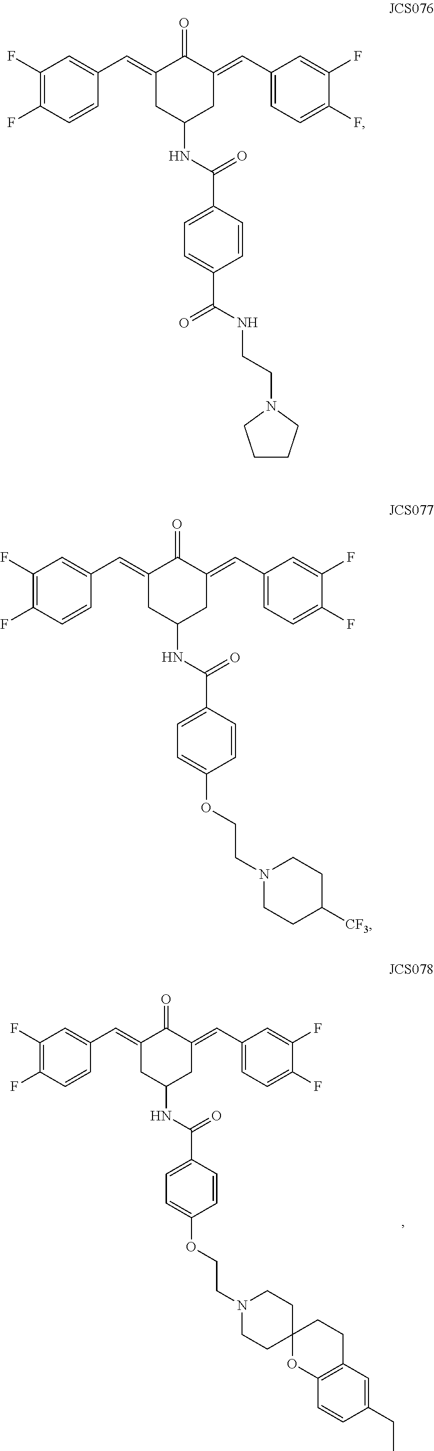 [SMILES: O=C1/C(=C/c2ccc(F)c(F)c2)CC(NC(=O)c2ccc(C(=O)NCCN3CCCC3)cc2)C/C1=C\c1ccc(F)c(F)c1 ; O=C1/C(=C/c2ccc(F)c(F)c2)CC(NC(=O)c2ccc(OCCN3CCC(C(F)(F)F)CC3)cc2)C/C1=C\c1ccc(F)c(F)c1 ; CCc1ccc2c(c1)CCC1(CCN(CCOc3ccc(C(=O)NC4C/C(=C\c5ccc(F)c(F)c5)C(=O)/C(=C/c5ccc(F)c(F)c5)C4)cc3)CC1)O2]

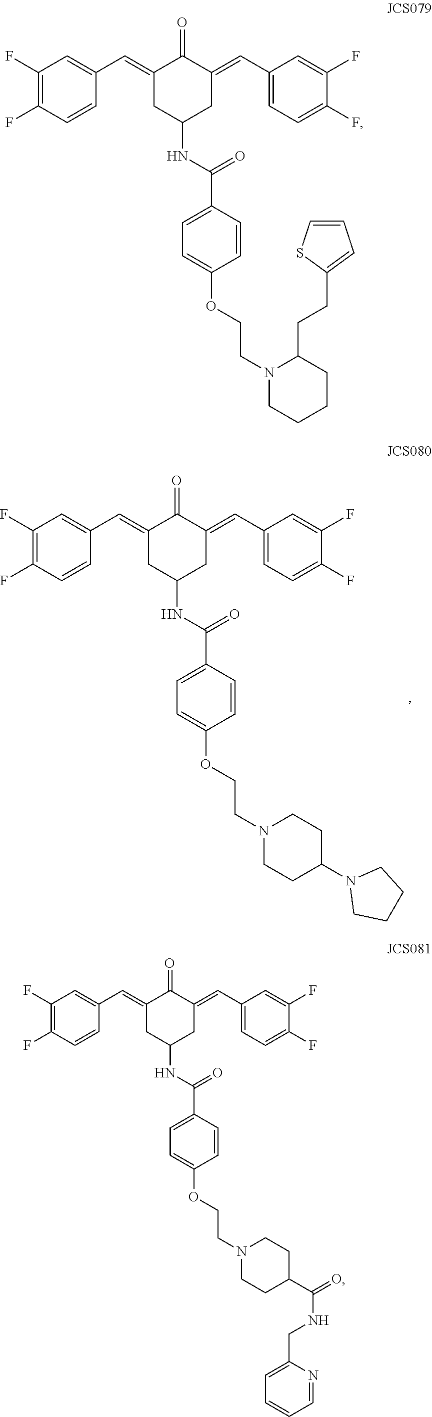 [SMILES: O=C1/C(=C/c2ccc(F)c(F)c2)CC(NC(=O)c2ccc(OCCN3CCCCC3CCc3cccs3)cc2)C/C1=C\c1ccc(F)c(F)c1 ; O=C1/C(=C/c2ccc(F)c(F)c2)CC(NC(=O)c2ccc(OCCN3CCC(N4CCCC4)CC3)cc2)C/C1=C\c1ccc(F)c(F)c1 ; O=C1/C(=C/c2ccc(F)c(F)c2)CC(NC(=O)c2ccc(OCCN3CCC(C(=O)NCc4ccccn4)CC3)cc2)C/C1=C\c1ccc(F)c(F)c1]

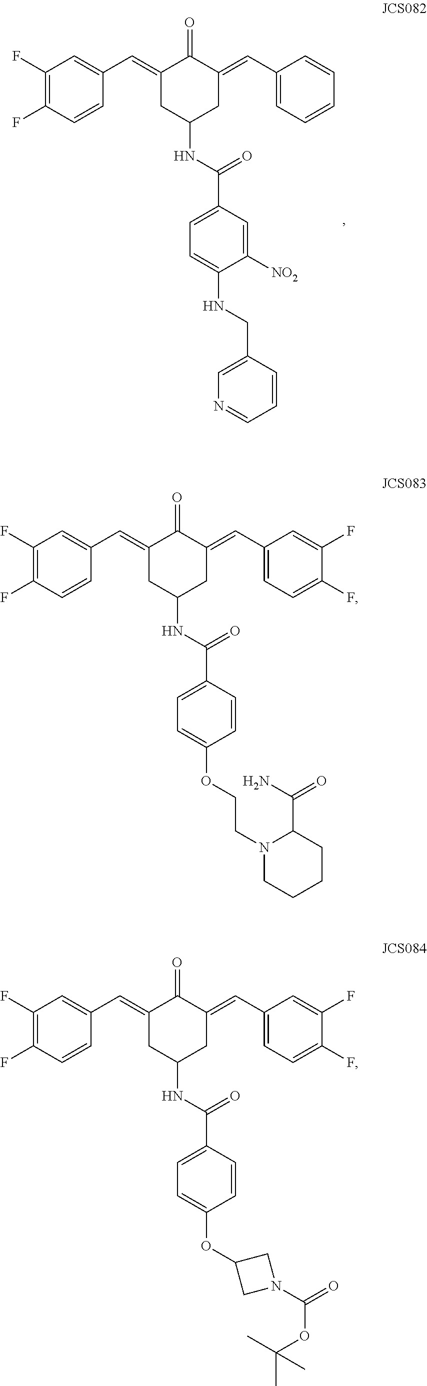 [SMILES: O=C1/C(=C/c2ccccc2)CC(NC(=O)c2ccc(NCc3cccnc3)c([N+](=O)[O-])c2)C/C1=C\c1ccc(F)c(F)c1 ; NC(=O)C1CCCCN1CCOc1ccc(C(=O)NC2C/C(=C\c3ccc(F)c(F)c3)C(=O)/C(=C/c3ccc(F)c(F)c3)C2)cc1 ; CC(C)(C)OC(=O)N1CC(Oc2ccc(C(=O)NC3C/C(=C\c4ccc(F)c(F)c4)C(=O)/C(=C/c4ccc(F)c(F)c4)C3)cc2)C1]

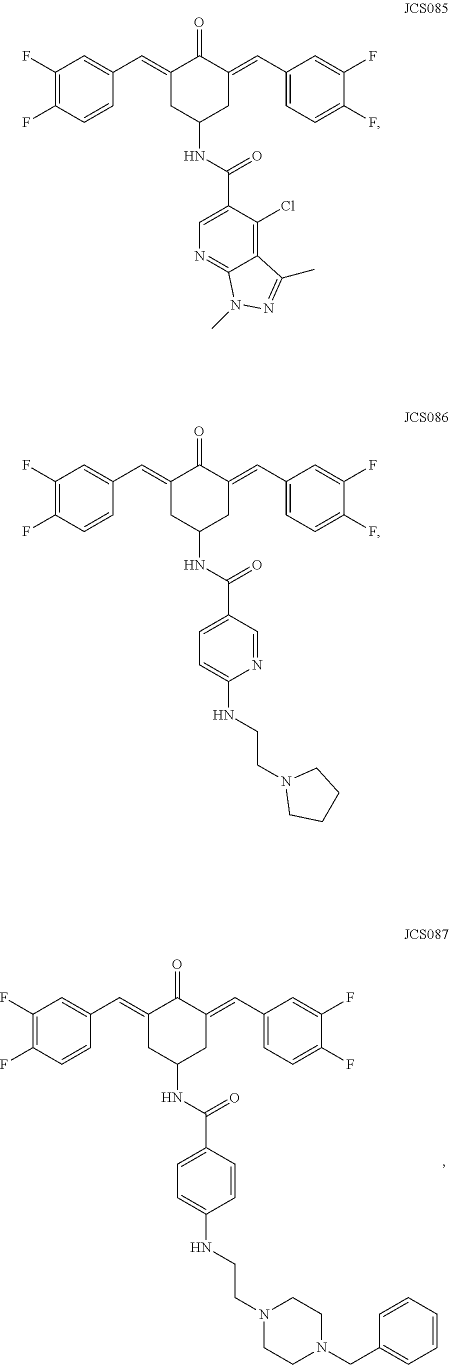 [SMILES: Cc1nn(C)c2ncc(C(=O)NC3C/C(=C\c4ccc(F)c(F)c4)C(=O)/C(=C/c4ccc(F)c(F)c4)C3)c(Cl)c12 ; O=C1/C(=C/c2ccc(F)c(F)c2)CC(NC(=O)c2ccc(NCCN3CCCC3)nc2)C/C1=C\c1ccc(F)c(F)c1 ; O=C1/C(=C/c2ccc(F)c(F)c2)CC(NC(=O)c2ccc(NCCN3CCN(Cc4ccccc4)CC3)cc2)C/C1=C\c1ccc(F)c(F)c1]

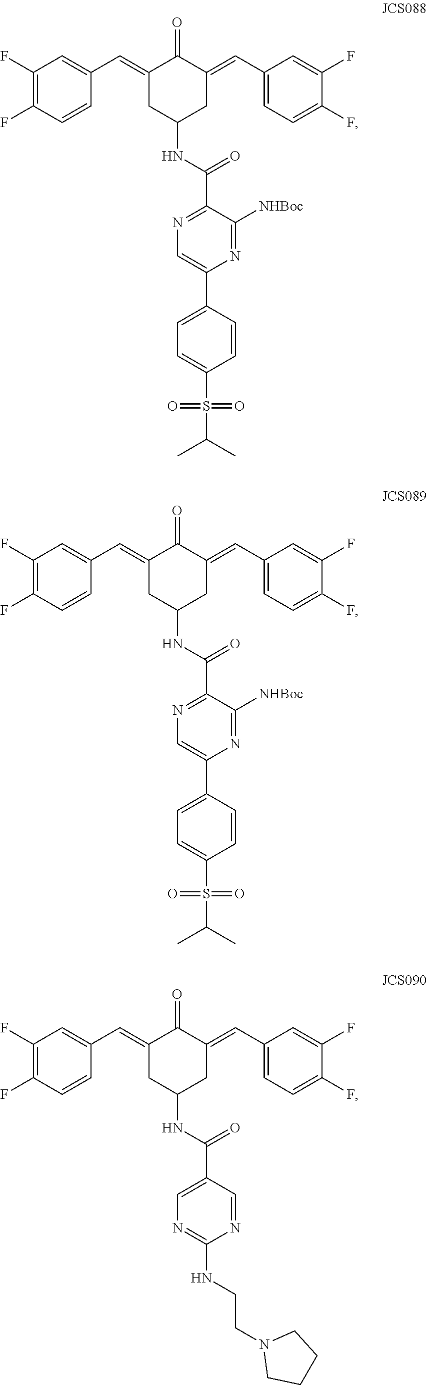 [SMILES: CC(C)S(=O)(=O)c1ccc(-c2cnc(C(=O)NC3C/C(=C\c4ccc(F)c(F)c4)C(=O)/C(=C/c4ccc(F)c(F)c4)C3)c(NC(=O)OC(C)(C)C)n2)cc1 ; CC(C)S(=O)(=O)c1ccc(-c2cnc(C(=O)NC3C/C(=C\c4ccc(F)c(F)c4)C(=O)/C(=C/c4ccc(F)c(F)c4)C3)c(NC(=O)OC(C)(C)C)n2)cc1 ; O=C1/C(=C/c2ccc(F)c(F)c2)CC(NC(=O)c2cnc(NCCN3CCCC3)nc2)C/C1=C\c1ccc(F)c(F)c1]

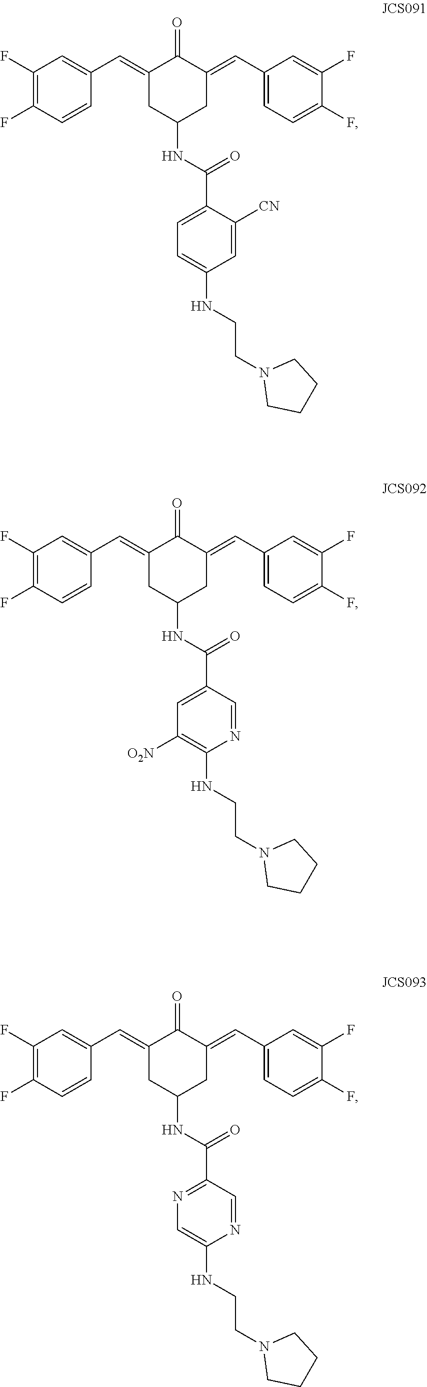 [SMILES: N#Cc1cc(NCCN2CCCC2)ccc1C(=O)NC1C/C(=C\c2ccc(F)c(F)c2)C(=O)/C(=C/c2ccc(F)c(F)c2)C1 ; O=C1/C(=C/c2ccc(F)c(F)c2)CC(NC(=O)c2cnc(NCCN3CCCC3)c([N+](=O)[O-])c2)C/C1=C\c1ccc(F)c(F)c1 ; O=C1/C(=C/c2ccc(F)c(F)c2)CC(NC(=O)c2cnc(NCCN3CCCC3)cn2)C/C1=C\c1ccc(F)c(F)c1]

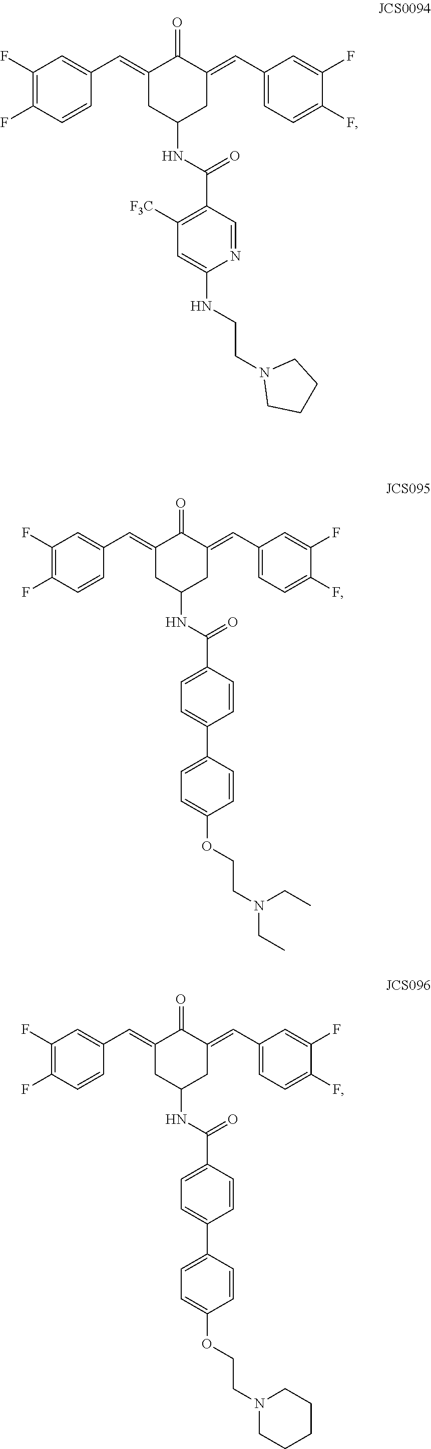 [SMILES: O=C1/C(=C/c2ccc(F)c(F)c2)CC(NC(=O)c2cnc(NCCN3CCCC3)cc2C(F)(F)F)C/C1=C\c1ccc(F)c(F)c1 ; CCN(CC)CCOc1ccc(-c2ccc(C(=O)NC3C/C(=C\c4ccc(F)c(F)c4)C(=O)/C(=C/c4ccc(F)c(F)c4)C3)cc2)cc1 ; O=C1/C(=C/c2ccc(F)c(F)c2)CC(NC(=O)c2ccc(-c3ccc(OCCN4CCCCC4)cc3)cc2)C/C1=C\c1ccc(F)c(F)c1]

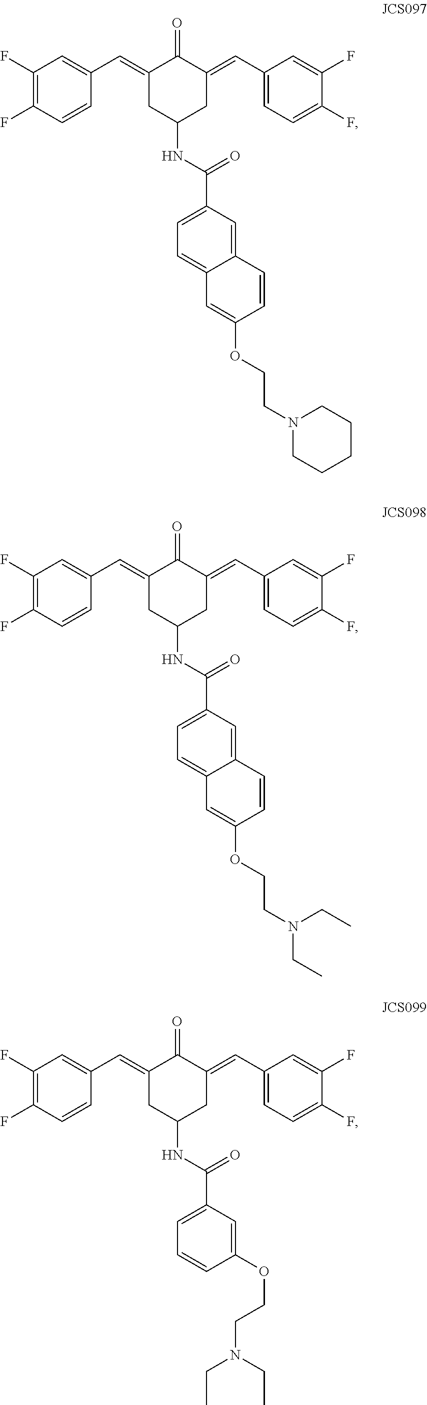 [SMILES: O=C1/C(=C/c2ccc(F)c(F)c2)CC(NC(=O)c2ccc3cc(OCCN4CCCCC4)ccc3c2)C/C1=C\c1ccc(F)c(F)c1 ; CCN(CC)CCOc1ccc2cc(C(=O)NC3C/C(=C\c4ccc(F)c(F)c4)C(=O)/C(=C/c4ccc(F)c(F)c4)C3)ccc2c1 ; CCN(CC)CCOc1cccc(C(=O)NC2C/C(=C\c3ccc(F)c(F)c3)C(=O)/C(=C/c3ccc(F)c(F)c3)C2)c1]

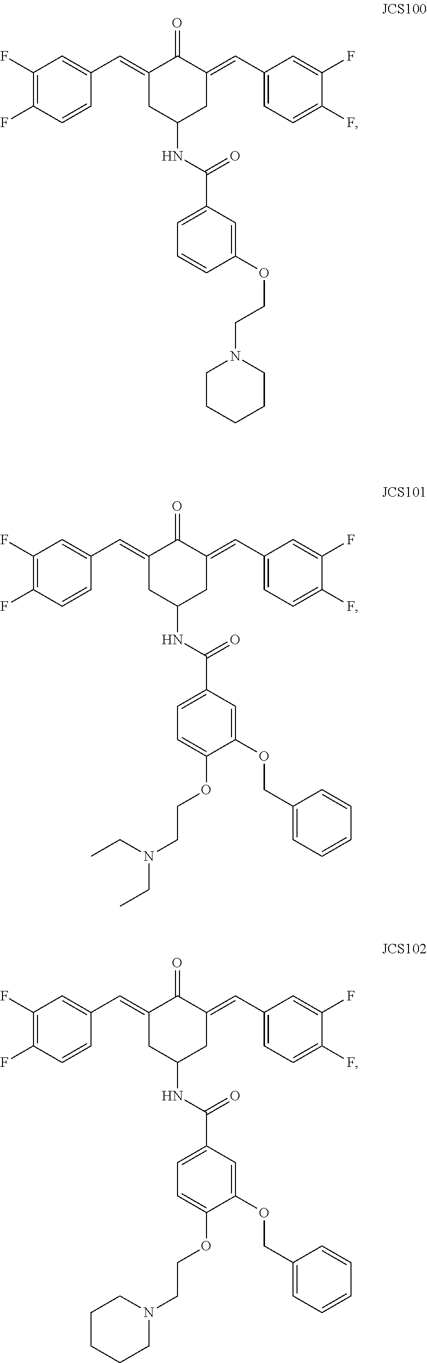 [SMILES: O=C1/C(=C/c2ccc(F)c(F)c2)CC(NC(=O)c2cccc(OCCN3CCCCC3)c2)C/C1=C\c1ccc(F)c(F)c1 ; CCN(CC)CCOc1ccc(C(=O)NC2C/C(=C\c3ccc(F)c(F)c3)C(=O)/C(=C/c3ccc(F)c(F)c3)C2)cc1OCc1ccccc1 ; O=C1/C(=C/c2ccc(F)c(F)c2)CC(NC(=O)c2ccc(OCCN3CCCCC3)c(OCc3ccccc3)c2)C/C1=C\c1ccc(F)c(F)c1]

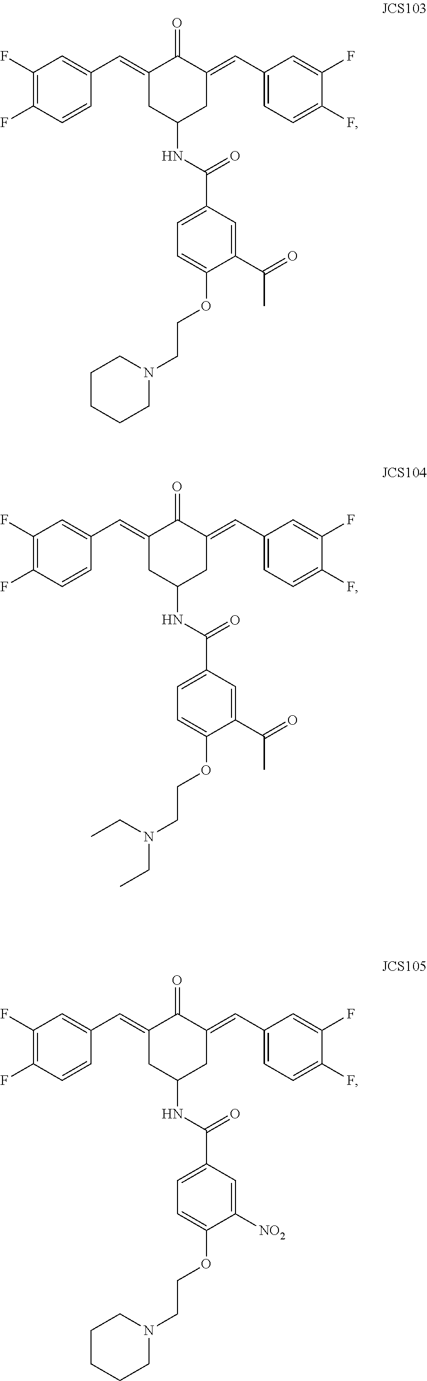 [SMILES: CC(=O)c1cc(C(=O)NC2C/C(=C\c3ccc(F)c(F)c3)C(=O)/C(=C/c3ccc(F)c(F)c3)C2)ccc1OCCN1CCCCC1 ; CCN(CC)CCOc1ccc(C(=O)NC2C/C(=C\c3ccc(F)c(F)c3)C(=O)/C(=C/c3ccc(F)c(F)c3)C2)cc1C(C)=O ; O=C1/C(=C/c2ccc(F)c(F)c2)CC(NC(=O)c2ccc(OCCN3CCCCC3)c([N+](=O)[O-])c2)C/C1=C\c1ccc(F)c(F)c1]

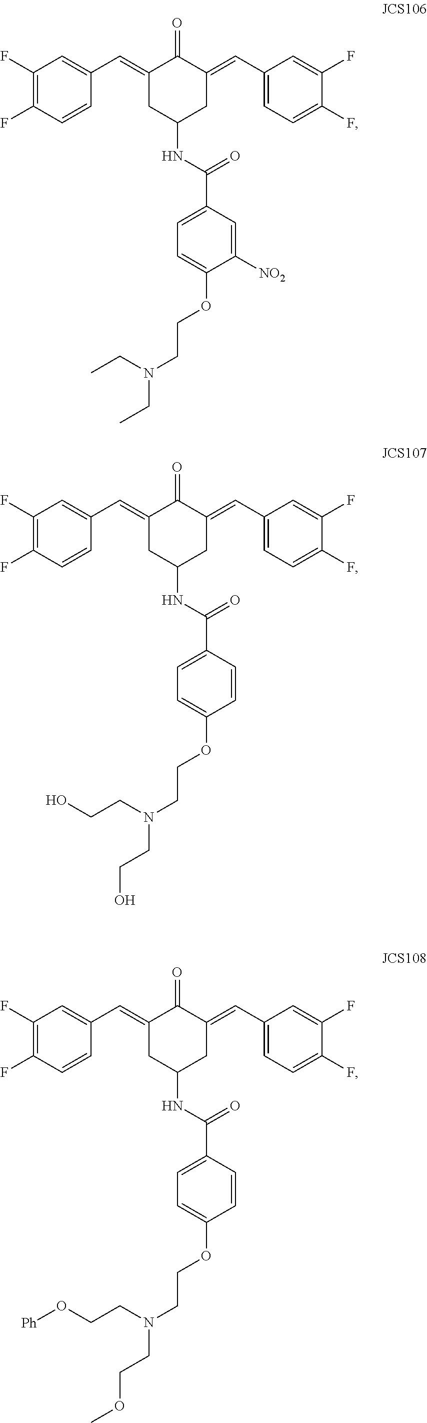 [SMILES: CCN(CC)CCOc1ccc(C(=O)NC2C/C(=C\c3ccc(F)c(F)c3)C(=O)/C(=C/c3ccc(F)c(F)c3)C2)cc1[N+](=O)[O-] ; O=C1/C(=C/c2ccc(F)c(F)c2)CC(NC(=O)c2ccc(OCCN(CCO)CCO)cc2)C/C1=C\c1ccc(F)c(F)c1 ; COCCN(CCOc1ccccc1)CCOc1ccc(C(=O)NC2C/C(=C\c3ccc(F)c(F)c3)C(=O)/C(=C/c3ccc(F)c(F)c3)C2)cc1]

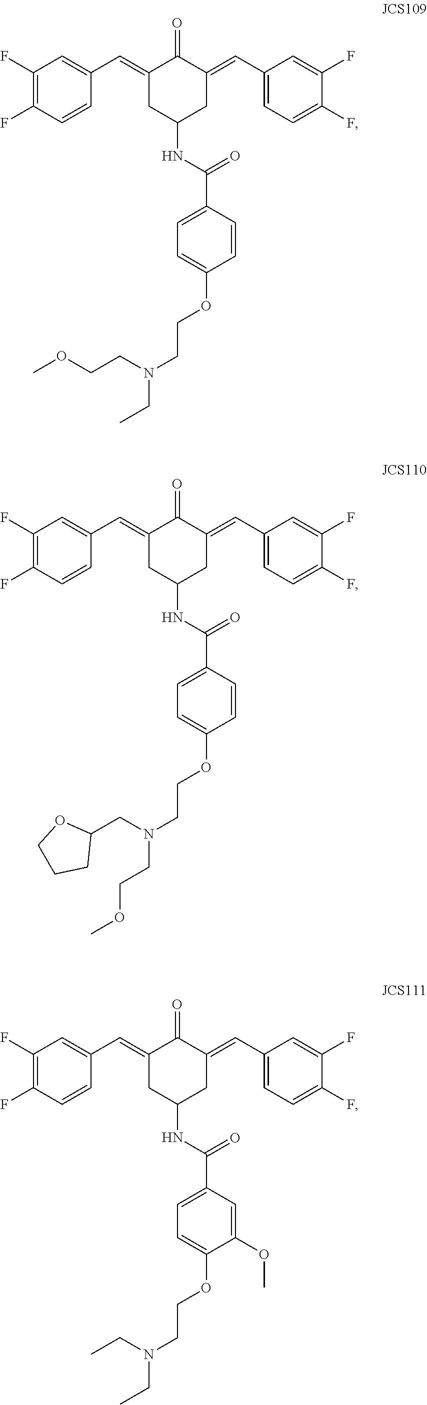 [SMILES: CCN(CCOC)CCOc1ccc(C(=O)NC2C/C(=C\c3ccc(F)c(F)c3)C(=O)/C(=C/c3ccc(F)c(F)c3)C2)cc1 ; COCCN(CCOc1ccc(C(=O)NC2C/C(=C\c3ccc(F)c(F)c3)C(=O)/C(=C/c3ccc(F)c(F)c3)C2)cc1)CC1CCCO1 ; CCN(CC)CCOc1ccc(C(=O)NC2C/C(=C\c3ccc(F)c(F)c3)C(=O)/C(=C/c3ccc(F)c(F)c3)C2)cc1OC]

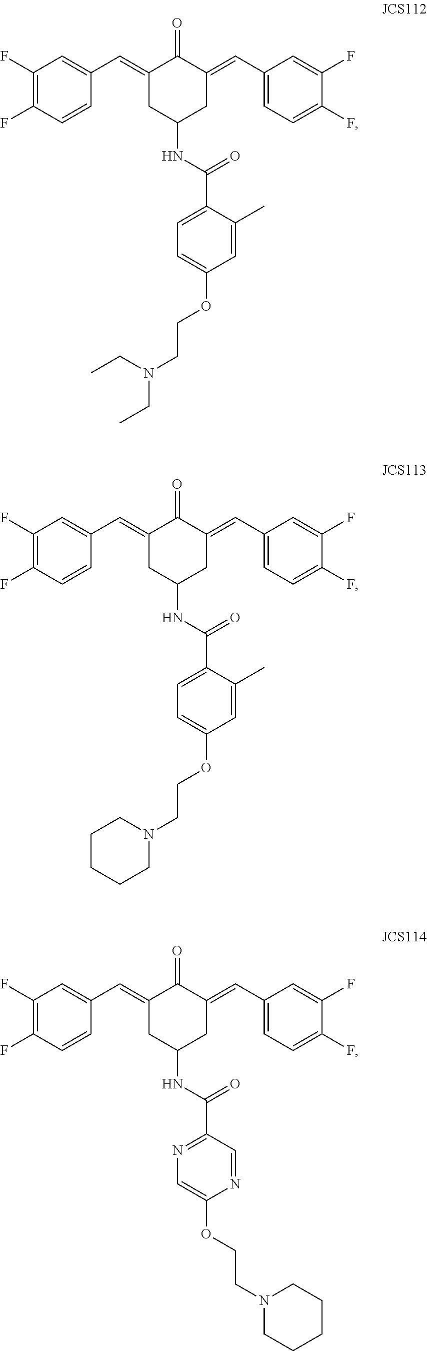 [SMILES: CCN(CC)CCOc1ccc(C(=O)NC2C/C(=C\c3ccc(F)c(F)c3)C(=O)/C(=C/c3ccc(F)c(F)c3)C2)c(C)c1 ; Cc1cc(OCCN2CCCCC2)ccc1C(=O)NC1C/C(=C\c2ccc(F)c(F)c2)C(=O)/C(=C/c2ccc(F)c(F)c2)C1 ; O=C1/C(=C/c2ccc(F)c(F)c2)CC(NC(=O)c2cnc(OCCN3CCCCC3)cn2)C/C1=C\c1ccc(F)c(F)c1]

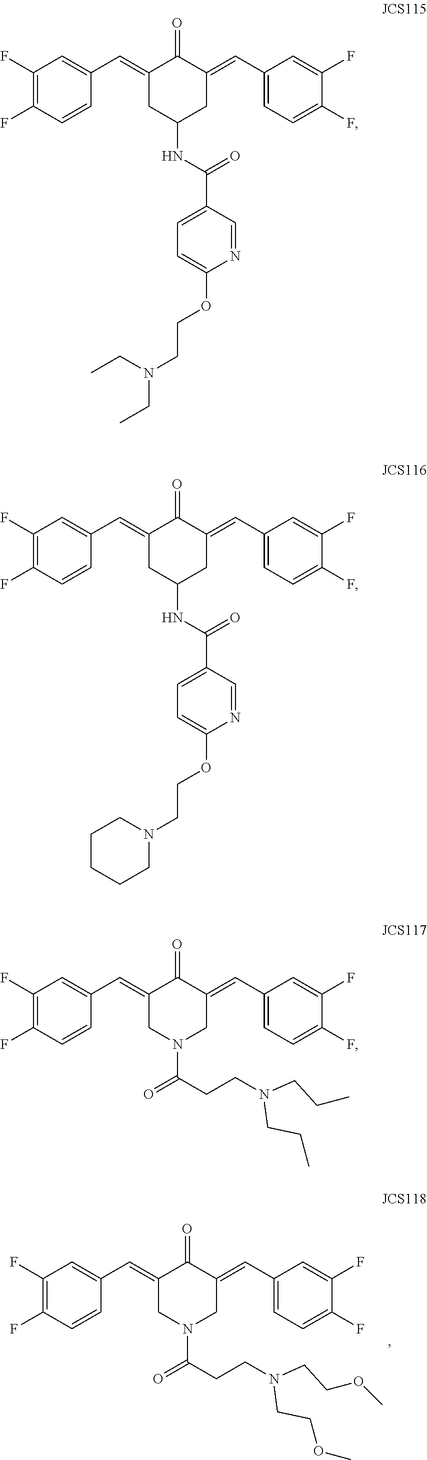 [SMILES: CCN(CC)CCOc1ccc(C(=O)NC2C/C(=C\c3ccc(F)c(F)c3)C(=O)/C(=C/c3ccc(F)c(F)c3)C2)cn1 ; O=C1/C(=C/c2ccc(F)c(F)c2)CC(NC(=O)c2ccc(OCCN3CCCCC3)nc2)C/C1=C\c1ccc(F)c(F)c1 ; CCCN(CCC)CCC(=O)N1C/C(=C\c2ccc(F)c(F)c2)C(=O)/C(=C/c2ccc(F)c(F)c2)C1 ; COCCN(CCOC)CCC(=O)N1C/C(=C\c2ccc(F)c(F)c2)C(=O)/C(=C/c2ccc(F)c(F)c2)C1]

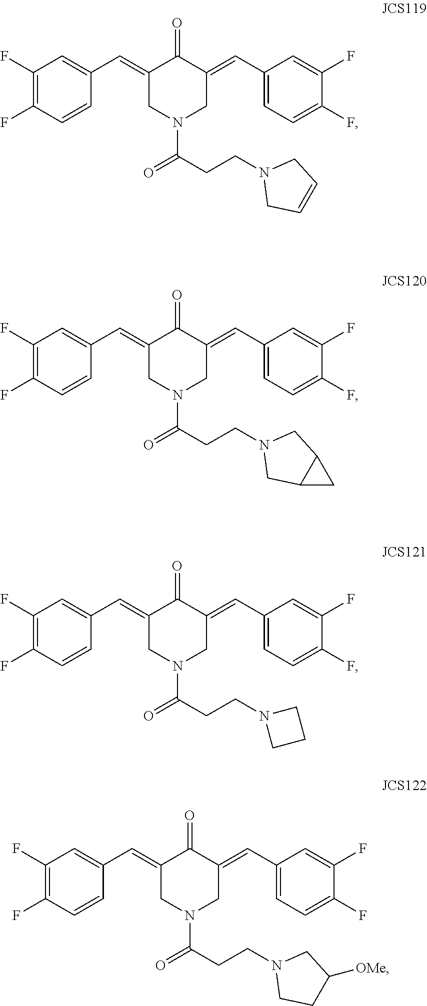 [SMILES: O=C1/C(=C/c2ccc(F)c(F)c2)CN(C(=O)CCN2CC=CC2)C/C1=C\c1ccc(F)c(F)c1 ; O=C1/C(=C/c2ccc(F)c(F)c2)CN(C(=O)CCN2CC3CC3C2)C/C1=C\c1ccc(F)c(F)c1 ; O=C1/C(=C/c2ccc(F)c(F)c2)CN(C(=O)CCN2CCC2)C/C1=C\c1ccc(F)c(F)c1 ; COC1CCN(CCC(=O)N2C/C(=C\c3ccc(F)c(F)c3)C(=O)/C(=C/c3ccc(F)c(F)c3)C2)C1]

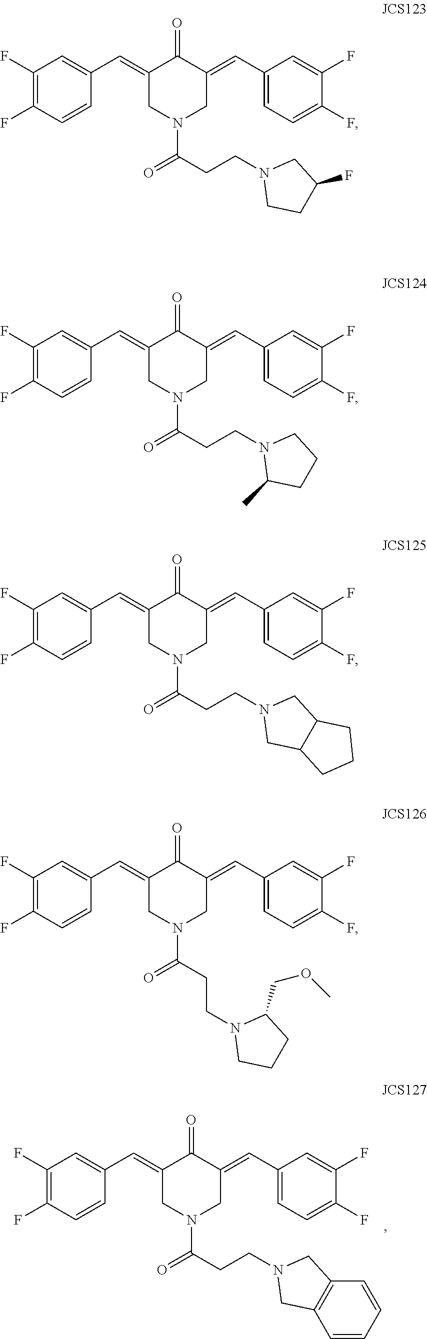 [SMILES: O=C1/C(=C/c2ccc(F)c(F)c2)CN(C(=O)CCN2CC[C@H](F)C2)C/C1=C\c1ccc(F)c(F)c1 ; C[C@@H]1CCCN1CCC(=O)N1C/C(=C\c2ccc(F)c(F)c2)C(=O)/C(=C/c2ccc(F)c(F)c2)C1 ; O=C1/C(=C/c2ccc(F)c(F)c2)CN(C(=O)CCN2CC3CCCC3C2)C/C1=C\c1ccc(F)c(F)c1 ; COC[C@@H]1CCCN1CCC(=O)N1C/C(=C\c2ccc(F)c(F)c2)C(=O)/C(=C/c2ccc(F)c(F)c2)C1 ; O=C1/C(=C/c2ccc(F)c(F)c2)CN(C(=O)CCN2Cc3ccccc3C2)C/C1=C\c1ccc(F)c(F)c1]

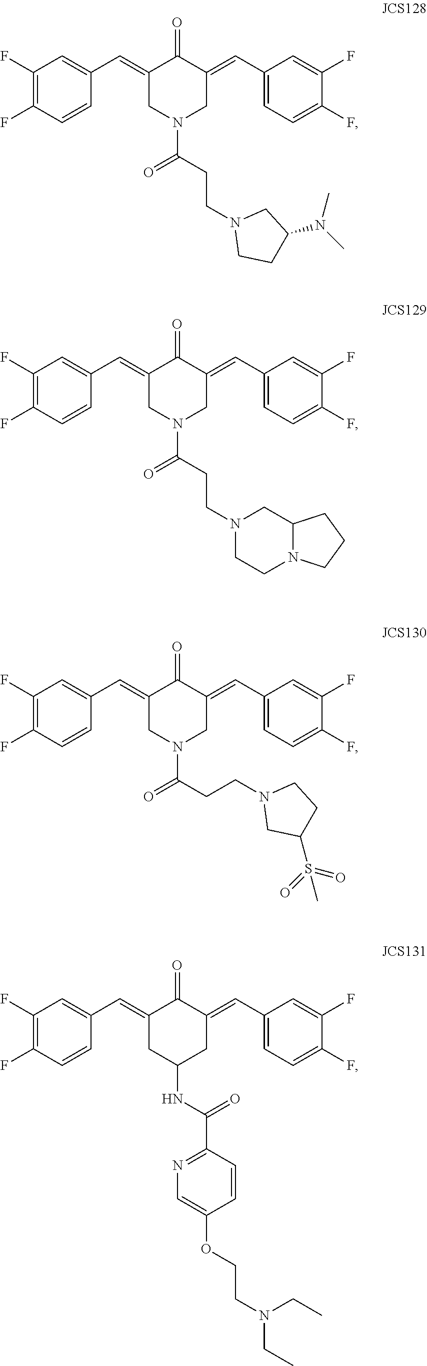 [SMILES: CN(C)[C@@H]1CCN(CCC(=O)N2C/C(=C\c3ccc(F)c(F)c3)C(=O)/C(=C/c3ccc(F)c(F)c3)C2)C1 ; O=C1/C(=C/c2ccc(F)c(F)c2)CN(C(=O)CCN2CCN3CCCC3C2)C/C1=C\c1ccc(F)c(F)c1 ; CS(=O)(=O)C1CCN(CCC(=O)N2C/C(=C\c3ccc(F)c(F)c3)C(=O)/C(=C/c3ccc(F)c(F)c3)C2)C1 ; CCN(CC)CCOc1ccc(C(=O)NC2C/C(=C\c3ccc(F)c(F)c3)C(=O)/C(=C/c3ccc(F)c(F)c3)C2)nc1]

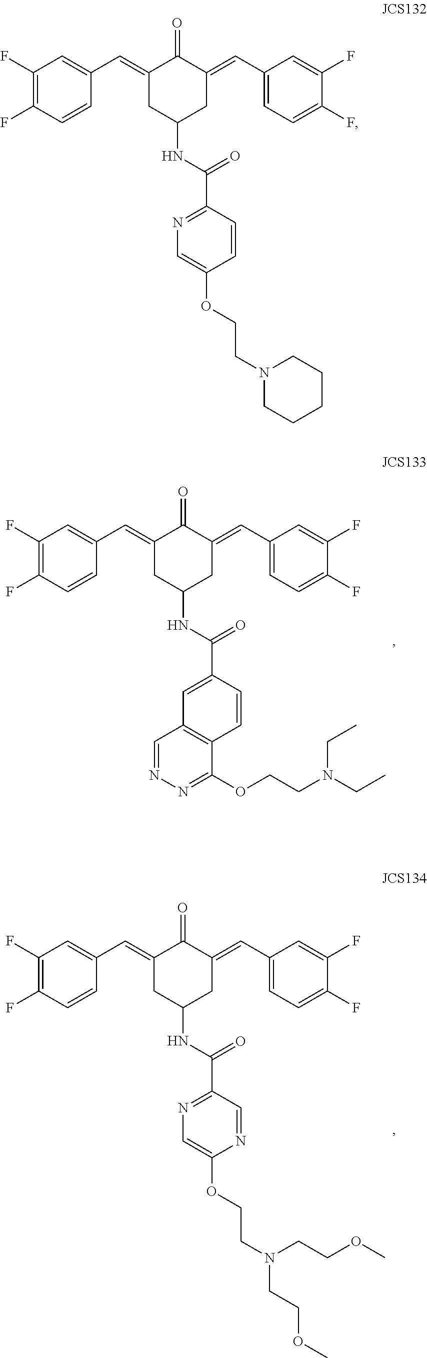 [SMILES: O=C1/C(=C/c2ccc(F)c(F)c2)CC(NC(=O)c2ccc(OCCN3CCCCC3)cn2)C/C1=C\c1ccc(F)c(F)c1 ; CCN(CC)CCOc1nncc2cc(C(=O)NC3C/C(=C\c4ccc(F)c(F)c4)C(=O)/C(=C/c4ccc(F)c(F)c4)C3)ccc12 ; COCCN(CCOC)CCOc1cnc(C(=O)NC2C/C(=C\c3ccc(F)c(F)c3)C(=O)/C(=C/c3ccc(F)c(F)c3)C2)cn1]

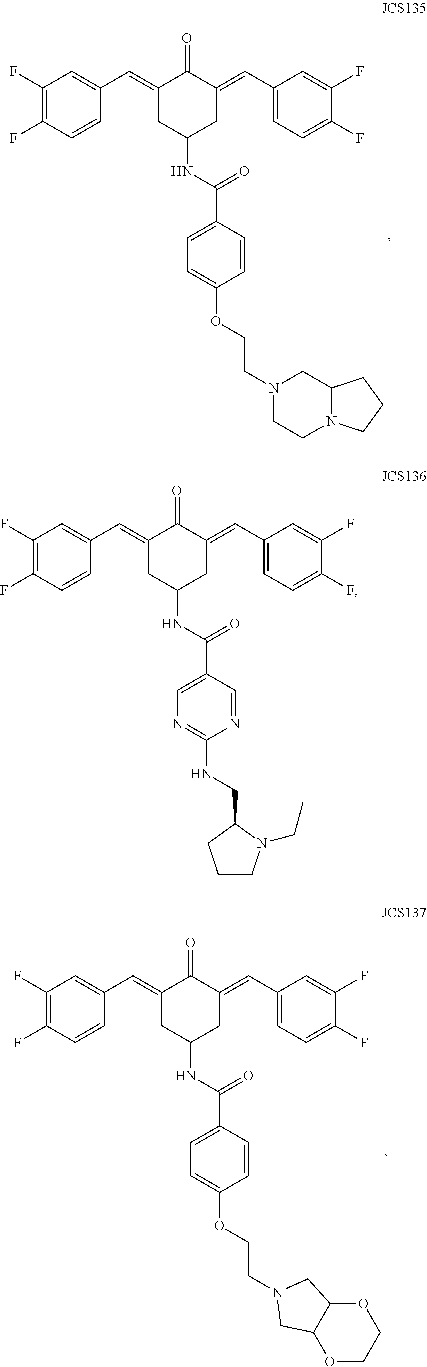 [SMILES: O=C1/C(=C/c2ccc(F)c(F)c2)CC(NC(=O)c2ccc(OCCN3CCN4CCCC4C3)cc2)C/C1=C\c1ccc(F)c(F)c1 ; CCN1CCC[C@H]1CNc1ncc(C(=O)NC2C/C(=C\c3ccc(F)c(F)c3)C(=O)/C(=C/c3ccc(F)c(F)c3)C2)cn1 ; O=C1/C(=C/c2ccc(F)c(F)c2)CC(NC(=O)c2ccc(OCCN3CC4OCCOC4C3)cc2)C/C1=C\c1ccc(F)c(F)c1]

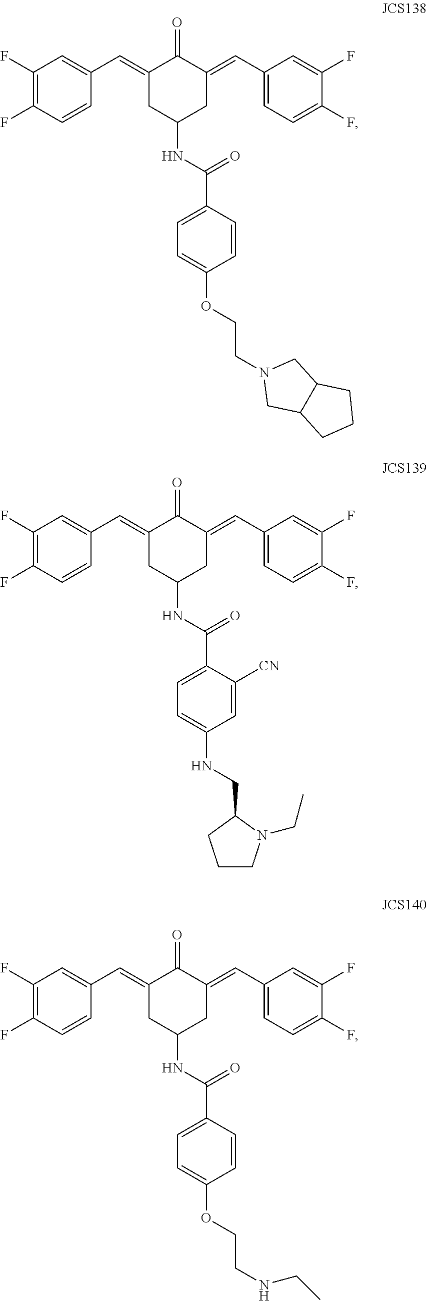 [SMILES: O=C1/C(=C/c2ccc(F)c(F)c2)CC(NC(=O)c2ccc(OCCN3CC4CCCC4C3)cc2)C/C1=C\c1ccc(F)c(F)c1 ; CCN1CCC[C@H]1CNc1ccc(C(=O)NC2C/C(=C\c3ccc(F)c(F)c3)C(=O)/C(=C/c3ccc(F)c(F)c3)C2)c(C#N)c1 ; CCNCCOc1ccc(C(=O)NC2C/C(=C\c3ccc(F)c(F)c3)C(=O)/C(=C/c3ccc(F)c(F)c3)C2)cc1]

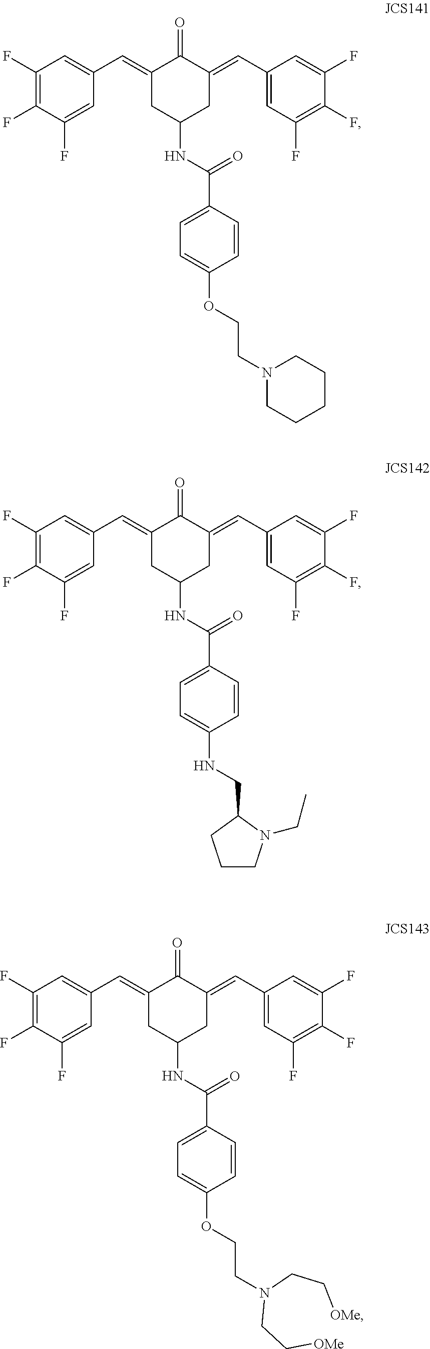 [SMILES: O=C1/C(=C/c2cc(F)c(F)c(F)c2)CC(NC(=O)c2ccc(OCCN3CCCCC3)cc2)C/C1=C\c1cc(F)c(F)c(F)c1 ; CCN1CCC[C@H]1CNc1ccc(C(=O)NC2C/C(=C\c3cc(F)c(F)c(F)c3)C(=O)/C(=C/c3cc(F)c(F)c(F)c3)C2)cc1 ; COCCN(CCOC)CCOc1ccc(C(=O)NC2C/C(=C\c3cc(F)c(F)c(F)c3)C(=O)/C(=C/c3cc(F)c(F)c(F)c3)C2)cc1]

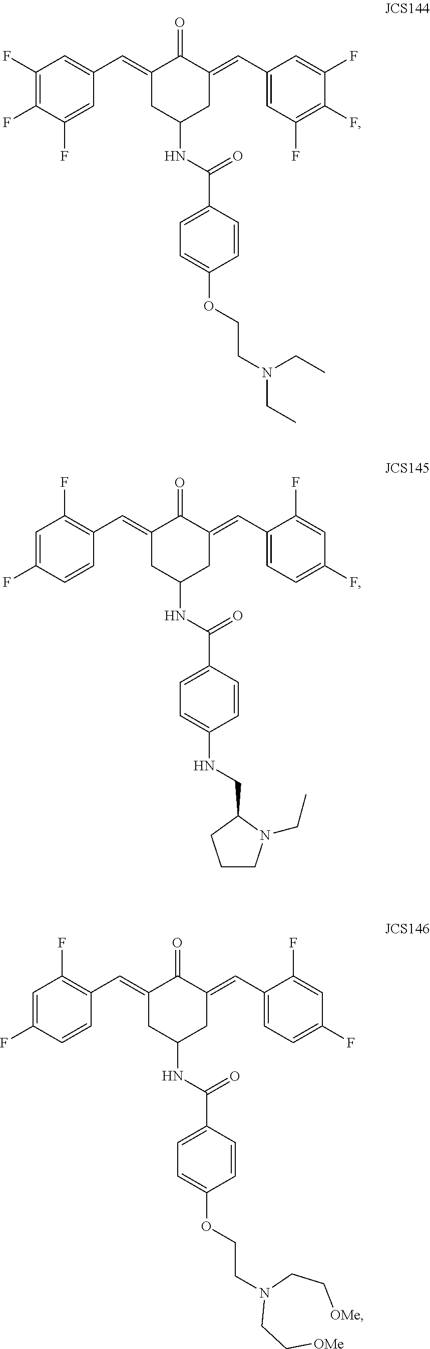 [SMILES: CCN(CC)CCOc1ccc(C(=O)NC2C/C(=C\c3cc(F)c(F)c(F)c3)C(=O)/C(=C/c3cc(F)c(F)c(F)c3)C2)cc1 ; CCN1CCC[C@H]1CNc1ccc(C(=O)NC2C/C(=C\c3ccc(F)cc3F)C(=O)/C(=C/c3ccc(F)cc3F)C2)cc1 ; COCCN(CCOC)CCOc1ccc(C(=O)NC2C/C(=C\c3ccc(F)cc3F)C(=O)/C(=C/c3ccc(F)cc3F)C2)cc1]

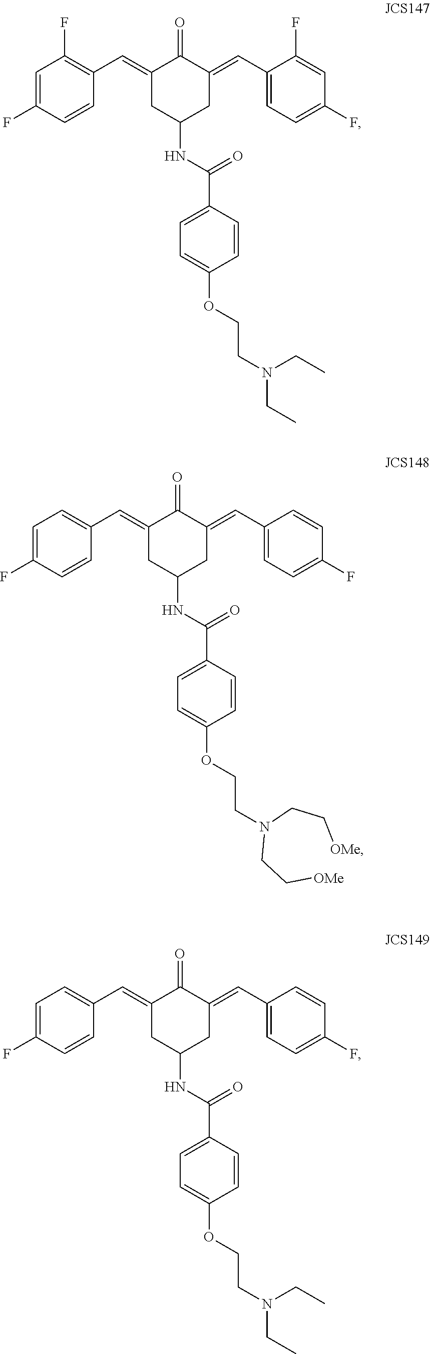 [SMILES: CCN(CC)CCOc1ccc(C(=O)NC2C/C(=C\c3ccc(F)cc3F)C(=O)/C(=C/c3ccc(F)cc3F)C2)cc1 ; COCCN(CCOC)CCOc1ccc(C(=O)NC2C/C(=C\c3ccc(F)cc3)C(=O)/C(=C/c3ccc(F)cc3)C2)cc1 ; CCN(CC)CCOc1ccc(C(=O)NC2C/C(=C\c3ccc(F)cc3)C(=O)/C(=C/c3ccc(F)cc3)C2)cc1]

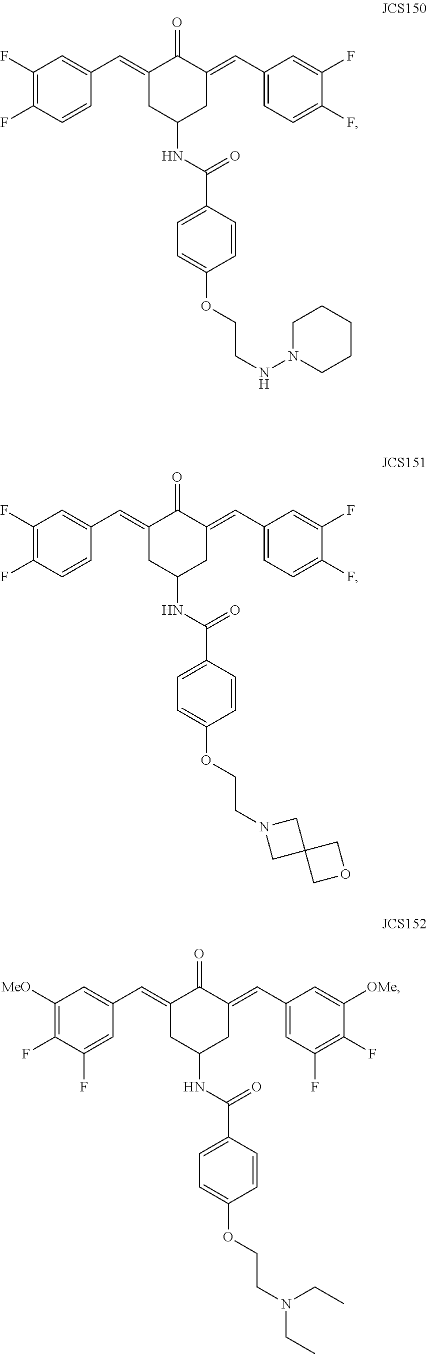 [SMILES: O=C1/C(=C/c2ccc(F)c(F)c2)CC(NC(=O)c2ccc(OCCNN3CCCCC3)cc2)C/C1=C\c1ccc(F)c(F)c1 ; O=C1/C(=C/c2ccc(F)c(F)c2)CC(NC(=O)c2ccc(OCCN3CC4(COC4)C3)cc2)C/C1=C\c1ccc(F)c(F)c1 ; CCN(CC)CCOc1ccc(C(=O)NC2C/C(=C\c3cc(F)c(F)c(OC)c3)C(=O)/C(=C/c3cc(F)c(F)c(OC)c3)C2)cc1]

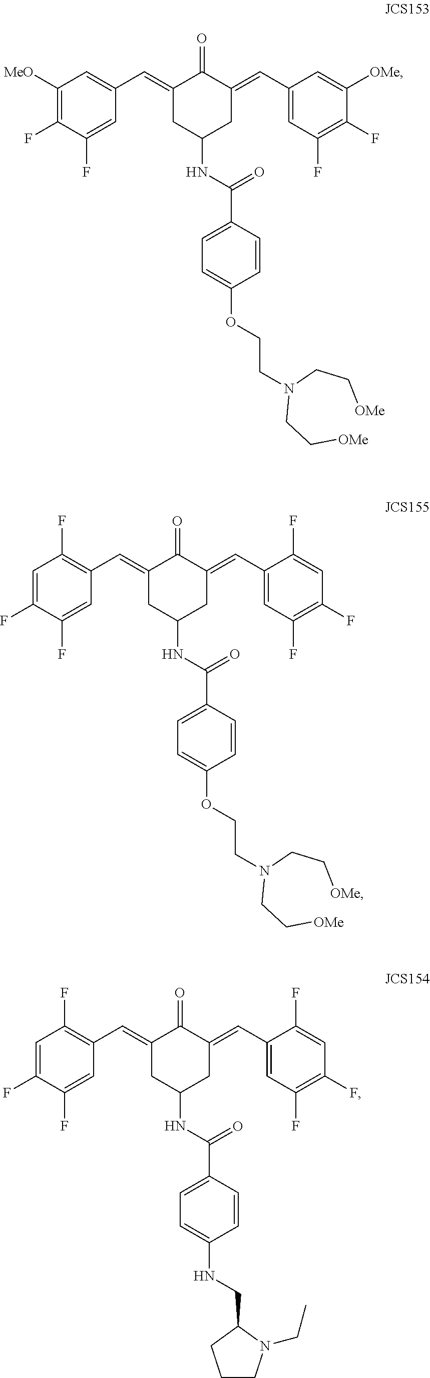 [SMILES: COCCN(CCOC)CCOc1ccc(C(=O)NC2C/C(=C\c3cc(F)c(F)c(OC)c3)C(=O)/C(=C/c3cc(F)c(F)c(OC)c3)C2)cc1 ; COCCN(CCOC)CCOc1ccc(C(=O)NC2C/C(=C\c3cc(F)c(F)cc3F)C(=O)/C(=C/c3cc(F)c(F)cc3F)C2)cc1 ; CCN1CCC[C@H]1CNc1ccc(C(=O)NC2C/C(=C\c3cc(F)c(F)cc3F)C(=O)/C(=C/c3cc(F)c(F)cc3F)C2)cc1]

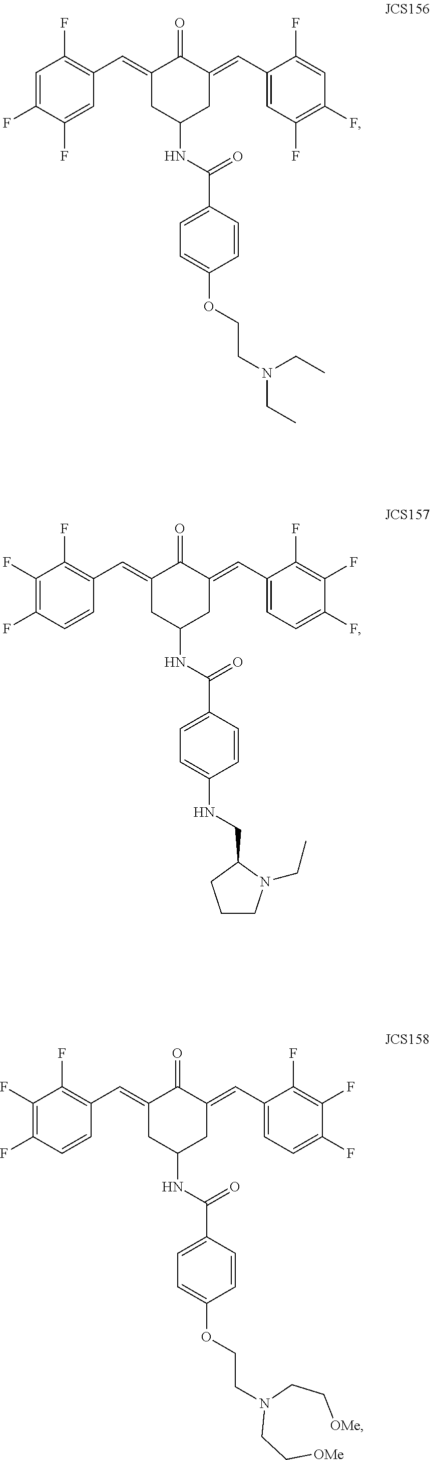 [SMILES: CCN(CC)CCOc1ccc(C(=O)NC2C/C(=C\c3cc(F)c(F)cc3F)C(=O)/C(=C/c3cc(F)c(F)cc3F)C2)cc1 ; CCN1CCC[C@H]1CNc1ccc(C(=O)NC2C/C(=C\c3ccc(F)c(F)c3F)C(=O)/C(=C/c3ccc(F)c(F)c3F)C2)cc1 ; COCCN(CCOC)CCOc1ccc(C(=O)NC2C/C(=C\c3ccc(F)c(F)c3F)C(=O)/C(=C/c3ccc(F)c(F)c3F)C2)cc1]

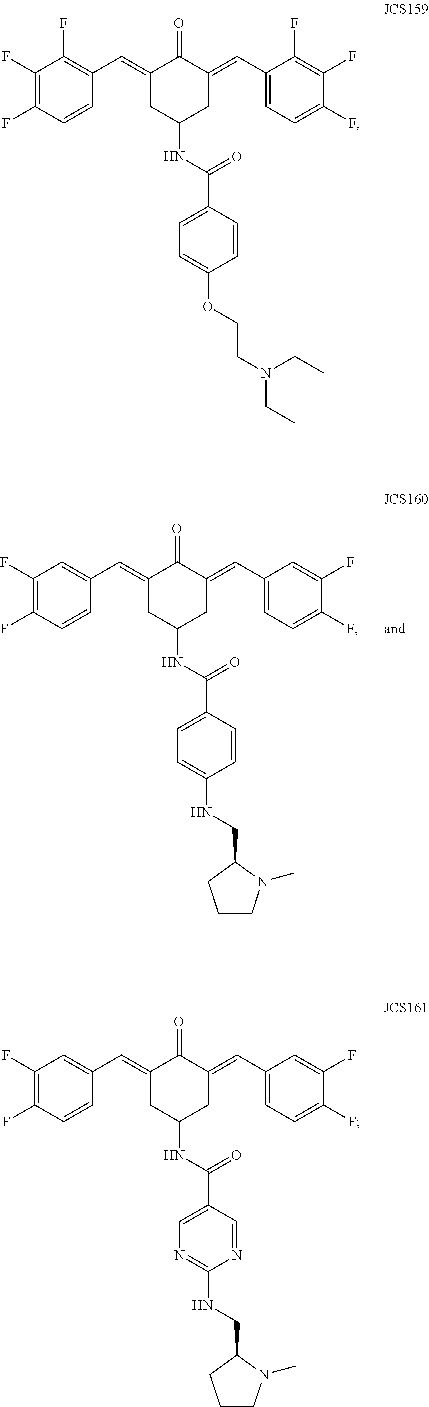 [SMILES: CCN(CC)CCOc1ccc(C(=O)NC2C/C(=C\c3ccc(F)c(F)c3F)C(=O)/C(=C/c3ccc(F)c(F)c3F)C2)cc1 ; CN1CCC[C@H]1CNc1ccc(C(=O)NC2C/C(=C\c3ccc(F)c(F)c3)C(=O)/C(=C/c3ccc(F)c(F)c3)C2)cc1 ; CN1CCC[C@H]1CNc1ncc(C(=O)NC2C/C(=C\c3ccc(F)c(F)c3)C(=O)/C(=C/c3ccc(F)c(F)c3)C2)cn1]

or a pharmaceutically acceptable salt thereof.

In another aspect, the present disclosure provides a pharmaceutical composition, comprising a compound disclosed herein and a pharmaceutically acceptable excipient.

In yet another aspect, the present disclosure provides methods of treating cancer in a subject in need thereof, comprising administering to the subject a compound disclosed herein or a pharmaceutically acceptable salt thereof.

In certain embodiments, the cancer is breast cancer, head and neck cancer, lung cancer, prostate cancer, or ovarian cancer. In further embodiments, the cancer is testicular cancer, cervical cancer, bladder cancer, esophageal cancer, mesothelioma, or brain cancer (e.g., neuroblastoma).

In certain embodiments, the cancer is relapsed. In certain embodiments, the cancer is refractory.

In certain embodiments, the cancer is resistant to treatment with olaparib. In certain embodiments, the cancer is resistant to treatment with cisplatin.

In yet another aspect, the present disclosure provides methods of inhibiting repair of DNA in a subject in need thereof, comprising administering to the subject a compound disclosed herein or a pharmaceutically acceptable salt thereof.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto.

Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-30 for straight chains, C₃-30 for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁-6alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amido”, as used herein, refers to a group

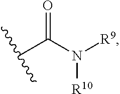

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

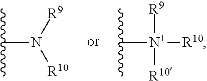

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

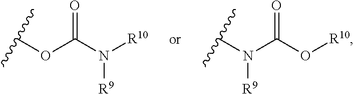

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamido” is art-recognized and refers to the group represented by the general formulae

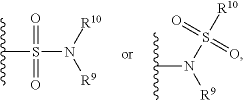

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

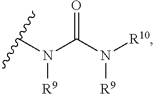

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### EXAMPLES

The invention now being generally described, it will be more readily understood by reference to the following examples which are included merely for purposes of illustration of certain aspects and embodiments of the present invention and are not intended to limit the invention.

### Example 1: Synthesis of Exemplary Compounds of the Disclosure

### General Procedure A:

The compounds were generally prepared by reaction of the corresponding aldehydes, e.g., 3,4-difluorobenzaldehyde, with tert-butyl (4-oxocyclohexyl)carbamate in the present of 20% aq. sodium hydroxide to give tert-butyl (3,5-bis((E)-3,4-difluorobenzylidene)-4-oxocyclohexyl)carb-amate. TFA deprotected Boc group. Benzoylation of 4-amino-2,6-bis((E)-3,4-difluorobenzylidene)cyclo-hexan-1-one with 4-(2-(piperidin-1-yl)ethoxy)benzoyl chloride under base condition afforded the N-(3,5-Bis((E)-3,4-difluorobenzylidene)-4-oxocyclohexyl)-4-(2-(piperidin-1-yl)ethoxy)benzamide.

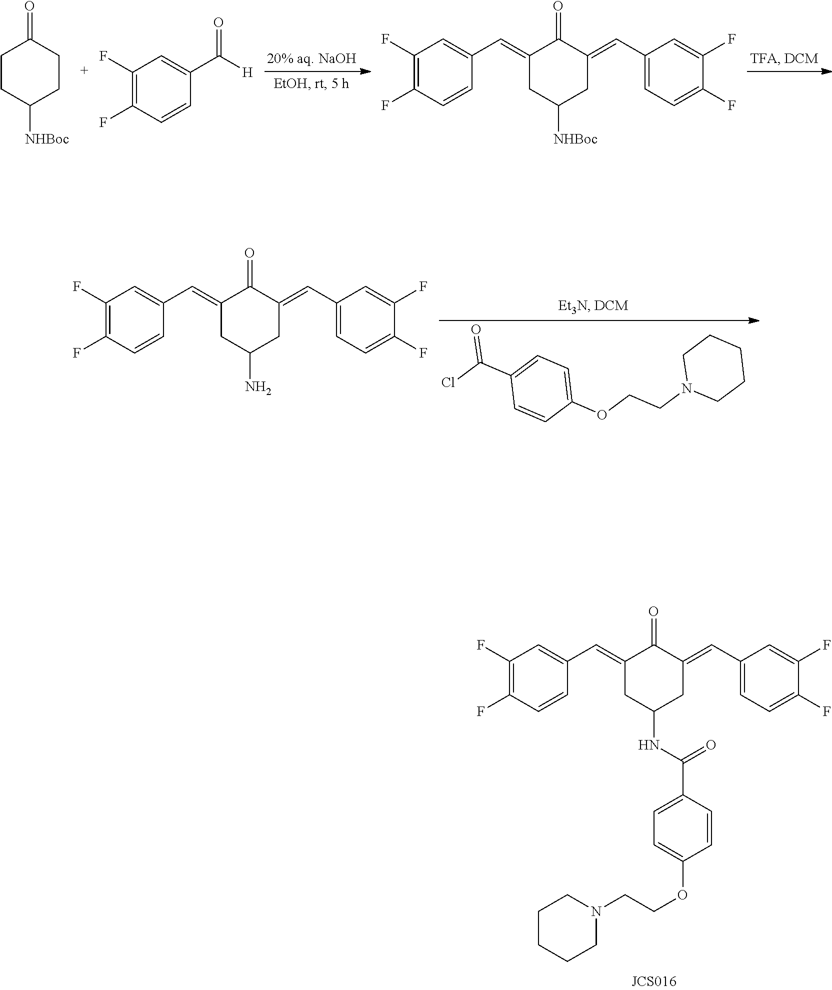

### Example JCS016

To the mixture of the tert-butyl (4-oxocyclohexyl)carbamate (213.28 mg, 1 mmol, 1.0 equiv.) and ethanol (1.0 mL) in a round bottom flask added drop-wise 20% aqueous sodium hydroxide (1.5 mL) and stirred for five minutes. To this mixture was added 3,4-difluorobenzaldehyde (355.3 mg, 2.5 mmol, 2.5 equiv.). The reaction mixture was then allowed to stir at room temperature for 5 h. After 5 h the yellow precipitate thus obtained was filtered, washed with water, cold ethanol and dried to get pure product (360 mg, 78% yield).

Trifluoroacetic acid (0.5 ml) was added to a solution of tert-butyl (3,5-bis((E)-3,4-difluorobenzylidene)-4-oxocyclohexyl)carbamate (230.7 mg, 0.5 mmol) in methylene chloride (5.0 ml) at room temperature and stirred overnight at room temperature. Then, the solvent of the reaction solution was distilled off under reduced pressure and the resulting residue was poured into a 1N-aqueous sodium hydroxide solution and extracted with ethyl acetate. The organic layer was washed with a saturated aqueous sodium chloride solution and then dried over anhydrous magnesium sulfate. The solvent was distilled off under reduced pressure to obtain 4-amino-2,6-bis((E)-3,4-difluorobenzylidene)cyclohexan-1-one.

The mixture of 4-amino-2,6-bis((E)-3,4-difluorobenzylidene)cyclohexan-1-one (180.7 mg, 0.5 mmol, 1.0 equiv.) and anhydrous triethylamine (70 μL, 0.5 mmol, 1.0 equiv.) in dichloromethane was maintained at 0° C. (ice bath). To this cooled mixture, 4-(2-(piperidin-1-yl)ethoxy)benzoyl chloride (133.8 mg, 0.5 mmol, 1.0 equiv.) in 2.0 mL dichloromethane was added drop wise. After the complete addition of 4-(2-(dimethylamino)ethoxy)benzoyl chloride the reaction mixture was slowly warmed up to room temperature and stirred overnight. After completion of the reaction solvent was evaporated and the residue was stirred in sat aqueous K₂CO₃ for 4 h. The mixture was extracted with ethyl acetate three times. The organic layer was washed with a saturated aqueous sodium chloride solution and then dried over anhydrous sodium sulfate. The solvent was evaporated, followed by flash chromatography (gradient elution 10% methanol/EtOAc-50% methanol/EtOAc) to give yellow solid. The compound was recrystallized with pure diethyl ether in freezer overnight to get pure compound JCS016 (222.2 mg, 75% yield).

### Synthesis of 4-(2-(piperidin-1-yl)ethoxy)benzoyl chloride Method 1

Methyl 4-hydroxybenzoate (5.0 g, 32.86 mmol, 10. equiv) was combined with 1,2-dibromoethane (35 mL) and potassium carbonate (6.8 g, 49.23 mmol, 1.5 equiv) then the mixture was heated at reflux for 18 h. The reaction mixture was concentrated under reduced pressure and then the residue was partitioned between ethyl ether (300 mL) and water (200 mL). The ether layer was extracted with 2 N sodium hydroxide (5×30 mL). The solvent was removed to give the desired product as a white solid (8.5 g, 99% yield).

To a round bottom flask equipped with a stir-bar was added a solution of methyl 4-(2-bromoethoxy)-benzoate (2.591 g, 10 mmol, 1.0 equiv), DMF (20 mL), potassium carbonate (4.146 g, 30 mmol, 3.0 equiv) and piperidine (2.6 g, 30 mmol, 3.0 equiv). The mixture was heated at 75° C. for 24 h, after which time EtOAc (200 mL) and water (200 mL) were added. The organic layer was washed with water three times and then dried over sodium sulfate. The pure product was obtained by flash chromatography on silica gel (gradient elution 10% methanol/EtOAc-50% methanol/EtOAc) to give the desired product, methyl 4-(2-(diethylamino)ethoxy)benzoate (1.97 g, 75% yield), as a light yellow oil.

Methyl 4-(2-(piperidin-1-yl)ethoxy)benzoate (1.31 g, 5.0 mmol, 1.0 equiv) was dissolved in 2.5 mL ethanol and added to a solution of sodium hydroxide (0.4 g) in 2.5 mL water. The mixture was heated under reflux for 2 h. The ethanol was removed in vacuo and the aqueous solution was acidified with cone. HCl at 5° C. The solid was collected, treated with cold water, filtered and dried at 55-60° C. in vacuo to give 4-(2-(piperidin-1-yl)ethoxy)benzoic acid hydrochloride as white sold (1.28 g, 90% yield).

To a stirred mixture of 4-(2-(piperidin-1-yl)ethoxy)benzoic acid hydrochloride (285.11 mg, 1.0 mmol) was added thionyl chloride (2.5 mL). The mixture was heated at reflux for 4 h. The thionyl chloride was removed in vacuo and the residue dried to give 4-(2-(piperidin-1-yl)ethoxy)benzoyl chloride which was enough pure to be used for next step.

The following compounds were synthesized by procedure A: JCS008, JCS0009, JCS010, JCS11, JCS12, JCS13, JCS14, JCS015, JCS016, JCS017.

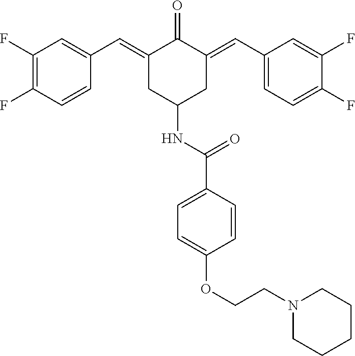 [SMILES: O=C1/C(=C/c2ccc(F)c(F)c2)CC(NC(=O)c2ccc(OCCN3CCCCC3)cc2)C/C1=C\c1ccc(F)c(F)c1]

### N-(3,5-Bis((E)-3,4-difluorobenzylidene)-4-oxocyclohexyl)-4-(2-(piperidin-1-yl)ethoxy)benzamide (JCS016)

¹H NMR (500 MHz, CDCl₃) δ 7.76 (s, 2H), 7.63 (d, J=8.8 Hz, 2H), 7.31-7.09 (m, 6H), 6.86 (d, J=8.8 Hz, 2H), 6.24 (d, J=7.2 Hz, 1H), 4.50-4.44 (m, 1H), 4.11 (t, J=6.0 Hz, 2H), 3.24 (bd, J=15.8 Hz, 2H), 3.04 (dd, J=15.8, 8.3 Hz, 2H), 2.76 (t, J=6.0 Hz, 2H), 2.59-2.37 (m, 4H), 1.60 (p, J=5.6 Hz, 4H), 1.43 (m, 2H). ¹³C NMR (126 MHz, CDCl₃) δ 187.9, 166.6, 161.7, 150.7 (dd, J_{CF}=254.5 Hz, J_{C-CF}=13.8 Hz, 2C), 150.3 (dd, J_{CF}=249.4 Hz, J_{C-CF}=12.6 Hz, 2C), 137.8 (2C), 132.8 (2C), 132.2 (dd, J_{C-C-CF}=6.3 Hz, J_{C-C-CF}=5.0 Hz, 2C), 128.8 (2C), 127.1 (dd, J_{C-C-CF}=6.3 Hz, J_{C-C-CF}=3.8 Hz, 2C), 126.2, 119.0 (d, J_{C-CF}=17.6 Hz, 2C), 117.7 (d, J_{C-CF}=17.6 Hz, 2C), 114.4 (2C), 66.2, 57.8, 55.1, 44.6, 33.8, 25.9, 24.1. ¹⁹F NMR (376 MHz, CDCl₃) δ −135.0 (d, J_{FF}=18.8 Hz, 2F), −136.5 (d, J_{FF}=22.5 Hz, 2F). HR-APCI m/z calcd for C₃₄H₃₃F₄N₂O₃ [M+H]=593.2421, found 593.2444.

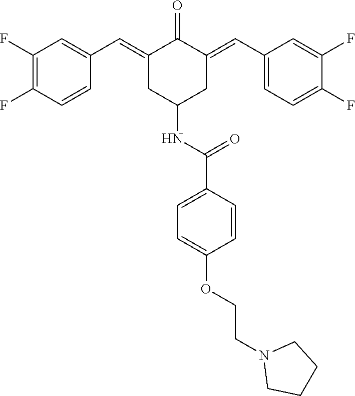 [SMILES: O=C1/C(=C/c2ccc(F)c(F)c2)CC(NC(=O)c2ccc(OCCN3CCCC3)cc2)C/C1=C\c1ccc(F)c(F)c1]

### N-(3,5-Bis((E)-3,4-difluorobenzylidene)-4-oxocyclohexyl)-4-(2-(pyrrolidin-1-yl)ethoxy)benzamide (JCS008)

¹H NMR (500 MHz, CDCl₃) δ 7.73 (s, 2H), 7.63 (d, J=8.7 Hz, 2H), 7.23-7.06 (m, 6H), 6.86 (d, J=8.6 Hz, 2H), 6.34 (d, J=7.2 Hz, 1H), 4.47-4.43 (m, 1H), 4.09 (t, J=5.9 Hz, 2H), 3.22 (bd, J=15.7 Hz, 2H), 3.02 (dd, J=15.7, 8.5 Hz, 2H), 2.87 (t, J=5.9 Hz, 2H), 2.60-2.58 (m, 4H), 1.89-1.69 (m, 4H).

¹³C NMR (126 MHz, CDCl₃) δ 187.8, 166.7, 161.8, 150.6 (dd, J_{CF}=253.2 Hz, J_{C-CF}=12.6 Hz, 2C), 150.2 (dd, J_{CF}=249.4 Hz, J_{C-CF}=13.8 Hz, 2C), 137.7 (2C), 132.8 (2C), 132.2 (dd, J_{C-C-CF}=6.3 Hz, J_{C-C-CF}=3.7 Hz, 2C), 128.8 (2C), 127.1 (dd, J_{C-C-CF}=6.3 Hz, J_{C-C-CF}=3.8 Hz, 2C), 126.1, 118.9 (d, J_{C-CF}=17.6 Hz, 2C), 117.7 (d, J_{C-CF}=17.6 Hz, 2C), 114.4 (2C), 67.3, 54.9, 54.8, 44.6, 33.7, 23.5. ¹⁹F NMR (376 MHz, CDCl₃) δ −135.0 (d, J_{FF}=18.8 Hz, 2F), −136.5 (d, J_{FF}=22.5 Hz, 2F). HR-APCI m/z calcd for C₃₃H₃₁F₄N₂O₃ [M+H]=579.2265, found 579.2318.

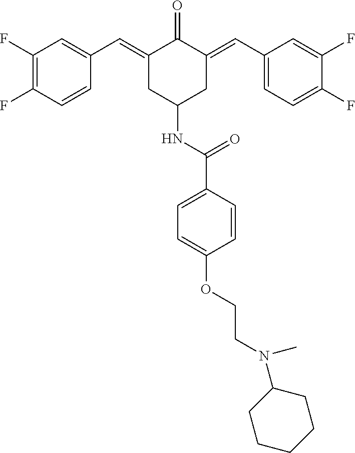 [SMILES: CN(CCOc1ccc(C(=O)NC2C/C(=C\c3ccc(F)c(F)c3)C(=O)/C(=C/c3ccc(F)c(F)c3)C2)cc1)C1CCCCC1]

### N-(3,5-Bis((E)-3,4-difluorobenzylidene)-4-oxocyclohexyl)-4-(2-(cyclohexyl(methyl)amino)ethoxy)-benzamide (JCS009)

¹H NMR (500 MHz, CDCl₃) δ 7.72 (s, 2H), 7.64 (d, J=8.8 Hz, 2H), 7.24-7.09 (m, 6H), 6.83 (d, J=8.8 Hz, 2H), 6.52 (d, J=7.4 Hz, 1H), 4.44-4.40 (m, 1H), 4.07 (t, J=6.0 Hz, 2H), 3.22 (bd, J=15.9, 8.8, Hz, 2H), 3.02 (dd, J=15.9, 8.8, Hz, 2H), 2.90 (t, J=6.0 Hz, 2H), 2.55-2.45 (m, 1H), 2.40 (s, 3H), 1.91-1.73 (m, 4H), 1.67-1.57 (m, 1H), 1.31-1.15 (m, 4H), 1.07 (dtt, J=12.6, 9.0, 3.7 Hz, 1H), 1.11-1.03 (m, 1H). ¹³C NMR (126 MHz, CDCl₃) δ 187.9, 166.7, 161.5, 150.6 (dd, J_{CF}=253.2 Hz, J_{C-CF}=12.6 Hz, 2C), 150.2 (dd, J_{CF}=249.4 Hz, J_{C-CF}=12.6 Hz, 2C), 137.6 (2C), 132.8 (2C), 132.2 (dd, J_{C-C-CF}=6.3 Hz, J_{C-C-CF}=3.7 Hz, 2C), 128.8 (2C), 127.1 (dd, J_{C-C-CF}=6.3 Hz, J_{C-C-CF}=2.5 Hz, 2C), 126.3, 118.9 (d, J_{C-CF}=17.6 Hz, 2C), 117.7 (d, J_{C-CF}=17.6 Hz, 2C), 114.4 (2C), 66.6, 63.7, 52.1, 44.7, 38.7, 3.8, 28.3, 26.1, 25.8. ¹⁹F NMR (376 MHz, CDCl₃) δ −135.1 (d, J_{FF}=22.5 Hz, 2F), −136.5 (d, J_{FF}=22.5 Hz, 2F). HR-APCI m/z calcd for C₃₆H₃₇F₄N₂O₃ [M+H]=621.2760, found 621.2760.

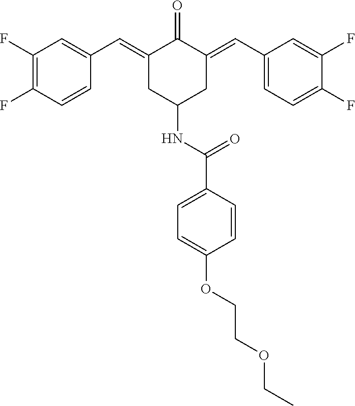 [SMILES: CCOCCOc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-(2-ethoxyethoxy)benzamide (JCSO10)

¹H NMR (500 MHz, CDCl₃) δ 7.81 (s, 2H), 7.62 (d, J=8.9 Hz, 2H), 7.26-7.18 (m, 6H), 6.92 (d, J=8.9 Hz, 2H), 6.04 (d, J=7.3 Hz, 1H), 4.54-4.47 (m, 1H), 4.18-4.10 (m, 2H), 3.83-3.76 (m, 2H), 3.60 (q, J=7.0 Hz, 2H), 3.26 (bd, J=16.7 Hz, 2H), 3.06 (dd, J=16.7, 8.9 Hz, 2H), 1.24 (t, J=7.0 Hz, 3H). ¹³C NMR (126 MHz, CDCl₃) δ 188.0, 166.7, 161.8, 150.8 (dd, J_{CF}=254.5 Hz, J_{C-CF}=12.6 Hz, 2C), 150.3 (dd, J_{CF}=249.4 Hz, J_{C-CF}=12.6 Hz, 2C), 138.0 (2C), 132.8 (2C), 132.2 (dd, J_{C-C-CF}=5.0 Hz, J_{C-C-CF}=3.7 Hz, 2C), 128.7 (2C), 127.2 (dd, J_{C-C-CF}=6.3 Hz, J_{C-C-CF}=3.7 Hz, 2C), 126.38, 119.1 (d, J_{C-CF}=17.6 Hz, 2C), 117.8 (d, J_{C-CF}=17.6 Hz, 2C), 114.4 (2C), 68.8, 67.7, 67.0, 44.6, 33.8, 15.2. ¹⁹F NMR (376 MHz, CDCl₃) δ −135.0 (d, J_{FF}=18.8 Hz, 2F), −136.5 (d, J_{FF}=22.5 Hz, 2F). HR-APCI m/z calcd for C₃₁H₂₈F₄NO₄ [M+H]=554.1973, found 554.1948.

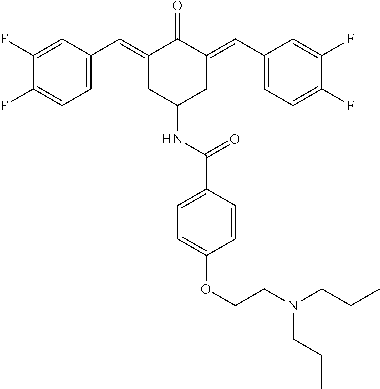 [SMILES: CCCN(CCC)CCOc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-(2-(dipropylamino)ethoxy)benzamide (JCS011)

¹H NMR (500 MHz, CDCl₃) δ 7.81 (s, 2H), 7.62 (d, J=8.8 Hz, 2H), 7.27-7.18 (m, 6H), 6.88 (d, J=8.8 Hz, 2H), 6.03 (d, J=7.3 Hz, 1H), 4.53-4.48 (m, 1H), 4.06-4.01 (m, 2H), 3.26 (bd, J=15.9 Hz, 2H), 3.07 (dd, J=16.1, 8.9 Hz, 2H), 2.85 (t, J=6.2 Hz, 2H), 2.51-2.41 (m, 4H), 1.50-1.44 (m, 4H), 0.88 (t, J=7.4 Hz, 6H). ¹³C NMR (126 MHz, CDCl₃) δ 188.0, 166.7, 161.9, 150.8 (dd, J_{CF}=253.2 Hz, J_{C-CF}=12.6 Hz, 2C), 150.5 (dd, J_{CF}=249.4 Hz, J_{C-CF}=12.6 Hz, 2C), 138.0 (2C), 132.8 (2C), 132.2 (dd, J_{C-C-CF}=5.0 Hz, J_{C-C-CF}=3.7 Hz, 2C), 128.7 (2C), 127.2 (dd, J_{C-C-CF}=7.5 Hz, J_{C-C-CF}=3.7 Hz, 2C), 126.1, 119.1 (d, J_{C-CF}=17.6 Hz, 2C), 117.8 (d, J_{C-CF}=17.6 Hz, 2C), 114.5 (2C), 67.1, 57.2, 52.9, 44.6, 33.9, 20.5, 12.0. ¹⁹F NMR (376 MHz, CDCl₃) δ −135.0 (d, J_{FF}=18.8 Hz, 2F), −136.5 (d, J_{FF}=22.5 Hz, 2F). HR-APCI m/z calcd for C₃₅H₃₇F₄N₂O₃ [M+H]=609.2734, found 609.2781.

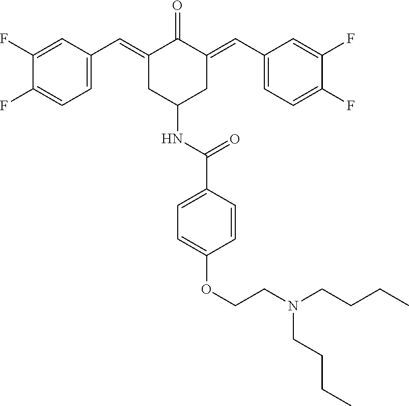 [SMILES: CCCCN(CCCC)CCOc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-(2-(dibutylamino)ethoxy)benzamide (JCS012)

¹H NMR (500 MHz, CDCl₃) δ 7.81 (s, 2H), 7.62 (d, J=8.8 Hz, 2H), 7.27-7.18 (m, 6H), 6.88 (d, J=8.8 Hz, 2H), 6.09-6.04 (m, 1H), 4.53-4.48 (m, 1H), 4.03 (t, J=6.3 Hz, 2H), 3.24 (bd, J=16.7 Hz, 2H), 3.06 (dd, J=16.7, 7.5 Hz, 2H), 2.85 (t, J=6.3 Hz, 2H), 2.54-2.46 (m, 4H), 1.43 (tt, J=7.6, 6.3 Hz, 4H), 1.30 (dt, J=14.8, 7.4 Hz, 4H), 0.90 (t, J=7.4 Hz, 6H). ¹³C NMR (126 MHz, CDCl₃) δ 187.8, 166.5, 161.8, 150.6 (dd, J_{CF}=253.2 Hz, J_{C-CF}=12.6 Hz, 2C), 150.2 (dd, J_{CF}=249.4 Hz, J_{C-CF}=12.6 Hz, 2C), 137.8 (2C), 132.7 (2C), 132.1 (dd, J_{C-C-CF}=6.3 Hz, J_{C-C-CF}=3.7 Hz, 2C), 128.6 (2C), 127.0 (dd, J_{C-C-CF}=6.3 Hz, J_{C-C-CF}=3.7 Hz, 2C), 126.0, 118.9 (d, J_{C-CF}=17.6 Hz, 2C), 117.6 (d, J_{C-CF}=17.6 Hz, 2C), 114.5 (2C), 66.9, 54.7, 52.7, 44.5, 33.4, 29.3, 20.6, 14.0. ¹⁹F NMR (376 MHz, CDCl₃) δ −135.0 (d, J_{FF}=18.8 Hz, 2F), −136.5 (d, J_{FF}=18.8 Hz, 2F). HR-APCI m/z calcd for C₃₇H₄₁F₄N₂O₃ [M+H]=637.3047, found 637.3096.

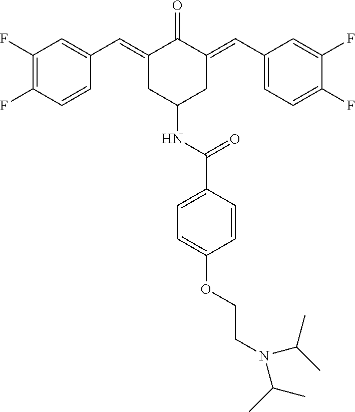 [SMILES: CC(C)N(CCOc1ccc(C(=O)NC2C/C(=C\c3ccc(F)c(F)c3)C(=O)/C(=C/c3ccc(F)c(F)c3)C2)cc1)C(C)C]

### N-(3,5-Bis((E)-3,4-difluorobenzylidene)-4-oxocyclohexyl)-4-(2-(diisopropylamino)ethoxy)benzamide (JCS013)

¹H NMR (500 MHz, CDCl₃) δ 7.76 (s, 2H), 7.63 (d, J=8.8 Hz, 2H), 7.24-7.13 (m, 6H), 6.84 (d, J=8.8 Hz, 2H), 6.29 (d, J=7.2 Hz, 1H), 4.50-4.44 (m, 1H), 3.90 (t, J=6.7 Hz, 2H), 3.24 (bd, J=15.7, Hz, 2H), 3.04 (m, 4H), 2.81 (t, J=7.1 Hz, 2H), 1.03 (d, J=6.5 Hz, 12H). ¹³C NMR (126 MHz, CDCl₃) δ 187.9, 166.7, 161.9, 150.7 (dd, J_{CF}=253.2 Hz, J_{C-CF}=12.6 Hz, 2C), 150.2 (dd, J_{CF}=249.4 Hz, J_{C-CF}=12.6 Hz, 2C), 137.8 (2C), 132.8 (2C), 132.2 (dd, J_{C-C-CF}=5.0 Hz, J_{C-C-CF}=3.7 Hz, 2C), 128.6 (2C), 127.1 (dd, J_{C-C-CF}=7.5 Hz, J_{C-C-CF}=3.7 Hz, 2C), 126.0, 119.0 (d, J_{C-CF}=17.6 Hz, 2C), 117.7 (d, J_{C-CF}=17.6 Hz, 2C), 114.5 (2C), 69.4, 49.8, 44.6, 44.3, 33.8, 20.8. ¹⁹F NMR (376 MHz, CDCl₃) δ −135.0 (d, J_{FF}=22.5 Hz, 2F), −136.5 (d, J_{FF}=22.5 Hz, 2F). HR-APCI m/z calcd for C₃₅H₃₇F₄N₂O₃ [M+H]=609.2734, found 609.2777.

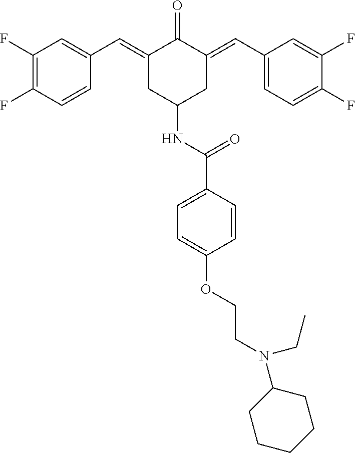 [SMILES: CCN(CCOc1ccc(C(=O)NC2C/C(=C\c3ccc(F)c(F)c3)C(=O)/C(=C/c3ccc(F)c(F)c3)C2)cc1)C1CCCCC1]

### N-(3,5-Bis((E)-3,4-difluorobenzylidene)-4-oxocyclohexyl)-4-(2-(cyclohexyl(ethyl)amino)ethoxy)-benzamide (JCS014)

¹H NMR (500 MHz, CDCl₃) δ 7.80 (s, 2H), 7.62 (d, J=8.3 Hz, 2H), 7.28-7.17 (m, 6H), 6.87 (d, J=8.3 Hz, 2H), 6.11 (d, J=6.3 Hz, 1H), 4.55-4.45 (m, 1H), 4.01 (t, J=6.7 Hz, 2H), 3.26 (bd, J=15.1 Hz, 2H), 3.06 (dd, J=15.4, 8.1 Hz, 2H), 2.89 (t, J=6.0 Hz, 2H), 2.75-2.62 (m, 1H), 2.59-2.53 (m, 1H), where is there a 1H singlet 1.91-1.74 (m, 4H), 1.64-1.62 (m, 1H), 1.27-1.18 (m, 4H), 1.12-1.04 (m, 1H). ¹³C NMR (126 MHz, CDCl₃) δ 188.0, 166.7, 161.8, 150.8 (dd, J_{CF}=253.2 Hz, J_{C-CF}=12.6 Hz, 2C), 150.3 (dd, J_{CF}=249.4 Hz, J_{C-CF}=12.6 Hz, 2C), 137.9 (2C), 132.8 (2C), 132.2 (dd, J_{C-C-CF}=6.3 Hz, J_{C-C-CF}=3.7 Hz, 2C), 128.8 (2C), 127.2 (dd, J_{C-C-CF}=6.3 Hz, J_{C-C-CF}=2.5 Hz, 2C), 126.1, 119.0 (d, J_{C-CF}=17.6 Hz, 2C), 117.7 (d, J_{C-CF}=17.6 Hz, 2C), 114.5 (2C), 68.2, 61.1, 48.9, 46.0, 44.6, 33.9, 29.2, 26.3, 26.2, 14.0. ¹⁹F NMR (376 MHz, CDCl₃) δ −135.0 (d, J_{FF}=22.5 Hz, 2F), −136.5 (d, J_{FF}=22.5 Hz, 2F). HR-APCI m/z calcd for C₃₇H₃₉F₄N₂O₃ [M+H]=635.2891, found 635.2950.

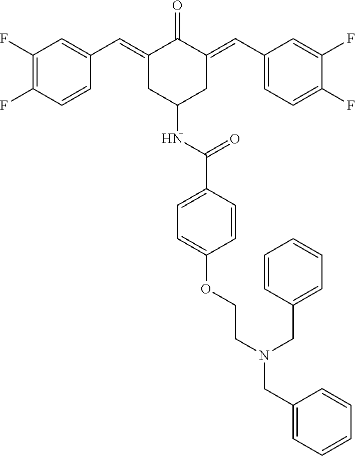 [SMILES: O=C1/C(=C/c2ccc(F)c(F)c2)CC(NC(=O)c2ccc(OCCN(Cc3ccccc3)Cc3ccccc3)cc2)C/C1=C\c1ccc(F)c(F)c1]

### N-(3,5-Bis((E)-3,4-difluorobenzylidene)-4-oxocyclohexyl)-4-(2-(dibenzylamino)ethoxy)benzamide (JCS015)

¹H NMR (500 MHz, CDCl₃) δ 7.78 (s, 2H), 7.62 (d, J=8.7 Hz, 2H), 7.39 (d, J=7.4 Hz, 4H), 7.32 (t, J=7.5 Hz, 4H), 7.29-7.14 (m, 8H), 6.78 (d, J=8.6 Hz, 2H), 6.26 (d, J=7.0 Hz, 1H), 4.52-4.46 (m, 1H), 4.01 (t, J=6.0 Hz, 2H), 3.71 (s, 4H), 3.25 (bd, J=16.4 Hz, 2H), 3.06 (dd, J=15.9, 8.3 Hz, 2H), 2.90 (t, J=6.0 Hz, 2H). ¹³C NMR (126 MHz, CDCl₃) δ 187.9, 166.7, 161.7, 150.7 (dd, J_{CF}=253.2 Hz, J_{C-CF}=12.6 Hz, 2C), 150.2 (dd, J_{CF}=249.4 Hz, J_{C-CF}=12.6 Hz, 2C), 139.5 (2C), 137.9 (2C), 132.8 (2C), 132.2 (dd, J_{C-C-CF}=6.3 Hz, J_{C-C-CF}=3.7 Hz, 2C), 128.8 (4C), 128.3 (2C), 127.1 (dd, J_{C-C-CF}=6.3 Hz, J_{C-C-CF}=3.7 Hz, 2C), 126.1, 119.0 (d, J_{C-CF}=17.6 Hz, 2C), 117.7 (d, J_{C-CF}=17.6 Hz, 2C), 114.3 (2C), 66.9, 59.2, 52.0, 44.6, 33.8. ¹⁹F NMR (376 MHz, CDCl₃) δ −135.0 (d, J_{FF}=22.5 Hz, 2F), −136.4 (d, J_{FF}=22.5 Hz, 2F). HR-APCI m/z calcd for C₄₃H₃₈F₄N₂O₃ [M+H]=705.2734, found 705.2749.

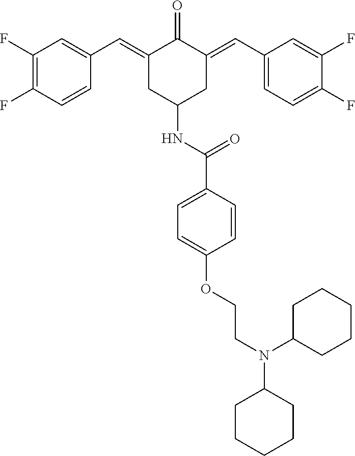 [SMILES: O=C1/C(=C/c2ccc(F)c(F)c2)CC(NC(=O)c2ccc(OCCN(C3CCCCC3)C3CCCCC3)cc2)C/C1=C\c1ccc(F)c(F)c1]

### N-(3,5-Bis((E)-3,4-difluorobenzylidene)-4-oxocyclohexyl)-4-(2-(dicyclohexylamino)ethoxy)benz-amide (JCS017)

¹H NMR (500 MHz, CDCl₃) δ 7.81 (s, 2H), 7.62 (d, J=8.7 Hz, 2H), 7.26-7.17 (m, 6H), 6.87 (d, J=8.7 Hz, 2H), 6.05 (d, J=7.3 Hz, 1H), 4.53-4.48 (m, 1H), 3.87 (t, J=7.3 Hz, 2H), 3.24 (bd, J=15.7, Hz, 2H), 3.06 (ddd, J=15.9, 8.3, 2.2 Hz, 2H), 2.92 (t, J=3.7 Hz, 2H), 2.57 (t, J=3.7 Hz, 2H), 1.77-1.73 (m, 8H), 1.62-1.59 (m, 2H), 1.26-1.21 (m, 8H), 1.10-1.03 (m, 2H). ¹³C NMR (126 MHz, CDCl₃)¹³C NMR (126 MHz, Chloroform-d) δ 188.0, 166.7, 162.0, 150.8 (dd, J_{CF}=253.2 Hz, J_{C-CF}=12.6 Hz, 2C), 150.3 (dd, J_{CF}=249.4 Hz, J_{C-CF}=12.6 Hz, 2C), 137.9 (2C), 132.8 (2C), 132.2 (dd, J_{C-C-CF}=6.3 Hz, J_{C-C-CF}=5.0 Hz, 2C), 128.8 (2C), 127.2 (dd, J_{C-C-CF}=6.3 Hz, J_{C-C-CF}=2.5 Hz, 2C), 125.9, 119.0 (d, J_{C-CF}=17.6 Hz, 2C), 117.7 (d, J_{C-CF}=17.6 Hz, 2C), 114.4 (2C), 69.7, 59.0, 45.5, 44.6, 33.9, 31.9, 26.3 (2C). ¹⁹F NMR (376 MHz, CDCl₃) δ −135.0 (d, J_{FF}=18.8 Hz, 2F), −136.4 (d, J_{FF}=22.5 Hz, 2F). HR-APCI m/z calcd for C₄₁H₄₅F₄N₂O₃ [M+H]=689.3371, found 689.3375.

### General Procedure B:

The compounds were generally prepared by reaction of the corresponding aldehydes, e.g., 3,4-difluorobenzaldehyde, with tert-butyl (4-oxocyclohexyl)carbamate in the present of 20% aq. sodium hydroxide to give N-tert-butyl (3,5-bis((E)-3,4-difluorobenzylidene)-4-oxocyclohexyl)carbamate. Treatment of the product with trifluoroacetic acid (TFA) caused deprotection of the Boc group. Benzoylation of 4-amino-2,6-bis((E)-3,4-difluorobenzylidene)cyclohexan-1-one with 4-(2-(propylamino)-ethoxy)benzoic acid using standard peptide coupling reagents TBTU or EDC and HOAt afforded the N-(3,5-bis((E)-3,4-difluorobenzylidene)-4-oxocyclohexyl)-4-(2-(propylamino)ethoxy)benzamide.

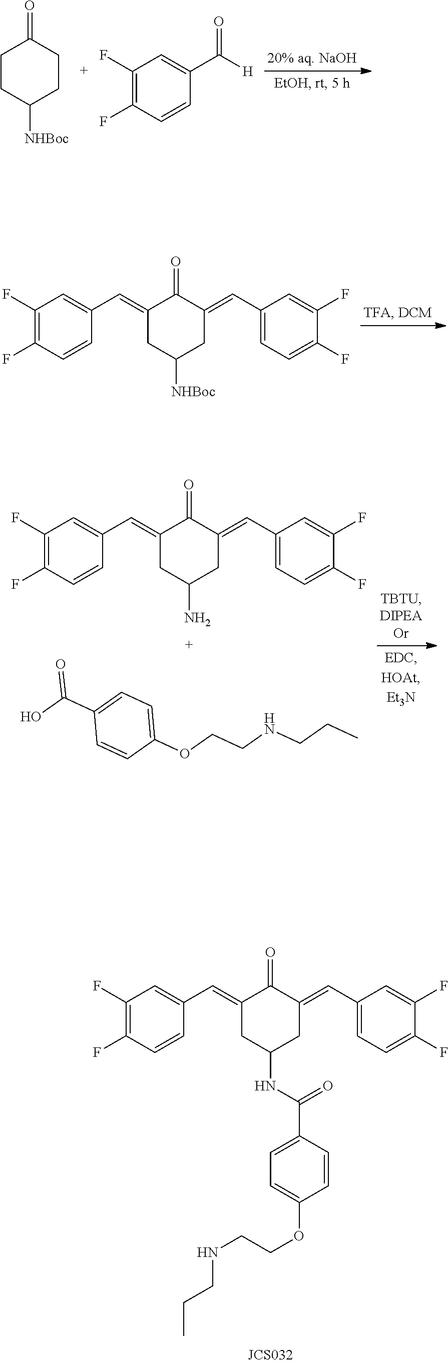

### Example JCSO32

To the mixture of the N-tert-butyl (4-oxocyclohexyl)carbamate (213.28 mg, 1 mmol, 1.0 equiv.) and ethanol (1.0 mL) in a round bottom flask added drop-wise 20% aqueous sodium hydroxide (1.5 mL) and stirred for five minutes. To this mixture was added 3,4-difluorobenzaldehyde (355.3 mg, 2.5 mmol, 2.5 equiv.). The reaction mixture was then allowed to stir at 21° C. for 5 h. After 5 h the yellow precipitate thus obtained was filtered, washed with water, cold ethanol and dried to get the pure product (360 mg, 78% yield).

Trifluoroacetic acid (0.5 ml) was added to a solution of N-tert-butyl (3,5-bis((E)-3,4-difluorobenzylidene)-4-oxocyclohexyl)carbamate (230.7 mg, 0.5 mmol) in dichloromethane (5.0 ml) at 21° C. and stirred overnight at 21° C. Then, the solvent of the reaction solution was distilled off under reduced pressure and the resulting residue was poured into a 1N aqueous sodium hydroxide solution and extracted with ethyl acetate. The organic layer was washed with a saturated aqueous sodium chloride solution and then dried over anhydrous magnesium sulfate. The solvent was distilled off under reduced pressure to obtain 4-amino-2,6-bis((E)-3,4-difluorobenzylidene)cyclohexan-1-one.

The mixture of 4-amino-2,6-bis((E)-3,4-difluorobenzylidene)cyclohexan-1-one (180.7 mg, 0.5 mmol, 1.0 equiv.) and anhydrous diisopropylethylamine (261.2 μL, 1.5 mmol, 3.0 equiv.) in THF was maintained at 0° C. (ice bath). To this cooled mixture, 4-(2-(propylamino)ethoxy)benzoic acid (111.6 mg, 0.5 mmol, 1.0 equiv.) in 2.0 mL THE was added drop wise followed by and TBTU (240.8 mg, 0.75 mmol, 1.5 equiv.). After the complete addition of 4-(2-(ethylamino)ethoxy)benzoic acid, the reaction mixture was slowly warmed up to 21° C. and stirred overnight. After completion of the reaction solvent was evaporated and the residue was stirred in sat aqueous NaHCO₃ for 5 min. The mixture was extracted with ethyl acetate three times. The organic layer was washed with a saturated aqueous sodium chloride solution and then dried over anhydrous sodium sulfate. The solvent was evaporated, followed by flash chromatography (gradient elution 10% methanol/ethyl acetate-75% methanol/ethyl acetate) to give the desired product JCSO32 (222.2 mg, 75% yield) as yellow solid.

### Synthesis of 4-(2-(propylamino)ethoxy)benzoic acid

Methyl 4-hydroxybenzoate (5.0 g, 32.86 mmol, 10 equiv) was combined with 1,2-dibromoethane (35 mL) and potassium carbonate (6.8 g, 49.23 mmol, 1.5 equiv) and the mixture was heated at reflux for 18 h. The reaction mixture was concentrated under reduced pressure and then the residue was partitioned between ethyl ether (300 mL) and water (200 mL). The ether layer was extracted with 2 N sodium hydroxide (5×30 mL). The solvent was removed to give the desired product as a white solid (8.5 g, 99% yield).

To a round bottom flask equipped with a stir-bar was added a solution of methyl 4-(2-bromoethoxy)benzoate (2.591 g, 10 mmol, 1.0 equiv), dimethylformamide (DMF, 20 mL), cesium carbonate (9.7 g, 30 mmol, 3.0 equiv) and propylamine (1.7 g, 30 mmol, 3.0 equiv). The mixture was stirred at 21° C. for 24 h, after which time ethyl acetate (200 mL) and water (200 mL) were added. The organic layer was washed with water three times and then dried over sodium sulfate. The pure product was obtained by flash chromatography on silica gel (gradient elution 10% methanol/EtOAc-50% methanol/EtOAc) to give the desired product, methyl 4-(2-(propylamino)ethoxy)benzoate (1.4 g, 60% yield), as a light yellow oil.

Methyl 4-(2-(propylamino)ethoxy)benzoate (1.18 g, 5.0 mmol, 1.0 equiv) was dissolved in 2.5 mL ethanol and added to a solution of sodium hydroxide (0.4 g) in 2.5 mL water. The mixture was heated under reflux for 2 h. The ethanol was removed in vacuo and the aqueous solution was acidified with cone. HCl at 5° C. The solid was collected, treated with cold water, filtered and dried at 55-60° C. in vacuo to give 4-(2-(propylamino)ethoxy)benzoic acid hydrochloride as white sold (1.16 g, 90% yield) which was pure enough to be used for next step.

The following compounds were synthesized by B: JCS018, JCS019, JCS20, JCS021, JCS022, JCS023, JCS024, JCS025, JCS026, JCS061, JCS062, JCS064, JCS068, JCS029, JCS030, JCS031, JCS032, JCS033, JCS034, JCS035, JCS036, JCS037, JCS0038, JCS039, JCSO40, JCSO45, JCSO49, JCS77, JCS078, JCS079, JCS080, JCS081, JCS083, JCS084.

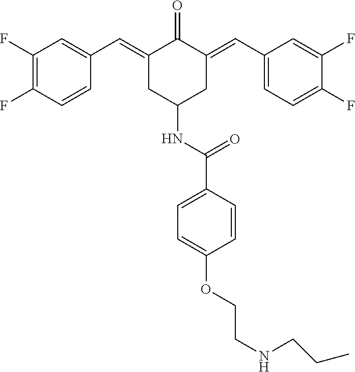 [SMILES: CCCNCCOc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-(2-(propylamino)ethoxy)benzamide (JCSO32)

¹H NMR (500 MHz, CDCl₃) δ 7.80 (s, 2H), 7.62 (d, J=8.8 Hz, 2H), 7.26-7.16 (m, 6H), 6.88 (d, J=8.8 Hz, 2H), 6.17 (d, J=7.3 Hz, 1H), 4.50-4.47 (m, 1H), 4.12 (t, J=5.2 Hz, 2H), 3.26 (bd, J=15.7 Hz, 2H), 3.11-3.02 (m, 4H), 2.68 (t, J=5.0 Hz, 2H), 2.21 (s, 1H), 1.62-1.54 (m, 2H), 0.93 (t, J=7.4 Hz, 3H). ¹³C NMR (126 MHz, CDCl₃) δ 187.9, 166.7, 161.6, 150.8 (dd, J_{CF}=253.2 Hz, J_{C-CF}=12.6 Hz, 2C), 150.3 (dd, J_{CF}=250.7 Hz, J_{C-CF}=13.8 Hz, 2C), 137.9 (2C), 132.8 (2C), 132.2 (dd, J_{C-C-CF}=6.3 Hz, J_{C-C-CF}=3.7 Hz, 2C), 128.8 (2C), 127.2 (dd, J_{C-C-CF}=7.5 Hz, J_{C-C-CF}=3.7 Hz, 2C), 126.5, 119.0 (d, J_{C-CF}=15.1 Hz, 2C), 117.8 (d, J_{C-CF}=17.6 Hz, 2C), 114.4 (2C), 67.2, 51.6, 48.4, 44.7, 33.8, 22.8, 11.8. ¹⁹F NMR (376 MHz, CDCl₃) δ −135.0 (d, J_{FF}=22.5 Hz, 2F), −136.5 (d, J_{FF}=22.5 Hz, 2F). HR-APCI m/z calcd for C₃₂H₃₁F₄N₂O₃ [M+H]=567.2259, found 567.2268.

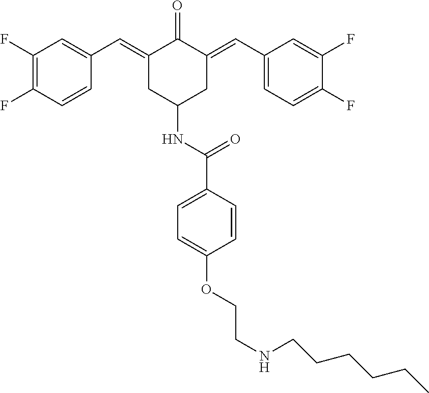 [SMILES: CCCCCCNCCOc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-(2-(hexylamino)ethoxy)benzamide (JCSO29)

¹H NMR (500 MHz, DMSO-d₆) δ 9.33 (bs, 2H), 8.60 (d, J=6.5 Hz, 1H), 7.87 (d, J=8.8 Hz, 2H), 7.68 (m, 2H), 7.59-7.47 (m, 2H), 7.48-7.37 (m, 2H), 7.04 (d, J=8.8 Hz, 2H), 4.35 (t, J=5.2 Hz, 2H), 4.10-4.05 (m, 1H), 3.31 (t, J=4.4 Hz, 2H), 3.18 (bd, J=16.1 Hz, 2H), 3.06 (dd, J=15.9, 10.9 Hz, 2H), 2.93 (t, J=8.1 Hz, 2H), 1.70-1.63 (m, 2H), 1.36-1.19 (m, 6H), 0.86 (t, J=6.6 Hz, 3H). ¹³C NMR (126 MHz, DMSO-d₆) δ 187.6, 165.4, 160.1, 149.5 (dd, J_{CF}=249.4 Hz, J_{C-CF}=12.6 Hz, 2C), 149.2 (dd, J_{CF}=245.7 Hz, J_{C-CF}=12.6 Hz, 2C), 135.1 (2C), 134.7 (2C), 132.7 (dd, J_{C-C-CF}=6.3 Hz, J_{C-C-CF}=3.7 Hz, 2C), 129.3 (2C), 127.6 (dd, J_{C-C-CF}=6.3 Hz, J_{C-C-CF}=3.7 Hz, 2C), 126.9, 119.1 (d, J_{C-CF}=17.6 Hz, 2C), 117.8 (d, J_{C-CF}=17.6 Hz, 2C), 114.0 (2C), 63.4, 47.1, 45.6, 45.0, 33.1, 30.7, 25.6, 25.2, 21.8, 13.8. ¹⁹F NMR (376 MHz, DMSO-d₆) δ −136.9 (d, J_{FF}=22.5 Hz, 2F), −137.9 (d, J_{FF}=22.5 Hz, 2F). HR-APCI m/z calcd for C₃₅H₃₇F₄N₂O₃ [M+H]=609.2734, found 609.2735.

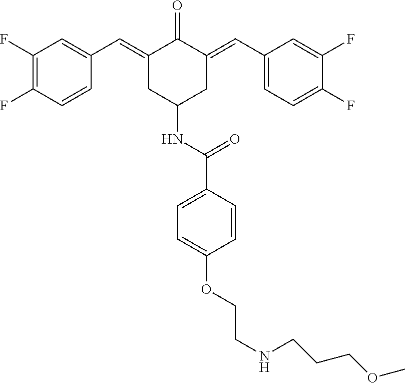 [SMILES: COCCCNCCOc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-(2-((3-methoxypropyl)amino)ethoxy)-benzamide (JCSO30)

¹H NMR (500 MHz, CDCl₃) δ 7.76 (s, 2H), 7.63 (d, J=8.9 Hz, 2H), 7.25-7.14 (m, 6H), 6.85 (d, J=8.8 Hz, 2H), 6.28 (d, J=7.3 Hz, 1H), 4.49-4.44 (m, 1H), 4.08 (t, J=5.2 Hz, 2H), 3.45 (t, J=6.1 Hz, 2H), 3.31 (s, 3H), 3.24 (d, J=15.0 Hz, 2H), 3.09-2.97 (m, 4H), 2.77 (t, J=7.0 Hz, 2H), 2.53 (bs, 1H), 1.82-1.75 (m, 2H). ¹³C NMR (126 MHz, CDCl₃) δ 187.9, 166.6, 161.7, 150.7 (dd, J_{CF}=253.2 Hz, J_{C-CF}=12.6 Hz, 2C), 150.2 (dd, J_{CF}=250.7 Hz, J_{C-CF}=13.8 Hz, 2C), 137.8 (2C), 132.8 (2C), 132.2 (dd, J_{C-C-CF}=6.3 Hz, J_{C-C-CF}=3.7 Hz, 2C), 128.8 (2C), 127.1 (dd, J_{C-C-CF}=7.5 Hz, J_{C-C-CF}=3.7 Hz, 2C), 126.4, 119.0 (d, J_{C-CF}=17.6 Hz, 2C), 117.7 (d, J_{C-CF}=17.6 Hz, 2C), 114.3 (2C), 71.3, 67.4, 58.7, 48.62, 47.8, 44.7, 33.8, 29.8. ¹⁹F NMR (376 MHz, CDCl₃) δ −131.1 (d, J_{FF}=30.0 Hz, 2F), −129.7 (d, J_{FF}=30.0 Hz, 2F). HR-APCI m/z calcd for C₃₃H₃₃F₄N₂O₃ [M+H]=597.2370, found 597.2366.

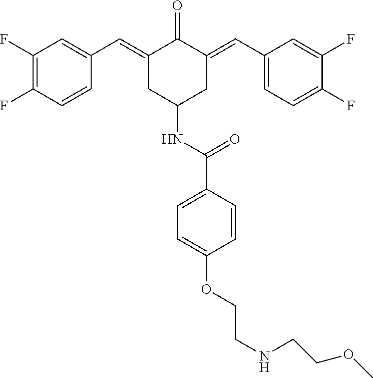 [SMILES: COCCNCCOc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-(2-((2-methoxyethyl)amino)ethoxy)-benzamide (JCSO31)

¹H NMR (500 MHz, CDCl₃) δ 7.75 (s, 2H), 7.62 (d, J=8.8 Hz, 2H), 7.24-7.11 (m, 6H), 6.85 (d, J=8.8 Hz, 2H), 6.32 (d, J=7.3 Hz, 1H), 4.50-4.42 (m, 1H), 4.07 (t, J=5.2 Hz, 2H), 3.50 (t, J=5.0 Hz, 2H), 3.34 (s, 3H), 3.23 (bd, J=15.0 Hz, 2H), 3.09-2.98 (m, 4H), 2.85 (t, J=5.0 Hz, 2H), 2.35 (bs, 1H). ¹³C NMR (126 MHz, CDCl₃) δ 187.9, 166.7, 161.7, 150.6 (dd, J_{CF}=253.2 Hz, J_{C-CF}=12.6 Hz, 2C), 150.2 (dd, J_{CF}=249.4 Hz, J_{C-CF}=13.8 Hz, 2C), 137.8 (2C), 132.8 (2C), 132.2 (dd, J_{C-C-CF}=6.3 Hz, J_{C-C-CF}=3.7 Hz, 2C), 128.8 (2C), 127.1 (dd, J_{C-C-CF}=7.5 Hz, J_{C-C-CF}=3.7 Hz, 2C), 126.3, 118.9 (d, J_{C-CF}=17.6 Hz, 2C), 117.7 (d, J_{C-CF}=17.6 Hz, 2C), 114.3 (2C), 71.9, 67.6, 58.9, 49.3, 48.6, 44.7, 33.8. ¹⁹F NMR (376 MHz, CDCl₃) δ −135.1 (d, J_{FF}=22.5 Hz, 2F), −136.5 (d, J_{FF}=22.5 Hz, 2F). HR-APCI m/z calcd for C₃₂H₃₁F₄N₂O₃ [M+H]=583.2210, found 583.2212.

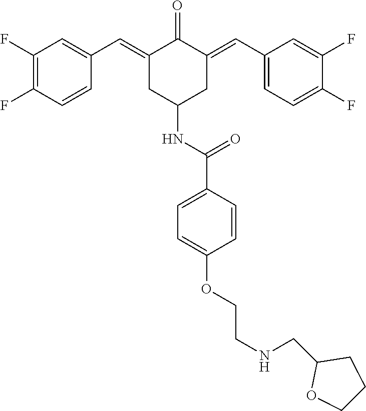 [SMILES: O=C1/C(=C/c2ccc(F)c(F)c2)CC(NC(=O)c2ccc(OCCNCC3CCCO3)cc2)C/C1=C\c1ccc(F)c(F)c1]

### N-(3,5-Bis((E)-3,4-difluorobenzylidene)-4-oxocyclohexyl)-4-(2-(((tetrahydrofuran-2-yl)methyl)-amino)ethoxy)benzamide (JCSO33)

¹H NMR (500 MHz, CDCl₃) δ 7.76 (s, 2H), 7.62 (d, J=8.8 Hz, 2H), 7.25-7.14 (m, 6H), 6.85 (d, J=8.8 Hz, 2H), 6.30 (d, J=7.3 Hz, 1H), 4.49-4.43 (m, 1H), 4.07 (t, J=5.2 Hz, 2H), 4.02-3.97 (m, 1H), 3.85-3.70 (m, 2H), 3.08-2.98 (m, 4H), 2.80-2.67 (m, 2H), 2.41 (bs, 1H), 2.00-1.94 (m, 1H), 1.90-1.83 (m, 2H), 1.56-1.50 (m, 1H). ¹³C NMR (126 MHz, CDCl₃) δ 187.9, 166.7, 161.7, 150.7 (dd, J_{CF}=254.5 Hz, J_{C-CF}=12.6 Hz, 2C), 150.2 (dd, J_{CF}=250.7 Hz, J_{C-CF}=13.8 Hz, 2C), 137.8 (2C), 132.8 (2C), 132.2 (dd, J_{C-C-CF}=6.3 Hz, J_{C-C-CF}=3.7 Hz, 2C), 128.8 (2C), 127.1 (dd, J_{C-C-CF}=7.5 Hz, J_{C-C-CF}=3.7 Hz, 2C), 126.3, 119.0 (d, J_{C-CF}=17.6 Hz, 2C), 117.7 (d, J_{C-CF}=17.6 Hz, 2C), 114.3 (2C), 78.3, 68.0, 67.7, 54.4, 48.8, 44.6, 33.8, 29.4, 25.8. ¹⁹F NMR (376 MHz, CDCl₃) δ −135.1 (d, J_{FF}=22.5 Hz, 2F), −136.5 (d, J_{FF}=22.5 Hz, 2F). HR-APCI m/z calcd for C₃₄H₃₃F₄N₂O₄ [M+H]=609.2371, found 609.2371.

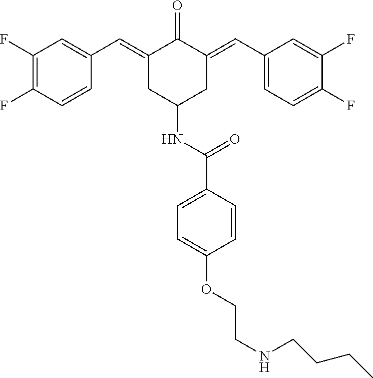 [SMILES: CCCCNCCOc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-(2-(butylamino)ethoxy)benzamide (JCSO34)

¹H NMR (500 MHz, CDCl₃) δ 7.78 (s, 2H), 7.62 (d, J=8.8 Hz, 2H), 7.25-7.15 (m, 6H), 6.87 (d, J=8.8 Hz, 2H), 6.20 (d, J=7.2 Hz, 1H), 4.52-4.45 (m, 1H), 4.10 (t, J=5.2 Hz, 2H), 3.25 (bd, J=15.0 Hz, 2H), 3.08-3.01 (m, 4H), 2.70 (t, J=7.3 Hz, 2H), 2.34 (bs, 1H), 1.41-1.32 (m, 2H), 1.40-1.33 (m, 2H), 0.91 (t, J=7.3 Hz, 3H). ¹³C NMR (126 MHz, CDCl₃) δ 187.9, 166.7, 161.7, 150.7 (dd, J_{CF}=254.5 Hz, J_{C-CF}=12.6 Hz, 2C), 150.3 (dd, J_{CF}=249.4 Hz, J_{C-CF}=13.8 Hz, 2C), 137.9 (2C), 132.8 (2C), 132.2 (dd, J_{C-C-CF}=6.3 Hz, J_{C-C-CF}=3.7 Hz, 2C), 128.8 (2C), 127.2 (dd, J_{C-C-CF}=7.5 Hz, J_{C-C-CF}=3.7 Hz, 2C), 126.4, 119.0 (d, J_{C-CF}=17.6 Hz, 2C), 117.7 (d, J_{C-CF}=17.6 Hz, 2C), 114.4 (2C), 67.4, 49.6, 48.5, 44.7, 33.8, 29.8, 20.5, 14.0. ¹⁹F NMR (376 MHz, CDCl₃) δ −135.0 (d, J_{FF}=18.8 Hz, 2F), −136.5 (d, J_{FF}=22.5 Hz, 2F). HR-APCI m/z calcd for C₃₃H₃₃F₄N₂O₃ [M+H]=581.2421, found 581.2429.

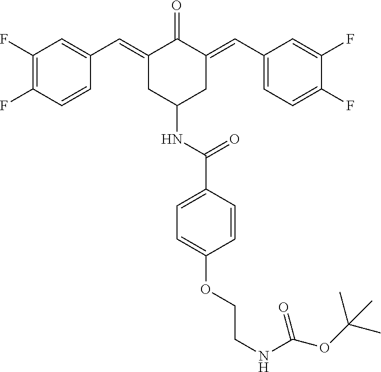 [SMILES: CC(C)(C)OC(=O)NCCOc1ccc(C(=O)NC2C/C(=C\c3ccc(F)c(F)c3)C(=O)/C(=C/c3ccc(F)c(F)c3)C2)cc1]

### tert-Butyl (2-(4-((3,5-bis((E)-3,4-difluorobenzylidene)-4-oxocyclohexyl)carbamoyl)phenoxy)ethyl)-carbamate (JCSO35)

¹H NMR (500 MHz, CDCl₃) δ 7.81 (s, 2H), 7.63 (d, J=8.7 Hz, 2H), 7.26-7.17 (m, 6H), 6.87 (d, J=8.6 Hz, 2H), 6.09 (d, J=7.0 Hz, 1H), 4.97 (bs, 1H), 4.53-4.47 (m, 1H), 4.03 (t, J=5.2 Hz, 2H), 3.53 (q, J=5.5 Hz, 2H), 3.26 (bd, J=15.0 Hz, 2H), 3.07 (dd, J=15.0, 5.0 Hz, 2H), 1.44 (s, 9H). ¹³C NMR (126 MHz, CDCl₃) δ 187.9, 166.6, 161.5, 155.9, 150.8 (dd, J_{CF}=253.6 Hz, J_{C-CF}=12.6 Hz, 2C), 150.3 (dd, J_{CF}=249.4 Hz, J_{C-CF}=13.8 Hz, 2C), 138.0 (2C), 132.8 (2C), 132.2 (dd, J_{C-C-CF}=5.0 Hz, J_{C-C-CF}=3.7 Hz, 2C), 128.9 (2C), 127.2 (dd, J_{C-C-CF}=7.5 Hz, J_{C-C-CF}=3.7 Hz, 2C), 126.6, 119.1 (d, J_{C-CF}=17.6 Hz, 2C), 117.8 (d, J_{C-CF}=17.6 Hz, 2C), 114.4 (2C), 79.8, 67.4, 44.6, 40.0, 33.87, 28. ¹⁹F NMR (376 MHz, CDCl₃) δ −131.1 (d, J_{FF}=30.0 Hz, 2F),−129.7 (d, J_{FF}=26.3 Hz, 2F). HR-APCI m/z calcd for C₃₄H₃₂F₄N₂O₅K [M+K]=663.1878, found 663.1884.

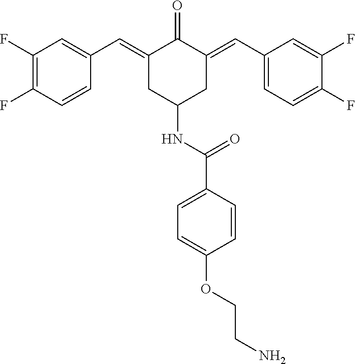 [SMILES: NCCOc1ccc(C(=O)NC2C/C(=C\c3ccc(F)c(F)c3)C(=O)/C(=C/c3ccc(F)c(F)c3)C2)cc1]

### 4-(2-Aminoethoxy)-((E)-3,4-difluorobenzylidene)-4-oxocyclohexyl)benzamide (JCSO36)

¹H NMR (500 MHz, CDCl₃) δ 7.80 (s, 2H), 7.63 (d, J=8.8 Hz, 2H), 7.26-7.16 (m, 6H), 6.89 (d, J=8.8 Hz, 2H), 6.12 (d, J=7.3 Hz, 1H), 4.52-4.47 (m, 1H), 4.00 (t, J=5.2 Hz, 2H), 3.25 (bd, J=15.0 Hz, 2H), 3.13-3.01 (m, 4H), 1.49 (s, 2H). ¹³C NMR (126 MHz, CDCl₃) δ 188.0, 166.6, 161.9, 150.8 (dd, J_{CF}=254.5 Hz, J_{C-CF}=12.6 Hz, 2C), 150.3 (dd, J_{CF}=249.4 Hz, J_{C-CF}=13.8 Hz, 2C), 138.0 (2C), 132.8 (2C), 132.2 (dd, J_{C-C-CF}=5.0 Hz, J_{C-C-CF}=3.7 Hz, 2C), 128.9 (2C), 127.2 (dd, J_{C-C-CF}=6.3 Hz, J_{C-C-CF}=2.5 Hz, 2C), 126.4, 119.0 (d, J_{C-CF}=17.6 Hz, 2C), 117.7 (d, J_{C-CF}=16.3 Hz, 2C), 114.4 (2C), 70.4, 44.6, 41.5, 33.8. ¹⁹F NMR (376 MHz, CDCl₃) δ −135.0 (d, J_{FF}=22.5 Hz, 2F), −136.5 (d, J_{FF}=22.5 Hz, 2F). HR-APCI m/z calcd for C₃₉H₂₅F₄N₂O₃ [M+H]=525.1795, found 525.1799.

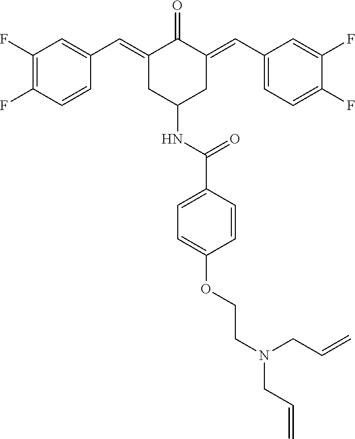 [SMILES: C=CCN(CC=C)CCOc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-(2-(diallylamino)ethoxy)benzamide (JCSO37)

¹H NMR (500 MHz, CDCl₃) δ 7.80 (s, 2H), 7.62 (d, J=8.4 Hz, 2H), 7.28-7.16 (m, 6H), 6.87 (d, J=8.4 Hz, 2H), 6.10 (d, J=7.3 Hz, 1H), 5.92-5.84 (m, 2H), 5.23-5.15 (m, 4H), 4.53-4.47 (m, 1H), 4.07 (t, J=6.0 Hz, 2H), 3.35-3.15 (m, 6H), 3.06 (dd, J=16.1, 8.1 Hz, 2H), 2.89 (t, J=6.0 Hz, 2H). ¹³C NMR (126 MHz, CDCl₃) δ 188.0, 166.6, 161.7, 150.8 (dd, J_{CF}=254.5 Hz, J_{C-CF}=12.6 Hz, 2C), 150.3 (dd, J_{CF}=249.4 Hz, J_{C-CF}=13.8 Hz, 2C), 137.9 (2C), 135.2 (2C), 132.8 (2C), 132.2 (dd, J_{C-C-CF}=6.3 Hz, J_{C-C-CF}=3.7 Hz, 2C), 128.8 (2C), 127.2 (dd, J_{C-C-CF}=6.3 Hz, J_{C-C-CF}=2.5 Hz, 2C), 126.2, 119.1 (d, J_{C-CF}=17.6 Hz, 2C), 118.2 (2C), 117.7 (d, J_{C-CF}=16.3 Hz, 2C), 114.4 (2C), 66.7, 57.8, 51.8, 44.6, 33.8. ¹⁹F NMR (376 MHz, CDCl₃) δ −131.1 (d, J_{FF}=26.3 Hz, 2F), −129.7 (d, J_{FF}=26.3 Hz, 2F). HR-APCI m/z calcd for C₃₅H₃₃F₄N₂O₃ [M+H]=605.2421, found 605.2423.

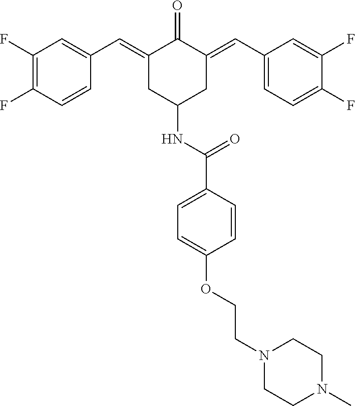 [SMILES: CN1CCN(CCOc2ccc(C(=O)NC3C/C(=C\c4ccc(F)c(F)c4)C(=O)/C(=C/c4ccc(F)c(F)c4)C3)cc2)CC1]

### N-(3,5-Bis((E)-3,4-difluorobenzylidene)-4-oxocyclohexyl)-4-(2-(4-methylpiperazin-1-yl)ethoxy)benz-amide (JCSO38)

¹H NMR (500 MHz, CDCl₃) δ 7.73 (s, 2H), 7.62 (d, J=8.8 Hz, 2H), 7.23-7.10 (m, 6H), 6.84 (d, J=8.8 Hz, 2H), 6.30 (d, J=7.2 Hz, 1H), 4.48-4.42 (m, 1H), 4.08 (t, J=5.8 Hz, 2H), 3.22 (bd, J=15.3 Hz, 2H), 3.02 (dd, J=15.8, 8.4 Hz, 2H), 2.78 (t, J=5.8 Hz, 2H), 2.66-2.52 (m, 4H), 2.50-2.35 (m, 4H), 2.26 (s, 3H). ¹³C NMR (126 MHz, CDCl₃) δ 187.8, 166.6, 161.6, 150.6 (dd, J_{CF}=253.2 Hz, J_{C-CF}=12.6 Hz, 2C), 150.2 (dd, J_{CF}=249.4 Hz, J_{C-CF}=13.8 Hz, 2C), 137.7 (2C), 132.8 (2C), 132.1 (dd, J_{C-C-CF}=6.3 Hz, J_{C-C-CF}=3.7 Hz, 2C), 128.8 (2C), 127.1 (dd, J_{C-C-CF}=6.3 Hz, J_{C-C-CF}=3.7 Hz, 2C), 126.2, 118.9 (d, J_{C-CF}=17.6 Hz, 2C), 117.7 (d, J_{C-CF}=16.3 Hz, 2C), 114.4 (2C), 66.2, 57.0, 55.0, 53.6, 46.1, 44.6, 33.7. ¹⁹F NMR (376 MHz, CDCl₃) δ 131.1 (d, J_{FF}=30.0 Hz, 2F), 129.7 (d, J_{FF}=26.3 Hz, 2F). HR-APCI m/z calcd for C₃₅H₃₄F₄N₃O₃ [M+H]=608.2530, found 608.2539.

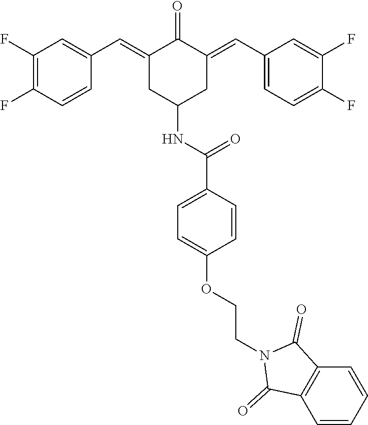 [SMILES: O=C1/C(=C/c2ccc(F)c(F)c2)CC(NC(=O)c2ccc(OCCN3C(=O)c4ccccc4C3=O)cc2)C/C1=C\c1ccc(F)c(F)c1]

### N-(3,5-Bis((E)-3,4-difluorobenzylidene)-4-oxocyclohexyl)-4-(2-(1,3-dioxoisoindolin-2-yl)ethoxy)benzamide (JCSO39)

¹H NMR (500 MHz, CDCl₃) δ 7.91-7.84 (m, 2H), 7.81 (s, 2H), 7.78-7.72 (m, 2H), 7.62 (d, J=8.3 Hz, 2H), 7.28-7.19 (m, 6H), 6.88 (d, J=8.4 Hz, 2H), 6.14 (d, J=7.3 Hz, 1H), 4.52-4.48 (m, 1H), 4.26 (t, J=5.8 Hz, 2H), 4.12 (t, J=5.8 Hz, 2H), 3.27 (bd, J=16.0 Hz, 2H), 3.07 (dd, J=16.0, 8.2 Hz, 2H). ¹³C NMR (126 MHz, CDCl₃) δ 187.9, 168.2, 166.5, 161.2, 150.7 (dd, J_{CF}=253.2 Hz, J_{C-CF}=12.6 Hz, 2C), 150.3 (dd, J_{CF}=250.7 Hz, J_{C-CF}=13.8 Hz, 2C), 137.9 (2C), 134.8 (2C), 132.8 (2C), 132.1 (dd, J_{C-C-CF}=6.3 Hz, J_{C-C-CF}=3.7 Hz, 2C), 132.0 (2C), 128.8 (2C), 127.2 (dd, J_{C-C-CF}=6.3 Hz, J_{C-C-CF}=3.7 Hz, 2C), 126.7, 123.5 (2C), 119.0 (d, J_{C-CF}=17.6 Hz, 2C), 117.7 (d, J_{C-CF}=16.3 Hz, 2C), 114.5 (2C), 64.9, 44.6, 37.1, 33.8. ¹⁹F NMR (376 MHz, CDCl₃) δ 131.1 (d, J_{FF}=30.0 Hz, 2F), 129.7 (d, J_{FF}=30.0 Hz, 2F). HR-APCI m/z calcd for C₃₇H₂₇F₄N₂O₅ [M+H]=655.1806, found 655.1860.

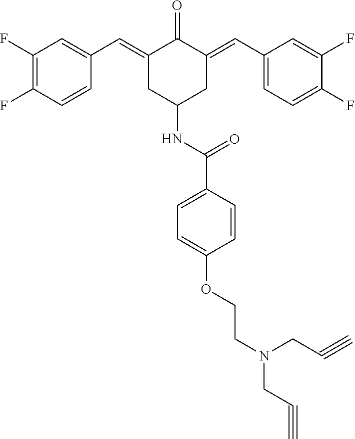 [SMILES: C#CCN(CC#C)CCOc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-(2-(di(prop-2-yn-1-yl)amino)ethoxy)-benzamide (JCSO40)

¹H NMR (500 MHz, CDCl₃) δ 7.78 (s, 2H), 7.63 (d, J=8.7 Hz, 2H), 7.25-7.14 (m, 6H), 6.88 (d, J=8.8 Hz, 2H), 6.16 (d, J=7.3 Hz, 1H), 4.53-4.45 (m, 1H), 4.12 (t, J=5.6 Hz, 2H), 3.55 (d, J=2.5 Hz, 4H), 3.25 (d, J=15 Hz, 2H), 3.15-2.96 (m, 4H), 2.26 (t, J=2.4 Hz, 2H). ¹³C NMR (126 MHz, CDCl₃) δ 187.9, 166.6, 161.5, 150.7 (dd, J_{CF}=254.5 Hz, J_{C-CF}=12.6 Hz, 2C), 150.3 (dd, J_{CF}=249.4 Hz, J_{C-CF}=13.8 Hz, 2C), 137.9 (2C), 132.8 (2C), 132.2 (dd, J_{C-C-CF}=6.3 Hz, J_{C-C-CF}=3.7 Hz, 2C), 128.8 (2C), 127.2 (dd, J_{C-C-CF}=6.3 Hz, J_{C-C-CF}=3.7 Hz, 2C), 126.4, 119.0 (d, J_{C-CF}=17.6 Hz, 2C), 117.7 (d, J_{C-CF}=16.3 Hz, 2C), 114.4 (2C), 78.5, 73.6, 66.7, 51.6, 44.6, 43.1, 33.8. ¹⁹F NMR (376 MHz, CDCl₃) δ −135.0 (d, J_{FF}=22.5 Hz, 2F), −136.5 (d, J_{FF}=18.8 Hz, 2F). HR-APCI m/z calcd for C₃₅H₂₉F₄N₂O₃ [M+H]=601.2108, found 601.2117.

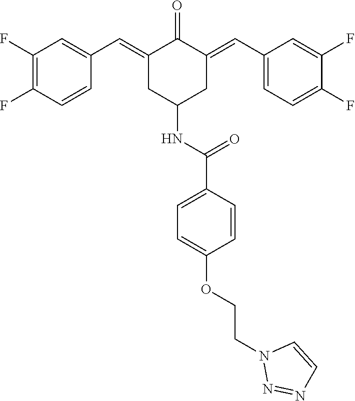 [SMILES: O=C1/C(=C/c2ccc(F)c(F)c2)CC(NC(=O)c2ccc(OCCn3ccnn3)cc2)C/C1=C\c1ccc(F)c(F)c1]

### 4-(2-(1H-1,2,3-Triazol-1-yl)ethoxy)-((E)-3,4-difluorobenzylidene)-4-oxocyclohexyl)benz-amide (JCS018)

¹H NMR (500 MHz, CDCl₃) δ 7.79 (s, 2H), 7.65-7.56 (m, 4H), 7.25-7.15 (m, 6H), 6.85 (d, J=8.4 Hz, 2H), 6.11 (d, J=7.1 Hz, 1H), 4.83 (t, J=5.5 Hz, 2H), 4.51-4.46 (m, 3H), 3.24 (bd, J=15.8 Hz, 2H), 3.05 (dd, J=15.8, 8.1 Hz, 2H). ¹³C NMR (126 MHz, CDCl₃) δ 187.9, 166.5, 161.0, 150.7 (dd, J_{CF}=253.2 Hz, J_{C-CF}=12.6 Hz, 2C), 150.3 (dd, J_{CF}=249.4 Hz, J_{C-CF}=13.8 Hz, 2C), 138.0 (2C), 134.7, 132.7 (2C), 132.2 (dd, J_{C-C-CF}=5.0 Hz, J_{C-C-CF}=2.5 Hz, 2C), 132.0, 128.8 (2C), 127.2 (dd, J_{C-C-CF}=6.3 Hz, J_{C-C-CF}=3.7 Hz, 2C), 126.9, 119.0 (d, J_{C-CF}=17.6 Hz, 2C), 117.7 (d, J_{C-CF}=17.6 Hz, 2C), 114.6 (2C), 66.1, 53.8, 44.6, 33.8. ¹⁹F NMR (376 MHz, CDCl₃) δ −135.0 (d, J_{FF}=22.5 Hz, 2F), −136.5 (d, J_{FF}=22.5 Hz, 2F). HR-APCI m/z calcd for C₃₁H₂₅F₄N₄O₃ [M−H]=577.1868, found 577.1868.

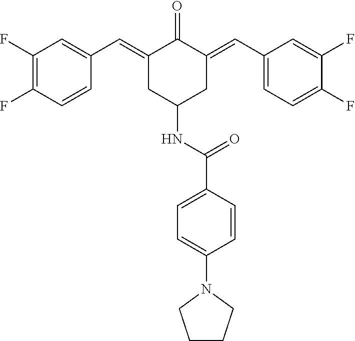 [SMILES: O=C1/C(=C/c2ccc(F)c(F)c2)CC(NC(=O)c2ccc(N3CCCC3)cc2)C/C1=C\c1ccc(F)c(F)c1]

### N-(3,5-Bis((E)-3,4-difluorobenzylidene)-4-oxocyclohexyl)-4-(pyrrolidin-1-yl)benzamide (JCS019)

¹H NMR (500 MHz, CDCl₃) δ 7.77 (s, 2H), 7.57 (d, J=8.6 Hz, 2H), 7.26-7.13 (m, 6H), 6.45 (d, J=8.5 Hz, 2H), 6.11 (d, J=7.3 Hz, 1H), 4.52-4.46 (m, 1H), 3.33-3.27 (m, 4H), 3.23 (bd, J=15.8 Hz, 2H), 3.04 (dd, J=15.7, 8.3 Hz, 2H), 2.03-1.99 (m, 4H). ¹³C NMR (126 MHz, CDCl₃) δ 188.2, 167.2, 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 150.2, 137.7 (2C), 133.1 (2C), 132.3 (dd, J_{C-C-CF}=5.0 Hz, J_{C-C-CF}=2.5 Hz, 2C), 128.7 (2C), 127.1 (dd, J_{C-C-CF}=6.3 Hz, J_{C-C-CF}=2.5 Hz, 2C), 119.7, 119.1 (d, J_{C-CF}=17.6 Hz, 2C), 117.7 (d, J_{C-CF}=17.6 Hz, 2C), 110.9 (2C), 47.6, 44.4, 34.0, 25.5. ¹⁹F NMR (376 MHz, CDCl₃) δ −135.2 (d, J_{FF}=18.8 Hz, 2F), −136.6 (d, J_{FF}=18.8 Hz, 2F). HR-APCI m/z calcd for C₃₁H₂₇F₄N₂O₂ [M+H]=535.2003, found 535.2011.

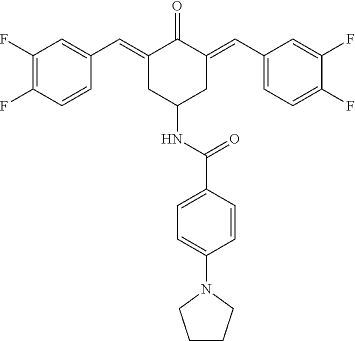 [SMILES: O=C1/C(=C/c2ccc(F)c(F)c2)CC(NC(=O)c2ccc(N3CCCC3)cc2)C/C1=C\c1ccc(F)c(F)c1]

### N-(3,5-Bis((E)-3,4-difluorobenzylidene)-4-oxocyclohexyl)-4-(diethylamino)benzamide (JCSO20)

¹H NMR (500 MHz, CDCl₃) δ 7.77 (s, 2H), 7.56 (d, J=8.4 Hz, 2H), 7.26-7.10 (m, 6H), 6.57 (d, J=8.2 Hz, 2H), 6.10 (d, J=6.4 Hz, 1H), 4.52-4.48 (m, 1H), 3.36 (q, J=6.9 Hz, 4H), 3.23 (bd, J=15.3 Hz, 2H), 3.05 (dd, J=15.6, 7.9 Hz, 2H), 1.15 (t, J=7.0 Hz, 6H). ¹³C NMR (126 MHz, CDCl₃) δ 188.1, 167.0, 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 150.2, 137.7 (2C), 133.1 (2C), 132.3 (dd, J_{C-C-CF}=5.0 Hz, J_{C-C-CF}=2.5 Hz, 2C), 128.7 (2C), 127.1 (dd, J_{C-C-CF}=5.0 Hz, J_{C-C-CF}=3.7 Hz, 2C), 119.5, 119.0 (d, J_{C-CF}=17.6 Hz, 2C), 117.7 (d, J_{C-CF}=17.6 Hz, 2C), 110.9 (2C), 44.5, 44.3, 33, 12.5. ¹⁹F NMR (376 MHz, CDCl₃) δ 130.9 (d, J_{FF}=26.3 Hz, 2F), 129.6 (d, J_{FF}=26.3 Hz, 2F). HR-APCI m/z calcd for C₃₁H₂₉F₄N₂O₂ [M+H]=537.2003, found 537.2011.

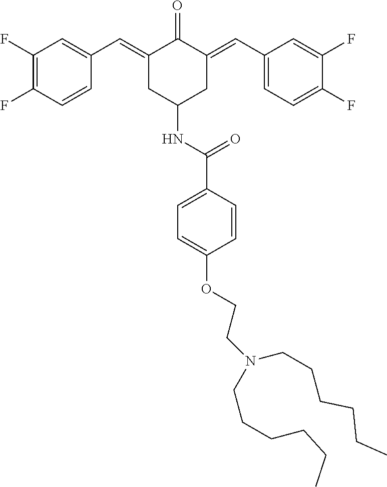 [SMILES: CCCCCCN(CCCCCC)CCOc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-(2-(dihexylamino)ethoxy)benzamide (JCSO21)

¹H NMR (500 MHz, CDCl₃) δ 7.77 (s, 2H), 7.63 (d, J=8.7 Hz, 2H), 7.26-7.13 (m, 6H), 6.86 (d, J=8.7 Hz, 2H), 6.24 (d, J=7.3 Hz, 1H), 4.50-4.45 (m, 1H), 4.03 (t, J=6.2 Hz, 2H), 3.24 (bd, J=15.8 Hz, 2H), 3.05 (dd, J=15.8, 8.4 Hz, 2H), 2.85 (t, J=6.2 Hz, 2H), 2.56-2.46 (m, 4H), 1.45 (p, J=7.0 Hz, 4H), 1.31-1.25 (m, 12H), 0.87 (t, J=6.6 Hz, 6H). ¹³C NMR (126 MHz, CDCl₃) δ 187.9, 166.7, 161.8, 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 137.8 (2C), 132.8 (2C), 132.2 (dd, J_{C-C-CF}=6.3 Hz, J_{C-C-CF}=3.7 Hz, 2C), 128.8 (2C), 127.1 (dd, J_{C-C-CF}=6.3 Hz, J_{C-C-CF}=3.7 Hz, 2C), 126.1, 119.0 (d, J_{C-CF}=17.6 Hz, 2C), 117.7 (d, J_{C-CF}=17.6 Hz, 2C), 114.4 (2C), 66.9, 55.1, 52.7, 44.6, 33.8, 31.9, 27.2, 27.1, 22.7, 14.1. ¹⁹F NMR (376 MHz, CDCl₃) δ −130.9 (d, J_{FF}=26.3 Hz, 2F), −129.6 (d, J_{FF}=26.3 Hz, 2F). HR-APCI m/z calcd for C₄₁H₄₉F₄N₂O₃ [M+H]=693.3673, found 693.3685.

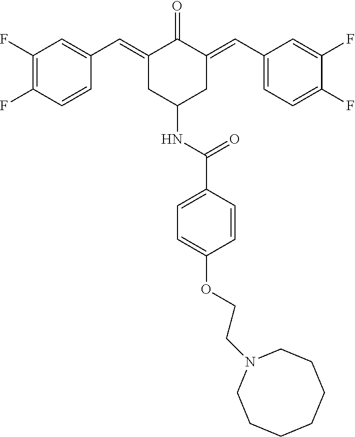 [SMILES: O=C1/C(=C/c2ccc(F)c(F)c2)CC(NC(=O)c2ccc(OCCN3CCCCCCC3)cc2)C/C1=C\c1ccc(F)c(F)c1]

### 4-(2-(Azocan-1-yl)ethoxy)-N-(3,5-bis((E)-3,4-difluorobenzylidene)-4-oxocyclohexyl)benzamide (JCSO22)

¹H NMR (500 MHz, CDCl₃) δ 7.77 (s, 2H), 7.64 (d, J=8.9 Hz, 2H), 7.25-7.14 (m, 6H), 6.87 (d, J=8.6 Hz, 2H), 6.26 (d, J=6.5 Hz, 1H), 4.50-4.46 (m, 1H), 4.05 (t, J=6.1 Hz, 2H), 3.24 (bd, J=15.8 Hz, 2H), 3.05 (dd, J=15.8, 8.4 Hz, 2H), 2.91 (t, J=6.1 Hz, 2H), 2.67 (t, J=4.8 Hz, 4H), 1.64-1.53 (m, 10H). ¹³C NMR (126 MHz, CDCl₃) δ 187.9, 166.7, 161.9, 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 137.8 (2C), 132.8 (2C), 132.2 (dd, J_{C-C-CF}=6.3 Hz, J_{C-C-CF}=3.7 Hz, 2C), 128.8 (2C), 127.1 (dd, J_{C-C-CF}=6.3 Hz, J_{C-C-CF}=3.7 Hz, 2C), 126.1, 119.0 (d, J_{C-CF}=17.6 Hz, 2C), 117.7 (d, J_{C-CF}=17.6 Hz, 2C), 114.4 (2C), 67.0, 57.2, 54.3, 44.6, 33.8, 27.9, 27.3, 26.3. ¹⁹F NMR (376 MHz, CDCl₃) δ −135.0 (d, J_{FF}=18.8 Hz, 2F), −136.5 (d, J_{FF}=18.8 Hz, 2F). HR-APCI m/z calcd for C₃₆H₃₇F₄N₂O₃ [M+H]=621.2734, found 621.2739.

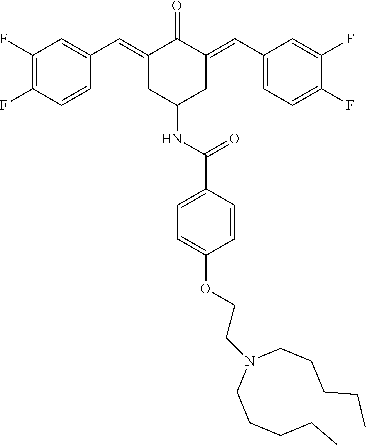 [SMILES: CCCCCN(CCCCC)CCOc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-(2-(dipentylamino)ethoxy)benzamide (JCS023)

¹H NMR (500 MHz, CDCl₃) δ 7.81 (s, 2H), 7.63 (d, J=8.8 Hz, 2H), 7.30-7.26 (m, 1H), 7.25-7.16 (m, 5H), 6.87 (d, J=8.8 Hz, 2H), 6.09 (d, J=7.3 Hz, 1H), 4.53-4.47 (m, 1H), 4.08 (t, J=6.0 Hz, 2H), 3.26 (bd, J=15.5 Hz, 2H), 3.07 (dd, J=15.5, 8.0 Hz, 2H), 2.93 (t, J=6.0 Hz, 2H), 2.57-2.53 (m, 4H), 1.48 (p, J=7.3 Hz, 4H), 1.35-1.21 (m, 8H), 0.88 (t, J=7.0 Hz, 6H). ¹³C NMR (126 MHz, CDCl₃) δ 188.0, 166.6, 161.7, 150.8 (dd, J_{CF}=253.2 Hz, J_{C-CF}=12.6 Hz, 2C), 150.3 (dd, J_{CF}=249.4 Hz, J_{C-CF}=12.6 Hz, 2C), 138.0 (2C), 132.8 (2C), 132.2 (dd, J_{C-C-CF}=6.3 Hz, J_{C-C-CF}=3.7 Hz, 2C), 128.8 (2C), 127.1 (dd, J_{C-C-CF}=6.3 Hz, J_{C-C-CF}=3.7 Hz, 2C), 126.2, 119.0 (d, J_{C-CF}=17.6 Hz, 2C), 117.7 (d, J_{C-CF}=17.6 Hz, 2C), 114.4 (2C), 66.7, 54.9, 52.7, 44.6, 33.8, 29.7, 26.5, 22.7, 14.2. ¹⁹F NMR (376 MHz, CDCl₃) δ −135.0 (d, J_{FF}=22.5 Hz, 2F), −136.5 (d, J_{FF}=22.5 Hz, 2F). HR-APCI m/z calcd for C₃₉H₄₅F₄N₂O₃ [M+H]=665.3360, found 665.3377.

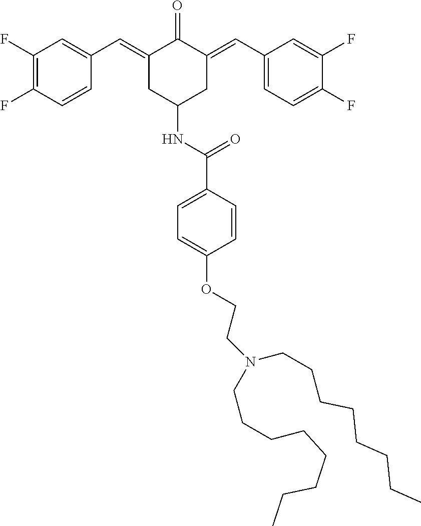 [SMILES: CCCCCCCCN(CCCCCCCC)CCOc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-(2-(dioctylamino)ethoxy)benzamide (JCS024)

¹H NMR (500 MHz, CDCl₃) δ 7.79 (s, 2H), 7.63 (d, J=8.8 Hz, 2H), 7.26-7.16 (m, 6H), 6.87 (d, J=8.8 Hz, 2H), 6.14 (d, J=7.3 Hz, 1H), 4.52-4.46 (m, 1H), 4.04 (t, J=6.2 Hz, 2H), 3.25 (bd, J=15.8 Hz, 2H), 3.06 (dd, J=15.8, 8.3 Hz, 2H), 2.86 (t, J=6.2 Hz, 2H), 2.55-2.43 (m, 4H), 1.48-1.42 (m, 4H), 1.30-1.25 (m, 20H), 0.87 (t, J=6.9 Hz, 6H). ¹³C NMR (126 MHz, CDCl₃) δ 187.9, 166.7, 161.8, 150.7 (dd, J_{CF}=253.2 Hz, J_{C-CF}=12.6 Hz, 2C), 150.3 (dd, J_{CF}=250.7 Hz, J_{C-CF}=12.6 Hz, 2C), 137.9 (2C), 132.8 (2C), 132.2 (dd, J_{C-C-CF}=6.3 Hz, J_{C-C-CF}=3.7 Hz, 2C), 128.8 (2C), 127.1 (dd, J_{C-C-CF}=6.3 Hz, J_{C-C-CF}=3.7 Hz, 2C), 126.1, 119.0 (d, J_{C-CF}=17.6 Hz, 2C), 117.7 (d, J_{C-CF}=17.6 Hz, 2C), 114.4 (2C), 66.9, 55.1, 52.8, 44.6, 33.8, 31.9, 29.6, 29.4, 27.6, 27.1, 22.7, 14.2. ¹⁹F NMR (376 MHz, CDCl₃) δ −135.0 (d, J_{FF}=22.5 Hz, 2F), −136.5 (d, J_{FF}=18.8 Hz, 2F). HR-APCI m/z calcd for C₄₅H₅₇F₄N₂O₃ [M+H]=749.4299, found 749.4295.

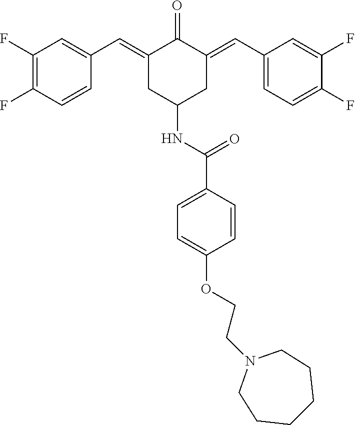 [SMILES: O=C1/C(=C/c2ccc(F)c(F)c2)CC(NC(=O)c2ccc(OCCN3CCCCCC3)cc2)C/C1=C\c1ccc(F)c(F)c1]

### 4-(2-(Azepan-1-yl)ethoxy)-N-(3,5-bis((E)-3,4-difluorobenzylidene)-4-oxocyclohexyl)benzamide (JCSO26)

¹H NMR (500 MHz, CDCl₃) δ 7.78 (s, 2H), 7.63 (d, J=8.7 Hz, 2H), 7.27-7.09 (m, 6H), 6.87 (d, J=8.7 Hz, 2H), 6.20 (d, J=7.3 Hz, 1H), 4.52-4.45 (m, 1H), 4.08 (t, J=6.1 Hz, 2H), 3.24 (bd, J=15.8 Hz, 2H), 3.05 (dd, J=15.9, 8.2 Hz, 2H), 2.95 (t, J=6.1 Hz, 2H), 2.84-2.71 (m, 4H), 1.76-1.54 (m, 8H).

¹³C NMR (126 MHz, CDCl₃) δ 187.9, 166.7, 161.8, 150.7 (dd, J_{CF}=254.5 Hz, J_{C-CF}=12.6 Hz, 2C), 150.2 (dd, J_{CF}=250.7 Hz, J_{C-CF}=12.6 Hz, 2C), 137.9 (2C), 132.8 (2C), 132.2 (dd, J_{C-C-CF}=6.3 Hz, J_{C-C-CF}=3.7 Hz, 2C), 128.8 (2C), 127.1 (dd, J_{C-C-CF}=6.3 Hz, J_{C-C-CF}=3.7 Hz, 2C), 126.2, 119.0 (d, J_{C-CF}=17.6 Hz, 2C), 117.7 (d, J_{C-CF}=17.6 Hz, 2C), 114.5 (2C), 66.7, 56.1, 55.9, 44.6, 33.8, 27.8, 27.1. ¹⁹F NMR (376 MHz, CDCl₃) δ −135.0 (d, J_{FF}=18.8 Hz, 2F), −136.5 (d, J_{FF}=22.6 Hz, 2F). HR-APCI m/z calcd for C₃₅H₃₅F₄N₂O₃ [M+H]=607.2578, found 607.2575.

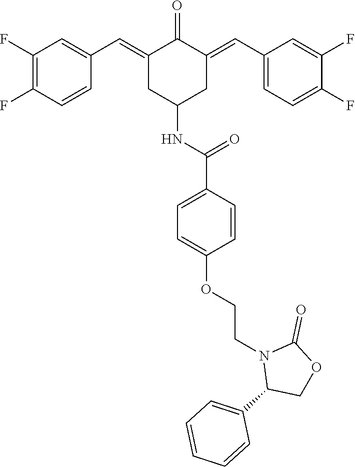 [SMILES: O=C1/C(=C/c2ccc(F)c(F)c2)CC(NC(=O)c2ccc(OCCN3C(=O)OC[C@@H]3c3ccccc3)cc2)C/C1=C\c1ccc(F)c(F)c1]

### N-(3,5-Bis((E)-3,4-difluorobenzylidene)-4-oxocyclohexyl)-4-(2-((S)-2-oxo-4-phenyloxazolidin-3-yl)ethoxy)benzamide (JCSO45)

¹H NMR (500 MHz, CDCl₃) δ 7.75 (s, 2H), 7.69 (d, J=8.8 Hz, 2H), 7.43-7.34 (m, 3H), 7.34-7.28 (m, 2H), 7.27-7.20 (m, 2H), 7.18-7.15 (m, 4H), 6.78 (d, J=8.8 Hz, 2H), 6.67 (d, J=7.2 Hz, 1H), 4.96 (dd, J=8.9, 7.0 Hz, 1H), 4.61 (t, J=8.8 Hz, 1H), 4.48-4.41 (m, 1H), 4.13 (dd, J=8.8, 7.0 Hz, 1H), 4.09-4.05 (m, 1H), 3.96-3.93 (m, 1H), 3.76-3.72 (m, 1H), 3.26 (dd, J=15.1, 3.9 Hz, 2H), 3.20 (m, 1H), 3.08-3.01 (m, 2H). ¹³C NMR (126 MHz, CDCl₃) δ 187.9, 166.5, 160.9, 158.4, 150.6 (dd, J_{CF}=253.2 Hz, J_{C-CF}=12.6 Hz, 2C), 150.2 (dd, J_{CF}=249.4 Hz, J_{C-CF}=12.6 Hz, 2C), 137.6 (2C), 137.5 (2C), 133.1 (2C), 132.2 (dd, J_{C-C-CF}=6.3 Hz, J_{C-C-CF}=3.7 Hz, 2C), 129.5 (2C), 129.4 (2C), 129.0 (2C), 127.3 (dd, J_{C-C-CF}=6.3 Hz, J_{C-C-CF}=3.7 Hz, 2C), 127.2, 126.2, 118.9 (d, J_{C-CF}=17.6 Hz, 2C), 117.7 (d, J_{C-CF}=17.6 Hz, 2C), 114.1 (2C), 70.2, 65.8, 61.0, 44.9, 41.5, 33.8. ¹⁹F NMR (376 MHz, CDCl₃) δ 131.0 (d, J_{FF}=18.8 Hz, 2F), 129.6 (d, J_{FF}=22.6 Hz, 2F). HR-APCI m/z calcd for C₃₈H₃₁F₄N₂O₅ [M+H]=671.2163, found 671.2236.

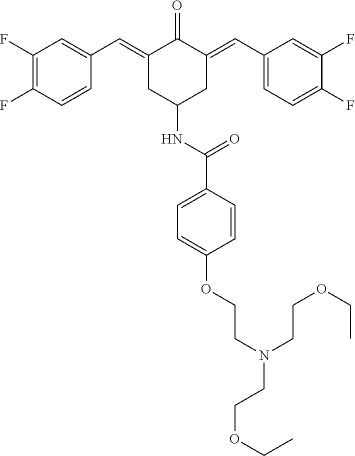 [SMILES: CCOCCN(CCOCC)CCOc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-(2-(bis(2-ethoxyethyl)amino)ethoxy)-benzamide (JCSO49)

¹H NMR (500 MHz, CDCl₃) δ 7.80 (s, 2H), 7.62 (d, J=8.8 Hz, 2H), 7.28-7.16 (m, 6H), 6.88 (d, J=8.8 Hz, 2H), 6.09 (d, J=7.3 Hz, 1H), 4.53-4.47 (m, 1H), 4.06 (t, J=6.1 Hz, 2H), 3.58-3.41 (m, 8H), 3.25 (bd, J=15.2 Hz, 2H), 3.14-3.01 (m, 4H), 2.84 (t, J=6.1 Hz, 4H), 1.17 (t, J=7.0 Hz, 6H). ¹³C NMR (126 MHz, CDCl₃) δ 188.0, 166.7, 161.9, 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 137.9 (2C), 132.8 (2C), 132.2 (dd, J_{C-C-CF}=6.3 Hz, J_{C-C-CF}=3.7 Hz, 2C), 128.7 (2C), 127.1 (dd, J_{C-C-CF}=6.3 Hz, J_{C-C-CF}=3.7 Hz, 2C), 126.1, 119.0 (d, J_{C-CF}=17.6 Hz, 2C), 117.7 (d, J_{C-CF}=17.6 Hz, 2C), 114.4 (2C), 69.2, 67.0, 66.6, 55.0, 54.1, 44.6, 33.8, 15.3. ¹⁹F NMR (376 MHz, CDCl₃) δ 131.1 (d, J_{FF}=11.2 Hz, 2F), 129.7 (d, J_{FF}=30.0 Hz, 2F). HR-APCI m/z calcd for C₃₇H₄₁F₄N₂O₅ [M+H]=669.2946, found 669.2922.

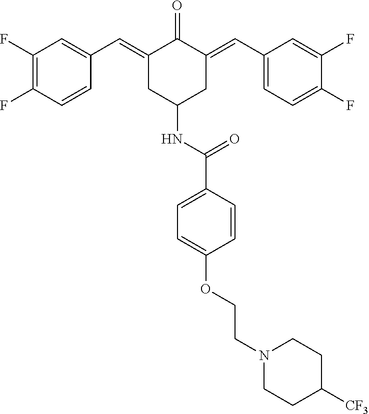 [SMILES: O=C1/C(=C/c2ccc(F)c(F)c2)CC(NC(=O)c2ccc(OCCN3CCC(C(F)(F)F)CC3)cc2)C/C1=C\c1ccc(F)c(F)c1]

### N-(3,5-bis((E)-3,4-difluorobenzylidene)-4-oxocyclohexyl)-4-(2-(4-(trifluoromethyl)piperidin-1-yl)ethoxy)benzamide (JCS077)

¹H NMR (500 MHz, CDCl₃) δ 7.79 (s, 2H), 7.63 (d, J=8.4 Hz, 2H), 7.26-7.14 (m, 6H), 6.87 (d, J=8.5 Hz, 2H), 6.12 (d, J=7.3 Hz, 1H), 4.52-4.46 (m, 1H), 4.11 (t, J=5.8 Hz, 2H), 3.25 (bd, J=15.0 Hz, 2H), 3.08-3.03 (m, 4H), 2.81 (t, J=5.7 Hz, 2H), 2.11 (t, J=11.3 Hz, 2H), 2.05-2.16 (m, 1H), 1.86-1.83 (m, 2H), 1.69-1.61 (m, 2H). ¹³C NMR (126 MHz, CDCl₃) δ 187.9, 166.6, 161.6, 150.7 (dd, J_{CF}=253.4 Hz, J_{C-CF}=13.8 Hz, 2C), 150.3 (dd, J_{CF}=249.4 Hz, J_{C-CF}=12.6 Hz, 2C), 137.9 (2C), 132.8 (2C), 132.8, 132.2 (dd, J_{C-C-CF}=6.3 Hz, J_{C-C-CF}=5.0 Hz, 2C), 128.8, 127.2 (dd, J_{C-C-CF}=6.3 Hz, J_{C-C-CF}=3.8 Hz, 2C), 126.4, 119.0 (d, J_{C-CF}=17.6 Hz, 2C), 117.7 (d, J_{C-F}=17.6 Hz, 2C), 114.5 (2C), 66.3, 57.1, 53.0, 44.6, 40.2 (q, J_{C-F}=27.7 Hz, 1C), 38.7, 33.8, 24.6. ¹⁹F NMR (376 MHz, CDCl₃) δ 131.1 (d, J_{FF}=26.6 Hz, 2F), 129.7 (d, J_{FF}=26.6 Hz, 2F), −73.6 (s, 3F). HR-APCI m/z calcd for C₃₅H₃₂F₇N₂O₃ [M+H]=661.2269, found 661.2295.

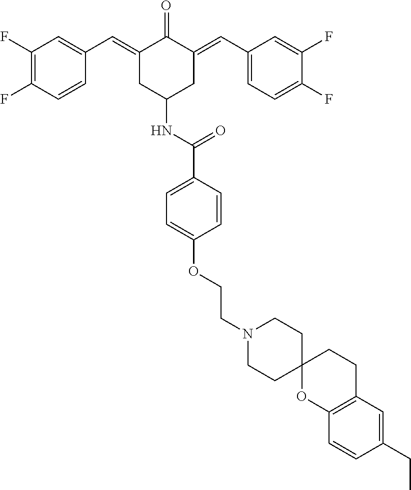 [SMILES: CCc1ccc2c(c1)CCC1(CCN(CCOc3ccc(C(=O)NC4C/C(=C\c5ccc(F)c(F)c5)C(=O)/C(=C/c5ccc(F)c(F)c5)C4)cc3)CC1)O2]

### N-(3,5-bis((E)-3,4-difluorobenzylidene)-4-oxocyclohexyl)-4-(2-(6-ethylspiro[chromane-2,4′-piperidin]-1′-yl)ethoxy)benzamide (JCS078)

¹H NMR (500 MHz, CDCl₃) δ 7.79 (s, 2H), 7.63 (d, J=8.2 Hz, 2H), 7.29-7.13 (m, 6H), 6.97-6.83 (m, 4H), 6.75 (d, J=8.3 Hz, 1H), 6.15 (d, J=7.3 Hz, 1H), 4.56-4.42 (m, 1H), 4.13 (t, J=5.8 Hz, 2H), 3.25 (bd, J=15.1 Hz, 2H), 3.05 (dd, J=14.9, 7.9 Hz, 2H), 2.85 (t, J=5.8 Hz, 2H), 2.74 (t, J=6.9 Hz, 4H), 2.60-2.52 (m, 4H), 1.85-1.76 (m, 4H), 1.66 (t, J=10.5 Hz, 2H), 1.19 (t, J=7.4 Hz, 3H). ¹³C NMR (126 MHz, CDCl₃) δ 187.8, 166.6, 161.6, 151.1, 150.6 (dd, J_{CF}=253.2 Hz, J_{C-CF}=13.8 Hz, 2C), 150.1 (dd, J_{CF}=248.2 Hz, J_{C-CF}=12.6 Hz, 2C), 137.7 (2C), 135.6, 132.7 (2C), 132.1 (dd, J_{C-C-CF}=6.3 Hz, J_{C-C-CF}=5.0 Hz, 2C), 128.7 (2C), 128.6, 127.1 (dd, J_{C-C-CF}=6.3 Hz, J_{C-C-CF}=3.8 Hz, 2C), 126.7, 126.1, 121.0, 118.9 (d, J_{C-CF}=17.6 Hz, 2C), 117.6 (d, J_{C-CF}=17.6 Hz, 2C), 117.0, 114.4 (2C), 71.8, 66.3, 57.1, 49.7, 44.6, 34.4, 32.1, 27.9, 21.6, 15.8. ¹⁹F NMR (376 MHz, CDCl₃) δ 131.2 (d, J_{FF}=26.6 Hz, 2F), 129.7 (d, J_{FF}=26.6 Hz, 2F). HR-APCI m/z calcd for C₄₄H₄₃F₄N₂O₄ [M+H]=739.3126, found 739.3153.

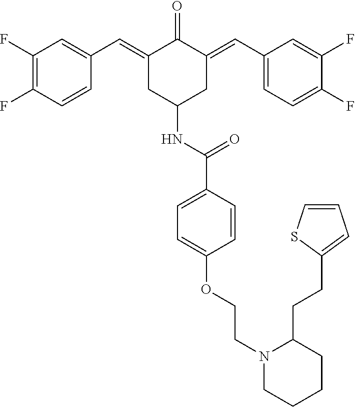 [SMILES: O=C1/C(=C/c2ccc(F)c(F)c2)CC(NC(=O)c2ccc(OCCN3CCCCC3CCc3cccs3)cc2)C/C1=C\c1ccc(F)c(F)c1]

### N-(3,5-bis((E)-3,4-difluorobenzylidene)-4-oxocyclohexyl)-4-(2-(2-(2-(thiophen-2-yl)ethyl)piperidin-1-yl)ethoxy)benzamide (JCS079)

¹H NMR (500 MHz, CDCl₃) δ 7.77 (s, 2H), 7.62 (d, J=8.8 Hz, 2H), 7.25-7.13 (m, 6H), 7.09 (dd, J=5.1, 1.2 Hz, 1H), 6.89 (dd, J=5.2, 3.4 Hz, 1H), 6.84 (d, J=8.8 Hz, 2H), 6.77 (d, J=3.2 Hz, 1H), 6.24 (d, J=7.4 Hz, 1H), 4.48-4.40 (m, 1H), 4.05 (t, J=6.0 Hz, 2H), 3.24 (bd, J=15.0 Hz, 2H), 3.13-3.00 (m, 3H), 2.96-2.90 (m, 2H), 2.84-2.78 (m, 2H), 2.53-2.39 (m, 2H), 2.06-1.96 (m, 1H), 1.87-1.81 (m, 1H), 1.73-1.68 (m, 2H), 1.58-1.53 (m, 2H), 1.46-1.32 (m, 2H). ¹³C NMR (126 MHz, CDCl₃) δ 187.9, 166.7, 160.9, 150.6 (dd, J_{CF}=265.8 Hz, J_{C-CF}=12.6 Hz, 2C), 150.1 (dd, J_{CF}=249.4 Hz, J_{C-CF}=12.6 Hz, 2C), 144.0, 137.5 (2C), 133.1 (2C), 132.3 (dd, J_{C-C-CF}=6.3 Hz, J_{C-C-CF}=3.7 Hz, 2C), 129.0, 127.2 (dd, J_{C-C-CF}=6.3 Hz, J_{C-C-CF}=3.7 Hz, 2C), 127.0, 126.8, 124.6, 123.5, 119.0 (d, J_{C-CF}=17.6 Hz, 2C), 117.7 (d, J_{C-CF}=17.6 Hz, 2C), 114.3, 64.7, 61.1, 52.4, 51.2, 45.0, 33.8, 32.4, 28.0, 26.0, 23.2, 21.9. ¹⁹F NMR (376 MHz, CDCl₃) δ −135.2 (d, J_{FF}=22.6 Hz, 2F), −136.6 (d, J_{FF}=22.6 Hz, 2F). HR-APCI m/z calcd for C₄₀H₃₉F₄N203S [M+H]=703.2617, found 703.2612.

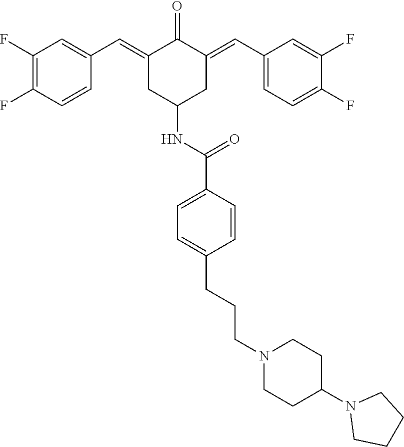 [SMILES: O=C1/C(=C/c2ccc(F)c(F)c2)CC(NC(=O)c2ccc(CCCN3CCC(N4CCCC4)CC3)cc2)C/C1=C\c1ccc(F)c(F)c1]

### N-(3,5-bis((E)-3,4-difluorobenzylidene)-4-oxocyclohexyl)-4-(2-(4-(pyrrolidin-1-yl)piperidin-1-yl)ethoxy)benzamide (JCS080)

¹H NMR (500 MHz, CDCl₃) δ 7.78 (s, 2H), 7.63 (d, J=8.4 Hz, 2H), 7.26-7.16 (m, 6H), 6.87 (d, J=8.4 Hz, 2H), 6.19 (d, J=7.3 Hz, 1H), 4.51-4.45 (m, 1H), 4.09 (t, J=5.9 Hz, 2H), 3.25 (bd, J=15.0 Hz, 2H), 3.05 (dd, J=15.7, 7.9 Hz, 2H), 2.97-2.94 (m, 2H), 2.76 (t, J=5.9 Hz, 2H), 2.60 (t, J=5.8 Hz, 4H), 2.13 (t, J=11.4 Hz, 2H), 2.08-2.03 (m, 1H), 1.88-1.85 (m, 2H), 1.80-1.78 (m, 4H), 1.62-1.56 (m, 2H). ¹³C NMR (126 MHz, CDCl₃) δ 187.8, 166.5, 161.6, 150.6 (dd, J_{CF}=253.2 Hz, J_{C-CF}=13.8 Hz, 2C), 150.2 (dd, J_{CF}=249.4 Hz, J_{C-CF}=12.6 Hz, 2C), 137.7 (2C), 132.7 (2C), 132.1 (dd, J_{C-C-CF}=6.3 Hz, J_{C-C-CF}=5.0 Hz, 2C), 128.7 (2C), 127.0 (dd, J_{C-C-CF}=6.3 Hz, J_{C-C-CF}=3.8 Hz, 2C), 126.1, 118.9 (d, J_{C-CF}=17.6 Hz, 2C), 117.6 (d, J_{C-CF}=17.6 Hz, 2C), 114.4 (2C), 66.4, 61.6, 56.9, 53.0, 51.3, 44.5, 33.7, 29.7, 23.2. ¹⁹F NMR (376 MHz, CDCl₃) δ −135.2 (d, J_{FF}=18.8 Hz, 2F), −136.6 (d, J_{FF}=22.5 Hz, 2F). HR-APCI m/z calcd for C₃₈H₄₀F₄N₃O₃ [M+H]=662.2976, found 662.3000.

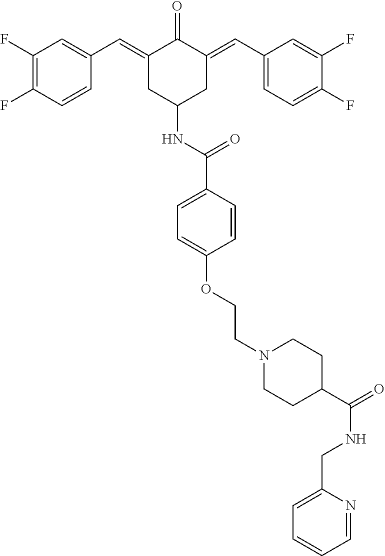 [SMILES: O=C1/C(=C/c2ccc(F)c(F)c2)CC(NC(=O)c2ccc(OCCN3CCC(C(=O)NCc4ccccn4)CC3)cc2)C/C1=C\c1ccc(F)c(F)c1]

### 1-(2-(4-((3,5-bis((E)-3,4-difluorobenzylidene)-4-oxocyclohexyl)carbamoyl)phenoxy)ethyl)-N-(pyridin-2-ylmethyl)piperidine-4-carboxamide (JCS081)

¹H NMR (500 MHz, CDCl₃) δ 8.51 (d, J=4.8 Hz, 1H), 7.78 (s, 2H), 7.66-7.62 (m, 3H), 7.29-7.14 (m, 8H), 6.92-6.86 (m, 3H), 6.25 (d, J=7.3 Hz, 1H), 4.53 (d, J=4.8 Hz, 2H), 4.52-4.41 (m, 1H), 4.12 (t, J=5.7 Hz, 2H), 3.25 (bd, J=15.0 Hz, 2H), 3.13-2.97 (m, 4H), 2.81 (t, J=5.8 Hz, 2H), 2.31-2.12 (m, 3H), 2.00-1.76 (m, 4H). ¹³C NMR (126 MHz, CDCl₃) δ 188.0, 174.8, 166.6, 161.6, 156.2, 150.7 (dd, J_{CF}=253.2 Hz, J_{C-CF}=13.8 Hz, 2C), 150.3 (dd, J_{CF}=243.1 Hz, J_{C-CF}=12.6 Hz, 2C), 149.0, 137.8 (2C), 136.92, 132.9 (2C), 132.2 (dd, J_{C-C-CF}=6.3 Hz, J_{C-C-CF}=5.0 Hz, 2C), 128.8 (2C), 127.2 (dd, J_{C-C-CF}=6.3 Hz, J_{C-C-CF}=3.8 Hz, 2C), 126.4, 122.5, 122.2, 119.0 (d, J_{C-CF}=17.6 Hz, 2C), 117.7 (d, J_{C-CF}=17.6 Hz, 2C), 114.5 (2C), 66.2, 57.2, 53.7, 44.7, 44.3, 42.9, 33.8, 28.8. ¹⁹F NMR (376 MHz, CDCl₃) δ −135.1 (d, J_{FF}=18.8 Hz, 2F), −136.5 (d, J_{FF}=22.5 Hz, 2F). HR-APCI m/z calcd for C₄₁H₃₉F₄N₄O₄ [M+H]=728.2936, found 728.2901.

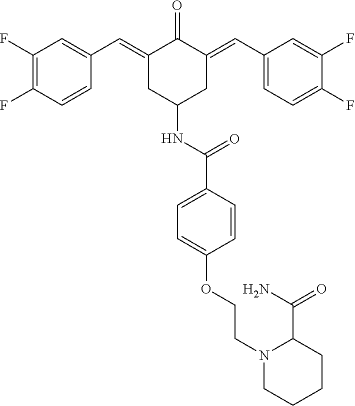 [SMILES: NC(=O)C1CCCCN1CCOc1ccc(C(=O)NC2C/C(=C\c3ccc(F)c(F)c3)C(=O)/C(=C/c3ccc(F)c(F)c3)C2)cc1]

### 1-(2-(4-((3,5-bis((E)-3,4-difluorobenzylidene)-4-oxocyclohexyl)carbamoyl)phenoxy)ethyl)piperidine-2-carboxamide (JCS083)

¹H NMR (500 MHz, CDCl₃) δ 7.75 (s, 2H), 7.64 (d, J=8.8 Hz, 2H), 7.25-7.15 (m, 6H), 6.81 (d, J=8.9 Hz, 2H), 6.53 (d, J=7.3 Hz, 1H), 5.74 (s, 2H), 4.47-4.42 (m, 1H), 4.15-4.12 (m, 2H), 3.25 (bd, J=15.0 Hz, 2H), 3.16 (t, J=5.0 Hz, 1H), 3.05 (dd, J=15.0, 8.4 Hz, 2H), 2.92-2.56 (m, 2H), 2.16-1.96 (m, 2H), 1.79-1.60 (m, 4H), 1.36-1.20 (m, 2H). ¹³C NMR (126 MHz, CDCl₃) δ 188.0, 166.6, 151.7, 150.6 (dd, J_{CF}=240.6 Hz, J_{C-CF}=13.8 Hz, 2C), 150.2 (dd, J_{CF}=249.4 Hz, J_{C-CF}=12.6 Hz, 2C), 137.7 (2C), 133.0 (2C), 133.00, 132.2 (dd, J_{C-C-CF}=6.3 Hz, J_{C-C-CF}=5.0 Hz, 2C), 129.0 (2C), 127.2 (dd, J_{C-C-CF}=6.3 Hz, J_{C-C-CF}=3.8 Hz, 2C), 126.7, 118.9 (d, J_{C-CF}=17.6 Hz, 2C), 117.7 (d, J_{C-CF}=17.6 Hz, 2C), 114.2 (2C), 68.0, 65.4, 55.5, 52.6, 44.8, 33.8, 30.4, 24.8, 23.3. ¹⁹F NMR (376 MHz, CDCl₃) δ −135.1 (d, J_{FF}=18.8 Hz, 2F), −136.5 (d, J_{FF}=22.5 Hz, 2F). HR-APCI m/z calcd for C₃₅H₃₄F₄N₃O₄ [M+H]=636.2558, found 636.2479.

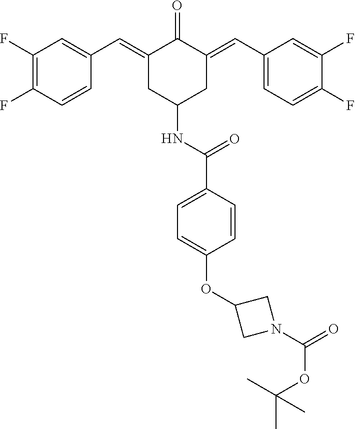 [SMILES: CC(C)(C)OC(=O)N1CC(Oc2ccc(C(=O)NC3C/C(=C\c4ccc(F)c(F)c4)C(=O)/C(=C/c4ccc(F)c(F)c4)C3)cc2)C1]

### tert-butyl3-(4-((3,5-bis((E)-3,4-difluorobenzylidene)-4-oxocyclohexyl)carbamoyl)phenoxy)azetidine-1-carboxylate (JCS084)

¹H NMR (500 MHz, CDCl₃) δ 7.78 (s, 2H), 7.63 (d, J=8.3 Hz, 2H), 7.31-7.09 (m, 6H), 6.71 (d, J=8.3 Hz, 2H), 6.21 (d, J=7.1 Hz, 1H), 4.88-4.86 (m, 1H), 4.53-4.42 (m, 1H), 4.28 (d, J=5.2 Hz, 2H), 3.96 (d, J=5.8 Hz, 2H), 3.24 (bd, J=15.1 Hz, 2H), 3.05 (dd, J=14.0, 7.9 Hz, 2H), 1.43 (s, 9H). ¹³C NMR (126 MHz, CDCl₃) δ 187.9, 166.4, 159.4, 156.1, 150.7 (dd, J_{CF}=254.5 Hz, J_{C-CF}=13.8 Hz, 2C), 150.3 (dd, J_{CF}=249.4 Hz, J_{C-CF}=12.6 Hz, 2C), 137.9 (2C), 132.7 (2C), 132.2 (dd, J_{C-C-CF}=6.3 Hz, J_{C-C-CF}=5.0 Hz, 2C), 129.1 (2C), 127.2 (dd, J_{C-C-CF}=6.3 Hz, J_{C-C-CF}=3.8 Hz, 2C), 127.1, 119.0 (d, J_{C-CF}=17.6 Hz, 2C), 117.7 (d, J_{C-CF}=17.6 Hz, 2C), 114.5 (2C), 80.1, 66.0, 56.2, 44.7, 33.8, 28.4. ¹⁹F NMR (376 MHz, CDCl₃) δ −135.1 (d, J_{FF}=18.8 Hz, 2F), −136.5 (d, J_{FF}=22.5 Hz, 2F). HR-APCI m/z calcd for C₃₅H₃₃F₄N₂O₅ [M+H]=637.2263, found 637.2320.

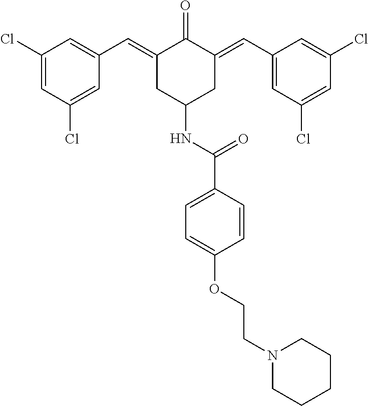 [SMILES: O=C1/C(=C/c2cc(Cl)cc(Cl)c2)CC(NC(=O)c2ccc(OCCN3CCCCC3)cc2)C/C1=C\c1cc(Cl)cc(Cl)c1]

### N-(3,5-Bis((E)-3,5-dichlorobenzylidene)-4-oxocyclohexyl)-4-(2-(piperidin-1-yl)ethoxy)benzamide (JCS061)

¹H NMR (500 MHz, CDCl₃) δ 7.68 (s, 2H), 7.64 (d, J=8.8 Hz, 2H), 7.34-7.31 (m, 2H), 7.23-7.21 (m, 4H), 6.87 (d, J=8.8 Hz, 2H), 6.27 (d, J=7.3 Hz, 1H), 4.54-4.46 (m, 1H), 4.10 (t, J=6.0 Hz, 2H), 3.20 (bd, J=16.2 Hz, 2H), 3.06 (dd, J=16.4, 8.4 Hz, 2H), 2.75 (t, J=6.0 Hz, 2H), 2.48 (t, J=5.3 Hz, 4H), 1.59 (p, J=5.6 Hz, 4H), 1.46-1.40 (m, 2H). ¹³C NMR (126 MHz, CDCl₃) δ 187.6, 166.7, 161.8, 137.9 (2C), 137.4 (2C), 135.3, 134.2 (2C), 129.1 (2C), 128.8 (2C), 128.2, 126.1 (2C), 114.5, 66.3, 57.8, 55.2, 44.4, 33.7, 26.0, 24.2. HR-APCI m/z calcd for C₃₄H₃₃C₁₄N₂O₃ [M+H]=657.1239, found 657.1222.

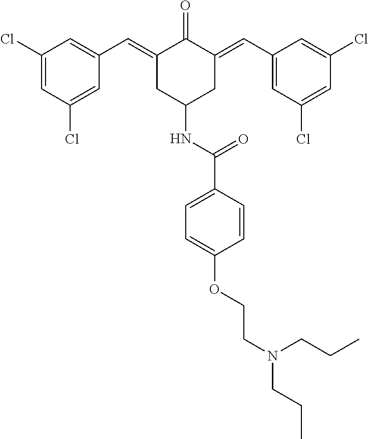 [SMILES: CCCN(CCC)CCOc1ccc(C(=O)NC2C/C(=C\c3cc(Cl)cc(Cl)c3)C(=O)/C(=C/c3cc(Cl)cc(Cl)c3)C2)cc1]

### N-(3,5-Bis((E)-3,5-dichlorobenzylidene)-4-oxocyclohexyl)-4-(2-(dipropylamino)ethoxy)benzamide (JCS062)

¹H NMR (500 MHz, CDCl₃) δ 7.68 (s, 2H), 7.64 (d, J=8.8 Hz, 2H), 7.32-7.31 (m, 2H), 7.23-7.22 (m, 4H), 6.87 (d, J=8.8 Hz, 2H), 6.27 (d, J=7.3 Hz, 1H), 4.54-4.47 (m, 1H), 4.02 (t, J=6.3 Hz, 2H), 3.20 (bd, J=15.4 Hz, 2H), 3.07 (dd, J=15.3, 7.4 Hz, 2H), 2.84 (t, J=6.2 Hz, 2H), 2.52-2.40 (m, 4H), 1.54-1.40 (m, 4H), 0.87 (t, J=7.3 Hz, 6H). ¹³C NMR (126 MHz, CDCl₃) δ 187.6, 166.7, 161.9, 137.9 (2C), 137.4 (2C), 135.3, 134.2 (2C), 129.1 (2C), 128.8 (2C), 128.2, 126.0 (2C), 114.47, 67.0, 57.1, 52.8, 44.4, 33.7, 20.5, 11.9. HR-APCI m/z calcd for C₃₅H₃₇C₁₄N₂O₃ [M+H]=673.1552, found 673.1532.

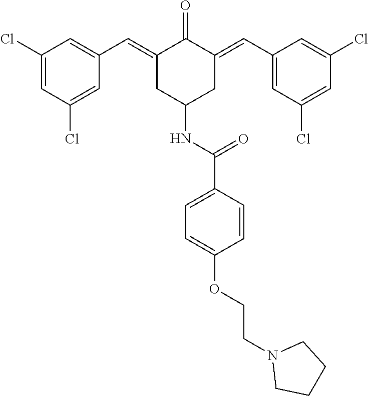 [SMILES: O=C1/C(=C/c2cc(Cl)cc(Cl)c2)CC(NC(=O)c2ccc(OCCN3CCCC3)cc2)C/C1=C\c1cc(Cl)cc(Cl)c1]

### N-(3,5-Bis((E)-3,5-dichlorobenzylidene)-4-oxocyclohexyl)-4-(2-(pyrrolidin-1-yl)ethoxy)benzamide (JCS064)

¹H NMR (500 MHz, CDCl₃) δ 7.73 (s, 2H), 7.63 (d, J=8.3 Hz, 2H), 7.36-7.32 (m, 2H), 7.27-7.24 (m, 4H), 6.90 (d, J=8.6 Hz, 2H), 6.11 (d, J=7.5 Hz, 1H), 4.53-4.48 (m, 1H), 4.11 (t, J=6.0 Hz, 2H), 3.21 (bd, J=15.7 Hz, 2H), 3.06 (dd, J=15.7, 7.9 Hz, 2H), 2.89 (t, J=5.9 Hz, 2H), 2.60 (t, J=5.0 Hz, 4H), 1.80 (t, J=5.0 Hz, 6H). ¹³C NMR (126 MHz, CDCl₃) δ 187.6, 166.7, 161.9, 138.0 (2C), 137.5 (2C), 135.4, 134.2 (2C), 129.1 (2C), 128.8 (2C), 128.3, 126.2 (2C), 114.5, 67.4, 55.0, 54.9, 44.4, 33.8, 23.6. HR-APCI m/z calcd for C₃₃H₃₁C₁₄N₂O₃ [M+H]=643.1083, found 643.1065.

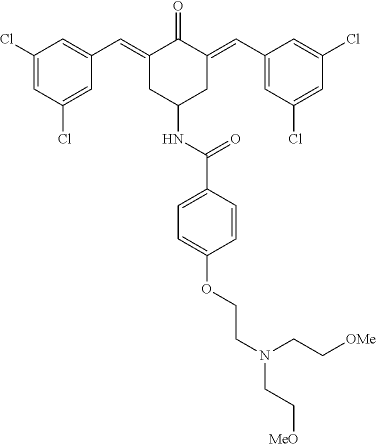 [SMILES: COCCN(CCOC)CCOc1ccc(C(=O)NC2C/C(=C\c3cc(Cl)cc(Cl)c3)C(=O)/C(=C/c3cc(Cl)cc(Cl)c3)C2)cc1]

### N-(3,5-Bis((E)-3,5-dichlorobenzylidene)-4-oxocyclohexyl)-4-(2-(bis(2-methoxyethyl)amino)ethoxy)-benzamide (JCS068)

¹H NMR (500 MHz, CDCl₃) δ 7.75 (s, 2H), 7.62 (d, J=8.3 Hz, 2H), 7.37-7.33 (m, 2H), 7.27-7.26 (m, 4H), 6.89 (d, J=8.6 Hz, 2H), 6.03 (d, J=7.3 Hz, 1H), 4.55-4.48 (m, 1H), 4.07 (t, J=6.1 Hz, 2H), 3.48 (t, J=5.8 Hz, 4H), 3.33 (s, 6H), 3.22 (bd, J=15.9 Hz, 2H), 3.10-3.00 (m, 4H), 2.83 (t, J=5.9 Hz, 4H). ¹³C NMR (126 MHz, CDCl₃) δ 187.5, 166.5, 161.7, 137.8 (2C), 137.4 (2C), 135.2, 134.0 (2C), 129.0 (2C), 128.7 (2C), 128.1, 126.0 (2C), 114.3, 71.2, 66.8, 58.8, 54.7, 53.8, 44.3, 33.7. HR-APCI m/z calcd for C₃₅H₃₇C₁₄N₂O₅ [M+H]=707.1451, found 707.1435.

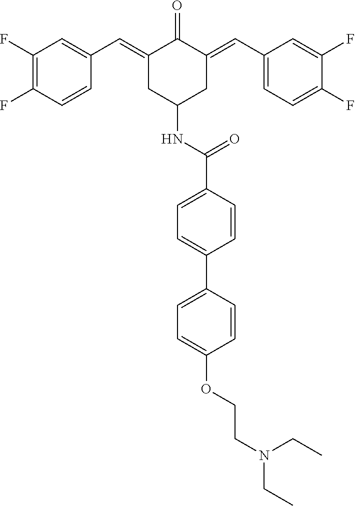 [SMILES: CCN(CC)CCOc1ccc(-c2ccc(C(=O)NC3C/C(=C\c4ccc(F)c(F)c4)C(=O)/C(=C/c4ccc(F)c(F)c4)C3)cc2)cc1]

### N-(3,5-Bis((E)-3,4-difluorobenzylidene)-4-oxocyclohexyl)-4′-(2-(diethylamino)ethoxy)-[1,1′-biphenyl]-4-carboxamide (JCSO95)

¹H NMR (500 MHz, CDCl3) δ 7.81 (s, 2H), 7.73 (d, J=8.3 Hz, 2H), 7.56 (d, J=8.2 Hz, 2H), 7.50 (d, J=8.7 Hz, 2H), 7.28-7.17 (m, 6H), 6.96 (d, J=8.7 Hz, 2H), 6.30 (d, J=7.3 Hz, 1H), 4.56-4.50 (m, 1H), 4.17 (t, J=6.0 Hz, 2H), 3.28 (br d, J=15.2 Hz, 2H), 3.09 (dd, J=15.8, 8.1 Hz, 2H), 3.02 (t, J=5.4 Hz, 2H), 2.78 (q, J=7.0 Hz, 4H), 1.15 (t, J=7.1 Hz, 6H).

¹³C NMR (126 MHz, CDCl₃) δ 187.8, 166.8, 158.8, 150.6 (dd, J_{CF}=253.2 Hz, J_{C-CF}=12.6 Hz, 2C), 150.2 (dd, J_{CF}=249.4 Hz, J_{C-CF}=12.6 Hz, 2C), 144.2, 137.9 (2C), 132.6 (2C), 132.2, 132.1 (dd, J_{C-C-CF}=5.0 Hz, J_{C-C-CF}=3.7 Hz, 2C), 131.8, 128.2 (2C), 127.4 (2C), 127.0 (dd, J_{C-C-CF}=7.5 Hz, J_{C-C-CF}=3.7 Hz, 2C), 126.7 (2C), 118.9 (d, J_{C-CF}=17.6 Hz, 2C), 117.6 (d, J_{C-CF}=17.6 Hz, 2C), 114.9 (2C), 66.2, 51.5, 47.7, 44.6, 33.7, 11.4.

¹⁹F NMR (376 MHz, CDCl₃) δ −135.0 (d, J_{FF}=18.8 Hz, 2F), −136.4 (d, J_{FF}=22.5 Hz, 2F).

HR-APCI m/z calcd for C₃₉H₃₇F₄N₂O₃ [M+H]=657.2695, found 657.2734.

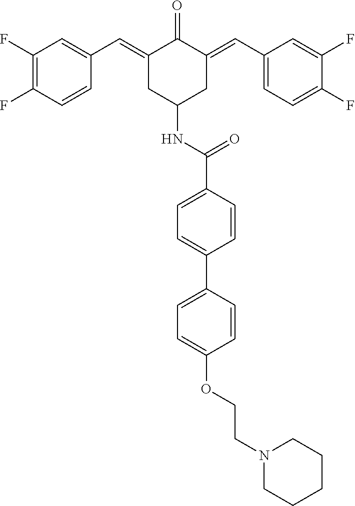 [SMILES: O=C1/C(=C/c2ccc(F)c(F)c2)CC(NC(=O)c2ccc(-c3ccc(OCCN4CCCCC4)cc3)cc2)C/C1=C\c1ccc(F)c(F)c1]

### N-(3,5-Bis((E)-3,4-difluorobenzylidene)-4-oxocyclohexyl)-4′-(2-(piperidin-1-yl)ethoxy)-[1,1′-biphenyl]-4-carboxamide (JCSO96)

¹H NMR (500 MHz, CDCl₃) δ 7.82 (s, 2H), 7.72 (d, J=8.4 Hz, 2H), 7.57 (d, J=8.4 Hz, 2H), 7.51 (d, J=8.7 Hz, 2H), 7.28-7.19 (m, 6H), 6.98 (d, J=8.7 Hz, 2H), 6.21 (d, J=7.3 Hz, 1H), 4.56-4.52 (m, 1H), 4.25 (t, J=6.0 Hz, 2H), 3.28 (br d, J=17.5 Hz, 2H), 3.10 (t, J=5.4 Hz, 2H), 2.74-2.59 (m, 4H), 1.72-1.70 (m, 4H), 1.55-1.46 (m, 2H).

¹³C NMR (126 MHz, CDCl₃) δ 187.8, 166.8, 158.8, 150.6 (dd, J_{CF}=253.2 Hz, J_{C-CF}=12.6 Hz, 2C), 150.2 (dd, J_{CF}=249.4 Hz, J_{C-CF}=12.6 Hz, 2C), 144.1, 137.9 (2C), 132.6 (2C), 132.2, 132.0 (dd, J_{C-C-CF}=5.0 Hz, J_{C-C-CF}=3.7 Hz, 2C), 131.8, 128.2 (2C), 127.4 (2C), 127.0 (dd, J_{C-C-CF}=7.5 Hz, J_{C-C-CF}=3.7 Hz, 2C), 126.7 (2C), 118.9 (d, J_{C-CF}=17.6 Hz, 2C), 117.6 (d, J_{C-CF}=17.6 Hz, 2C), 115.0 (2C), 65.6, 57.6, 54.9, 44.6, 33.7, 25.5, 23.8.

¹⁹F NMR (376 MHz, CDCl₃) δ −135.0 (d, J_{FF}=18.8 Hz, 2F), −136.4 (d, J_{FF}=22.5 Hz, 2F).

HR-APCI m/z calcd for C₄₀H₃₇F₄N₂O₃ [M+H]=669.2670, found 669.2734.

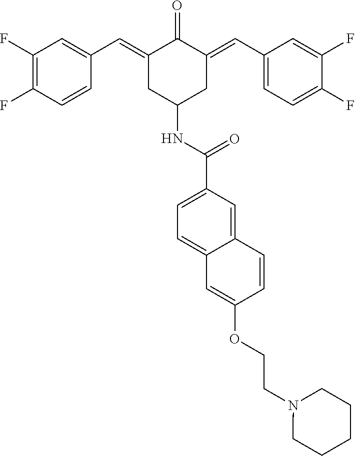 [SMILES: O=C1/C(=C/c2ccc(F)c(F)c2)CC(NC(=O)c2ccc3cc(OCCN4CCCCC4)ccc3c2)C/C1=C\c1ccc(F)c(F)c1]

### N-(3,5-Bis((E)-3,4-difluorobenzylidene)-4-oxocyclohexyl)-6-(2-(piperidin-1-yl)ethoxy)-2-naphth-amide (JCSO97)

¹H NMR (500 MHz, CDCl₃) δ 8.13 (s, 1H), 7.78 (s, 2H), 7.74 (d, J=9.0 Hz, 1H), 7.69 (s, 2H), 7.28-7.13 (m, 7H), 7.10 (d, J=2.5 Hz, 1H), 6.50 (d, J=7.3 Hz, 1H), 4.57-4.49 (m, 1H), 4.26 (t, J=5.8 Hz, 2H), 3.29 (br d, J=15.4 Hz, 2H), 3.09 (dd, J=16.6, 9.3 Hz, 2H), 2.91 (t, J=5.8 Hz, 2H), 2.64 (t, J=5.2 Hz, 4H), 1.70-1.65 (m, 4H), 1.50-1.44 (m, 2H).

¹³C NMR (126 MHz, CDCl₃) δ 187.9, 167.3, 158.2, 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 137.9 (2C), 136.4, 132.9 (2C), 132.2 (dd, J_{C-C-CF}=5.0 Hz, J_{C-C-CF}=3.7 Hz, 2C), 130.5, 129.0, 128.0, 127.5, 127.3, 127.2 (dd, J_{C-C-CF}=7.5 Hz, J_{C-C-CF}=3.7 Hz, 2C), 124.1, 120.1, 119.1 (d, J_{C-CF}=17.6 Hz, 2C), 117.7 (d, J_{C-CF}=17.6 Hz, 2C), 106.6, 77.4, 65.7, 57.7, 55.0, 33.8, 25.5, 23.9.

¹⁹F NMR (376 MHz, CDCl₃) δ −135.0 (d, J_{FF}=18.8 Hz, 2F), −136.5 (d, J_{FF}=22.5 Hz, 2F).

HR-APCI m/z calcd for C₃₈H₃₅F₄N₂O₃ [M+H]=643.2545, found 643.2578.

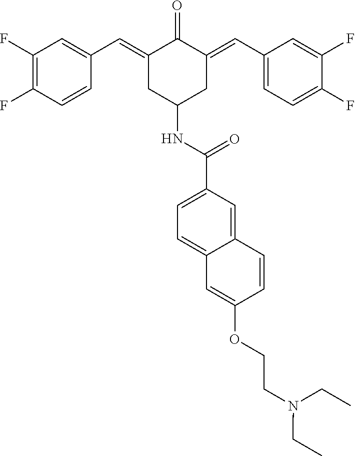 [SMILES: CCN(CC)CCOc1ccc2cc(C(=O)NC3C/C(=C\c4ccc(F)c(F)c4)C(=O)/C(=C/c4ccc(F)c(F)c4)C3)ccc2c1]

### N-(3,5-Bis((E)-3,4-difluorobenzylidene)-4-oxocyclohexyl)-6-(2-(diethylamino)ethoxy)-2-naphtha-mide (JCSO98)

¹H NMR (500 MHz, CDCl₃) δ 8.13 (s, 1H), 7.75 (s, 2H), 7.73-7.64 (m, 3H), 7.27-7.08 (m, 8H), 6.62 (d, J=7.3 Hz, 1H), 4.55-4.48 (m, 1H), 4.18 (t, J=6.0 Hz, 2H), 3.26 (br d, J=15.4 Hz, 2H), 3.07 (dd, J=16.3, 7.8 Hz, 2H), 2.97 (t, J=6.0 Hz, 2H), 2.71 (q, J=7.1 Hz, 4H), 1.11 (t, J=7.2 Hz, 6H).

¹³C NMR (126 MHz, CDCl₃) δ 187.9, 167.3, 158.3, 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 137.8 (2C), 136.3, 132.9 (2C), 132.2 (dd, J_{C-C-CF}=5.0 Hz, J_{C-C-CF}=3.7 Hz, 2C), 130.4, 129.0, 127.9, 127.5, 127.2, 127.1 (dd, J_{C-C-CF}=7.5 Hz, J_{C-C-CF}=3.7 Hz, 2C), 124.1, 120.1, 118.9 (d, J_{C-CF}=17.6 Hz, 2C), 117.7 (d, J_{C-CF}=17.6 Hz, 2C), 106.5, 66.4, 51.5, 47.9, 44.9, 33.8, 11.5.

¹⁹F NMR (376 MHz, CDCl₃) δ −135.0 (d, J_{FF}=18.8 Hz, 2F), −136.5 (d, J_{FF}=22.5 Hz, 2F).

HR-APCI m/z calcd for C₃₇H₃₅F₄N₂O₃ [M+H]=631.2536, found 631.2573.

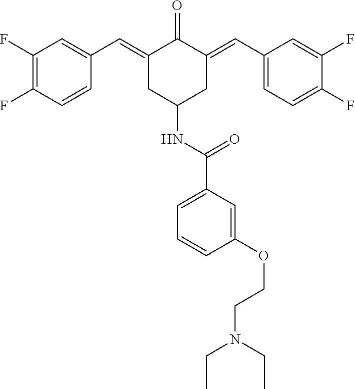 [SMILES: CCN(CC)CCOc1cccc(C(=O)NC2C/C(=C\c3ccc(F)c(F)c3)C(=O)/C(=C/c3ccc(F)c(F)c3)C2)c1]

### N-(3,5-Bis((E)-3,4-difluorobenzylidene)-4-oxocyclohexyl)-3-(2-(diethylamino)ethoxy)benzamide (JCSO99)

¹H NMR (500 MHz, CDCl₃) δ 7.78 (s, 2H), 7.33-7.16 (m, 9H), 7.04-6.97 (m, 1H), 6.41 (d, J=7.4 Hz, 1H), 4.51-4.44 (m, 1H), 4.10 (t, J=6.1 Hz, 2H), 3.26 (br d, J=15.2 Hz, 2H), 3.06 (dd, J=15.8, 8.6 Hz, 2H), 2.89 (t, J=6.1 Hz, 2H), 2.67 (q, J=7.2 Hz, 4H), 1.08 (t, J=7.2 Hz, 7H).

¹³C NMR (126 MHz, CDCl₃) δ 187.7, 166.8, 158.9, 150.6 (dd, J_{CF}=253.2 Hz, J_{C-CF}=12.6 Hz, 2C), 150.2 (dd, J_{CF}=249.4 Hz, J_{C-CF}=12.6 Hz, 2C), 137.7 (2C), 135.3, 132.7 (2C), 132.1 (dd, J_{C-C-CF}=5.0 Hz, J_{C-C-CF}=3.7 Hz, 2C), 129.6, 127.0 (dd, J_{C-C-CF}=7.5 Hz, J_{C-C-CF}=3.7 Hz, 2C), 119.0, 118.9 (d, J_{C-CF}=17.6 Hz, 2C), 118.3, 117.6 (d, J_{C-CF}=17.6 Hz, 2C), 113.0, 66.3, 51.5, 47.7, 44.7, 33.7, 11.4.

¹⁹F NMR (376 MHz, CDCl₃) δ −135.1 (d, J_{FF}=18.8 Hz, 2F), −136.5 (d, J_{FF}=22.5 Hz, 2F).

HR-APCI m/z calcd for C₃₃H₃₃F₄N₂O₃ [M+H]=581.2422, found 581.2421.

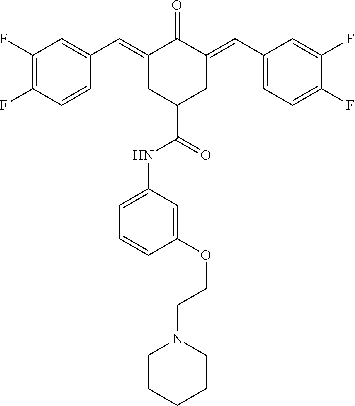 [SMILES: O=C1/C(=C/c2ccc(F)c(F)c2)CC(C(=O)Nc2cccc(OCCN3CCCCC3)c2)C/C1=C\c1ccc(F)c(F)c1]

### N-(3,5-Bis((E)-3,4-difluorobenzylidene)-4-oxocyclohexyl)-3-(2-(piperidin-1-yl)ethoxy)benzamide (JCS100)

¹H NMR (500 MHz, CDCl₃) δ 7.78 (s, 2H), 7.33-7.17 (m, 9H), 7.02-7.00 (m, 1H), 6.40 (d, J=7.4 Hz, 1H), 4.51-4.44 (m, 1H), 4.16 (t, J=6.0 Hz, 2H), 3.26 (br d, J=14.6 Hz, 2H), 3.06 (dd, J=15.8, 8.7 Hz, 2H), 2.81 (t, J=6.1 Hz, 2H), 2.56 (t, J=5.5 Hz, 4H), 1.69-1.61 (m, 4H), 1.49-1.43 (m, 2H).

¹³C NMR (126 MHz, CDCl₃) δ 187.7, 166.7, 158.8, 150.6 (dd, J_{CF}=253.2 Hz, J_{C-CF}=12.6 Hz, 2C), 150.2 (dd, J_{CF}=249.4 Hz, J_{C-CF}=12.6 Hz, 2C), 137.7 (2C), 135.3, 132.7 (2C), 132.1 (dd, J_{C-C-CF}=5.0 Hz, J_{C-C-CF}=3.7 Hz, 2C), 129.6, 127.0 (dd, J_{C-C-CF}=7.5 Hz, J_{C-C-CF}=3.7 Hz, 2C), 119.0, 118.9 (d, J_{C-CF}=17.6 Hz, 2C), 118.3, 117.6 (d, J_{C-CF}=17.6 Hz, 2C), 113.1, 65.7, 57.6, 55.0, 44.8, 33.7, 25.5, 23.9.

¹⁹F NMR (376 MHz, CDCl₃) δ −135.1 (d, J_{FF}=18.8 Hz, 2F), −136.5 (d, J_{FF}=22.5 Hz, 2F).

HR-APCI m/z calcd for C₃₄H₃₃F₄N₂O₃ [M+H]=593.2455, found 593.2421.

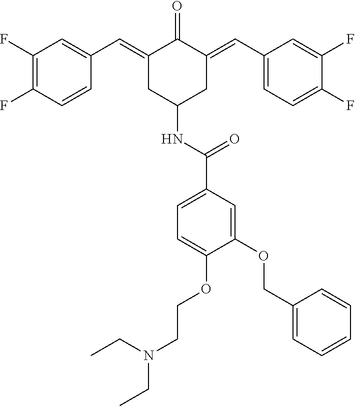 [SMILES: CCN(CC)CCOc1ccc(C(=O)NC2C/C(=C\c3ccc(F)c(F)c3)C(=O)/C(=C/c3ccc(F)c(F)c3)C2)cc1OCc1ccccc1]

### 3-(Benzyloxy)-N-(3,5-bis((E)-3,4-difluorobenzylidene)-4-oxocyclohexyl)-4-(2-(diethylamino)ethoxy)-benzamide (JCS101)

¹H NMR (500 MHz, CDCl₃) δ 7.78 (s, 2H), 7.41-7.14 (m, 13H), 6.84 (d, J=8.4 Hz, 1H), 6.19 (d, J=7.3 Hz, 1H), 5.13 (s, 2H), 4.51-4.44 (m, 1H), 4.11 (t, J=6.3 Hz, 2H), 3.25 (br d, J=14.1 Hz, 2H), 3.04 (dd, J=15.8, 8.3 Hz, 2H), 2.91 (t, J=6.2 Hz, 2H), 2.62 (q, J=7.2 Hz, 4H), 1.03 (t, J=7.1 Hz, 6H).

¹³C NMR (126 MHz, CDCl₃) δ 187.7, 166.6, 151.6, 150.7 (dd, J_{CF}=211.6 Hz, J_{C-CF}=11.3 Hz, 2C), 150.2 (dd, J_{CF}=219.2 Hz, J_{C-CF}=10.0 Hz, 2C), 137.9 (2C), 136.5, 132.8 (2C), 132.2 (dd, J_{C-C-CF}=5.0 Hz, J_{C-C-CF}=3.7 Hz, 2C), 128.6 (2C), 128.1, 127.3 (2C), 127.1 (dd, J_{C-C-CF}=7.5 Hz, J_{C-C-CF}=3.7 Hz, 2C), 119.7, 119.0 (d, J_{C-CF}=17.6 Hz, 2C), 117.7 (d, J_{C-CF}=17.6 Hz, 2C), 113.2, 112.7, 70.9, 66.3, 51.5, 47.7, 44.7, 33.7, 11.4.

¹⁹F NMR (376 MHz, CDCl₃) δ −135.0 (d, J_{FF}=18.8 Hz, 2F), −136.5 (d, J_{FF}=22.5 Hz, 2F).

HR-APCI m/z calcd for C₄₀H₃₉F₄N₂O₄ [M+H]=687.2863, found 687.2840.

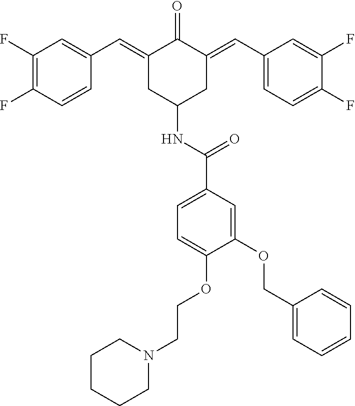 [SMILES: O=C1/C(=C/c2ccc(F)c(F)c2)CC(NC(=O)c2ccc(OCCN3CCCCC3)c(OCc3ccccc3)c2)C/C1=C\c1ccc(F)c(F)c1]

### 3-(Benzyloxy)-N-(3,5-bis((E)-3,4-difluorobenzylidene)-4-oxocyclohexyl)-4-(2-(piperidin-1-yl)eth-oxy)benzamide (JCS102)

¹H NMR (500 MHz, CDCl₃) δ 7.78 (s, 2H), 7.42-7.15 (m, 13H), 6.84 (d, J=8.4 Hz, 1H), 6.20 (d, J=7.3 Hz, 1H), 5.14 (s, 2H), 4.51-4.44 (m, 1H), 4.18 (t, J=6.1 Hz, 2H), 3.25 (br d, J=15.3 Hz, 2H), 3.04 (dd, J=15.5, 8.4 Hz, 2H), 2.11 (t, J=6.2 Hz, 2H), 2.59-2.47 (m, 4H), 1.60-1.56 (m, 4H), 1.45-1.40 (m, 2H).

¹³C NMR (126 MHz, CDCl₃) δ 187.9, 166.6, 151.7, 150.7 (dd, J_{CF}=211.6 Hz, J_{C-CF}=11.3 Hz, 2C), 150.2 (dd, J_{CF}=219.2 Hz, J_{C-CF}=10.0 Hz, 2C), 149.0, 137.8 (2C), 136.6, 132.8 (2C), 132.2 (dd, J_{C-C-CF}=5.0 Hz, J_{C-C-CF}=3.7 Hz, 2C), 128.6 (2C), 128.1, 127.3 (2C), 127.2 (dd, J_{C-C-CF}=7.5 Hz, J_{C-C-CF}=3.7 Hz, 2C), 127.1, 119.9, 119.0 (d, J_{C-CF}=17.6 Hz, 2C), 117.7 (d, J_{C-CF}=17.6 Hz, 2C), 113.4, 113.1, 70.9, 67.5, 57.8, 55.1, 44.8, 33.8, 26.0, 24.2.

¹⁹F NMR (376 MHz, CDCl₃) δ −135.0 (d, J_{FF}=18.8 Hz, 2F), −136.5 (d, J_{FF}=22.5 Hz, 2F).

HR-APCI m/z calcd for C₄₁H₃₉F₄N₂O₄ [M+H]=699.2863, found 699.2840.

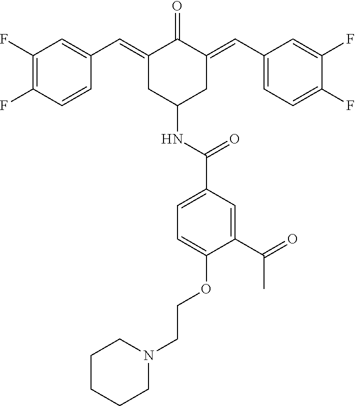 [SMILES: CC(=O)c1cc(C(=O)NC2C/C(=C\c3ccc(F)c(F)c3)C(=O)/C(=C/c3ccc(F)c(F)c3)C2)ccc1OCCN1CCCCC1]

### 3-Acetyl-N-(3,5-bis((E)-3,4-difluorobenzylidene)-4-oxocyclohexyl)-4-(2-(piperidin-1-yl)ethoxy)-benzamide (JCS103)

¹H NMR (500 MHz, CDCl₃) δ 8.16-8.04 (m, 2H), 7.85 (s, 2H), 7.37-7.20 (m, 6H), 7.08 (d, J=8.7 Hz, 1H), 6.67 (d, J=7.3 Hz, 1H), 4.51-4.44 (m, 1H), 4.30 (t, J=6.0 Hz, 2H), 3.38 (br d, J=15.7 Hz, 2H), 3.08 (dd, J=16.4, 9.4 Hz, 2H), 2.89 (t, J=6.0 Hz, 2H), 2.72 (s, 3H), 2.62-2.48 (m, 4H), 1.69-1.64 (m, 4H), 1.55-1.50 (m, 2H).

¹³C NMR (126 MHz, CDCl₃) δ 199.2, 187.8, 165.7, 160.9, 150.7 (dd, J_{CF}=211.6 Hz, J_{C-CF}=11.3 Hz, 2C), 150.2 (dd, J_{CF}=219.2 Hz, J_{C-CF}=10.0 Hz, 2C), 137.6 (2C), 134.0, 133.0 (2C), 132.2 (dd, J_{C-C-CF}=5.0 Hz, J_{C-C-CF}=3.7 Hz, 2C), 128.3, 127.5, 127.1 (dd, J_{C-C-CF}=7.5 Hz, J_{C-C-CF}=3.7 Hz, 2C), 126.1, 119.0 (d, J_{C-CF}=17.6 Hz, 2C), 117.7 (d, J_{C-CF}=17.6 Hz, 2C), 112.9, 67.1, 57.6, 55.1, 45.1, 34.0, 32.1, 26.0, 24.2.

¹⁹F NMR (376 MHz, CDCl₃) δ −135.1 (d, J_{FF}=18.8 Hz, 2F), −136.4 (d, J_{FF}=22.5 Hz, 2F).

HR-APCI m/z calcd for C₃₆H₃₅F₄N₂O₄ [M+H]=635.2540, found 635.2527.

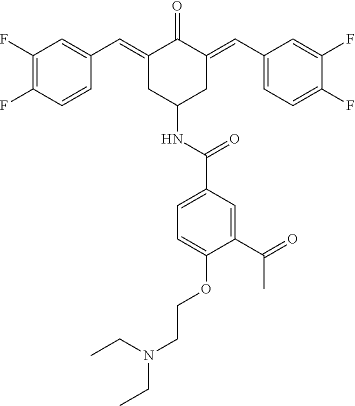 [SMILES: CCN(CC)CCOc1ccc(C(=O)NC2C/C(=C\c3ccc(F)c(F)c3)C(=O)/C(=C/c3ccc(F)c(F)c3)C2)cc1C(C)=O]

### 3-Acetyl-N-(3,5-bis((E)-3,4-difluorobenzylidene)-4-oxocyclohexyl)-4-(2-(diethylamino)ethoxy)-benzamide (JCS104)

¹H NMR (500 MHz, CDCl₃) δ 8.03-7.93 (m, 2H), 7.73 (s, 2H), 7.26-7.13 (m, 6H), 6.96 (d, J=8.8 Hz, 1H), 6.71 (d, J=7.3 Hz, 1H), 4.51-4.39 (m, 1H), 4.17 (t, J=6.3 Hz, 2H), 3.27 (br d, J=15.5 Hz, 2H), 3.03-2.96 (m, 2H), 2.93 (t, J=6.2 Hz, 2H), 2.64 (q, J=7.2 Hz, 4H), 2.59 (s, 3H), 1.04 (t, J=7.1 Hz, 6H).

¹³C NMR (126 MHz, CDCl₃) δ 199.0, 187.7, 165.6, 160.6, 150.7 (dd, J_{CF}=211.6 Hz, J_{C-CF}=11.3 Hz, 2C), 150.2 (dd, J_{CF}=219.2 Hz, J_{C-CF}=10.0 Hz, 2C), 137.4 (2C), 133.8, 133.0 (2C), 132.1 (dd, J_{C-C-CF}=5.0 Hz, J_{C-C-CF}=3.7 Hz, 2C), 128.4, 127.4, 127.0 (dd, J_{C-C-CF}=7.5 Hz, J_{C-C-CF}=3.7 Hz, 2C), 126.1, 118.9 (d, J_{C-CF}=17.6 Hz, 2C), 117.6 (d, J_{C-CF}=17.6 Hz, 2C), 112.8, 67.4, 51.7, 47.6, 45.0, 33.8, 31.9, 11.6.

¹⁹F NMR (376 MHz, CDCl₃) δ −135.2 (d, J_{FF}=18.8 Hz, 2F), −136.5 (d, J_{FF}=22.5 Hz, 2F).

HR-APCI m/z calcd for C₃₅H₃₅F₄N₂O₄ [M+H]=623.2545, found 623.2527.

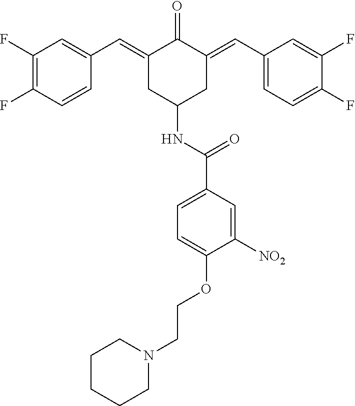 [SMILES: O=C1/C(=C/c2ccc(F)c(F)c2)CC(NC(=O)c2ccc(OCCN3CCCCC3)c([N+](=O)[O-])c2)C/C1=C\c1ccc(F)c(F)c1]

### N-(3,5-Bis((E)-3,4-difluorobenzylidene)-4-oxocyclohexyl)-3-nitro-4-(2-(piperidin-1-yl)ethoxy)benzamide (JCS105)

¹H NMR (500 MHz, CDCl₃) δ 8.19 (s, 1H), 7.96 (d, J=8.8 Hz, 1H), 7.75 (s, 2H), 7.29-7.15 (m, 6H), 7.08 (d, J=8.9 Hz, 1H), 6.72 (d, J=7.3 Hz, 1H), 4.51-4.44 (m, 1H), 4.27 (t, J=5.8 Hz, 2H), 3.29 (br d, J=15.1 Hz, 2H), 3.05 (dd, J=16.3, 8.4 Hz, 2H), 2.85 (t, J=5.8 Hz, 2H), 2.55 (t, J=5.4 Hz, 4H), 1.61-1.56 (m, 4H), 1.55-1.50 (m, 2H).

¹³C NMR (126 MHz, CDCl₃) δ 187.8, 164.5, 154.7, 150.7 (dd, J_{CF}=254.5 Hz, J_{C-CF}=12.6 Hz, 2C), 150.2 (dd, J_{CF}=249.4 Hz, J_{C-CF}=12.6 Hz, 2C), 139.0, 137.9 (2C), 133.6, 132.7 (2C), 132.1 (dd, J_{C-C-CF}=5.0 Hz, J_{C-C-CF}=3.7 Hz, 2C), 127.2 (dd, J_{C-C-CF}=7.5 Hz, J_{C-C-CF}=3.7 Hz, 2C), 126.1, 124.4, 118.9 (d, J_{C-CF}=17.6 Hz, 2C), 117.7 (d, J_{C-CF}=17.6 Hz, 2C), 114.6, 68.5, 57.2, 55.2, 45.2, 33.7, 25.8, 23.9.

¹⁹F NMR (376 MHz, CDCl₃) δ −134.9 (d, J_{FF}=18.8 Hz, 2F), −136.4 (d, J_{FF}=22.5 Hz, 2F).

HR-APCI m/z calcd for C₃₄H₃₂F₄N₃O₅ [M+H]=638.2268, found 638.2272.

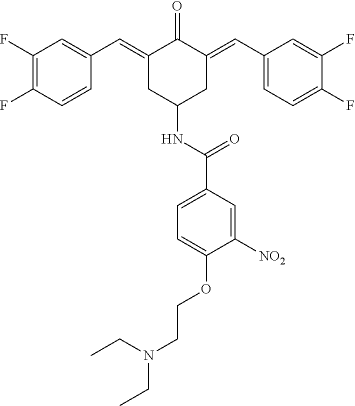 [SMILES: CCN(CC)CCOc1ccc(C(=O)NC2C/C(=C\c3ccc(F)c(F)c3)C(=O)/C(=C/c3ccc(F)c(F)c3)C2)cc1[N+](=O)[O-]]

### N-(3,5-Bis((E)-3,4-difluorobenzylidene)-4-oxocyclohexyl)-4-(2-(diethylamino)ethoxy)-3-nitrobenzamide (JCS106)

¹H NMR (500 MHz, CDCl₃) δ 8.18 (s, 1H), 7.95 (d, J=8.0 Hz, 1H), 7.76 (s, 2H), 7.29-7.15 (m, 6H), 7.08 (d, J=8.7 Hz, 1H), 6.62 (d, J=7.1 Hz, 1H), 4.49-4.43 (m, 1H), 4.21 (t, J=5.8 Hz, 2H), 3.29 (br d, J=16.0 Hz, 2H), 3.05 (dd, J=15.5, 7.7 Hz, 2H), 2.95 (t, J=5.8 Hz, 2H), 2.66 (q, J=7.2 Hz, 4H), 1.04 (t, J=7.1 Hz, 6H).

¹³C NMR (126 MHz, CDCl₃) δ 187.8, 164.5, 154.8, 150.7 (dd, J_{CF}=254.5 Hz, J_{C-CF}=12.6 Hz, 2C), 150.2 (dd, J_{CF}=249.4 Hz, J_{C-CF}=12.6 Hz, 2C), 139.0, 137.9 (2C), 133.6, 132.7 (2C), 132.2 (dd, J_{C-C-CF}=5.0 Hz, J_{C-C-CF}=3.7 Hz, 2C), 127.2 (dd, J_{C-C-CF}=7.5 Hz, J_{C-C-CF}=3.7 Hz, 2C), 126.1, 124.4, 119.0 (d, J_{C-CF}=17.6 Hz, 2C), 117.8 (d, J_{C-CF}=17.6 Hz, 2C), 114.5, 69.1, 51.3, 48.1, 45.3, 33.8, 11.7.

¹⁹F NMR (376 MHz, CDCl₃) δ −134.9 (d, J_{FF}=18.8 Hz, 2F), −136.4 (d, J_{FF}=22.5 Hz, 2F).

HR-APCI m/z calcd for C₃₃H₃₂F₄N₃O₅ [M+H]=626.2270, found 626.2272.

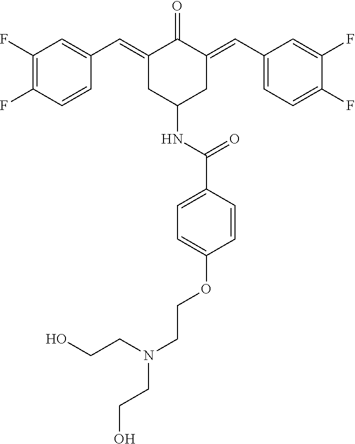 [SMILES: O=C1/C(=C/c2ccc(F)c(F)c2)CC(NC(=O)c2ccc(OCCN(CCO)CCO)cc2)C/C1=C\c1ccc(F)c(F)c1]

### N-(3,5-Bis((E)-3,4-difluorobenzylidene)-4-oxocyclohexyl)-4-(2-(bis(2-hydroxyethyl)amino)ethoxy)-benzamide (JCS107)

¹H NMR (500 MHz, CDCl₃) δ 7.74 (s, 2H), 7.61 (d, J=8.9 Hz, 2H), 7.23-7.11 (m, 6H), 6.82 (d, J=8.8 Hz, 2H), 6.51 (d, J=7.3 Hz, 1H), 4.46-4.39 (m, 1H), 4.02 (t, J=5.4 Hz, 2H), 3.59 (t, J=5.2 Hz, 4H), 3.23 (br d, J=15.2 Hz, 2H), 3.01 (dd, J=16.5, 8.0 Hz, 2H), 2.96 (t, J=5.4 Hz, 2H), 2.75 (t, J=5.2 Hz, 4H).

¹³C NMR (126 MHz, CDCl₃) δ 188.0, 166.7, 161.3, 150.6 (dd, J_{CF}=240.6 Hz, J_{C-CF}=12.6 Hz, 2C), 150.2 (dd, J_{CF}=236.8 Hz, J_{C-CF}=12.6 Hz, 2C), 137.7 (2C), 132.9 (2C), 132.2 (dd, J_{C-C-CF}=5.0 Hz, J_{C-C-CF}=3.7 Hz, 2C), 128.9 (2C), 127.2 (dd, J_{C-C-CF}=7.5 Hz, J_{C-C-CF}=3.7 Hz, 2C), 126.6, 118.9 (d, J_{C-CF}=17.6 Hz, 2C), 117.7 (d, J_{C-CF}=17.6 Hz, 2C), 114.3 (2C), 66.6, 59.7, 57.0, 53.5, 44.8, 33.8.

¹⁹F NMR (376 MHz, CDCl₃) δ −135.0 (d, J_{FF}=18.8 Hz, 2F), −136.5 (d, J_{FF}=22.5 Hz, 2F).

HR-APCI m/z calcd for C₃₃H₃₃F₄N₂O₅ [M+H]=613.2318, found 613.2320.

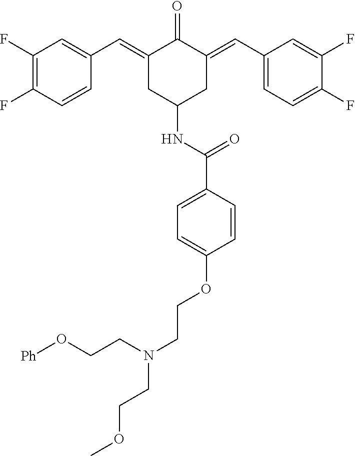 [SMILES: COCCN(CCOc1ccccc1)CCOc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-(2-((2-methoxyethyl)(2-phenoxyethyl)amino)ethoxy)benzamide (JCS108)

¹H NMR (500 MHz, CDCl₃) δ 7.82 (s, 2H), 7.64 (d, J=8.7 Hz, 2H), 7.30-7.19 (m, 8H), 6.97-6.88 (m, 5H), 6.16 (d, J=7.3 Hz, 1H), 4.54-4.49 (m, 1H), 4.10-4.08 (m, 4H), 3.53 (t, J=5.7 Hz, 2H), 3.36 (s, 3H), 3.28 (br d, J=14.0 Hz, 2H), 3.10-3.06 (m, 6H), 2.93 (t, J=5.7 Hz, 2H).

¹³C NMR (126 MHz, CDCl₃) δ 187.9, 166.6, 161.8, 158.8, 150.7 (dd, J_{CF}=211.6 Hz, J_{C-CF}=12.6 Hz, 2C), 150.2 (dd, J_{CF}=219.8 Hz, J_{C-CF}=12.6 Hz, 2C), 137.9 (2C), 132.8 (2C), 132.2 (dd, J_{C-C-CF}=5.0 Hz, J_{C-C-CF}=3.7 Hz, 2C), 129.5 (2C), 128.8 (2C), 127.1 (dd, J_{C-C-CF}=7.5 Hz, J_{C-C-CF}=3.7 Hz, 2C), 126.2, 120.8, 119.0 (d, J_{C-CF}=17.6 Hz, 2C), 117.7 (d, J_{C-CF}=17.6 Hz, 2C), 114.6 (2C), 114.4 (2C), 71.4, 67.1, 66.6, 59.0, 55.0, 54.3, 54.1, 44.6, 33.8.

¹⁹F NMR (376 MHz, CDCl₃) δ −131.2 (d, J_{FF}=18.8 Hz, 2F), −132.6 (d, J_{FF}=22.5 Hz, 2F).

HR-APCI m/z calcd for C₄₀H₃₉F₄N₂O₅ [M+H]=703.2819, found 703.2789.

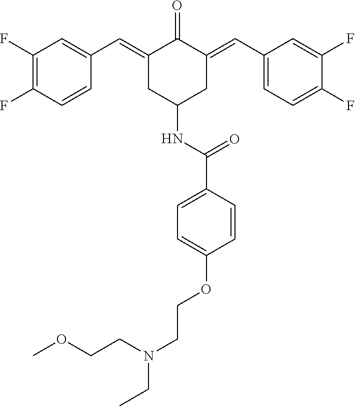 [SMILES: CCN(CCOC)CCOc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-(2-(ethyl(2-methoxyethyl)amino)eth-oxy)benzamide (JCS109)

¹H NMR (500 MHz, CDCl₃) δ 7.79 (s, 2H), 7.65 (d, J=8.5 Hz, 2H), 7.32-7.14 (m, 6H), 6.89 (d, J=8.5 Hz, 2H), 6.23 (d, J=7.3 Hz, 1H), 4.54-4.49 (m, 1H), 4.07 (t, J=6.1 Hz, 2H), 3.49 (t, J=5.8 Hz, 2H), 3.35 (s, 3H), 3.26 (d, J=14.5 Hz, 2H), 3.07 (dd, J=15.5, 8.1 Hz, 2H), 2.94 (t, J=6.1 Hz, 2H), 2.77 (t, J=5.8 Hz, 2H), 2.69 (q, J=7.1 Hz, 2H), 1.07 (t, J=7.1 Hz, 3H).

¹³C NMR (126 MHz, CDCl₃) δ 187.9, 166.7, 161.8, 150.7 (dd, J_{CF}=211.6 Hz, J_{C-CF}=12.6 Hz, 2C), 150.2 (dd, J_{CF}=219.8 Hz, J_{C-CF}=12.6 Hz, 2C), 137.8 (2C), 132.8 (2C), 132.2 (dd, J_{C-C-CF}=5.0 Hz, J_{C-C-CF}=3.7 Hz, 2C), 128.8 (2C), 127.1 (dd, J_{C-C-CF}=7.5 Hz, J_{C-C-CF}=3.7 Hz, 2C), 126.1, 119.0 (d, J_{C-CF}=17.6 Hz, 2C), 117.7 (d, J_{C-CF}=17.6 Hz, 2C), 114.4 (2C), 71.2, 66.9, 58.9, 53.7, 52.7, 49.2, 44.6, 33.8, 11.8.

¹⁹F NMR (376 MHz, CDCl₃) δ −135.0 (d, J_{FF}=18.8 Hz, 2F), −136.5 (d, J_{FF}=22.5 Hz, 2F).

HR-APCI m/z calcd for C₃₄H₃₅F₄N₂O₄ [M+H]=611.2557, found 611.2527.

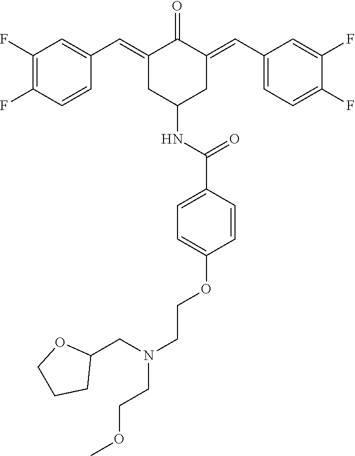 [SMILES: COCCN(CCOc1ccc(C(=O)NC2C/C(=C\c3ccc(F)c(F)c3)C(=O)/C(=C/c3ccc(F)c(F)c3)C2)cc1)CC1CCCO1]

### N-(3,5-Bis((E)-3,4-difluorobenzylidene)-4-oxocyclohexyl)-4-(2-((2-methoxyethyl)((tetrahydrofuran-2-yl)methyl)amino)ethoxy)benzamide (JCS110)

¹H NMR (500 MHz, CDCl₃) δ 7.69 (s, 2H), 7.62 (d, J=8.8 Hz, 2H), 7.23-7.03 (m, 6H), 6.82 (d, J=8.7 Hz, 2H), 6.48 (d, J=7.3 Hz, 1H), 4.46-4.38 (m, 1H), 4.03 (t, J=5.4 Hz, 2H), 3.98-3.92 (m, 1H), 3.80 (td, J=7.0, 7.0 Hz, 1H), 3.68 (td, J=7.5, 7.5 Hz, 1H), 3.45 (t, J=5.5 Hz, 2H), 3.29 (s, 3H), 3.20 (br d, J=13.8 Hz, 2H), 3.03-2.97 (m, 4H), 2.84-2.79 (m, 2H), 2.70-2.64 (m, 2H), 1.95-1.88 (m, 1H), 1.87-1.74 (m, 2H), 1.49-1.42 (m, 1H).

¹³C NMR (126 MHz, CDCl₃) δ 187.8, 166.6, 161.7, 150.5 (dd, J_{CF}=253.3 Hz, J_{C-CF}=12.6 Hz, 2C), 150.1 (dd, J_{CF}=249.4 Hz, J_{C-CF}=12.6 Hz, 2C), 137.5 (2C), 132.8 (2C), 132.1 (dd, J_{C-C-CF}=5.0 Hz, J_{C-C-CF}=3.7 Hz, 2C), 128.7 (2C), 127.1 (dd, J_{C-C-CF}=7.5 Hz, J_{C-C-CF}=3.7 Hz, 2C), 126.0, 118.9 (d, J_{C-CF}=17.6 Hz, 2C), 117.6 (d, J_{C-CF}=17.6 Hz, 2C), 114.3 (2C), 77.9, 71.3, 67.9, 66.9, 59.9, 58.8, 54.9, 53.9, 44.6, 33.7, 30.0, 25.4.

¹⁹F NMR (376 MHz, CDCl₃) δ −135.1 (d, J_{FF}=18.8 Hz, 2F), −136.5 (d, J_{FF}=22.5 Hz, 2F).

HR-APCI m/z calcd for C₃₇H₃₉F₄N₂O₅ [M+H]=667.2817, found 667.2789.

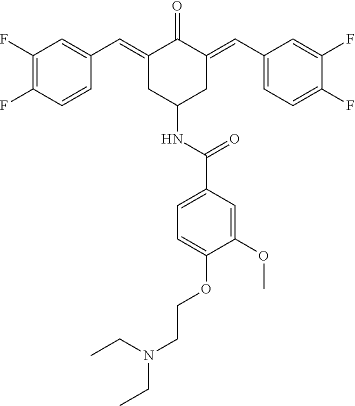 [SMILES: CCN(CC)CCOc1ccc(C(=O)NC2C/C(=C\c3ccc(F)c(F)c3)C(=O)/C(=C/c3ccc(F)c(F)c3)C2)cc1OC]

### N-(3,5-Bis((E)-3,4-difluorobenzylidene)-4-oxocyclohexyl)-4-(2-(diethylamino)ethoxy)-3-methoxy-benzamide (JCS111)

¹H NMR (500 MHz, CDCl₃) δ 7.78 (s, 2H), 7.33 (d, J=2.1 Hz, 1H), 7.27-7.12 (m, 7H), 6.82 (d, J=8.3 Hz, 1H), 6.20 (d, J=7.3 Hz, 1H), 4.49-4.43 (m, 1H), 4.10 (t, J=6.7 Hz, 2H), 3.85 (s, 3H), 3.25 (br d, J=14.2 Hz, 2H), 3.06 (dd, J=15.6, 8.0 Hz, 2H), 2.92 (t, J=6.7 Hz, 2H), 2.64 (q, J=7.1 Hz, 4H), 1.05 (t, J=7.1 Hz, 6H).

¹³C NMR (126 MHz, CDCl₃) δ 187.9, 166.7, 150.7 (dd, J_{CF}=211.6 Hz, J_{C-CF}=12.6 Hz, 2C), 151.5, 150.2 (dd, J_{CF}=207.9 Hz, J_{C-CF}=12.6 Hz, 2C), 149.4, 137.9 (2C), 132.8 (2C), 132.2 (dd, J_{C-C-CF}=5.0 Hz, J_{C-C-CF}=3.7 Hz, 2C), 127.2 (dd, J_{C-C-CF}=7.5 Hz, J_{C-C-CF}=3.7 Hz, 2C), 126.7, 119.2, 119.0 (d, J_{C-CF}=17.6 Hz, 2C), 117.7 (d, J_{C-CF}=17.6 Hz, 2C), 111.7, 111.1, 67.5, 56.1, 51.5, 48.0, 44.7, 33.8, 11.8.

¹⁹F NMR (376 MHz, CDCl₃) δ −135.0 (d, J_{FF}=18.8 Hz, 2F), −136.5 (d, J_{FF}=22.5 Hz, 2F).

HR-APCI m/z calcd for C₃₄H₃₅F₄N₂O₄ [M+H]=611.2557, found 611.2527.

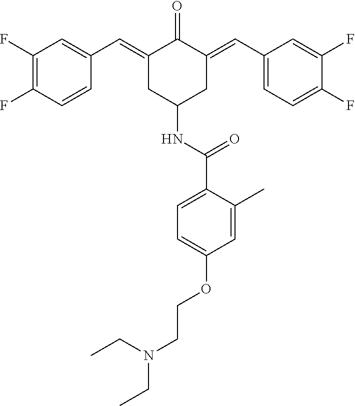 [SMILES: CCN(CC)CCOc1ccc(C(=O)NC2C/C(=C\c3ccc(F)c(F)c3)C(=O)/C(=C/c3ccc(F)c(F)c3)C2)c(C)c1]

### N-(3,5-Bis((E)-3,4-difluorobenzylidene)-4-oxocyclohexyl)-4-(2-(diethylamino)ethoxy)-2-methyl-benzamide (JCS112)

¹H NMR (500 MHz, CDCl₃) δ 7.81 (s, 2H), 7.28-7.20 (m, 7H), 6.69 (s, 1H), 6.67 (d, J=8.5 Hz, 1H), 5.86 (d, J=7.2 Hz, 1H), 4.53-4.49 (m, 1H), 4.04 (t, J=6.2 Hz, 2H), 3.22 (br d, J=15.0 Hz, 2H), 3.15 (dd, J=15.9, 6.9 Hz, 2H), 2.88 (t, J=6.2 Hz, 2H), 2.66 (q, J=7.2 Hz, 4H), 2.30 (s, 3H), 1.08 (t, J=7.2 Hz, 6H).

¹³C NMR (126 MHz, CDCl₃) δ 187.9, 169.4, 160.2, 150.7 (dd, J_{CF}=211.6 Hz, J_{C-CF}=12.6 Hz, 2C), 150.2 (dd, J_{CF}=207.9 Hz, J_{C-CF}=12.6 Hz, 2C), 138.8, 138.1 (2C), 132.7 (2C), 132.2 (dd, J_{C-C-CF}=5.0 Hz, J_{C-C-CF}=3.7 Hz, 2C), 128.6, 128.0, 127.1 (dd, J_{C-C-CF}=7.5 Hz, J_{C-C-CF}=3.7 Hz, 2C), 119.0 (d, J_{C-CF}=17.6 Hz, 2C), 117.7 (d, J_{C-CF}=17.6 Hz, 2C), 117.3, 111.5, 66.6, 51.6, 47.9, 44.4, 33.4, 20.3, 11.8.

¹⁹F NMR (376 MHz, CDCl₃) δ −135.0 (d, J_{FF}=18.8 Hz, 2F), −136.4 (d, J_{FF}=22.5 Hz, 2F).

HR-APCI m/z calcd for C₃₄H₃₅F₄N₂O₃ [M+H]=595.2602, found 595.2578.

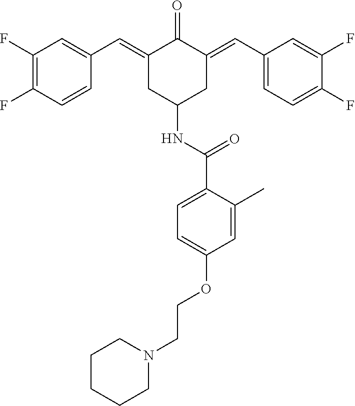 [SMILES: Cc1cc(OCCN2CCCCC2)ccc1C(=O)NC1C/C(=C\c2ccc(F)c(F)c2)C(=O)/C(=C/c2ccc(F)c(F)c2)C1]

### N-(3,5-Bis((E)-3,4-difluorobenzylidene)-4-oxocyclohexyl)-2-methyl-4-(2-(piperidin-1-yl)ethoxy)-benzamide (JCS113)

¹H NMR (500 MHz, CDCl₃) δ 7.83 (s, 2H), 7.31-7.20 (m, 7H), 6.71 (s, 1H), 6.68 (d, J=8.3 Hz, 1H), 5.77 (d, J=7.2 Hz, 1H), 4.55-4.51 (m, 1H), 4.10 (t, J=6.0 Hz, 2H), 3.23 (br d, J=14.1 Hz, 2H), 3.16 (dd, J=16.0, 6.9 Hz, 2H), 2.78 (t, J=6.0 Hz, 2H), 2.58-2.49 (m, 4H), 2.31 (s, 3H), 1.66-1.59 (m, 4H), 1.50-1.44 (m, 2H).

¹³C NMR (126 MHz, CDCl₃) δ 187.9, 169.4, 160.2, 150.8 (dd, J_{CF}=211.6 Hz, J_{C-CF}=12.6 Hz, 2C), 150.3 (dd, J_{CF}=207.9 Hz, J_{C-CF}=12.6 Hz, 2C), 138.7, 138.2 (2C), 132.6 (2C), 132.2 (dd, J_{C-C-CF}=5.0 Hz, J_{C-C-CF}=3.7 Hz, 2C), 128.6, 128.0, 127.1 (dd, J_{C-C-CF}=7.5 Hz, J_{C-C-CF}=3.7 Hz, 2C), 119.0 (d, J_{C-CF}=17.6 Hz, 2C), 117.7 (d, J_{C-CF}=17.6 Hz, 2C), 117.4, 111.6, 66.0, 57.8, 55.1, 44.4, 33.4, 25.9, 24.2, 20.3.

¹⁹F NMR (376 MHz, CDCl₃) δ −131.3 (d, J_{FF}=18.8 Hz, 2F), −132.7 (d, J_{FF}=22.5 Hz, 2F).

HR-APCI m/z calcd for C₃₅H₃₅F₄N₂O₃ [M+H]=607.2607, found 607.2578.

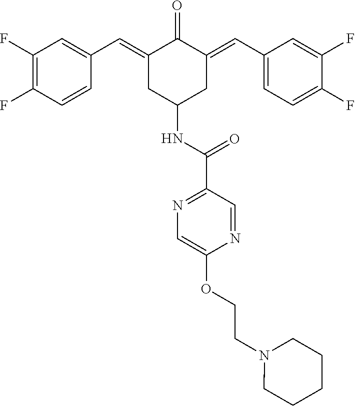 [SMILES: O=C1/C(=C/c2ccc(F)c(F)c2)CC(NC(=O)c2cnc(OCCN3CCCCC3)cn2)C/C1=C\c1ccc(F)c(F)c1]

### N-(3,5-Bis((E)-3,4-difluorobenzylidene)-4-oxocyclohexyl)-5-(2-(piperidin-1-yl)ethoxy)pyrazine-2-carboxamide (JCS114)

¹H NMR (500 MHz, CDCl₃) δ 8.12 (s, 1H), 7.97 (s, 1H), 7.83 (s, 2H), 7.53 (d, J=7.9 Hz, 1H), 7.31-7.18 (m, 6H), 4.51-4.40 (m, 1H), 4.00 (t, J=6.0 Hz, 2H), 3.29 (br d, J=16.0 Hz, 2H), 3.04 (dd, J=16.8, 10.0 Hz, 2H), 2.61 (t, J=6.0 Hz, 2H), 2.42-2.38 (m, 4H), 1.53-1.50 (m, 4H), 1.42-1.38 (m, 2H).

¹³C NMR (126 MHz, CDCl₃) δ 187.7, 162.5, 156.2, 150.7 (dd, J_{CF}=211.6 Hz, J_{C-CF}=12.6 Hz, 2C), 150.2 (dd, J_{CF}=207.9 Hz, J_{C-CF}=12.6 Hz, 2C), 145.9, 137.8 (2C), 133.1, 132.8 (2C), 132.3 (dd, J_{C-C-CF}=5.0 Hz, J_{C-C-CF}=3.7 Hz, 2C), 127.2 (dd, J_{C-C-CF}=7.5 Hz, J_{C-C-CF}=3.7 Hz, 2C), 125.6, 119.0 (d, J_{C-CF}=17.6 Hz, 2C), 117.7 (d, J_{C-CF}=17.6 Hz, 2C), 56.4, 54.6, 46.7, 44.3, 34.0, 26.0, 24.2.

¹⁹F NMR (376 MHz, CDCl₃) δ −131.1 (d, J_{FF}=18.8 Hz, 2F), −132.5 (d, J_{FF}=22.5 Hz, 2F).

HR-APCI m/z calcd for C₃₂H₃₁F₄N₄O₃ [M+H]=595.2353, found 595.2353.

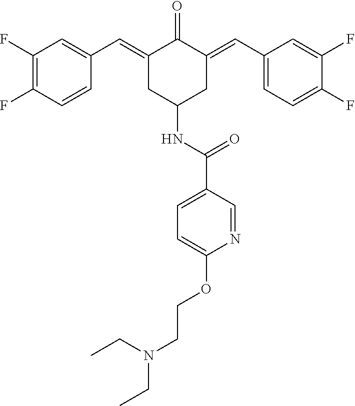 [SMILES: CCN(CC)CCOc1ccc(C(=O)NC2C/C(=C\c3ccc(F)c(F)c3)C(=O)/C(=C/c3ccc(F)c(F)c3)C2)cn1]

### N-(3,5-Bis((E)-3,4-difluorobenzylidene)-4-oxocyclohexyl)-6-(2-(diethylamino)ethoxy)nicotinamide (JCS115)

¹H NMR (500 MHz, CDCl₃) δ 8.10 (d, J=2.6 Hz, 1H), 7.84 (s, 2H), 7.55 (dd, J=9.5, 2.7 Hz, 1H), 7.33-7.20 (m, 6H), 6.50 (d, J=9.5 Hz, 1H), 6.16 (d, J=7.2 Hz, 1H), 4.53-4.44 (m, 1H), 4.00 (t, J=5.9 Hz, 2H), 3.30 (br d, J=14.8 Hz, 2H), 3.07 (dd, J=16.9, 8.2 Hz, 2H), 2.75 (t, J=5.9 Hz, 2H), 2.53 (q, J=7.1 Hz, 4H), 0.94 (t, J=7.1 Hz, 6H).

¹³C NMR (126 MHz, CDCl₃) δ 187.7, 163.9, 162.3, 150.8 (dd, J_{CF}=211.6 Hz, J_{C-CF}=12.6 Hz, 2C), 150.3 (dd, J_{CF}=207.9 Hz, J_{C-CF}=12.6 Hz, 2C), 142.5, 138.0 (2C), 136.2, 132.6 (2C), 132.2 (dd, J_{C-C-CF}=5.0 Hz, J_{C-C-CF}=3.7 Hz, 2C), 127.3 (dd, J_{C-C-CF}=7.5 Hz, J_{C-C-CF}=3.7 Hz, 2C), 119.2, 119.1 (d, J_{C-CF}=17.6 Hz, 2C), 117.8 (d, J_{C-CF}=17.6 Hz, 2C), 111.7, 51.4, 49.0, 47.5, 44.7, 33.8, 12.2.

¹⁹F NMR (376 MHz, CDCl₃) δ −131.4 (d, J_{FF}=18.8 Hz, 2F), −133.0 (d, J_{FF}=22.5 Hz, 2F).

HR-APCI m/z calcd for C₃₂H₃₂F₄N₃O₃ [M+H]=582.2402, found 582.2374.

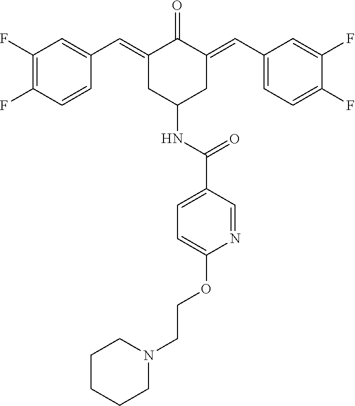 [SMILES: O=C1/C(=C/c2ccc(F)c(F)c2)CC(NC(=O)c2ccc(OCCN3CCCCC3)nc2)C/C1=C\c1ccc(F)c(F)c1]

### N-(3,5-Bis((E)-3,4-difluorobenzylidene)-4-oxocyclohexyl)-6-(2-(piperidin-1-yl)ethoxy)nicotinamide (JCS116)

¹H NMR (500 MHz, CDCl₃) δ 8.08 (s, 1H), 7.75 (s, 2H), 7.55 (d, J=9.5 Hz, 1H), 7.25-7.13 (m, 6H), 6.42-6.40 (m, 2H), 4.47-4.42 (m, 1H), 3.99 (t, J=6.1 Hz, 2H), 3.25 (br d, J=13.8 Hz, 2H), 3.00 (dd, J=14.7, 8.9 Hz, 2H), 2.55 (t, J=6.1 Hz, 2H), 2.36 (t, J=5.3 Hz, 4H), 1.48-1.44 (m, 4H), 1.37-1.34 (m, 2H).

¹³C NMR (126 MHz, CDCl₃) δ 187.6, 163.9, 162.2, 150.7 (dd, J_{CF}=254.5 Hz, J_{C-CF}=12.6 Hz, 2C), 150.2 (dd, J_{CF}=249.8 Hz, J_{C-CF}=12.6 Hz, 2C), 142.2, 137.8 (2C), 136.4, 132.7 (2C), 132.2 (dd, J_{C-C-CF}=5.0 Hz, J_{C-C-CF}=3.7 Hz, 2C), 127.3 (dd, J_{C-C-CF}=7.5 Hz, J_{C-C-CF}=3.7 Hz, 2C), 119.0, 118.9 (d, J_{C-CF}=17.6 Hz, 2C), 117.7 (d, J_{C-CF}=17.6 Hz, 2C), 112.1, 57.1, 54.6, 47.1, 44.8, 33.8, 26.1, 24.2.

¹⁹F NMR (376 MHz, CDCl₃) δ −134.8 (d, J_{FF}=18.8 Hz, 2F), −136.4 (d, J_{FF}=22.5 Hz, 2F).

HR-APCI m/z calcd for C₃₃H₃₂F₄N₃O₃ [M+H]=594.2405, found 594.2374.

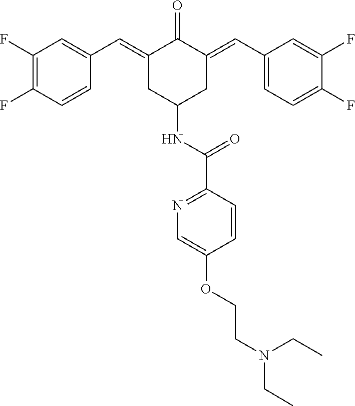 [SMILES: CCN(CC)CCOc1ccc(C(=O)NC2C/C(=C\c3ccc(F)c(F)c3)C(=O)/C(=C/c3ccc(F)c(F)c3)C2)nc1]

### N-(3,5-Bis((E)-3,4-difluorobenzylidene)-4-oxocyclohexyl)-5-(2-(diethylamino)ethoxy)picolinamide (JCS131)

¹H NMR (500 MHz, CDCl₃) δ 8.17 (d, J=2.8 Hz, 1H), 8.05 (d, J=8.7 Hz, 1H), 7.97 (d, J=8.0 Hz, 1H), 7.79 (s, 2H), 7.29-7.17 (m, 7H), 4.48-4.39 (m, 1H), 4.12 (t, J=6.0 Hz, 2H), 3.28 (br d, J=15.7 Hz, 2H), 3.02 (dd, J=16.9, 10.2 Hz, 2H), 2.89 (t, J=6.0 Hz, 2H), 2.64 (q, J=7.1 Hz, 4H), 1.07 (t, J=7.1 Hz, 6H).

¹³C NMR (126 MHz, CDCl₃) δ 188.0, 163.9, 157.5, 150.7 (dd, J_{CF}=254.5 Hz, J_{C-CF}=12.6 Hz, 2C), 150.2 (dd, J_{CF}=249.8 Hz, J_{C-CF}=12.6 Hz, 2C), 142.0, 137.6 (2C), 136.9, 133.1 (2C), 132.2 (dd, J_{C-C-CF}=5.0 Hz, J_{C-C-CF}=3.7 Hz, 2C), 127.3 (dd, J_{C-C-CF}=7.5 Hz, J_{C-C-CF}=3.7 Hz, 2C), 123.5, 120.9, 118.9 (d, J_{C-CF}=17.6 Hz, 2C), 117.7 (d, J_{C-CF}=17.6 Hz, 2C), 67.5, 51.6, 48.0, 44.4, 34.1, 11.8.

¹⁹F NMR (376 MHz, CDCl₃) δ −135.4 (d, J_{FF}=18.8 Hz, 2F), −136.6 (d, J_{FF}=22.5 Hz, 2F).

HR-APCI m/z calcd for C₃₂H₃₂F₄N₃O₃ [M+H]=582.2454, found 582.2374.

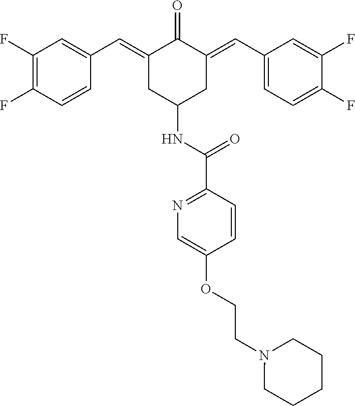 [SMILES: O=C1/C(=C/c2ccc(F)c(F)c2)CC(NC(=O)c2ccc(OCCN3CCCCC3)cn2)C/C1=C\c1ccc(F)c(F)c1]

### N-(3,5-Bis((E)-3,4-difluorobenzylidene)-4-oxocyclohexyl)-5-(2-(piperidin-1-yl)ethoxy)picolinamide (JCS132)

¹H NMR (500 MHz, CDCl₃) δ 8.17 (d, J=2.8 Hz, 1H), 8.04 (d, J=8.7 Hz, 1H), 7.97 (d, J=8.0 Hz, 1H), 7.78 (s, 2H), 7.28-7.14 (m, 7H), 4.45-4.40 (m, 1H), 4.16 (t, J=5.9 Hz, 2H), 3.27 (br d, J=15.8 Hz, 2H), 3.02 (dd, J=15.7, 11.0 Hz, 2H), 2.78 (t, J=5.9 Hz, 2H), 2.49 (t, J=5.4 Hz, 4H), 1.62-1.56 (m, 4H), 1.46-1.40 (m, 2H).

¹³C NMR (126 MHz, CDCl₃) δ 187.9, 163.9, 157.5, 150.7 (dd, J_{CF}=254.5 Hz, J_{C-CF}=12.6 Hz, 2C), 150.2 (dd, J_{CF}=249.8 Hz, J_{C-CF}=12.6 Hz, 2C), 142.0, 137.6 (2C), 136.9, 133.1 (2C), 132.3 (dd, J_{C-C-CF}=5.0 Hz, J_{C-C-CF}=3.7 Hz, 2C), 127.1 (dd, J_{C-C-CF}=7.5 Hz, J_{C-C-CF}=3.7 Hz, 2C), 123.5, 121.0, 119.0 (d, J_{C-CF}=17.6 Hz, 2C), 117.7 (d, J_{C-CF}=17.6 Hz, 2C), 66.8, 57.7, 55.2, 44.3, 34.1, 25.9, 24.1.

¹⁹F NMR (376 MHz, CDCl₃) δ −135.4 (d, J_{FF}=18.8 Hz, 2F), −136.6 (d, J_{FF}=22.5 Hz, 2F).

HR-APCI m/z calcd for C₃₃H₃₂F₄N₃O₃ [M+H]=594.2453, found 594.2374.

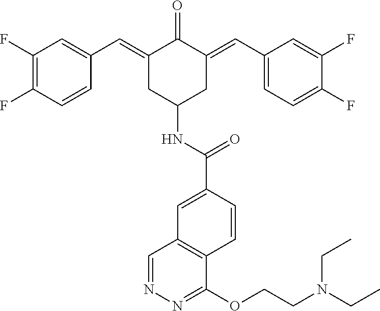 [SMILES: CCN(CC)CCOc1nncc2cc(C(=O)NC3C/C(=C\c4ccc(F)c(F)c4)C(=O)/C(=C/c4ccc(F)c(F)c4)C3)ccc12]

### N-(3,5-Bis((E)-3,4-difluorobenzylidene)-4-oxocyclohexyl)-1-(2-(diethylamino)ethoxy)phthalazine-6-carboxamide (JCS133)

¹H NMR (500 MHz, CDCl₃) δ 8.28 (d, J=8.5 Hz, 1H), 8.06 (s, 1H), 7.90 (s, 1H), 7.80 (s, 2H), 7.77 (d, J=8.1 Hz, 1H), 7.26-7.16 (m, 6H), 6.49 (d, J=7.2 Hz, 1H), 4.56-4.51 (m, 1H), 3.98 (t, J=5.7 Hz, 2H), 3.29 (br d, J=14.5 Hz, 2H), 3.10 (dd, J=15.7, 7.9 Hz, 2H), 2.72 (t, J=5.8 Hz, 2H), 2.50 (q, J=7.1 Hz, 4H), 0.88 (t, J=7.1 Hz, 6H).

¹³C NMR (126 MHz, CDCl₃) δ 187.6, 166.1, 160.5, 150.7 (dd, J_{CF}=254.5 Hz, J_{C-CF}=12.6 Hz, 2C), 150.2 (dd, J_{CF}=249.8 Hz, J_{C-CF}=12.6 Hz, 2C), 148.7, 148.2, 139.3, 138.2 (2C), 132.4 (2C), 132.2 (dd, J_{C-C-CF}=5.0 Hz, J_{C-C-CF}=3.7 Hz, 2C), 127.5, 127.2 (dd, J_{C-C-CF}=7.5 Hz, J_{C-C-CF}=3.7 Hz, 2C), 125.8, 125.3, 124.1, 119.0 (d, J_{C-CF}=17.6 Hz, 2C), 117.7 (d, J_{C-CF}=17.6 Hz, 2C), 51.3, 47.6, 45.7, 45.1, 33.6, 12.2.

¹⁹F NMR (376 MHz, CDCl₃) δ −134.9 (d, J_{FF}=18.8 Hz, 2F), −136.4 (d, J_{FF}=22.5 Hz, 2F).

HR-APCI m/z calcd for C₃₅H₃₃F₄N₄O₃ [M+H]=633.2575, found 633.2483.

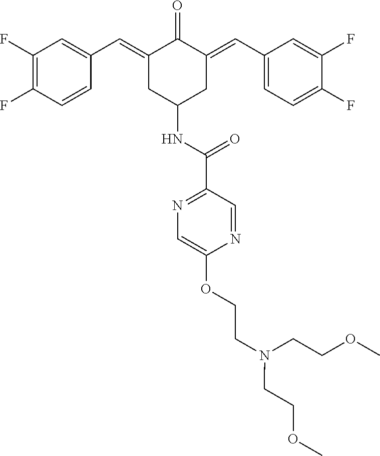 [SMILES: COCCN(CCOC)CCOc1cnc(C(=O)NC2C/C(=C\c3ccc(F)c(F)c3)C(=O)/C(=C/c3ccc(F)c(F)c3)C2)cn1]

### N-(3,5-Bis((E)-3,4-difluorobenzylidene)-4-oxocyclohexyl)-5-(2-(bis(2-methoxyethyl)amino)ethoxy)-pyrazine-2-carboxamide (JCS134)

¹H NMR (500 MHz, CDCl₃) δ 8.36 (s, 1H), 7.93 (s, 1H), 7.80 (s, 2H), 7.52 (d, J=7.9 Hz, 1H), 7.30-7.16 (m, 6H), 4.48-4.40 (m, 1H), 3.94 (t, J=5.6 Hz, 2H), 3.32-3.24 (m, 6H), 3.22 (s, 6H), 3.01 (dd, J=15.8, 9.1 Hz, 2H), 2.89 (t, J=5.7 Hz, 2H), 2.68 (t, J=5.3 Hz, 4H).

¹³C NMR (126 MHz, CDCl₃) δ 187.7, 162.5, 156.3, 150.7 (dd, J_{CF}=254.5 Hz, J_{C-CF}=12.6 Hz, 2C), 150.2 (dd, J_{CF}=249.8 Hz, J_{C-CF}=12.6 Hz, 2C), 145.2, 137.7 (2C), 134.0, 132.8 (2C), 132.2 (dd, J_{C-C-CF}=5.0 Hz, J_{C-C-CF}=3.7 Hz, 2C), 127.1 (dd, J_{C-C-CF}=7.5 Hz, J_{C-C-CF}=3.7 Hz, 2C), 125.4, 118.8 (d, J_{C-CF}=17.6 Hz, 2C), 117.6 (d, J_{C-CF}=17.6 Hz, 2C), 71.5, 58.6, 55.0, 53.4, 48.5, 44.1, 33.9.

¹⁹F NMR (376 MHz, CDCl₃) δ −135.2 (d, J_{FF}=18.8 Hz, 2F), −136.6 (d, J_{FF}=22.5 Hz, 2F).

HR-APCI m/z calcd for C₃₃H₃₅F₄N₄O₅ [M+H]=643.2587, found 643.2538.

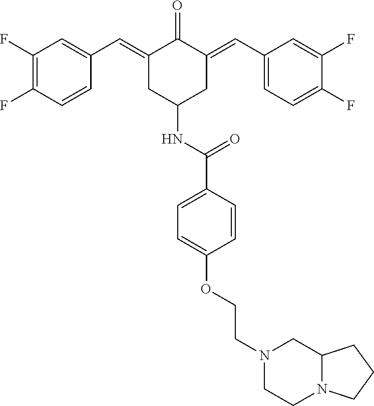 [SMILES: O=C1/C(=C/c2ccc(F)c(F)c2)CC(NC(=O)c2ccc(OCCN3CCN4CCCC4C3)cc2)C/C1=C\c1ccc(F)c(F)c1]

### N-(3,5-Bis((E)-3,4-difluorobenzylidene)-4-oxocyclohexyl)-4-(2-(hexahydropyrrolo[1,2-a]pyrazin-2(1H)-yl)ethoxy)benzamide (JCS135)

¹H NMR (500 MHz, CDCl₃) δ 7.80 (s, 2H), 7.62 (d, J=8.7 Hz, 2H), 7.29-7.16 (m, 6H), 6.89 (d, J=8.7 Hz, 2H), 6.05 (d, J=7.3 Hz, 1H), 4.53-4.46 (m, 1H), 4.10 (t, J=6.0 Hz, 2H), 3.25 (br d, J=14.2 Hz, 2H), 3.06 (dd, J=16.1, 8.0 Hz, 2H), 2.93 (t, J=6.0 Hz, 2H), 2.81 (t, J=6.0 Hz, 2H), 2.68-2.58 (m, 2H), 2.08-2.05 (m, 2H), 1.80-1.35 (m, 7H). ¹³C NMR (126 MHz, CDCl₃) δ 188.0, 166.6, 161.8, 150.7 (dd, J_{CF}=254.5 Hz, J_{C-CF}=12.6 Hz, 2C), 150.2 (dd, J_{CF}=249.8 Hz, J_{C-CF}=12.6 Hz, 2C), 137.7 (2C), 132.8 (2C), 132.2 (dd, J_{C-C-CF}=5.0 Hz, J_{C-C-CF}=3.7 Hz, 2C), 128.8 (2C), 127.1 (dd, J_{C-C-CF}=7.5 Hz, J_{C-C-CF}=3.7 Hz, 2C), 126.2, 119.0 (d, J_{C-CF}=17.6 Hz, 2C), 117.7 (d, J_{C-CF}=17.6 Hz, 2C), 114.5 (2C), 67.3, 62.3 (2C), 54.6, 44.6, 42.5 (2C), 33.8, 32.5 (2C's), 25.7; several upfield carbons are overlapping.

¹⁹F NMR (376 MHz, CDCl₃) δ −135.0 (d, J_{FF}=18.8 Hz, 2F), −136.5 (d, J_{FF}=22.5 Hz, 2F).

HR-APCI m/z calcd for C₃₆H₃₅F₄N₃O₃K [M+K]=672.2124, found 672.2246.

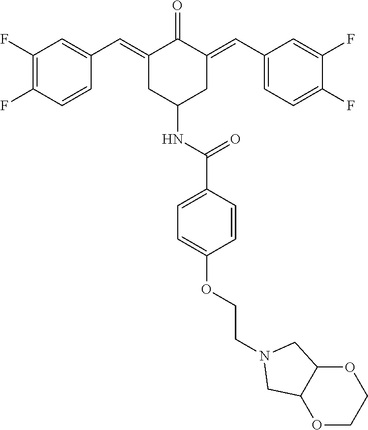 [SMILES: O=C1/C(=C/c2ccc(F)c(F)c2)CC(NC(=O)c2ccc(OCCN3CC4OCCOC4C3)cc2)C/C1=C\c1ccc(F)c(F)c1]

### N-(3,5-Bis((E)-3,4-difluorobenzylidene)-4-oxocyclohexyl)-4-(2-(hexahydro-6H-[1,4]dioxino[2,3-c]pyrrol-6-yl)ethoxy)benzamide (JCS137)

¹H NMR (500 MHz, CDCl₃) δ 7.80 (s, 2H), 7.62 (d, J=8.8 Hz, 2H), 7.27-7.17 (m, 6H), 6.87 (d, J=8.8 Hz, 2H), 6.10 (d, J=7.3 Hz, 1H), 4.52-4.46 (m, 1H), 4.10 (t, J=5.1 Hz, 4H), 3.84-3.80 (m, 2H), 3.60-3.55 (m, 2H), 3.25 (br d, J=15.2 Hz, 2H), 3.05 (dd, J=15.9, 8.2 Hz, 2H), 3.01-2.95 (m, 6H).

¹³C NMR (126 MHz, CDCl₃) δ 187.9, 166.6, 161.6, 150.7 (dd, J_{CF}=254.5 Hz, J_{C-CF}=12.6 Hz, 2C), 150.2 (dd, J_{CF}=249.8 Hz, J_{C-CF}=12.6 Hz, 2C), 137.9 (2C), 132.8 (2C), 132.2 (dd, J_{C-C-CF}=5.0 Hz, J_{C-C-CF}=3.7 Hz, 2C), 128.8 (2C), 127.1 (dd, J_{C-C-CF}=7.5 Hz, J_{C-C-CF}=3.7 Hz, 2C), 126.3, 119.0 (d, J_{C-CF}=17.6 Hz, 2C), 117.7 (d, J_{C-CF}=17.6 Hz, 2C), 114.4 (2C), 73.5, 67.3, 62.6, 55.8, 55.4, 44.6, 33.8.

¹⁹F NMR (376 MHz, CDCl₃) δ −135.1 (d, J_{FF}=18.8 Hz, 2F), −136.5 (d, J_{FF}=22.5 Hz, 2F).

HR-APCI m/z calcd for C₃₅H₃₃F₄N₂O₅ [M+H]=637.2332, found 637.2320.

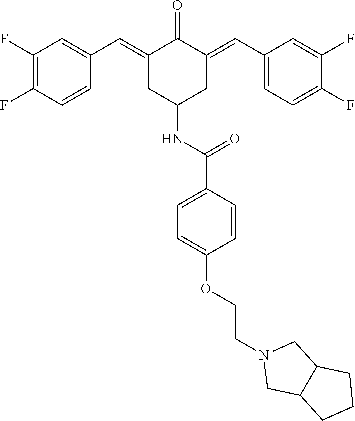 [SMILES: O=C1/C(=C/c2ccc(F)c(F)c2)CC(NC(=O)c2ccc(OCCN3CC4CCCC4C3)cc2)C/C1=C\c1ccc(F)c(F)c1]

### N-(3,5-Bis((E)-3,4-difluorobenzylidene)-4-oxocyclohexyl)-4-(2-(hexahydrocyclopenta[c]pyrrol-2(1H)-yl)ethoxy)benzamide (JCS138)

¹H NMR (500 MHz, CDCl₃) δ 7.82 (s, 2H), 7.66 (d, J=8.4 Hz, 2H), 7.34-7.16 (m, 6H), 6.91 (d, J=8.4 Hz, 2H), 6.19 (d, J=7.3 Hz, 1H), 4.52-4.46 (m, 1H), 4.15 (t, J=5.8 Hz, 2H), 3.28 (br d, J=12.8 Hz, 2H), 3.13-3.03 (m, 4H), 2.95-2.86 (m, 3H), 2.47-2.27 (m, 2H), 2.21-2.02 (m, 3H), 1.91-1.70 (m, 3H), 1.49-1.36 (m, 1H).

¹³C NMR (126 MHz, CDCl₃) δ 187.9, 166.6, 161.6, 150.7 (dd, J_{CF}=254.5 Hz, J_{C-CF}=12.6 Hz, 2C), 150.2 (dd, J_{CF}=249.8 Hz, J_{C-CF}=12.6 Hz, 2C), 137.9 (2C), 132.8 (2C), 132.2 (dd, J_{C-C-CF}=5.0 Hz, J_{C-C-CF}=3.7 Hz, 2C), 128.8 (2C), 127.1 (dd, J_{C-C-CF}=7.5 Hz, J_{C-C-CF}=3.7 Hz, 2C), 126.2, 119.0 (d, J_{C-CF}=17.6 Hz, 2C), 117.7 (d, J_{C-CF}=17.6 Hz, 2C), 114.5 (2C), 66.2, 62.6, 58.5, 53.4, 51.6, 44.6, 33.8, 27.7.

¹⁹F NMR (376 MHz, CDCl₃) δ −135.1 (d, J_{FF}=18.8 Hz, 2F), −136.5 (d, J_{FF}=22.5 Hz, 2F).

HR-APCI m/z calcd for C₃₆H₃₄F₄N₂O₃ [M+]=618.2512, found 618.2500.

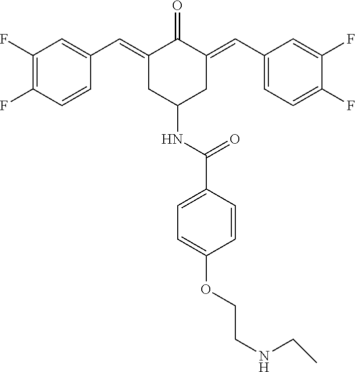 [SMILES: CCNCCOc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-(2-(ethylamino)ethoxy)benzamide (JCS140)

¹H NMR (500 MHz, CDCl₃) δ 7.77 (s, 2H), 7.61 (d, J=8.4 Hz, 2H), 7.26-7.16 (m, 6H), 6.85 (d, J=8.5 Hz, 2H), 6.33 (m, 1H), 4.49-4.42 (m, 1H), 4.10 (t, J=5.2 Hz, 2H), 3.24 (br d, J=15.2 Hz, 2H), 3.09-2.99 (m, 4H), 2.75 (q, J=7.2 Hz, 2H), 1.14 (t, J=7.1 Hz, 3H).

¹³C NMR (126 MHz, CDCl₃) δ 187.8, 166.6, 161.5, 150.7 (dd, J_{CF}=254.5 Hz, J_{C-CF}=12.6 Hz, 2C), 150.2 (dd, J_{CF}=249.8 Hz, J_{C-CF}=12.6 Hz, 2C), 137.7 (2C), 132.7 (2C), 132.1 (dd, J_{C-C-CF}=5.0 Hz, J_{C-C-CF}=3.7 Hz, 2C), 128.7 (2C), 127.1 (dd, J_{C-C-CF}=7.5 Hz, J_{C-C-CF}=3.7 Hz, 2C), 126.3, 118.9 (d, J_{C-CF}=17.6 Hz, 2C), 117.6 (d, J_{C-CF}=17.6 Hz, 2C), 114.2 (2C), 67.0, 47.9, 44.6, 43.8, 33.7, 14.6.

¹⁹F NMR (376 MHz, CDCl₃) δ −131.2 (d, J_{FF}=18.8 Hz, 2F), −132.6 (d, J_{FF}=22.5 Hz, 2F).

HR-APCI m/z calcd for C₃₁H₂₉F₄N₂O₃ [M+H]=553.2103, found 553.2108.

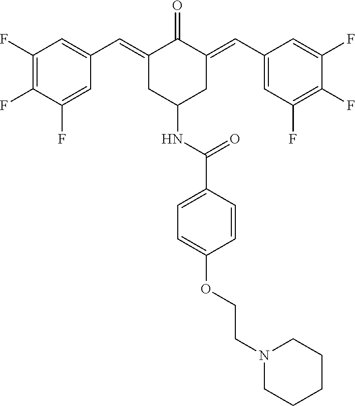 [SMILES: O=C1/C(=C/c2cc(F)c(F)c(F)c2)CC(NC(=O)c2ccc(OCCN3CCCCC3)cc2)C/C1=C\c1cc(F)c(F)c(F)c1]

### N-(4-Oxo-3,5-bis((E)-3,4,5-trifluorobenzylidene)cyclohexyl)-4-(2-(piperidin-1-yl)ethoxy)benzamide (JCS141)

¹H NMR (500 MHz, CDCl₃) δ 7.71 (s, 2H), 7.68 (d, J=8.9 Hz, 2H), 7.14-6.98 (m, 4H), 6.89 (d, J=8.9 Hz, 2H), 6.45 (d, J=7.2 Hz, 1H), 4.52-4.42 (m, 1H), 4.18 (t, J=5.9 Hz, 2H), 3.27 (br d, J=13.1 Hz, 2H), 3.06 (dd, J=17.2, 9.6 Hz, 2H), 2.87 (t, J=5.9 Hz, 2H), 2.62 (t, J=5.3 Hz, 4H), 1.71-1.63 (m, 4H), 1.53-1.45 (m, 2H).

¹³C NMR (126 MHz, CDCl₃) δ 187.4, 166.7, 161.6, 151.2 (dd, J_{C-F}=210.4, 3.7 Hz, 2C), 151.1 (dd, J_{C-F}=209.1, 3.7 Hz, 2C), 140.0 (dt, J_{C-F}=214.2, 3.7 Hz, 2C), 136.8 (2C), 133.9 (2C), 128.9 (2C), 126.2, 114.5 (dd, J_{C-F}=13.8, 3.7 Hz, 2C), 114.4 (2C), 65.8, 57.6, 55.1, 44.7, 33.7, 25.6, 23.9.

¹⁹F NMR (376 MHz, CDCl₃) δ −133.3 (d, J_{FF}=26.3 Hz, 3F).

HR-APCI m/z calcd for C₃₄H₃₁F₆N₂O₃ [M+H]=629.2234, found 629.2233.

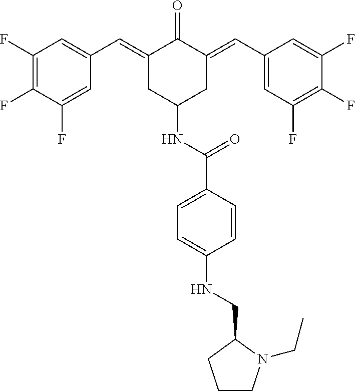 [SMILES: CCN1CCC[C@H]1CNc1ccc(C(=O)NC2C/C(=C\c3cc(F)c(F)c(F)c3)C(=O)/C(=C/c3cc(F)c(F)c(F)c3)C2)cc1]

### 4-((((S)-1-Ethylpyrrolidin-2-yl)methyl)amino)-N-(4-oxo-3,5-bis((E)-3,4,5-trifluorobenzylidene)-cyclohexyl)benzamide (JCS142)

¹H NMR (500 MHz, CDCl₃) δ 7.73 (s, 2H), 7.57 (d, J=8.7 Hz, 2H), 7.11-7.06 (m, 4H), 6.58 (d, J=8.7 Hz, 2H), 6.09 (d, J=7.3 Hz, 1H), 4.97 (m, 1H), 4.52-4.42 (m, 1H), 3.36-3.16 (m, 5H), 3.06 (dd, J=15.0, 7.6 Hz, 2H), 3.00-2.73 (m, 2H), 2.38-2.26 (m, 2H), 2.02-1.71 (m, 4H), 1.14 (t, J=7.2 Hz, 3H).

¹³C NMR (126 MHz, CDCl₃) δ 187.5, 166.9, 151.6, 151.2 (dd, J_{C-F}=210.4, 3.7 Hz, 2C), 151.1 (dd, J_{C-F}=209.1, 3.7 Hz, 2C), 140.0 (dt, J_{C-F}=214.2, 3.7 Hz, 2C), 136.8 (2C), 133.9 (2C), 128.6 (2C), 121.2, 114.3 (dd, J_{C-F}=13.8, 3.7 Hz, 2C), 111.7 (2C), 62.8, 53.6, 48.4, 44.3, 44.2, 33.7, 28.5, 22.7, 13.5.

¹⁹F NMR (376 MHz, CDCl₃) δ −133.2 (d, J_{FF}=26.3 Hz, 3F).

HR-APCI m/z calcd for C₃₄H₃₂F₆N₃O₂ [M+H]=628.2391, found 628.2393.

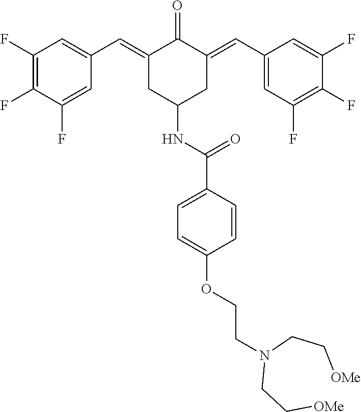 [SMILES: COCCN(CCOC)CCOc1ccc(C(=O)NC2C/C(=C\c3cc(F)c(F)c(F)c3)C(=O)/C(=C/c3cc(F)c(F)c(F)c3)C2)cc1]

### 4-(2-(Bis(2-methoxyethyl)amino)ethoxy)-N-(4-oxo-3,5-bis((E)-3,4,5-trifluorobenzylidene)cyclo-hexyl)benzamide (JCS143)

¹H NMR (500 MHz, CDCl₃) δ 7.74 (s, 2H), 7.67 (d, J=8.8 Hz, 2H), 7.14-7.04 (m, 4H), 6.92 (d, J=8.8 Hz, 2H), 6.18 (d, J=7.2 Hz, 1H), 4.52-4.42 (m, 1H), 4.12 (t, J=6.1 Hz, 2H), 3.52 (t, J=5.8 Hz, 4H), 3.37 (s, 6H), 3.29 (br d, J=15.6 Hz, 2H), 3.14-3.02 (m, 4H), 2.88 (t, J=5.8 Hz, 4H).

¹³C NMR (126 MHz, CDCl₃) δ 187.4, 166.7, 161.8, 151.2 (dd, J_{C-F}=210.4, 3.7 Hz, 2C), 151.1 (dd, J_{C-F}=209.1, 3.7 Hz, 2C), 140.0 (dt, J_{C-F}=214.2, 3.7 Hz, 2C), 137.0 (2C), 133.7 (2C), 128.8 (2C), 126.0, 114.5 (2C), 114.4 (dd, J_{C-F}=13.8, 3.7 Hz, 2C), 71.2, 66.8, 58.9, 54.8, 53.9, 44.6, 33.7.

¹⁹F NMR (376 MHz, CDCl₃) δ −133.3 (d, J_{FF}=26.3 Hz, 3F).

HR-APCI m/z calcd for C₃₅H₃₅F₆N₂O₅ [M+H]=677.2445, found 677.2444.

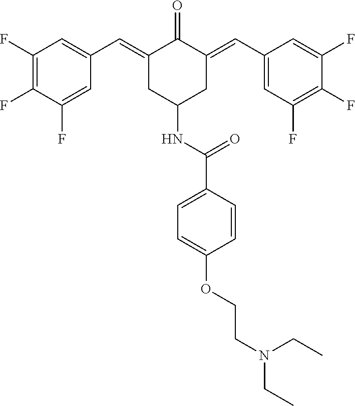 [SMILES: CCN(CC)CCOc1ccc(C(=O)NC2C/C(=C\c3cc(F)c(F)c(F)c3)C(=O)/C(=C/c3cc(F)c(F)c(F)c3)C2)cc1]

### 4-(2-(Diethylamino)ethoxy)-N-(4-oxo-3,5-bis((E)-3,4,5-trifluorobenzylidene)cyclohexyl)benzamide (JCS144)

¹H NMR (500 MHz, CDCl₃) δ 7.71 (s, 2H), 7.63 (d, J=8.8 Hz, 2H), 7.11-6.99 (m, 4H), 6.88 (d, J=8.8 Hz, 2H), 6.15 (d, J=7.2 Hz, 1H), 4.52-4.42 (m, 1H), 4.10 (t, J=6.1 Hz, 2H), 3.25 (br d, J=13.7 Hz, 2H), 3.03 (dd, J=17.8, 8.6 Hz, 2H), 2.93 (t, J=6.1 Hz, 2H), 2.69 (q, J=7.1 Hz, 4H), 1.09 (t, J=7.2 Hz, 6H).

¹³C NMR (126 MHz, CDCl₃) δ 187.4, 166.7, 161.7, 151.2 (dd, J_{C-F}=210.4, 3.7 Hz, 2C), 151.1 (dd, J_{C-F}=209.1, 3.7 Hz, 2C), 140.0 (dt, J_{C-F}=214.2, 3.7 Hz, 2C), 137.0 (2C), 133.8 (2C), 128.8 (2C), 126.1, 114.5 (2C), 114.4 (dd, J_{C-F}=13.8, 3.7 Hz, 2C), 66.6, 51.5, 47.8, 44.6, 33.8, 11.5.

¹⁹F NMR (376 MHz, CDCl₃) δ −133.1 (d, J_{FF}=26.3 Hz, 3F).

HR-APCI m/z calcd for C₃₃H₃₁F₆N₂O₃ [M+H]=617.2233, found 617.2233.

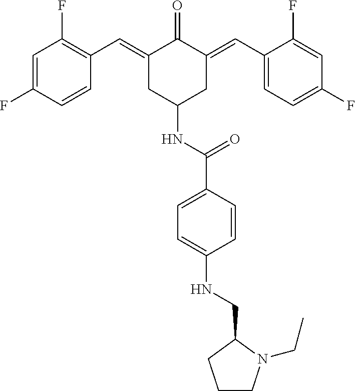 [SMILES: CCN1CCC[C@H]1CNc1ccc(C(=O)NC2C/C(=C\c3ccc(F)cc3F)C(=O)/C(=C/c3ccc(F)cc3F)C2)cc1]

### N-(3,5-Bis((E)-2,4-difluorobenzylidene)-4-oxocyclohexyl)-4-((((S)-1-ethylpyrrolidin-2-yl)methyl)-amino)benzamide (JCS145)

¹H NMR (500 MHz, CDCl₃) δ 7.88 (s, 2H), 7.50 (d, J=8.7 Hz, 2H), 7.37-7.31 (m, 2H), 6.95-6.80 (m, 4H), 6.53 (d, J=8.8 Hz, 2H), 6.01 (d, J=7.5 Hz, 1H), 4.97 (br s, 1H), 4.52-4.42 (m, 1H), 3.28-3.07 (m, 5H), 2.96 (dd, J=15.9, 7.6 Hz, 2H), 2.88-2.69 (m, 2H), 2.32-2.18 (m, 2H), 1.96-1.68 (m, 4H), 1.10 (t, J=7.2 Hz, 3H).

¹³C NMR (126 MHz, CDCl₃) δ 187.9, 166.9, 163.4 (dd, J_{C-F}=277.2, 10.4 Hz, 2C), 161.5 (dd, J_{C-F}=278.4, 11.3 Hz, 2C), 151.6, 134.5 (2C), 131.9 (2C), 131.6 (dd, J_{C-F}=7.5, 3.7 Hz, 2C), 128.7 (2C), 121.5, 119.6 (dd, J_{C-F}=11.3, 2.5 Hz, 2C), 111.7 (2C), 111.6 (dd, J_{C-F}=17.6, 6.3 Hz, 2C), 104.5 (t, J_{C-F}=21.4 Hz, 2C), 62.9, 53.6, 48.5, 44.4, 44.4, 34.2, 28.6, 22.8, 13.5.

¹⁹F NMR (376 MHz, CDCl₃) δ −106.8 (d, J_{FF}=7.5 Hz, 2F), −107.1 (d, J_{FF}=7.5 Hz, 2F).

HR-APCI m/z calcd for C₃₄H₃₄F₄N₃O₂ [M+H]=592.2583, found 592.2581.

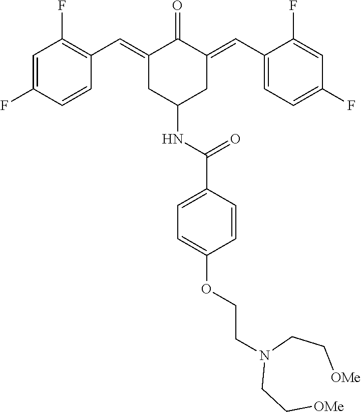 [SMILES: COCCN(CCOC)CCOc1ccc(C(=O)NC2C/C(=C\c3ccc(F)cc3F)C(=O)/C(=C/c3ccc(F)cc3F)C2)cc1]

### N-(3,5-Bis((E)-2,4-difluorobenzylidene)-4-oxocyclohexyl)-4-(2-(bis(2-methoxyethyl)amino)ethoxy)-benzamide (JCS146)

¹H NMR (500 MHz, CDCl₃) δ 7.80 (s, 2H), 7.60 (d, J=8.8 Hz, 2H), 7.30-7.24 (m, 2H), 6.86-6.44 (m, 6H), 6.45 (d, J=7.5 Hz, 1H), 4.46-4.38 (m, 1H), 4.02 (t, J=6.1 Hz, 2H), 3.44 (t, J=5.8 Hz, 4H), 3.29 (s, 6H), 3.07 (br d, J=14.6 Hz, 2H), 2.98 (t, J=6.1 Hz, 2H), 2.92 (dd, J=15.6, 8.6 Hz, 2H), 2.80 (t, J=5.8 Hz, 4H).

¹³C NMR (126 MHz, CDCl₃) δ 187.5, 166.5, 163.0 (dd, J_{C-F}=277.2, 10.4 Hz, 2C), 161.5, 161.3 (dd, J_{C-F}=278.4, 11.3 Hz, 2C), 134.3 (2C), 131.6 (2C), 131.5 (dd, J_{C-F}=7.5, 3.7 Hz, 2C), 128.7 (2C), 126.2, 119.4 (dd, J_{C-F}=11.3, 2.5 Hz, 2C), 114.2 (2C), 111.5 (dd, J_{C-F}=17.6, 6.3 Hz, 2C), 104.4 (t, J_{C-F}=21.4 Hz, 2C), 71.1, 66.7, 58.8, 54.6, 53.7, 44.6, 33.9.

¹⁹F NMR (376 MHz, CDCl₃) δ −106.8 (d, J_{FF}=11.2 Hz, 2F), −106.9 (d, J_{FF}=11.2 Hz, 2F).

HR-APCI m/z calcd for C₃₅H₃₇F₄N₂O₅ [M+H]=641.2635, found 641.2633.

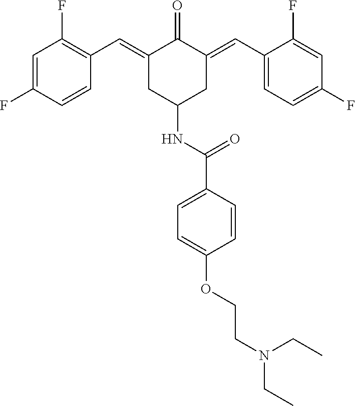 [SMILES: CCN(CC)CCOc1ccc(C(=O)NC2C/C(=C\c3ccc(F)cc3F)C(=O)/C(=C/c3ccc(F)cc3F)C2)cc1]

### N-(3,5-Bis((E)-2,4-difluorobenzylidene)-4-oxocyclohexyl)-4-(2-(diethylamino)ethoxy)benzamide (JCS147)

¹H NMR (500 MHz, CDCl₃) δ 7.88 (s, 2H), 7.61 (d, J=8.8 Hz, 2H), 7.34-7.30 (m, 2H), 6.96-6.81 (m, 6H), 6.15 (d, J=7.4 Hz, 1H), 4.52-4.45 (m, 1H), 4.09 (t, J=6.1 Hz, 2H), 3.11 (br d, J=14.1 Hz, 2H), 2.97 (dd, J=15.6, 8.3 Hz, 2H), 2.91 (t, J=6.1 Hz, 2H), 2.68 (q, J=7.1 Hz, 4H), 1.09 (t, J=7.2 Hz, 6H).

¹³C NMR (126 MHz, CDCl₃) δ 187.6, 166.4, 163.0 (dd, J_{C-F}=277.2, 10.4 Hz, 2C), 161.5, 161.3 (dd, J_{C-F}=278.4, 11.3 Hz, 2C), 134.2 (2C), 132.0 (2C), 131.5 (dd, J_{C-F}=7.5, 3.7 Hz, 2C), 128.6 (2C), 126.3, 119.4 (dd, J_{C-F}=11.3, 2.5 Hz, 2C), 114.3 (2C), 111.5 (dd, J_{C-F}=17.6, 6.3 Hz, 2C), 104.4 (t, J_{C-F}=21.4 Hz, 2C), 66.3, 51.4, 47.7, 44.5, 33.9, 11.4.

¹⁹F NMR (376 MHz, CDCl₃) δ −106.8 (d, J_{FF}=11.2 Hz, 2F), −106.9 (d, J_{FF}=11.2 Hz, 2F).

HR-APCI m/z calcd for C₃₃H₃₃F₄N₂O₃ [M+H]=581.2426, found 581.2421.

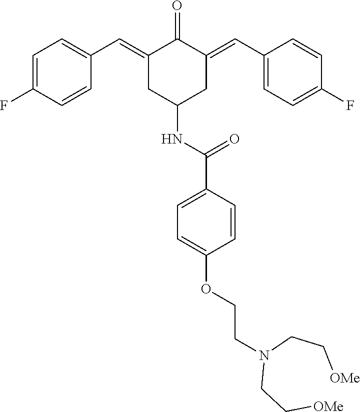 [SMILES: COCCN(CCOC)CCOc1ccc(C(=O)NC2C/C(=C\c3ccc(F)cc3)C(=O)/C(=C/c3ccc(F)cc3)C2)cc1]

### N-(3,5-Bis((E)-4-fluorobenzylidene)-4-oxocyclohexyl)-4-(2-(bis(2-methoxyethyl)amino)ethoxy)-benzamide (JCS148)

¹H NMR (500 MHz, CDCl₃) δ 7.85 (s, 2H), 7.61 (d, J=8.8 Hz, 2H), 7.40 (dd, J=8.7, 5.5 Hz, 4H), 7.06 (dd, J=8.7, 5.5 Hz, 4H), 6.85 (d, J=8.8 Hz, 2H), 6.24 (d, J=7.4 Hz, 1H), 4.52-4.47 (m, 1H), 4.05 (t, J=6.1 Hz, 2H), 3.47 (t, J=5.8 Hz, 4H), 3.32 (s, 6H), 3.24 (br d, J=13.9 Hz, 2H), 3.07 (dd, J=16.5, 7.5 Hz, 2H), 3.00 (t, J=6.1 Hz, 2H), 2.82 (t, J=5.8 Hz, 4H).

¹³C NMR (126 MHz, CDCl₃) δ 188.4, 166.6, 162.9 (d, J_{C-F}=250.7 Hz, 2C), 161.7, 138.8 (2C), 132.4 (d, J_{C-F}=7.5 Hz, 4C), 131.9 (2C), 131.4 (d, J_{C-F}=3.7 Hz, 2C), 128.7 (2C), 126.3, 115.8 (d, J_{C-F}=21.4 Hz, 4C), 114.3 (2C), 71.2, 66.8, 58.9, 54.7, 53.8, 44.6, 33.8.

¹⁹F NMR (376 MHz, CDCl₃) δ −110.5 (s, 2F)

HR-APCI m/z calcd for C₃₅H₃₉F₂N₂O₅ [M+H]=605.2824, found 605.2821.

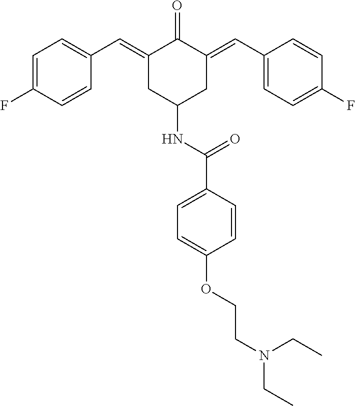 [SMILES: CCN(CC)CCOc1ccc(C(=O)NC2C/C(=C\c3ccc(F)cc3)C(=O)/C(=C/c3ccc(F)cc3)C2)cc1]

### N-(3,5-Bis((E)-4-fluorobenzylidene)-4-oxocyclohexyl)-4-(2-(diethylamino)ethoxy)benzamide (JCS149)

¹H NMR (500 MHz, CDCl₃) δ 7.87 (s, 2H), 7.61 (d, J=8.7 Hz, 2H), 7.41 (dd, J=8.6, 5.6 Hz, 4H), 7.07 (dd, J=8.6, 5.6 Hz, 4H), 6.86 (d, J=8.8 Hz, 2H), 6.18 (d, J=7.3 Hz, 1H), 4.52-4.47 (m, 1H), 4.04 (t, J=6.2 Hz, 2H), 3.24 (br d, J=14.1 Hz, 2H), 3.08 (dd, J=16.2, 7.6 Hz, 2H), 2.85 (t, J=6.2 Hz, 2H), 2.62 (q, J=7.1 Hz, 4H), 1.05 (t, J=7.1 Hz, 6H).

¹³C NMR (126 MHz, CDCl₃) δ 188.5, 166.6, 163.0 (d, J_{C-F}=252.0 Hz, 2C), 161.7, 138.9 (2C), 132.4 (d, J_{C-F}=7.5 Hz, 4C), 131.9 (2C), 131.4 (d, J_{C-F}=3.7 Hz, 2C), 128.7 (2C), 126.3, 115.8 (d, J_{C-F}=21.4 Hz, 4C), 114.3 (2C), 66.9, 51.6, 47.9, 44.6, 33.8, 11.9.

¹⁹F NMR (376 MHz, CDCl₃) δ −110.5 (s, 2F)

HR-APCI m/z calcd for C₃₃H₃₅F₂N₂O₃ [M+H]=545.2614, found 545.2610.

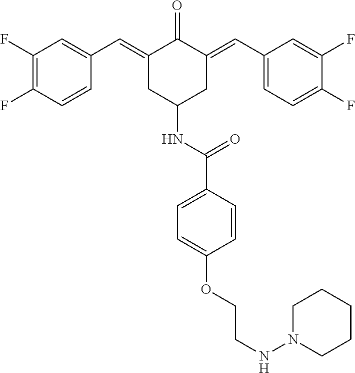 [SMILES: O=C1/C(=C/c2ccc(F)c(F)c2)CC(NC(=O)c2ccc(OCCNN3CCCCC3)cc2)C/C1=C\c1ccc(F)c(F)c1]

### N-(3,5-Bis((E)-3,4-difluorobenzylidene)-4-oxocyclohexyl)-4-(2-(piperidin-1-ylamino)ethoxy)-benzamide (JCS150)

¹H NMR (500 MHz, CDCl₃) δ 7.81 (s, 2H), 7.62 (d, J=8.8 Hz, 2H), 7.26-7.16 (m, 6H), 6.86 (d, J=8.8 Hz, 2H), 6.10 (d, J=7.3 Hz, 1H), 4.53-4.46 (m, 1H), 4.19 (t, J=5.8 Hz, 2H), 3.26 (br d, J=13.9 Hz, 2H), 3.06 (dd, J=16.8, 9.0 Hz, 2H), 2.90 (t, J=5.7 Hz, 2H), 2.64 (t, J=6.0 Hz, 4H), 1.71-1.63 (m, 4H), 1.51-1.44 (m, 2H).

¹³C NMR (126 MHz, CDCl₃) δ 188.0, 166.6, 161.4, 150.7 (dd, J_{CF}=254.5 Hz, J_{C-CF}=12.6 Hz, 2C), 150.2 (dd, J_{CF}=249.8 Hz, J_{C-CF}=12.6 Hz, 2C), 138.0 (2C), 132.8 (2C), 132.2 (dd, J_{C-C-CF}=5.0 Hz, J_{C-C-CF}=3.7 Hz, 2C), 128.8 (2C), 127.1 (dd, J_{C-C-CF}=7.5 Hz, J_{C-C-CF}=3.7 Hz, 2C), 126.5, 119.1 (d, J_{C-CF}=17.6 Hz, 2C), 117.7 (d, J_{C-CF}=17.6 Hz, 2C), 114.5 (2C), 65.7, 57.5, 55.0, 44.6, 33.9, 25.4, 23.8.

¹⁹F NMR (376 MHz, CDCl₃) δ −131.1 (d, J_{FF}=18.8 Hz, 2F), −132.6 (d, J_{FF}=22.5 Hz, 2F).

HR-APCI m/z calcd for C₃₄H₃₄F₄N₃O₃ [M+H]=608.2558, found 608.2530.

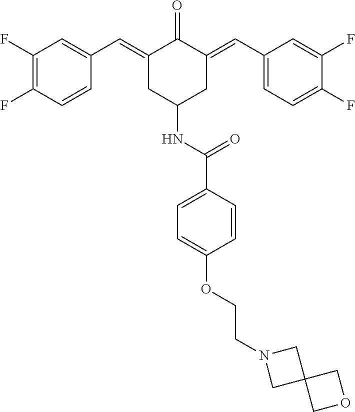 [SMILES: O=C1/C(=C/c2ccc(F)c(F)c2)CC(NC(=O)c2ccc(OCCN3CC4(COC4)C3)cc2)C/C1=C\c1ccc(F)c(F)c1]

### 4-(2-(2-Oxa-6-azaspiro[3.3]heptan-6-yl)ethoxy)-N-(3,5-bis((E)-3,4-difluorobenzylidene)-4-oxocyclohexyl)benzamide (JCS151)

¹H NMR (500 MHz, CDCl₃) δ 7.83 (s, 2H), 7.66 (d, J=8.8 Hz, 2H), 7.30-7.20 (m, 6H), 6.89 (d, J=8.8 Hz, 2H), 6.25 (d, J=7.3 Hz, 1H), 4.56-4.48 (m, 1H), 4.07 (t, J=5.3 Hz, 2H), 3.87 (s, 2H), 3.81 (s, 2H), 3.46 (br d, J=15.8 Hz, 2H), 3.38-3.26 (m, 4H), 3.10 (dd, J=16.6, 7.1 Hz, 2H), 2.99 (t, J=5.3 Hz, 2H).

¹³C NMR (126 MHz, CDCl₃) δ 187.8, 166.5, 161.3, 150.7 (dd, J_{CF}=254.5 Hz, J_{C-CF}=12.6 Hz, 2C), 150.2 (dd, J_{CF}=249.8 Hz, J_{C-CF}=12.6 Hz, 2C), 137.8 (2C), 132.7 (2C), 132.1 (dd, J_{C-C-CF}=5.0 Hz, J_{C-C-CF}=3.7 Hz, 2C), 128.7 (2C), 127.1 (dd, J_{C-C-CF}=7.5 Hz, J_{C-C-CF}=3.7 Hz, 2C), 126.4, 118.9 (d, J_{C-CF}=17.6 Hz, 2C), 117.6 (d, J_{C-CF}=17.6 Hz, 2C), 114.2 (2C), 66.5, 64.5, 56.7, 48.0, 44.6, 38.6, 33.7.

¹⁹F NMR (376 MHz, CDCl₃) δ −131.2 (d, J_{FF}=18.8 Hz, 2F), −132.6 (d, J_{FF}=22.5 Hz, 2F).

HR-APCI m/z calcd for C₃₄H₃₁F₄N₂O₄ [M+H]=607.2218, found 607.2214.

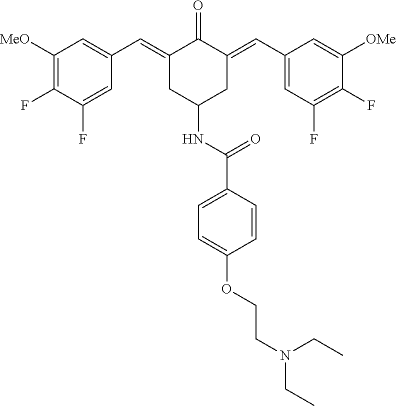 [SMILES: CCN(CC)CCOc1ccc(C(=O)NC2C/C(=C\c3cc(F)c(F)c(OC)c3)C(=O)/C(=C/c3cc(F)c(F)c(OC)c3)C2)cc1]

### N-(3,5-Bis((E)-3,4-difluoro-5-methoxybenzylidene)-4-oxocyclohexyl)-4-(2-(diethylamino)-ethoxy)benzamide (JCS152)

¹H NMR (500 MHz, CDCl₃) δ 7.77 (s, 2H), 7.63 (d, J=8.8 Hz, 2H), 6.89-6.85 (m, 4H), 6.81 (d, J=7.2 Hz, 2H), 6.13 (d, J=7.2 Hz, 1H), 4.52-4.48 (m, 1H), 4.14 (t, J=5.7 Hz, 2H), 3.91 (s, 6H), 3.25 (br d, J=14.1 Hz, 2H), 3.09 (dd, J=14.1, 7.2 Hz, 2H), 2.96 (t, J=6.0 Hz, 2H), 2.75 (q, J=7.1 Hz, 4H), 1.12 (t, J=7.1 Hz, 6H).

¹³C NMR (126 MHz, CDCl₃) δ 187.8, 166.5, 161.4, 150.9 (dd, J_{C-F}=248.2, 11.3 Hz, 2C), 149.1 (dd, J_{C-F}=8.8, 3.7 Hz, 2C), 141.4 (dd, J_{C-F}=254.5, 15.1 Hz, 2C), 138.4 (2C), 132.2 (2C), 130.6 (dd, J_{C-F}=8.8, 5.0 Hz, 2C), 128.7 (2C), 126.3, 114.3 (2C), 111.1 (d, J_{C-F}=2.5 Hz, 2C), 110.6 (dd, J_{C-F}=18.9 Hz, 2C), 56.8, 51.4, 47.7, 44.5, 33.6, 11.7.

¹⁹F NMR (376 MHz, CDCl₃) δ −136.2 (d, J_{FF}=18.8 Hz, 2F), −156.7 (d, J_{FF}=22.5 Hz, 2F).

HR-APCI m/z calcd for C₃₅H₃₇F₄N₂O₅ [M+H]=641.2633, found 641.2633.

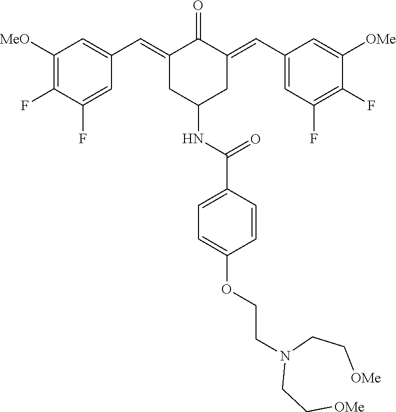 [SMILES: COCCN(CCOC)CCOc1ccc(C(=O)NC2C/C(=C\c3cc(F)c(F)c(OC)c3)C(=O)/C(=C/c3cc(F)c(F)c(OC)c3)C2)cc1]

### N-(3,5-Bis((E)-3,4-difluoro-5-methoxybenzylidene)-4-oxocyclohexyl)-4-(2-(bis(2-methoxyethyl)ami-no)ethoxy)benzamide (JCS153)

¹H NMR (500 MHz, CDCl₃) δ 7.75 (s, 2H), 7.62 (d, J=8.9 Hz, 2H), 6.92-6.84 (m, 4H), 6.79 (d, J=7.0 Hz, 2H), 6.19 (d, J=7.3 Hz, 1H), 4.52-4.48 (m, 1H), 4.08 (t, J=6.0 Hz, 2H), 3.90 (s, 6H), 3.49 (t, J=5.8 Hz, 4H), 3.32 (s, 6H), 3.25 (br d, J=15.6 Hz, 2H), 3.13-2.99 (m, 4H), 2.86 (t, J=5.7 Hz, 4H).

¹³C NMR (126 MHz, CDCl₃) δ 187.9, 166.7, 161.7, 150.9 (dd, J_{C-F}=248.2, 11.3 Hz, 2C), 149.2 (dd, J_{C-F}=8.8, 3.7 Hz, 2C), 141.4 (dd, J_{C-F}=254.5, 15.1 Hz, 2C), 138.4 (2C), 132.9 (2C), 130.7 (dd, J_{C-F}=8.8, 5.0 Hz, 2C), 128.7 (2C), 126.2, 114.4 (2C), 111.1 (d, J_{C-F}=2.5 Hz, 2C), 110.6 (dd, J_{C-F}=18.9 Hz, 2C), 71.0, 66.7, 58.9, 56.9, 54.8, 53.9, 44.7, 33.7.

¹⁹F NMR (376 MHz, CDCl₃) δ −136.2 (d, J_{FF}=18.8 Hz, 2F), −156.7 (d, J_{FF}=22.5 Hz, 2F).

HR-APCI m/z calcd for C₃₇H₄₁F₄N207 [M+H]=701.2840, found 701.2844.

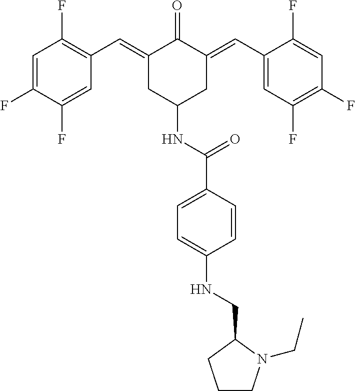 [SMILES: CCN1CCC[C@H]1CNc1ccc(C(=O)NC2C/C(=C\c3cc(F)c(F)cc3F)C(=O)/C(=C/c3cc(F)c(F)cc3F)C2)cc1]

### 4-((((S)-1-Ethylpyrrolidin-2-yl)methyl)amino)-N-(4-oxo-3,5-bis((E)-2,4,5-trifluorobenzylidene)-cyclohexyl)benzamide (JCS154)

¹H NMR (500 MHz, CDCl₃) δ 7.81 (s, 2H), 7.51 (d, J=8.7 Hz, 2H), 7.22-7.13 (m, 2H), 7.02-6.94 (m, 2H), 6.54 (d, J=8.7 Hz, 2H), 5.98 (d, J=8.7 Hz, 1H), 4.87 (br s, 1H), 4.52-4.42 (m, 1H), 3.32-3.07 (m, 5H), 3.02-2.67 (m, 4H), 2.30-2.19 (m, 2H), 1.99-1.64 (m, 4H), 1.09 (t, J=7.2 Hz, 3H).

¹³C NMR (126 MHz, CDCl₃) δ 166.9, 157.3, 156.3 (dd, J_{C-F}=252.0, 8.8 Hz, 2C), 151.8, 150.6 (dt, J_{C-F}=254.5, 12.6 Hz, 2C), 146.7 (dd, J_{C-F}=243.1, 11.3 Hz, 2C), 135.2 (2C), 131.0 (2C), 128.7 (2C), 121.3, 119.6 (dd, J_{C-F}=15.1, 5.0 Hz, 2C), 118.2 (dd, J_{C-F}=20.1, 3.7 Hz, 2C), 111.8 (2C), 106.2 (dd, J_{C-F}=27.7, 3.7 Hz, 2C), 62.8, 53.7, 44.4, 44.3, 34.2, 34.2, 28.6, 22.8, 13.7.

¹⁹F NMR (376 MHz, CDCl₃) δ −112.4 (d, J_{FF}=18.8, 11.2 Hz, 2F), −130.3 (d, J_{FF}=30.0, 7.5 Hz, 2F), −141.5 (d, J_{FF}=30.0, 18.8 Hz, 2F).

HR-APCI m/z calcd for C₃₄H₃₂F₆N₃O₂ [M+H]=628.2391, found 628.2393.

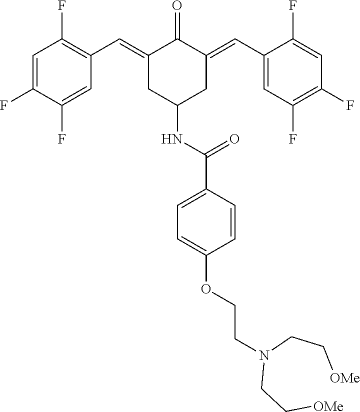 [SMILES: COCCN(CCOC)CCOc1ccc(C(=O)NC2C/C(=C\c3cc(F)c(F)cc3F)C(=O)/C(=C/c3cc(F)c(F)cc3F)C2)cc1]

### 4-(2-(Bis(2-methoxyethyl)amino)ethoxy)-N-(4-oxo-3,5-bis((E)-2,4,5-trifluorobenzylidene)cyclo-hexyl)benzamide (JCS155)

¹H NMR (500 MHz, CDCl₃) δ 7.81 (s, 2H), 7.65 (d, J=8.8 Hz, 2H), 7.22-7.13 (m, 2H), 7.02-6.94 (m, 2H), 6.89 (d, J=8.8 Hz, 2H), 6.32 (d, J=7.3 Hz, 1H)), 4.52-4.42 (m, 1H), 4.11 (t, J=6.0 Hz, 2H), 3.52 (t, J=5.8 Hz, 4H), 3.36 (s, 6H), 3.20-2.92 (m, 6H), 2.88 (t, J=5.8 Hz, 4H).

¹³C NMR (126 MHz, CDCl₃) δ 187.1, 166.6, 161.7, 156.3 (dd, J_{C-F}=252.0, 8.8 Hz, 2C), 150.6 (dt, J_{C-F}=254.5, 12.6 Hz, 2C), 146.7 (dd, J_{C-F}=243.1, 11.3 Hz, 2C), 135.0 (2C), 130.9 (2C), 128.7 (2C), 126.1, 119.6 (dd, J_{C-F}=15.1, 5.0 Hz, 2C), 118.1 (dd, J_{C-F}=20.1, 3.7 Hz, 2C), 114.3 (2C), 106.2 (dd, J_{C-F}=27.7, 3.7 Hz, 2C), 71.1, 66.7, 58.9, 54.7, 53.8, 44.6, 34.0.

¹⁹F NMR (376 MHz, CDCl₃) δ −112.4 (d, J_{FF}=18.8, 11.2 Hz, 2F), −130.3 (d, J_{FF}=30.0, 7.5 Hz, 2F), −141.5 (d, J_{FF}=30.0, 18.8 Hz, 2F).

HR-APCI m/z calcd for C₃₅H₃₅F₆N₂O₅ [M+H]=677.2442, found 677.2442.

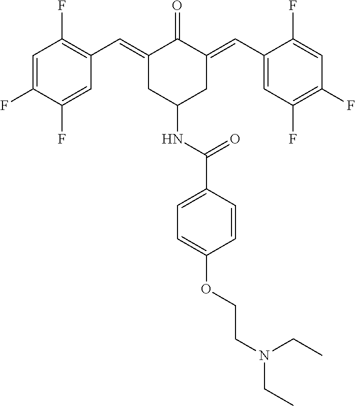 [SMILES: CCN(CC)CCOc1ccc(C(=O)NC2C/C(=C\c3cc(F)c(F)cc3F)C(=O)/C(=C/c3cc(F)c(F)cc3F)C2)cc1]

### 4-(2-(Diethylamino)ethoxy)-N-(4-oxo-3,5-bis((E)-2,4,5-trifluorobenzylidene)cyclohexyl)benzamide (JCS156)

¹H NMR (500 MHz, CDCl₃) δ 7.79 (s, 2H), 7.64 (d, J=8.8 Hz, 2H), 7.22-7.13 (m, 2H), 7.02-6.94 (m, 2H), 6.86 (d, J=8.8 Hz, 2H), 6.35 (d, J=7.4 Hz, 1H), 4.52-4.42 (m, 1H), 4.15 (t, J=5.9 Hz, 2H), 3.12 (br d, J=15.6 Hz, 2H), 3.05-2.90 (m, 4H), 2.75 (q, J=7.2 Hz, 4H), 1.13 (t, J=7.2 Hz, 6H).

¹³C NMR (126 MHz, CDCl₃) δ 187.1, 166.6, 161.4, 156.3 (dd, J_{C-F}=252.0, 8.8 Hz, 2C), 150.6 (dt, J_{C-F}=254.5, 12.6 Hz, 2C), 146.7 (dd, J_{C-F}=243.1, 11.3 Hz, 2C), 135.1 (2C), 130.9 (2C), 128.9 (2C), 126.4, 119.5 (dd, J_{C-F}=15.1, 5.0 Hz, 2C), 118.2 (dd, J_{C-F}=20.1, 3.7 Hz, 2C), 114.4 (2C), 106.2 (dd, J_{C-F}=27.7, 3.7 Hz, 2C), 66.1, 51.5, 47.8, 44.6, 34.0, 11.2.

¹⁹F NMR (376 MHz, CDCl₃) δ −112.4 (d, J_{FF}=18.8, 11.2 Hz, 2F), −130.3 (d, J_{FF}=30.0, 7.5 Hz, 2F), −141.5 (d, J_{FF}=30.0, 18.8 Hz, 2F).

HR-APCI m/z calcd for C₃₃H₃₁F₆N₂O₃ [M+H]=617.2231, found 617.2233.

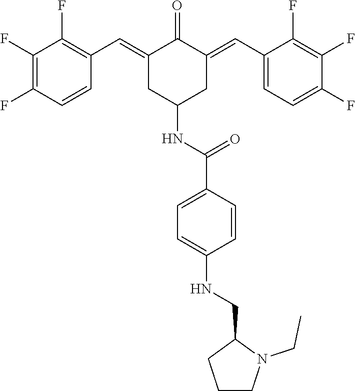 [SMILES: CCN1CCC[C@H]1CNc1ccc(C(=O)NC2C/C(=C\c3ccc(F)c(F)c3F)C(=O)/C(=C/c3ccc(F)c(F)c3F)C2)cc1]

### 4-((((S)-1-Ethylpyrrolidin-2-yl)methyl)amino)-N-(4-oxo-3,5-bis((E)-2,3,4-trifluorobenzylidene)-cyclohexyl)benzamide (JCS157)

¹H NMR (500 MHz, CDCl₃) δ 7.83 (s, 2H), 7.50 (d, J=8.7 Hz, 2H), 7.10-6.95 (m, 4H), 6.53 (d, J=8.7 Hz, 2H), 5.99 (d, J=7.4 Hz, 1H), 4.81 (br s, 1H), 4.52-4.42 (m, 1H), 3.26-3.13 (m, 3H), 3.09 (br d, J=14.3 Hz, 2H), 2.95 (dd, J=15.6, 8.1 Hz, 2H), 2.83-2.77 (m, 1H), 2.26-2.18 (m, 1H), 2.26-2.15 (m, 2H), 1.95-1.65 (m, 4H), 1.09 (t, J=7.2 Hz, 3H).

¹³C NMR (126 MHz, CDCl₃) δ 187.4, 166.9, 151.9, 151.6 (dd, J_{C-F}=254.5, 3.7 Hz, 2C), 149.9 (dd, J_{C-F}=254.5, 3.7 Hz, 2C), 140.6 (dt, J_{C-F}=253.2, 15.5 Hz, 2C), 135.5 (2C), 131.1 (2C), 128.6 (2C), 124.49-124.37 (m, 2C), 121.3, 121.0 (dd, J_{C-F}=11.3, 3.7 Hz, 2C), 112.4 (dd, J_{C-F}=17.6, 3.7 Hz, 2C), 111.8 (2C), 62.6, 53.6, 48.2, 44.4, 44.3, 34.2, 28.6, 22.8, 13.9.

¹⁹F NMR (376 MHz, CDCl₃) δ −131.6 (dd, J_{FF}=26.3, 11.2 Hz, 2F), −131.8 (dd, J_{FF}=30.0, 15.0 Hz, 2F), −159.3 (t, J_{FF}=26.3 Hz, 2F).

HR-APCI m/z calcd for C₃₄H₃₂F₆N₃O₂ [M+H]=628.2395, found 628.2393.

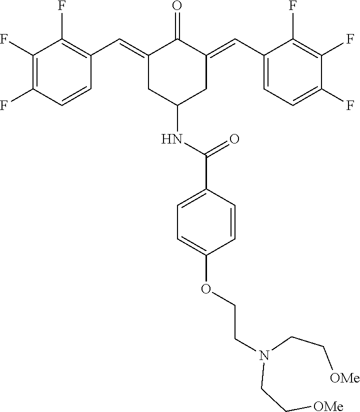 [SMILES: COCCN(CCOC)CCOc1ccc(C(=O)NC2C/C(=C\c3ccc(F)c(F)c3F)C(=O)/C(=C/c3ccc(F)c(F)c3F)C2)cc1]

### 4-(2-(Bis(2-methoxyethyl)amino)ethoxy)-N-(4-oxo-3,5-bis((E)-2,3,4-trifluorobenzylidene)cyclo-hexyl)benzamide (JCS158)

¹H NMR (500 MHz, CDCl₃) δ 7.79 (s, 2H), 7.59 (d, J=8.9 Hz, 2H), 7.07-6.96 (m, 4H), 6.84 (d, J=8.9 Hz, 2H), 6.23 (d, J=7.4 Hz, 1H), 4.47-4.40 (m, 1H), 4.06 (t, J=6.0 Hz, 2H), 3.47 (t, J=5.8 Hz, 4H), 3.31 (s, 6H), 3.09 (br d, J=13.8 Hz, 2H), 3.01 (t, J=6.0 Hz, 2H), 2.94 (dd, J=15.5, 8.4 Hz, 2H), 2.83 (t, J=5.8 Hz, 4H).

¹³C NMR (126 MHz, CDCl₃) δ 187.1, 166.5, 161.6, 151.6 (dd, J_{C-F}=254.5, 3.7 Hz, 2C), 149.9 (dd, J_{C-F}=254.5, 3.7 Hz, 2C), 140.6 (dt, J_{C-F}=253.2, 15.5 Hz, 2C), 135.2 (2C), 131.0 (2C), 128.6 (2C), 126.0, 124.37-124.24 (m, 2C), 120.8 (dd, J_{C-F}=11.3, 3.7 Hz, 2C), 114.3 (2C), 112.3 (dd, J_{C-F}=17.6, 3.7 Hz, 2C), 71.0, 66.7, 58.8, 54.7, 53.7, 44.5, 33.9.

¹⁹F NMR (376 MHz, CDCl₃) δ −131.6 (dd, J_{FF}=26.3, 11.2 Hz, 2F), −131.8 (dd, J_{FF}=30.0, 15.0 Hz, 2F), −159.3 (t, J_{FF}=26.3 Hz, 2F).

HR-APCI m/z calcd for C₃₅H₃₅F₆N₂O₅ [M+H]=677.2446, found 677.2444.

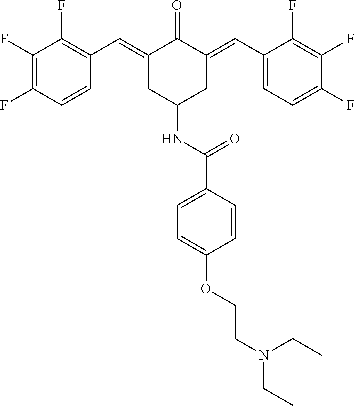 [SMILES: CCN(CC)CCOc1ccc(C(=O)NC2C/C(=C\c3ccc(F)c(F)c3F)C(=O)/C(=C/c3ccc(F)c(F)c3F)C2)cc1]

### 4-(2-(Diethylamino)ethoxy)-N-(4-oxo-3,5-bis((E)-2,3,4-trifluorobenzylidene)cyclohexyl)benzamide (JCS159)

¹H NMR (500 MHz, CDCl₃) δ 7.82 (s, 2H), 7.60 (d, J=8.8 Hz, 2H), 7.10-6.97 (m, 4H), 6.86 (d, J=8.8 Hz, 2H), 6.15 (d, J=7.3 Hz, 1H), 4.48-4.42 (m, 1H), 4.09 (t, J=6.0 Hz, 2H), 3.10 (br d, J=15.7 Hz, 2H), 3.03-2.89 (m, 4H), 2.69 (q, J=7.1 Hz, 4H), 1.08 (t, J=7.2 Hz, 6H).

¹³C NMR (126 MHz, CDCl₃) δ 187.2, 166.6, 161.6, 151.6 (dd, J_{C-F}=254.5, 3.7 Hz, 2C), 149.9 (dd, J_{C-F}=254.5, 3.7 Hz, 2C), 140.6 (dt, J_{C-F}=253.2, 15.5 Hz, 2C), 135.3 (2C), 131.2 (2C), 128.7 (2C), 126.2, 124.46-124.36 (m, 2C), 120.9 (dd, J_{C-F}=11.3, 3.7 Hz, 2C), 114.4 (2C), 112.4 (dd, J_{C-F}=17.6, 3.7 Hz, 2C), 66.5, 51.5, 47.8, 44.6, 34.1, 11.4.

¹⁹F NMR (376 MHz, CDCl₃) δ −131.4 (dd, J_{FF}=26.3, 11.2 Hz, 2F), −131.8 (dd, J_{FF}=30.0, 15.0 Hz, 2F), −159.3 (t, J_{FF}=26.3 Hz, 2F).

HR-APCI m/z calcd for C₃₃H₃₁F₆N₂O₃ [M+H]=617.2237, found 617.2233.

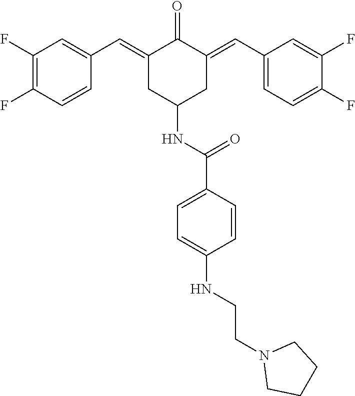 [SMILES: O=C1/C(=C/c2ccc(F)c(F)c2)CC(NC(=O)c2ccc(NCCN3CCCC3)cc2)C/C1=C\c1ccc(F)c(F)c1]

### N-(3,5-Bis((E)-3,4-difluorobenzylidene)-4-oxocyclohexyl)-4-((2-(pyrrolidin-1-yl)ethyl)amino)benz-amide (JCSO27)

To the mixture of the N-tert-butyl (4-oxocyclohexyl)carbamate (213.28 mg, 1 mmol, 1.0 equiv.) and ethanol (1.0 mL) in a round bottom flask added dropwise 20% aq. sodium hydroxide (1.5 mL) and stirred for five minutes. To this mixture was added 3,4-difluorobenzaldehyde (355.3 mg, 2.5 mmol, 2.5 equiv.). The reaction mixture was then allowed to stir at 21° C. for 5 h. After 5 h the yellow precipitate thus obtained was filtered, washed with water, cold ethanol and dried to get the pure product (360 mg, 78% yield).

Trifluoroacetic acid (0.5 ml) was added to a solution of N-tert-butyl (3,5-bis((E)-3,4-difluorobenzylidene)-4-oxocyclohexyl)carbamate (230.7 mg, 0.5 mmol) in dichloromethane (5.0 ml) at 21° C. and stirred overnight at 21° C. Then, the solvent of the reaction solution was distilled off under reduced pressure and the resulting residue was poured into a 1N aqueous sodium hydroxide solution and extracted with ethyl acetate. The organic layer was washed with a saturated aqueous sodium chloride solution and then dried over anhydrous magnesium sulfate. The solvent was distilled off under reduced pressure to obtain 4-amino-2,6-bis((E)-3,4-difluorobenzylidene)cyclohexan-1-one.

The mixture of 4-amino-2,6-bis((E)-3,4-difluorobenzylidene)cyclohexan-1-one (180.7 mg, 0.5 mmol, 1.0 equiv.) and anhydrous diisopropylethylamine (261.2 μL, 1.5 mmol, 3.0 equiv.) in THE was maintained at 0° C. (ice bath). To this cooled mixture was added dropwise 4-((2-(pyrrolidin-1-yl)ethyl)amino)benzoic acid (117.0 mg, 0.5 mmol, 1.0 equiv.) in 2.0 mL THF followed by and TBTU (240.8 mg, 0.75 mmol, 1.5 equiv.). After the complete addition of 4-((2-(pyrrolidin-1-yl)ethyl)amino)-benzoic acid, the reaction mixture was slowly warmed up to room temperature and stirred overnight. After completion of the reaction, the solvent was evaporated and the residue was stirred in sat. aq, NaHCO₃ for 5 min. The mixture was extracted with ethyl acetate three times. The organic layer was washed with a saturated aqueous sodium chloride solution and then dried over anhydrous sodium sulfate. The solvent was evaporated, followed by flash chromatography (gradient elution 50% methanol/ethyl acetate-100% methanol) to give desired product JCSO27 (0.172 mg, 60% yield) as yellow solid.

### Synthesis of 4-((2-(pyrrolidin-1-yl)ethyl)amino)benzoic acid

To a microwave vial equipped with a stir bar was added tert-butyl 4-fluorobenzoate (0.502 g, 2.55 mmol, 1.0 equiv) and 2-(pyrrolidin-1-yl)ethan-1-amine (1.16 g, 10.2 mmol, 4.0 equiv). The mixture was heated in microwave at 120° C. for 24 h, after which time diluted with ethyl acetate (15 mL), washed with saturated aqueous sodium bicarbonate (10 mL)), brine (10 nmL), dried over sodium sulfate and evaporated to give the crude. The pure product was obtained by flash chromatography on silica gel (gradient elution 10% methanol/EtOAc-100% methanol) to give the desired product, tert-butyl 4-((2-(pyrrolidin-1-yl)ethyl)amino)benzoate (0.44 g, 60% yield), as a light yellow oil. Tert-butyl 4-((2-(pyrrolidin-1-yl)ethyl)amino)benzoate (0.29 g, 1.0 mmol, 1.0 equiv) was dissolved in 6 mL dichloromethane and trifluoroacetic acid (2 mL) was added dropwise. The mixture was stirred at 21° C. for 5 h. The dichloromethane was removed in vacuo and the crude residue was washed with dichloromethane and evaporated several times to get rid of excess trifluoroacetic acid to provide 4-((2-(pyrrolidin-1-yl)ethyl)amino)benzoic acid (0.22 g, 90% yield) as brown solid which was pure enough to be used for next step.

### N-(3,5-Bis((E)-3,4-difluorobenzylidene)-4-oxocyclohexyl)-4-((2-(pyrrolidin-1-yl)ethyl)amino)benz-amide (JCSO27)

¹H NMR (500 MHz, CDCl₃) δ 7.78 (s, 2H), 7.53 (d, J=8.7 Hz, 2H), 7.27-7.15 (m, 6H), 6.54 (d, J=8.7 Hz, 2H), 6.07 (d, J=7.3 Hz, 1H), 4.85 (t, J=5.2 Hz, 1H), 4.53-4.45 (m, 1H), 3.30-3.18 (m, 4H), 3.04 (dd, J=17.0, 8.1 Hz, 2H), 2.77 (t, J=6.0 Hz, 2H), 2.59 (t, J=6.3 Hz, 4H), 1.83-1.80 (m, 4H). ¹³C NMR (126 MHz, CDCl₃) δ 188.1, 167.0, 151.3, 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 137.7 (2C), 133.0 (2C), 132.3 (dd, J_{C-C-CF}=6.3 Hz, J_{C-C-CF}=3.7 Hz, 2C), 128.7 (2C), 127.1 (dd, J_{C-C-CF}=6.3 Hz, J_{C-C-CF}=3.7 Hz, 2C), 121.7, 119.0 (d, J_{C-CF}=17.6 Hz, 2C), 117.7 (d, J_{C-CF}=17.6 Hz, 2C), 111.8 (2C), 54.5, 54.0, 44.4, 41.6, 33.9, 23.5. ¹⁹F NMR (376 MHz, CDCl₃) δ −135.2 (d, J_{FF}=18.8 Hz, 2F),−136.6 (d, J_{FF}=22.5 Hz, 2F). HR-APCI m/z calcd for C₃₃H₃₂F₄N₃O₂ [M+H]=578.2425, found 578.2416.

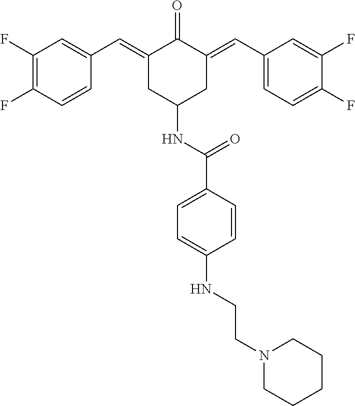 [SMILES: O=C1/C(=C/c2ccc(F)c(F)c2)CC(NC(=O)c2ccc(NCCN3CCCCC3)cc2)C/C1=C\c1ccc(F)c(F)c1]

### N-(3,5-Bis((E)-3,4-difluorobenzylidene)-4-oxocyclohexyl)-4-((2-(piperidin-1-yl)ethyl)amino)-benzamide (JCSO28)

¹H NMR (500 MHz, CDCl₃) δ 7.76 (s, 2H), 7.53 (d, J=8.6 Hz, 2H), 7.26-7.12 (m, 6H), 6.52 (d, J=8.7 Hz, 2H), 6.15 (d, J=7.3 Hz, 1H), 4.82 (t, J=4.9 Hz, 1H), 4.52-4.44 (m, 1H), 3.22 (bd, J=15.3 Hz, 2H), 3.03 (dd, J=15.5, 8.0 Hz, 2H), 2.56 (t, J=6.0 Hz, 2H), 2.39 (t, J=6.3 Hz, 4H), 1.60-1.54 (m, 4H), 1.47-1.42 (m, 2H). ¹³C NMR (126 MHz, CDCl₃) δ 188.13, 167.03, 151.2, 150.6 (dd, J_{CF}=211.6 Hz, J_{C-CF}=7.5 Hz, 2C), 150.3 (dd, J_{CF}=207.9 Hz, J_{C-CF}=10.0 Hz, 2C), 137.7 (2C), 133.1 (2C), 132.3 (dd, J_{C-C-CF}=5.0 Hz, J_{C-C-CF}=2.5 Hz, 2C), 128.7 (2C), 127.1 (dd, J_{C-C-CF}=6.3 Hz, J_{C-C-CF}=3.7 Hz, 2C), 121.5, 119.0 (d, J_{C-CF}=15.1 Hz, 2C), 117.6 (d, J_{C-CF}=15.1 Hz, 2C), 111.8 (2C), 57.0, 54.3, 44.4, 39.8, 33.9, 26.0, 24.4. ¹⁹F NMR (376 MHz, CDCl₃) δ −135.2 (d, J_{FF}=22.5 Hz, 2F), −136.6 (d, J_{FF}=18.8 Hz, 2F). HR-APCI m/z calcd for C₃₄H₃₄F₄N₃O₂ [M+H]=592.2581, found 592.2578.

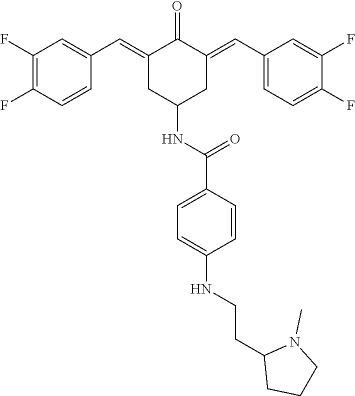 [SMILES: CN1CCCC1CCNc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-((2-(1-methylpyrrolidin-2-yl)ethyl)-amino)benzamide (JCSO41)

¹H NMR (500 MHz, CDCl₃) δ 7.78 (s, 2H), 7.52 (d, J=8.3 Hz, 2H), 7.27-7.14 (m, 6H), 6.49 (d, J=8.3 Hz, 2H), 6.04 (d, J=7.4 Hz, 1H), 4.86 (bs, 1H), 4.52-4.47 (m, 1H), 3.32-3.12 (m, 4H), 3.12-2.99 (m, 3H), 2.32 (s, 3H), 2.25-2.21 (m, 1H), 2.18-2.12 (m, 1H), 1.99-1.82 (m, 2H), 1.77-1.63 (m, 3H), 1.60-1.53 (m, 1H). ¹³C NMR (126 MHz, CDCl₃) δ 188.16, 167.02, 151.5, 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 137.8 (2C), 133.0 (2C), 132.3 (dd, J_{C-C-CF}=6.3 Hz, J_{C-C-CF}=3.7 Hz, 2C), 128.7 (2C), 127.1 (dd, J_{C-C-CF}=6.3 Hz, J_{C-C-CF}=3.7 Hz, 2C), 121.5, 119.0 (d, J_{C-CF}=15.1 Hz, 2C), 117.7 (d, J_{C-CF}=17.6 Hz, 2C), 111.6 (2C), 64.6, 57.2, 44.4, 40.9, 40.8, 33.9, 31.6, 29.9, 22.4. ¹⁹F NMR (376 MHz, CDCl₃) δ 135.2 (d, J_{FF}=22.5 Hz, 2F), 129.6 (d, J_{FF}=30.0 Hz, 2F). HR-APCI m/z calcd for C₃₄H₃₄F₄N₃O₂ [M+H]=592.2581, found 592.2578.

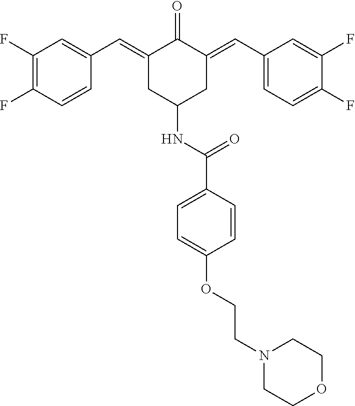 [SMILES: O=C1/C(=C/c2ccc(F)c(F)c2)CC(NC(=O)c2ccc(OCCN3CCOCC3)cc2)C/C1=C\c1ccc(F)c(F)c1]

### N-(3,5-Bis((E)-3,4-difluorobenzylidene)-4-oxocyclohexyl)-4-((2-morpholinoethyl)amino)benzamide (JCSO42)

¹H NMR (500 MHz, CDCl₃) δ 7.79 (s, 2H), 7.53 (d, J=8.7 Hz, 2H), 7.27-7.14 (m, 6H), 6.54 (d, J=8.7 Hz, 2H), 6.02 (d, J=7.3 Hz, 1H), 4.72 (bs, 1H), 4.53-4.47 (m, 1H), 3.72 (t, J=4.7 Hz, 4H), 3.24 (bd, J=15.0 Hz, 2H), 3.18 (t, J=6.0 Hz, 2H), 3.05 (dd, J=15.3, 8.1 Hz, 2H), 2.63 (t, J=5.9 Hz, 2H), 2.47 (t, J=4.7 Hz, 4H). ¹³C NMR (126 MHz, CDCl₃) δ 188.17, 166.98, 151.6, 150.7 (dd, J_{CF}=211.6 Hz, J_{C-CF}=11.3 Hz, 2C), 150.3 (dd, J_{CF}=209.1 Hz, J_{C-CF}=11.3 Hz, 2C), 137.8 (2C), 133.0 (2C), 132.3 (dd, J_{C-C-CF}=6.3 Hz, J_{C-C-CF}=3.7 Hz, 2C), 128.7 (2C), 127.1 (dd, J_{C-C-CF}=6.3 Hz, J_{C-C-CF}=3.7 Hz, 2C), 121.9, 119.0 (d, J_{C-CF}=13.8 Hz, 2C), 117.7 (d, J_{C-CF}=15.1 Hz, 2C), 111.9 (2C), 67.0, 56.8, 53.4, 44.4, 39.3, 33.9. ¹⁹F NMR (376 MHz, CDCl₃) δ −135.1 (d, J_{FF}=18.8 Hz, 2F), −136.5 (d, J_{FF}=18.8 Hz, 2F). HR-APCI m/z calcd for C₃₄H₃₂F₄N₃O₃ [M+H]=594.2374, found 594.2366.

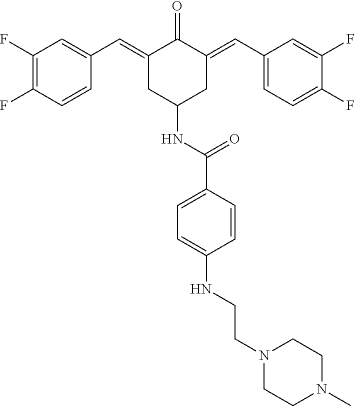 [SMILES: CN1CCN(CCNc2ccc(C(=O)NC3C/C(=C\c4ccc(F)c(F)c4)C(=O)/C(=C/c4ccc(F)c(F)c4)C3)cc2)CC1]

### N-(3,5-Bis((E)-3,4-difluorobenzylidene)-4-oxocyclohexyl)-4-((2-(4-methylpiperazin-1-yl)ethyl)amino)benzamide (JCSO43)

¹H NMR (500 MHz, CDCl₃) δ 7.81 (s, 2H), 7.52 (d, J=8.7 Hz, 2H), 7.29-7.18 (m, 1H), 6.55 (d, J=8.7 Hz, 2H), 5.95 (d, J=7.3 Hz, 1H), 4.72 (t, J=5.0 Hz, 1H), 4.54-4.48 (m, 1H), 3.25 (bd, J=15.0 Hz, 2H), 3.17 (q, J=5.3 Hz, 2H), 3.06 (dd, J=15.3, 8.0 Hz, 2H), 2.63 (t, J=5.9 Hz, 2H), 2.58-2.41 (m, 8H), 2.30 (s, 3H). ¹³C NMR (126 MHz, CDCl₃) δ 188.2, 166.9, 151.4, 150.8 (dd, J_{CF}=254.5 Hz, J_{C-CF}=11.3 Hz, 2C), 150.3 (dd, J_{CF}=249.4 Hz, J_{C-CF}=11.3 Hz, 2C), 137.9 (2C), 133.0 (2C), 132.3 (dd, J_{C-C-CF}=6.3 Hz, J_{C-C-CF}=3.7 Hz, 2C), 128.7 (2C), 127.1 (dd, J_{C-C-CF}=6.3 Hz, J_{C-C-CF}=3.7 Hz, 2C), 121.8, 119.1 (d, J_{C-CF}=17.6 Hz, 2C), 117.8 (d, J_{C-CF}=17.6 Hz, 2C), 111.9 (2C), 56.3, 55.2, 52.8, 46.1, 44.4, 39.7, 34.0. ¹⁹F NMR (376 MHz, CDCl₃) δ 131.0 (d, J_{FF}=30.0 Hz, 2F), 129.6 (d, J_{FF}=30.0 Hz, 2F). HR-APCI m/z calcd for C₃₄H₃₅F₄N₄O₂ [M+H]=607.2690, found 607.2671.

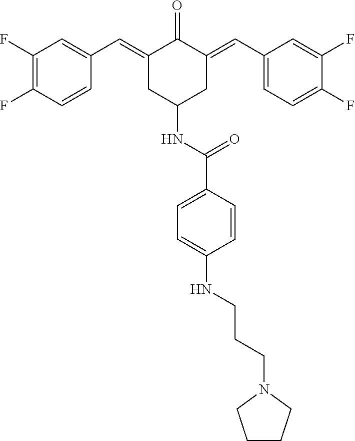 [SMILES: O=C1/C(=C/c2ccc(F)c(F)c2)CC(NC(=O)c2ccc(NCCCN3CCCC3)cc2)C/C1=C\c1ccc(F)c(F)c1]

### N-(3,5-Bis((E)-3,4-difluorobenzylidene)-4-oxocyclohexyl)-4-((3-(pyrrolidin-1-yl)propyl)amino)benz-amide (JCSO44)

¹H NMR (500 MHz, CDCl₃) δ 7.80 (s, 2H), 7.52 (d, J=8.3 Hz, 2H), 7.25-7.15 (m, 6H), 6.55 (d, J=8.3 Hz, 2H), 5.95 (d, J=7.4 Hz, 1H), 4.70 (bs, 1H), 4.54-4.47 (m, 1H), 3.71 (t, J=4.6 Hz, 4H), 3.24 (bd, J=15.0 Hz, 2H), 3.18 (t, J=5.7 Hz, 2H), 3.05 (dd, J=15.6, 7.8 Hz, 2H), 2.63 (t, J=5.7 Hz, 2H), 2.46 (t, J=4.5 Hz, 4H), 1.64-1.54 (m, 1H). ¹³C NMR (126 MHz, CDCl₃) δ 188.1, 166.9, 151.3, 150.8 (dd, J_{CF}=254.5 Hz, J_{C-CF}=11.3 Hz, 2C), 150.3 (dd, J_{CF}=249.4 Hz, J_{C-CF}=11.3 Hz, 2C), 137.9 (2C), 133.0 (2C), 132.3 (dd, J_{C-C-CF}=6.3 Hz, J_{C-C-CF}=3.7 Hz, 2C), 128.7 (2C), 127.2 (dd, J_{C-C-CF}=6.3 Hz, J_{C-C-CF}=3.7 Hz, 2C), 121.9, 119.1 (d, J_{C-CF}=17.6 Hz, 2C), 117.7 (d, J_{C-CF}=17.6 Hz, 2C), 111.9 (2C), 67.0, 56.8, 53.4 (2C), 44.4, 39.3, 33.9. ¹⁹F NMR (376 MHz, CDCl₃) δ 131.0 (d, J_{FF}=30.0 Hz, 2F), 129.6 (d, J_{FF}=30.0 Hz, 2F). HR-APCI m/z calcd for C₃₄H₃₄F₄N₃O₂ [M+H]=592.2581, found 592.2566.

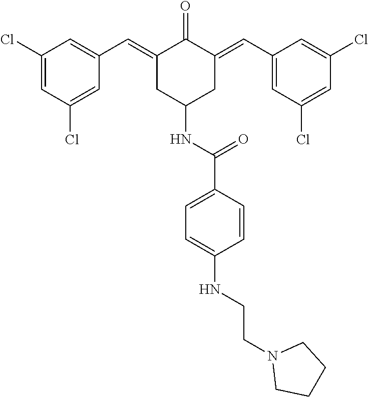 [SMILES: O=C1/C(=C/c2cc(Cl)cc(Cl)c2)CC(NC(=O)c2ccc(NCCN3CCCC3)cc2)C/C1=C\c1cc(Cl)cc(Cl)c1]

### N-(3,5-Bis((E)-3,5-dichlorobenzylidene)-4-oxocyclohexyl)-4-((2-(pyrrolidin-1-yl)ethyl)amino)-benzamide (JCS063)

¹H NMR (500 MHz, CDCl₃) δ 7.73 (s, 2H), 7.56 (d, J=8.3 Hz, 2H), 7.36-7.34 (m, 2H), 7.28-7.26 (m, 4H), 6.56 (d, J=8.4 Hz, 2H), 6.12 (d, J=7.5 Hz, 1H), 4.73 (t, J=5.1 Hz, 1H), 4.56-4.51 (m, 1H), 3.27-3.16 (m, 4H), 3.08 (dd, J=15.2, 7.9 Hz, 2H), 2.74 (t, J=6.0 Hz, 2H), 2.53 (t, J=5.6 Hz, 4H), 1.80 (t, J=6.6 Hz, 4H). ¹³C NMR (126 MHz, CDCl₃) δ 187.8, 167.0, 151.5, 138.0 (2C), 137.3 (2C), 135.3, 134.4 (2C), 129.0 (2C), 128.7 (2C), 128.3, 121.5 (2C), 111.8, 54.5, 53.9, 44.2, 41.9, 33.9, 23.6. HR-APCI m/z calcd for C₃₃H₃₂Cl₄N₃O₂ [M+H]=642.1243, found 642.1222.

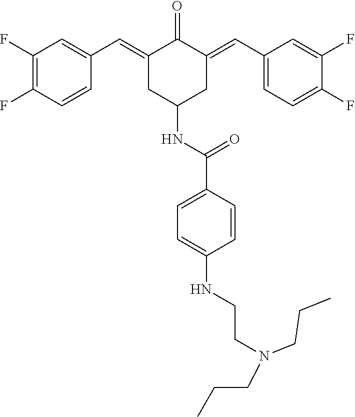 [SMILES: CCCN(CCC)CCNc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-((2-(dipropylamino)ethyl)amino)benz-amide (JCS065)

¹H NMR (500 MHz, CDCl₃) δ 7.80 (s, 2H), 7.51 (d, J=8.3 Hz, 2H), 7.28-7.18 (m, 6H), 6.52 (d, J=8.3 Hz, 2H), 5.98 (d, J=7.3 Hz, 1H), 4.94 (bs, 1H), 4.54-4.45 (m, 1H), 3.30-3.01 (m, 6H), 2.71 (t, J=5.0 Hz, 2H), 2.43 (t, J=7.6 Hz, 4H), 1.45 (q, J=7.5 Hz, 4H), 0.87 (t, J=7.4 Hz, 6H). ¹³C NMR (126 MHz, CDCl₃) δ 188.2, 167.0, 151.6, 150.7 (dd, J_{CF}=253.2 Hz, J_{C-CF}=12.6 Hz, 2C), 150.3 (dd, J_{CF}=250.7 Hz, J_{C-CF}=12.6 Hz, 2C), 137.8 (2C), 133.0 (2C), 132.3 (dd, J_{C-C-CF}=6.3 Hz, J_{C-C-CF}=3.7 Hz, 2C), 128.7 (2C), 127.1 (dd, J_{C-C-CF}=6.3 Hz, J_{C-C-CF}=3.7 Hz, 2C), 121.6, 119.0 (d, J_{C-CF}=17.6 Hz, 2C), 117.7 (d, J_{C-CF}=17.6 Hz, 2C), 112.0 (2C), 55.7, 52.4, 44.4, 40.6, 34.0, 20.1, 12.0. ¹⁹F NMR (376 MHz, CDCl₃) δ −135.2 (d, J_{FF}=18.8 Hz, 2F), −136.6 (d, J_{FF}=22.5 Hz, 2F). HR-APCI m/z calcd for C₃₅H₃₈F₄N₃O₂ [M+H]=608.2894, found 608.2891.

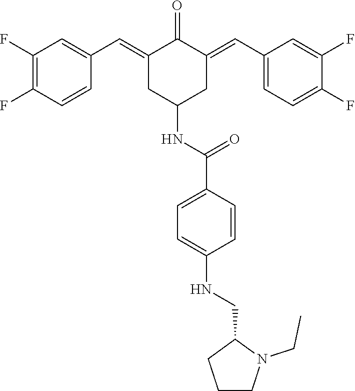 [SMILES: CCN1CCC[C@@H]1CNc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-((((R)-1-ethylpyrrolidin-2-yl)methyl)-amino)benzamide (JCS066)

¹H NMR (500 MHz, CDCl₃) δ 7.78 (s, 2H), 7.52 (d, J=8.7 Hz, 2H), 7.26-7.16 (m, 6H), 6.53 (d, J=8.7 Hz, 2H), 6.04 (d, J=7.3 Hz, 1H), 4.75 (bs, 1H), 4.52-4.46 (m, 1H), 3.27-3.10 (m, 5H), 3.04 (dd, J=15.7, 9.0 Hz, 2H), 2.82-2.75 (m, 1H), 2.67-2.63 (m, 1H), 2.24-2.15 (m, 2H), 1.93-1.85 (m, 1H), 1.77-1.66 (m, 3H), 1.08 (t, J=7.2 Hz, 3H). ¹³C NMR (126 MHz, CDCl₃) δ 188.1, 167.0, 151.7, 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 137.8 (2C), 133.1 (2C), 132.3 (dd, J_{C-C-CF}=6.3 Hz, J_{C-C-CF}=3.7 Hz, 2C), 128.7 (2C), 127.1 (dd, J_{C-C-CF}=6.3 Hz, J_{C-C-CF}=3.7 Hz, 2C), 121.4, 119.0 (d, J_{C-CF}=17.6 Hz, 2C), 117.7 (d, J_{C-CF}=17.6 Hz, 2C), 111.8 (2C), 62.9, 53.7, 48.5, 44.4 (2C), 33.9, 28.6, 22.8, 13.6. ¹⁹F NMR (376 MHz, CDCl₃) δ 130.9 (d, J_{FF}=30.0 Hz, 2F), 129.6 (d, J_{FF}=22.5 Hz, 2F). HR-APCI m/z calcd for C₃₄H₃₅F₄N₃O₂ [M+H]=592.2581, found 592.2576.

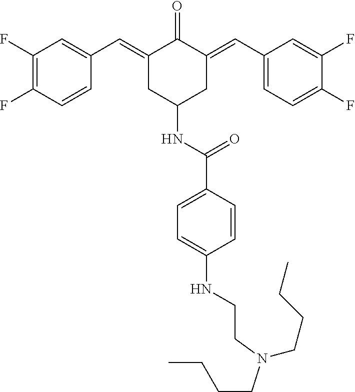 [SMILES: CCCCN(CCCC)CCNc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-((2-(dibutylamino)ethyl)amino)-benzamide (JCS067)

¹H NMR (500 MHz, CDCl₃) δ 7.74 (s, 2H), 7.54 (d, J=8.7 Hz, 2H), 7.27-7.13 (m, 6H), 6.53 (d, J=8.6 Hz, 2H), 6.49 (d, J=7.4 Hz, 1H), 4.44-4.37 (m, 1H), 3.50 (t, J=5.7 Hz, 2H), 3.29-3.16 (m, 4H), 3.12-2.91 (m, 6H), 1.67-1.55 (m, 4H), 1.35-1.28 (m, 4H), 0.89 (t, J=7.3 Hz, 6H). ¹³C NMR (126 MHz, CDCl₃) δ 188.0, 167.0, 150.6 (dd, J_{CF}=253.2 Hz, J_{C-CF}=12.6 Hz, 2C), 150.2 (dd, J_{CF}=249.4 Hz, J_{C-CF}=12.6 Hz, 2C), 150.0, 137.4 (2C), 133.2 (2C), 132.3 (dd, J_{C-C-CF}=6.3 Hz, J_{C-C-CF}=3.7 Hz, 2C), 129.0 (2C), 127.2 (dd, J_{C-C-CF}=6.3 Hz, J_{C-C-CF}=3.7 Hz, 2C), 122.7, 119.0 (d, J_{C-CF}=17.6 Hz, 2C), 117.7 (d, J_{C-CF}=17.6 Hz, 2C), 111.9 (2C), 53.3, 52.1, 44.8, 38.7, 34.0, 25.5, 20.0, 13.6. ¹⁹F NMR (376 MHz, CDCl₃) δ 130.8 (d, J_{FF}=30.0 Hz, 2F), 129.5 (d, J_{FF}=30.0 Hz, 2F). HR-APCI m/z calcd for C₃₇H₄₂F₄N₃O₂ [M+H]=636.3207, found 636.3208.

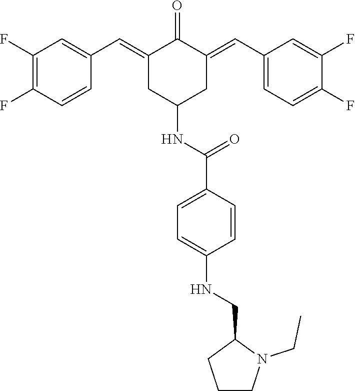 [SMILES: CCN1CCC[C@H]1CNc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-((((S)-1-ethylpyrrolidin-2-yl)methyl)-amino)benzamide (JCS069)

¹H NMR (500 MHz, CDCl₃) δ 7.78 (s, 2H), 7.51 (d, J=8.5 Hz, 2H), 7.25-7.15 (m, 6H), 6.52 (d, J=8.5 Hz, 2H), 6.04 (d, J=7.3 Hz, 1H), 4.69 (bs, 1H), 4.52-4.46 (m, 1H), 3.26-3.09 (m, 5H), 3.03 (dd, J=15.2, 8.2 Hz, 2H), 2.81-2.74 (m, 1H), 2.65-2.60 (m, 2H), 2.24-2.12 (m, 2H), 1.92-1.86 (m, 1H), 1.76-1.65 (m, 3H), 1.08 (t, J=7.2 Hz, 3H). ¹³C NMR (126 MHz, CDCl₃) δ 188.1, 167.0, 151.9, 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 137.8 (2C), 133.0 (2C), 132.3 (dd, J_{C-C-CF}=6.3 Hz, J_{C-C-CF}=3.7 Hz, 2C), 128.7 (2C), 127.1 (dd, J_{C-C-CF}=6.3 Hz, J_{C-C-CF}=3.7 Hz, 2C), 121.3, 119.0 (d, J_{C-CF}=17.6 Hz, 2C), 117.7 (d, J_{C-CF}=17.6 Hz, 2C), 62.5, 53.6, 48.1, 44.5, 44.3, 33.9, 28.7, 22.7, 14.0. ¹⁹F NMR (376 MHz, CDCl₃) δ 131.1 (d, J_{FF}=26.3 Hz, 2F), 129.6 (d, J_{FF}=26.3 Hz, 2F). HR-APCI m/z calcd for C₃₄H₃₅F₄N₃O₂ [M+H]=592.2581, found 592.2585.

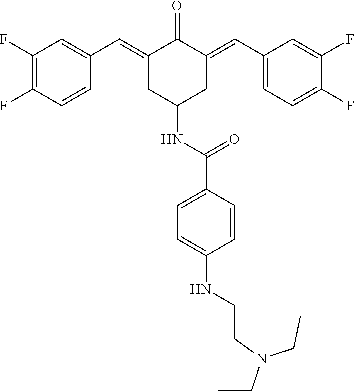 [SMILES: CCN(CC)CCNc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-((2-(diethylamino)ethyl)amino)benz-amide (JCS070)

¹H NMR (500 MHz, CDCl₃) δ 7.78 (s, 2H), 7.52 (d, J=8.7 Hz, 2H), 7.26-7.16 (m, 6H), 6.53 (d, J=8.3 Hz, 2H), 6.03 (d, J=7.3 Hz, 1H), 4.80 (bs, 1H), 4.52-4.46 (m, 1H), 3.23 (bd, J=15.1 Hz, 2H), 3.12 (t, J=6.0 Hz, 2H), 3.04 (dd, J=16.5, 8.4 Hz, 2H), 2.67 (t, J=6.0 Hz, 2H), 2.54 (q, J=7.2 Hz, 4H), 1.01 (t, J=5.0 Hz, 6H). ¹³C NMR (126 MHz, CDCl₃) δ 188.18, 167.03, 151.6, 150.7 (dd, J_{CF}=253.2 Hz, J_{C-CF}=12.6 Hz, 2C), 150.2 (dd, J_{CF}=249.4 Hz, J_{C-CF}=12.6 Hz, 2C), 137.8 (2C), 133.0 (2C), 132.3 (dd, J_{C-C-CF}=6.3 Hz, J_{C-C-CF}=3.7 Hz, 2C), 128.7 (2C), 127.1 (dd, J_{C-C-CF}=6.3 Hz, J_{C-C-CF}=3.7 Hz, 2C), 121.6, 119.0 (d, J_{C-CF}=17.6 Hz, 2C), 117.7 (d, J_{C-CF}=17.6 Hz, 2C), 111.9 (2C), 51.2, 46.6, 44.4, 40.6, 33.9, 11.7. ¹⁹F NMR (376 MHz, CDCl₃) δ 131.1 (d, J_{FF}=30.0 Hz, 2F), 129.6 (d, J_{FF}=30.0 Hz, 2F). HR-APCI m/z calcd for C₃₃H₃₄F₄N₃O₂ [M+H]=580.2581, found 580.2530.

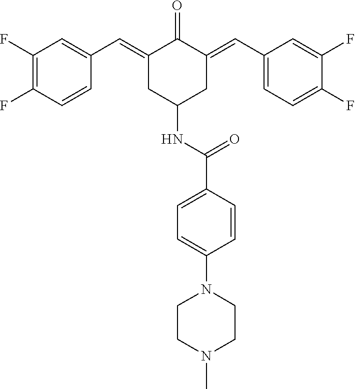 [SMILES: CN1CCN(c2ccc(C(=O)NC3C/C(=C\c4ccc(F)c(F)c4)C(=O)/C(=C/c4ccc(F)c(F)c4)C3)cc2)CC1]

### N-(3,5-Bis((E)-3,4-difluorobenzylidene)-4-oxocyclohexyl)-4-(4-methylpiperazin-1-yl)benzamide (JCS071)

¹H NMR (500 MHz, CDCl₃) δ 7.76 (s, 2H), 7.59 (d, J=8.5 Hz, 2H), 7.26-7.10 (m, 6H), 6.82 (d, J=8.6 Hz, 2H), 6.19 (d, J=7.4 Hz, 1H), 4.52-4.44 (m, 1H), 3.35-3.16 (m, 6H), 3.04 (dd, J=15.4, 8.2 Hz, 2H), 2.52 (t, J=5.1 Hz, 4H), 2.33 (s, 3H). ¹³C NMR (126 MHz, CDCl₃) δ 188.0, 166.7, 153.5, 150.7 (dd, J_{CF}=316.2 Hz, J_{C-CF}=15.1 Hz, 2C), 150.3 (dd, J_{CF}=312.4 Hz, J_{C-CF}=15.1 Hz, 2C), 137.7 (2C), 132.9 (2C), 132.2 (dd, J_{C-C-CF}=6.3 Hz, J_{C-C-CF}=3.7 Hz, 2C), 128.4 (2C), 127.1 (dd, J_{C-C-CF}=6.3 Hz, J_{C-C-CF}=3.7 Hz, 2C), 123.4, 119.0 (d, J_{C-CF}=17.6 Hz, 2C), 117.7 (d, J_{C-CF}=17.6 Hz, 2C), 114.2 (2C), 54.8, 47.8, 46.2, 44.5, 33.8. ¹⁹F NMR (376 MHz, CDCl₃) δ −131.1 (d, J_{FF}=30.0 Hz, 2F), −129.6 (d, J_{FF}=30.0 Hz, 2F). HR-APCI m/z calcd for C₃₂H₃₀F₄N₃O₂ [M+H]=564.2268, found 564.2272.

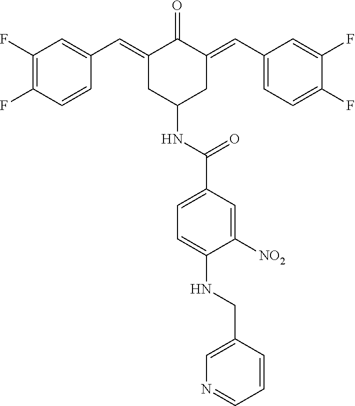 [SMILES: O=C1/C(=C/c2ccc(F)c(F)c2)CC(NC(=O)c2ccc(NCc3cccnc3)c([N+](=O)[O-])c2)C/C1=C\c1ccc(F)c(F)c1]

### N-(3,5-bis((E)-3,4-difluorobenzylidene)-4-oxocyclohexyl)-3-nitro-4-((pyridin-3-ylmethyl)amino)benzamide (JCS082)

¹H NMR (500 MHz, CDCl₃) δ 8.63-8.49 (m, 4H), 7.87 (d, J=8.9 Hz, 1H), 7.70-7.67 (m, 2H), 7.66 (d, J=5.0 Hz, 1H), 7.30-7.09 (m, 7H), 6.76 (d, J=9.1 Hz, 1H), 4.59 (d, J=5.7 Hz, 2H), 4.46-4.39 (m, 1H), 3.27 (bd, J=15.0 Hz, 2H), 3.03 (dd, J=15.0, 9.8 Hz, 2H). ¹³C NMR (126 MHz, CDCl₃) δ 187.8, 165.0, 150.5 (dd, J_{CF}=253.2 Hz, J_{C-CF}=12.6 Hz, 2C), 150.1 (dd, J_{CF}=250.7 Hz, J_{C-CF}=12.6 Hz, 2C), 149.0, 148.3, 146.5, 137.4 (2C), 135.6, 135.3, 133.0 (2C), 132.2 (dd, J_{C-C-CF}=6.3 Hz, J_{C-C-CF}=3.7 Hz, 2C), 131.3, 127.2 (dd, J_{C-C-CF}=6.3 Hz, J_{C-C-CF}=3.7 Hz, 2C), 125.7, 124.1, 121.9, 118.8 (d, J_{C-CF}=17.6 Hz, 2C), 117.6 (d, J_{C-CF}=17.6 Hz, 2C), 114.1 (2C), 45.2, 44.7, 33.7. ¹⁹F NMR (376 MHz, CDCl₃) δ −135.0 (d, J_{FF}=26.6 Hz, 2F), −136.5 (d, J_{FF}=26.6 Hz, 2F). HR-APCI m/z calcd for C₃₃H₂₅F₄N₄O₄ [M+H]=617.1779, found 617.1806.

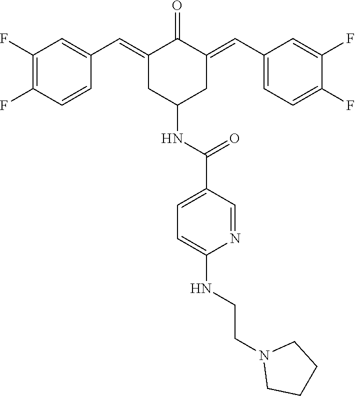 [SMILES: O=C1/C(=C/c2ccc(F)c(F)c2)CC(NC(=O)c2ccc(NCCN3CCCC3)nc2)C/C1=C\c1ccc(F)c(F)c1]

### N-(3,5-bis((E)-3,4-difluorobenzylidene)-4-oxocyclohexyl)-6-((2-(pyrrolidin-1-yl)ethyl)amino)nicotinamide (JCS086)

¹H NMR (500 MHz, CDCl₃) δ 8.40 (s, 1H), 7.76 (s, 2H), 7.73 (d, J=8.9 Hz, 1H), 7.26-7.14 (m, 6H), 6.32 (d, J=8.8 Hz, 1H), 6.20 (d, J=7.2 Hz, 1H), 5.56 (t, J=5.1 Hz, 1H), 4.52-4.44 (m, 1H), 3.37 (t, J=6.0 Hz, 2H), 3.22 (bd, J=15.0 Hz, 2H), 3.06 (dd, J=15.0, 8.0 Hz, 2H), 2.69 (t, J=6.0 Hz, 2H), 2.51 (d, J=6.0 Hz, 4H), 1.85-1.67 (m, 4H). ¹³C NMR (126 MHz, CDCl₃) δ 187.8, 165.8, 160.3, 150.6 (dd, J_{CF}=316.2 Hz, J_{C-CF}=12.6 Hz, 2C), 150.2 (dd, J_{CF}=312.4 Hz, J_{C-CF}=12.6 Hz, 2C), 147.8, 137.8 (2C), 136.3, 132.7 (2C), 132.1 (dd, J_{C-C-CF}=6.3 Hz, J_{C-C-CF}=3.7 Hz, 2C), 127.0 (dd, J_{C-C-CF}=6.3 Hz, J_{C-C-CF}=3.7 Hz, 2C), 118.9 (d, J_{C-CF}=17.6 Hz, 2C), 118.2, 117.6 (d, J_{C-CF}=17.6 Hz, 2C), 106.5, 54.4, 53.8, 44.3, 40.3, 33.7, 23.5. ¹⁹F NMR (376 MHz, CDCl₃) δ −135.0 (d, J_{FF}=26.6 Hz, 2F), −136.5 (d, J_{FF}=26.6 Hz, 2F). HR-APCI m/z calcd for C₃₂H₃₁F₄N₄O₄ [M+H]=579.2389, found 579.2377.

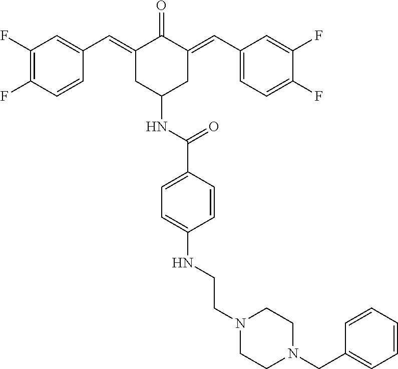 [SMILES: O=C1/C(=C/c2ccc(F)c(F)c2)CC(NC(=O)c2ccc(NCCN3CCN(Cc4ccccc4)CC3)cc2)C/C1=C\c1ccc(F)c(F)c1]

### 4-((2-(4-benzylpiperazin-1-yl)ethyl)amino)-N-(3,5-bis((E)-3,4-difluorobenzylidene)-4-oxocyclohexyl)benzamide (JCS087)

¹H NMR (500 MHz, CDCl₃) δ 7.77 (s, 2H), 7.53 (d, J=8.4 Hz, 2H), 7.34-7.15 (m, 11H), 6.53 (d, J=8.4 Hz, 2H), 6.10 (d, J=7.4 Hz, 1H), 4.76 (s, 1H), 4.51-4.45 (m, 1H), 3.54 (s, 2H), 3.23 (bd, J=15.0 Hz, 2H), 3.17 (t, J=5.9 Hz, 2H), 3.04 (dd, J=15.0, 8.3 Hz, 2H), 2.64 (t, J=5.9 Hz, 2H), 2.52 (m, 8H). ¹³C NMR (126 MHz, CDCl₃) δ 188.0, 166.8, 151.2, 150.6 (dd, J_{CF}=253.2 Hz, J_{C-CF}=12.6 Hz, 2C), 150.1 (dd, J_{CF}=250.7 Hz, J_{C-CF}=12.6 Hz, 2C), 137.6 (2C), 137.4, 132.9 (2C), 132.2 (dd, J_{C-C-CF}=6.3 Hz, J_{C-C-CF}=3.7 Hz, 2C), 129.2 (2C), 128.6, 128.3, 127.2, 127.0 (dd, J_{C-C-CF}=6.3 Hz, J_{C-C-CF}=3.7 Hz, 2C), 121.6, 118.9 (d, J_{C-CF}=17.6 Hz, 2C), 117.6 (d, J_{C-CF}=17.6 Hz, 2C), 111.7 (2C), 62.9, 56.1, 52.7, 52.6, 44.3, 39.5, 33.8. ¹⁹F NMR (376 MHz, CDCl₃) δ 131.0 (d, J_{FF}=26.6 Hz, 2F), 129.6 (d, J_{FF}=26.6 Hz, 2F). HR-APCI m/z calcd for C₄₀H₃₉F₄N₄O₂ [M+H]=683.3018, found 683.3003.

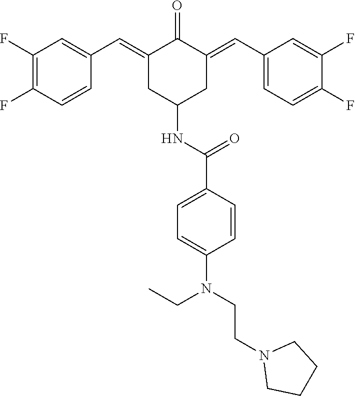 [SMILES: CCN(CCN1CCCC1)c1ccc(C(=O)NC2C/C(=C\c3ccc(F)c(F)c3)C(=O)/C(=C/c3ccc(F)c(F)c3)C2)cc1]

### N-(3,5-bis((E)-3,4-difluorobenzylidene)-4-oxocyclohexyl)-4-(ethyl(2-(pyrrolidin-1-yl)ethyl)amino)benzamide (JCS073)

¹H NMR (500 MHz, CDCl₃) δ 7.75 (s, 2H), 7.59 (d, J=8.5 Hz, 2H), 7.26-7.15 (m, 6H), 6.65 (d, J=8.5 Hz, 2H), 6.29 (d, J=7.1 Hz, 1H), 4.53-4.38 (m, 1H), 3.65 (t, J=5.0 Hz, 2H), 3.40 (q, J=7.2 Hz, 2H), 3.23 (bd, J=15.3 Hz, 2H), 3.05 (dd, J=17.0, 8.1 Hz, 2H), 2.96-2.80 (m, 6H), 2.02-1.88 (m, 4H), 1.13 (t, J=7.1 Hz, 3H). ¹³C NMR (126 MHz, CDCl₃) δ 188.1, 166.8, 150.6 (dd, J_{CF}=254.5 Hz, J_{C-CF}=12.6 Hz, 2C), 150.2 (dd, J_{CF}=249.4 Hz, J_{C-CF}=12.6 Hz, 2C), 149.8, 137.6 (2C), 133.2 (2C), 132.3 (dd, J_{C-C-CF}=6.3 Hz, J_{C-C-CF}=3.7 Hz, 2C), 128.9 (2C), 127.2 (dd, J_{C-C-CF}=6.3 Hz, J_{C-C-CF}=3.7 Hz, 2C), 120.9, 119.0 (d, J_{C-CF}=17.6 Hz, 2C), 117.7 (d, J_{C-CF}=17.6 Hz, 2C), 111.0 (2C), 54.4, 52.6, 48.0, 45.5, 44.5, 34.0, 23.5, 12.2. ¹⁹F NMR (376 MHz, CDCl₃) δ 130.9 (d, J_{FF}=22.5 Hz, 2F), 129.5 (d, J_{FF}=22.5 Hz, 2F). HR-APCI m/z calcd for C₃₅H₃₆F₄N₃O₂ [M+H]=606.2738, found 578.2748.

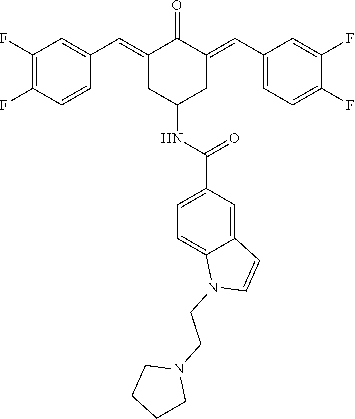 [SMILES: O=C1/C(=C/c2ccc(F)c(F)c2)CC(NC(=O)c2ccc3c(ccn3CCN3CCCC3)c2)C/C1=C\c1ccc(F)c(F)c1]

### N-(3,5-bis((E)-3,4-difluorobenzylidene)-4-oxocyclohexyl)-1-(2-(pyrrolidin-1-yl)ethyl)-1H-indole-5-carboxamide (JCS075)

¹H NMR (500 MHz, CDCl₃) δ 7.97 (d, J=1.5 Hz, 1H), 7.79 (s, 2H), 7.56 (dd, J=8.6, 1.6 Hz, 1H), 7.34 (d, J=8.6 Hz, 1H), 7.27-7.17 (m, 6H), 6.52 (d, J=3.1 Hz, 1H), 6.28 (d, J=7.3 Hz, 1H), 4.57-4.53 (m, 1H), 4.30 (t, J=7.1 Hz, 2H), 3.26 (bd, J=15.0 Hz, 2H), 3.09 (dd, J=15.0, 8.0 Hz, 2H), 2.90 (t, J=7.2 Hz, 2H), 2.56 (t, J=5.0 Hz, 4H), 1.85-1.67 (m, 4H). ¹³C NMR (126 MHz, CDCl₃) δ 188.1, 168.2, 150.7 (dd, J_{CF}=253.2 Hz, J_{C-CF}=12.6 Hz, 2C), 150.3 (dd, J_{CF}=250.7 Hz, J_{C-CF}=12.6 Hz, 2C), 137.9, 137.8, 133.0 (2C), 132.3 (dd, J_{C-C-CF}=6.3 Hz, J_{C-C-CF}=3.7 Hz, 2C), 129.7, 128.7 (2C), 127.1 (dd, J_{C-C-CF}=6.3 Hz, J_{C-C-CF}=3.7 Hz, 2C), 125.4, 120.6, 120.5, 119.1 (d, J_{C-CF}=17.6 Hz, 2C), 117.7 (d, J_{C-CF}=17.6 Hz, 2C), 109.4 (2C), 102.7, 55.7, 54.5, 45.8, 44.6, 33.9, 23.6. ¹⁹F NMR (376 MHz, CDCl₃) δ 131.0 (d, J_{FF}=30.0 Hz, 2F), 129.6 (d, J_{FF}=30.0 Hz, 2F). HR-APCI m/z calcd for C₃₅H₃₂F₄N₃O₂ [M+H]=602.2425, found 602.2418.

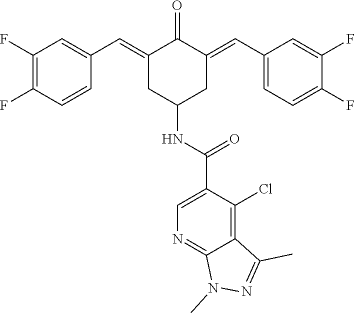 [SMILES: Cc1nn(C)c2ncc(C(=O)NC3C/C(=C\c4ccc(F)c(F)c4)C(=O)/C(=C/c4ccc(F)c(F)c4)C3)c(Cl)c12]

### N-(3,5-bis((E)-3,4-difluorobenzylidene)-4-oxocyclohexyl)-4-chloro-1,3-dimethyl-1H-pyrazolo[3,4-b]pyridine-5-carboxamide (JCS086)

¹H NMR (500 MHz, CDCl₃) δ 8.61 (s, 1H), 7.84 (s, 2H), 7.35-7.11 (m, 6H), 6.43 (d, J=7.1 Hz, 1H), 4.65-4.61 (m, 1H), 4.01 (s, 3H), 3.24 (bd, J=15.6 Hz, 4H), 2.67 (s, 3H). ¹³C NMR (126 MHz, CDCl₃) δ 187.6, 164.4, 151.8, 150.7 (dd, J_{CF}=254.5 Hz, J_{C-CF}=12.6 Hz, 2C), 150.3, 150.2 (dd, J_{CF}=249.4 Hz, J_{C-CF}=12.6 Hz, 2C), 141.8, 138.4 (2C), 135.6 (2C), 132.1, 132.0 (dd, J_{C-C-CF}=6.3 Hz, J_{C-C-CF}=3.7 Hz, 2C), 127.0 (dd, J_{C-C-CF}=6.3 Hz, J_{C-C-CF}=3.7 Hz, 2C), 122.2, 118.9 (d, J_{C-CF}=22.6 Hz, 2C), 117.7 (d, J_{C-CF}=22.6 Hz, 2C), 112.4, 44.9, 33.8, 33.1, 14.7. ¹⁹F NMR (376 MHz, CDCl₃) δ 131.4 (d, J_{FF}=26.3 Hz, 2F), 129.9 (d, J_{FF}=26.3 Hz, 2F). HR-APCI m/z calcd for C₂₉H₂₂C1F₄N₄O₂ [M+H]=569.1439, found 569.2319.

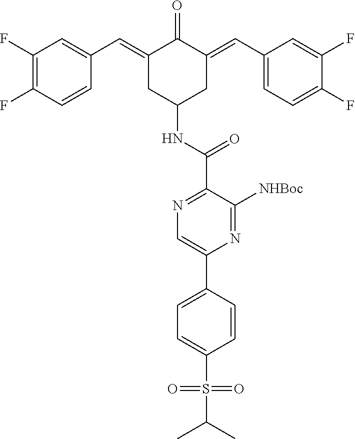 [SMILES: CC(C)S(=O)(=O)c1ccc(-c2cnc(C(=O)NC3C/C(=C\c4ccc(F)c(F)c4)C(=O)/C(=C/c4ccc(F)c(F)c4)C3)c(NC(=O)OC(C)(C)C)n2)cc1]

### tert-butyl (3-((3,5-bis((E)-3,4-difluorobenzylidene)-4-oxocyclohexyl)carbamoyl)-6-(4-(isopropylsulfonyl)phenyl)pyrazin-2-yl)carbamate (JCS088)

¹H NMR (500 MHz, DMSO-d₆) δ 11.05 (s, 1H), 9.25 (s, 1H), 9.16 (d, J=7.9 Hz, 1H), 8.52 (d, J=8.6 Hz, 2H), 8.00 (d, J=8.6 Hz, 2H), 7.71-7.42 (m, 8H), 4.31-4.18 (m, 1H), 3.56-3.48 (m, 1H), 3.28-3.14 (m, 4H), 1.41 (s, 9H), 1.20 (d, J=6.8 Hz, 6H). ¹³C NMR (126 MHz, DMSO-d₆) δ 187.4, 164.8, 149.7, 149.6 (dd, J_{CF}=250.7 Hz, J_{C-CF}=12.6 Hz, 2C), 149.3 (dd, J_{CF}=245.7 Hz, J_{C-CF}=12.6 Hz, 2C), 147.8, 143.5, 141.6, 139.8, 137.2, 135.4 (2C), 134.4 (2C), 132.6 (dd, J_{C-C-CF}=6.3 Hz, J_{C-C-CF}=3.7 Hz, 2C), 129.7, 129.0 (2C), 127.6 (dd, J_{C-C-CF}=6.3 Hz, J_{C-C-CF}=3.7 Hz, 2C), 127.2 (2C), 119.2 (d, J_{C-CF}=17.6 Hz, 2C), 118.4 (2C), 117.8 (d, J_{C-CF}=17.6 Hz, 2C), 80.3, 54.1, 45.0, 32.7, 27.7, 15.1. ¹⁹F NMR (376 MHz, DMSO-d₆) δ −134.6 (d, J_{FF}=18.8 Hz, 2F), −136.2 (d, J_{FF}=22.5 Hz, 2F). HR-APCI m/z calcd for C₃₉H₃₇F₄N₄O₆S [M+H]=765.2397, found 765.2364.

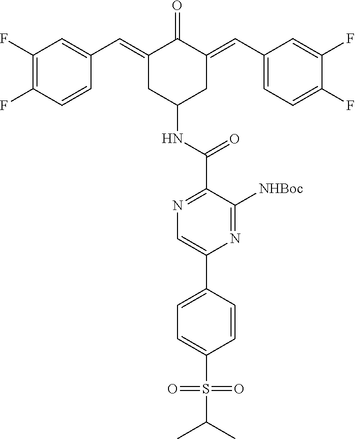 [SMILES: CC(C)S(=O)(=O)c1ccc(-c2cnc(C(=O)NC3C/C(=C\c4ccc(F)c(F)c4)C(=O)/C(=C/c4ccc(F)c(F)c4)C3)c(NC(=O)OC(C)(C)C)n2)cc1]

### 3-amino-N-(3,5-bis((E)-3,4-difluorobenzylidene)-4-oxocyclohexyl)-5-(4-(isopropylsulfonyl)phenyl)pyrazine-2-carboxamide (JCS089)

¹H NMR (500 MHz, DMSO-d₆) δ 8.97 (s, 1H), 8.77 (d, J=7.8 Hz, 1H), 8.39 (d, J=8.2 Hz, 2H), 7.92 (d, J=8.2 Hz, 2H), 7.74-7.44 (m, 8H), 4.29-4.17 (m, 1H), 3.53-3.41 (m, 1H), 3.27-3.14 (m, 4H), 1.19 (d, J=6.9 Hz, 6H). ¹³C NMR (126 MHz, DMSO-d₆) δ 188.0, 165.8, 154.9, 150.1 (dd, J_{CF}=252.0 Hz, J_{C-CF}=12.6 Hz, 2C), 149.7 (dd, J_{CF}=245.7 Hz, J_{C-CF}=12.6 Hz, 2C), 145.6, 141.3, 136.5, 136.1, 135.9 (2C), 134.9 (2C), 133.0 (dd, J_{C-C-CF}=6.3 Hz, J_{C-C-CF}=3.7 Hz, 2C), 129.4 (2C), 128.0 (dd, J_{C-C-CF}=6.3 Hz, J_{C-C-CF}=3.7 Hz, 2C), 126.3 (2C), 124.7, 119.7 (d, J_{C-CF}=17.6 Hz, 2C), 118.2 (d, J_{C-CF}=17.6 Hz, 2C), 54.6, 45.0, 33.3, 15.6. ¹⁹F NMR (376 MHz, DMSO-d₆) δ −136.7 (d, J_{FF}=22.5 Hz, 2F), −137.8 (d, J_{FF}=22.5 Hz, 2F). HR-APCI m/z calcd for C₃₄H₂₉F₄N₄O₄S [M+H]=665.1874, found 665.1840.

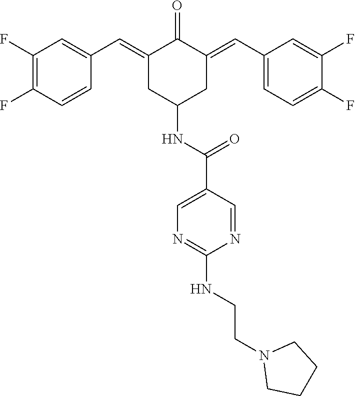 [SMILES: O=C1/C(=C/c2ccc(F)c(F)c2)CC(NC(=O)c2cnc(NCCN3CCCC3)nc2)C/C1=C\c1ccc(F)c(F)c1]

### N-(3,5-Bis((E)-3,4-difluorobenzylidene)-4-oxocyclohexyl)-2-((2-(pyrrolidin-1-yl)ethyl)amino)pyrimi-dine-5-carboxamide (JCSO90)

¹H NMR (500 MHz, DMSO-d₆) δ 8.70 (s, 2H), 8.49 (d, J=6.2 Hz, 1H), 7.69-7.40 (m, 9H), 4.10-4.02 (m, 1H), 3.41 (t, J=7.1 Hz, 2H), 3.18 (br d, J=13.3 Hz, 2H), 3.01 (dd, J=15.0, 8.0 Hz, 2H), 2.57 (t, J=6.8 Hz, 2H), 2.47 (t, J=6.4 Hz, 4H), 1.69-1.64 (m, 4H).

¹³C NMR (126 MHz, DMSO-d₆) δ 188.0, 164.1, 163.2, 158.3 (2C), 150.5 (dd, J_{CF}=249.4 Hz, J_{C-CF}=12.6 Hz, 2C), 149.7 (dd, J_{CF}=249.4 Hz, J_{C-CF}=12.6 Hz, 2C), 135.6 (2C), 135.0 (2C), 132.1 (dd, J_{C-C-CF}=6.3 Hz, J_{C-C-CF}=3.7 Hz, 2C), 128.0 (dd, J_{C-C-CF}=6.3 Hz, J_{C-C-CF}=3.7 Hz, 2C), 119.5 (d, J_{C-CF}=17.6 Hz, 2C), 118.2 (d, J_{C-CF}=17.6 Hz, 2C), 116.3, 54.9, 54.0, 45.2, 40.3, 33.5, 23.5.

¹⁹F NMR (376 MHz, DMSO-d₆) δ −136.9 (d, J_{FF}=22.5 Hz, 2F), −137.9 (d, J_{FF}=22.5 Hz, 2F).

HR-APCI m/z calcd for C₃₁H₃₀F₄N₅O₂ [M+H]=580.2330, found 580.2306.

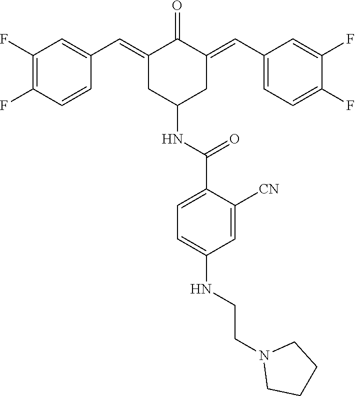 [SMILES: N#Cc1cc(NCCN2CCCC2)ccc1C(=O)NC1C/C(=C\c2ccc(F)c(F)c2)C(=O)/C(=C/c2ccc(F)c(F)c2)C1]

### N-(3,5-Bis((E)-3,4-difluorobenzylidene)-4-oxocyclohexyl)-2-cyano-4-((2-(pyrrolidin-1-yl)ethyl)ami-no)benzamide (JCSO91)

¹H NMR (500 MHz, CDCl₃) δ 7.80 (s, 2H), 7.60 (d, J=8.7 Hz, 1H), 7.30-7.15 (m, 6H), 6.78 (s, 1H), 6.75 (d, J=7.2 Hz, 1H), 6.43 (d, J=7.2 Hz, 1H), 5.32 (br s, 1H), 4.53-4.47 (m, 1H), 3.28-3.21 (m, 4H), 3.09 (dd, J=16.1, 8.4 Hz, 2H), 2.83 (t, J=6.0 Hz, 2H), 2.65 (t, J=6.3 Hz, 4H), 1.87-1.84 (m, 4H).

¹³C NMR (126 MHz, CDCl₃) δ 187.9, 164.7, 150.7 (dd, J_{CF}=253.2 Hz, J_{C-CF}=12.6 Hz, 2C), 150.5 (dd, J_{CF}=249.4 Hz, J_{C-CF}=12.6 Hz, 2C), 150.3, 138.1 (2C), 132.8 (2C), 132.3 (dd, J_{C-C-CF}=6.3 Hz, J_{C-C-CF}=3.7 Hz, 2C), 131.0, 127.1 (dd, J_{C-C-CF}=6.3 Hz, J_{C-C-CF}=3.7 Hz, 2C), 124.3, 119.1 (d, J_{C-CF}=17.6 Hz, 2C), 118.7, 117.7 (d, J_{C-CF}=17.6 Hz, 2C), 116.3, 116.1, 111.3, 54.1, 53.9, 45.2, 41.2, 33.7, 23.6.

¹⁹F NMR (376 MHz, CDCl₃) δ −135.2 (d, J_{FF}=18.8 Hz, 2F), −136.6 (d, J_{FF}=22.5 Hz, 2F).

HR-APCI m/z calcd for C₃₄H₃₁F₄N₄O₂ [M+H]=603.2377, found 603.2395.

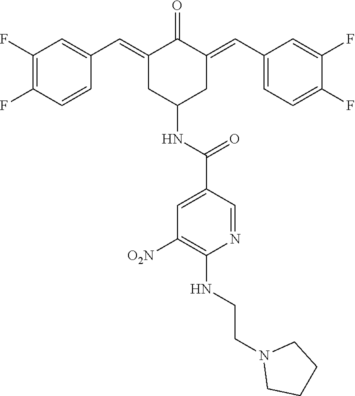 [SMILES: O=C1/C(=C/c2ccc(F)c(F)c2)CC(NC(=O)c2cnc(NCCN3CCCC3)c([N+](=O)[O-])c2)C/C1=C\c1ccc(F)c(F)c1]

### N-(3,5-Bis((E)-3,4-difluorobenzylidene)-4-oxocyclohexyl)-5-nitro-6-((2-(pyrrolidin-1-yl)ethyl)ami-no)nicotinamide (JCSO92)

¹H NMR (500 MHz, CDCl₃) δ 8.82 (d, J=2.5 Hz, 1H), 8.80 (br s, 1H) 8.72 (d, J=2.3 Hz, 1H), 7.76 (s, 2H), 7.27-7.15 (m, 6H), 6.67 (d, J=7.3 Hz, 1H), 4.53-4.45 (m, 1H), 3.78-3.74 (m, 2H), 3.29 (br d, J=15.8 Hz, 2H), 3.08 (dd, J=17.0, 8.5 Hz, 2H), 2.80 (t, J=6.2 Hz, 2H), 2.62 (t, J=5.9 Hz, 4H), 1.89-1.71 (m, 4H).

¹³C NMR (126 MHz, CDCl₃) δ 187.7, 163.7, 155.3, 153.2, 150.6 (dd, J_{CF}=253.2 Hz, J_{C-CF}=12.6 Hz, 2C), 150.2 (dd, J_{CF}=249.4 Hz, J_{C-CF}=12.6 Hz, 2C), 137.7, 133.6 (2C), 132.6 (2C), 132.0 (dd, J_{C-C-CF}=6.3 Hz, J_{C-C-CF}=3.7 Hz, 2C), 127.1 (dd, J_{C-C-CF}=6.3 Hz, J_{C-C-CF}=3.7 Hz, 2C), 126.6, 118.8 (d, J_{C-CF}=17.6 Hz, 2C), 117.6 (d, J_{C-CF}=17.6 Hz, 2C), 117.4, 54.0, 53.9, 44.9, 40.2, 33.7, 23.5.

¹⁹F NMR (376 MHz, CDCl₃) δ −134.8 (d, J_{FF}=18.8 Hz, 2F), −136.3 (d, J_{FF}=18.8 Hz, 2F).

HR-APCI m/z calcd for C₃₂H₃₀F₄N₅O₄ [M+H]=624.2202, found 624.2228.

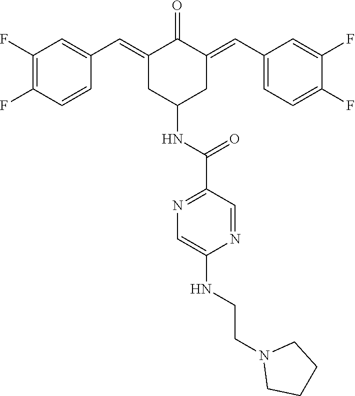 [SMILES: O=C1/C(=C/c2ccc(F)c(F)c2)CC(NC(=O)c2cnc(NCCN3CCCC3)cn2)C/C1=C\c1ccc(F)c(F)c1]

### N-(3,5-Bis((E)-3,4-difluorobenzylidene)-4-oxocyclohexyl)-5-((2-(pyrrolidin-1-yl)ethyl)amino)pyr-azine-2-carboxamide (JCSO93)

¹H NMR (500 MHz, CDCl₃) δ 8.70 (s, 1H), 7.78 (s, 2H), 7.70 (s, 1H), 7.56 (d, J=8.2 Hz, 1H), 7.32-7.14 (m, 6H), 5.94 (d, J=7.3 Hz, 1H), 4.46-4.40 (m, 1H), 3.50-3.47 (m, 2H), 3.25 (br d, J=14.4 Hz, 2H), 3.01 (dd, J=14.4, 9.1 Hz, 2H), 2.75 (t, J=5.9 Hz, 2H), 2.57 (t, J=5.9 Hz, 4H), 1.89-1.71 (m, 4H).

¹³C NMR (126 MHz, CDCl₃) δ 187.8, 163.7, 155.6, 150.4 (dd, J_{CF}=253.2 Hz, J_{C-CF}=12.6 Hz, 2C), 150.0 (dd, J_{CF}=249.4 Hz, J_{C-CF}=12.6 Hz, 2C), 142.9, 137.4 (2C), 132.8 (2C), 132.4, 132.1 (dd, J_{C-C-CF}=6.3 Hz, J_{C-C-CF}=3.7 Hz, 2C), 129.8, 126.8 (dd, J_{C-C-CF}=6.3 Hz, J_{C-C-CF}=3.7 Hz, 2C), 118.8 (d, J_{C-CF}=16.3 Hz, 2C), 117.6 (d, J_{C-CF}=16.3 Hz, 2C), 54.0, 53.6, 43.8, 39.4, 33.8, 23.3.

¹⁹F NMR (376 MHz, CDCl₃) δ −135.3 (d, J_{FF}=18.8 Hz, 2F), −136.6 (d, J_{FF}=18.8 Hz, 2F).

HR-APCI m/z calcd for C₃₁H₃₀F₄N₅O₂ [M+H]=580.2306, found 580.2330.

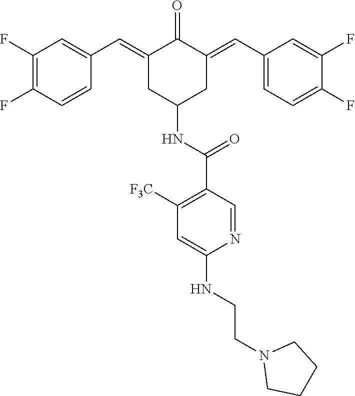 [SMILES: O=C1/C(=C/c2ccc(F)c(F)c2)CC(NC(=O)c2cnc(NCCN3CCCC3)cc2C(F)(F)F)C/C1=C\c1ccc(F)c(F)c1]

### N-(3,5-Bis((E)-3,4-difluorobenzylidene)-4-oxocyclohexyl)-6-((2-(pyrrolidin-1-yl)ethyl)amino)-4-(trifluoromethyl)nicotinamide (JCSO94)

¹H NMR (500 MHz, CDCl₃) δ 8.18 (s, 1H), 7.81 (s, 2H), 7.29-7.18 (m, 6H), 6.58 (s, 1H), 6.32 (s, 1H), 6.02 (d, J=7.2 Hz, 1H), 4.51-4.47 (m, 1H), 3.55-3.49 (m, 2H), 3.22 (br d, J=15.2 Hz, 2H), 3.12 (dd, J=15.7, 7.0 Hz, 2H), 2.85 (t, J=5.7 Hz, 2H), 2.72 (t, J=5.9 Hz, 4H), 1.96-1.81 (m, 4H).

¹³C NMR (126 MHz, CDCl₃) δ 187.6, 165.9, 159.2, 150.6 (dd, J_{CF}=254.5 Hz, J_{C-CF}=12.6 Hz, 2C), 150.2 (dd, J_{CF}=249.4 Hz, J_{C-CF}=12.6 Hz, 2C), 149.5, 138.1 (2C), 136.1 (q, J_{C-F}=32.7 Hz, C), 132.3 (2C), 132.0 (dd, J_{C-C-CF}=6.3 Hz, J_{C-C-CF}=3.7 Hz, 2C), 127.0 (dd, J_{C-C-CF}=6.3 Hz, J_{C-C-CF}=3.7 Hz, 2C), 123.5, 121.3, 118.8 (d, J_{C-CF}=16.3 Hz, 2C), 117.6 (d, J_{C-CF}=16.3 Hz, 2C), 117.4, 54.4, 53.8, 44.7, 39.5, 33.0, 23.4.

¹⁹F NMR (376 MHz, CDCl₃) δ −61.4 (s, 3F), −135.0 (d, J_{FF}=18.8 Hz, 2F), −136.5 (d, J_{FF}=18.8 Hz, 2F).

HR-APCI m/z calcd for C₃₃H₃₀F₇N₄O₂ [M+H]=647.2230, found 647.2251.

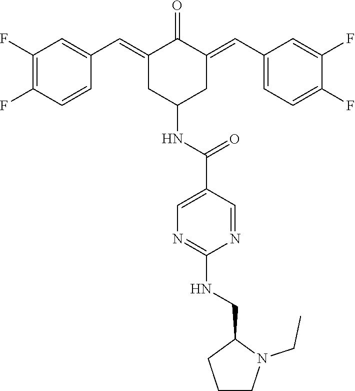 [SMILES: CCN1CCC[C@H]1CNc1ncc(C(=O)NC2C/C(=C\c3ccc(F)c(F)c3)C(=O)/C(=C/c3ccc(F)c(F)c3)C2)cn1]

### N-(3,5-Bis((E)-3,4-difluorobenzylidene)-4-oxocyclohexyl)-2-((((S)-1-ethylpyrrolidin-2-yl)methyl)-amino)pyrimidine-5-carboxamide (JCS136)

¹H NMR (500 MHz, CDCl₃) δ 8.71 (s, 2H), 7.73 (s, 2H), 7.30-7.16 (m, 6H), 6.70 (br s, 2H), 4.44-4.40 (m, 1H), 3.78-3.65 (m, 1H), 3.55-3.34 (m, 2H), 3.26 (br d, J=12.6 Hz, 2H), 3.09-2.98 (m, 4H), 2.62-2.42 (m, 2H), 2.10-1.94 (m, 1H), 1.91-1.66 (m, 3H), 1.19 (t, J=7.2 Hz, 3H).

¹³C NMR (126 MHz, CDCl₃) δ 187.8, 164.0, 163.4, 158.3 (2C), 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 137.5 (2C), 133.0 (2C), 132.3 (dd, J_{C-C-CF}=6.3 Hz, J_{C-C-CF}=3.7 Hz, 2C), 127.3 (dd, J_{C-C-CF}=6.3 Hz, J_{C-C-CF}=3.7 Hz, 2C), 119.0 (d, J_{C-CF}=17.6 Hz, 2C), 117.7 (d, J_{C-CF}=17.6 Hz, 2C), 117.2, 64.4, 53.9, 49.2, 44.9, 43.0, 33.8, 28.1, 23.0, 12.9.

¹⁹F NMR (376 MHz, CDCl₃) δ −135.2 (d, J_{FF}=26.3 Hz, 2F), −136.6 (d, J_{FF}=26.3 Hz, 2F).

HR-APCI m/z calcd for C₃₂H₃₂F₄N₅O₂ [M+H]=594.2534, found 594.2486.

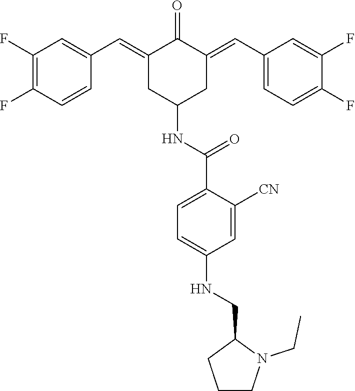 [SMILES: CCN1CCC[C@H]1CNc1ccc(C(=O)NC2C/C(=C\c3ccc(F)c(F)c3)C(=O)/C(=C/c3ccc(F)c(F)c3)C2)c(C#N)c1]

### N-(3,5-Bis((E)-3,4-difluorobenzylidene)-4-oxocyclohexyl)-2-cyano-4-((((S)-1-ethylpyrrolidin-2-yl)methyl)amino)benzamide (JCS139)

¹H NMR (500 MHz, CDCl₃) δ 7.81 (s, 2H), 7.61 (d, J=8.2 Hz, 1H), 7.32-7.19 (m, 7H), 6.99 (s, 1H), 6.79 (d, J=8.3 Hz, 1H), 5.41 (br s, 1H), 4.61-4.41 (m, 1H), 3.78 (t, J=14.1 Hz, 2H), 3.29-3.07 (m, 5H), 2.85-2.72 (m, 2H), 2.33-2.20 (m, 2H), 2.02-1.93 (m, 1H), 1.85-1.69 (m, 3H), 1.15 (t, J=7.0 Hz, 3H).

¹³C NMR (126 MHz, CDCl₃) δ 188.1, 167.0, 154.2, 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 136.8 (2C), 134.8, 134.0 (2C), 132.7, 132.3 (dd, J_{C-C-CF}=6.3 Hz, J_{C-C-CF}=3.7 Hz, 2C), 127.1 (dd, J_{C-C-CF}=6.3 Hz, J_{C-C-CF}=3.7 Hz, 2C), 125.3, 119.0 (d, J_{C-CF}=17.6 Hz, 2C), 118.1, 117.6 (d, J_{C-CF}=17.6 Hz, 2C), 116.4, 106.1, 62.2, 53.6, 48.0, 46.1, 44.1, 31.5, 28.7, 22.8, 13.9.

¹⁹F NMR (376 MHz, CDCl₃) δ −135.5 (d, J_{FF}=26.3 Hz, 2F), −136.7 (d, J_{FF}=26.3 Hz, 2F).

HR-APCI m/z calcd for C₃₅H₃₁F₄N₄O₂ [M−H]=615.2397, found 615.2388.

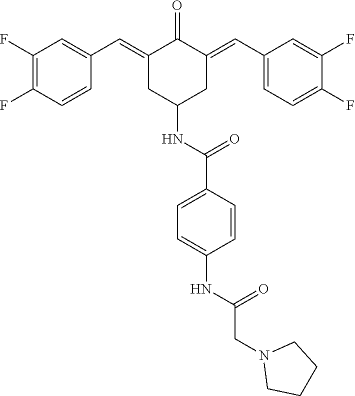 [SMILES: O=C(CN1CCCC1)Nc1ccc(C(=O)NC2C/C(=C\c3ccc(F)c(F)c3)C(=O)/C(=C/c3ccc(F)c(F)c3)C2)cc1]

### N-(3,5-bis((E)-3,4-difluorobenzylidene)-4-oxocyclohexyl)-4-(2-(pyrrolidin-1-yl)acetamido)benzamide (JCSO46)

To the mixture of the tert-butyl (4-oxocyclohexyl)carbamate (213.28 mg, 1 mmol, 1.0 equiv.) and ethanol (1.0 mL) in a round bottom flask added drop-wise 20% aqueous sodium hydroxide (1.5 mL) and stirred for five minutes. To this mixture was added 3,4-difluorobenzaldehyde (355.3 mg, 2.5 mmol, 2.5 equiv.). The reaction mixture was then allowed to stir at room temperature for 5 h. After 5 h the yellow precipitate thus obtained was filtered, washed with water, cold ethanol and dried to get pure product (360 mg, 78% yield).

Trifluoroacetic acid (0.5 ml) was added to a solution of tert-butyl (3,5-bis((E)-3,4-difluorobenzylidene)-4-oxocyclohexyl)carbamate (230.7 mg, 0.5 mmol) in methylene chloride (5.0 ml) at room temperature and stirred overnight at room temperature. Then, the solvent of the reaction solution was distilled off under reduced pressure and the resulting residue was poured into a 1N-aqueous sodium hydroxide solution and extracted with ethyl acetate. The organic layer was washed with a saturated aqueous sodium chloride solution and then dried over anhydrous magnesium sulfate. The solvent was distilled off under reduced pressure to obtain 4-amino-2,6-bis((E)-3,4-difluorobenzylidene)cyclohexan-1-one.

The mixture of 4-amino-2,6-bis((E)-3,4-difluorobenzylidene)cyclohexan-1-one (180.7 mg, 0.5 mmol, 1.0 equiv.) and anhydrous diisopropylethylamine (261.2 μL, 1.5 mmol, 3.0 equiv.) in THF was maintained at 0° C. (ice bath). To this cooled mixture, 4-(2-(pyrrolidin-1-yl)acetamido)benzoic acid hydrochloride (142.0 mg, 0.5 mmol, 1.0 equiv.) in 2.0 mL THF was added drop wise followed by and TBTU (240.8 mg, 0.75 mmol, 1.5 equiv.). After the complete addition of 4-(2-(pyrrolidin-1-yl)acetamido)benzoic acid hydrochloride the reaction mixture was slowly warmed up to room temperature and stirred overnight. After completion of the reaction solvent was evaporated and the residue was stirred in sat aqueous NaHCO₃ for 5 min. The mixture was extracted with ethyl acetate three times. The organic layer was washed with a saturated aqueous sodium chloride solution and then dried over anhydrous sodium sulfate. The solvent was evaporated, followed by flash chromatography (gradient elution 20% methanol/EtOAc-75% methanol/EtOAc) to give desired product JCSO46 (147.7 mg, 60% yield) as yellow solid.

¹H NMR (500 MHz, CDCl₃) δ 9.29 (s, 1H), 7.82 (s, 2H), 7.67-7.63 (m, 6H), 7.26-7.17 (m, 6H), 6.11 (d, J=7.3 Hz, 1H), 4.54-4.48 (m, 1H), 3.29-3.25 (m, 4H), 3.07 (dd, J=17.0, 8.1 Hz, 2H), 2.70 (t, J=6.3 Hz, 4H), 1.87 (t, J=6.3 Hz, 4H).

¹³C NMR (126 MHz, CDCl₃) δ 188.0, 169.6, 166.5, 150.8 (dd, J_{CF}=253.2 Hz, J_{C-CF}=12.6 Hz, 2C), 150.3 (dd, J_{CF}=249.4 Hz, J_{C-CF}=12.6 Hz, 2C), 141.0, 137.9 (2C), 132.8 (2C), 132.2 (dd, J_{C-C-CF}=6.3 Hz, J_{C-C-CF}=3.7 Hz, 2C), 129.2 (2C), 127.2, 127.1 (dd, J_{C-C-CF}=6.3 Hz, J_{C-C-CF}=3.7 Hz, 2C), 119.1 (d, J_{C-CF}=17.6 Hz, 2C), 119.0 (2C), 117.8 (d, J_{C-CF}=17.6 Hz, 2C), 59.8, 54.8, 44.8, 33.8, 24.2.

¹⁹F NMR (376 MHz, CDCl₃) δ −135.0 (d, J_{FF}=22.5 Hz, 2F), −136.5 (d, J_{FF}=18.8 Hz, 2F).

HR-APCI m/z calcd for C₃₃H₃₀F₄N₃O₃ [M+H]=592.2217, found 592.2199.

### Synthesis of 4-(2-(pyrrolidin-1-yl)acetamido)benzoic acid

To a round bottom flask equipped with a stir-bar was added a solution of ethyl 4-aminobenzoate (0.33 g, 2.0 mmol, 1.0 equiv), dimethylformamide (DMF, 10 mL), 2-(pyrrolidin-1-yl)acetic acid (0.25 g, 2.0 mmol, 1.0 equiv), HATU (0.83 g, 2.2 mmol, 1.1 equiv) and diisopropylethylamine (0.69 mL, 4.0 mmol, 2.0 equiv). The mixture was stirred at 21° C. for 16 h, after which time ethyl acetate (20 mL) and water (30 mL) were added. The organic layer was washed with water three times and then dried over sodium sulfate. The pure product was obtained by flash chromatography on silica gel (gradient elution 10% methanol/EtOAc-50% methanol/EtOAc) to give the desired product, ethyl 4-(2-(pyrrolidin-1-yl)acetamido)benzoate (0.38 g, 70% yield), as a yellow solid.

Ethyl 4-(2-(pyrrolidin-1-yl)acetamido)benzoate (0.290 g, 1.0 mmol, 1.0 equiv) was dissolved in 2.5 mL ethanol and added to a solution of sodium hydroxide (0.2 g) in 2.5 mL water. The mixture was heated under reflux for 2 h. The ethanol was removed in vacuo and the aqueous solution was acidified with cone. HCl at 5° C. The solid was collected, treated with cold water, filtered and dried at 55-60° C. in vacuo to give 4-(2-(propylamino)ethoxy)benzoic acid hydrochloride as white sold (0.25 g, 90% yield) which was pure enough to be used for next step.

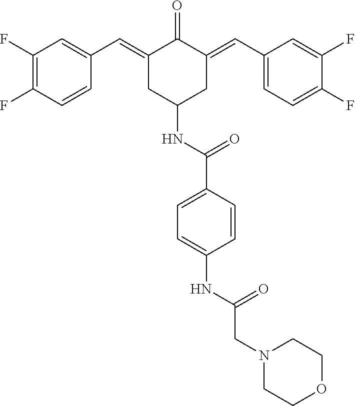 [SMILES: O=C(CN1CCOCC1)Nc1ccc(C(=O)NC2C/C(=C\c3ccc(F)c(F)c3)C(=O)/C(=C/c3ccc(F)c(F)c3)C2)cc1]

### N-(3,5-bis((E)-3,4-difluorobenzylidene)-4-oxocyclohexyl)-4-(2-morpholinoacetamido)benzamide (JCSO47)

¹H NMR (500 MHz, CDCl₃) δ 9.21 (s, 1H), 7.80 (s, 2H), 7.73-7.55 (m, 4H), 7.27-7.16 (m, 6H), 6.22 (d, J=7.3 Hz, 1H), 4.53-4.46 (m, 1H), 3.78 (t, J=4.6 Hz, 4H), 3.26 (bd, J=15.6 Hz, 2H), 3.14 (s, 2H), 3.12-3.01 (m, 2H), 2.62 (t, J=4.6 Hz, 4H).

¹³C NMR (126 MHz, CDCl₃) δ 187.9, 168.4, 166.4, 150.8 (dd, J_{CF}=253.2 Hz, J_{C-CF}=12.6 Hz, 2C), 150.3 (dd, J_{CF}=249.4 Hz, J_{C-CF}=12.6 Hz, 2C), 140.7, 138.0 (2C), 132.8 (2C), 132.2 (dd, J_{C-C-CF}=6.3 Hz, J_{C-C-CF}=3.7 Hz, 2C), 129.5 (2C), 128.2, 127.2 (dd, J_{C-C-CF}=6.3 Hz, J_{C-C-CF}=3.7 Hz, 2C), 119.1 (d, J_{C-CF}=17.6 Hz, 2C), 119.0 (2C), 117.8 (d, J_{C-CF}=17.6 Hz, 2C), 67.1, 62.5, 53.9, 44.8, 33.8.

¹⁹F NMR (376 MHz, CDCl₃) δ −135.0 (d, J_{FF}=22.5 Hz, 2F), −136.4 (d, J_{FF}=22.5 Hz, 2F).

HR-APCI m/z calcd for C₃₃H₃₀F₄N₃O₄ [M+H]=608.2166, found 608.2142.

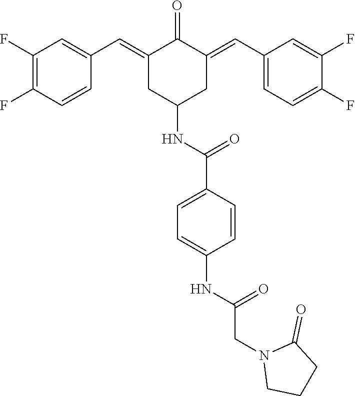 [SMILES: O=C(CN1CCCC1=O)Nc1ccc(C(=O)NC2C/C(=C\c3ccc(F)c(F)c3)C(=O)/C(=C/c3ccc(F)c(F)c3)C2)cc1]

### N-(3,5-bis((E)-3,4-difluorobenzylidene)-4-oxocyclohexyl)-4-(2-(2-oxopyrrolidin-1-yl)acetamido)benzamide (JCSO48)

¹H NMR (500 MHz, CDCl₃) δ 8.83 (s, 1H), 7.81 (s, 2H), 7.60-7.48 (m, 4H), 7.28-7.18 (m, 6H), 6.19 (d, J=7.3 Hz, 1H), 4.53-4.46 (m, 1H), 4.05 (s, 2H), 3.61 (t, J=7.1 Hz, 2H), 3.27 (bd, J=15.2 Hz, 2H), 3.06 (dd, J=15.9, 8.5 Hz, 2H), 2.49 (t, J=8.1 Hz, 2H), 2.18-2.12 (m, 2H).

¹³C NMR (126 MHz, CDCl₃) δ 187.9, 177.0, 166.8, 166.6, 150.8 (dd, J_{CF}=253.2 Hz, J_{C-CF}=12.6 Hz, 2C), 150.3 (dd, J_{CF}=249.4 Hz, J_{C-CF}=12.6 Hz, 2C), 141.0, 138.0 (2C), 132.8 (2C), 132.2 (dd, J_{C-C-CF}=6.3 Hz, J_{C-C-CF}=3.7 Hz, 2C), 129.5 (2C), 128.0, 127.2 (dd, J_{C-C-CF}=6.3 Hz, J_{C-C-CF}=3.7 Hz, 2C), 119.3 (2C), 119.0 (d, J_{C-CF}=17.6 Hz, 2C), 117.8 (d, J_{C-CF}=17.6 Hz, 2C), 49.3, 49.2, 44.8, 33.8, 30.5, 18.2.

¹⁹F NMR (376 MHz, CDCl₃) δ −135.0 (d, J_{FF}=18.8 Hz, 2F), −136.4 (d, J_{FF}=22.5 Hz, 2F).

HR-APCI m/z calcd for C₃₃H₂₈F₄N₃O₄ [M+H]=606.2010, found 606.1991.

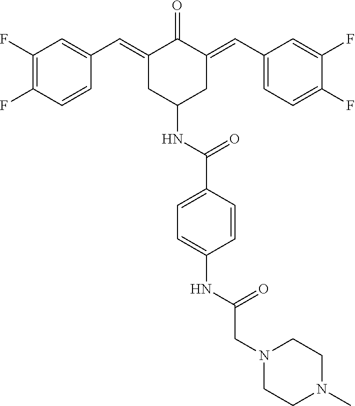 [SMILES: CN1CCN(CC(=O)Nc2ccc(C(=O)NC3C/C(=C\c4ccc(F)c(F)c4)C(=O)/C(=C/c4ccc(F)c(F)c4)C3)cc2)CC1]

### N-(3,5-bis((E)-3,4-difluorobenzylidene)-4-oxocyclohexyl)-4-(2-(4-methylpiperazin-1-yl)acetamido)benzamide (JCS050)

¹H NMR (500 MHz, CDCl₃) δ 9.29 (s, 1H), 7.76 (d, J=2.0 Hz, 2H), 7.67 (d, J=8.8 Hz, 2H), 7.58 (d, J=8.7 Hz, 2H), 7.25-7.13 (m, 6H), 6.45 (d, J=7.3 Hz, 1H), 4.49-4.43 (m, 1H), 3.25 (bd, J=15.3 Hz, 2H), 3.11 (s, 2H), 3.05 (dd, J=15.9, 8.7 Hz, 2H), 2.63 (t, J=5.1 Hz, 2H), 2.49 (t, J=5.1 Hz, 2H), 2.31 (s, 3H).

¹³C NMR (126 MHz, CDCl₃) δ 187.9, 168.8, 166.4, 150.7 (dd, J_{CF}=253.2 Hz, J_{C-CF}=12.6 Hz, 2C), 150.2 (dd, J_{CF}=249.4 Hz, J_{C-CF}=12.6 Hz, 2C), 140.7, 137.8 (2C), 132.9 (2C), 132.2 (dd, J_{C-C-CF}=6.3 Hz, J_{C-C-CF}=3.7 Hz, 2C), 129.3 (2C), 128.2, 127.1 (dd, J_{C-C-CF}=6.3 Hz, J_{C-C-CF}=3.7 Hz, 2C), 119.0 (d, J_{C-CF}=17.6 Hz, 2C), 118.9 (2C), 117.8 (d, J_{C-CF}=17.6 Hz, 2C), 61.9, 55.3, 53.5, 46.0, 44.8, 33.7.

¹⁹F NMR (376 MHz, CDCl₃) δ 131.1 (d, J_{FF}=30.0 Hz, 2F), 129.7 (d, J_{FF}=30.0 Hz, 2F).

HR-APCI m/z calcd for C₃₄H₃₃F₄N₄O₃ [M+H]=621.2483, found 621.2464.

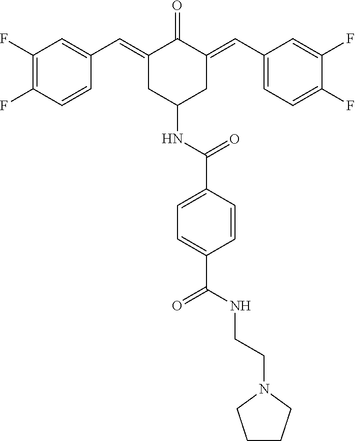 [SMILES: O=C1/C(=C/c2ccc(F)c(F)c2)CC(NC(=O)c2ccc(C(=O)NCCN3CCCC3)cc2)C/C1=C\c1ccc(F)c(F)c1]

### N1-(3,5-bis((E)-3,4-difluorobenzylidene)-4-oxocyclohexyl)-N4-(2-(pyrrolidin-1-yl)ethyl)terephthalamide (JCS076)

¹H NMR (500 MHz, CDCl₃) δ 9.01 (t, J=5.7 Hz, 1H), 8.17 (d, J=8.1 Hz, 2H), 7.90 (s, 2H), 7.86 (d, J=8.2 Hz, 2H), 7.41-7.31 (m, 6H), 7.05 (d, J=7.2 Hz, 1H), 4.59-4.52 (m, 1H), 4.04-3.96 (m, 4H), 3.48-3.43 (m, 2H), 3.42 (bd, J=15.8 Hz, 2H), 3.22 (dd, J=15.4, 9.7 Hz, 2H), 3.02 (t, J=8.9 Hz, 2H), 2.41-2.18 (m, 4H). ¹³C NMR (126 MHz, CDCl₃) δ 187.8, 166.8, 166.5, 150.7 (dd, J_{CF}=254.5 Hz, J_{C-CF}=12.6 Hz, 2C), 150.3 (dd, J_{CF}=249.4 Hz, J_{C-CF}=12.6 Hz, 2C), 137.8, 136.8, 136.5, 132.9 (2C), 132.2 (dd, J_{C-C-CF}=6.3 Hz, J_{C-C-CF}=3.7 Hz, 2C), 127.8 (2C), 127.3 (2C), 127.2 (dd, J_{C-C-CF}=6.3 Hz, J_{C-C-CF}=3.7 Hz, 2C), 119.0 (d, J_{C-CF}=17.6 Hz, 2C), 117.8 (d, J_{C-CF}=17.6 Hz, 2C), 55.7, 54.5, 45.2, 37.0, 33.8, 23.5.

¹⁹F NMR (376 MHz, CDCl₃) δ 131.0 (d, J_{FF}=30.0 Hz, 2F), −129.6 (d, J_{FF}=30.0 Hz, 2F).

HR-APCI m/z calcd for C₃₄H₃₂F₄N₃O₃ [M+H]=606.2374, found 606.2386.

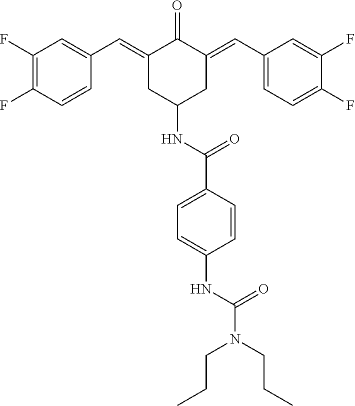 [SMILES: CCCN(CCC)C(=O)Nc1ccc(C(=O)NC2C/C(=C\c3ccc(F)c(F)c3)C(=O)/C(=C/c3ccc(F)c(F)c3)C2)cc1]

### N-(3,5-bis((E)-3,4-difluorobenzylidene)-4-oxocyclohexyl)-4-(3,3-dipropylureido)benzamide (JCS051)

To the mixture of the tert-butyl (4-oxocyclohexyl)carbamate (213.28 mg, 1 mmol, 1.0 equiv.) and ethanol (1.0 mL) in a round bottom flask added drop-wise 20% aqueous sodium hydroxide (1.5 mL) and stirred for five minutes. To this mixture was added 3,4-difluorobenzaldehyde (355.3 mg, 2.5 mmol, 2.5 equiv.). The reaction mixture was then allowed to stir at room temperature for 5 h. After 5 h the yellow precipitate thus obtained was filtered, washed with water, cold ethanol and dried to get pure product (360 mg, 78% yield).

Trifluoroacetic acid (0.5 ml) was added to a solution of tert-butyl (3,5-bis((E)-3,4-difluorobenzylidene)-4-oxocyclohexyl)carbamate (230.7 mg, 0.5 mmol) in methylene chloride (5.0 ml) at room temperature and stirred overnight at room temperature. Then, the solvent of the reaction solution was distilled off under reduced pressure and the resulting residue was poured into a 1N-aqueous sodium hydroxide solution and extracted with ethyl acetate. The organic layer was washed with a saturated aqueous sodium chloride solution and then dried over anhydrous magnesium sulfate. The solvent was distilled off under reduced pressure to obtain 4-amino-2,6-bis((E)-3,4-difluorobenzylidene)cyclohexan-1-one.

The mixture of 4-amino-2,6-bis((E)-3,4-difluorobenzylidene)cyclohexan-1-one (180.7 mg, 0.5 mmol, 1.0 equiv.) and anhydrous diisopropylethylamine (261.2 μL, 1.5 mmol, 3.0 equiv.) in THF was maintained at 0° C. (ice bath). To this cooled mixture, 4-(3,3-dipropylureido)benzoic acid hydrochloride (150.3 mg, 0.5 mmol, 1.0 equiv.) in 2.0 mL THF was added drop wise followed by and TBTU (240.8 mg, 0.75 mmol, 1.5 equiv.). After the complete addition of 4-(3,3-dipropylureido)benzoic acid hydrochloride the reaction mixture was slowly warmed up to room temperature and stirred overnight. After completion of the reaction solvent was evaporated and the residue was stirred in sat aqueous NaHCO₃ for 5 min. The mixture was extracted with ethyl acetate three times. The organic layer was washed with a saturated aqueous sodium chloride solution and then dried over anhydrous sodium sulfate. The solvent was evaporated, followed by flash chromatography (gradient elution 20% methanol/EtOAc-75% methanol/EtOAc) to give desired product JCS051 (182 mg, 60% yield) as yellow solid.

¹H NMR (500 MHz, DMSO-d₆) δ 8.40 (d, J=6.6 Hz, 1H), 8.36 (s, 1H), 7.78-7.70 (br d, J=8.2 Hz, 2H), 7.70-7.60 (m, 4H), 7.59-7.49 (m, 4H), 7.46-7.39 (m, 2H), 4.11-4.04 (m, 1H), 3.25 (t, J=7.0 Hz, 4H), 3.19 (br d, J=15.0 Hz, 2H), 3.01 (dd, J=15.0, 8.1 Hz, 2H), 1.51 (sextet, J=7.4 Hz, 4H), 0.85 (t, J=7.4 Hz, 6H).

¹³C NMR (126 MHz, DMSO-d₆) δ 187.6, 165.7, 154.4, 149.6 (dd, J_{CF}=250.7 Hz, J_{C-CF}=12.6 Hz, 2C), 149.3 (dd, J_{CF}=246.9 Hz, J_{C-CF}=12.6 Hz, 2C), 143.7, 135.1 (2C), 134.7 (2C), 132.7 (dd, J_{C-C-CF}=6.3 Hz, J_{C-C-CF}=3.7 Hz, 2C), 127.7 (2C), 127.6 (dd, J_{C-C-CF}=6.3 Hz, J_{C-C-CF}=3.7 Hz, 2C), 126.7, 119.1 (d, J_{C-CF}=17.6 Hz, 2C), 118.4 (2C), 117.8 (d, J_{C-CF}=17.6 Hz, 2C), 48.0, 45.0, 33.1, 21.2, 11.1. ¹⁹F NMR (376 MHz, DMSO-d₆) δ −136.9 (d, J_{FF}=22.5 Hz, 2F), −137.9 (d, J_{FF}=18.8 Hz, 2F).

HR-APCI m/z calcd for C₃₄H₃₄F₄N₃O₃ [M+H]=608.2530, found 608.2517.

### Synthesis of 4-(3,3-dipropylureido)benzoic acid

To a round bottom flask equipped with a stir-bar was added a solution of methyl 4-isocyanatobenzoate (0.35 g, 2.0 mmol, 1.0 equiv), THF (5 mL). Then, dipropylamine (0.22 g, 2.2 mmol, 1.1 equiv) was added in bulk and the reaction mixture was stirred at 21° C. overnight. The solution was concentrated to a slurry, diluted with ether, and filtered to provide methyl 4-(3,3-dipropylureido)benzoate as a white solid (0.50 g, 90%) which was pure enough to be used for next step.

Methyl 4-(3,3-dipropylureido)benzoate (0.27 g, 1.0 mmol, 1.0 equiv) was dissolved in 2.5 mL ethanol and added to a solution of sodium hydroxide (0.2 g) in 2.5 mL water. The mixture was heated under reflux for 2 h. The ethanol was removed in vacuo and the aqueous solution was acidified with cone. HCl at 5° C. The solid was collected, treated with cold water, filtered and dried at 55-60° C. in vacuo to give 4-(3,3-dipropylureido)benzoic acid hydrochloride as white solid (0.27 g, 90% yield) which was pure enough to be used for next step.

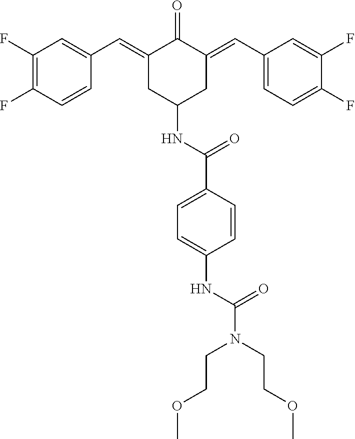 [SMILES: COCCN(CCOC)C(=O)Nc1ccc(C(=O)NC2C/C(=C\c3ccc(F)c(F)c3)C(=O)/C(=C/c3ccc(F)c(F)c3)C2)cc1]

### N-(3,5-bis((E)-3,4-difluorobenzylidene)-4-oxocyclohexyl)-4-(3,3-bis(2-methoxyethyl)ureido)benzamide (JCS052)

¹H NMR (500 MHz, DMSO-d₆) δ 8.57 (s, 1H), 8.41 (d, J=6.6 Hz, 1H), 7.74 (d, J=8.7 Hz, 2H), 7.69-7.60 (m, 4H), 7.56-7.38 (m, 6H), 4.12-4.04 (m, 1H), 3.53-3.48 (m, 8H), 3.29 (s, 6H), 3.19 (bd, J=15.7 Hz, 2H), 3.01 (dd, J=15.0, 8.1 Hz, 2H). ¹³C NMR (126 MHz, DMSO-d₆) δ 187.6, 165.7, 154.9, 149.6 (dd, J_{CF}=250.7 Hz, J_{C-CF}=12.6 Hz, 2C), 149.3 (dd, J_{CF}=246.9 Hz, J_{C-CF}=12.6 Hz, 2C), 143.4, 135.1 (2C), 134.7 (2C), 132.7 (dd, J_{C-C-CF}=6.3 Hz, J_{C-C-CF}=3.7 Hz, 2C), 128.0 (2C), 127.6 (dd, J_{C-C-CF}=6.3 Hz, J_{C-C-CF}=3.7 Hz, 2C), 126.9, 119.1 (d, J_{C-CF}=17.6 Hz, 2C), 118.0 (2C), 117.8 (d, J_{C-CF}=17.6 Hz, 2C), 70.9, 58.2, 47.1, 45.0, 33.1. ¹⁹F NMR (376 MHz, DMSO-d₆) δ −136.9 (d, J_{FF}=22.5 Hz, 2F), −137.9 (d, J_{FF}=22.5 Hz, 2F). HR-APCI m/z calcd for C₃₄H₃₄F₄N₃O₅ [M+H]=640.2429, found 640.2407.

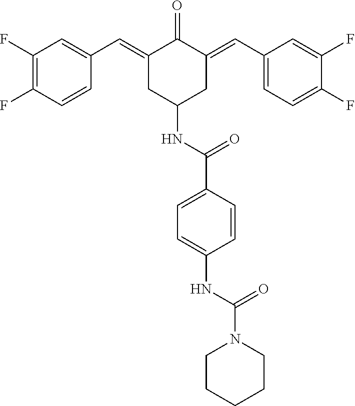 [SMILES: O=C1/C(=C/c2ccc(F)c(F)c2)CC(NC(=O)c2ccc(NC(=O)N3CCCCC3)cc2)C/C1=C\c1ccc(F)c(F)c1]

### N-(4-((3,5-bis((E)-3,4-difluorobenzylidene)-4-oxocyclohexyl)carbamoyl)phenyl)piperidine-1-carboxamide (JCS053)

¹H NMR (500 MHz, DMSO-d₆) δ 8.68 (s, 1H), 8.39 (d, J=6.5 Hz, 1H), 7.73 (d, J=8.5 Hz, 2H), 7.69-7.61 (m, 4H), 7.55-7.51 (m, 4H), 7.49-7.40 (m, 2H), 4.10-4.05 (m, 1H), 3.42 (t, J=5.4 Hz, 4H), 3.19 (bd, J=15.0 Hz, 2H), 3.01 (dd, J=15.0, 8.1 Hz, 2H), 1.59-1.55 (m, 2H), 1.50-1.46 (m, 4H). ¹³C NMR (126 MHz, DMSO-d₆) δ 187.6, 165.7, 154.4, 149.6 (dd, J_{CF}=250.7 Hz, J_{C-CF}=12.6 Hz, 2C), 149.3 (dd, J_{CF}=246.9 Hz, J_{C-CF}=12.6 Hz, 2C), 143.8, 135.1 (2C), 134.7 (2C), 132.7 (dd, J_{C-C-CF}=6.3 Hz, J_{C-C-CF}=3.7 Hz, 2C), 127.8 (2C), 127.6 (dd, J_{C-C-CF}=6.3 Hz, J_{C-C-CF}=3.7 Hz, 2C), 126.6, 119.1 (d, J_{C-CF}=17.6 Hz, 2C), 118.0 (2C), 117.8 (d, J_{C-CF}=17.6 Hz, 2C), 45.0, 44.6, 33.1, 25.5, 24.0. ¹⁹F NMR (376 MHz, DMSO-d₆) δ −136.9 (d, J_{FF}=22.5 Hz, 2F), −137.9 (d, J_{FF}=22.5 Hz, 2F). HR-APCI m/z calcd for C₃₃H₃₀F₄N₃O₃ [M+H]=592.2217, found 592.2202.

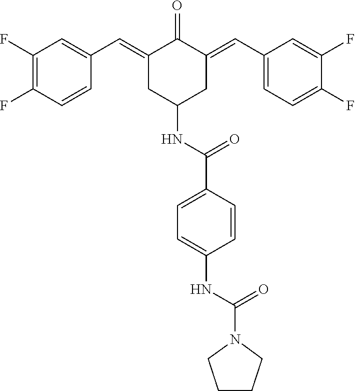 [SMILES: O=C1/C(=C/c2ccc(F)c(F)c2)CC(NC(=O)c2ccc(NC(=O)N3CCCC3)cc2)C/C1=C\c1ccc(F)c(F)c1]

### N-(4-((3,5-bis((E)-3,4-difluorobenzylidene)-4-oxocyclohexyl)carbamoyl)phenyl)pyrrolidine-1-carboxamide (JCS054)

¹H NMR (500 MHz, DMSO-d₆) δ 8.39 (d, J=6.6 Hz, 1H), 8.35 (s, 1H), 7.73 (d, J=8.5 Hz, 2H), 7.70-7.58 (m, 6H), 7.54-7.49 (m, 2H), 7.46-7.38 (m, 2H), 4.13-4.04 (m, 1H), 3.37 (t, J=5.0 Hz, 4H), 3.19 (bd, J=15.5 Hz, 2H), 3.01 (dd, J=15.0, 8.1 Hz, 2H), 1.86-1.84 (m, 4H). ¹³C NMR (126 MHz, DMSO-d₆) δ 187.5, 165.7, 153.5, 149.6 (dd, J_{CF}=250.7 Hz, J_{C-CF}=12.6 Hz, 2C), 149.3 (dd, J_{CF}=246.9 Hz, J_{C-CF}=12.6 Hz, 2C), 143.6, 135.1 (2C), 134.6 (2C), 132.7 (dd, J_{C-C-CF}=6.3 Hz, J_{C-C-CF}=3.7 Hz, 2C), 127.8 (2C), 127.6 (dd, J_{C-C-CF}=6.3 Hz, J_{C-C-CF}=3.7 Hz, 2C), 126.6, 119.1 (d, J_{C-CF}=17.6 Hz, 2C), 117.9 (2C), 117.8 (d, J_{C-CF}=17.6 Hz, 2C), 45.7, 45.0, 33.1, 25.0. ¹⁹F NMR (376 MHz, DMSO-d₆) δ −136.9 (d, J_{FF}=22.5 Hz, 2F), −137.9 (d, J_{FF}=22.5 Hz, 2F). HR-APCI m/z calcd for C₃₂H₂₈F₄N₃O₃ [M+H]=578.2061, found 578.2042.

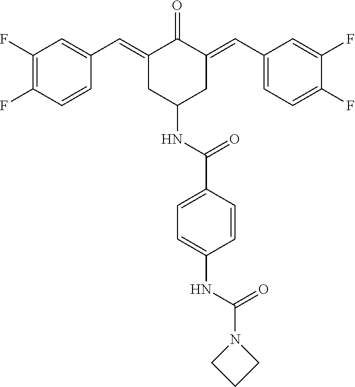 [SMILES: O=C1/C(=C/c2ccc(F)c(F)c2)CC(NC(=O)c2ccc(NC(=O)N3CCC3)cc2)C/C1=C\c1ccc(F)c(F)c1]

### N-(4-((3,5-bis((E)-3,4-difluorobenzylidene)-4-oxocyclohexyl)carbamoyl)phenyl)azetidine-1-carboxamide (JCS055)

¹H NMR (500 MHz, DMSO-d₆) δ 8.59 (s, 1H), 8.39 (d, J=6.5 Hz, 1H), 7.73 (d, J=8.4 Hz, 2H), 7.70-7.48 (m, 8H), 7.49-7.40 (m, 2H), 4.13-4.04 (m, 1H), 3.96 (t, J=7.6 Hz, 4H), 3.18 (bd, J=15.6 Hz, 2H), 3.01 (dd, J=15.0, 8.1 Hz, 2H), 2.20-2.14 (m, 4H). ¹³C NMR (126 MHz, DMSO-d₆) δ 187.5, 165.7, 156.2, 149.6 (dd, J_{CF}=250.7 Hz, J_{C-CF}=12.6 Hz, 2C), 149.3 (dd, J_{CF}=246.9 Hz, J_{C-CF}=12.6 Hz, 2C), 143.2, 135.1 (2C), 134.6 (2C), 132.7 (dd, J_{C-C-CF}=6.3 Hz, J_{C-C-CF}=3.7 Hz, 2C), 128.0 (2C), 127.6 (dd, J_{C-C-CF}=6.3 Hz, J_{C-C-CF}=3.7 Hz, 2C), 127.6, 119.1 (d, J_{C-CF}=17.6 Hz, 2C), 117.7 (d, J_{C-CF}=17.6 Hz, 2C), 117.3 (2C), 49.3, 45.0, 33.1, 14.7. ¹⁹F NMR (376 MHz, DMSO-d₆) δ 129.3 (d, J_{FF}=30.8 Hz, 2F), 128.3 (d, J_{FF}=30.8 Hz, 2F). HR-APCI m/z calcd for C₃₁H₂₆F₄N₃O₃ [M+H]=564.1904, found 564.1892.

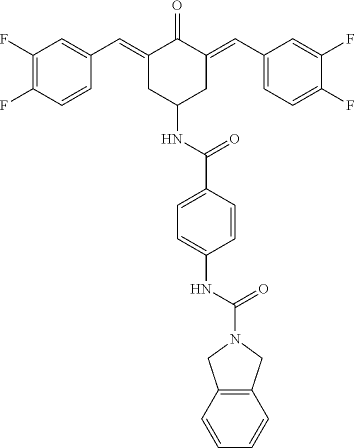 [SMILES: O=C1/C(=C/c2ccc(F)c(F)c2)CC(NC(=O)c2ccc(NC(=O)N3Cc4ccccc4C3)cc2)C/C1=C\c1ccc(F)c(F)c1]

### N-(4-((3,5-bis((E)-3,4-difluorobenzylidene)-4-oxocyclohexyl)carbamoyl)phenyl)isoindoline-2-carboxamide (JCS056)

¹H NMR (500 MHz, Acetone-d₆) δ 8.00 (s, 1H), 7.77 (d, J=5.0 Hz, 2H), 7.71-7.69 (m, 3H), 7.58-7.53 (m, 2H), 7.46-7.29 (m, 8H), 4.84 (s, 4H), 4.37-4.29 (m, 1H), 3.41 (bd, J=15.1 Hz, 2H), 3.15 (dd, J=15.2, 12.1 Hz, 2H). ¹³C NMR (126 MHz, Acetone-d₆) δ 188.1, 166.8, 154.4, 151.0 (dd, J_{CF}=250.7 Hz, J_{C-CF}=12.6 Hz, 2C), 150.8 (dd, J_{CF}=246.9 Hz, J_{C-CF}=12.6 Hz, 2C), 144.5, 137.8 (2C), 136.3, 135.6 (2C), 134.0 (dd, J_{C-C-CF}=6.3 Hz, J_{C-C-CF}=3.7 Hz, 2C), 128.7 (2C), 128.6, 128.3 (dd, J_{C-C-CF}=6.3 Hz, J_{C-C-CF}=3.7 Hz, 2C), 128.2, 123.6, 118.4 (d, J_{C-CF}=17.6 Hz, 2C), 118.9 (2C), 118.7 (d, J_{C-CF}=17.6 Hz, 2C), 52.7, 46.3, 34.4. ¹⁹F NMR (376 MHz, Acetone-d₆) δ −129.3 (d, J_{FF}=30.8 Hz, 2F), −128.3 (d, J_{FF}=30.8 Hz, 2F). HR-APCI m/z calcd for C₃₆H₂₈F₄N₃O₃ [M+H]=626.2061, found 626.2044.

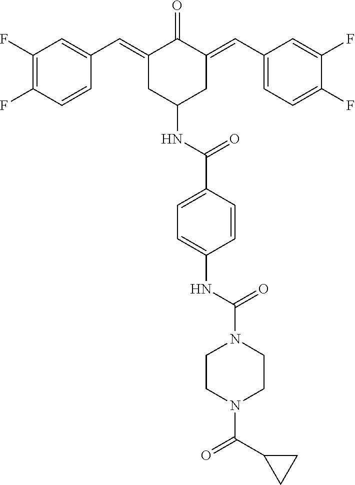 [SMILES: O=C1/C(=C/c2ccc(F)c(F)c2)CC(NC(=O)c2ccc(NC(=O)N3CCN(C(=O)C4CC4)CC3)cc2)C/C1=C\c1ccc(F)c(F)c1]

### N-(4-((3,5-bis((E)-3,4-difluorobenzylidene)-4-oxocyclohexyl)carbamoyl)phenyl)-4-(cyclopropanecarbonyl)piperazine-1-carboxamide (JCS057)

¹H NMR (500 MHz, CDCl₃) δ 7.70 (s, 2H), 7.54 (s, 1H), 7.52 (d, J=8.4 Hz, 2H), 7.33 (d, J=8.4 Hz, 2H), 7.22-7.11 (m, 6H), 6.89 (d, J=7.3 Hz, 1H), 4.40-4.33 (m, 1H), 3.65-3.40 (m, 8H), 3.23 (bd, J=15.2 Hz, 2H), 3.01 (dd, J=15.2, 9.9 Hz, 2H), 0.95-0.92 (m, 1H), 1.72-1.67 (m, 2H), 0.83-0.70 (m, 2H). ¹³C NMR (126 MHz, CDCl₃) δ 188.0, 172.6, 167.1, 154.9, 150.6 (dd, J_{CF}=253.2 Hz, J_{C-CF}=12.6 Hz, 2C), 150.2 (dd, J_{CF}=249.4 Hz, J_{C-CF}=12.6 Hz, 2C), 142.6, 137.5 (2C), 133.1 (2C), 132.2 (dd, J_{C-C-CF}=6.3 Hz, J_{C-C-CF}=3.7 Hz, 2C), 128.3 (2C), 127.9, 127.2 (dd, J_{C-C-CF}=6.3 Hz, J_{C-C-CF}=3.7 Hz, 2C), 119.5 (2C), 118.8 (d, J_{C-CF}=17.6 Hz, 2C), 117.7 (d, J_{C-CF}=17.6 Hz, 2C), 45.2, 45.1, 44.1, 43.5, 41.5, 33.82, 11.1, 7.9. ¹⁹F NMR (376 MHz, CDCl₃) δ 131.1 (d, J_{FF}=26.3 Hz, 2F), 129.6 (d, J_{FF}=26.3 Hz, 2F). HR-APCI m/z calcd for C₃₆H₃₃F₄N₄O₄ [M+H]=661.2432, found 661.2411.

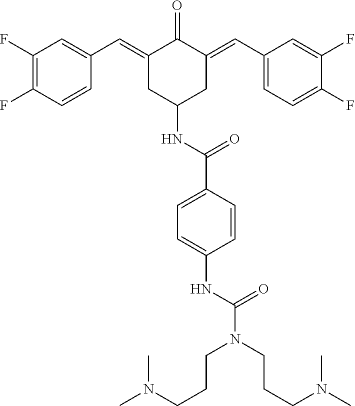 [SMILES: CN(C)CCCN(CCCN(C)C)C(=O)Nc1ccc(C(=O)NC2C/C(=C\c3ccc(F)c(F)c3)C(=O)/C(=C/c3ccc(F)c(F)c3)C2)cc1]

### N-(3,5-bis((E)-3,4-difluorobenzylidene)-4-oxocyclohexyl)-4-(3,3-bis(3-(dimethylamino)propyl)ureido)benzamide (JCS058)

¹H NMR (500 MHz, Acetone-d₆) δ 10.40 (s, 1H), 7.77 (d, J=8.5 Hz, 3H), 7.70 (s, 2H), 7.59-7.51 (m, 4H), 7.42 (dd, J=8.9, 6.6 Hz, 4H), 4.35-4.27 (m, 1H), 3.55 (t, J=6.2 Hz, 4H), 3.39 (bd, J=15.8 Hz, 2H), 3.15 (dd, J=15.2, 12.1 Hz, 2H), 2.97-2.88 (m, 4H), 2.80-2.67 (m, 12H). ¹³C NMR (126 MHz, Acetone-d₆) δ 188.1, 166.7, 158.9, 151.0 (dd, J_{CF}=250.7 Hz, J_{C-CF}=12.6 Hz, 2C), 150.8 (dd, J_{CF}=246.9 Hz, J_{C-CF}=12.6 Hz, 2C), 144.6, 136.3 (2C), 135.6 (2C), 134.0 (dd, J_{C-C-CF}=6.3 Hz, J_{C-C-CF}=3.7 Hz, 2C), 128.7 (2C), 128.9, 128.4 (dd, J_{C-C-CF}=6.3 Hz, J_{C-C-CF}=3.7 Hz, 2C), 119.7 (d, J_{C-CF}=17.6 Hz, 2C), 119.0 (2C), 118.4 (d, J_{C-CF}=17.6 Hz, 2C), 55.1, 46.3, 43.9, 42.7, 34.3, 23.9. ¹⁹F NMR (376 MHz, Acetone-d₆) δ −138.5 (d, J_{FF}=22.5 Hz, 2F),−139.3 (d, J_{FF}=18.8 Hz, 2F). HR-APCI m/z calcd for C₃₈H₄₄F₄N₅O₃ [M+H]=694.3374, found 694.3348.

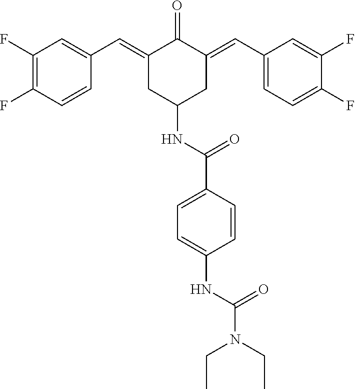 [SMILES: CCN(CC)C(=O)Nc1ccc(C(=O)NC2C/C(=C\c3ccc(F)c(F)c3)C(=O)/C(=C/c3ccc(F)c(F)c3)C2)cc1]

### N-(3,5-bis((E)-3,4-difluorobenzylidene)-4-oxocyclohexyl)-4-(3,3-diethylureido)benzamide (JCS059)

¹H NMR (500 MHz, CDCl₃) δ 7.79 (s, 2H), 7.59 (dd, J=8.7, 3.2 Hz, 2H), 7.42 (dd, J=8.7, 3.1 Hz, 2H), 7.27-7.16 (m, 6H), 6.47 (s, 1H), 6.28-6.19 (m, 1H), 4.50-4.43 (m, 1H), 3.40-3.34 (m, 4H), 3.26 (bd, J=15.7 Hz, 2H), 3.04 (dd, J=15.8, 8.3 Hz, 2H), 1.22 (t, J=7.1 Hz, 6H). ¹³C NMR (126 MHz, CDCl₃) δ 188.0, 166.7, 154.1, 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 142.8, 137.8 (2C), 132.9 (2C), 132.2 (dd, J_{C-C-CF}=6.3 Hz, J_{C-C-CF}=3.7 Hz, 2C), 128.0 (2C), 127.8, 127.1 (dd, J_{C-C-CF}=6.3 Hz, J_{C-C-CF}=3.7 Hz, 2C), 119.0 (d, J_{C-CF}=17.6 Hz, 2C), 118.9 (2C), 117.7 (d, J_{C-CF}=17.6 Hz, 2C), 44.8, 41.8, 33.9, 14.0. ¹⁹F NMR (376 MHz, CDCl₃) δ −135.1 (d, J_{FF}=22.5 Hz, 2F), −136.5 (d, J_{FF}=22.5 Hz, 2F). HR-APCI m/z calcd for C₃₂H₃₀F₄N₃O₃ [M+H]=580.2217, found 580.2198.

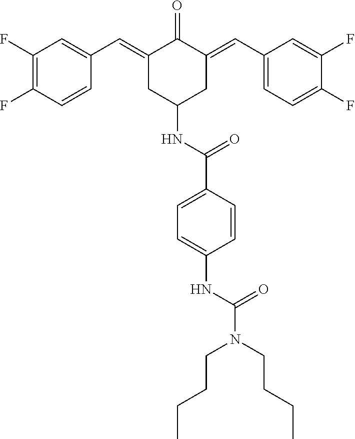 [SMILES: CCCCN(CCCC)C(=O)Nc1ccc(C(=O)NC2C/C(=C\c3ccc(F)c(F)c3)C(=O)/C(=C/c3ccc(F)c(F)c3)C2)cc1]

### N-(3,5-bis((E)-3,4-difluorobenzylidene)-4-oxocyclohexyl)-4-(3,3-dibutylureido)benzamide (JCS060)

¹H NMR (500 MHz, CDCl₃) δ 7.78 (s, 2H), 7.59 (d, J=8.3 Hz, 2H), 7.40 (d, J=8.3 Hz, 2H), 7.26-7.14 (m, 6H), 6.49 (s, 1H), 6.31 (d, J=7.3 Hz, 1H), 4.50-4.42 (m, 1H), 3.30-3.23 (m, 6H), 3.03 (dd, J=15.5, 8.6 Hz, 2H), 1.62-1.55 (m, 4H), 1.40-1.31 (m, 4H), 0.95 (t, J=7.3 Hz, 6H). ¹³C NMR (126 MHz, CDCl₃) δ 188.0, 166.7, 154.4, 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 142.8, 137.8 (2C), 133.0 (2C), 132.2 (dd, J_{C-C-CF}=6.3 Hz, J_{C-C-CF}=3.7 Hz, 2C), 128.0 (2C), 127.7, 127.1 (dd, J_{C-C-CF}=6.3 Hz, J_{C-C-CF}=3.7 Hz, 2C), 119.0 (d, J_{C-CF}=17.6 Hz, 2C), 118.8 (2C), 117.7 (d, J_{C-CF}=17.6 Hz, 2C), 47.6, 44.8, 33.9, 30.8, 20.3, 14.0. ¹⁹F NMR (376 MHz, CDCl₃) δ −135.1 (d, J_{FF}=22.5 Hz, 2F), −136.5 (d, J_{FF}=22.5 Hz, 2F). HR-APCI m/z calcd for C₃₆H₃₈F₄N₃O₃ [M+H]=636.2843, found 636.2805.

### General Procedure C:

The compounds were generally prepared by reaction of the corresponding aldehydes, e.g., 3,4-difluorobenzaldehyde, with tert-butyl (4-oxocyclohexyl)carbamate in the present of 20% aq. sodium hydroxide to give N-tert-butyl (3,5-bis((E)-3,4-difluorobenzylidene)-4-oxocyclohexyl)carbamate. Treatment of the product with trifluoroacetic acid (TFA) caused deprotection of the Boc group. The BOC deprotected amine was subjected to hydrogenation to obtain 4-amino-2,6-bis(3,4-difluorobenzyl)cyclohexan-1-one. Benzoylation of 4-amino-2,6-bis(3,4-difluorobenzyl)cyclohexan-1-one with 4-((2-(pyrrolidin-1-yl)ethyl)amino)benzoic acid using standard peptide coupling reagents TBTU or EDC and HOAt afforded the N-(3,5-bis(3,4-difluorobenzyl)-4-oxocyclohexyl)-4-((2-(pyrrolidin-1-yl)ethyl)amino)benzamide.

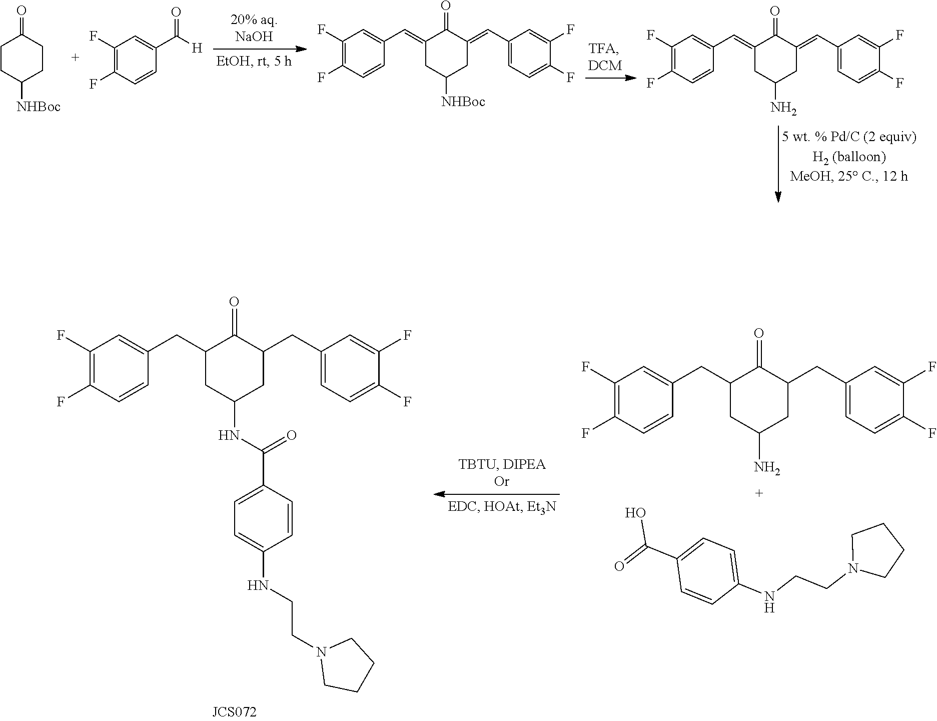

### Example JCS072

To the mixture of the N-tert-butyl (4-oxocyclohexyl)carbamate (213.28 mg, 1 mmol, 1.0 equiv.) and ethanol (1.0 mL) in a round bottom flask added dropwise 20% aq. sodium hydroxide (1.5 mL) and stirred for five minutes. To this mixture was added 3,4-difluorobenzaldehyde (355.3 mg, 2.5 mmol, 2.5 equiv.). The reaction mixture was then allowed to stir at 21° C. for 5 h. After 5 h the yellow precipitate thus obtained was filtered, washed with water, cold ethanol and dried to get the pure product (360 mg, 78% yield).

Trifluoroacetic acid (1.0 ml) was added to a solution of N-tert-butyl (3,5-bis((E)-3,4-difluorobenzylidene)-4-oxocyclohexyl)carbamate (461.4 mg, 1.0 mmol) in dichloromethane (5.0 ml) at 21° C. and stirred overnight at 21° C. Then, the solvent of the reaction solution was distilled off under reduced pressure and the resulting residue was poured into a 1N aqueous sodium hydroxide solution and extracted with ethyl acetate. The organic layer was washed with a saturated aqueous sodium chloride solution and then dried over anhydrous magnesium sulfate. The solvent was distilled off under reduced pressure to obtain 4-amino-2,6-bis((E)-3,4-difluorobenzylidene)cyclohexan-1-one.

The mixture of 4-amino-2,6-bis((E)-3,4-difluorobenzylidene)cyclohexan-1-one (361.3 mg, 1.0 mmol, 1.0 equiv.) and 5 wt. % Pd/C (212.8 mg, 2.0 mmol, 2.0 equiv.) in MeOH were stirred in the atmosphere of hydrogen at 21° C. for 24 h. Then, the resulting solution was filtered through a pad of Celite, concentrated to give 4-amino-2,6-bis(3,4-difluorobenzyl)cyclohexan-1-one which was used for next step without further purification.

The mixture of 4-amino-2,6-bis(3,4-difluorobenzyl)cyclohexan-1-one (182.6 mg, 0.5 mmol, 1.0 equiv.) and anhydrous diisopropylethylamine (261.2 μL, 1.5 mmol, 3.0 equiv.) in THE was maintained at 0° C. (ice bath). To this cooled mixture was added dropwise 4-((2-(pyrrolidin-1-yl)ethyl)amino)benzoic acid (117.0 mg, 0.5 mmol, 1.0 equiv.) in 2.0 mL THF followed by and TBTU (240.8 mg, 0.75 mmol, 1.5 equiv.). After the complete addition of 4-((2-(pyrrolidin-1-yl)ethyl)amino)-benzoic acid, the reaction mixture was slowly warmed up to room temperature and stirred overnight. After completion of the reaction, the solvent was evaporated and the residue was stirred in sat. aq, NaHCO₃ for 5 min. The mixture was extracted with ethyl acetate three times. The organic layer was washed with a saturated aqueous sodium chloride solution and then dried over anhydrous sodium sulfate. The solvent was evaporated, followed by flash chromatography (gradient elution 50% methanol/ethyl acetate-100% methanol) to give desired product JCS072 (87 mg, 30% yield) as white solid.

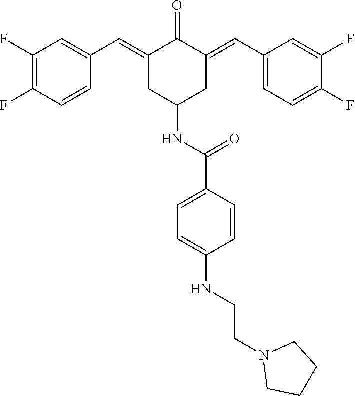 [SMILES: O=C1/C(=C/c2ccc(F)c(F)c2)CC(NC(=O)c2ccc(NCCN3CCCC3)cc2)C/C1=C\c1ccc(F)c(F)c1]

### N-(3,5-bis(3,4-difluorobenzyl)-4-oxocyclohexyl)-4-((2-(pyrrolidin-1-yl)ethyl)amino)benzamide (JCS072)

¹H NMR (500 MHz, CDCl₃) δ 7.55 (d, J=8.4 Hz, 2H), 7.09-6.78 (m, 6H), 6.54 (d, J=8.4 Hz, 2H), 5.85 (d, J=8.2 Hz, 1H), 4.76 (s, 1H), 4.50-4.42 (m, 1H), 3.20 (t, J=6.3 Hz, 2H), 3.10 (bd, J=15.0 Hz, 2H), 2.73 (t, J=6.2 Hz, 4H), 2.56-2.52 (m, 4H), 2.43 (dd, J=15.3, 7.4 Hz, 2H), 2.28 (t, J=6.3 Hz, 2H), 1.80-1.76 (m, 4H), 1.35 (q, J=12.5 Hz, 2H). ¹³C NMR (126 MHz, CDCl₃) δ 210.3, 166.7, 151.4, 150.2 (dd, J_{CF}=249.4 Hz, J_{C-CF}=12.6 Hz, 2C), 149.1 (dd, J_{CF}=246.9 Hz, J_{C-CF}=12.6 Hz, 2C), 136.5 (dd, J_{C-C-CF}=6.3 Hz, J_{C-C-CF}=3.7 Hz, 2C), 128.7 (2C), 125.0 (dd, J_{C-C-CF}=6.3 Hz, J_{C-C-CF}=3.7 Hz, 2C), 121.7, 118.1 (d, J_{C-CF}=17.6 Hz, 2C), 117.1 (d, J_{C-CF}=17.6 Hz, 2C), 111.8 (2C), 54.5, 53.9, 49.8, 46.8, 41.8, 39.8, 34.4, 23.5. ¹⁹F NMR (376 MHz, CDCl₃) δ −137.9 (d, J_{FF}=22.5 Hz, 2F), −141.5 (d, J_{FF}=18.8 Hz, 2F). HR-APCI m/z calcd for C₃₃H₃₆F₄N₃O₂ [M+H]=582.2738, found 582.2738.

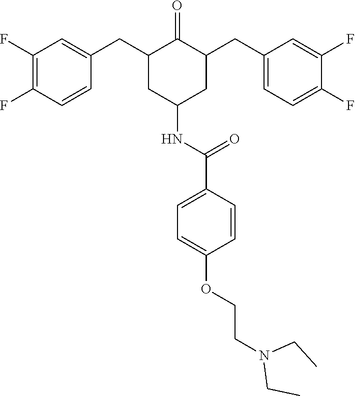 [SMILES: CCN(CC)CCOc1ccc(C(=O)NC2CC(Cc3ccc(F)c(F)c3)C(=O)C(Cc3ccc(F)c(F)c3)C2)cc1]

### N-(3,5-bis(3,4-difluorobenzyl)-4-oxocyclohexyl)-4-(2-(diethylamino)ethoxy)benzamide (JCS074)

¹H NMR (500 MHz, CDCl₃) δ 7.64 (d, J=8.3 Hz, 2H), 7.09-6.92 (m, 4H), 6.91-6.82 (m, 4H), 5.92 (d, J=8.0 Hz, 1H), 4.51-4.42 (m, 1H), 4.12 (t, J=5.9 Hz, 2H), 3.12 (dd, J=14.1, 5.8 Hz, 2H), 2.95 (t, J=5.9 Hz, 2H), 2.77-2.68 (m, 4H), 2.45 (dd, J=14.2, 7.4 Hz, 2H), 2.32-2.29 (m, 2H), 1.38 (q, J=12.6 Hz, 2H), 1.10 (t, J=7.1 Hz, 6H). ¹³C NMR (126 MHz, CDCl₃) δ 210.1, 166.3, 161.5, 150.2 (dd, J_{CF}=248.2 Hz, J_{C-CF}=12.6 Hz, 2C), 149.1 (dd, J_{CF}=246.9 Hz, J_{C-CF}=12.6 Hz, 2C), 136.5 (dd, J_{C-C-CF}=6.3 Hz, J_{C-C-CF}=3.7 Hz, 2C), 128.8 (2C), 126.4 (2C), 125.0 (dd, J_{C-C-CF}=6.3 Hz, J_{C-C-CF}=3.7 Hz, 2C), 121.7, 118.0 (d, J_{C-CF}=17.6 Hz, 2C), 117.2 (d, J_{C-CF}=17.6 Hz, 2C), 114.4 (2C), 66.4, 51.5, 49.8, 47.8, 47.0, 39.6, 34.4, 11.4. ¹⁹F NMR (376 MHz, CDCl₃) δ 131.1 (d, J_{FF}=26.3 Hz, 2F), 128.3 (d, J_{FF}=30.0 Hz, 2F). HR-APCI m/z calcd for C₃₃H₃₇F₄N₃O₂ [M+H]=585.2734, found 585.2725.

### General Procedure D:

Compounds JCS007 and their analogues were generally prepared by reaction of 3,4-difluoro-benzaldehyde, with tert-butyl (4-oxocyclohexyl)carbamate in the present of 20% aq. sodium hydroxide to give tert-butyl (3,5-bis((E)-3,4-difluorobenzylidene)-4-oxocyclohexyl)carbamate. The Boc protecting group was deprotected with TFA. Acylation of the tert-butyl (3,5-bis((E)-3,4-difluorobenzylidene)-4-oxocyclohexyl)carbamate with acryloyl chloride under basic conditions afforded the N-(3,5-bis((E)-3,4-difluorobenzylidene)-4-oxocyclohexyl)acrylamide. Michael addition of an amine, e.g., pyrrolidine, afforded the N-(3,5-bis((E)-3,4-difluorobenzylidene)-4-oxocyclohexyl)-3-(pyrrolidin-1-yl)propenamide.

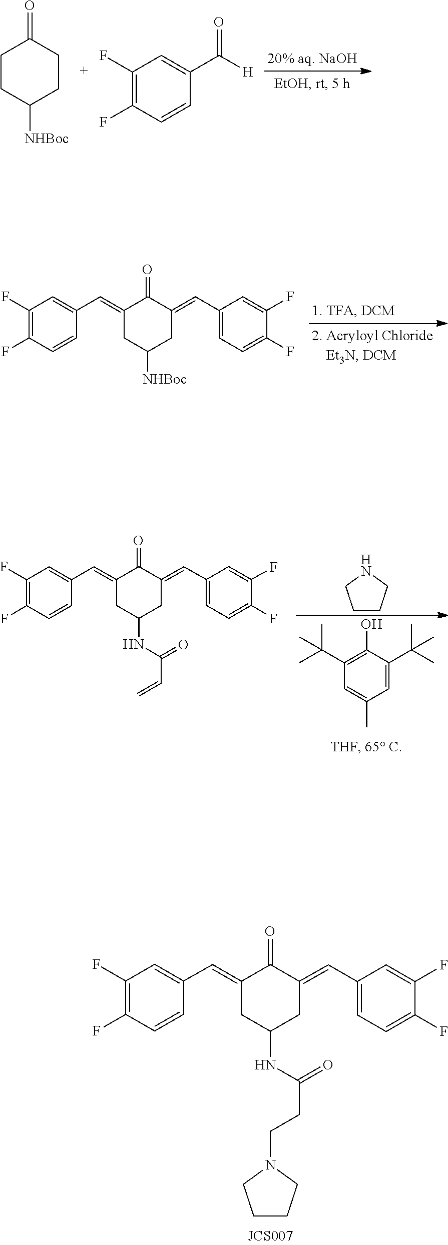

### Preparation of JCS007:

To a mixture of tert-butyl (4-oxocyclohexyl)carbamate (213.28 mg, 1 mmol, 1.0 equiv.) and ethanol (1.0 mL) in a round bottom flask was added dropwise 20% aqueous sodium hydroxide (1.0 mL) and the reaction mixture was stirred for 5 min. To this mixture was added 3,4-difluorobenzaldehyde (355.3 mg, 2.5 mmol, 2.5 equiv.). The reaction mixture was then allowed to stir at 21° C. for 5 h, at which time a yellow solid had precipitated. The precipitate thus obtained was filtered, washed with water and cold ethanol and dried to get the pure bis(arylmethylidene)cyclohexanone product (360 mg, 78% yield).

Trifluoroacetic acid (0.5 ml) was added to a solution of tert-butyl (3,5-bis((E)-3,4-difluorobenzylidene)-4-oxocyclohexyl)carbamate (230.7 mg, 0.5 mmol) in methylene chloride (5.0 ml) at 21° C. and the reaction mixture was stirred overnight at 21° C. The reaction solvent was distilled off under reduced pressure and the resulting residue was poured into a IN aqueous sodium hydroxide solution and extracted with ethyl acetate and chloroform. The organic layer was washed with a saturated aqueous sodium chloride solution and then dried over anhydrous magnesium sulfate. The solvent was distilled off under reduced pressure to obtain 4-amino-2,6-bis((E)-3,4-difluorobenzylidene)cyclohexan-1-one.

A mixture of 4-amino-2,6-bis((E)-3,4-difluorobenzylidene)cyclohexan-1-one (180.7 mg, 0.5 mmol, 1.0 equiv.) and anhydrous triethylamine (70 μL, 0.5 mmol, 1.0 equiv.) in dichloromethane was maintained at 0° C. (ice bath). To this cooled mixture was added dropwise acryloyl chloride (40 μL, 0.5 mmol, 1.0 equiv.). After the complete addition of the acryloyl chloride, the reaction mixture was slowly warmed up to 21° C. and stirred overnight. After completion of the reaction, the solvent was evaporated and the residue thus obtained was washed with water, filtered, and dried. The crude amide product was pure enough to be used for the next step.

A mixture of the crude N-(3,5-bis((E)-3,4-difluorobenzylidene)-4-oxocyclohexyl)acrylamide (207.7 mg, 0.5 mmol, 1.0 equiv.), 2,6-bis(1,1-dimethylethyl)-4-methylphenol (1.1 mg, 0.005 mmol, 1%) and pyrrolidine (0.375 mL, 0.75 mmol, 1.5 equiv.) in 1.0 mL anhydrous THF was heated to 65° C. under argon for 12 h. The solvent was evaporated and flash chromatography of the residue (gradient elution 20% methanol/EtOAC-100% methanol) gave the desired compound JCS007 (170.2 mg, 70% yield) as a yellow solid.

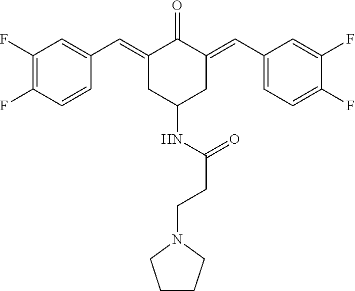 [SMILES: O=C(CCN1CCCC1)NC1C/C(=C\c2ccc(F)c(F)c2)C(=O)/C(=C/c2ccc(F)c(F)c2)C1]

### N-(3,5-bis((E)-3,4-difluorobenzylidene)-4-oxocyclohexyl)-3-(pyrrolidin-1-yl)propanamide (JCS007)

¹H NMR (500 MHz, CDCl₃) δ 9.32 (d, J=6.4 Hz, 1H), 7.80 (s, 2H), 7.26-7.16 (m, 6H), 4.50-4.38 (m, 1H), 3.13 (bd, J=15.9 Hz, 2H), 3.02 (dd, J=16.0, 5.7 Hz, 2H), 2.59 (t, J=5.3 Hz, 2H), 2.44-2.41 (m, 4H), 2.27 (t, J=5.3 Hz, 2H), 1.72-1.69 (m, 4H).

¹³C NMR (126 MHz, CDCl₃) δ 187.9, 172.6, 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 137.7 (2C), 132.8 (2C), 132.3 (dd, J_{C-C-CF}=6.3 Hz, J_{C-C-CF}=3.7 Hz, 2C), 127.1 (dd, J_{C-C-CF}=6.3 Hz, J_{C-C-CF}=3.7 Hz, 2C), 118.9 (d, J_{C-CF}=17.6 Hz, 2C), (2C), 117.7 (d, J_{C-CF}=17.6 Hz, 2C), 53.2, 51.5, 42.7, 33.7, 33.3, 23.6.

¹⁹F NMR (376 MHz, CDCl₃) δ −135.1 (d, J_{FF}=22.5 Hz, 2F), −136.5 (d, J_{FF}=22.5 Hz, 2F).

HR-APCI m/z calcd for C₂₇H₂₇F₄N₂O₂ [M+H]=487.2003, found 487.2018.

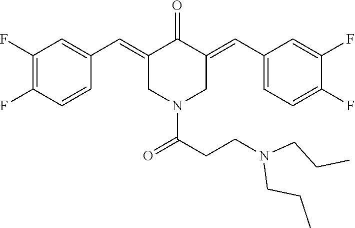 [SMILES: CCCN(CCC)CCC(=O)N1C/C(=C\c2ccc(F)c(F)c2)C(=O)/C(=C/c2ccc(F)c(F)c2)C1]

### 3,5-Bis((E)-3,4-difluorobenzylidene)-1-(3-(dipropylamino)propanoyl)piperidin-4-one (JCS117)

¹H NMR (500 MHz, CDCl₃) δ 7.71 (br s, 2H), 7.31-7.08 (m, 6H), 4.82 (s, 2H), 4.70 (s, 2H), 2.64 (t, J=7.4 Hz, 2H), 2.31 (t, J=7.4 Hz, 2H), 2.24-2.13 (m, 4H), 1.34-1.22 (m, 4H), 0.73 (t, J=7.4 Hz, 6H).

¹³C NMR (126 MHz, CDCl₃) δ 185.9, 171.0, 151.0 (dd, J_{CF}=254.5 Hz, J_{C-CF}=12.6 Hz, 2C), 150.4 (dd, J_{CF}=250.7 Hz, J_{C-CF}=12.6 Hz, 2C), 136.2, 135.2, 132.3, 132.2, 131.4 (d, J_{C-CF}=22.6 Hz, 2C), 127.1 (d, J_{C-CF}=45.3 Hz, 2C), 119.1 (dd, J_{C-C-CF}=39.0 Hz, J_{C-C-CF}=17.6 Hz, 2C), 117.9 (dd, J_{C-C-CF}=30.2 Hz, J_{C-C-CF}=17.6 Hz, 2C), 56.1, 49.8, 46.4, 43.1, 31.2, 20.1, 11.7. Due to slow amide rotation, one observes two absorptions for some carbons.

¹⁹F NMR (376 MHz, CDCl₃) δ −132.0 (dd, J_{FF}=154.1, 30.0 Hz, 2F), −130.3 (dd, J_{FF}=244.4, 26.3 Hz, 2F).

HR-APCI m/z calcd for C₂₈H₃₁F₄N₂O₂ [M+H]=503.2339, found 503.2316.

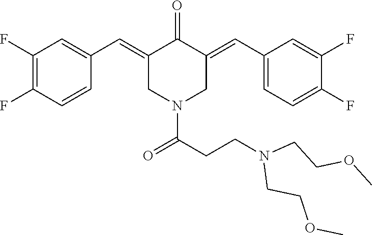 [SMILES: COCCN(CCOC)CCC(=O)N1C/C(=C\c2ccc(F)c(F)c2)C(=O)/C(=C/c2ccc(F)c(F)c2)C1]

### 1-(3-(Bis(2-methoxyethyl)amino)propanoyl)-3,5-bis((E)-3,4-difluorobenzylidene)piperidin-4-one (JCS118)

¹H NMR (500 MHz, CDCl₃) δ 7.70 (br s, 2H), 7.29-7.11 (m, 6H), 4.82 (s, 2H), 4.70 (s, 2H), 3.33 (t, J=5.7 Hz, 4H), 3.23 (s, 6H), 2.79 (t, J=5.8 Hz, 2H), 2.55 (t, J=5.8 Hz, 4H), 2.36 (t, J=5.8 Hz, 2H).

¹³C NMR (126 MHz, CDCl₃) δ 185.9, 170.7, 151.0 (dd, J_{CF}=254.5 Hz, J_{C-CF}=12.6 Hz, 2C), 150.4 (dd, J_{CF}=250.7 Hz, J_{C-CF}=12.6 Hz, 2C), 136.1, 135.2, 132.1 (2C), 131.4 (d, J_{C-CF}=34.0 Hz, 2C), 127.1 (d, J_{C-CF}=45.3 Hz, 2C), 119.1 (dd, J_{C-C-CF}=39.0 Hz, J_{C-C-CF}=17.6 Hz, 2C), 117.9 (dd, J_{C-C-CF}=30.2 Hz, J_{C-C-CF}=17.6 Hz, 2C), 70.7, 58.7, 53.8, 50.7, 46.3, 42.9, 30.8. Due to slow amide rotation, one observes two absorptions for some carbons.

¹⁹F NMR (376 MHz, CDCl₃) δ −134.2 (dd, J_{FF}=154.1, 30.0 Hz, 2F), −135.9 (dd, J_{FF}=244.4, 26.3 Hz, 2F).

HR-APCI m/z calcd for C₂₈H₃₁F₄N₂O₂ [M+H]=535.2334, found 535.2214.

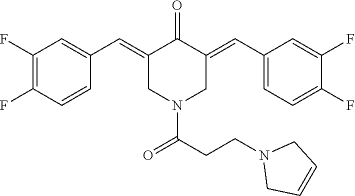 [SMILES: O=C1/C(=C/c2ccc(F)c(F)c2)CN(C(=O)CCN2CC=CC2)C/C1=C\c1ccc(F)c(F)c1]

### 3,5-Bis((E)-3,4-difluorobenzylidene)-1-(3-(2,5-dihydro-1H-pyrrol-1-yl)propanoyl)piperidin-4-one (JCS119)

¹H NMR (500 MHz, CDCl₃) δ 7.73 (br s, 2H), 7.26-7.13 (m, 6H), 5.69 (s, 2H), 4.85 (s, 2H), 4.72 (s, 2H), 3.33 (s, 4H), 2.85 (t, J=5.8 Hz, 2H), 2.39 (t, J=5.8 Hz, 2H).

¹³C NMR (126 MHz, CDCl₃) δ 185.8, 170.2, 151.0 (dd, J_{CF}=254.5 Hz, J_{C-CF}=12.6 Hz, 2C), 150.4 (dd, J_{CF}=250.7 Hz, J_{C-CF}=12.6 Hz, 2C), 136.3, 135.3, 132.1 (2C), 131.4 (d, J_{C-CF}=34.0 Hz, 2C), 127.2 (2C), 127.1 (d, J_{C-CF}=45.3 Hz, 2C), 119.1 (dd, J_{C-C-CF}=39.0 Hz, J_{C-C-CF}=17.6 Hz, 2C), 117.9 (dd, J_{C-C-CF}=30.2 Hz, J_{C-C-CF}=17.6 Hz, 2C), 59.7, 51.4, 46.2, 43.0, 32.7. Due to slow amide rotation, one observes two absorptions for some carbons.

¹⁹F NMR (376 MHz, CDCl₃) δ −134.0 (dd, J_{FF}=154.1, 30.0 Hz, 2F), −135.8 (dd, J_{FF}=244.4, 26.3 Hz, 2F).

HR-APCI m/z calcd for C₂₆H₂₃F₄N₂O₂ [M+H]=471.1712, found 471.1690.

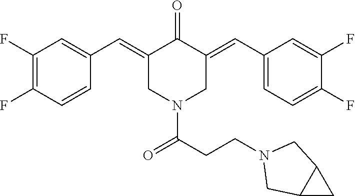 [SMILES: O=C1/C(=C/c2ccc(F)c(F)c2)CN(C(=O)CCN2CC3CC3C2)C/C1=C\c1ccc(F)c(F)c1]

### 1-(3-(3-Azabicyclo[3.1.0]hexan-3-yl)propanoyl)-3,5-bis((E)-3,4-difluorobenzylidene)piperidin-4-one (JCS120)

¹H NMR (500 MHz, CDCl₃) δ 7.76 (br s, 2H), 7.44-7.06 (m, 6H), 4.87 (s, 2H), 4.74 (s, 2H), 2.86 (d, J=8.6 Hz, 2H), 2.70 (t, J=5.8 Hz, 2H), 2.39 (t, J=5.8 Hz, 2H), 2.28 (d, J=8.7 Hz, 2H), 1.36-1.31 (m, 2H), 0.62-0.58 (m, 1H), 0.41-0.34 (m, 1H).

¹³C NMR (126 MHz, CDCl₃) δ 185.9, 170.7, 151.0 (dd, J_{CF}=254.5 Hz, J_{C-CF}=12.6 Hz, 2C), 150.4 (dd, J_{CF}=250.7 Hz, J_{C-CF}=12.6 Hz, 2C), 136.2, 135.3, 132.1 (2C), 131.4 (d, J_{C-CF}=34.0 Hz, 2C), 127.1 (d, J_{C-CF}=45.3 Hz, 2C), 119.1 (dd, J_{C-C-CF}=39.0 Hz, J_{C-C-CF}=17.6 Hz, 2C), 118.0 (dd, J_{C-C-CF}=30.2 Hz, J_{C-C-CF}=17.6 Hz, 2C), 55.0, 50.8, 46.4, 43.2, 32.4, 15.2, 6.9. Due to slow amide rotation, one observes two absorptions for some carbons.

¹⁹F NMR (376 MHz, CDCl₃) δ −132.0 (dd, J_{FF}=154.1, 30.0 Hz, 2F), −130.4 (dd, J_{FF}=244.4, 26.3 Hz, 2F).

HR-APCI m/z calcd for C₂₇H₂₅F₄N₂O₂ [M+H]=485.1866, found 485.1846.

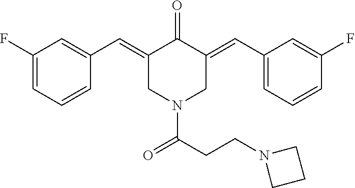 [SMILES: O=C1/C(=C/c2cccc(F)c2)CN(C(=O)CCN2CCC2)C/C1=C\c1cccc(F)c1]

### 1-(3-(Azetidin-1-yl)propanoyl)-3,5-bis((E)-3,4-difluorobenzylidene)piperidin-4-one (JCS121)

¹H NMR (500 MHz, CDCl₃) δ 7.74 (br s, 2H), 7.31-7.13 (m, 6H), 4.84 (s, 2H), 4.69 (s, 2H), 3.08 (t, J=7.1 Hz, 4H), 2.61 (t, J=7.4 Hz, 2H), 2.22 (t, J=7.4 Hz, 2H), 2.01-1.97 (m, 2H).

¹³C NMR (126 MHz, CDCl₃) δ 186.0, 170.2, 151.0 (dd, J_{CF}=254.5 Hz, J_{C-CF}=12.6 Hz, 2C), 150.4 (dd, J_{CF}=250.7 Hz, J_{C-CF}=12.6 Hz, 2C), 136.4, 135.5, 132.2 (2C), 131.5 (d, J_{C-CF}=34.0 Hz, 2C), 127.1 (d, J_{C-CF}=45.3 Hz, 2C), 119.1 (dd, J_{C-C-CF}=39.0 Hz, J_{C-C-CF}=17.6 Hz, 2C), 118.1 (dd, J_{C-C-CF}=30.2 Hz, J_{C-C-CF}=17.6 Hz, 2C), 55.1, 54.8, 46.4, 43.1, 31.2, 17.5. Due to slow amide rotation, one observes two absorptions for some carbons.

¹⁹F NMR (376 MHz, CDCl₃) δ −134.0 (dd, J_{FF}=154.1, 30.0 Hz, 2F), −135.8 (dd, J_{FF}=244.4, 26.3 Hz, 2F).

HR-APCI m/z calcd for C₂₅H₂₃F₄N₂O₂ [M+H]=459.1711, found 459.1690.

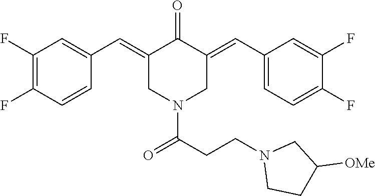 [SMILES: COC1CCN(CCC(=O)N2C/C(=C\c3ccc(F)c(F)c3)C(=O)/C(=C/c3ccc(F)c(F)c3)C2)C1]

### 3,5-Bis((E)-3,4-difluorobenzylidene)-1-(3-(3-methoxypyrrolidin-1-yl)propanoyl)piperidin-4-one (JCS122)

¹H NMR (500 MHz, CDCl₃) δ 7.73 (br s, 2H), 7.26-7.15 (m, 6H), 4.84 (s, 2H), 4.72 (s, 2H), 3.88-3.84 (m, 1H), 3.24 (s, 3H), 2.81 (t, J=7.5 Hz, 2H), 2.73-2.47 (m, 6H), 2.07-1.95 (m, 1H), 1.85-1.77 (m, 1H).

¹³C NMR (126 MHz, CDCl₃) δ 186.0, 170.0, 151.0 (dd, J_{CF}=254.5 Hz, J_{C-CF}=12.6 Hz, 2C), 150.4 (dd, J_{CF}=250.7 Hz, J_{C-CF}=12.6 Hz, 2C), 136.4, 135.7, 132.1 (2C), 131.5 (d, J_{C-CF}=34.0 Hz, 2C), 127.1 (d, J_{C-CF}=45.3 Hz, 2C), 119.1 (dd, J_{C-C-CF}=39.0 Hz, J_{C-C-CF}=17.6 Hz, 2C), 118.1 (dd, J_{C-C-CF}=30.2 Hz, J_{C-C-CF}=17.6 Hz, 2C), 79.9, 59.8, 56.6, 52.8, 51.6, 46.4, 43.1, 31.7, 31.1. Due to slow amide rotation, one observes two absorptions for some carbons.

¹⁹F NMR (376 MHz, CDCl₃) δ −134.0 (dd, J_{FF}=154.1, 30.0 Hz, 2F), −135.8 (dd, J_{FF}=244.4, 26.3 Hz, 2F).

HR-APCI m/z calcd for C₂₇H₂₇F₄N₂O₃ [M+H]=503.1971, found 503.1952.

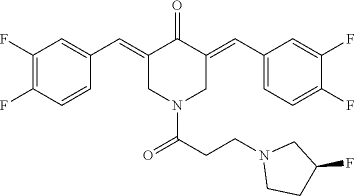 [SMILES: O=C1/C(=C/c2ccc(F)c(F)c2)CN(C(=O)CCN2CC[C@H](F)C2)C/C1=C\c1ccc(F)c(F)c1]

### 3,5-Bis((E)-3,4-difluorobenzylidene)-1-(3-((S)-3-fluoropyrrolidin-1-yl)propanoyl)piperidin-4-one (JCS123)

¹H NMR (500 MHz, CDCl₃) δ 7.72 (br s, 2H), 7.29-7.12 (m, 6H), 5.08 (dt, J=55.3, 5.7 Hz, 1H), 4.84 (br s, 2H), 4.70 (br s, 2H), 2.82-2.67 (m, 4H), 2.29-2.25 (m, 1H), 2.40 (t, J=7.5 Hz, 2H), 1.85-1.77 (m, 1H), 2.13-1.90 (m, 2H).

¹³C NMR (126 MHz, CDCl₃) δ 185.9, 170.3, 151.0 (dd, J_{CF}=254.5 Hz, J_{C-CF}=12.6 Hz, 2C), 150.4 (dd, J_{CF}=250.7 Hz, J_{C-CF}=12.6 Hz, 2C), 136.4, 135.4, 132.1 (2C), 131.4 (d, J_{C-CF}=34.0 Hz, 2C), 127.1 (d, J_{C-CF}=45.3 Hz, 2C), 119.1 (dd, J_{C-C-CF}=39.0 Hz, J_{C-C-CF}=17.6 Hz, 2C), 118.1 (dd, J_{C-C-CF}=30.2 Hz, J_{C-C-CF}=17.6 Hz, 2C), 93.3 (d, J_{CF}=176.4 Hz, 1C), 60.7 (d, J_{C-CF}=22.6 Hz, 2C), 56.6, 52.3, 51.2, 46.3, 43.2, 32.7 (d, J_{C-CF}=22.6 Hz, 2C), 32.2. Due to slow amide rotation, one observes two absorptions for some carbons.

¹⁹F NMR (376 MHz, CDCl₃) δ −134.0 (dd, J_{FF}=154.1, 30.0 Hz, 2F), −135.8 (dd, J_{FF}=244.4, 26.3 Hz, 2F), −167.8 (s, 1F)

HR-APCI m/z calcd for C₂₆H₂₄F₅N₂O₂ [M+H]=491.1775, found 491.1752.

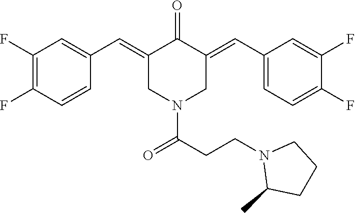 [SMILES: C[C@@H]1CCCN1CCC(=O)N1C/C(=C\c2ccc(F)c(F)c2)C(=O)/C(=C/c2ccc(F)c(F)c2)C1]

### 3,5-Bis((E)-3,4-difluorobenzylidene)-1-(3-((R)-2-methylpyrrolidin-1-yl)propanoyl)piperidin-4-one (JCS124)

¹H NMR (500 MHz, CDCl₃) δ 7.73 (br s, 2H), 7.32-7.15 (m, 6H), 4.93-4.67 (m, 4H), 3.21-2.98 (m, 2H), 2.67-2.41 (m, 4H), 2.22-2.14 (m, 1H), 1.99-1.90 (m, 1H), 1.88-1.65 (m, 2H), 1.52-1.44 (m, 1H), 1.12 (d, J=6.1 Hz, 3H).

¹³C NMR (126 MHz, CDCl₃) δ 185.9, 169.9, 151.0 (dd, J_{CF}=254.5 Hz, J_{C-CF}=12.6 Hz, 2C), 150.4 (dd, J_{CF}=250.7 Hz, J_{C-CF}=12.6 Hz, 2C), 136.2, 135.6, 131.9 (2C), 131.4 (d, J_{C-CF}=34.0 Hz, 2C), 127.1 (d, J_{C-CF}=45.3 Hz, 2C), 119.1 (dd, J_{C-C-CF}=39.0 Hz, J_{C-C-CF}=17.6 Hz, 2C), 118.1 (dd, J_{C-C-CF}=30.2 Hz, J_{C-C-CF}=17.6 Hz, 2C), 61.1, 53.7, 49.2, 46.4, 42.9, 32.1, 31.5, 21.4, 17.9. Due to slow amide rotation, one observes two absorptions for some carbons.

¹⁹F NMR (376 MHz, CDCl₃) δ −134.0 (dd, J_{FF}=154.1, 30.0 Hz, 2F), −135.8 (dd, J_{FF}=244.4, 26.3 Hz, 2F).

HR-APCI m/z calcd for C₂₇H₂₇F₄N₂O₂ [M+H]=487.2025, found 487.2003.

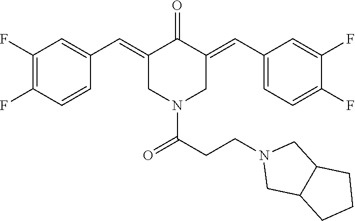 [SMILES: O=C1/C(=C/c2ccc(F)c(F)c2)CN(C(=O)CCN2CC3CCCC3C2)C/C1=C\c1ccc(F)c(F)c1]

### 3,5-Bis((E)-3,4-difluorobenzylidene)-1-(3-(hexahydrocyclopenta[c]pyrrol-2(1H)-yl)propanoyl)-piperidin-4-one (JCS125)

¹H NMR (500 MHz, CDCl₃) δ 7.73 (br s, 2H), 7.27-7.13 (m, 6H), 4.84 (s, 2H), 4.69 (s, 2H), 2.66 (br t, J=7.9 Hz, 2H), 2.60 (br t, J=7.6 Hz, 2H), 2.51 (m, 2H), 2.39 (br t, J=7.6 Hz, 2H), 1.88-1.85 (m, 2H), 1.63-1.40 (m, 4H), 1.33-1.24 (m, 2H).

¹³C NMR (126 MHz, CDCl₃) δ 186.0, 170.4, 151.0 (dd, J_{CF}=254.5 Hz, J_{C-CF}=12.6 Hz, 2C), 150.4 (dd, J_{CF}=250.7 Hz, J_{C-CF}=12.6 Hz, 2C), 136.4, 135.4, 132.2 (2C), 131.4 (d, J_{C-CF}=34.0 Hz, 2C), 127.1 (d, J_{C-CF}=45.3 Hz, 2C), 119.1 (dd, J_{C-C-CF}=39.0 Hz, J_{C-C-CF}=17.6 Hz, 2C), 118.0 (dd, J_{C-C-CF}=30.2 Hz, J_{C-C-CF}=17.6 Hz, 2C), 61.5, 51.1, 46.3, 43.2, 42.3, 32.4, 32.3, 25.7. Due to slow amide rotation, one observes two absorptions for some carbons.

¹⁹F NMR (376 MHz, CDCl₃) δ −134.1 (dd, J_{FF}=154.1, 30.0 Hz, 2F), −135.8 (dd, J_{FF}=244.4, 26.3 Hz, 2F).

HR-APCI m/z calcd for C₂₉H₂₉F₄N₂O₂ [M+H]=513.2186, found 513.2159.

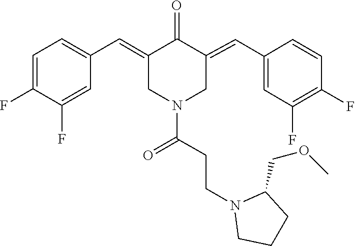 [SMILES: COC[C@@H]1CCCN1CCC(=O)N1C/C(=C\c2ccc(F)c(F)c2)C(=O)/C(=C/c2ccc(F)c(F)c2)C1]

### 3,5-Bis((E)-3,4-difluorobenzylidene)-1-(3-((S)-2-(methoxymethyl)pyrrolidin-1-yl)propanoyl)-piperidin-4-one (JCS126)

¹H NMR (500 MHz, CDCl₃) δ 7.70 (br s, 2H), 7.30-7.10 (m, 6H), 4.91-4.64 (m, 4H), 3.24 (m, 1H), 3.23 (s, 3H), 3.17 (dd, J=14.8, 7.4 Hz, 1H), 3.10-3.04 (m, 1H), 2.87-2.83 (m, 1H), 2.55-2.34 (m, 4H), 2.01-1.98 (m, 1H), 1.84-1.48 (m, 4H).

¹³C NMR (126 MHz, CDCl₃) δ 185.9, 170.2, 151.0 (dd, J_{CF}=254.5 Hz, J_{C-CF}=12.6 Hz, 2C), 150.4 (dd, J_{CF}=250.7 Hz, J_{C-CF}=12.6 Hz, 2C), 136.1, 135.3, 132.2 (2C), 131.4 (d, J_{C-CF}=34.0 Hz, 2C), 127.1 (d, J_{C-CF}=45.3 Hz, 2C), 119.1 (dd, J_{C-C-CF}=39.0 Hz, J_{C-C-CF}=17.6 Hz, 2C), 118.0 (dd, J_{C-C-CF}=30.2 Hz, J_{C-C-CF}=17.6 Hz, 2C), 75.5, 63.5, 59.0, 54.6, 50.9, 46.4, 42.9, 32.5, 28.0, 22.9. Due to slow amide rotation, one observes two absorptions for some carbons.

¹⁹F NMR (376 MHz, CDCl₃) δ −134.2 (dd, J_{FF}=154.1, 30.0 Hz, 2F), −135.8 (dd, J_{FF}=244.4, 26.3 Hz, 2F).

HR-APCI m/z calcd for C₂₈H₂₉F₄N₂O₃ [M+H]=517.2130, found 517.2108.

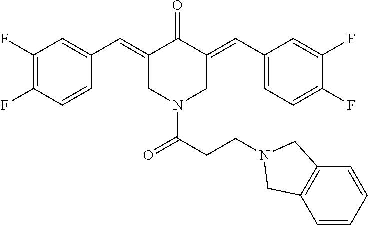 [SMILES: O=C1/C(=C/c2ccc(F)c(F)c2)CN(C(=O)CCN2Cc3ccccc3C2)C/C1=C\c1ccc(F)c(F)c1]

### 3,5-Bis((E)-3,4-difluorobenzylidene)-1-(3-(isoindolin-2-yl)propanoyl)piperidin-4-one (JCS127)

¹H NMR (500 MHz, CDCl₃) δ 7.73 (br s, 2H), 7.33-7.12 (m, 10H), 4.88 (s, 2H), 4.75 (s, 2H), 3.78 (s, 4H), 2.96 (t, J=7.5 Hz, 2H), 2.47 (t, J=7.5 Hz, 2H).

¹³C NMR (126 MHz, CDCl₃) δ 186.0, 170.4, 151.0 (dd, J_{CF}=254.5 Hz, J_{C-CF}=12.6 Hz, 2C), 150.4 (dd, J_{CF}=250.7 Hz, J_{C-CF}=12.6 Hz, 2C), 139.6 (2C), 136.5, 135.4, 132.2 (2C), 131.4 (d, J_{C-CF}=34.0 Hz, 2C), 127.1 (d, J_{C-CF}=45.3 Hz, 2C), 126.9 (2C), 122.3 (2C), 119.1 (dd, J_{C-C-CF}=39.0 Hz, J_{C-C-CF}=17.6 Hz, 2C), 118.0 (dd, J_{C-C-CF}=30.2 Hz, J_{C-C-CF}=17.6 Hz, 2C), 59.1, 51.4, 46.4, 43.2, 32.7. Due to slow amide rotation, one observes two absorptions for some carbons.

¹⁹F NMR (376 MHz, CDCl₃) δ −133.9 (dd, J_{FF}=154.1, 30.0 Hz, 2F), −135.7 (dd, J_{FF}=244.4, 26.3 Hz, 2F).

HR-APCI m/z calcd for C₃₀H₂₅F₄N₂O₂ [M+H]=521.1871, found 521.1846

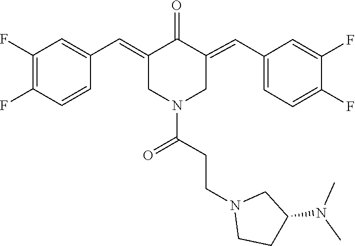 [SMILES: CN(C)[C@@H]1CCN(CCC(=O)N2C/C(=C\c3ccc(F)c(F)c3)C(=O)/C(=C/c3ccc(F)c(F)c3)C2)C1]

### 3,5-Bis((E)-3,4-difluorobenzylidene)-1-(3-((R)-3-(dimethylamino)pyrrolidin-1-yl)propanoyl)piper-idin-4-one (JCS128)

¹H NMR (500 MHz, CDCl₃) δ 7.74 (br s, 2H), 7.31-7.11 (m, 6H), 4.84 (s, 2H), 4.70 (s, 2H), 2.75-2.54 (m, 5H), 2.40-2.33 (m, 3H), 2.29-2.23 (m, 1H), 2.17 (s, 6H), 1.94-1.86 (m, 1H), 1.69-1.62 (m, 1H).

¹³C NMR (126 MHz, CDCl₃) δ 186.0, 170.4, 151.0 (dd, J_{CF}=254.5 Hz, J_{C-CF}=12.6 Hz, 2C), 150.4 (dd, J_{CF}=250.7 Hz, J_{C-CF}=12.6 Hz, 2C), 136.4, 135.4, 132.2 (2C), 131.4 (d, J_{C-CF}=34.0 Hz, 2C), 127.1 (d, J_{C-CF}=45.3 Hz, 2C), 119.1 (dd, J_{C-C-CF}=39.0 Hz, J_{C-C-CF}=17.6 Hz, 2C), 118.1 (dd, J_{C-C-CF}=30.2 Hz, J_{C-C-CF}=17.6 Hz, 2C), 65.2, 58.5, 53.3, 51.5, 46.4, 43.6, 43.2, 32.1, 28.8. Due to slow amide rotation, one observes two absorptions for some carbons.

¹⁹F NMR (376 MHz, CDCl₃) δ −134.0 (dd, J_{FF}=154.1, 30.0 Hz, 2F), −135.8 (dd, J_{FF}=244.4, 26.3 Hz, 2F).

HR-APCI m/z calcd for C₂₈H₃₀F₄N₃O₂ [M+H]=516.2294, found 516.2268.

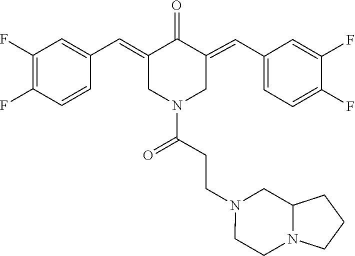 [SMILES: O=C1/C(=C/c2ccc(F)c(F)c2)CN(C(=O)CCN2CCN3CCCC3C2)C/C1=C\c1ccc(F)c(F)c1]

### 3,5-Bis((E)-3,4-difluorobenzylidene)-1-(3-(hexahydropyrrolo[1,2-a]pyrazin-2(1H)-yl)propanoyl)pi-peridin-4-one (JCS129)

¹H NMR (500 MHz, CDCl₃) δ 7.71 (br s, 2H), 7.29-7.09 (m, 6H), 4.83 (s, 2H), 4.69 (s, 2H), 3.04-2.88 (m, 2H), 2.75-2.56 (m, 4H), 2.37 (t, J=7.6 Hz, 2H), 2.20-1.87 (m, 4H), 1.83-1.62 (m, 4H), 1.33-1.28 (m, 1H).

¹³C NMR (126 MHz, CDCl₃) δ 185.9, 170.6, 151.0 (dd, J_{CF}=254.5 Hz, J_{C-CF}=12.6 Hz, 2C), 150.4 (dd, J_{CF}=250.7 Hz, J_{C-CF}=12.6 Hz, 2C), 136.4, 135.3, 132.2 (2C), 131.4 (d, J_{C-CF}=34.0 Hz, 2C), 127.1 (d, J_{C-CF}=45.3 Hz, 2C), 119.1 (dd, J_{C-C-CF}=39.0 Hz, J_{C-C-CF}=17.6 Hz, 2C), 118.1 (dd, J_{C-C-CF}=30.2 Hz, J_{C-C-CF}=17.6 Hz, 2C), 62.5, 57.7, 53.6, 53.2, 52.3, 51.3, 46.4, 43.2, 30.6, 27.5, 21.3. Due to slow amide rotation, one observes two absorptions for some carbons.

¹⁹F NMR (376 MHz, CDCl₃) δ −134.0 (dd, J_{FF}=154.1, 30.0 Hz, 2F), −135.8 (dd, J_{FF}=244.4, 26.3 Hz, 2F).

HR-APCI m/z calcd for C₂₉H₃₀F₄N₃O₂ [M+H]=528.2294, found 528.2268.

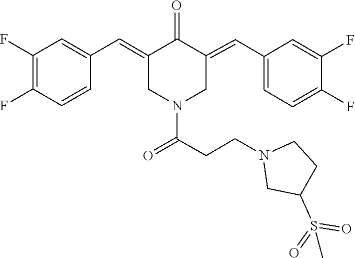 [SMILES: CS(=O)(=O)C1CCN(CCC(=O)N2C/C(=C\c3ccc(F)c(F)c3)C(=O)/C(=C/c3ccc(F)c(F)c3)C2)C1]

### 3,5-Bis((E)-3,4-difluorobenzylidene)-1-(3-(3-(methylsulfonyl)pyrrolidin-1-yl)propanoyl)piperidin-4-one (JCS130)

¹H NMR (500 MHz, CDCl₃) δ 7.73 (br s, 2H), 7.32-7.08 (m, 6H), 4.83 (s, 2H), 4.68 (s, 2H), 3.50-3.44 (m, 1H), 2.91-2.88 (m, 1H), 2.77 (s, 3H), 2.75-2.57 (m, 4H), 2.46-2.42 (m, 1H), 2.35 (t, J=7.1 Hz, 2H), 2.21-2.10 (m, 2H).

¹³C NMR (126 MHz, CDCl₃) δ 185.8, 170.0, 151.0 (dd, J_{CF}=254.5 Hz, J_{C-CF}=12.6 Hz, 2C), 150.4 (dd, J_{CF}=250.7 Hz, J_{C-CF}=12.6 Hz, 2C), 136.4, 135.3, 132.1 (2C), 131.4 (d, J_{C-CF}=34.0 Hz, 2C), 127.1 (d, J_{C-CF}=45.3 Hz, 2C), 119.1 (dd, J_{C-C-CF}=39.0 Hz, J_{C-C-CF}=17.6 Hz, 2C), 118.1 (dd, J_{C-C-CF}=30.2 Hz, J_{C-C-CF}=17.6 Hz, 2C), 61.4, 53.7, 53.4, 50.4, 46.4, 43.3, 38.4, 32.0, 25.8. Due to slow amide rotation, one observes two absorptions for some carbons.

¹⁹F NMR (376 MHz, CDCl₃) δ −134.0 (dd, J_{FF}=154.1, 30.0 Hz, 2F), −135.8 (dd, J_{FF}=244.4, 26.3 Hz, 2F).

HR-APCI m/z calcd for C₂₇H₂₇F₄N204S [M+H]=551.1647, found 551.1622.

### General Procedure E:

The compounds were generally prepared by reaction of the corresponding aldehydes, e.g., 3,4-difluorobenzaldehyde, with tert-butyl (4-oxocyclohexyl)carbamate in the present of 20% aq. sodium hydroxide to give N-tert-butyl (3,5-bis((E)-3,4-difluorobenzylidene)-4-oxocyclohexyl)carbamate. Treatment of the product with trifluoroacetic acid (TFA) caused deprotection of the Boc group. Benzoylation of 4-amino-2,6-bis((E)-3,4-difluorobenzylidene)cyclohexan-1-one with 4-(diethylcarbamoyl)-2-methyl-2H-indazole-7-carboxylic acid using standard peptide coupling reagents TBTU or EDC and HOAt afforded the N⁷-(3,5-bis((E)-3,4-difluorobenzylidene)-4-oxocyclohexyl)-N⁴,N⁴-diethyl-2-methyl-2H-indazole-4,7-dicarboxamide.

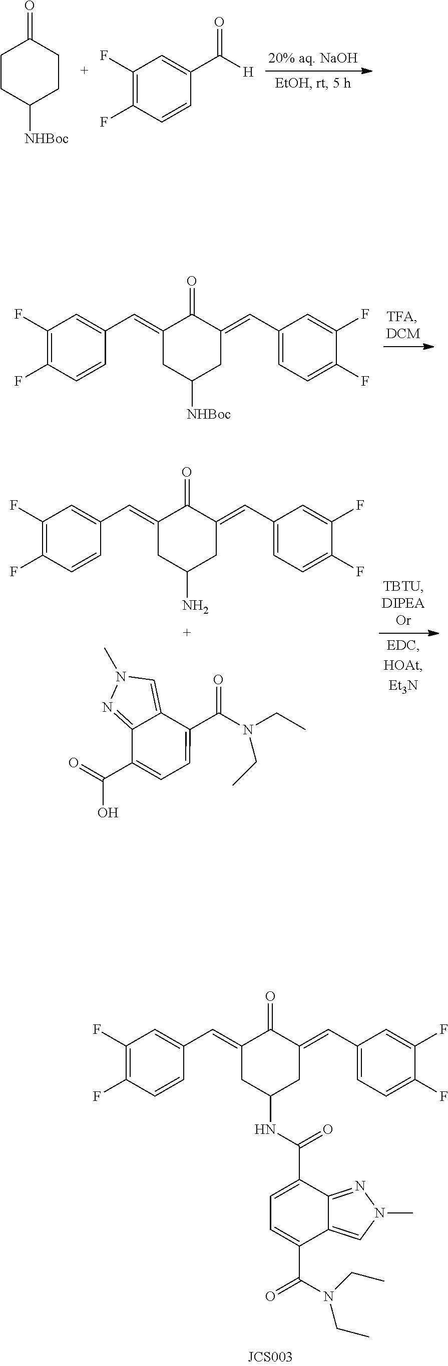

### Example JCS003

To the mixture of the N-tert-butyl (4-oxocyclohexyl)carbamate (213.28 mg, 1 mmol, 1.0 equiv.) and ethanol (1.0 mL) in a round bottom flask added drop-wise 20% aqueous sodium hydroxide (1.5 mL) and stirred for five minutes. To this mixture was added 3,4-difluorobenzaldehyde (355.3 mg, 2.5 mmol, 2.5 equiv.). The reaction mixture was then allowed to stir at 21° C. for 5 h. After 5 h the yellow precipitate thus obtained was filtered, washed with water, cold ethanol and dried to get the pure product (360 mg, 78% yield).

Trifluoroacetic acid (0.5 ml) was added to a solution of N-tert-butyl (3,5-bis((E)-3,4-difluorobenzylidene)-4-oxocyclohexyl)carbamate (230.7 mg, 0.5 mmol) in dichloromethane (5.0 ml) at 21° C. and stirred overnight at 21° C. Then, the solvent of the reaction solution was distilled off under reduced pressure and the resulting residue was poured into a 1N aqueous sodium hydroxide solution and extracted with ethyl acetate. The organic layer was washed with a saturated aqueous sodium chloride solution and then dried over anhydrous magnesium sulfate. The solvent was distilled off under reduced pressure to obtain 4-amino-2,6-bis((E)-3,4-difluorobenzylidene)cyclohexan-1-one.

The mixture of 4-amino-2,6-bis((E)-3,4-difluorobenzylidene)cyclohexan-1-one (180.7 mg, 0.5 mmol, 1.0 equiv.) and anhydrous diisopropylethylamine (261.2 μL, 1.5 mmol, 3.0 equiv.) in THF was maintained at 0° C. (ice bath). To this cooled mixture, 4-(diethylcarbamoyl)-2-methyl-2H-indazole-7-carboxylic acid (137.6 mg, 0.5 mmol, 1.0 equiv.) in 2.0 mL THF was added drop wise followed by and TBTU (240.8 mg, 0.75 mmol, 1.5 equiv.). After the complete addition of 4-(diethylcarbamoyl)-2-methyl-2H-indazole-7-carboxylic acid, the reaction mixture was slowly warmed up to 21° C. and stirred overnight. After completion of the reaction solvent was evaporated and the residue was stirred in sat aqueous NaHCO₃ for 5 min. The mixture was extracted with ethyl acetate three times. The organic layer was washed with a saturated aqueous sodium chloride solution and then dried over anhydrous sodium sulfate. The solvent was evaporated, followed by flash chromatography (gradient elution 75% EtOAc/Hexane-100% EtOAc) to give the desired product JCS003 (46.3 mg, 15% yield) as yellow solid.

### Synthesis of 4-(diethylcarbamoyl)-2-methyl-2H-indazole-7-carboxylic acid

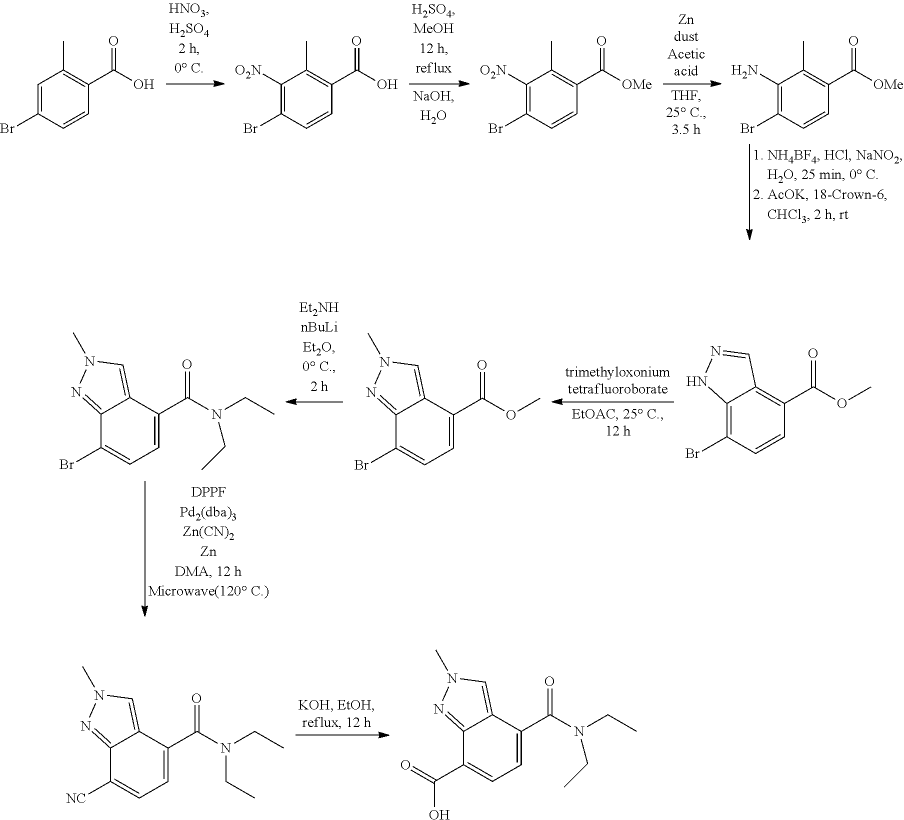

To a solution of nitric acid (3.34 mL, 75 mmol) in sulfuric acid (21 mL) was added a solution of 4-bromo-2-methylbenzoic acid (10.75 g, 50.0 mmol, 1.0 equiv) in concentrated sulfuric acid (72 mL) at 0° C., and the mixture was stirred at 0° C. for 30 min. The reaction solution was poured into ice water (250 mL), and the mixture was extracted with ethyl acetate. The extract was dried over anhydrous sodium sulfate and the solvent was evaporated under reduced pressure to give the title compound as a crudely purified product (13.0 g, purity 25%) which was used for next step without further purification.

A solution of the obtained crudely purified product of 4-bromo-2-methyl-3-nitrobenzoic acid (13.0 g, purity 25%) and sulfuric acid (1.5 mL, 0.64 mmol) in methanol (150 mL) was heated under reflux for 12 hr, and the solvent was evaporated under reduced pressure. The residue was diluted with ethyl acetate, washed with aqueous sodium hydroxide solution (20%), water and saturated brine, and dried over anhydrous sodium sulfate. The solvent was evaporated under reduced pressure to give methyl 4-chloro-2-methyl-3-nitrobenzoate as a crudely purified product. The crude was chromatographed with 10% EtOAC in hexane to give methyl 4-chloro-2-methyl-3-nitrobenzoate (7.81 g, 50% purity) as a crudely purified product.

Zn dust (67.81 g, 300.0 mmol, 10.0 equiv) was added portionwise to a solution of compound methyl 4-bromo-2-methyl-3-nitrobenzoate (7.81 g, 30 mmol, 1.0 equiv) in a mixture of THE (100 mL) and glacial acetic acid (6 mL). The reaction mixture was stirred at 25° C. for 3.5 hrs, diluted with EtOAc, and filtered through a pad of Celite. The cake was washed thoroughly with EtOAc. The filtrate was washed with saturated aqueous NaHCO₃. The aqueous layer was extracted with EtOAc. The combined extracts were dried over sodium sulfate, filtered and concentrated to yield the crude. The pure product was obtained by flash chromatography on silica gel (gradient elution 10% EtOAc/Hexane-50% EtOAc/Hexane) to give the desired product, methyl 3-amino-4-bromo-2-methylbenzoate (1.46 g, 20% yield), as a white solid.

To a solution of methyl 3-amino-4-bromo-2-methylbenzoate (8.5 g, 35 mmol) and ammonium tetrafluoroborate (4.76 g, 45.5 mmol, 1.3 equiv) in water (2.1 mL) and concentrated hydrochloric acid (18.5 mL, 227.5 mmol, 6.5 equiv) was added a solution of sodium nitrite (2.4 g, 35 mmol, 1 equiv) in water (18.5 mL) at 0° C. for 25 min, and the mixture was stirred for 35 min. The precipitated solid was collected by filtration, washed with ether (cold), and dried under reduced pressure. The obtained solid was dissolved in chloroform (105 mL), and 18-crown-6 (276.5 mg, 3 mol %) and potassium acetate (3.77 g mg, 38.5 mmol, 1.1 equiv) were added. The reaction mixture was stirred at room temperature for 12 hr. After 12 hr, solvent was evaporated and water was added to the reaction mixture, and the mixture was extracted with ethyl acetate. The extract was dried over anhydrous sodium sulfate, and the solvent was evaporated under reduced pressure. The pure product was obtained by flash chromatography on silica gel (gradient elution 10% EtOAc/Hexane-50% EtOAc/Hexane) to give the desired product, methyl 7-bromo-1H-indazole-4-carboxylate (4.46 g, 50% yield), as a white solid.

To a solution of methyl 7-bromo-1H-indazole-4-carboxylate (4.46 g, 17.6 mmol) in ethyl acetate (60 mL) was added trimethyloxonium tetrafluoroborate (3.9 g, 26.4 mmol, 1.5 equiv) at room temperature, and the mixture was stirred for 12 hr. This reaction solution was diluted with water, and the mixture was extracted with ethyl acetate. The extract was dried over anhydrous sodium sulfate and the solvent was evaporated under reduced pressure to give methyl 7-bromo-2-methyl-2H-indazole-4-carboxylate as desired product in quantitative yield and was used for next step without further purification.

An oven-dried schlenk flask under argon condition, diethylamine (387.5 μL, 3.75 mmol, 1.5 equiv) and Et₂O (2.5 mL) was added, and the reaction mixture was cooled to 0° C. using ice bath. The nBuLi (1.5 mL, 3.75 mmol) was added dropwise and stir the mixture of 30 min. After 30 min, methyl 7-bromo-2-methyl-2H-indazole-4-carboxylate (672.5 mg, 2.5 mmol) in Et₂O (2.5 mL) was added to the reaction mixture dropwise. The reaction was further stirred for 2 h. After 2 hr, this reaction solution was diluted with water, and the mixture was extracted with ethyl acetate. The extract was dried over anhydrous sodium sulfate and the solvent was evaporated under reduced pressure to obtain the crude. The pure product was obtained by flash chromatography on silica gel (gradient elution 50% EtOAc/Hexane-100% EtOAc) to give the desired product, 7-bromo-N,N-diethyl-2-methyl-2H-indazole-4-carboxamide (542.8 mg, 70% yield), as a yellow oil.

To a microwave vial equipped with a stir bar was added 7-bromo-N,N-diethyl-2-methyl-2H-indazole-4-carboxamide (888.48 mg, 3 mmol, 1 equiv), 1,1′-Bis(diphenylphosphino)ferrocene (165 mg, 10 mol %), Pd₂(dba)₃ (136.5 mg, 5 mol %), Zn (36 mg, 20 mol %), Zn(CN)₂ (351 mg, 3 mmol, 1 equiv) in DMA (5 mL, degassed). The mixture was heated in microwave at 120° C. for 12 h, after which time diluted with ethyl acetate (15 mL), washed with saturated aqueous brine (10 mL), dried over sodium sulfate and evaporated to give 7-cyano-N,N-diethyl-2-methyl-2H-indazole-4-carboxamide as desired product which was used for next step without further purification.

7-cyano-N,N-diethyl-2-methyl-2H-indazole-4-carboxamide (768.9 mg, 3.0 mmol, 1.0 equiv) was dissolved in 2.5 mL ethanol and added to a solution of potassium hydroxide (841.5 mg, 15 mmol, 5 equiv) in 2.5 mL water. The mixture was heated under reflux for 12 h. The ethanol was removed in vacuo and the aqueous solution was acidified with cone. HCl at 5° C. The solid was collected, treated with cold water, filtered and dried at 55-60° C. in vacuo to give 4-(diethylcarbamoyl)-2-methyl-2H-indazole-7-carboxylic acid hydrochloride as white sold (743.3 mg, 90% yield) which was enough pure to be used for next step.

The following compounds were synthesized by procedure E: JCS003, JCS006.

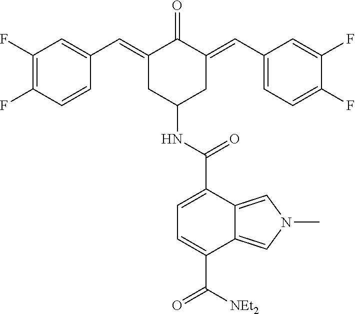

### N⁷-(3,5-bis((E)-3,4-difluorobenzylidene)-4-oxocyclohexyl)-N⁴,N⁴-diethyl-2-methyl-2H-indazole-4,7-dicarboxamide (JCS003)

¹H NMR (500 MHz, CDCl₃) δ 9.41 (d, J=7.3 Hz, 1H), 8.14 (d, J=7.2 Hz, 1H), 8.02 (s, 1H), 7.84 (s, 2H), 7.31-7.15 (m, 6H), 7.13 (d, J=7.2 Hz, 1H), 4.72-4.66 (m, 1H), 4.17 (s, 3H), 3.65-3.54 (m, 1H), 3.31-3.18 (m, 6H), 1.28 (t, J=6.3 Hz, 3H), 1.07 (t, J=6.3 Hz, 3H). ¹³C NMR (126 MHz, CDCl₃) δ 188.2, 168.8, 164.6, 150.6 (dd, J_{CF}=253.2 Hz, J_{C-CF}=12.6 Hz, 2C), 150.2 (dd, J_{CF}=249.4 Hz, J_{C-CF}=12.6 Hz, 2C), 146.1, 137.7 (2C), 133.3 (2C), 132.8, 132.4 (dd, J_{C-C-CF}=6.3 Hz, J_{C-C-CF}=3.7 Hz, 2C) 128.7 (2C), 127.1 (dd, J_{C-C-CF}=6.3 Hz, J_{C-C-CF}=3.7 Hz, 2C), 125.0, 121.5, 121.1, 119.3 (2C), 119.0 (d, J_{C-CF}=17.6 Hz, 2C), 117.7 (d, J_{C-CF}=17.6 Hz, 2C), 44.4, 43.4, 40.7, 39.5, 33.7 (2C), 14.7, 13.0. Due to formation of rotamers, we can see two more carbon peaks in up field position. These up field signals are more distinct for JCS006. ¹⁹F NMR (376 MHz, CDCl₃) δ −135.4 (d, J_{FF}=22.5 Hz, 2F), −136.7 (d, J_{FF}=18.8 Hz, 2F). HR-APCI m/z calcd for C₃₄H₃₁F₄N₄O₃ [M+H]=619.2368, found 619.2457.

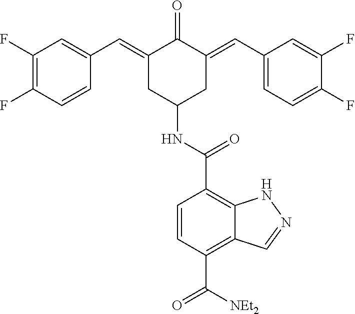

### N⁷-(3,5-bis((E)-3,4-difluorobenzylidene)-4-oxocyclohexyl)-N⁴,N⁴-diethyl-1H-indazole-4,7-dicarboxamide (JCS006)

¹H NMR (500 MHz, CDCl₃) δ 9.16 (d, J=7.4 Hz, 1H), 8.14 (d, J=7.2 Hz, 1H), 7.84 (s, 2H), 7.32-7.14 (m, 7H), 7.07 (d, J=7.2 Hz, 1H), 4.71-4.67 (m, 1H), 4.10 (s, 3H), 3.76-3.47 (m, 2H), 3.33-3.21 (m, 4H), 3.15-3.05 (m, 2H), 1.30 (t, J=7.1 Hz, 3H), 1.00 (t, J=7.1 Hz, 3H). ¹³C NMR (126 MHz, CDCl₃) δ 188.1, 166.9, 164.1, 150.6 (dd, J_{CF}=257.2 Hz, J_{C-CF}=12.6 Hz, 2C), 150.2 (dd, J_{CF}=249.4 Hz, J_{C-CF}=12.6 Hz, 2C), 144.9, 137.7 (2C), 133.1 (2C), 132.9, 132.4 (dd, J_{C-C-CF}=6.3 Hz, J_{C-C-CF}=3.7 Hz, 2C) 130.0 (2C), 127.1 (dd, J_{C-C-CF}=6.3 Hz, J_{C-C-CF}=3.7 Hz, 2C), 121.9, 121.4, 119.8 (2C), 119.0 (d, J_{C-CF}=17.6 Hz, 2C), 117.7 (d, J_{C-CF}=17.6 Hz, 2C), 115.9, 44.3, 43.0, 39.3, 38.0, 33.6, 14.0, 12.7. Due to formation of rotamers, we can see two more carbon peaks in up field position. ¹⁹F NMR (376 MHz, CDCl₃) δ −134.7 (d, J_{FF}=18.8 Hz, 2F), −136.3 (d, J_{FF}=18.8 Hz, 2F). HR-APCI m/z calcd for C₃₃H₂₇F₄N₄O₃ [M−H]=603.2024, found 603.2025.

### General Procedure F:

The compounds were generally prepared by palladium catalyzed Suzuki cross coupling of 4,4,5,5-tetramethyl-2-(1,4-dioxaspiro[4.5]dec-7-en-8-yl)-1,3,2-dioxaborolane with 7-bromo-N,N-diethyl-2-methyl-2H-indazole-4-carboxamide to give N,N-diethyl-2-methyl-7-(1,4-dioxaspiro[4.5]dec-7-en-8-yl)-2H-indazole-4-carboxamide. Hydrogenation of N,N-diethyl-2-methyl-7-(1,4-dioxaspiro[4.5]dec-7-en-8-yl)-2H-indazole-4-carboxamide gave N,N-diethyl-2-methyl-7-(4-oxocyclohexyl)-2H-indazole-4-carboxamide which was subjected to aldol condensation with 3,4-difluorobenzaldehyde to give 7-(3,5-bis((E)-3,4-difluorobenzylidene)-4-oxocyclohexyl)-N,N-diethyl-2-methyl-2H-indazole-4-carboxamide

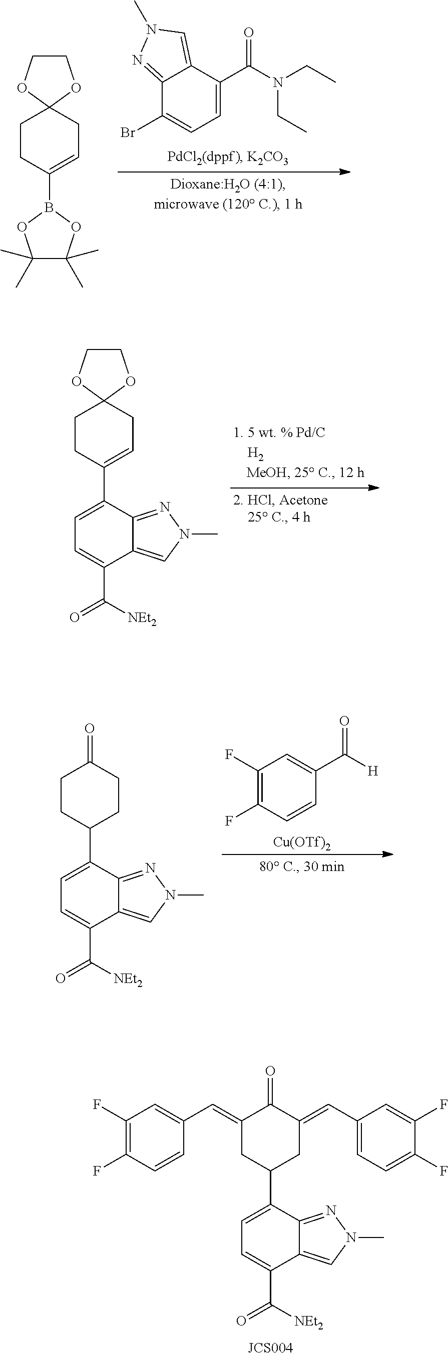

### Example JCS004

To a microwave vial equipped with a stir bar was added 4,4,5,5-tetramethyl-2-(1,4-dioxaspiro[4.5]dec-7-en-8-yl)-1,3,2-dioxaborolane (532 mg, 2 mmol, 2.0 equiv), 7-bromo-N,N-diethyl-2-methyl-2H-indazole-4-carboxamide (309 mg, 1.0 mmol, 1.0 equiv.), PdCl₂(dppf) (146 mg, 20 mol %), K₂CO₃ (414 mg, 3 mmol, 3.0 equiv) and Dioxane:H₂O (4:1) (15 mL). The mixture was heated in microwave at 120° C. for 1 h, after which time diluted with ethyl acetate (15 nmL), washed with saturated aqueous brine (10 mL), dried over sodium sulfate and evaporated to give crude. The pure product was obtained by flash chromatography on silica gel (gradient elution 75% EtOAc/Hexane-10% Methanol/EtOAc) to give the desired product, N,N-diethyl-2-methyl-7-(1,4-dioxaspiro[4.5]dec-7-en-8-yl)-2H-indazole-4-carboxamide (336.1 mg, 91% yield), as a brown solid.

In a stainless steel high pressure reactor vessel, N,N-diethyl-2-methyl-7-(1,4-dioxaspiro[4.5]dec-7-en-8-yl)-2H-indazole-4-carboxamide (336.1 mg, 0.91 mmol, 1.0 equiv.), MeOH (10 mL), 5 wt. % Pd/C (106.4 mg, 1 mmol, 1.1 equiv) were added and stirred under high pressure (20 bar) of hydrogen at 21° C. for 12 h. Then, the resulting solution was filtered through a pad of Celite, concentrated to give the crude. The pure product was obtained by flash chromatography on silica gel (gradient elution 10% EtOAc/Hexane-50% EtOAc/Hexane) to give the desired product, N,N-diethyl-2-methyl-7-(1,4-dioxaspiro[4.5]decan-8-yl)-2H-indazole-4-carboxamide (293.4 mg, 79% yield), as a yellow oil. Thus obtained N,N-diethyl-2-methyl-7-(1,4-dioxaspiro[4.5]decan-8-yl)-2H-indazole-4-carboxamide was treated with 1N HCl (2 mL) in acetone (4 mL) and stirred at 21° C. for 4 hr. After 4 h, acetone was evaporated and the reaction was neutralized with saturated NaHCO₃ solution. The residue was partitioned between ethyl acetate and water. The organic layer was washed with brine, dried over anhydrous Na₂SO₄, filtered and concentrated to give N,N-diethyl-2-methyl-7-(4-oxocyclohexyl)-2H-indazole-4-carboxamide (quantitative yield), as yellow solid which was pure enough to move for next step without further purification.

An oven-dried schlenk flask under argon condition, N,N-diethyl-2-methyl-7-(4-oxocyclohexyl)-2H-indazole-4-carboxamide (167.7 mg, 0.5 mmol, 1.0 equiv), 3,4-difluorobenzaldehyde (177.6 mg, 1.25 mmol, 2.5 equiv), copper(II) triflate (180.3 mg, 0.5 mmol, 0.5 equiv) were added, and the reaction mixture was stirred at 80° C. for 30 min. After 30 mins, the reaction mixture solidified to dark brown crude solid. The pure product was obtained by flash chromatography on silica gel (gradient elution 50% EtOAc/Hexane-100% EtOAc) to give the desired product, 7-(3,5-bis((E)-3,4-difluorobenzylidene)-4-oxocyclohexyl)-N,N-diethyl-2-methyl-2H-indazole-4-carboxamide (115.1 mg, 40% yield), as a yellow solid.

The following compounds were synthesized by procedure E: JCS004, JCS005.

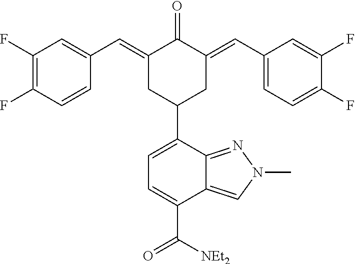

### 7-(3,5-bis((E)-3,4-difluorobenzylidene)-4-oxocyclohexyl)-N,N-diethyl-2-methyl-2H-indazole-4-carboxamide (JCS004)

¹H NMR (500 MHz, CDCl₃) δ 7.96 (s, 1H), 7.73 (s, 2H), 7.30-7.24 (m, 2H), 7.21-7.07 (m, 6H), 7.02 (d, J=7.0 Hz, 1H), 4.17 (s, 3H), 3.67-3.61 (m, 1H), 3.60-3.32 (m, 8H), 1.21 (t, J=7.1 Hz, 6H).

¹³C NMR (126 MHz, CDCl₃) δ 189.2, 169.7, 150.4 (dd, J_{CF}=252.0 Hz, J_{C-CF}=12.6 Hz, 2C), 150.1 (dd, J_{CF}=249.4 Hz, J_{C-CF}=12.6 Hz, 2C), 147.8, 136.0 (2C), 135.5 (2C), 134.4 (2C), 132.8 (dd, J_{C-C-CF}=6.3 Hz, J_{C-C-CF}=3.7 Hz, 2C), 127.7 (2C), 127.1 (dd, J_{C-C-CF}=6.3 Hz, J_{C-C-CF}=3.7 Hz, 2C), 124.1, 121.5, 120.7, 119.5 (2C), 118.9 (d, J_{C-CF}=17.6 Hz, 2C), 117.4 (d, J_{C-CF}=17.6 Hz, 2C), 43.5, 40.6, 39.3, 36.9, 33.8 (2C), 14.3, 13.2. Due to formation of rotamers, we can see two more carbon peaks in up field position. ¹⁹F NMR (376 MHz, CDCl₃) δ −136.1 (d, J_{FF}=18.8 Hz, 2F), −137.1 (d, J_{FF}=18.8 Hz, 2F).

HR-APCI m/z calcd for C₃₃H₃₀F₄N₃O₂ [M+H]=576.2256, found 576.2260.

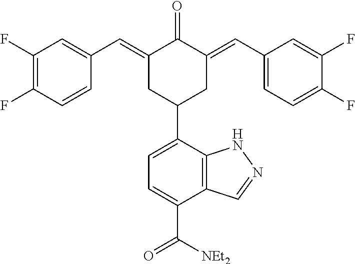

### 7-(3,5-bis((E)-3,4-difluorobenzylidene)-4-oxocyclohexyl)-N,N-diethyl-1H-indazole-4-carboxamide (JCS005)

¹H NMR (500 MHz, CDCl₃) δ 12.28 (s, 1H), 8.09 (s, 1H), 7.66 (s, 2H), 7.32 (d, J=7.0 Hz, 1H), 7.16 (d, J=7.0 Hz, 1H), 7.17-3.01 (m, 6H), 3.70-3.62 (m, 2H), 3.53-3.48 (m, 1H), 3.30 (bd, J=15.0 Hz, 4H), 3.17-3.11 (m, 2H), 1.32 (t, J=7.1 Hz, 3H), 1.11 (t, J=7.1 Hz, 3H).

¹³C NMR (126 MHz, CDCl₃) δ 189.1, 169.5, 150.6 (dd, J_{CF}=254.5 Hz, J_{C-CF}=12.6 Hz, 2C), 150.1 (dd, J_{CF}=249.4 Hz, J_{C-CF}=12.6 Hz, 2C), 139.2, 136.6 (2C), 134.8 (2C), 134.2 (2C), 132.0 (dd, J_{C-C-CF}=6.3 Hz, J_{C-C-CF}=3.7 Hz, 2C), 128.6 (2C), 127.7, 127.2 (dd, J_{C-C-CF}=6.3 Hz, J_{C-C-CF}=3.7 Hz, 2C), 122.6, 121.3, 119.1 (d, J_{C-CF}=17.6 Hz, 2C), 119.0 (2C), 117.6 (d, J_{C-CF}=17.6 Hz, 2C), 43.4, 39.4, 36.1, 34.1 (2C), 14.6, 13.1. Due to formation of rotamers, we can see two more carbon peaks in up field position.

¹⁹F NMR (376 MHz, CDCl₃) δ −136.1 (d, J_{FF}=18.8 Hz, 2F), −137.1 (d, J_{FF}=18.8 Hz, 2F).

HR-APCI m/z calcd for C₃₂H₂₈F₄N₃O₂ [M+H]=562.2112, found 562.2133.

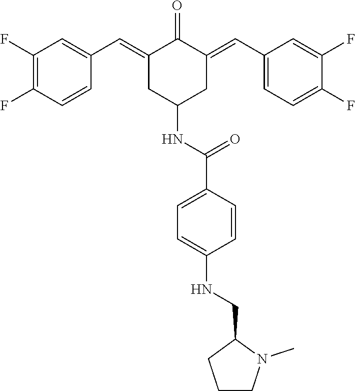 [SMILES: CN1CCC[C@H]1CNc1ccc(C(=O)NC2C/C(=C\c3ccc(F)c(F)c3)C(=O)/C(=C/c3ccc(F)c(F)c3)C2)cc1]

### N-(3,5-Bis((E)-3,4-difluorobenzylidene)-4-oxocyclohexyl)-4-((((S)-1-methylpyrrolidin-2-yl)methyl)-amino)benzamide (JCS160)

¹H NMR (500 MHz, CDCl₃) δ 7.78 (s, 2H), 7.52 (d, J=8.6 Hz, 2H), 7.27-7.16 (m, 6H), 6.53 (d, J=8.7 Hz, 2H), 6.06 (d, J=7.3 Hz, 1H), 4.69 (br s, 1H), 4.52-4.45 (m, 1H), 3.27-3.17 (m, 3H), 3.15-3.10 (m, 2H), 3.04 (dd, J=16.6, 7.7 Hz, 2H), 2.52-2.47 (m, 1H), 2.31 (s, 3H), 2.29-2.24 (m, 1H), 1.96-1.67 (m, 4H).

¹³C NMR (126 MHz, CDCl₃) δ 188.1, 167.0, 151.8, 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 137.7 (2C), 133.0 (2C), 132.3 (dd, J_{C-C-CF}=6.3 Hz, J_{C-C-CF}=3.7 Hz, 2C), 128.7 (2C), 127.1 (dd, J_{C-C-CF}=6.3 Hz, J_{C-C-CF}=3.7 Hz, 2C), 121.4, 119.1 (d, J_{C-CF}=17.6 Hz, 2C), 117.7 (d, J_{C-CF}=17.6 Hz, 2C), 111.8 (2C), 64.2, 57.4, 44.4, 43.8, 40.4, 33.9, 28.6, 22.7.

¹⁹F NMR (376 MHz, CDCl₃) δ −135.2 (d, J_{FF}=26.3 Hz, 2F), −136.6 (d, J_{FF}=26.3 Hz, 2F).

HR-APCI m/z calcd for C₃₃H₃₂F₄N₃O₂ [M+H]=578.2420, found 578.2425.

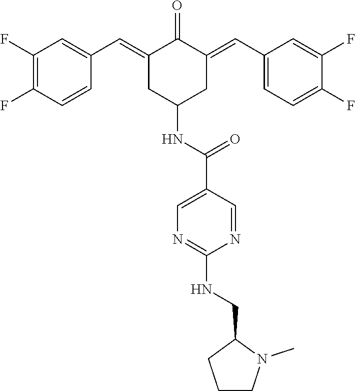 [SMILES: CN1CCC[C@H]1CNc1ncc(C(=O)NC2C/C(=C\c3ccc(F)c(F)c3)C(=O)/C(=C/c3ccc(F)c(F)c3)C2)cn1]

### N-(3,5-Bis((E)-3,4-difluorobenzylidene)-4-oxocyclohexyl)-2-((((S)-1-methylpyrrolidin-2-yl)methyl)-amino)pyrimidine-5-carboxamide (JCS161)

¹H NMR (500 MHz, CDCl₃) δ 8.60 (s, 2H), 7.77 (s, 2H), 7.26-7.15 (m, 6H), 6.19 (br s, 1H), 4.53-4.46 (m, 1H), 3.69-3.64 (m, 1H), 3.35-3.32 (m, 1H), 3.24 (br d, J=14.0 Hz, 2H), 3.08 (m, 3H), 2.54-2.42 (m, 1H), 2.32 (s, 3H), 2.26-2.21 (m, 1H), 1.92-1.57 (m, 4H).

¹³C NMR (126 MHz, CDCl₃) δ 187.8, 164.3, 163.5, 157.8, 151.8 (2C), 150.7 (dd, J_{CF}=253.2 Hz, J_{C-CF}=12.6 Hz, 2C), 150.3 (dd, J_{CF}=249.4 Hz, J_{C-CF}=12.6 Hz, 2C), 138.1 (2C), 132.6 (2C), 132.1 (dd, J_{C-C-CF}=6.3 Hz, J_{C-C-CF}=3.7 Hz, 2C), 127.2 (dd, J_{C-C-CF}=6.3 Hz, J_{C-C-CF}=3.7 Hz, 2C), 119.0 (d, J_{C-CF}=17.6 Hz, 2C), 117.7 (d, J_{C-CF}=17.6 Hz, 2C), 116.3, 64.0, 57.3, 44.5, 42.3, 40.4, 33.7, 28.4, 22.6. Due to slow amide rotation, one observes two absorptions for some carbons.

¹⁹F NMR (376 MHz, CDCl₃) δ −134.9 (d, J_{FF}=26.3 Hz, 2F), −136.4 (d, J_{FF}=26.3 Hz, 2F).

HR-APCI m/z calcd for C₃₁H₃₀F₄N₅O₂ [M+H]=580.2324, found 580.2330.

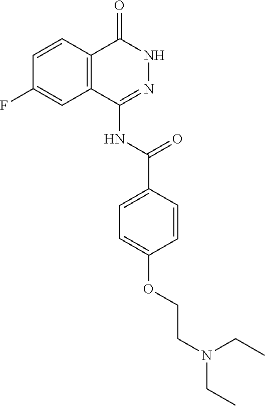 [SMILES: CCN(CC)CCOc1ccc(C(=O)Nc2n[nH]c(=O)c3ccc(F)cc23)cc1]

### 4-(2-(Diethylamino)ethoxy)-N-(7-fluoro-4-oxo-3,4-dihydrophthalazin-1-yl)benzamide (JCS162)

¹H NMR (500 MHz, CDCl₃) δ 12.48 (br s, 1H), 7.80 (d, J=8.3 Hz, 2H), 7.61 (d, J=8.1 Hz, 1H), 7.28-7.22 (m, 2H), 6.72 (d, J=8.5 Hz, 2H), 4.36 (t, J=5.0 Hz, 2H), 3.43 (t, J=5.0 Hz, 2H), 3.20 (q, J=7.2 Hz, 4H), 1.27 (t, J=7.2 Hz, 6H).

¹³C NMR (126 MHz, CDCl₃) δ 169.8 (2C), 165.8, 160.5, 143.3, 131.6 (2C), 127.8, 126.1, 124.9, 124.0, 118.6, 113.6 (2C), 110.9, 63.7, 50.8, 47.0, 12.1.

¹⁹F NMR (376 MHz, CDCl₃) δ −150.9 (s, F)

HR-APCI m/z calcd for C_{21′}H₂₄FN₄O₃ [M+H]=399.1853, found 399.1826.

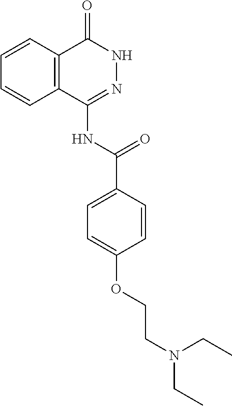 [SMILES: CCN(CC)CCOc1ccc(C(=O)Nc2n[nH]c(=O)c3ccccc23)cc1]

### 4-(2-(Diethylamino)ethoxy)-N-(4-oxo-3,4-dihydrophthalazin-1-yl)benzamide (JCS163)

¹H NMR (500 MHz, CDCl₃) δ 8.20 (d, J=8.9 Hz, 2H), 8.08 (d, J=8.2 Hz, 1H), 7.57-7.40 (m, 3H), 7.05 (d, J=8.9 Hz, 2H), 4.18 (t, J=6.0 Hz, 2H), 2.94 (t, J=6.1 Hz, 2H), 2.68 (q, J=7.1 Hz, 4H), 1.09 (t, J=7.1 Hz, 6H).

¹³C NMR (126 MHz, CDCl₃) δ 164.7 (2C), 162.4, 143.6, 133.1 (2C), 129.0 (2C), 128.7, 124.8, 120.5, 116.6, 115.1 (2C), 108.5, 67.2, 51.5, 47.9, 11.8.

HR-APCI m/z calcd for C_{21′}H₂₅N₄O₃ [M+H]=381.1932, found 381.1921.

### Example 2: Exemplary Biological Activity of Compounds of the Disclosure

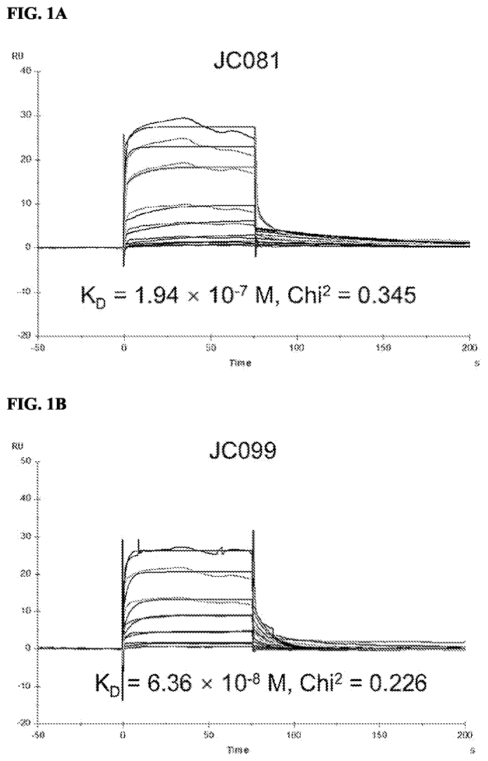

### JC081 and JC099 Directly Bind to PARP1 (FIG. 1)

To evaluate the binding affinity of JC081 and JC099 for PARP1, Biacore T100, which is a precise surface plasmon resonance (SPR) system to generate high-quality molecular interaction data, was used to analyze the kinetic parameters between JC081/99 and PARP1. Purified PARP1 enzymes were immobilized to a CM5 sensor chip. The concentration set of JC081/99 was 4 nM-8 μM at a twofold dilution. As shown in FIGS. 1A & 1B, the equilibrium dissociation constant K_{D} of JC081 and JC099 binding to PARP1 is 1.94×10-7 and 6.36×10-8 M, respectively; this indicates that JC081 and JC099 have high affinity for PARP1.

### JC081 and JC099 Inhibit PARP1 Catalytic Activity (FIG. 2)

PARP1 is known to catalyze the NAD-dependent addition of poly(ADP-ribose) to histones. Clinical PARP inhibitors, such as Olaparib (OLA) and Talazoparib (TALA), have high potency in inhibiting PARP1 catalytic activity. In order to assess the effect of JC099 on PARP1 catalytic activity, a universal chemiluminescent PARP assay kit (4676-096-K, Trevigen, Gaithersburg, MD, USA) was used, which detects biotinylated poly (ADP-ribose) deposited by PARP-1 onto immobilized histones, to evaluate the inhibition of JC099 on PARP1 catalytic activity. OLA was used as positive control. As shown in FIG. 2, JC099 suppressed PARP1 activity in a concentration-dependent manner, albeit less than OLA. Importantly, JC099 did not significantly inhibit PARP2 activity, suggesting that JC099 mainly target PARP1.

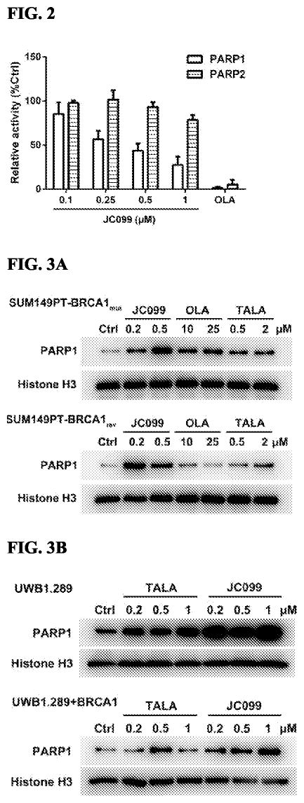

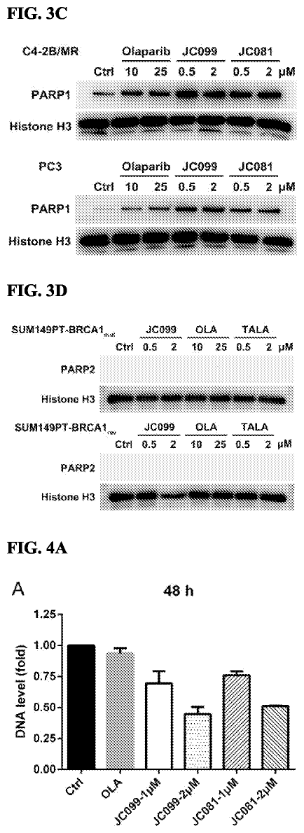

### JC081 and JC099 Potently Trap PARP1 on DNA Lesions (FIG. 3)

Compared with PARP1 catalytic activity inhibition, growing evidence indicates that PARP-trapping on chromatin is a more toxic inducer of cytotoxicity. The trapped PARP1 forms a toxic lesion and stalls the progress of replication forks. The replication fork collapses in BRCA1/2-deficient cancer cells, and then results in cell death. To evaluate the effect of JC099 and JC081 on PARP1 trapping, a subcellular protein fractionation kit (Thermo Scientific, 78840) was used to separate chromatin-bound extracts from JC099- or JC081-treated cells by stepwise centrifugation. OLA and TALA, the most efficient PARP1 trapper among clinical PARP inhibitors, were used for comparison. Six cancer cell lines were used in this assay, including two breast cancer cell lines, BRCA1-mutunt SUM149PT-BRCA1ₘᵤₜ and SUM149PT-BRCA1ᵣₑᵥ where deleterious BRCA1 mutation is reverted to form a functional BRCA1, two ovarian cancer cell lines, BRCA1-null UWB1.289 and UWB1.289+BRCA1, a stable cell line in which wild-type BRCA1 function has been restored, and two prostate cancer cell lines, enzalutamide resistant C4-2B/MR which is homologous recombination (HR)-deficient due to Rad51 mutation, and enzalutamide insensitive PC3 which is HR-proficient. OLA and TALA greatly increased accumulation of PARP1 protein in the chromatin-bound fraction in HR-deficient SUM149PT-BRCA1ₘᵤₜ (FIG. 3A), UWB1 (FIG. 3B) and C4-2B/MR (FIG. 3C) cells, compared with HR-proficient SUM149PT-BRCA1ᵣₑᵥ (FIG. 3A), UWB1+BRCA1 (FIG. 3B) and PC3 (FIG. 3C) cells. However, JC099 was more efficient at trapping PARP-1 on damaged DNA than OLA and TALA in both HR-deficient and -proficient breast, ovarian, and prostate cancer cell lines. JC081 also exhibited high PARP1-trapping potency, which is close to JC099, in both C4-2B/MR and PC3 cells (FIG. 3C). However, it was found that JC099 could not trap PARP2 in chromatin (FIG. 3D). Taken together, our results suggest that JC081 and JC099 mainly inhibit PARP1, but not PARP2.

### JC099 and JC081 Inhibit HR (FIG. 3A)

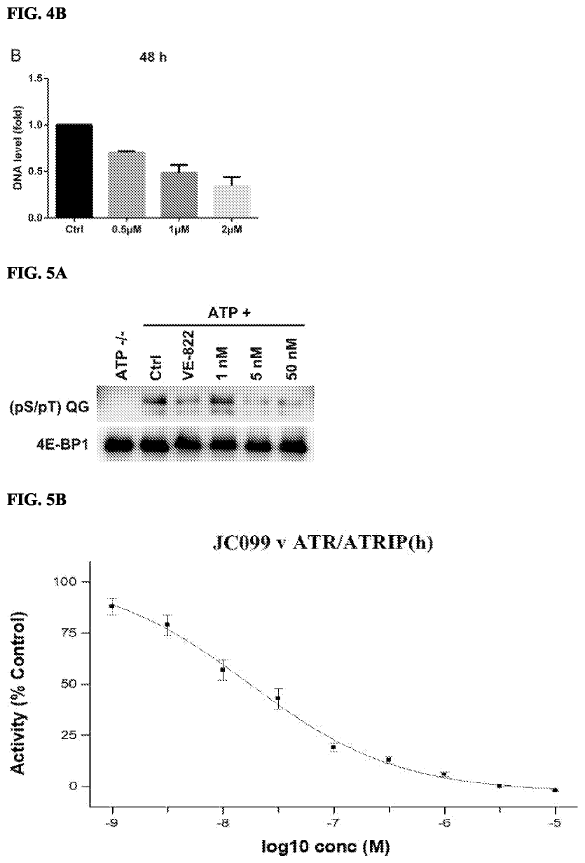

PARP-trapping can be repaired by HR-mediated pathways, whereas JC099 and JC081 can trap PARP-1 on damaged DNA in HR-proficient cancer cells. Therefore, it is hypothesized that JC099 and JC081 might affect HR. A homologous recombination assay kit (35600, Norgen Biotek Corporation, Thorold, ON Canada) was used to quantitatively analyze the HR efficiency through qPCR. Briefly, the lacZα coding region of the positive control plasmid was mutated and two plasmids with different mutations were generated. Therefore, the two plasmids containing different defective lacZα cassettes can form a functional lacZα cassette through intermolecular HR. The two plasmids were co-transfected into SUM149PT-BRCA1ᵣₑᵥ and PC3 cells, followed by the treatment of JC099 or JC081 for 48 h, and then the DNA was isolated to assay HR efficiency by qPCR. As shown in FIG. 4A, OLA didn't affect HR in PC3 cells, while JC099 and JC081 strikingly suppressed HR, and reduced about 50% of HR response. JC099 also greatly reduced HR response in a dose-dependent manner in SUM149PT-BRCA1ᵣₑᵥ cells (FIG. 4B).

### JC099 Inhibits ATR

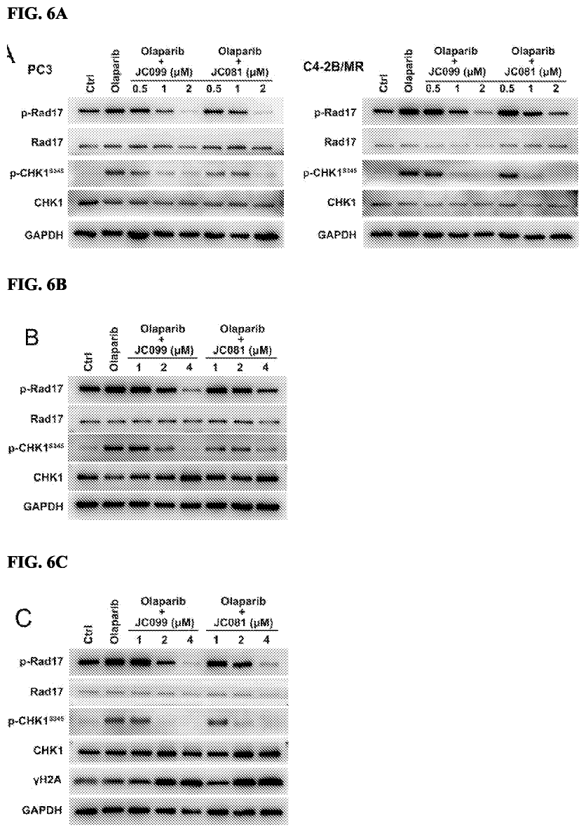

PARP inhibitor-resistant cells are increasingly dependent on ATR for survival, and ATR inhibition disrupts restored HR and fork protection pathways in PARP inhibitor resistant BRCA-deficient cancer cells. For ATR kinase in vitro assays, ATR was immunoprecipitated with 3 μg of anti-ATR antibody (Bethyl Laboratories, A300-137A) from 1 mg of cell lysate. Purified ATR was then used to phosphorylate 4E-BP1 (Sigma-Aldrich, SRP0253), also named as PHAS-I, a specific substrate of ATR, in the presence of increasing concentrations of JC099. 50 nM of VE-822, a specific ATR inhibitor, was used as positive control. JC099 dramatically decreased ATR activity in SUM149PT-BRCA1ᵣₑᵥ and PC3 cells in a dose-dependent manner (FIG. 5A). In vitro kinase assays showed that JC099 inhibited ATR activity with IC50 17 nM (FIG. 5B), which is lower than reported IC50 of VE-822, 19 nM. The IC50 was determined by the enzyme profiling service of Eurofins Cerep (France). The effect of JC099 and JC081 on ATR-mediated signaling pathway was also detected. The phosphorylation of CHK1 at Ser 345 sites was used as a bio-marker of ATR-mediated signaling pathway. The phosphorylation of Rad17 at Ser 645, which is a substrate of ATR, was also examined. As shown in FIG. 6, JC099 and JC081 attenuated OLA-induced phosphorylation of Rad17 and CHK1 and in prostate cancer cells (FIG. 6A), breast cancer SUM149PT-BRCA1ᵣₑᵥ cells (FIG. 6B) and ovarian cancer UWB1.289+BRCA1 cells (FIG. 6C). Collectively, JC099 can directly bind to ATR and inhibit ATR activity as well as ATR-mediated signaling pathway.

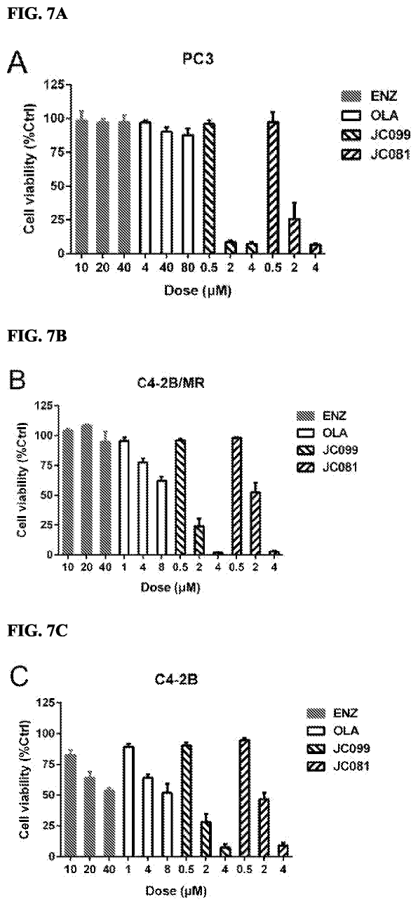

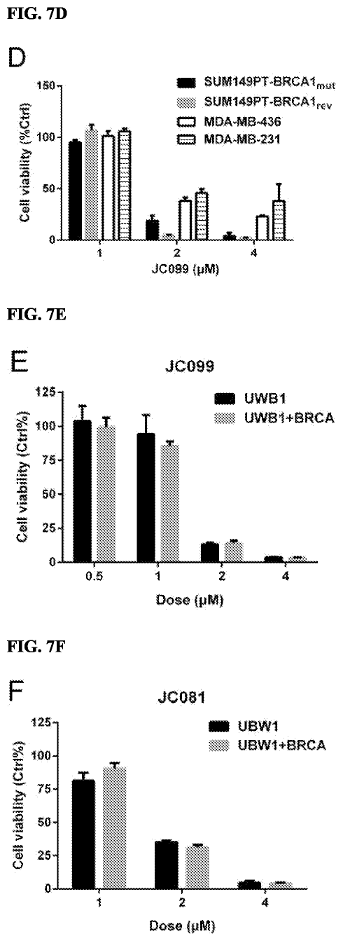

### Cell Killing Potency of JC081 and JC099

Whereas HR-proficient PC3 cells were insensitive to OLA (FIG. 7A), HR-mutated prostate cancer cells, such as C4-2B and C4-2B/MR (FIGS. 7B & 7C), exhibited pronounced sensitivity to OLA due to their inability to repair the DSBs. However, JC099 and JC081 were more cytotoxic in both HR-deficient and -proficient cells, including C4-2B, C4-2B/MR, and PC3 cells 48 hours after treatment, presumably due to combination of PARP-trapping and ATR inhibition. JC099 and JC081 (2 μM) resulted in more than 50% growth inhibition of all three prostate cancer cell lines in a CCK8 assay (FIGS. 7A-7C). JC099 also exerted strong growth inhibition in both BRCA1-mutunt, SUM149PT-BRCA1ₘᵤₜ and MDA-MB-436, and BRCA1-proficient SUM149PT-BRCA1ᵣₑᵥ and MDA-MB-231 cells. 2 μM of JC099 resulted in more than 60% growth inhibition in all four breast cancer cell lines in 48 hours (FIG. 7D). Similar results were also observed in ovarian cancer cells. Both UWB1 and UWB1+BRCA1 cells were sensitive to JC099 and JC081, which accounted for ~80% and ~70% growth inhibition at a dose of 2 μM, respectively (FIGS. 7E & 7F).

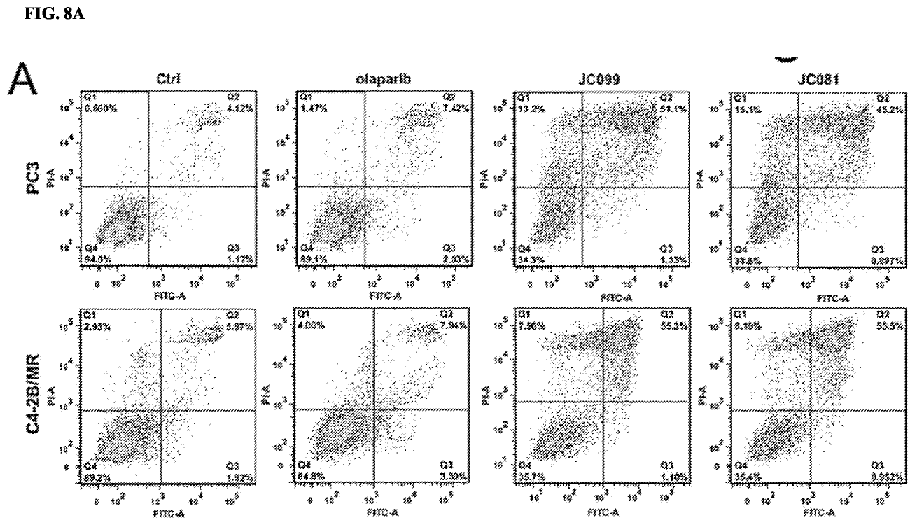

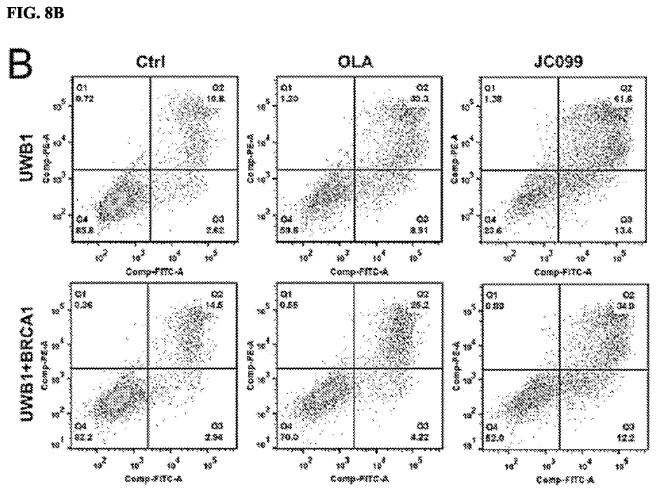

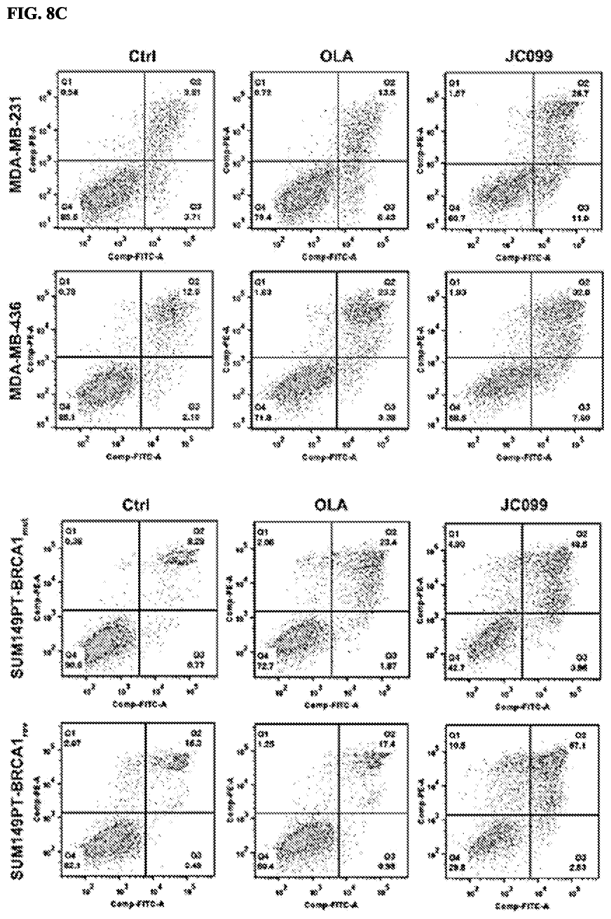

Further Annexin V staining showed that JC099 and JC081 induced cell apoptosis. As shown in FIG. 8A, OLA (25 μM) induced less than 8% apoptosis in prostate cancer PC3 and C₄-2B/MR cells, while JC099 and JC081 (4 μM) induced ~50% apoptosis. JC099 also resulted in higher apoptosis than OLA in ovarian and breast cancer cells. 4 μM of JC099 induced ~60% and −35% apoptosis in UWB1 and UWB1+BRCA1 cells, respectively (FIG. 8B). In breast cancer cells, ~30% apoptosis was observed in MDA-MB-436 and MDA-MB-231 cells exposed to 4 μM of JC099 for 48 h, while ~50% apoptosis was observed in SUM149PT-BRCA1ₘᵤₜ and SUM149PT-BRCA1ᵣₑᵥ cells (FIG. 8C). These results indicate that JC081 and JC099 can inhibit cancer cell proliferation and induce cell apoptosis irrespective of BRCA and HR status.

### JC081 and JC099 Overcome PARPi Resistance and Inhibit Breast Cancer Growth In Vivo

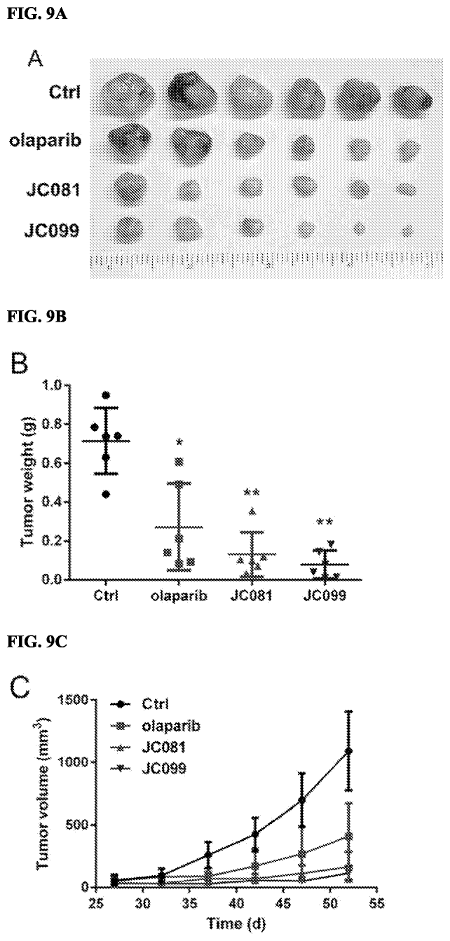

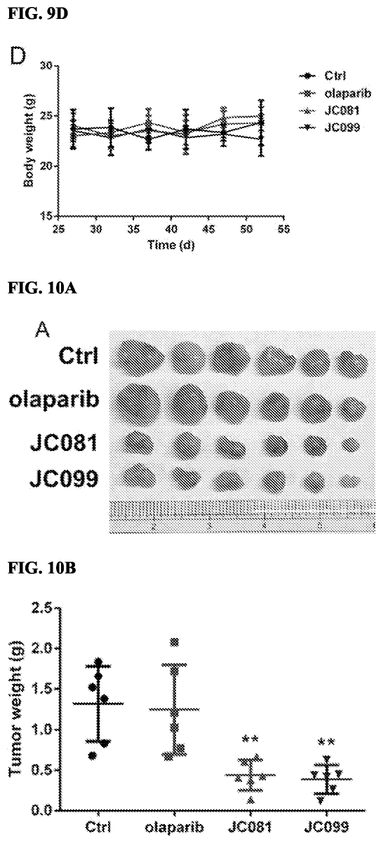

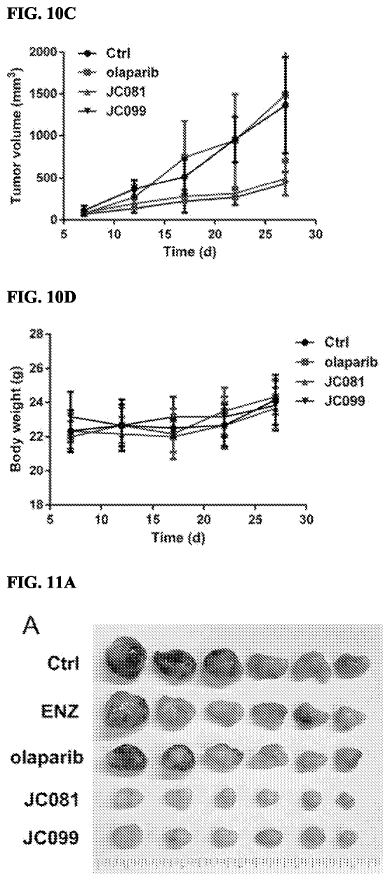

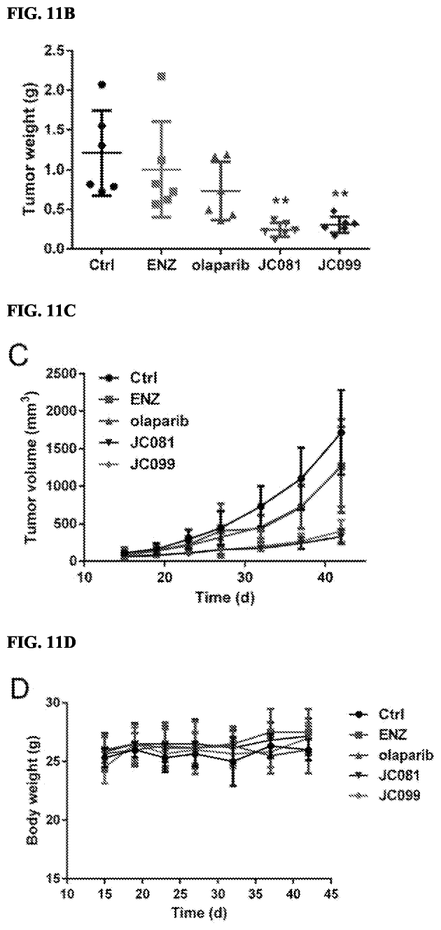

It was determined the antitumor effect of JC081 and JC099 in vivo by xenografts. Mice were subcutaneously implanted with MDA-MB-436 and MDA-MB-231 cells. When the tumors reached ~100 mm³, mice were intraperitoneally (i.p.) administered 10 mg/kg of JC081, JC099 or 60 mg/kg of OLA five times weekly. Equivalent solvent was used as control. The longest (L) and shortest (W) tumor axes were measured, and tumor volume (mm³) was calculated as L×W²/2. Compared to the vehicle controls, the JC099 and JC081 treatment decreased tumor weight by ~80%-85% in HR-deficient MDA-MB-436 xenografts while OLA resulted in ~60% decrease in tumor weight (FIGS. 9A & 9B). These results were also evidenced by tumor growth curves (FIG. 9C). In addition, the body weights of tumor-bearing mice were recorded twice a week to determine toxicity. Both JC099 and JC081 did not induce any decrease in body weights in mice (FIG. 9D). MDA-MB-231 cells have wild-type BRCA1 and are HR-proficient which are resistant to PARPi. The JC081 and JC099 treatment significantly decreased tumor weights by 70% in MDA-MB-231 xenografts while OLA did not significantly affect tumor weights (FIGS. 10A & 10B). These results were also evidenced by tumor growth curves (FIG. 10C). Both JC099 and JC081 did not affect body weights of MDA-MB-231 tumor-bearing mice (FIG. 10D). These results suggest that JC081 and JC099 can overcome PARPi resistance.

### JC081 and JC099 Overcome Enzalutamide (ENZ) Resistance and Inhibit Prostate Cancer Growth In Vivo

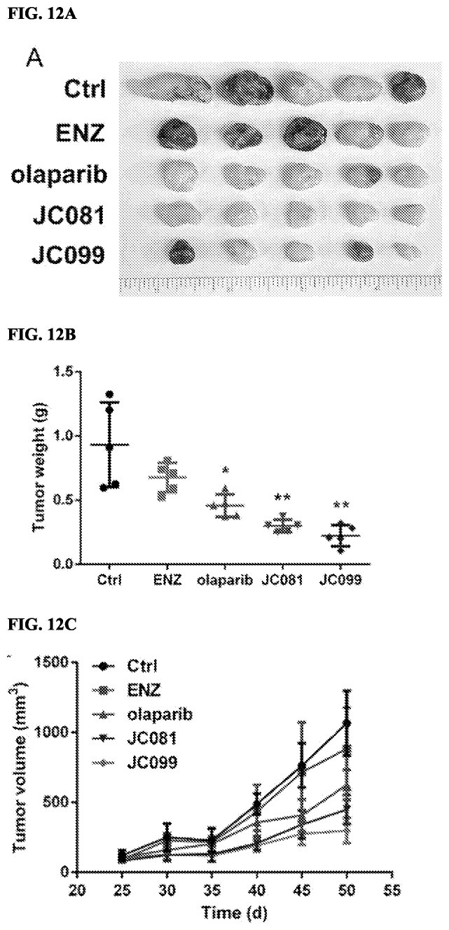

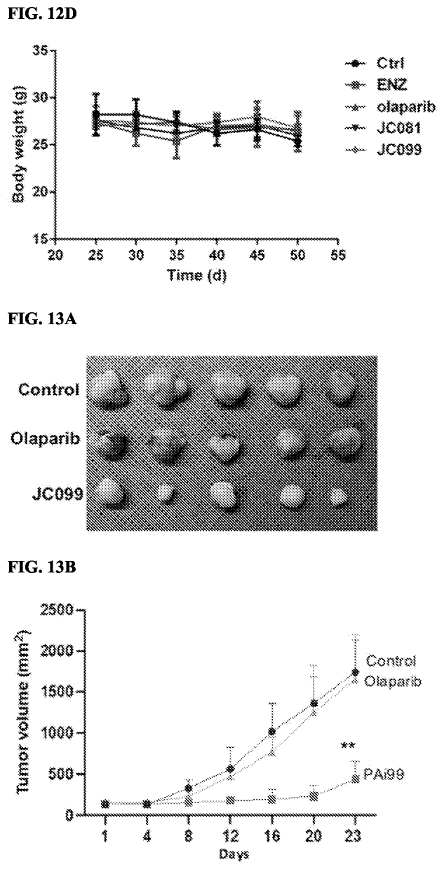

PARPi has been used to treat prostate cancer. Enzalutamide (ENZ), also known as Xtandi, is a nonsteroidal antiandrogen for treating metastatic castration-resistant prostate cancer. While both prostate cancer cell lines PC3 and C4-2B/MR are ENZ-resistant, PC3 is HR-proficient and C4-2B/MR is HR-deficient. It was examined whether JC099 and JC081 can overcome ENZ resistance in these cells. The JC081 and JC099 treatment significantly inhibited tumor growth of PC3 in vivo compared with ENZ and OLA treatment (FIGS. 11A-11C). Similarly, the JC081 and JC099 also significantly inhibited tumor growth of C4-2B/MR cells in vivo compared with ENZ and OLA treatment (FIGS. 12A-12C). The JC081 and JC099 treatment did not affect mouse body weights (FIG. 11D and FIG. 12D). Taken together, these results demonstrate that JC081 and JC099 can overcome ENZ resistance in prostate cancers.

### JC099 Overcomes Cisplatin Resistance in Head and Neck Squamous Cell Carcinoma (HNSCC) and Non-Small Cell Lung Cancer (NSCLC)

Cisplatin is a DNA damaging agent which is widely used for first-line therapy, including testicular cancer, ovarian cancer, cervical cancer, breast cancer, bladder cancer, head and neck cancer, esophageal cancer, lung cancer, mesothelioma, brain tumors and neuroblastoma. Unfortunately, more than 50% of patients develop resistance after treatment. It is known that cisplatin treatment can induce ATR activities. Therefore, it was examined whether JC099 could inhibit tumor growth of the cisplatin-resistant HNSCC cell line SCC1R in vivo. JC099 treatment, but not OLA, significantly inhibited tumor growth of SCC1R cells in vivo (FIGS. 13A & 13B). H1703R is a cisplatin-resistant NSCLC cell line. Since JC099 has a dual function, the efficacy of JC099 was compared with the combination of PARPi and ATRi in H1703R xenografts. It was also found that JC099 inhibited tumor growth of H1703R cells more potently than the combination of OLA and VE-822, a well-known ATRi (FIGS. 13C & 13D). Taken together, these results suggest that JC099 could overcome cisplatin resistance and are more efficient in the inhibition of tumor growth than the combination of individual PARPi and VE-822.

### Optimization of JC099 Structure

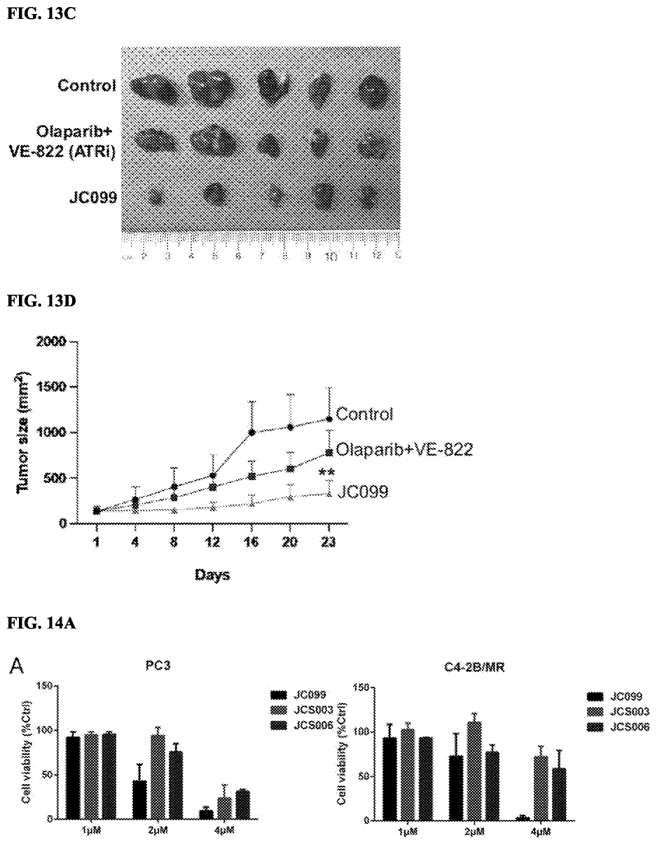

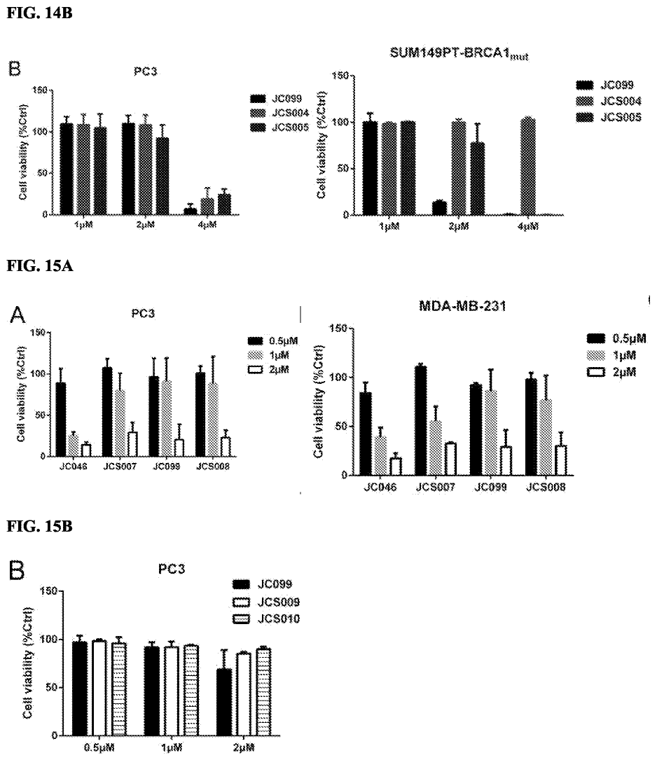

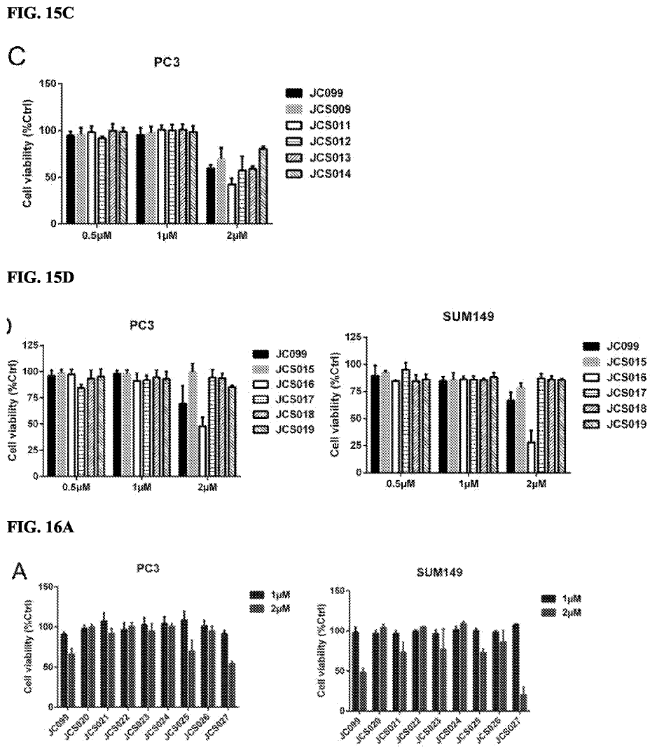

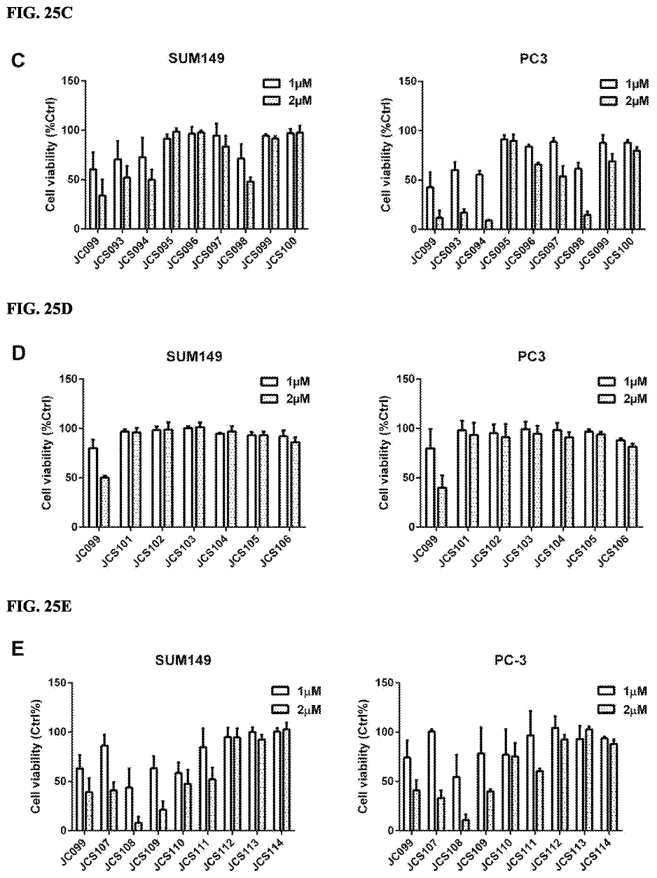

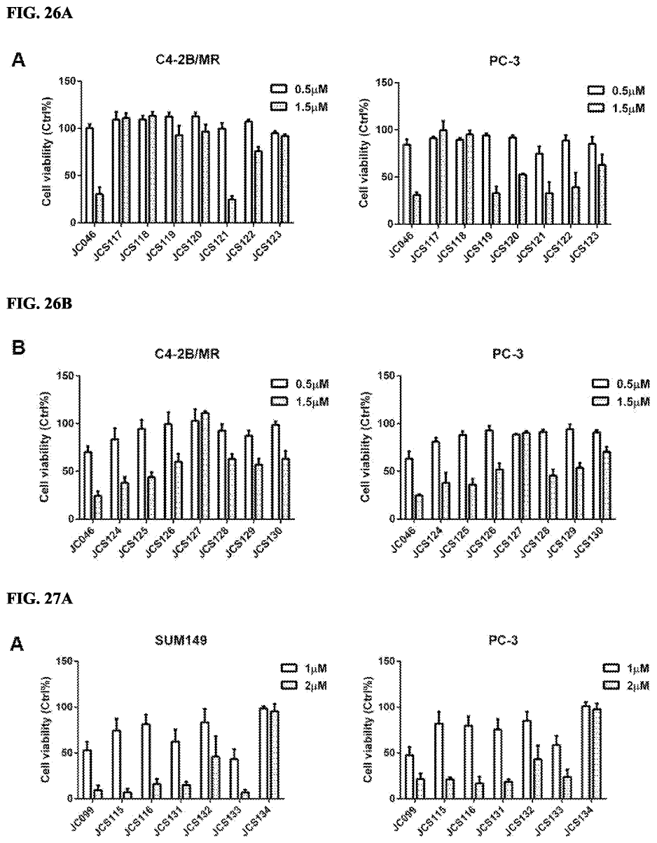

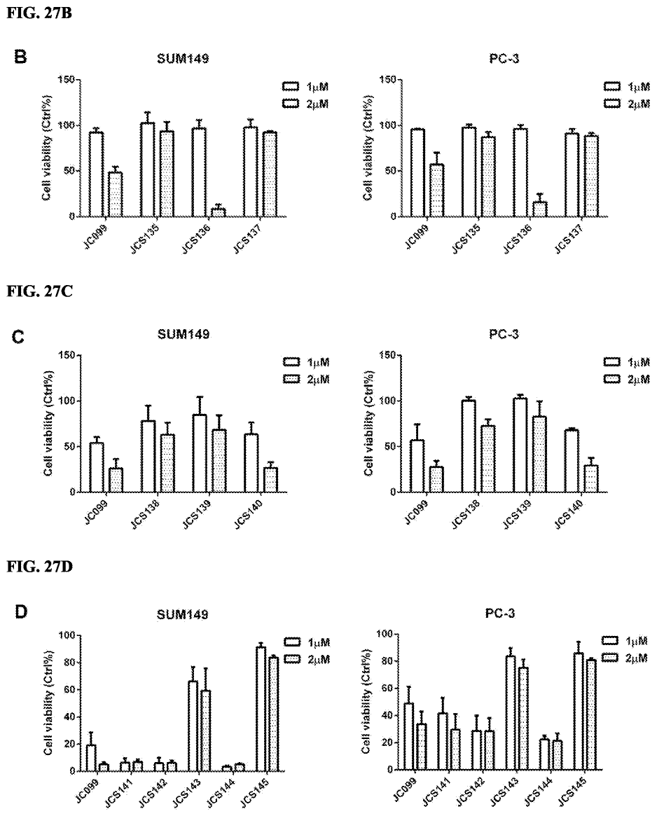

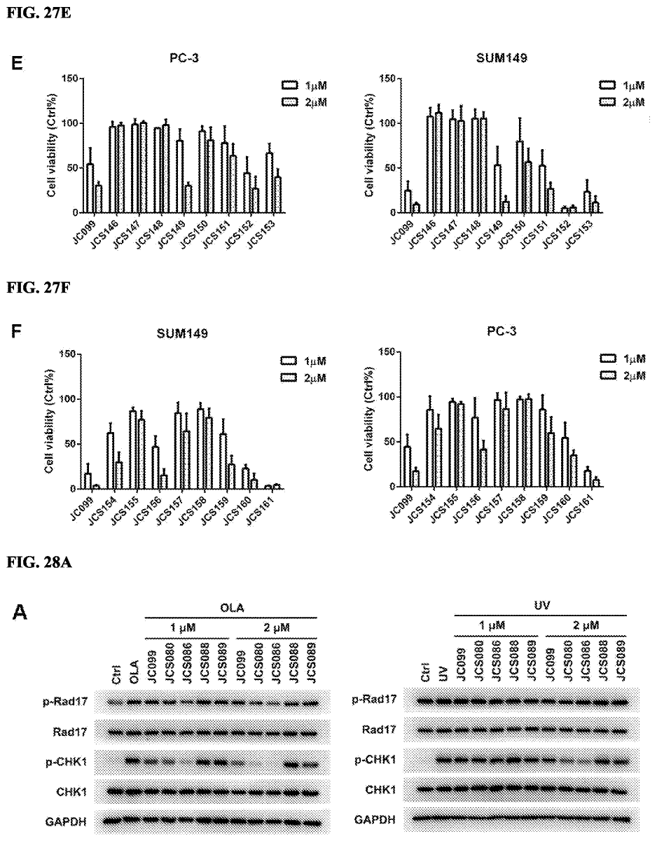

Structure optimization represents a critical step towards identifying candidate compounds for preclinical and clinical development and contributes to structure-function analysis. 69 analogs of JC099, JCS003-JCS071 were developed and screened as potential candidates by combining cell killing, PARP1 trapping, and ATR inhibition potency. Cell killing potency was detected by a CCK8 assay in prostate and breast cancer cells. As shown in FIGS. 14-16, JCSO11/16/27/41/43/46/69/71 had higher cell killing potency than JC099 in PC3 and SUM149PT-BRCA1ₘᵤₜ cells. Additionally, 65 analogs of JC099 were further developed, including JCS085-116 and JCS131-161, and screened potential candidates by combining cell killing, PARP1 trapping, and ATR inhibition potency. Cell killing potency in prostate and breast cancer cells was detected by a CCK8 assay. As shown in FIG. 25-27, JCS086/90/91/108/136/140/141/142/144/152/161 had higher cell killing potency than JC099 in PC3 and SUM149PT-BRCA1ₘᵤₜ cells, while JCS092/93/94/98/107/109/115/116/131/133/149/153/160 exhibited similar cell killing potency relative to JC099. Some other analogs, including JCS110/111/132/138/139/143/154/156, also had moderate potency of cell killing, whereas less than JC099.

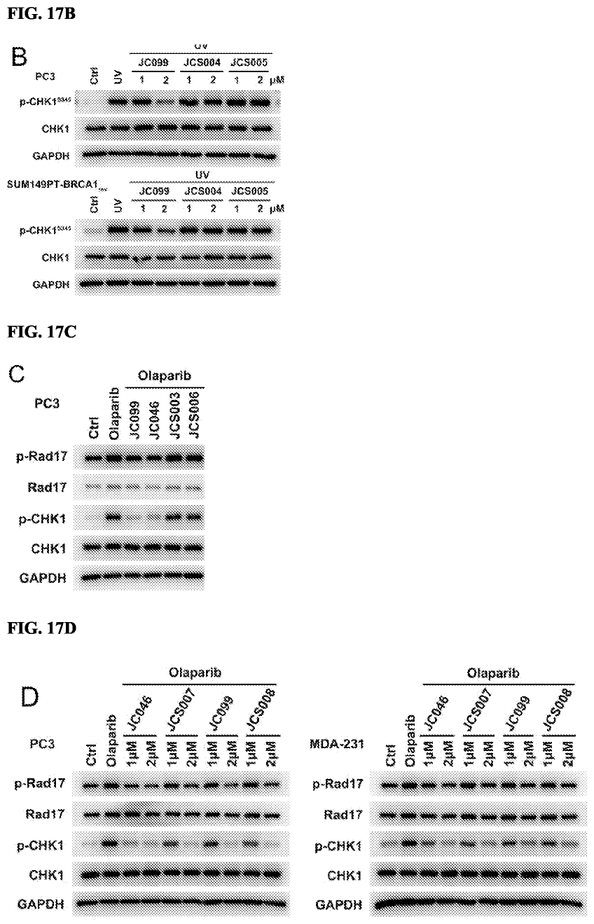

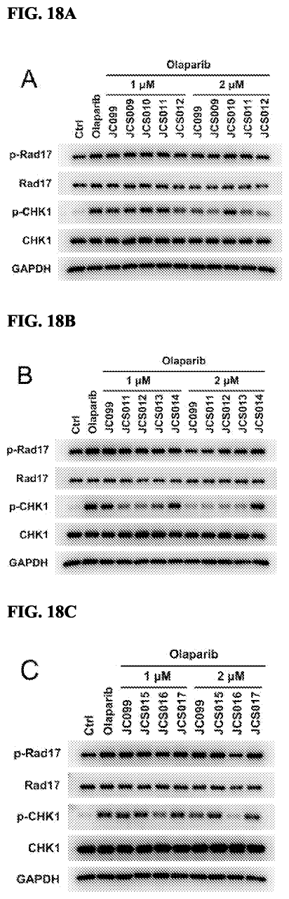

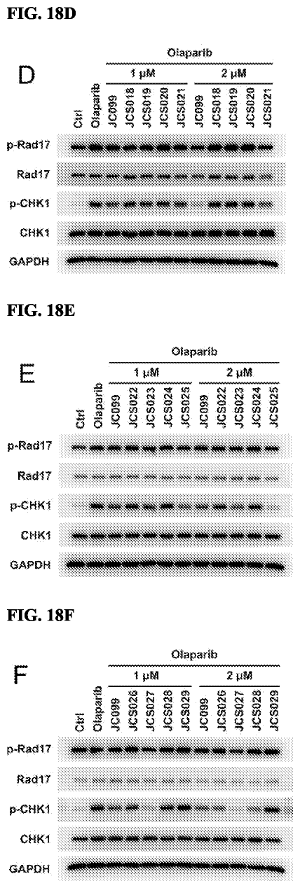

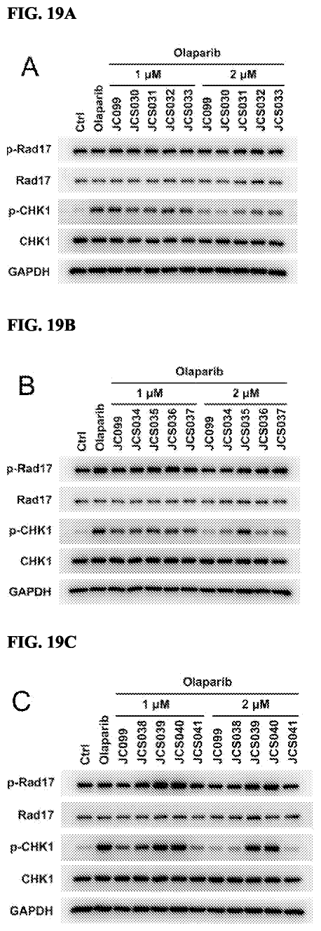

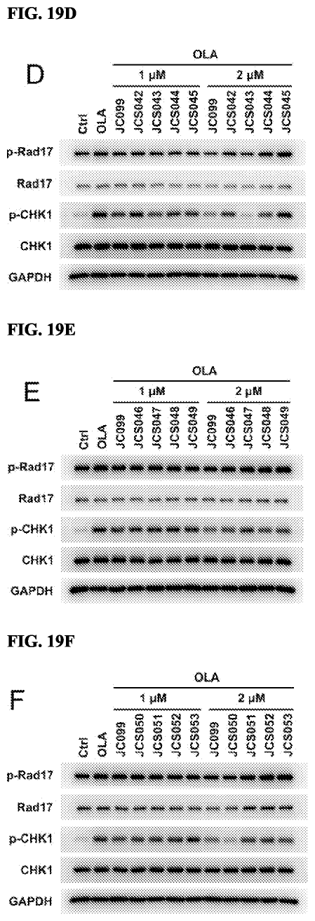

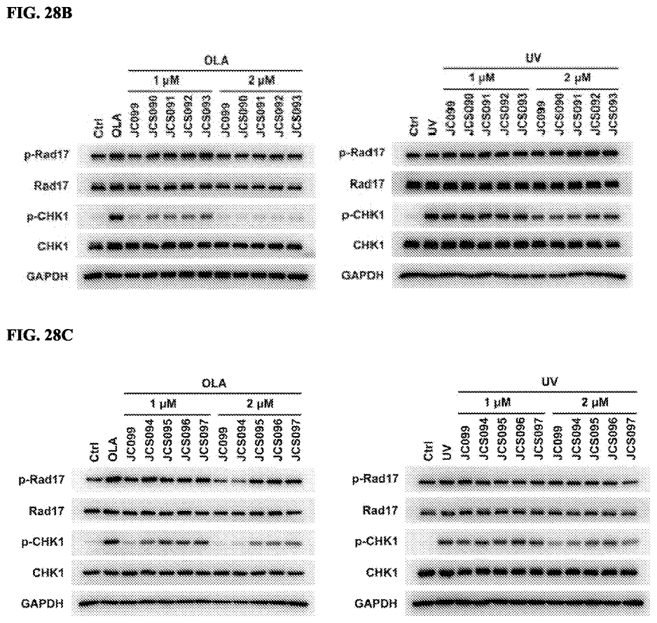

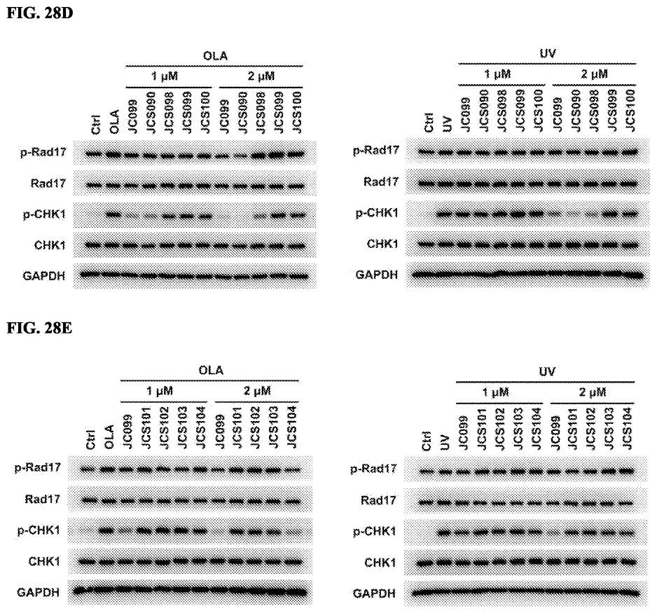

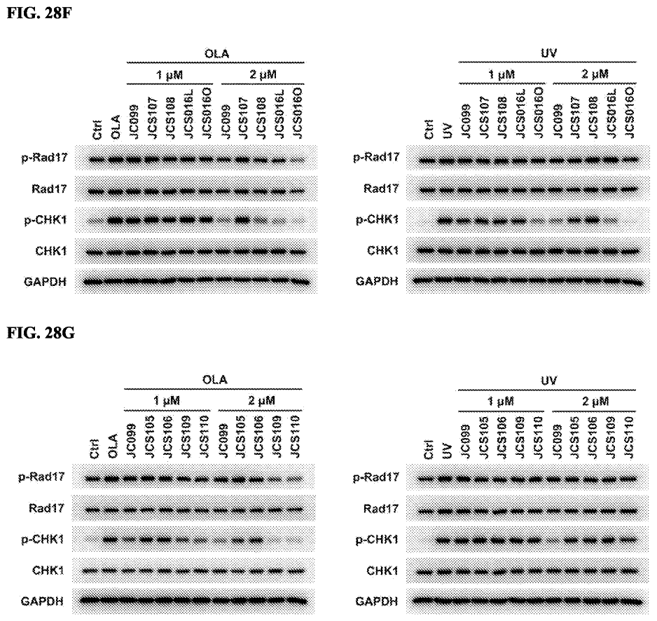

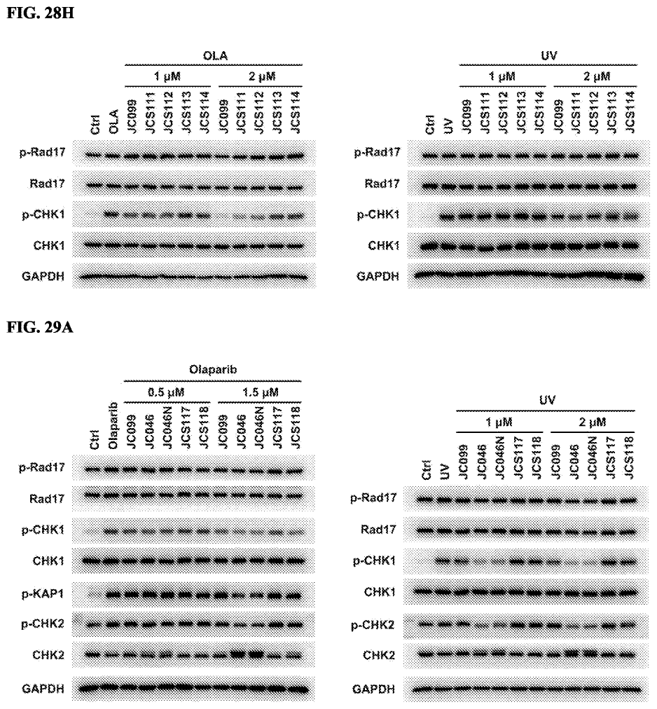

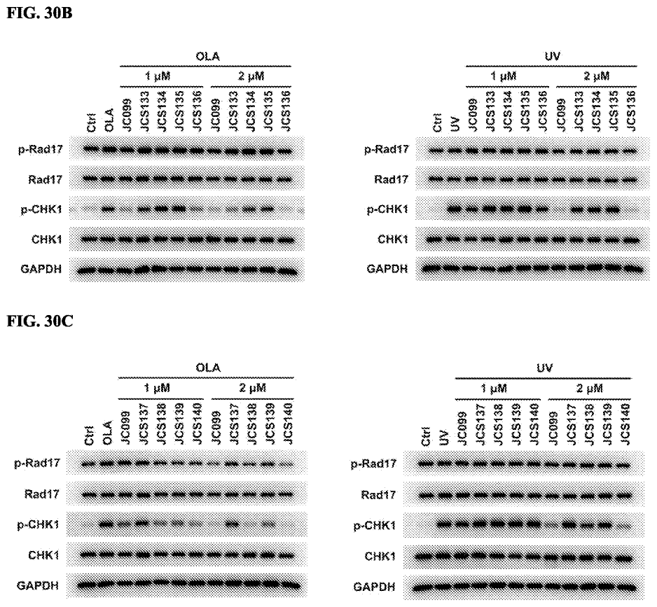

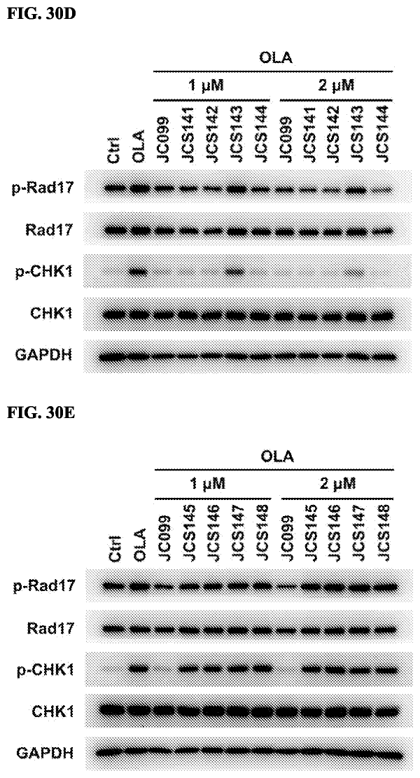

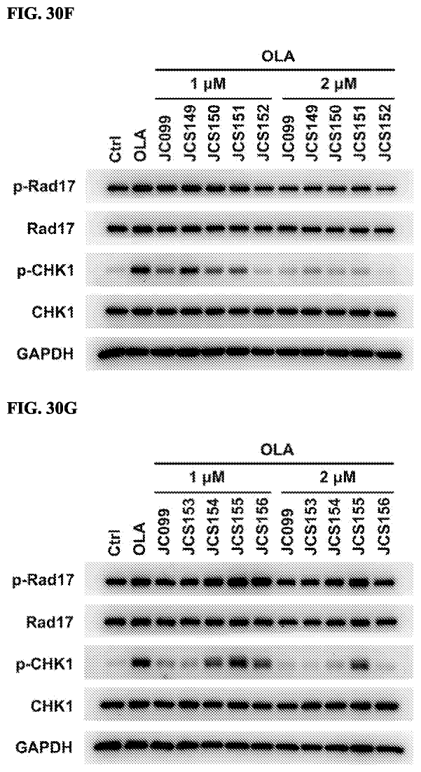

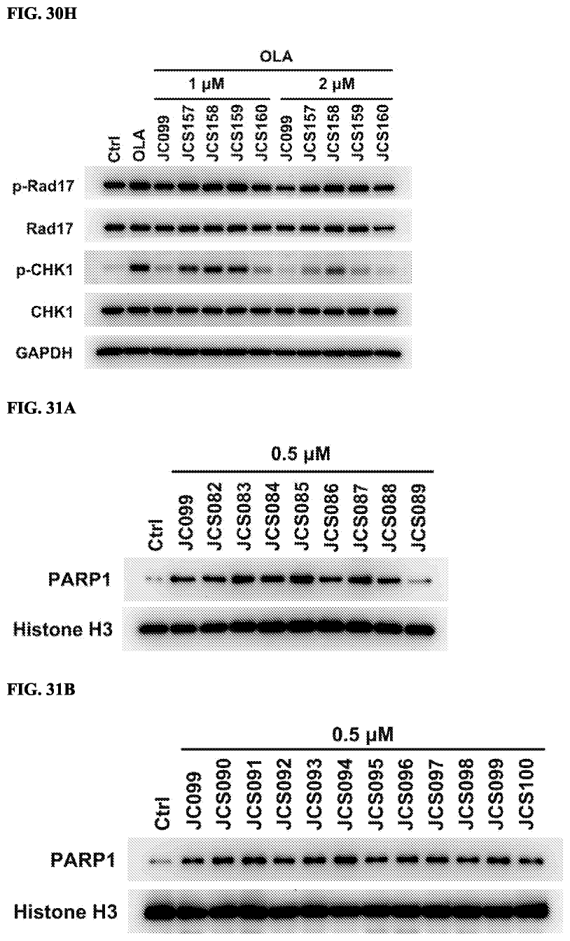

ATR inhibition potency of JC099 analogs was evaluated by decreasing OLA- or UV-induced phosphorylation of Rad17 at Ser 645 and CHK1 at Ser 345 sites. As shown in FIGS. 17-20, the phosphorylation of Rad17 and CHK1 decreased more dramatically after the treatment with JCS012/16/25/27/41/43/50/63/66/69 than JC099, indicating greater ATR inhibition potential of JCS012/16/25/27/41/43/50/63/66/69 than JC099. As shown in FIGS. 28-30, the phosphorylation of Rad17 and CHK1 decreased more dramatically after the treatment with JCS086/90/109/110/136/140/141/142/144/152/153/160 than JC099, indicating greater ATR inhibition potential of JCS086/90/109/110/136/140/141/142/144/152/153/160 than JC099. Other analogs, including JCS0091/92/94/111/133/138/150/151/154/156, decreased OLA-induced phosphorylation of Rad17 and CHK1, while their potencies were close to JC099. Compared to JC099, JCSO93/95/97/98/104/108/112/115/116/132/139/143/149/157/159 had less ATR inhibition potencies, albeit they also reduced OLA- or U-induced phosphorylation of Rad17 and CHK1.

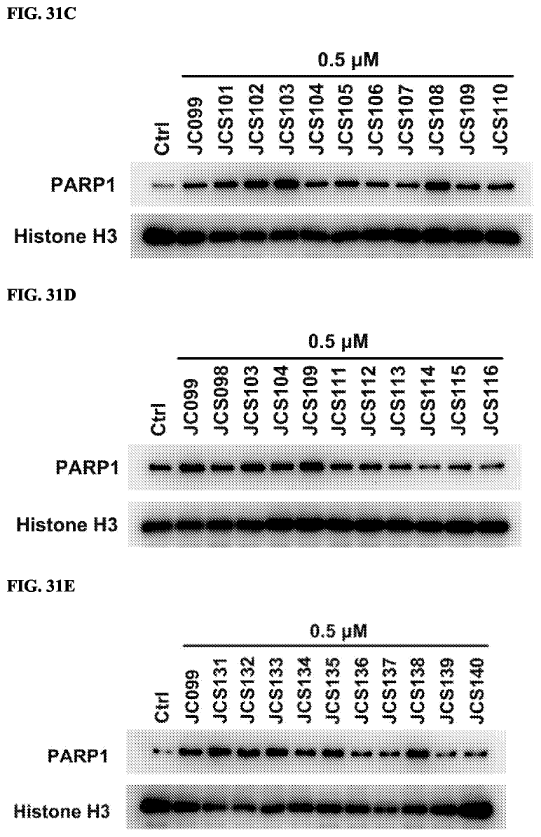

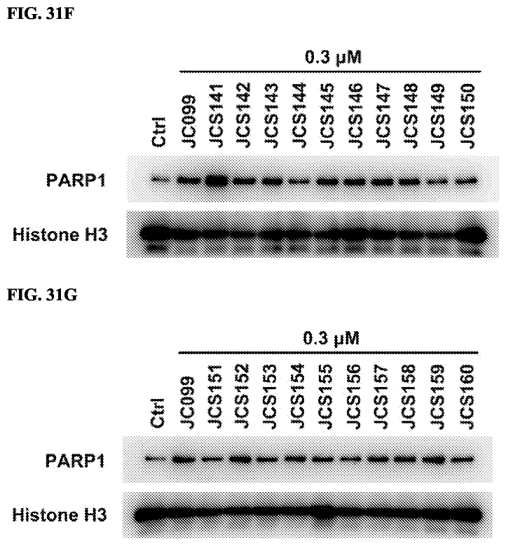

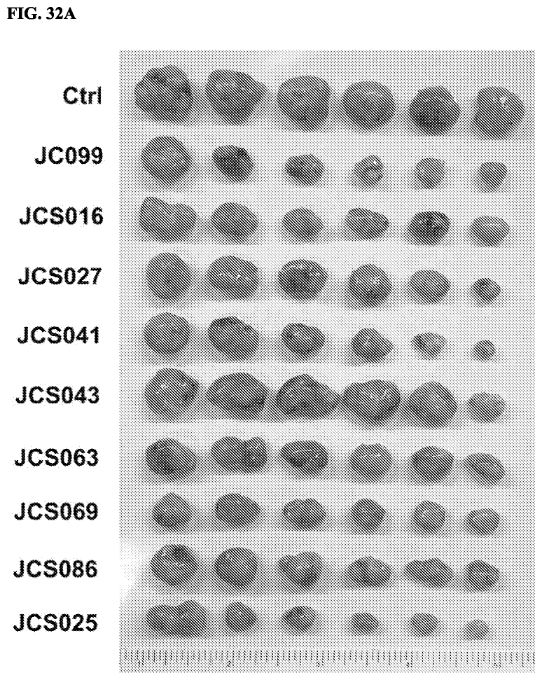

The chromatin-bound proteins were isolated from cells upon the treatment of JC099 or its analogs to analyze PARP1 proteins trapped on DNA lesions. As shown in FIG. 31, an increase of PARP1 in the chromatin-bound fraction was observed in PC-3 cells with treatment of JCS085/87/90/91/94/99/102/103/108/109/131/132/133/135/138/141/159 relative to JC099, indicating higher potency in PARP1-trapping of JCS085/87/90/91/94/99/102/103/108/109/131/132/133/135/138/141/159 than JC099. JCS086/88/92/93/95/96/97/98/100/101/104/105/106/107/109/110/134/136/142/143/145~148/152/157/158 has similar potency in PARP1 trapping compared to JC099. JCS089/111-116/137/139/140/144/149/150/151/153-156/160 also increased the accumulation of PARP1 in the chromatin-bound fraction, but their potencies were less than JC099.

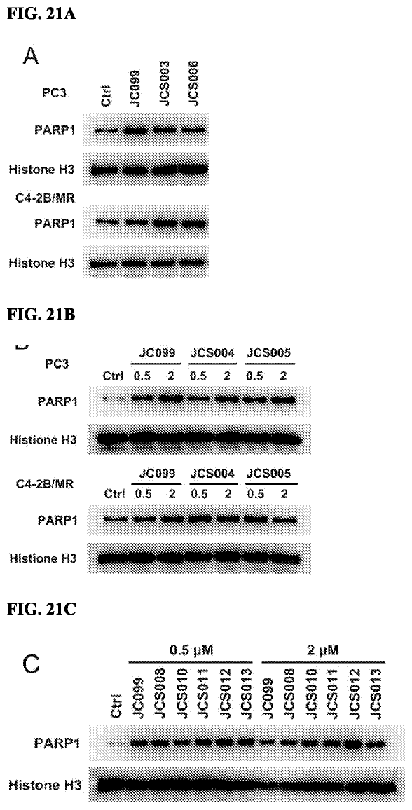

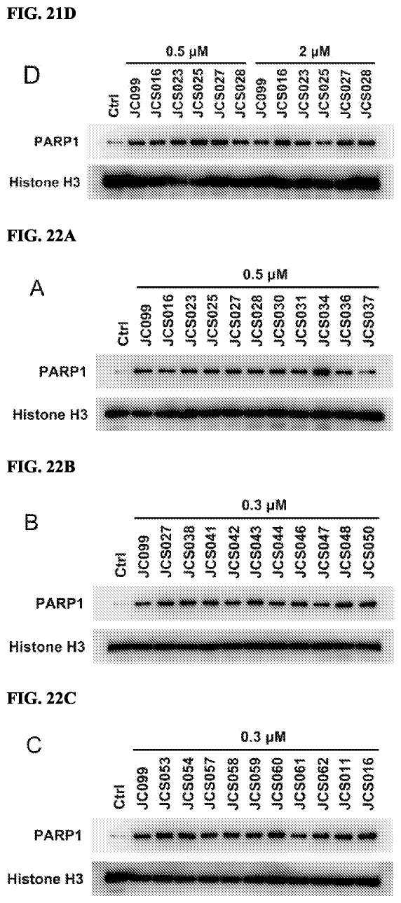

For PARP1 trapping FIGS. 21 & 22, JCS008/11/12/13/25/34/43/48/50/53/54/60/65/70 increased the accumulation of PARP1 in the chromatin-bound fraction than JC099 in PC3 cells, indicating higher potency in PARP1-trapping of JCS008/11/12/13/25/34/43/48/50/53/54/60/65/70 relative to JC099.

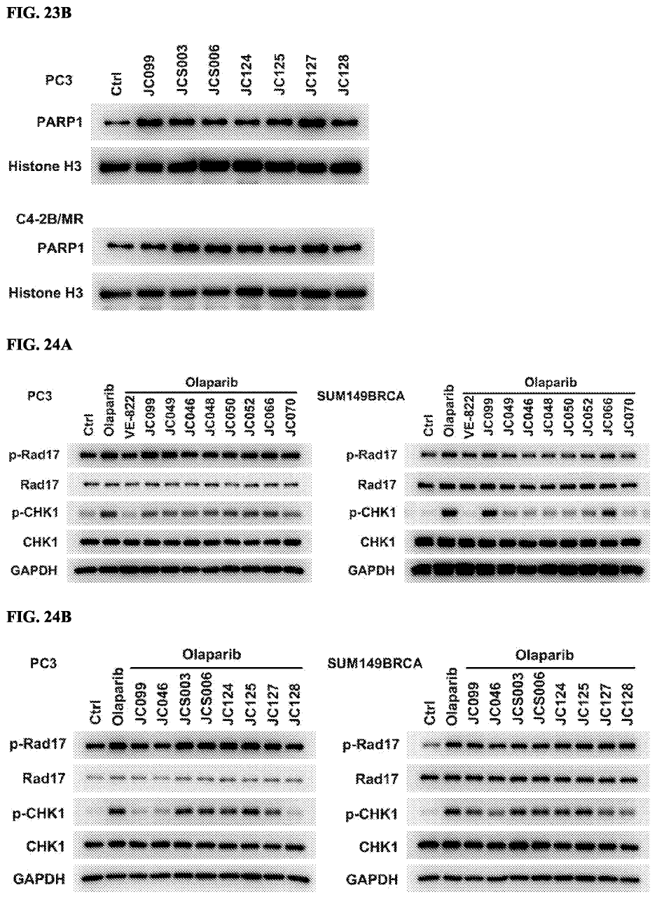

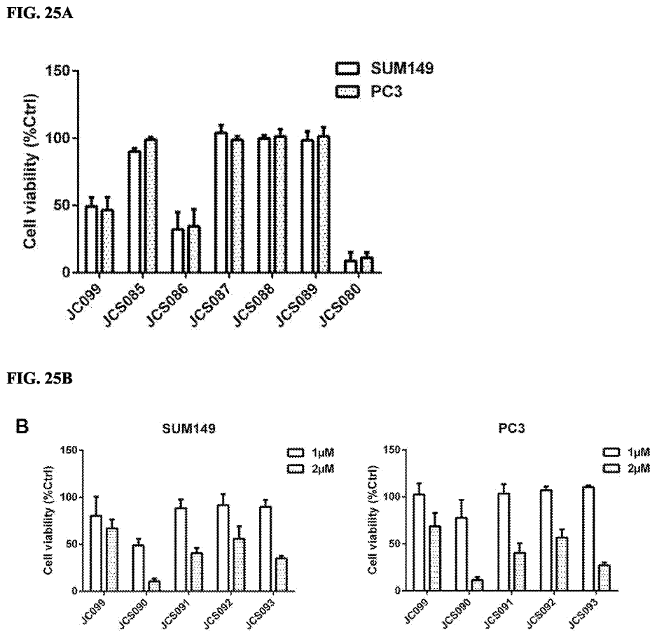

JC113, JC117, and JC127 exhibited higher potency than also JC099 (FIG. 23). For ATR inhibition activity, JC046, JC048, JC049, JC050, JC070, and JC128 had higher potency than JC099.

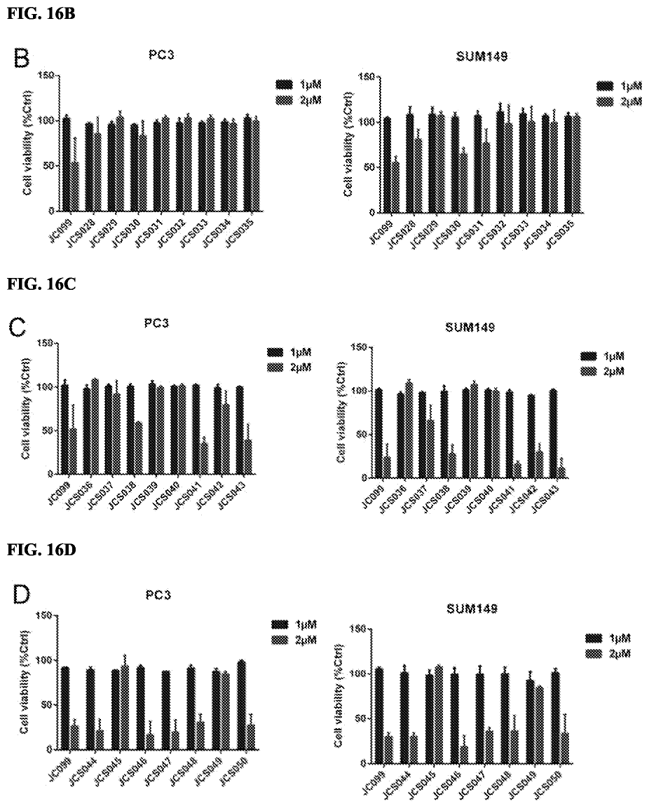

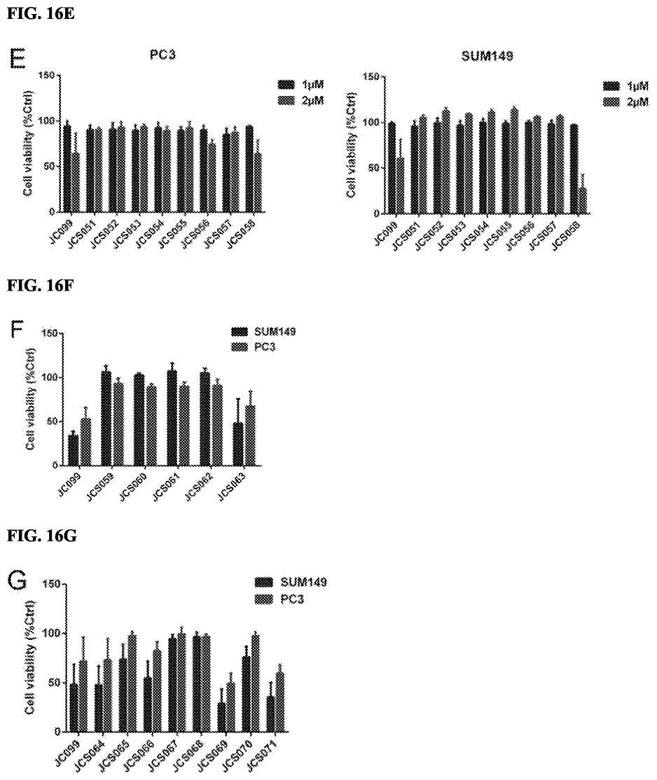

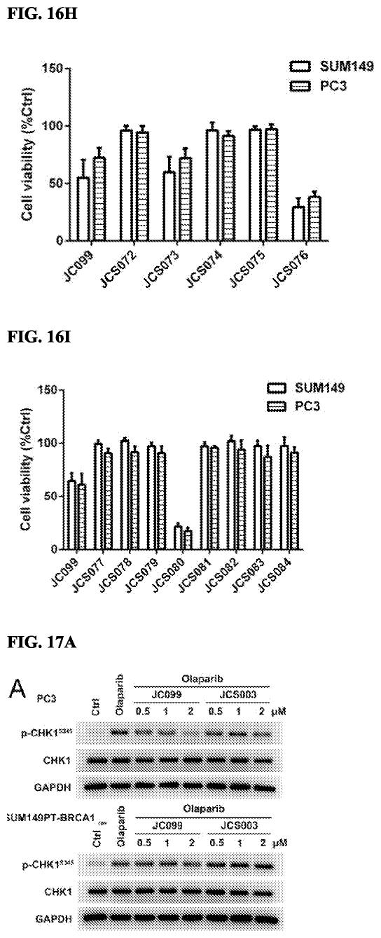

JCS72-87 also showed the ability to trap PARP1 and inhibit ATR. The cell killing potency was detected by a CCK8 assay in prostate and breast cancer cells. As shown in FIGS. 16H&I, JCSO76 and JCS080 had higher cell killing potency than JC099 in both PC3 and SUM149PT-BRCA1ₘᵤₜ cells. JCSO73 had close cell killing potency relative to JC099.

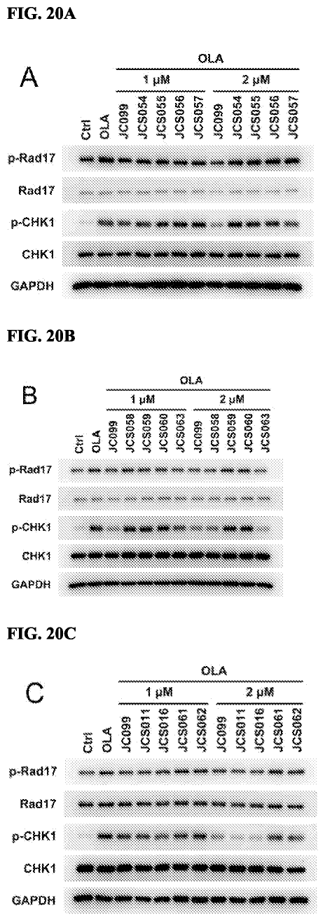

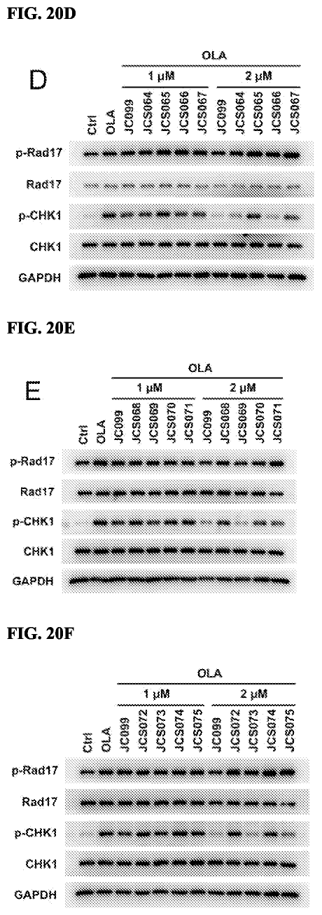

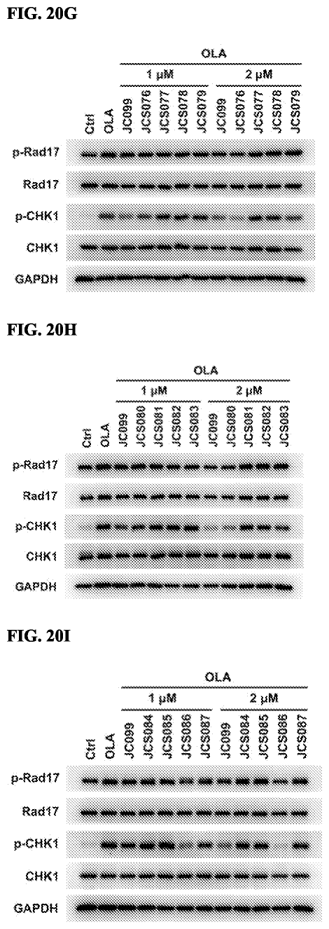

ATR inhibition potency of JC099 analogs was evaluated by decreasing OLA induced phosphorylation of Rad17 at Ser 645 and CHK1 at Ser 345 sites. As shown in FIG. 20F-I, the phosphorylation of Rad17 and CHK1 decreased more dramatically after the treatment with JCSO73/76/80/86 than JC099, indicating greater ATR inhibition potential of JCSO73/76/80/86 than JC099. JCSO75 also decreased the phosphorylation of Rad17 and CHK1.

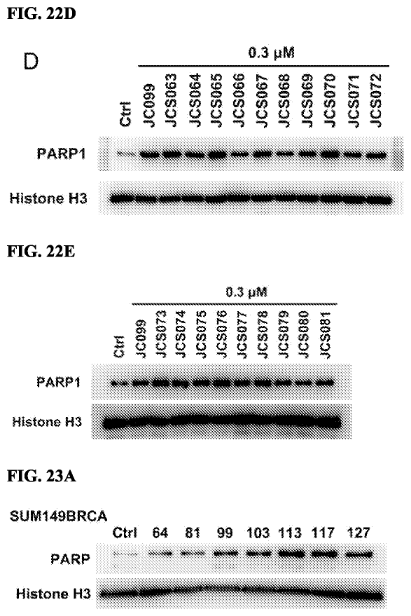

For PARP1 trapping (FIG. 22E), JCSO73/74/75/76/77/78 increased the accumulation of PARP1 in the chromatin-bound fraction than JC099 in PC3 cells.

To evaluate the anti-tumor activity of JC099 analogs in vivo, four xenografts were used, including two breast cancer PDX (Patient Derived Xenografts), one breast cancer CDX (Cell Derived Xenografts), and one castration-resistant prostate cancer PDX. Tumor tissues or cells were subcutaneously implanted in CB-17/SCID mice. When the tumors reached ~100 mm³, mice were administered orally (i.g.) JC099 or its analogs five times weekly. An equivalent solvent was used as a control. The longest (L) and shortest (W) tumor axes were measured, and tumor volume (mm³) was calculated as L×W²/2. In addition, the body weights of tumor-bearing mice were recorded twice a week to determine toxicity.

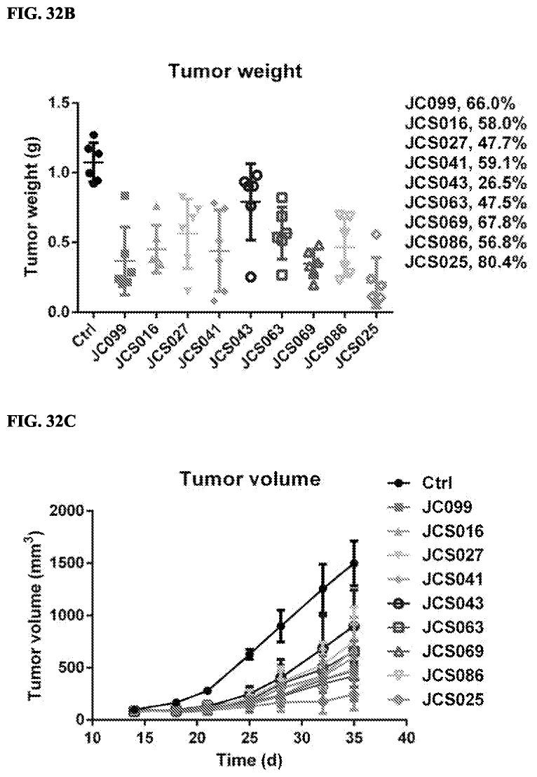

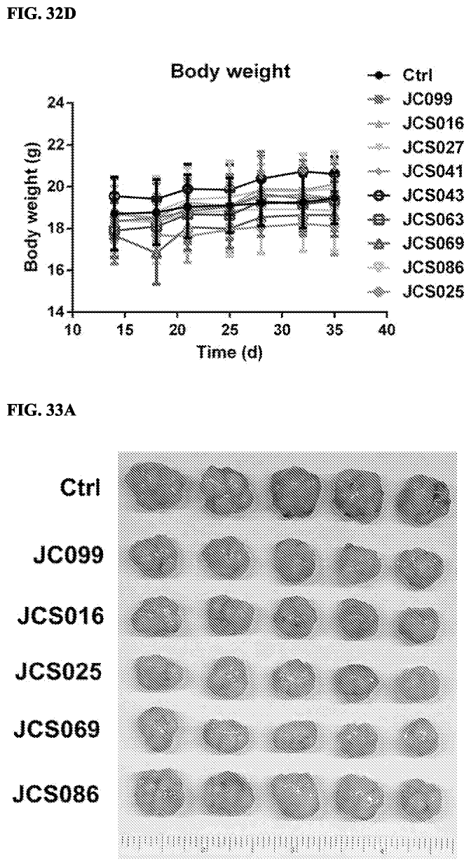

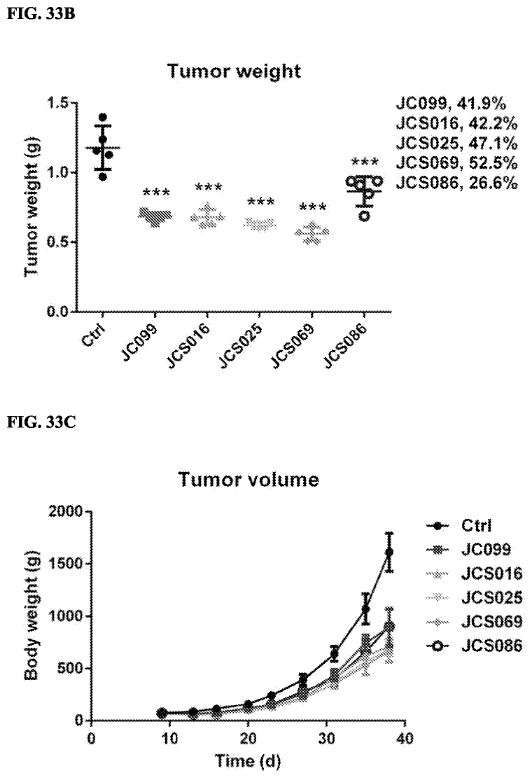

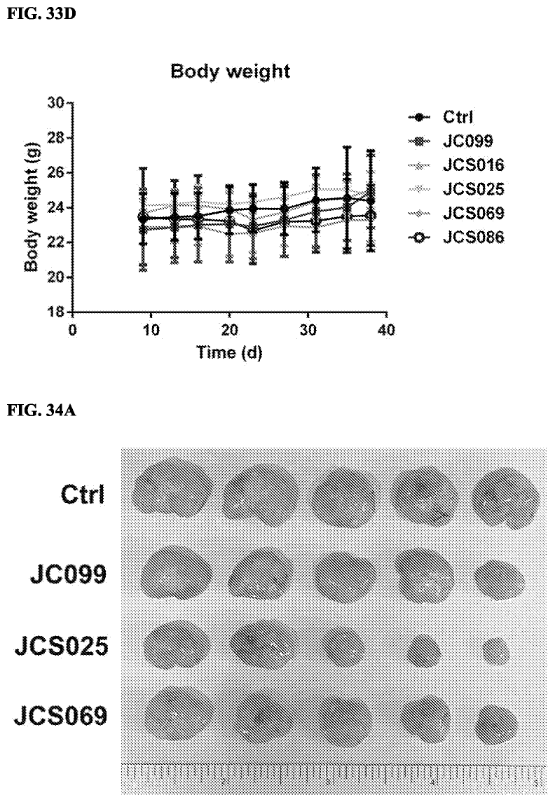

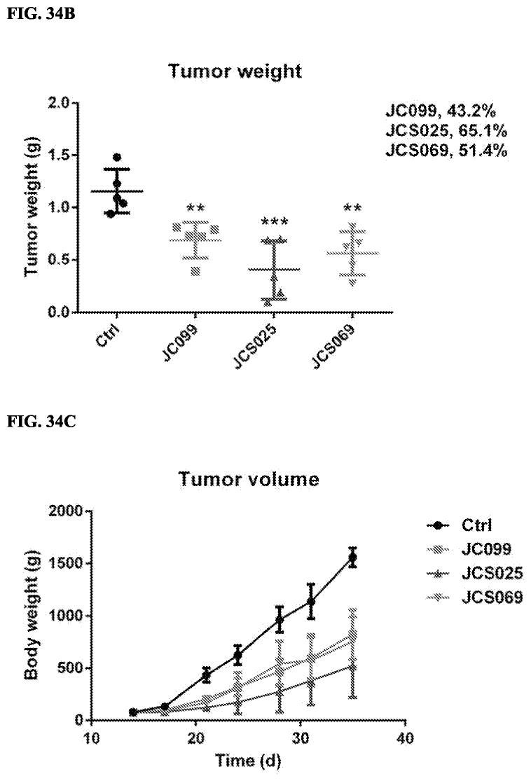

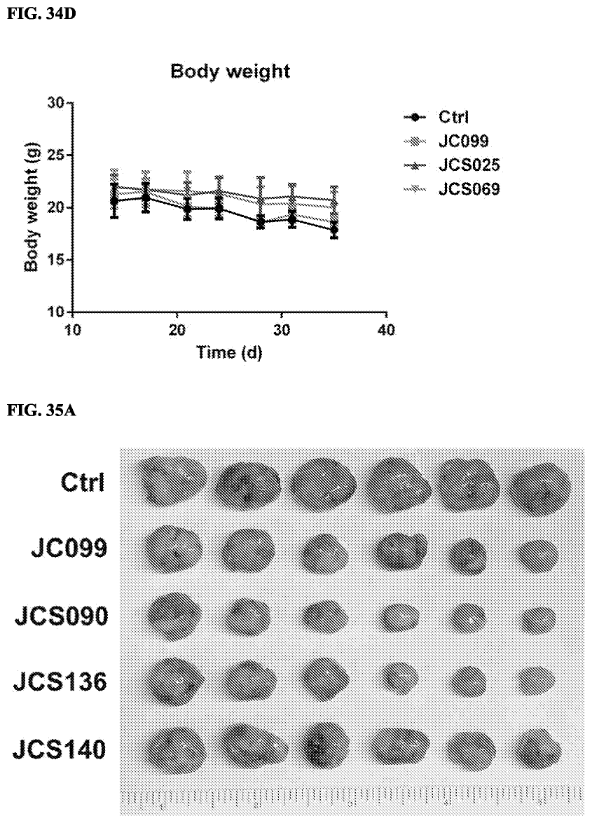

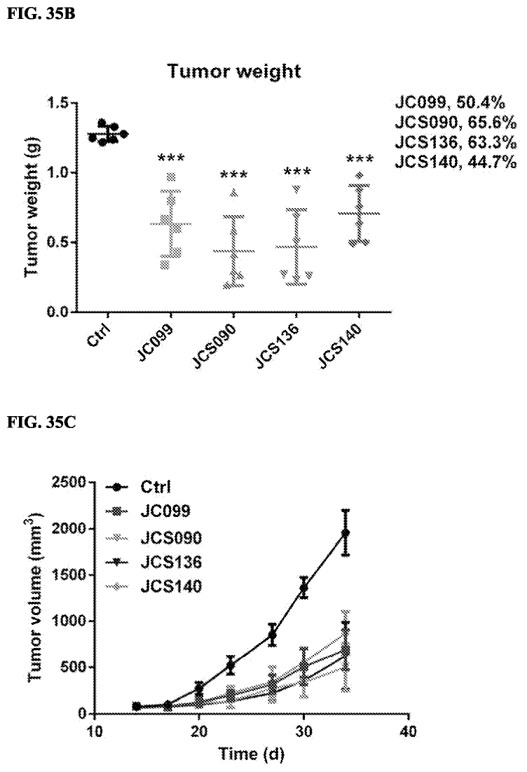

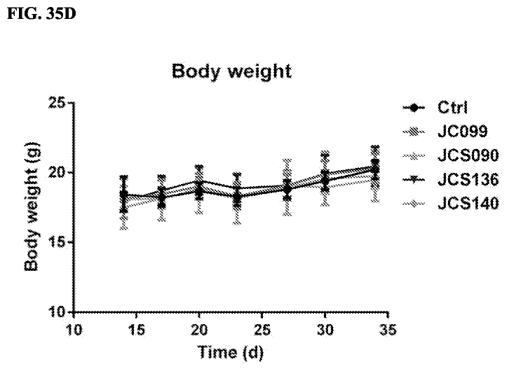

FIG. 32A-32D shows the results obtained in a BRCA2-mutant breast cancer PDX, which is sensitive to PARP inhibitors. Compared to the vehicle controls, JCS016/25/27/41/43/63/69/86 treatment suppressed tumor growth (FIGS. 32A & 32C) and decreased tumor weight by ~26-80% (FIG. 32B). Among them, JCSO25 and JCS069 exhibited higher activities than JC099. JCS016, JCSO41, and JCS086 also decreased tumor weight dramatically, but their potencies were slightly less than JC099. None of the analogs affected the body weights of tumor-bearing mice (FIG. 32D), indicating no obvious toxicity was observed.

FIG. 33A-33D shows the results obtained in the breast cancer SUM149PT-BRCA1ᵣₑᵥ cells derived xenografts, which are resistant to PARP inhibitors due to deleterious BRCA1 mutation is reverted to form a functional BRCA1. Compared to the vehicle controls, tumor growth was suppressed by the treatment of JCS016/25/69/86 (FIGS. 33A & 33C). JCSO25 and JCS069 decreased tumor weights by 47.1% and 52.5%, respectively, which is higher than JC099 (FIG. 33B). The activity of JCS016 is close to JC099. The activity of JCS086 is less than JC099. None of the analogs affected the body weights of tumor-bearing mice (FIG. 33D), indicating no obvious toxicity was observed.

FIG. 34A-34D shows the results obtained in a castration-resistant prostate cancer PDX, which has acquired resistance to Enzalutamide. Compared to the vehicle controls, tumor growth was significantly suppressed by the treatment of JCSO25 and JCS069 (FIGS. 34A & 34C). JCSO25 and JCS069 decreased tumor weights by 65.1% and 51.4%, respectively, which is higher than JC099 (FIG. 34B), indicating higher activity than JC099. None of the analogs affected the body weights of tumor-bearing mice (FIG. 34D), indicating no obvious toxicity was observed.

FIG. 35A-35D shows the results obtained in a breast cancer PDX, which has acquired resistance to PARP inhibitors. Compared to the vehicle controls, tumor growth was significantly suppressed by the treatment of JCS090, JCS136, and JCS140 (FIGS. 35A & 35C). JCS090 and JCS136 decreased tumor weights by ~65%, while JC099 decreased tumor weights by ~50% (FIG. 35B). The data shows that JCS090 and JCS136 exhibited higher activities than JC099. The activity of JCS140 is slightly less than JC099. None of the analogs affected the body weights of tumor-bearing mice (FIG. 35D), indicating no obvious toxicity was observed.

### Optimization of JC046 Structure

JC046 is a specific inhibitor of ATR, so 14 analogs of JC046 were derived to optimize its structure. Potential candidates were screened by combining cell killing and ATR inhibition potency.

Cell killing potency was detected by a CCK8 assay in prostate cancer cell lines, PC-3 and C4-2B/MR that is acquired resistance to Enzalutamide. As shown in FIG. 26, JCS121/124/125 had close cell killing potency relative to JC046, while JCS119/120/123/126/128/129/130 had moderate cell killing potency which was less than JC046.

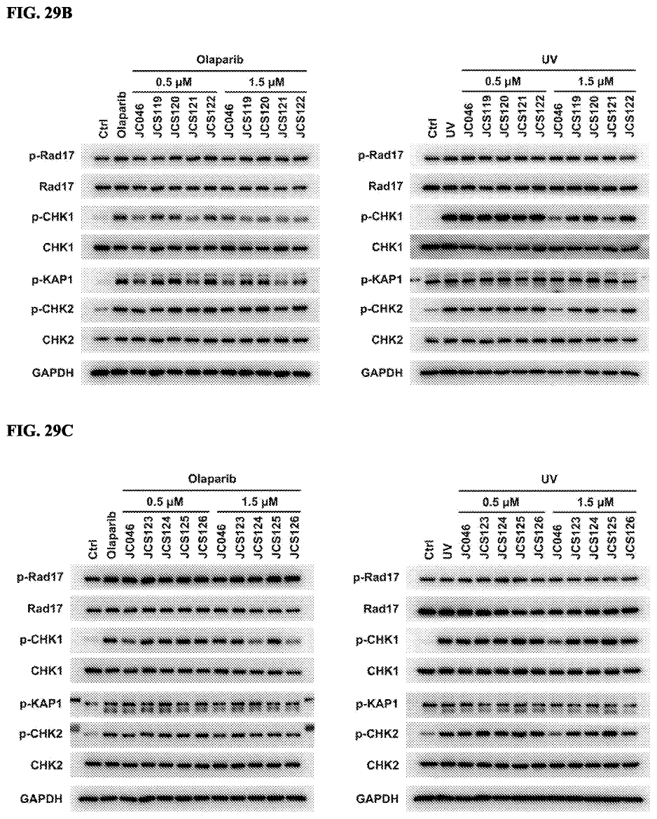

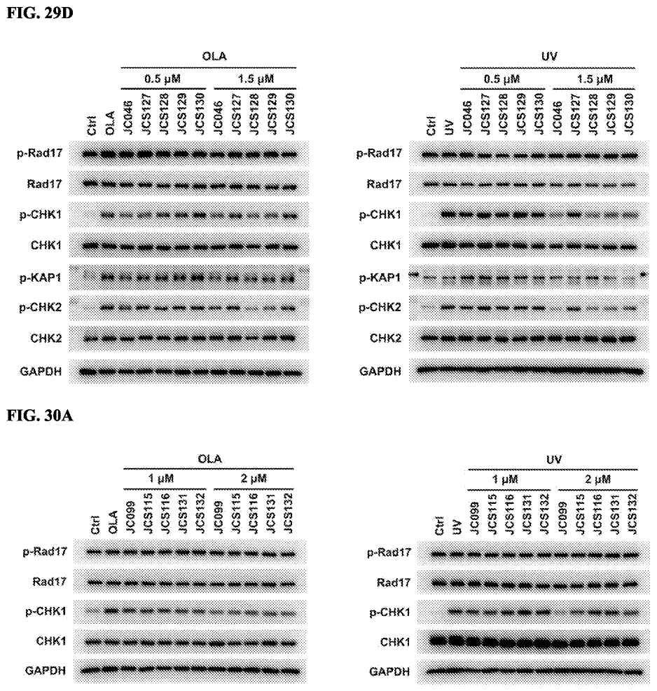

ATR inhibition was assessed in C4-2B/MR cells. Considering ATM is a homologous protein of ATR, we also detected the effect of JC046 analogs on the phosphorylation of KAP1 at Ser 824 and CHK2 at Thr 68 sites, which were used as markers of ATM signaling pathway. As shown in FIG. 29, JCS121 and JCS124 dramatically decreased OLA or UV induced phosphorylation of Rad17 at Ser 645 and CHK1 at Ser 345 sites, which is close to JC046. Although JCS119/126/128/129 also decreased OLA or UV induced phosphorylation of Rad17 and CHK1, their potency was less than JC046. JC046 and its analogs did not affect KAP1 and CHK2 phosphorylation at 0.5 μM, but a decrease of KAP1 and CHK2 phosphorylation were observed at 1.5 μM.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EOUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound having a structure represented by Formula I, Formula II, or Formula III, or a pharmaceutically acceptable salt thereof: 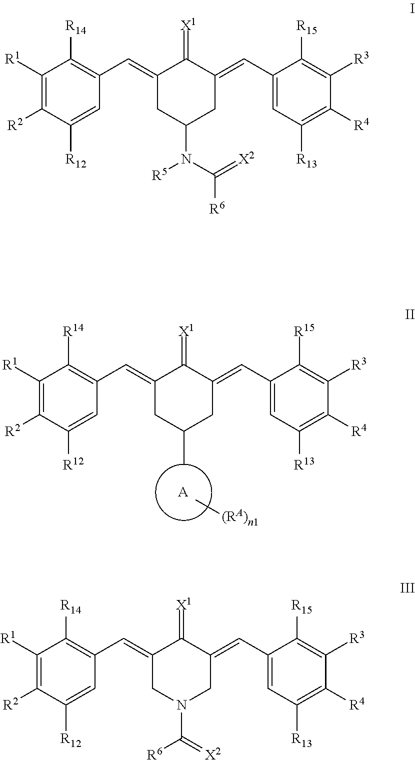
wherein:
A is aryl or heteroaryl;
n1 is 1, 2, 3, 4, or 5;
X¹ is O, S, or NRW;
X² is O, S, or NR⁸;
each of R², R⁴, R¹², R¹³, R¹⁴, and R¹⁵ is independently selected from H, fluoro, chloro, bromo, and iodo;
each of R¹ and R³ is independently selected from H, fluoro, chloro, bromo, iodo, and —O(alkyl);
each of R⁵, R⁷, and R⁸ is independently selected from H, alkyl, and aralkyl;
R^{A} is alkyl, acyl, or amido;
R^{C} is H, alkyl, or acyl; and
R⁶ is alkyl, aryl, heteroaryl, or heterocyclyl.

2. The compound of claim 1, wherein the compound has a structure represented by Formula La, or a pharmaceutically acceptable salt thereof: 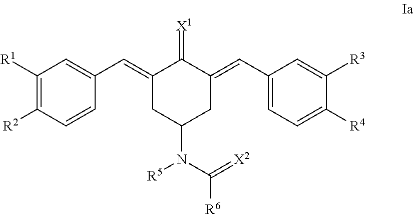
wherein:
X¹ is O, S, or NR;
X² is O, S, or NR⁸;
each of R¹, R², R³, and R⁴ is independently selected from fluoro, chloro, bromo, and iodo;
each of R⁵, R⁷, and R⁸ is independently selected from H, alkyl and aralkyl; and
R⁶ is alkyl, aryl, heteroaryl, or heterocyclyl.

3. The compound of claim 1, wherein the compound has a structure represented by Formula I, or a pharmaceutically acceptable salt thereof:

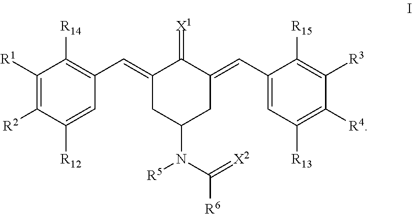

4. The compound of claim 1, wherein the compound has a structure represented by Formula II, or a pharmaceutically acceptable salt thereof:

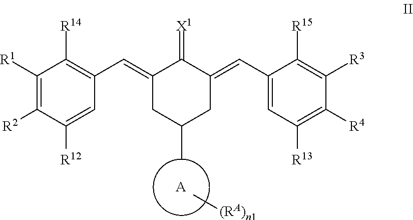

5. The compound of claim 1, wherein the compound has a structure represented by Formula III, or a pharmaceutically acceptable salt thereof:

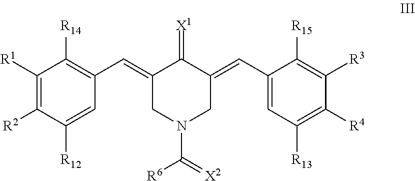

6. The compound of any one of claims 1-5, wherein the compound is not 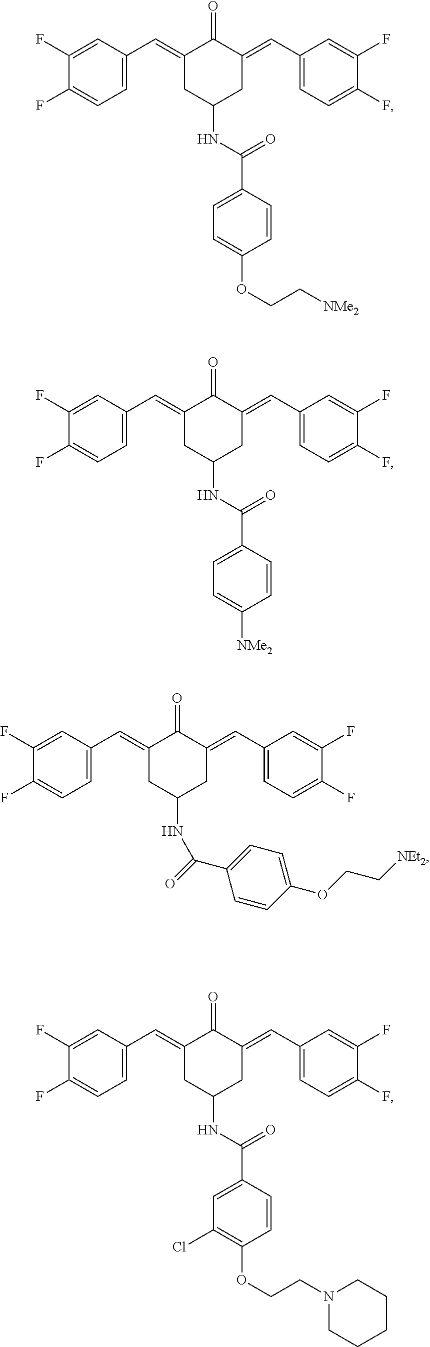 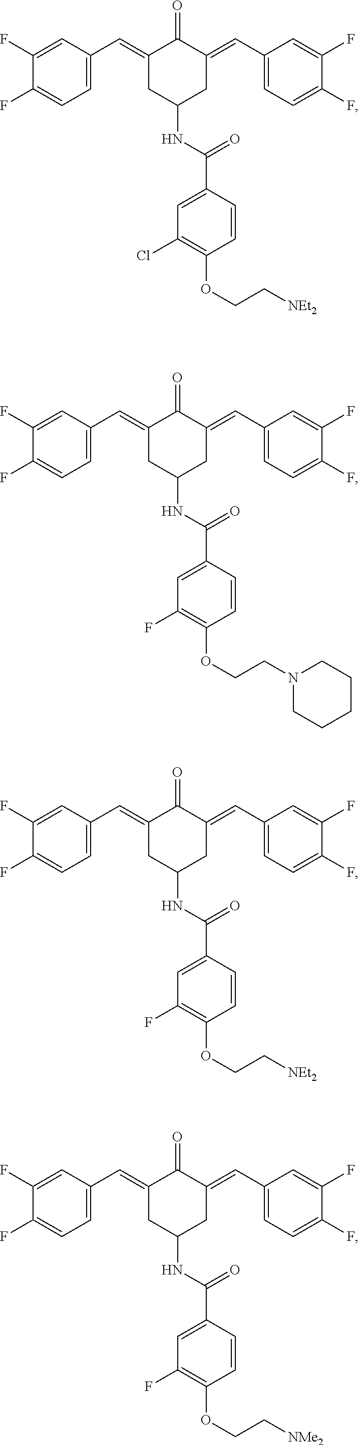 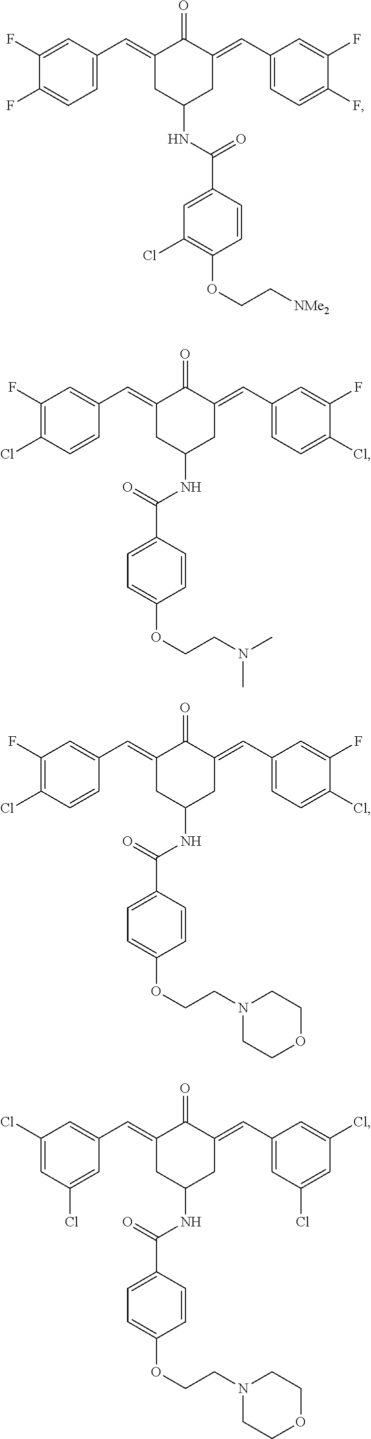 [SMILES: CN(C)CCOc1ccc(C(=O)NC2C/C(=C\c3ccc(F)c(F)c3)C(=O)/C(=C/c3ccc(F)c(F)c3)C2)cc1Cl ; CN(C)CCOc1ccc(C(=O)NC2C/C(=C\c3ccc(Cl)c(F)c3)C(=O)/C(=C/c3ccc(Cl)c(F)c3)C2)cc1 ; O=C1/C(=C/c2ccc(Cl)c(F)c2)CC(NC(=O)c2ccc(OCCN3CCOCC3)cc2)C/C1=C\c1ccc(Cl)c(F)c1 ; O=C1/C(=C/c2cc(Cl)cc(Cl)c2)CC(NC(=O)c2ccc(OCCN3CCOCC3)cc2)C/C1=C\c1cc(Cl)cc(Cl)c1] 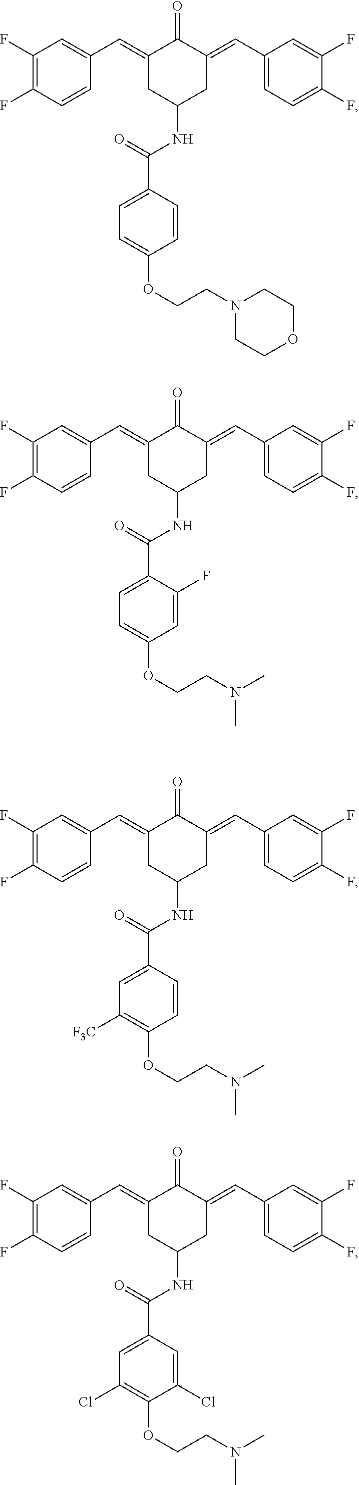 [SMILES: O=C1/C(=C/c2ccc(F)c(F)c2)CC(NC(=O)c2ccc(OCCN3CCOCC3)cc2)C/C1=C\c1ccc(F)c(F)c1 ; CN(C)CCOc1ccc(C(=O)NC2C/C(=C\c3ccc(F)c(F)c3)C(=O)/C(=C/c3ccc(F)c(F)c3)C2)c(F)c1 ; CN(C)CCOc1ccc(C(=O)NC2C/C(=C\c3ccc(F)c(F)c3)C(=O)/C(=C/c3ccc(F)c(F)c3)C2)cc1C(F)(F)F ; CN(C)CCOc1c(Cl)cc(C(=O)NC2C/C(=C\c3ccc(F)c(F)c3)C(=O)/C(=C/c3ccc(F)c(F)c3)C2)cc1Cl] 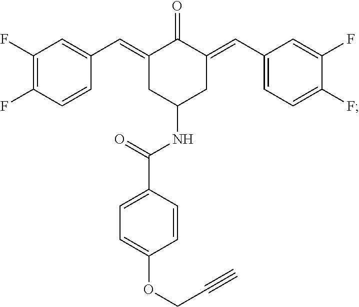 [SMILES: C#CCOc1ccc(C(=O)NC2C/C(=C\c3ccc(F)c(F)c3)C(=O)/C(=C/c3ccc(F)c(F)c3)C2)cc1]
or a pharmaceutically acceptable salt thereof.

7. The compound of any one of claims 1-6, wherein the compound is not 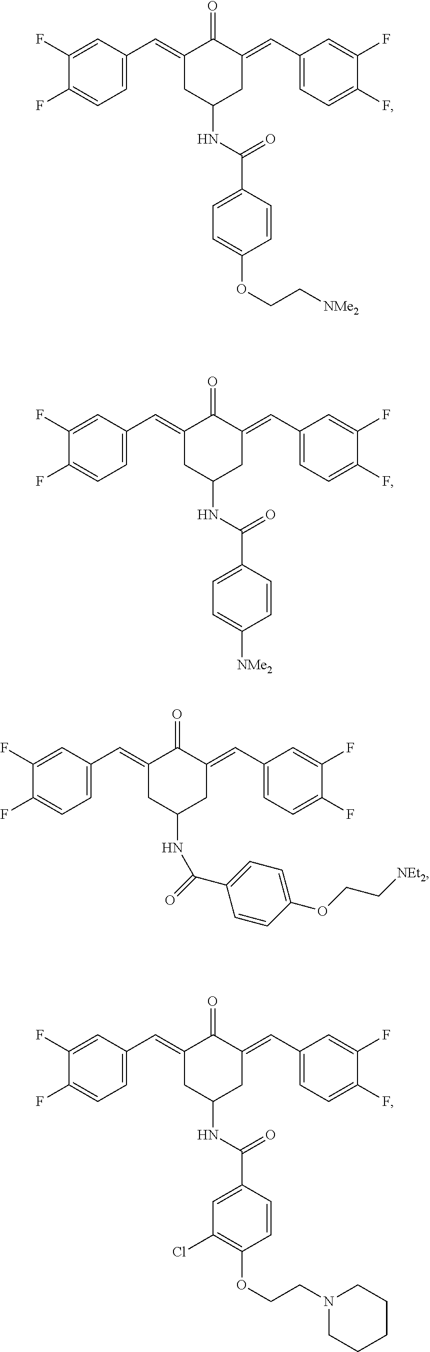 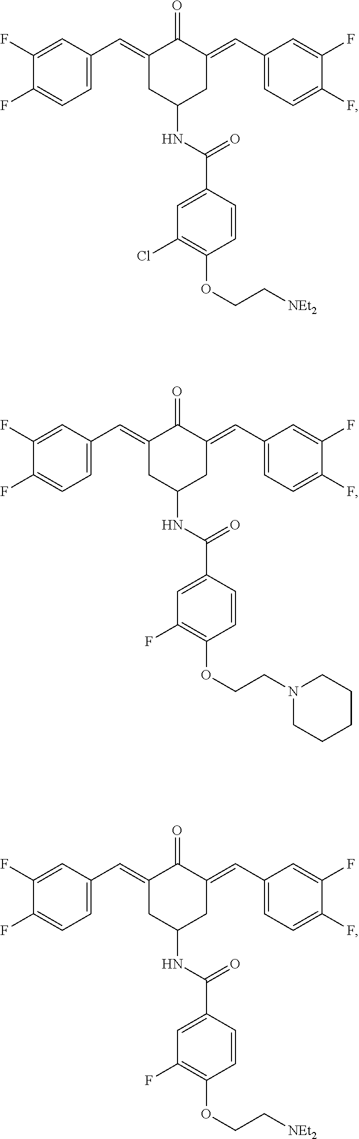 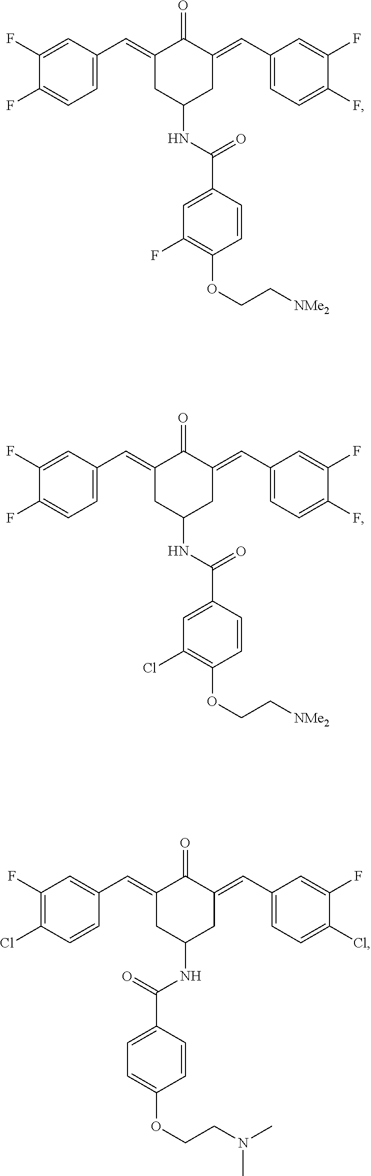 [SMILES: CN(C)CCOc1ccc(C(=O)NC2C/C(=C\c3ccc(F)c(F)c3)C(=O)/C(=C/c3ccc(F)c(F)c3)C2)cc1F ; CN(C)CCOc1ccc(C(=O)NC2C/C(=C\c3ccc(F)c(F)c3)C(=O)/C(=C/c3ccc(F)c(F)c3)C2)cc1Cl ; CN(C)CCOc1ccc(C(=O)NC2C/C(=C\c3ccc(Cl)c(F)c3)C(=O)/C(=C/c3ccc(Cl)c(F)c3)C2)cc1] 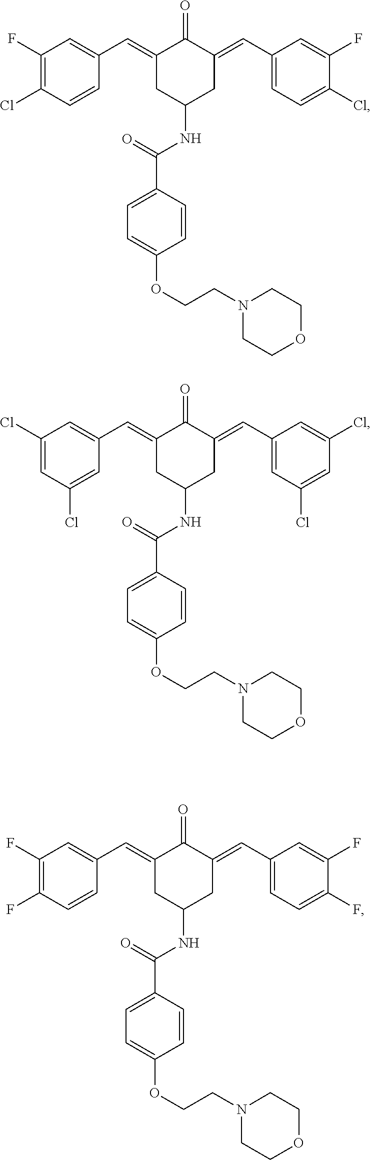 [SMILES: O=C1/C(=C/c2ccc(Cl)c(F)c2)CC(NC(=O)c2ccc(OCCN3CCOCC3)cc2)C/C1=C\c1ccc(Cl)c(F)c1 ; O=C1/C(=C/c2cc(Cl)cc(Cl)c2)CC(NC(=O)c2ccc(OCCN3CCOCC3)cc2)C/C1=C\c1cc(Cl)cc(Cl)c1 ; O=C1/C(=C/c2ccc(F)c(F)c2)CC(NC(=O)c2ccc(OCCN3CCOCC3)cc2)C/C1=C\c1ccc(F)c(F)c1] 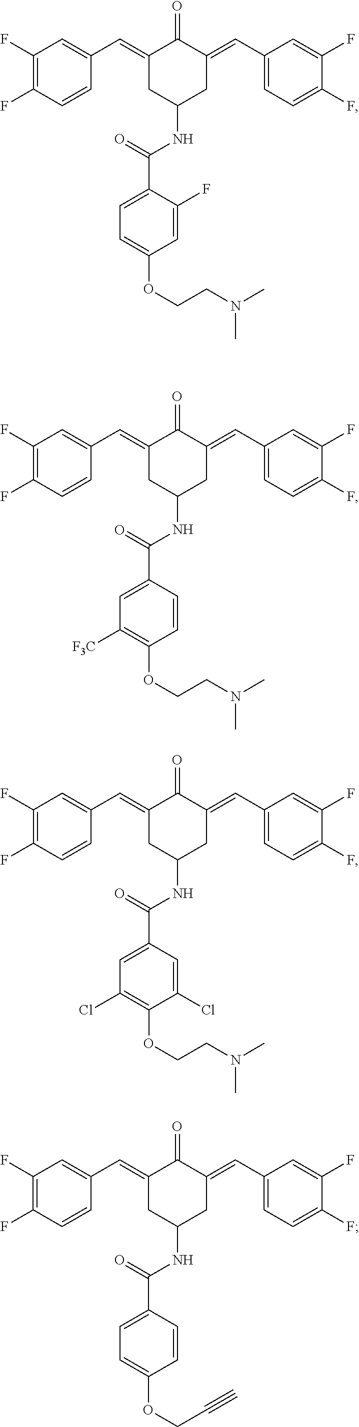 [SMILES: CN(C)CCOc1ccc(C(=O)NC2C/C(=C\c3ccc(F)c(F)c3)C(=O)/C(=C/c3ccc(F)c(F)c3)C2)c(F)c1 ; CN(C)CCOc1ccc(C(=O)NC2C/C(=C\c3ccc(F)c(F)c3)C(=O)/C(=C/c3ccc(F)c(F)c3)C2)cc1C(F)(F)F ; CN(C)CCOc1c(Cl)cc(C(=O)NC2C/C(=C\c3ccc(F)c(F)c3)C(=O)/C(=C/c3ccc(F)c(F)c3)C2)cc1Cl ; C#CCOc1ccc(C(=O)NC2C/C(=C\c3ccc(F)c(F)c3)C(=O)/C(=C/c3ccc(F)c(F)c3)C2)cc1] 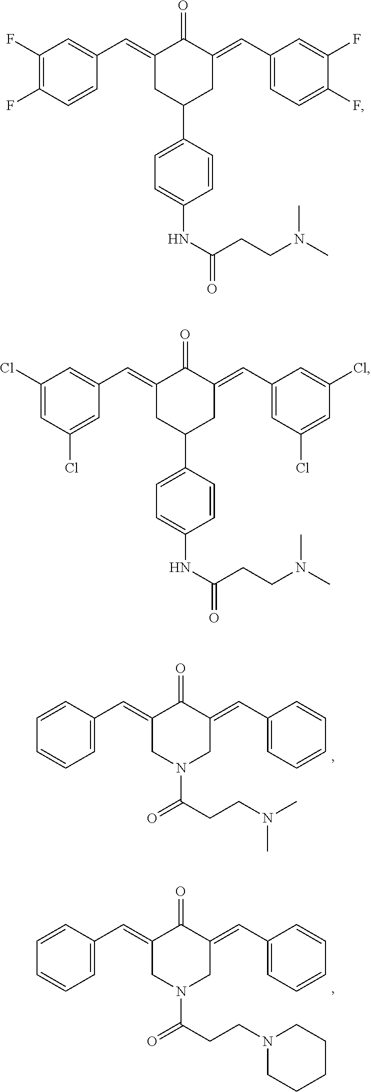 [SMILES: CN(C)CCC(=O)Nc1ccc(C2C/C(=C\c3ccc(F)c(F)c3)C(=O)/C(=C/c3ccc(F)c(F)c3)C2)cc1 ; CN(C)CCC(=O)Nc1ccc(C2C/C(=C\c3cc(Cl)cc(Cl)c3)C(=O)/C(=C/c3cc(Cl)cc(Cl)c3)C2)cc1 ; CN(C)CCC(=O)N1C/C(=C\c2ccccc2)C(=O)/C(=C/c2ccccc2)C1 ; O=C1/C(=C/c2ccccc2)CN(C(=O)CCN2CCCCC2)C/C1=C\c1ccccc1] 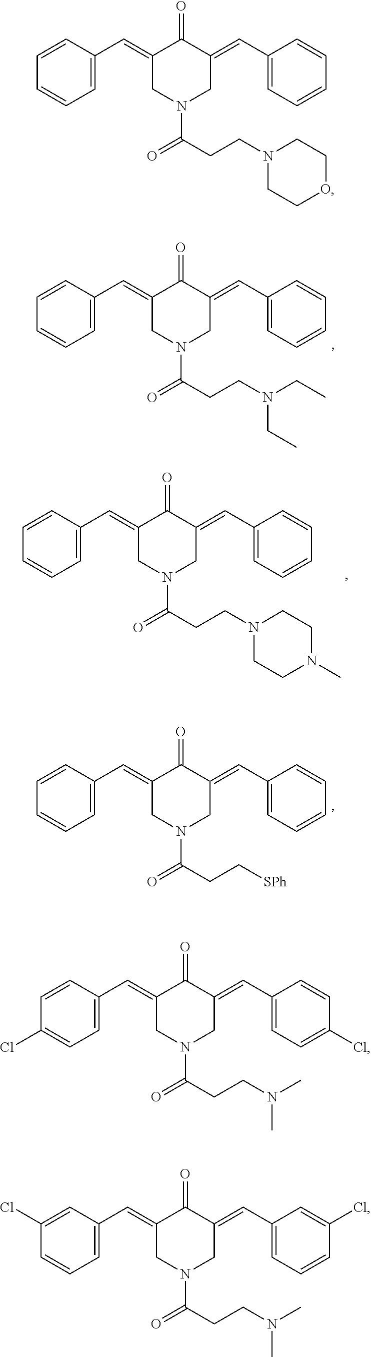 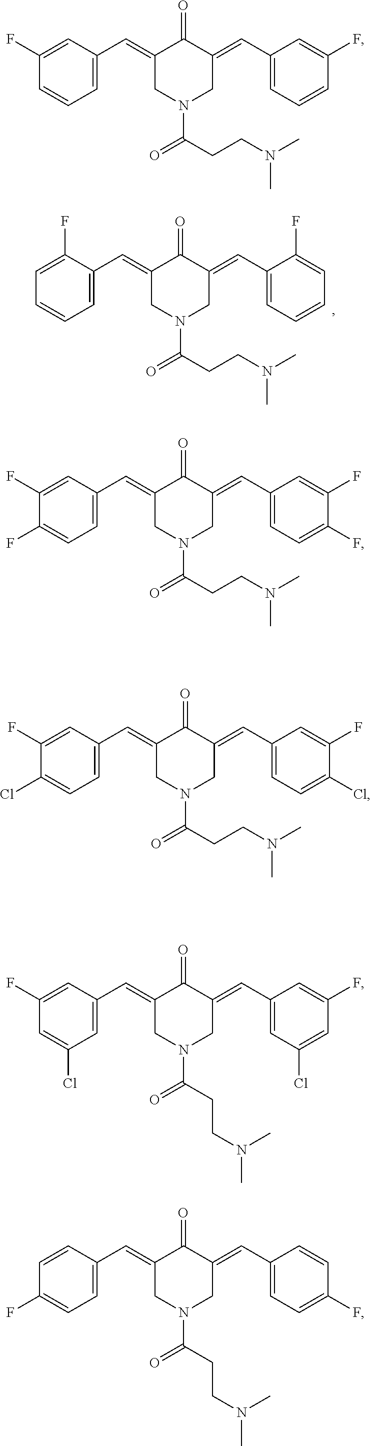 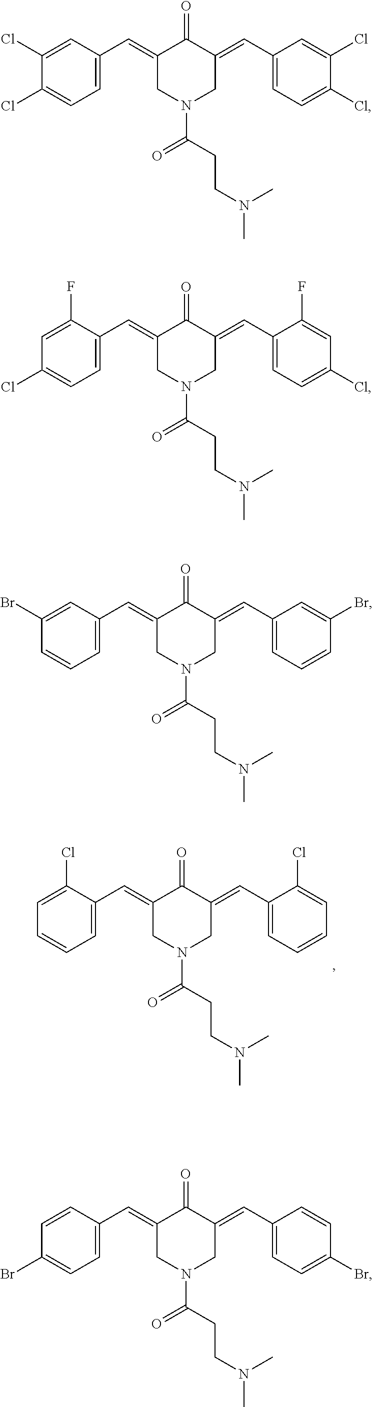 [SMILES: CN(C)CCC(=O)N1C/C(=C\c2ccc(Cl)c(Cl)c2)C(=O)/C(=C/c2ccc(Cl)c(Cl)c2)C1 ; CN(C)CCC(=O)N1C/C(=C\c2ccc(Cl)cc2F)C(=O)/C(=C/c2ccc(Cl)cc2F)C1 ; CN(C)CCC(=O)N1C/C(=C\c2cccc(Br)c2)C(=O)/C(=C/c2cccc(Br)c2)C1 ; CN(C)CCC(=O)N1C/C(=C\c2ccccc2Cl)C(=O)/C(=C/c2ccccc2Cl)C1 ; CN(C)CCC(=O)N1C/C(=C\c2ccc(Br)cc2)C(=O)/C(=C/c2ccc(Br)cc2)C1] 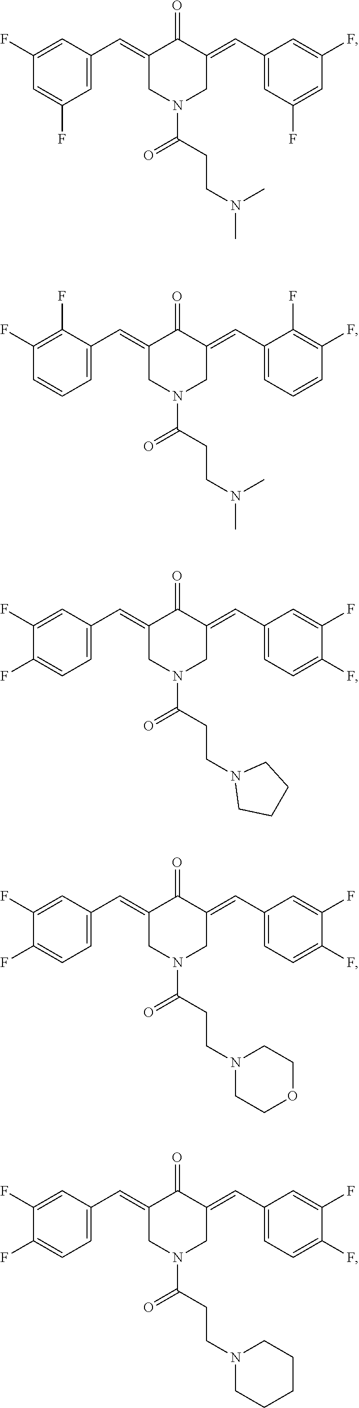 [SMILES: CN(C)CCC(=O)N1C/C(=C\c2cc(F)cc(F)c2)C(=O)/C(=C/c2cc(F)cc(F)c2)C1 ; CN(C)CCC(=O)N1C/C(=C\c2cccc(F)c2F)C(=O)/C(=C/c2cccc(F)c2F)C1 ; O=C1/C(=C/c2ccc(F)c(F)c2)CN(C(=O)CCN2CCCC2)C/C1=C\c1ccc(F)c(F)c1 ; O=C1/C(=C/c2ccc(F)c(F)c2)CN(C(=O)CCN2CCOCC2)C/C1=C\c1ccc(F)c(F)c1 ; O=C1/C(=C/c2ccc(F)c(F)c2)CN(C(=O)CCN2CCCCC2)C/C1=C\c1ccc(F)c(F)c1] 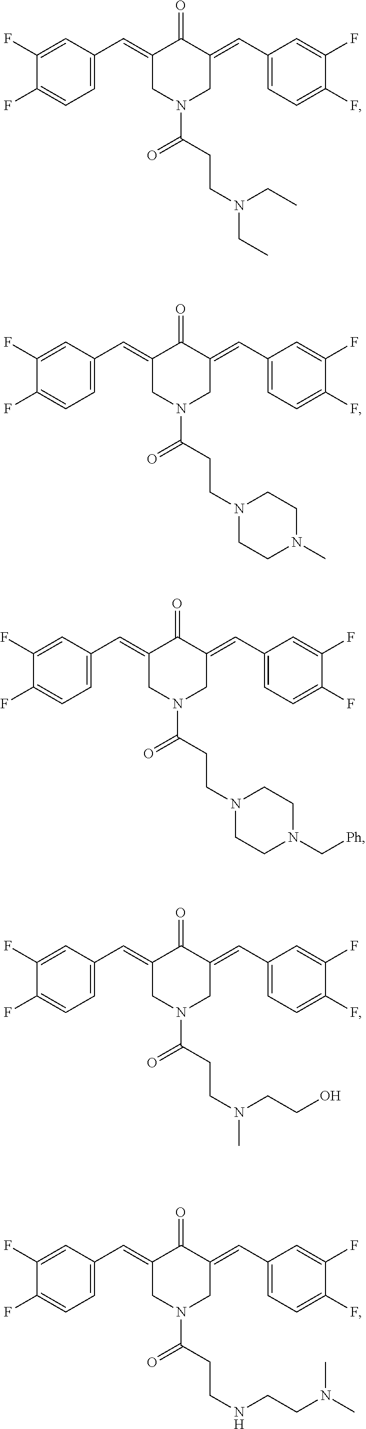 [SMILES: CCN(CC)CCC(=O)N1C/C(=C\c2ccc(F)c(F)c2)C(=O)/C(=C/c2ccc(F)c(F)c2)C1 ; CN1CCN(CCC(=O)N2C/C(=C\c3ccc(F)c(F)c3)C(=O)/C(=C/c3ccc(F)c(F)c3)C2)CC1 ; O=C1/C(=C/c2ccc(F)c(F)c2)CN(C(=O)CCN2CCN(Cc3ccccc3)CC2)C/C1=C\c1ccc(F)c(F)c1 ; CN(CCO)CCC(=O)N1C/C(=C\c2ccc(F)c(F)c2)C(=O)/C(=C/c2ccc(F)c(F)c2)C1 ; CN(C)CCNCCC(=O)N1C/C(=C\c2ccc(F)c(F)c2)C(=O)/C(=C/c2ccc(F)c(F)c2)C1] 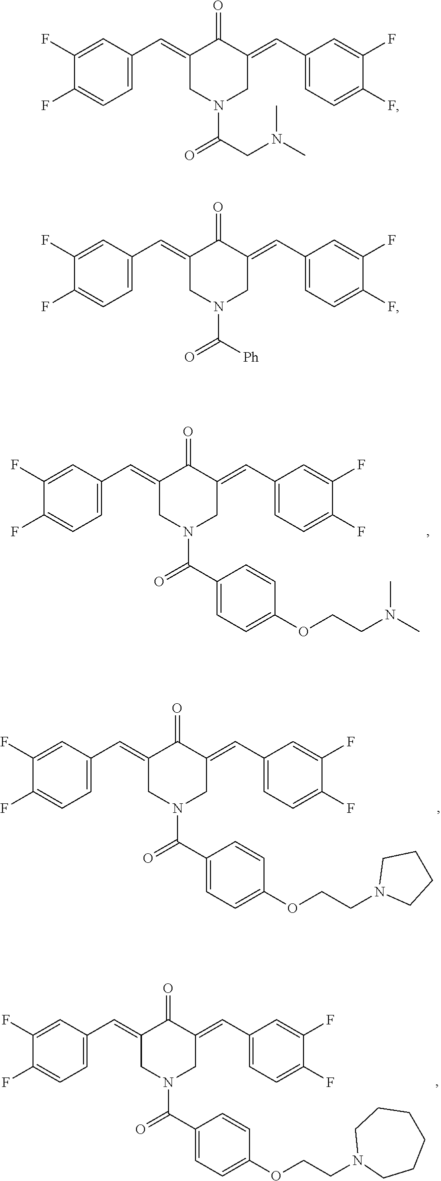 [SMILES: CN(C)CC(=O)N1C/C(=C\c2ccc(F)c(F)c2)C(=O)/C(=C/c2ccc(F)c(F)c2)C1 ; O=C1/C(=C/c2ccc(F)c(F)c2)CN(C(=O)c2ccccc2)C/C1=C\c1ccc(F)c(F)c1 ; CN(C)CCOc1ccc(C(=O)N2C/C(=C\c3ccc(F)c(F)c3)C(=O)/C(=C/c3ccc(F)c(F)c3)C2)cc1 ; O=C1/C(=C/c2ccc(F)c(F)c2)CN(C(=O)c2ccc(OCCN3CCCC3)cc2)C/C1=C\c1ccc(F)c(F)c1 ; O=C1/C(=C/c2ccc(F)c(F)c2)CN(C(=O)c2ccc(OCCN3CCCCCC3)cc2)C/C1=C\c1ccc(F)c(F)c1] 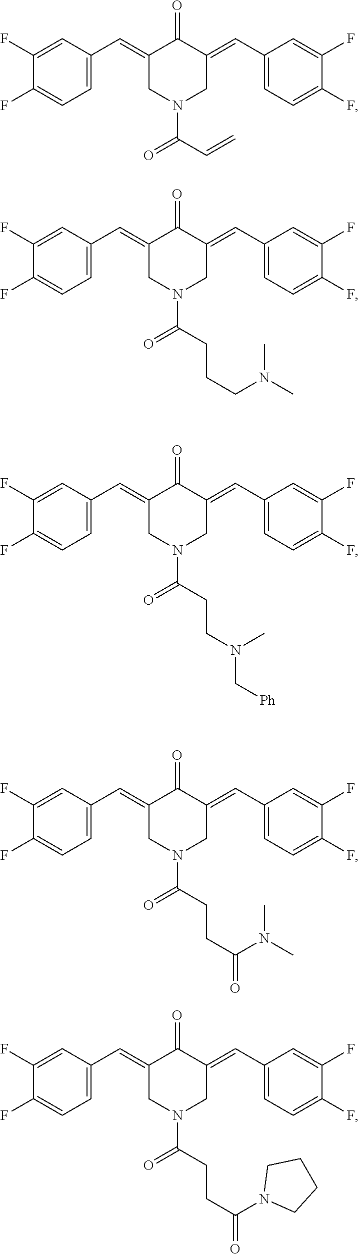 [SMILES: C=CC(=O)N1C/C(=C\c2ccc(F)c(F)c2)C(=O)/C(=C/c2ccc(F)c(F)c2)C1 ; CN(C)CCCC(=O)N1C/C(=C\c2ccc(F)c(F)c2)C(=O)/C(=C/c2ccc(F)c(F)c2)C1 ; CN(CCC(=O)N1C/C(=C\c2ccc(F)c(F)c2)C(=O)/C(=C/c2ccc(F)c(F)c2)C1)Cc1ccccc1 ; CN(C)C(=O)CCC(=O)N1C/C(=C\c2ccc(F)c(F)c2)C(=O)/C(=C/c2ccc(F)c(F)c2)C1 ; O=C1/C(=C/c2ccc(F)c(F)c2)CN(C(=O)CCC(=O)N2CCCC2)C/C1=C\c1ccc(F)c(F)c1] 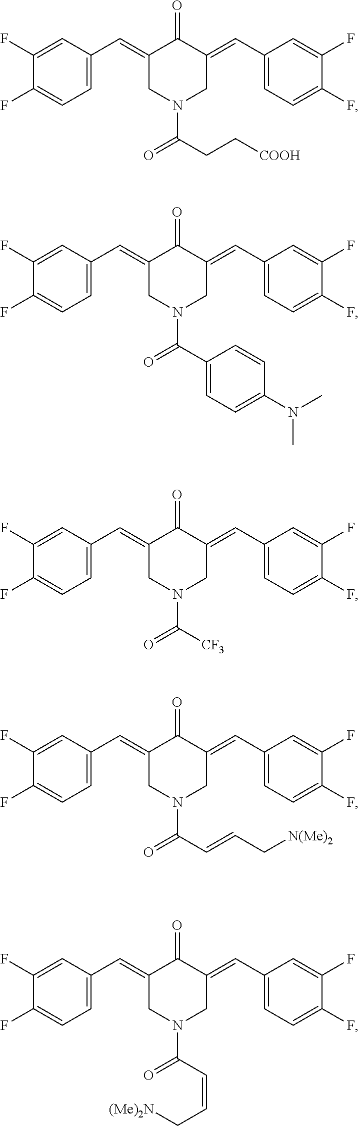 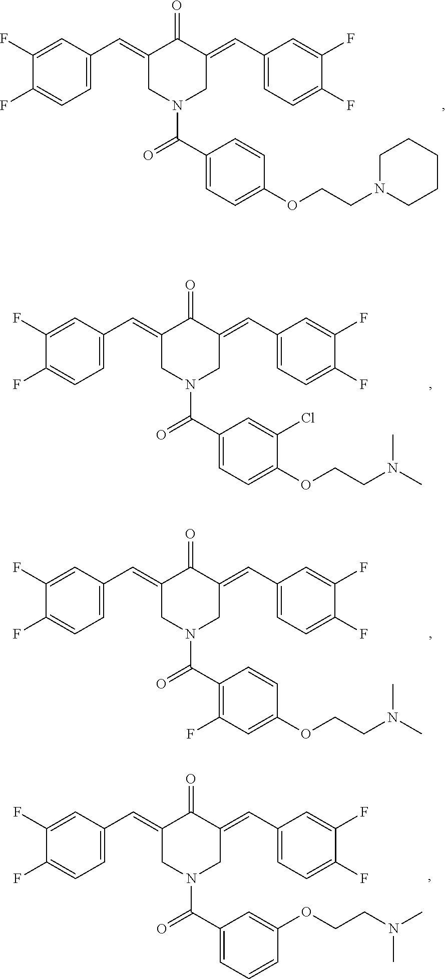 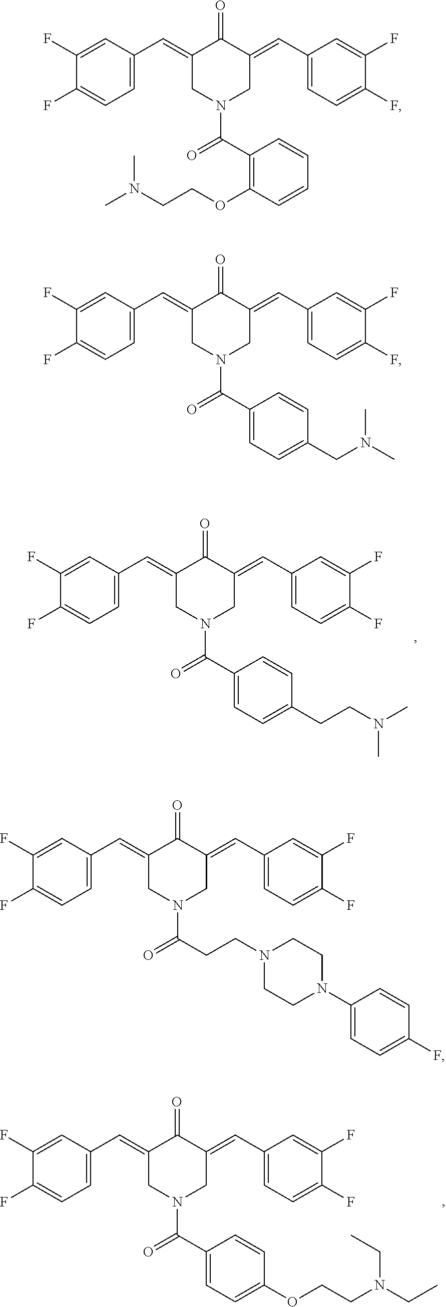 [SMILES: CN(C)CCOc1ccccc1C(=O)N1C/C(=C\c2ccc(F)c(F)c2)C(=O)/C(=C/c2ccc(F)c(F)c2)C1 ; CN(C)Cc1ccc(C(=O)N2C/C(=C\c3ccc(F)c(F)c3)C(=O)/C(=C/c3ccc(F)c(F)c3)C2)cc1 ; CN(C)CCc1ccc(C(=O)N2C/C(=C\c3ccc(F)c(F)c3)C(=O)/C(=C/c3ccc(F)c(F)c3)C2)cc1 ; O=C1/C(=C/c2ccc(F)c(F)c2)CN(C(=O)CCN2CCN(c3ccc(F)cc3)CC2)C/C1=C\c1ccc(F)c(F)c1 ; CCN(CC)CCOc1ccc(C(=O)N2C/C(=C\c3ccc(F)c(F)c3)C(=O)/C(=C/c3ccc(F)c(F)c3)C2)cc1] 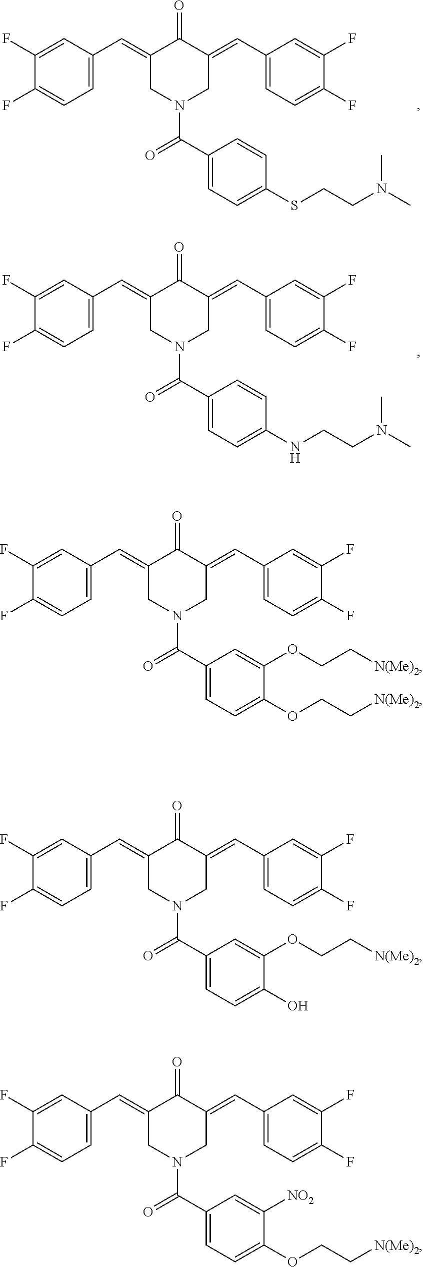 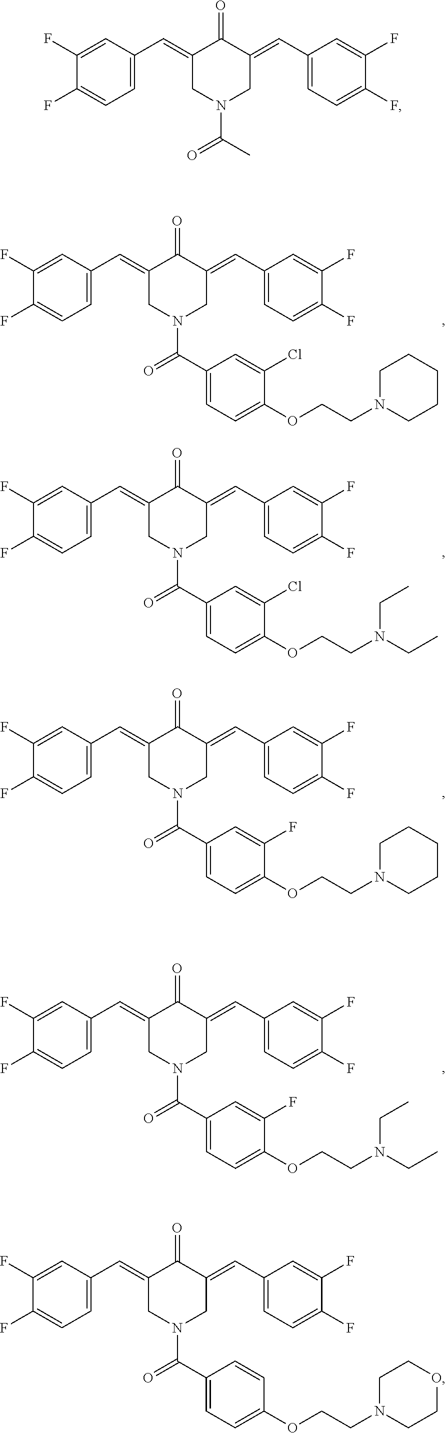 [SMILES: CC(=O)N1C/C(=C\c2ccc(F)c(F)c2)C(=O)/C(=C/c2ccc(F)c(F)c2)C1 ; O=C1/C(=C/c2ccc(F)c(F)c2)CN(C(=O)c2ccc(OCCN3CCCCC3)c(Cl)c2)C/C1=C\c1ccc(F)c(F)c1 ; CCN(CC)CCOc1ccc(C(=O)N2C/C(=C\c3ccc(F)c(F)c3)C(=O)/C(=C/c3ccc(F)c(F)c3)C2)cc1Cl ; O=C1/C(=C/c2ccc(F)c(F)c2)CN(C(=O)c2ccc(OCCN3CCCCC3)c(F)c2)C/C1=C\c1ccc(F)c(F)c1 ; CCN(CC)CCOc1ccc(C(=O)N2C/C(=C\c3ccc(F)c(F)c3)C(=O)/C(=C/c3ccc(F)c(F)c3)C2)cc1F ; O=C1/C(=C/c2ccc(F)c(F)c2)CN(C(=O)c2ccc(OCCN3CCOCC3)cc2)C/C1=C\c1ccc(F)c(F)c1] 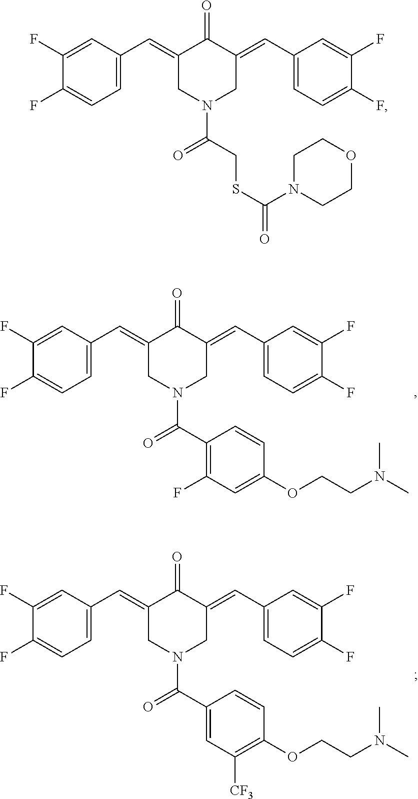 [SMILES: O=C1/C(=C/c2ccc(F)c(F)c2)CN(C(=O)CSC(=O)N2CCOCC2)C/C1=C\c1ccc(F)c(F)c1 ; CN(C)CCOc1ccc(C(=O)N2C/C(=C\c3ccc(F)c(F)c3)C(=O)/C(=C/c3ccc(F)c(F)c3)C2)c(F)c1 ; CN(C)CCOc1ccc(C(=O)N2C/C(=C\c3ccc(F)c(F)c3)C(=O)/C(=C/c3ccc(F)c(F)c3)C2)cc1C(F)(F)F]
or a pharmaceutically acceptable salt thereof.

8. The compound of any one of claims 1-7, wherein R¹ is fluoro.

9. The compound of any one of claims 1-7, wherein R¹ is chloro.

10. The compound of any one of claims 1-7, wherein R¹ is H.

11. The compound of any one of claims 1-7, wherein R¹ is —O(alkyl) (e.g., methoxy).

12. The compound of any one of claims 1-11, wherein R² is fluoro.

13. The compound of any one of claims 1-11, wherein R² is chloro.

14. The compound of any one of claims 1-11, wherein R² is H.

15. The compound of any one of claims 1-14, wherein R³ is fluoro.

16. The compound of any one of claims 1-14, wherein R³ is chloro.

17. The compound of any one of claims 1-14, wherein R³ is H.

18. The compound of any one of claims 1-14, wherein R³ is —O(alkyl) (e.g., methoxy).

19. The compound of any one of claims 1-18, wherein R⁴ is fluoro.

20. The compound of any one of claims 1-18, wherein R⁴ is chloro.

21. The compound of any one of claims 1-18, wherein R⁴ is H.

22. The compound of any one of claims 1 and 3-21, wherein R¹² is chloro.

23. The compound of any one of claims 1 and 3-21, wherein R¹² is fluoro.

24. The compound of any one of claims 1 and 3-21, wherein R¹² is H.

25. The compound of any one of claims 1 and 3-24, wherein R¹³ is chloro.

26. The compound of any one of claims 1 and 3-24, wherein R¹³ is fluoro.

27. The compound of any one of claims 1 and 3-24, wherein R¹³ is H.

28. The compound of any one of claims 1 and 3-27, wherein R¹⁴ is chloro.

29. The compound of any one of claims 1 and 3-27, wherein R¹⁴ is fluoro.

30. The compound of any one of claims 1 and 3-27, wherein R¹⁴ is H.

31. The compound of any one of claims 1 and 3-30, wherein R¹⁵ is chloro.

32. The compound of any one of claims 1 and 3-30, wherein R¹⁵ is fluoro.

33. The compound of any one of claims 1 and 3-30, wherein R¹⁵ is H.

34. The compound of any one of claims 1-33, wherein X¹ is O.

35. The compound of any one of claims 1, 4, or 6-34, wherein A is heteroaryl (e.g., indazolyl.)

36. The compound of any one of claims 1, 4, or 6-35, wherein n1 is 1.

37. The compound of any one of claims 1, 4, or 6-36 wherein R^{A} is alkylamido (e.g., N-diethylamido).

38. The compound of any one of claims 1-3 or 5-37, wherein X² is O.

39. The compound of any one of claims 1-3 or 5-38, wherein R⁵ is H.

40. The compound of any one of claims 1-3 or 6-39 wherein R⁶ is alkyl.

41. The compound of any one of claims 1-3 or 6-39, wherein R⁶ is heteroaryl (e.g., pyridinyl, pyrimidinyl, indolyl, or pyrazolopyridinyl).

42. The compound of any one of claims 1-3 or 6-39, wherein R⁶ is heteroaryl (e.g., pyridinyl, pyrimidinyl, indolyl, quinazolinyl, phthalazinyl, or pyrazolopyridinyl).

43. The compound of claim 42, wherein R⁶ is pyridinyl or pyrimidinyl.

44. The compound of any one of claims 1-3 or 6-39, wherein R⁶ is aryl.

45. The compound of any one of claims 1-3 or 6-44, wherein R⁶ is substituted with alkyl, alkenyl, alkynyl, halo, hydroxyl, carboxyl, acyl, acetyl, ester, thioester, alkoxy, phosphoryl, amino, amido, cyano, nitro, azido, alkylthio, cycloalkyl, alkylsulfonyl, sulfonamide, cycloalkyl, aryl, heteroaryl, and heterocyclyl.

46. The compound of claim 45, wherein R⁶ is substituted with heterocyclyl (e.g., pyrrolidinyl pyrrolidinyl, piperazinyl, piperidinyl, or (1-ethylpyrrolidin-2-yl)methanamine).

47. The compound of claim 45, wherein R⁶ is substituted with pyrrolidinyl or (1-ethylpyrrolidin-2-yl)methanamine).

48. The compound of any one of claims 45-47, wherein R⁶ is substituted with alkylamino (e.g., —N(CH₂)₂N(C₄H₈)).

49. The compound of any one of claims 45-48, wherein R⁶ is substituted with alkylamido (e.g., N-diethylamido).

50. The compound of any one of claims 1-3 or 6-49, wherein R⁶ is substituted with nitro.

51. The compound of any one of claims 1-3 or 6-50, wherein R⁶ is substituted with alkoxy.

52. The compound of any one of claims 1-3 or 6-51, wherein R⁶ is substituted with amino (e.g., —NH₂).

53. The compound of any one of claims 50-52, wherein R⁶ is substituted with heterocyclyl.

54. The compound of any one of claims 1-3 or 6-7, wherein the compound has a structure represented by formula Ib or a pharmaceutically acceptable salt thereof: 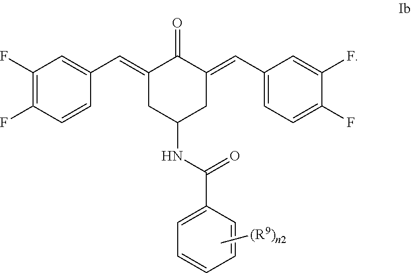
wherein
each R⁹ is independently selected from alkyl, alkenyl, alkynyl, halo, hydroxyl, carboxyl, acyl, acetyl, ester, thioester, alkoxy, phosphoryl, amino, amido, cyano, nitro, azido, alkylthio, cycloalkyl, alkylsulfonyl, sulfonamide, cycloalkyl, aryl, heteroaryl, and heterocyclyl; and
n is 1, 2, 3, 4, or 5.

55. The compound of any one of claims 1-39, wherein the compound has a structure represented by Formula Ic, Formula IIa, or Formula IIIa, or a pharmaceutically acceptable salt thereof: 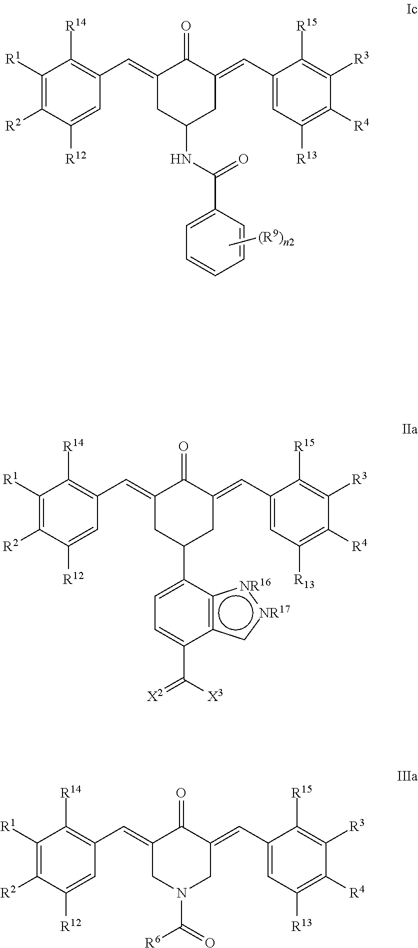
wherein
X³ is alkyl or N(R¹⁸)₂;
each R⁹ is independently selected from alkyl, alkenyl, alkynyl, halo, hydroxyl, carboxyl, acyl, acetyl, ester, thioester, alkoxy, phosphoryl, amino, amido, cyano, nitro, azido, alkylthio, cycloalkyl, alkylsulfonyl, sulfonamide, cycloalkyl, aryl, heteroaryl, and heterocyclyl;
each R¹⁶, R¹⁷, and R¹⁸ is independently H or alkyl; and
n2 is 1, 2, 3, 4, or 5.

56. The compound of claim 55, wherein the compound has a structure represented by Formula Ic, or a pharmaceutically acceptable salt thereof:

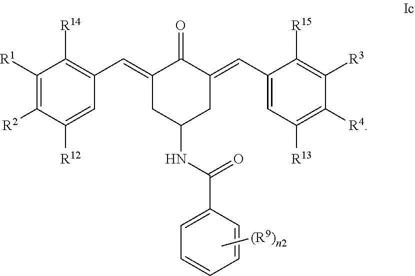

57. The compound of claim 55, wherein the compound has a structure represented by Formula IIa, or a pharmaceutically acceptable salt thereof:

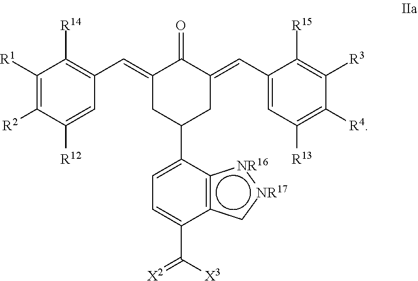

58. The compound of claim 55, wherein the compound has a structure represented by Formula IIIa, or a pharmaceutically acceptable salt thereof:

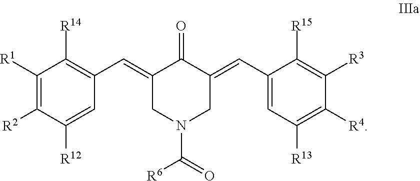

59. The compound of claim 55, wherein the compound has a structure represented by Formula Id, Formula IIb, or Formula IIIb, or a pharmaceutically acceptable salt thereof:

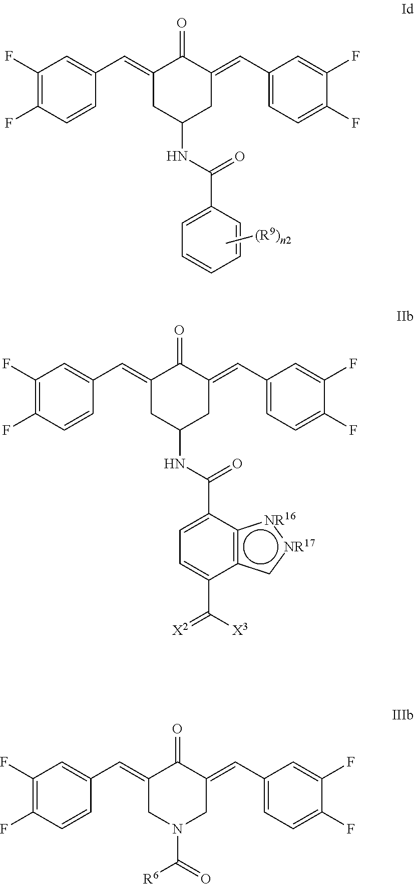

60. The compound of claim 59, wherein the compound has a structure represented by Formula Id, or a pharmaceutically acceptable salt thereof:

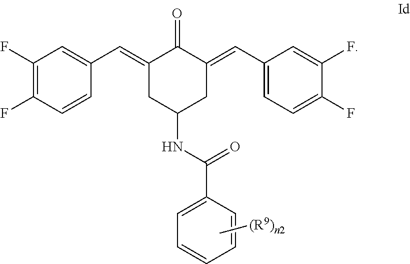

61. The compound of claim 59, wherein the compound has a structure represented by Formula IIb, or a pharmaceutically acceptable salt thereof:

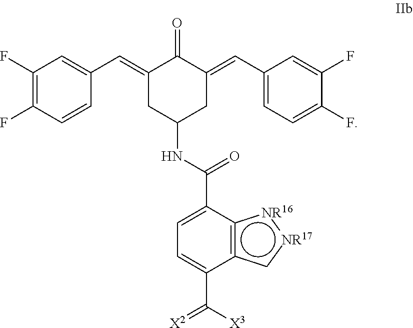

62. The compound of claim 59, wherein the compound has a structure represented by Formula IIIb, or a pharmaceutically acceptable salt thereof:

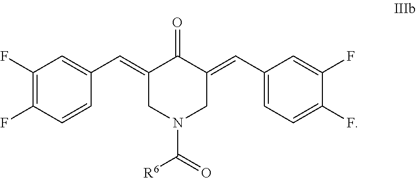

63. The compound of claim 59, wherein the compound has a structure represented by formula Ie or a pharmaceutically acceptable salt thereof: 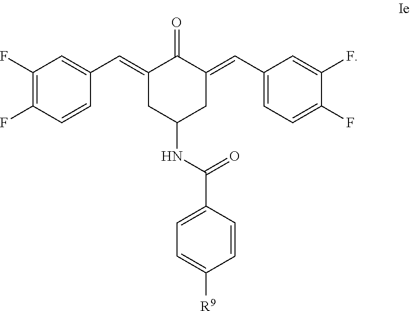
wherein
R⁹ is selected from alkyl, alkenyl, alkynyl, halo, hydroxyl, carboxyl, acyl, acetyl, ester, thioester, alkoxy, phosphoryl, amino, alkylamino, amido, cyano, nitro, azido, alkylthio, cycloalkyl, alkylsulfonyl, sulfonamide, carbamoylamino, cycloalkyl, aryl, heteroaryl, and heterocyclyl.

64. The compound of claim 59, wherein the compound has a structure represented by Formula If, Formula IIc, or Formula IIIc, or a pharmaceutically acceptable salt thereof: 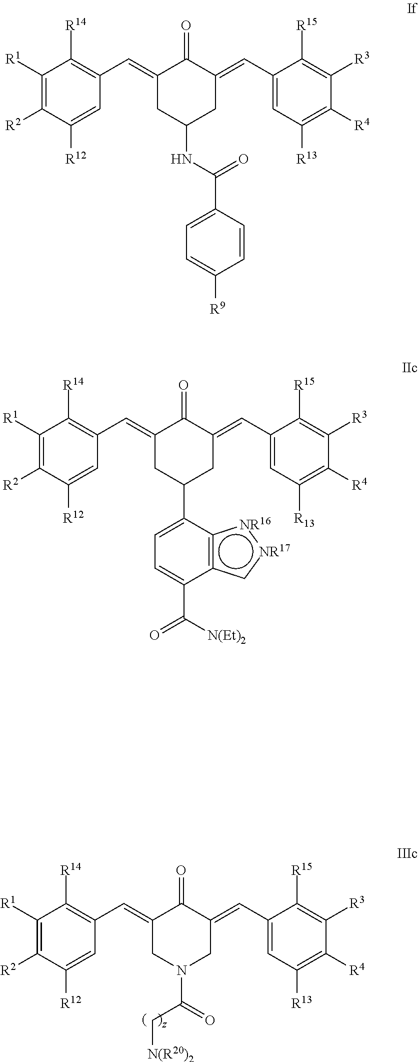
wherein
z is 0, 1, or 2;
R⁹ is selected from alkyl, alkenyl, alkynyl, halo, hydroxyl, carboxyl, acyl, acetyl, ester, thioester, alkoxy, phosphoryl, amino, alkylamino, amido, cyano, nitro, azido, alkylthio, cycloalkyl, alkylsulfonyl, sulfonamide, carbamoylamino, cycloalkyl, aryl, heteroaryl, and heterocyclyl; and
R²⁰ is selected from H, cycloalkyl, cycloalkenyl, heterocycloalkyl, and heterocyclyl.

65. The compound of claim 64, wherein the compound has a structure represented by Formula If, or a pharmaceutically acceptable salt thereof:

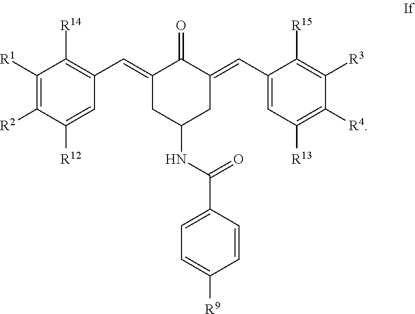

66. The compound of claim 64, wherein the compound has a structure represented by Formula IIc, or a pharmaceutically acceptable salt thereof:

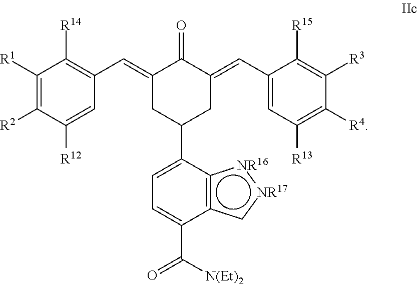

67. The compound of claim 64, wherein the compound has a structure represented by Formula IIIc, or a pharmaceutically acceptable salt thereof:

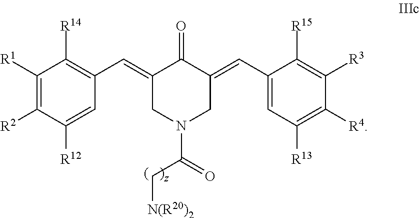

68. The compound of any one of claims 64-67, wherein R¹ is fluoro.

69. The compound of any one of claims 64-68, wherein R² is fluoro.

70. The compound of any one of claims 64-69, wherein R³ is fluoro.

71. The compound of any one of claims 64-70, wherein R⁴ is fluoro.

72. The compound of any one of claims 64-71, wherein R¹² is fluoro.

73. The compound of any one of claims 64-72, wherein R¹³ is fluoro.

74. The compound of any one of claims 64-73, wherein R¹⁴ is fluoro.

75. The compound of any one of claims 64-74, wherein R¹⁵ is fluoro.

76. The compound of claim 64 or 65, wherein R⁹ is alkoxy.

77. The compound of claim 64 or 65, wherein R⁹ is amino.

78. The compound of claim 64 or 65, wherein R⁹ is aryl (e.g., phenyl) or heterocyclyl (e.g., pyrrolidinyl or as N-methyl piperazinyl).

79. The compound of claim 64 or 65, wherein R⁹ is alkylamino.

80. The compound of claim 64 or 66, wherein R¹⁶ is H.

81. The compound of claim 64 or 66, wherein R¹⁶ is alkyl (e.g., methyl).

82. The compound of claim 64 or 66, wherein R¹⁷ is H.

83. The compound of claim 64 or 66, wherein R¹⁷ is alkyl (e.g., methyl).

84. The compound of claim 64 or 67, wherein z is 1.

85. The compound of claim 64 or 67, wherein z is 2.

86. The compound of claim 64 or 65, wherein the compound has a structure represented by formula Ig or a pharmaceutically acceptable salt thereof: 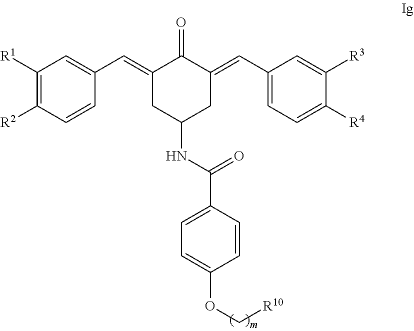
wherein R¹⁰ is selected from alkyl, alkenyl, alkynyl, halo, hydroxyl, carboxyl, acyl, acetyl, ester, thioester, alkoxy, phosphoryl, amino, alkylamino, amido, carbamyl, cyano, nitro, azido, alkylthio, cycloalkyl, alkylsulfonyl, sulfonamide, carbamoylamino, cycloalkyl, aryl, heteroaryl, and heterocyclyl; and
m is 1, 2, 3, 4, 5, 6, 7, 8, 9, or 10.

87. The compound of claim 64, wherein the compound has a structure represented by Formula Ih or Formula IIId, or a pharmaceutically acceptable salt thereof: 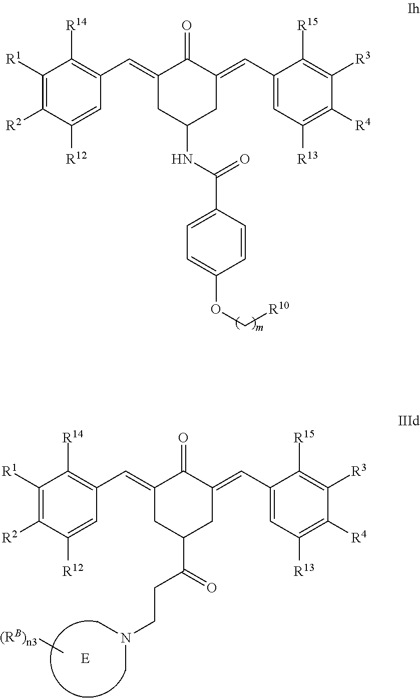
wherein
E is heterocyclyl;
R^{B} is selected from alkyl, amino, hydroxyl, halo, alkoxy, and sulfonyl;
R¹⁰ is selected from alkyl, alkenyl, alkynyl, halo, hydroxyl, carboxyl, acyl, acetyl, ester, thioester, alkoxy, phosphoryl, amino, alkylamino, amido, carbamyl, cyano, nitro, azido, alkylthio, cycloalkyl, alkylsulfonyl, sulfonamide, carbamoylamino, cycloalkyl, aryl, heteroaryl, and heterocyclyl;
n3 is 0, 1, 2, 3, 4, 5, 6, 7, 8, 9, or 10; and
m is 1, 2, 3, 4, 5, 6, 7, 8, 9, or 10.

88. The compound of claim 87, wherein the compound has a structure represented by Formula Ih, or a pharmaceutically acceptable salt thereof:

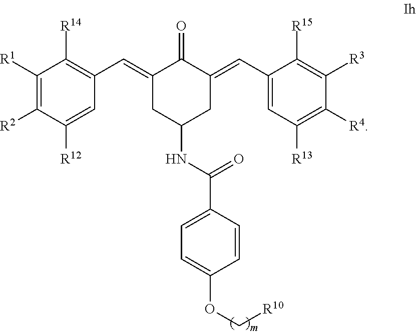

89. The compound of claim 87, wherein the compound has a structure represented by Formula IIId, or a pharmaceutically acceptable salt thereof:

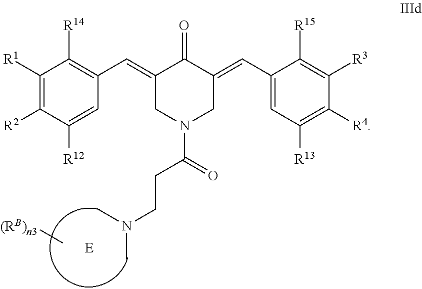

90. The compound of claim 87 or 88, wherein m is 1.

91. The compound of claim 87 or 88, wherein m is 2.

92. The compound of any one of claims 87-91, wherein R¹ is chloro.

93. The compound of any one of claims 87-91, wherein R¹ is —O(alkyl) (e.g., methoxy).

94. The compound of any one of claims 87-93, wherein R¹ is fluoro.

95. The compound of any one of claims 87-93, wherein R¹ is H.

96. The compound of any one of claims 87-95, wherein R² is fluoro.

97. The compound of any one of claims 87-95, wherein R³ is chloro.

98. The compound of any one of claims 87-95, wherein R³ is —O(alkyl) (e.g., methoxy).

99. The compound of any one of claims 87-95, wherein R³ is fluoro.

100. The compound of any one of claims 87-95, wherein R³ is H.

101. The compound of any one of claims 87-100, wherein R⁴ is fluoro.

102. The compound of any one of claims 87-88 or 90-101, wherein R¹⁰ is selected from alkyl, alkenyl, alkynyl, halo, hydroxyl, carboxyl, acyl, acetyl, ester, thioester, alkoxy, phosphoryl, amino, alkylamino, amido, carbamyl, cyano, nitro, azido, alkylthio, cycloalkyl, alkylsulfonyl, sulfonamide, carbamoylamino, cycloalkyl, aryl, heteroaryl, heterocyclylalkyl, and heterocyclyl.

103. The compound of claim 102, wherein R¹⁰ is alkylamino (e.g., ethylamino, diethylamino, or —N(CH₂)₂O(CH₃)).

104. The compound of claim 103, wherein R¹⁰ is ethylamino.

105. The compound of claim 103, wherein R¹⁰ is diethylamino.

106. The compound of claim 102, wherein R¹⁰ is heterocyclyl (e.g., 2-oxa-6-azaspiro[3.3]heptyl, piperidinyl, pyrrolidinyl, octahydrocyclopenta[c]pyrrolidinyl, or 3-azabicyclo[3.1.0]hexyl).

107. The compound of claim 106, wherein R¹⁰ is piperidinyl.

108. The compound of claim 106, wherein R¹⁰ is 2-oxa-6-azaspiro[3.3]heptyl.

109. The compound of claim 106, wherein R¹⁰ is pyrrolidinyl.

110. The compound of claim 106, wherein R¹⁰ is 3-azabicyclo[3.1.0]hexyl.

111. The compound of claim 106, wherein R¹⁰ is octahydrocyclopenta[c]pyrrolidinyl.

112. The compound of any one of claims 87-111, wherein R¹² is fluoro.

113. The compound of any one of claims 87-112, wherein R¹³ is fluoro.

114. The compound of any one of claims 87-113, wherein R¹⁴ is fluoro.

115. The compound of any one of claims 87-114, wherein R¹⁵ is fluoro.

116. The compound of any one of claims 87 or 89-101, wherein E is heterocyclyl (e.g., pyrrolidinyl, azetidinyl, octahydrocyclopenta[c]pyrrolidinyl, octahydropyrrolo[1,2-a]pyrazinyl, or 3-azabicyclo[3.1.0]hexyl).

117. The compound of any one of claims 87, 89-101, or 116, wherein R^{B} is alkyl (e.g., methyl).

118. The compound of any one of claims 87, 89-101, or 116, wherein R^{B} is halo (e.g., fluoro).

119. The compound of any one of claims 87, 89-101, or 116, wherein R^{B} is amino (e.g., dimethylamino).

120. The compound of any one of claims 87, 89-101, or 116, wherein R^{B} is alkoxy (e.g., methoxy).

121. The compound of any one of claims 87, 89-101, or 116, wherein R^{B} is sulfonyl (e.g., —S(O)₂CH₃)).

122. The compound of any one of claims 87, 89-101, or 116-121, wherein n3 is 0.

123. The compound of any one of claims 87, 89-101, or 116-121, wherein n3 is 1.

124. The compound of claim 64 or 65, wherein the compound has a structure represented by formula II or a pharmaceutically acceptable salt thereof: 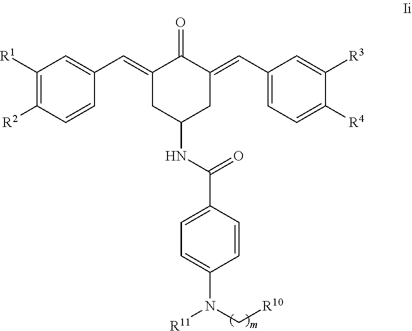
wherein R¹⁰ is selected from alkyl, alkenyl, alkynyl, halo, hydroxyl, carboxyl, acyl, acetyl, ester, thioester, alkoxy, phosphoryl, amino, alkylamino, amido, carbamyl, cyano, nitro, azido, alkylthio, cycloalkyl, alkylsulfonyl, sulfonamide, carbamoylamino, cycloalkyl, aryl, heteroaryl, and heterocyclyl;
R¹¹ is H, alkyl, or aralkyl; and
m is 1, 2, 3, 4, 5, 6, 7, 8, 9, or 10.

125. The compound of claim 64 or 65, wherein the compound has a structure represented by formula Ij, or a pharmaceutically acceptable salt thereof: 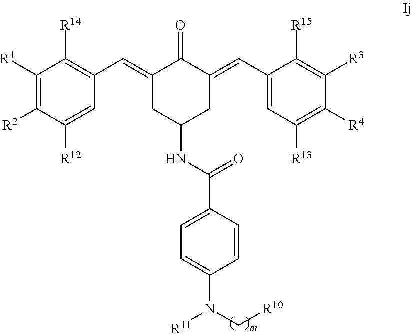
wherein R¹⁰ is selected from alkyl, alkenyl, alkynyl, halo, hydroxyl, carboxyl, acyl, acetyl, ester, thioester, alkoxy, phosphoryl, amino, alkylamino, amido, carbamyl, cyano, nitro, azido, alkylthio, cycloalkyl, alkylsulfonyl, sulfonamide, carbamoylamino, cycloalkyl, aryl, heteroaryl, and heterocyclyl;
R¹¹ is H, alkyl, or aralkyl; and
m is 1, 2, 3, 4, 5, 6, 7, 8, 9, or 10.

126. The compound of claim 124 or 125, wherein m is 1.

127. The compound of claim 124 or 125, wherein m is 2.

128. The compound of claim 124 or 125, wherein m is 3.

129. The compound of any one of claims 124-128, wherein R¹⁰ is amino.

130. The compound of claim 129, wherein R¹⁰ is NH₂, alkylamino (e.g., propylamino, pentylamino, or hexylamino), alkyloxyalkylamino (e.g., methoxypropylamino, methoxyethylamino, methoxyethylamino, or ethoxyethylamino), dialkylamino (e.g., diethylamino, dipropylamino, diisopropylamino, dibutylamino, dipentylamino, dihexylamino, or dioctylamino), dialkenylamino (e.g., diallylamino), dialkynylamino (e.g., dibutynylamino), dialkyloxyalkylamino (e.g., dimethoxyethylamino), heterocyclylalkylamino (e.g., tetrahydrofuranylmethanamino, N-methylpyrrolidinylmethanamino, or N-ethylpyrrolidinylmethanamino), dialkoxyalkylamino (e.g., diethoxyethylamino), cycloalkylamino (e.g., dicyclohexylamino), aralkylamino (e.g., dibenzylamino), (alkyl)(cycloalkyl)amino (e.g., (methyl)(cyclohexyl)amino or (ethyl)(cyclohexyl)amino).

131. The compound of any one of claims 124-128, wherein R¹⁰ is heterocyclyl (e.g., pyrrolidinyl, piperidinyl, piperazinyl, such as N-methyl piperazinyl, azepanyl, azocanyl, morpholinyl, oxazolidinonyl, and phthalimidyl).

132. The compound of any one of claims 124-128, wherein R¹⁰ is heterocyclyl (e.g., pyrrolidinyl, such as N-methyl pyrrolidinyl, piperidinyl, azetidinyl, octahydrocyclopenta[c]pyrrolidinyl, octahydropyrrolo[1,2-a]pyrazinyl, 3-azabicyclo[3.1.0]hexyl, 2-oxa-6-azaspiro[3.3]heptyl, hexahydro-5H-[1,4]dioxino[2,3-c]-pyrrolyl, piperazinyl, such as N-methyl piperazinyl, azepanyl, azocanyl, morpholinyl, oxazolidinonyl, and phthalimidyl).

133. The compound of any one of claims 124-128, wherein R¹⁰ is carbamyl (e.g., tert-butyl carbamoyl).

134. The compound of any one of claims 124-128, wherein R¹⁰ is heteroaryl (e.g., triazolyl).

135. The compound of claim 64 or 65, wherein the compound has a structure represented by formula Ik or a pharmaceutically acceptable salt thereof: 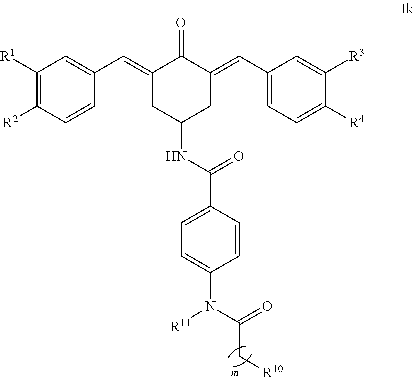
wherein R¹⁰ is selected from alkyl, alkenyl, alkynyl, halo, hydroxyl, carboxyl, acyl, acetyl, ester, thioester, alkoxy, phosphoryl, amino, alkylamino, amido, carbamyl, cyano, nitro, azido, alkylthio, cycloalkyl, alkylsulfonyl, sulfonamide, carbamoylamino, cycloalkyl, aryl, heteroaryl, and heterocyclyl;
R¹¹ is H, alkyl, or aralkyl; and
m is 0, 1, 2, 3, 4, 5, 6, 7, 8, 9, or 10.

136. The compound of claim 64 or 65, wherein the compound has a structure represented by formula II or a pharmaceutically acceptable salt thereof: 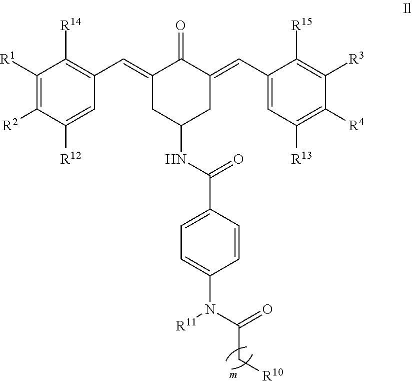
wherein R¹⁰ is selected from alkyl, alkenyl, alkynyl, halo, hydroxyl, carboxyl, acyl, acetyl, ester, thioester, alkoxy, phosphoryl, amino, alkylamino, amido, carbamyl, cyano, nitro, azido, alkylthio, cycloalkyl, alkylsulfonyl, sulfonamide, carbamoylamino, cycloalkyl, aryl, heteroaryl, and heterocyclyl;
R¹¹ is H, alkyl, or aralkyl; and
m is 0, 1, 2, 3, 4, 5, 6, 7, 8, 9, or 10.

137. The compound of claim 136, wherein m is 0.

138. The compound of claim 136, wherein m is 1.

139. The compound of any one of claims 136-138, wherein R¹⁰ is heterocyclyl (e.g., azetidine, pyrrolidinyl, pyrrolidinonyl, morpholinyl, piperidinyl, piperazinyl (such as N-methyl piperazinyl), or isoindolinyl).

140. The compound of any one of claims 136-138, wherein R¹⁰ is amino.

141. The compound of any one of claims 136-138, wherein R¹⁰ is alkylamino (e.g., diethylamino or dibutylamino), or alkoxyalkylamino (e.g., dimethoxyethylamino).

142. The compound of any one of claims 136-138, wherein R¹⁰ is heteroaryl (e.g., isoindoline).

143. The compound of any one of claims 136-142, wherein R¹⁰ is substituted with alkyl, alkenyl, alkynyl, aralkyl, halo, hydroxyl, carboxyl, acyl, acetyl, ester, thioester, alkoxy, phosphoryl, amino, alkylamino, amido, carbamyl, cyano, nitro, azido, alkylthio, cycloalkyl, alkylsulfonyl, sulfonamide, carbamoylamino, cycloalkyl, aryl, heteroaryl, and heterocyclyl.

144. The compound of any one of claims 136-142, wherein R¹⁰ is substituted with alkyl (e.g., trifluoromethyl or thiophenylethyl).

145. The compound of any one of claims 136-142, wherein R¹⁰ is substituted with aryl (e.g., phenyl).

146. The compound of any one of claims 136-142, wherein R¹⁰ is substituted with acyl (e.g., cyclopropylmethanonyl).

147. The compound of any one of claims 136-142, wherein R¹⁰ is substituted with amino (e.g., dimethylamino).

148. The compound of any one of claims 136-142, wherein R¹⁰ is substituted with heterocyclyl (e.g., benzopyranyl or pyrrolidinyl).

149. The compound of any one of claims 136-142, wherein R¹⁰ is substituted with amido (e.g., pyridinylmethylamido).

150. The compound of any one of claims 136-142, wherein R¹⁰ is substituted with ester (e.g., tert-butyl ester).

151. The compound of any one of claims 136-142, wherein R¹⁰ is substituted with aralkyl.

152. The compound of any one of claims 136-142, wherein R¹¹ is H.

153. The compound of any one of claims 136-142, wherein R¹¹ is alkyl (e.g., ethyl).

154. The compound of claim 1, wherein the compound is selected from 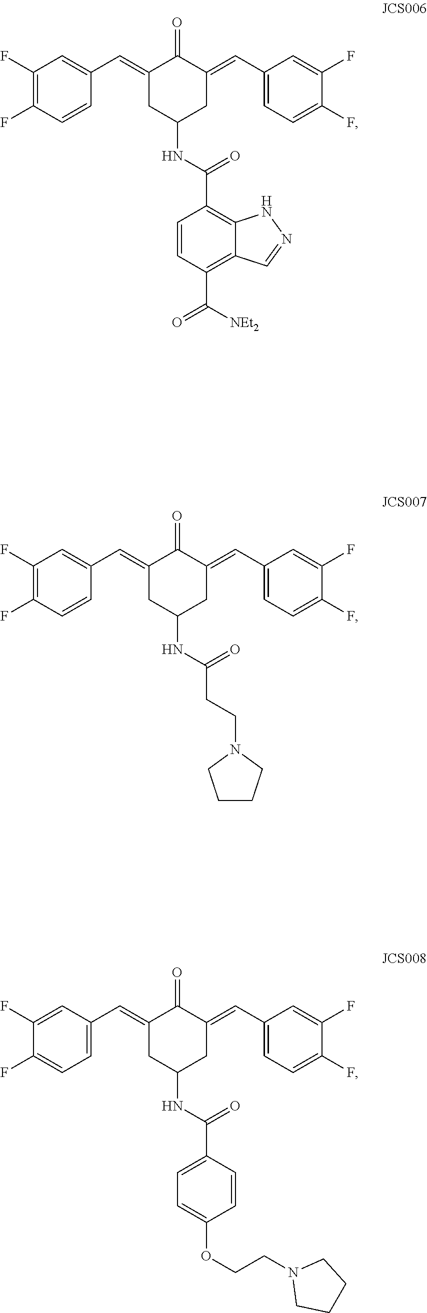 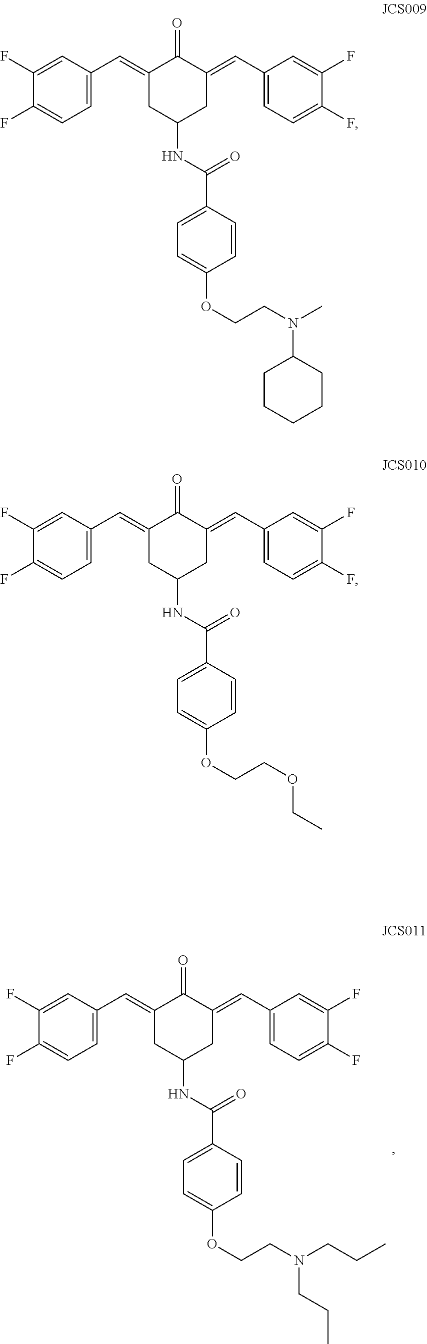 [SMILES: CN(CCOc1ccc(C(=O)NC2C/C(=C\c3ccc(F)c(F)c3)C(=O)/C(=C/c3ccc(F)c(F)c3)C2)cc1)C1CCCCC1 ; CCOCCOc1ccc(C(=O)NC2C/C(=C\c3ccc(F)c(F)c3)C(=O)/C(=C/c3ccc(F)c(F)c3)C2)cc1 ; CCCN(CCC)CCOc1ccc(C(=O)NC2C/C(=C\c3ccc(F)c(F)c3)C(=O)/C(=C/c3ccc(F)c(F)c3)C2)cc1] 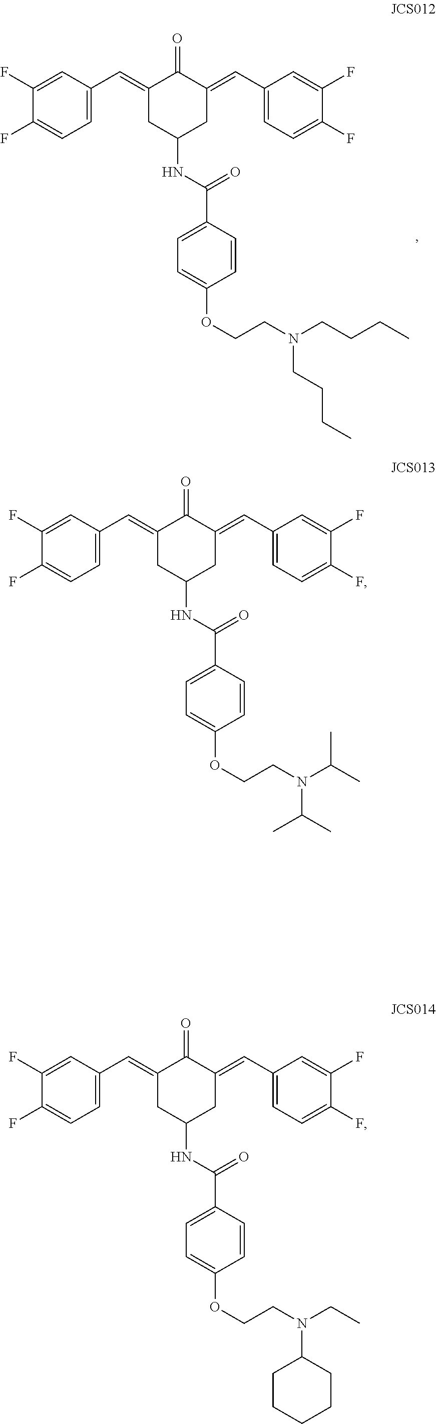 [SMILES: CCCCN(CCCC)CCOc1ccc(C(=O)NC2C/C(=C\c3ccc(F)c(F)c3)C(=O)/C(=C/c3ccc(F)c(F)c3)C2)cc1 ; CC(C)N(CCOc1ccc(C(=O)NC2C/C(=C\c3ccc(F)c(F)c3)C(=O)/C(=C/c3ccc(F)c(F)c3)C2)cc1)C(C)C ; CCN(CCOc1ccc(C(=O)NC2C/C(=C\c3ccc(F)c(F)c3)C(=O)/C(=C/c3ccc(F)c(F)c3)C2)cc1)C1CCCCC1] 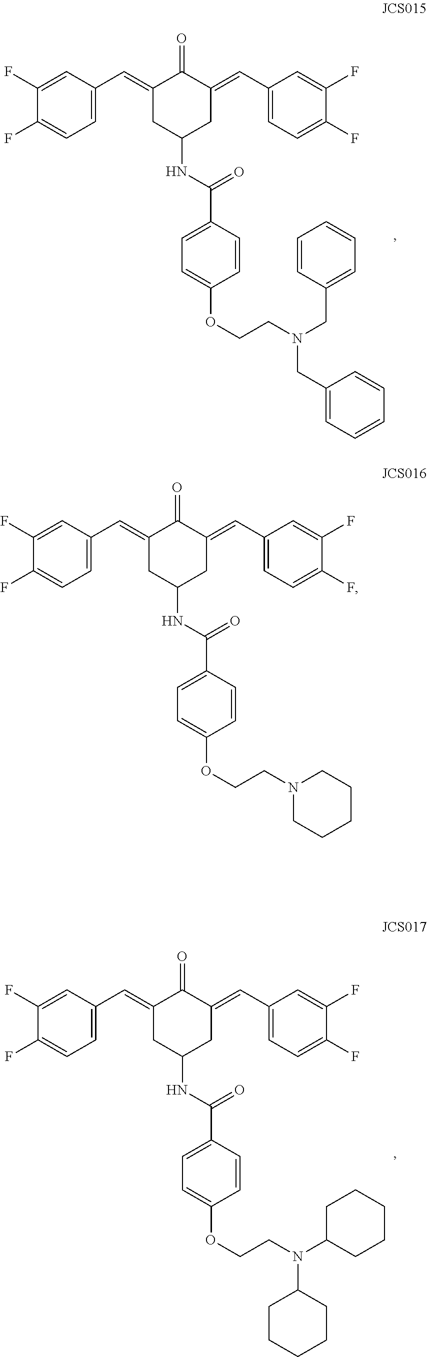 [SMILES: O=C1/C(=C/c2ccc(F)c(F)c2)CC(NC(=O)c2ccc(OCCN(Cc3ccccc3)Cc3ccccc3)cc2)C/C1=C\c1ccc(F)c(F)c1 ; O=C1/C(=C/c2ccc(F)c(F)c2)CC(NC(=O)c2ccc(OCCN3CCCCC3)cc2)C/C1=C\c1ccc(F)c(F)c1 ; O=C1/C(=C/c2ccc(F)c(F)c2)CC(NC(=O)c2ccc(OCCN(C3CCCCC3)C3CCCCC3)cc2)C/C1=C\c1ccc(F)c(F)c1] 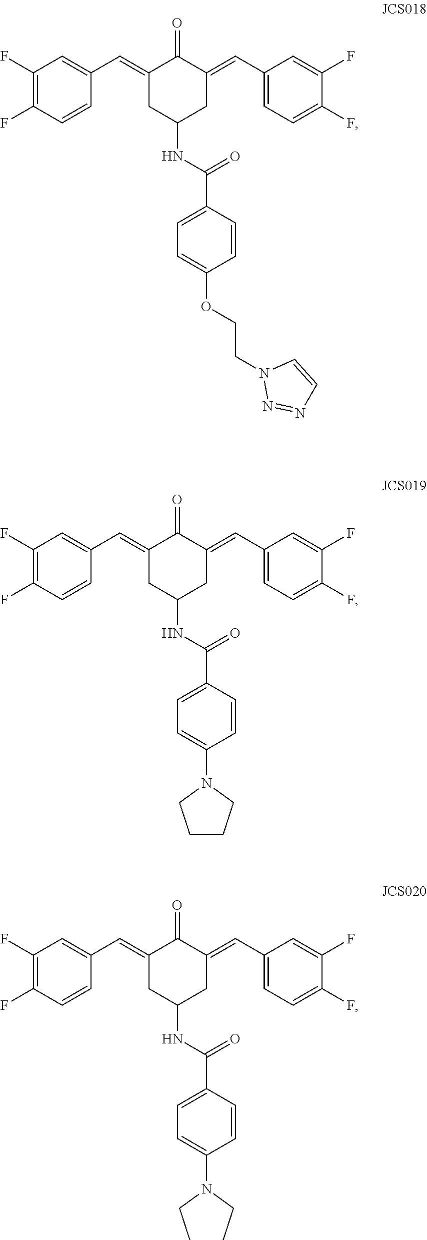 [SMILES: O=C1/C(=C/c2ccc(F)c(F)c2)CC(NC(=O)c2ccc(OCCn3ccnn3)cc2)C/C1=C\c1ccc(F)c(F)c1 ; O=C1/C(=C/c2ccc(F)c(F)c2)CC(NC(=O)c2ccc(N3CCCC3)cc2)C/C1=C\c1ccc(F)c(F)c1 ; CCN(CC)c1ccc(C(=O)NC2C/C(=C\c3ccc(F)c(F)c3)C(=O)/C(=C/c3ccc(F)c(F)c3)C2)cc1] 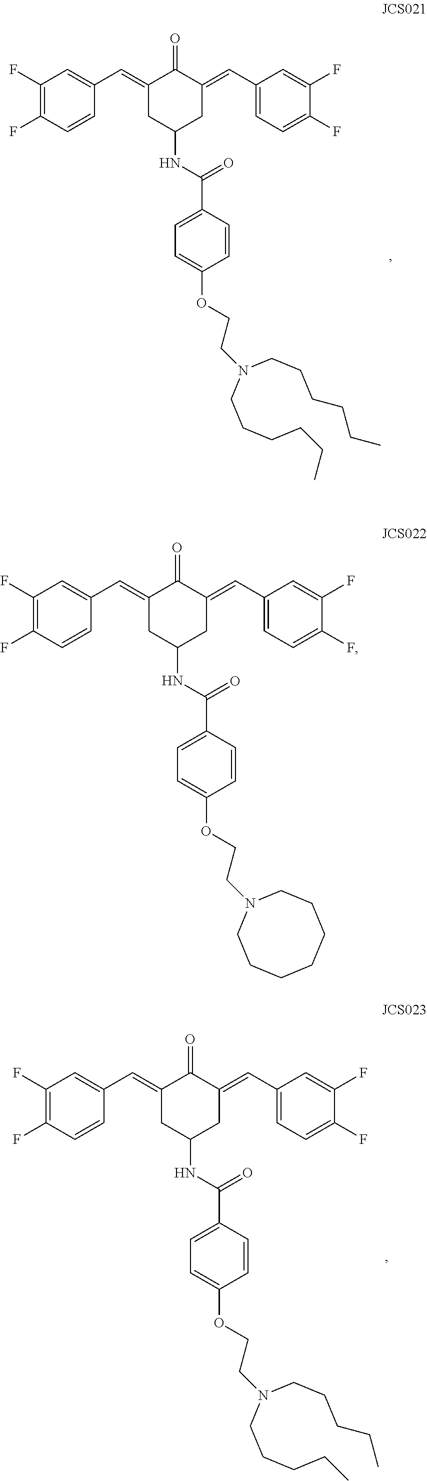 [SMILES: CCCCCCN(CCCCCC)CCOc1ccc(C(=O)NC2C/C(=C\c3ccc(F)c(F)c3)C(=O)/C(=C/c3ccc(F)c(F)c3)C2)cc1 ; O=C1/C(=C/c2ccc(F)c(F)c2)CC(NC(=O)c2ccc(OCCN3CCCCCCC3)cc2)C/C1=C\c1ccc(F)c(F)c1 ; CCCCCN(CCCCC)CCOc1ccc(C(=O)NC2C/C(=C\c3ccc(F)c(F)c3)C(=O)/C(=C/c3ccc(F)c(F)c3)C2)cc1] 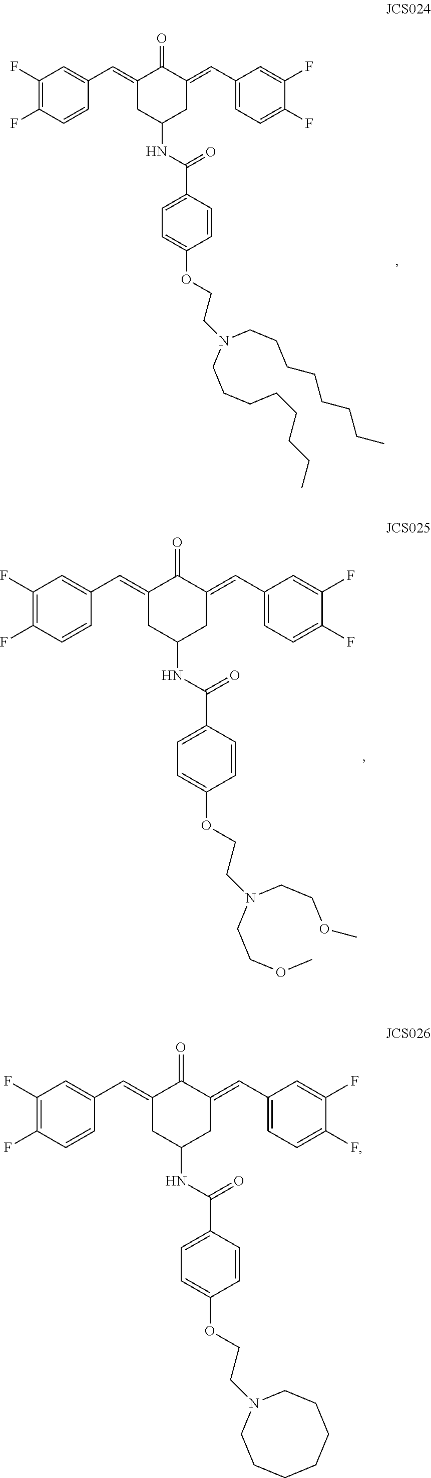 [SMILES: CCCCCCCCN(CCCCCCCC)CCOc1ccc(C(=O)NC2C/C(=C\c3ccc(F)c(F)c3)C(=O)/C(=C/c3ccc(F)c(F)c3)C2)cc1 ; COCCN(CCOC)CCOc1ccc(C(=O)NC2C/C(=C\c3ccc(F)c(F)c3)C(=O)/C(=C/c3ccc(F)c(F)c3)C2)cc1 ; O=C1/C(=C/c2ccc(F)c(F)c2)CC(NC(=O)c2ccc(OCCN3CCCCCCC3)cc2)C/C1=C\c1ccc(F)c(F)c1] 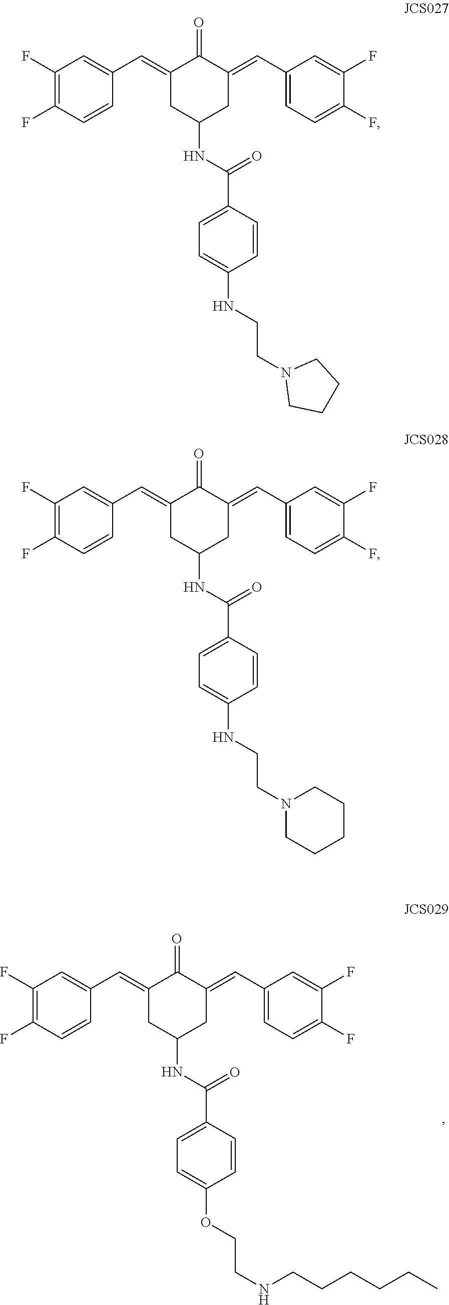 [SMILES: O=C1/C(=C/c2ccc(F)c(F)c2)CC(NC(=O)c2ccc(NCCN3CCCC3)cc2)C/C1=C\c1ccc(F)c(F)c1 ; O=C1/C(=C/c2ccc(F)c(F)c2)CC(NC(=O)c2ccc(NCCN3CCCCC3)cc2)C/C1=C\c1ccc(F)c(F)c1 ; CCCCCCNCCOc1ccc(C(=O)NC2C/C(=C\c3ccc(F)c(F)c3)C(=O)/C(=C/c3ccc(F)c(F)c3)C2)cc1] 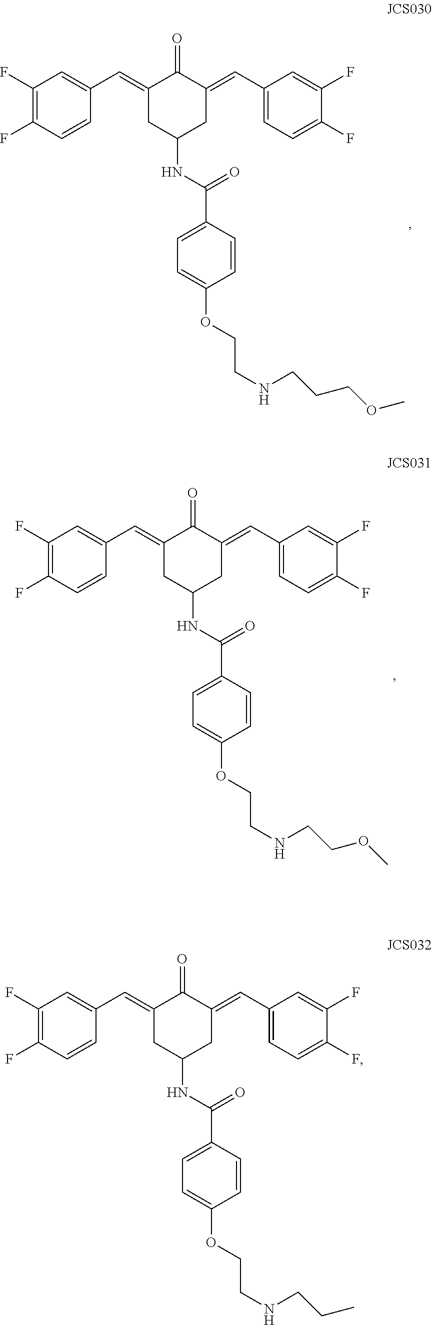 [SMILES: COCCCNCCOc1ccc(C(=O)NC2C/C(=C\c3ccc(F)c(F)c3)C(=O)/C(=C/c3ccc(F)c(F)c3)C2)cc1 ; COCCNCCOc1ccc(C(=O)NC2C/C(=C\c3ccc(F)c(F)c3)C(=O)/C(=C/c3ccc(F)c(F)c3)C2)cc1 ; CCCNCCOc1ccc(C(=O)NC2C/C(=C\c3ccc(F)c(F)c3)C(=O)/C(=C/c3ccc(F)c(F)c3)C2)cc1] 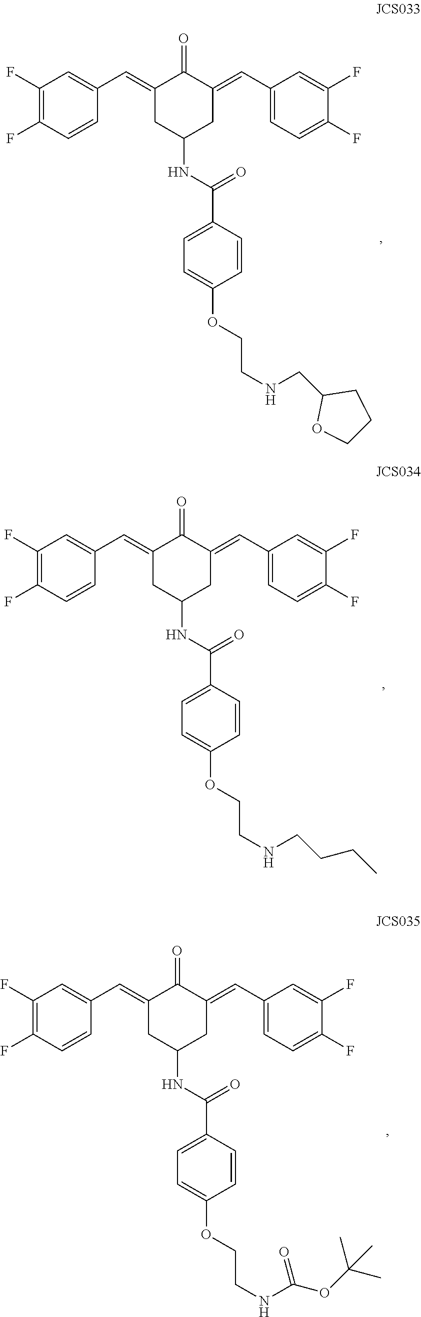 [SMILES: O=C1/C(=C/c2ccc(F)c(F)c2)CC(NC(=O)c2ccc(OCCNCC3CCCO3)cc2)C/C1=C\c1ccc(F)c(F)c1 ; CCCCNCCOc1ccc(C(=O)NC2C/C(=C\c3ccc(F)c(F)c3)C(=O)/C(=C/c3ccc(F)c(F)c3)C2)cc1 ; CC(C)(C)OC(=O)NCCOc1ccc(C(=O)NC2C/C(=C\c3ccc(F)c(F)c3)C(=O)/C(=C/c3ccc(F)c(F)c3)C2)cc1] 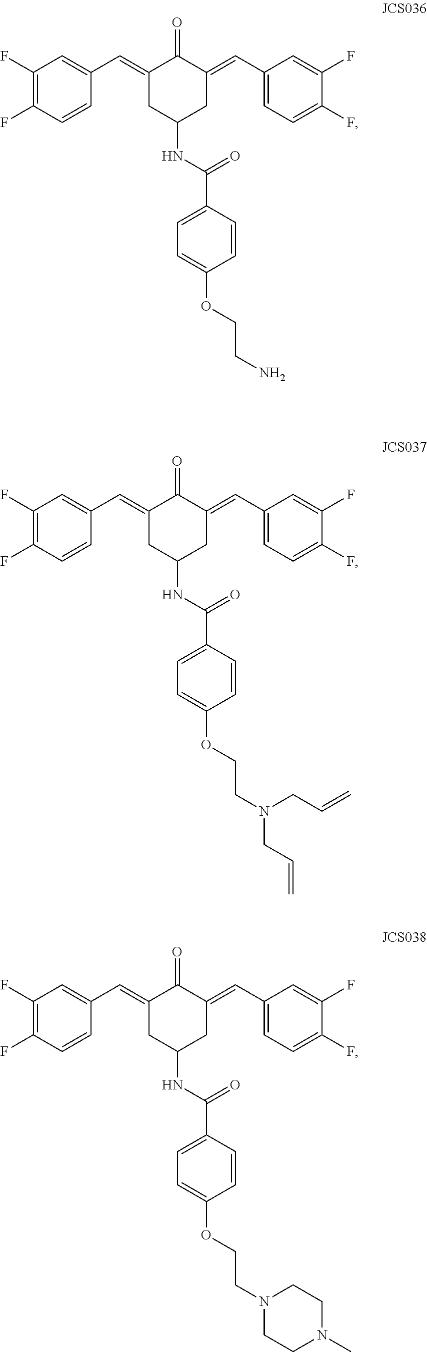 [SMILES: NCCOc1ccc(C(=O)NC2C/C(=C\c3ccc(F)c(F)c3)C(=O)/C(=C/c3ccc(F)c(F)c3)C2)cc1 ; C=CCN(CC=C)CCOc1ccc(C(=O)NC2C/C(=C\c3ccc(F)c(F)c3)C(=O)/C(=C/c3ccc(F)c(F)c3)C2)cc1 ; CN1CCN(CCOc2ccc(C(=O)NC3C/C(=C\c4ccc(F)c(F)c4)C(=O)/C(=C/c4ccc(F)c(F)c4)C3)cc2)CC1] 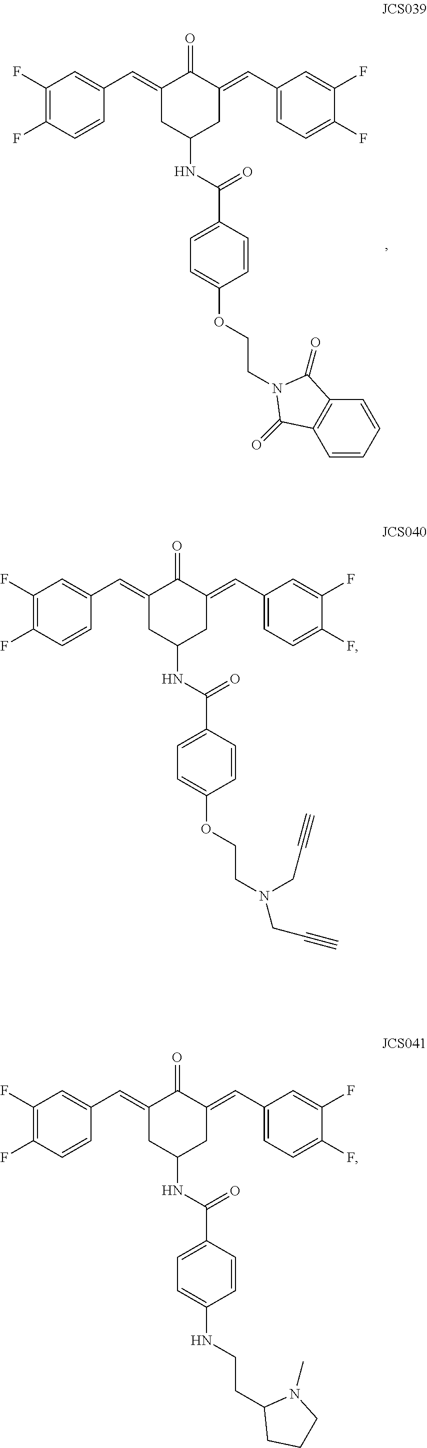 [SMILES: O=C1/C(=C/c2ccc(F)c(F)c2)CC(NC(=O)c2ccc(OCCN3C(=O)c4ccccc4C3=O)cc2)C/C1=C\c1ccc(F)c(F)c1 ; C#CCN(CC#C)CCOc1ccc(C(=O)NC2C/C(=C\c3ccc(F)c(F)c3)C(=O)/C(=C/c3ccc(F)c(F)c3)C2)cc1 ; CN1CCCC1CCNc1ccc(C(=O)NC2C/C(=C\c3ccc(F)c(F)c3)C(=O)/C(=C/c3ccc(F)c(F)c3)C2)cc1] 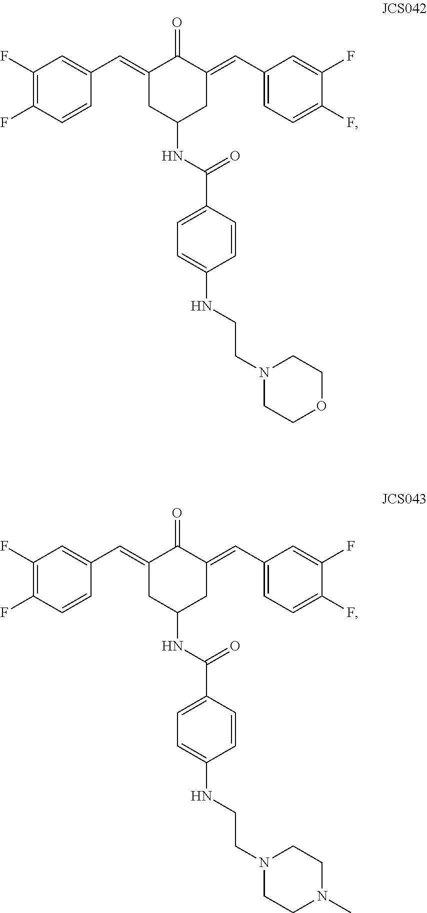 [SMILES: O=C1/C(=C/c2ccc(F)c(F)c2)CC(NC(=O)c2ccc(NCCN3CCOCC3)cc2)C/C1=C\c1ccc(F)c(F)c1 ; CN1CCN(CCNc2ccc(C(=O)NC3C/C(=C\c4ccc(F)c(F)c4)C(=O)/C(=C/c4ccc(F)c(F)c4)C3)cc2)CC1] 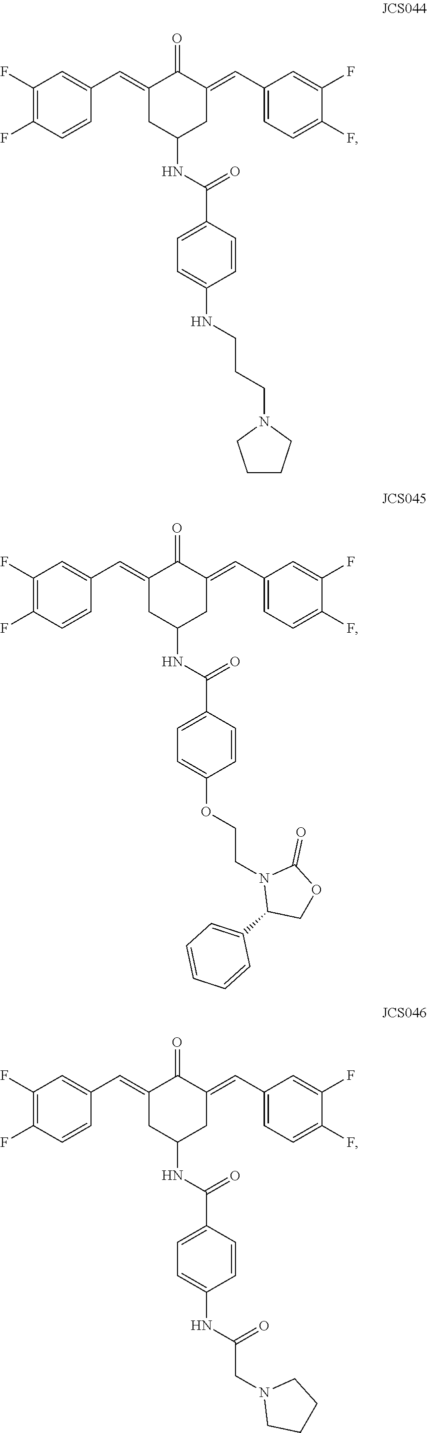 [SMILES: O=C1/C(=C/c2ccc(F)c(F)c2)CC(NC(=O)c2ccc(NCCCN3CCCC3)cc2)C/C1=C\c1ccc(F)c(F)c1 ; O=C1/C(=C/c2ccc(F)c(F)c2)CC(NC(=O)c2ccc(OCCN3C(=O)OC[C@@H]3c3ccccc3)cc2)C/C1=C\c1ccc(F)c(F)c1 ; O=C(CN1CCCC1)Nc1ccc(C(=O)NC2C/C(=C\c3ccc(F)c(F)c3)C(=O)/C(=C/c3ccc(F)c(F)c3)C2)cc1] 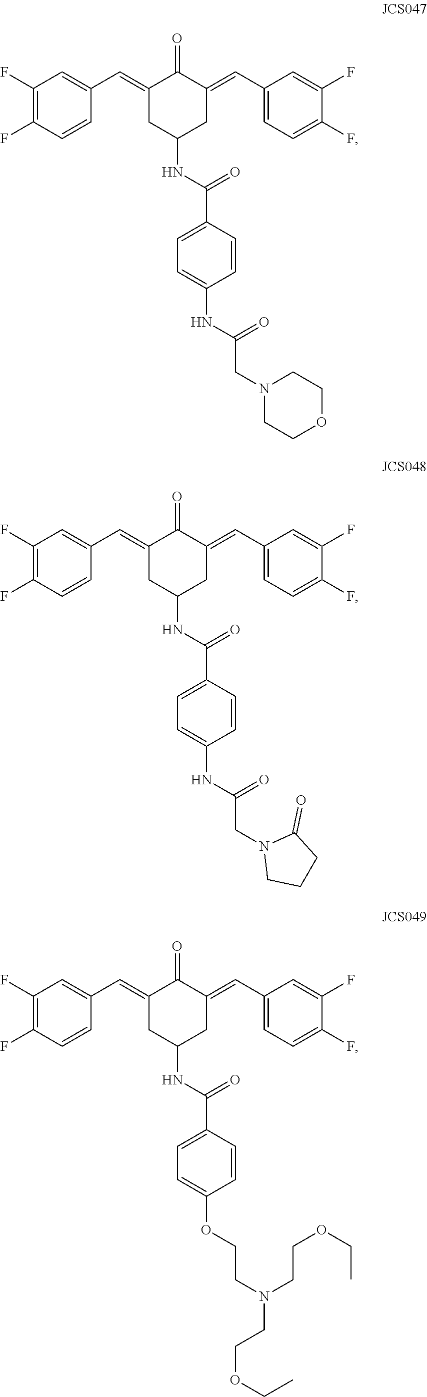 [SMILES: O=C(CN1CCOCC1)Nc1ccc(C(=O)NC2C/C(=C\c3ccc(F)c(F)c3)C(=O)/C(=C/c3ccc(F)c(F)c3)C2)cc1 ; O=C(CN1CCCC1=O)Nc1ccc(C(=O)NC2C/C(=C\c3ccc(F)c(F)c3)C(=O)/C(=C/c3ccc(F)c(F)c3)C2)cc1 ; CCOCCN(CCOCC)CCOc1ccc(C(=O)NC2C/C(=C\c3ccc(F)c(F)c3)C(=O)/C(=C/c3ccc(F)c(F)c3)C2)cc1] 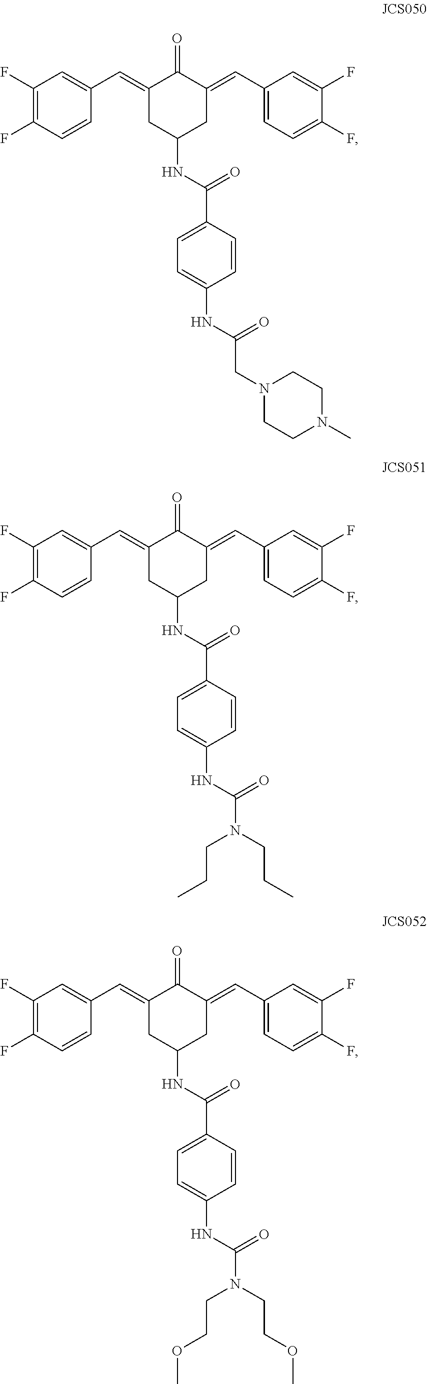 [SMILES: CN1CCN(CC(=O)Nc2ccc(C(=O)NC3C/C(=C\c4ccc(F)c(F)c4)C(=O)/C(=C/c4ccc(F)c(F)c4)C3)cc2)CC1 ; CCCN(CCC)C(=O)Nc1ccc(C(=O)NC2C/C(=C\c3ccc(F)c(F)c3)C(=O)/C(=C/c3ccc(F)c(F)c3)C2)cc1 ; COCCN(CCOC)C(=O)Nc1ccc(C(=O)NC2C/C(=C\c3ccc(F)c(F)c3)C(=O)/C(=C/c3ccc(F)c(F)c3)C2)cc1] 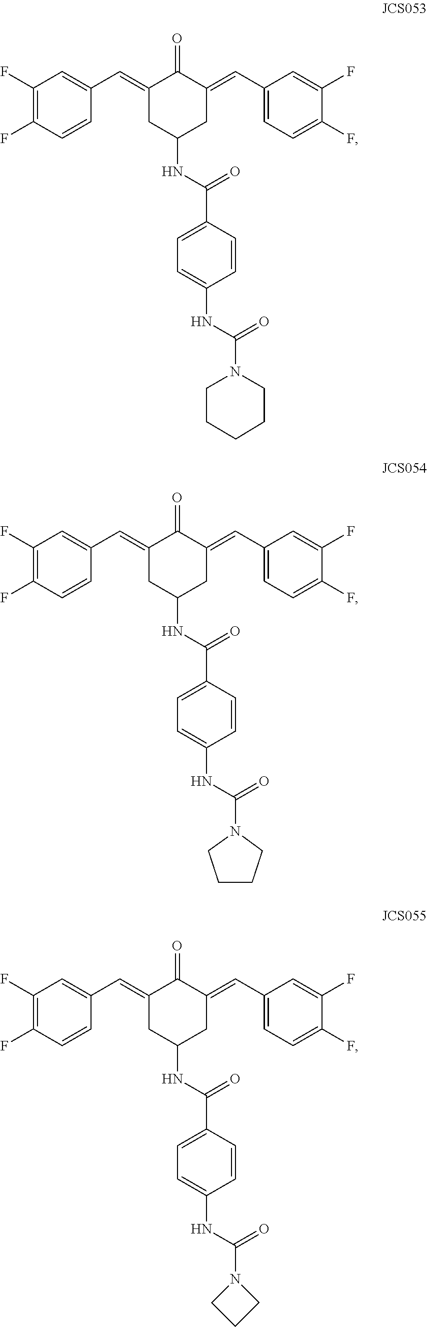 [SMILES: O=C1/C(=C/c2ccc(F)c(F)c2)CC(NC(=O)c2ccc(NC(=O)N3CCCCC3)cc2)C/C1=C\c1ccc(F)c(F)c1 ; O=C1/C(=C/c2ccc(F)c(F)c2)CC(NC(=O)c2ccc(NC(=O)N3CCCC3)cc2)C/C1=C\c1ccc(F)c(F)c1 ; O=C1/C(=C/c2ccc(F)c(F)c2)CC(NC(=O)c2ccc(NC(=O)N3CCC3)cc2)C/C1=C\c1ccc(F)c(F)c1] 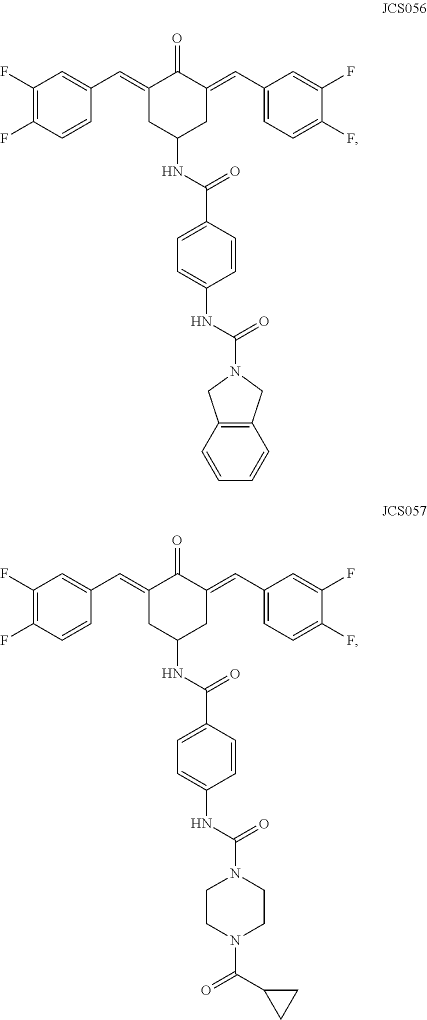 [SMILES: O=C1/C(=C/c2ccc(F)c(F)c2)CC(NC(=O)c2ccc(NC(=O)N3Cc4ccccc4C3)cc2)C/C1=C\c1ccc(F)c(F)c1 ; O=C1/C(=C/c2ccc(F)c(F)c2)CC(NC(=O)c2ccc(NC(=O)N3CCN(C(=O)C4CC4)CC3)cc2)C/C1=C\c1ccc(F)c(F)c1] 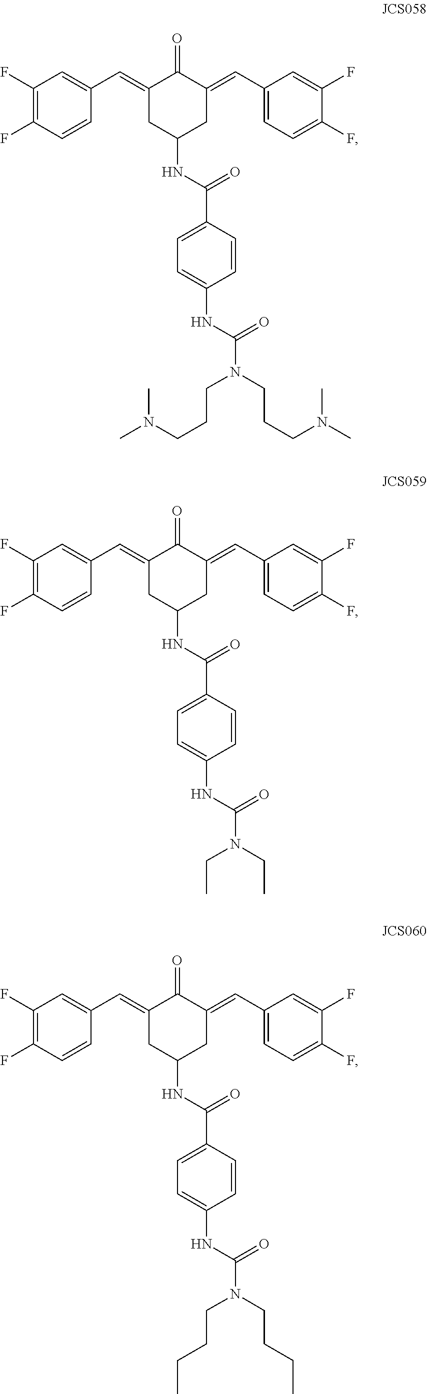 [SMILES: CN(C)CCCN(CCCN(C)C)C(=O)Nc1ccc(C(=O)NC2C/C(=C\c3ccc(F)c(F)c3)C(=O)/C(=C/c3ccc(F)c(F)c3)C2)cc1 ; CCN(CC)C(=O)Nc1ccc(C(=O)NC2C/C(=C\c3ccc(F)c(F)c3)C(=O)/C(=C/c3ccc(F)c(F)c3)C2)cc1 ; CCCCN(CCCC)C(=O)Nc1ccc(C(=O)NC2C/C(=C\c3ccc(F)c(F)c3)C(=O)/C(=C/c3ccc(F)c(F)c3)C2)cc1] 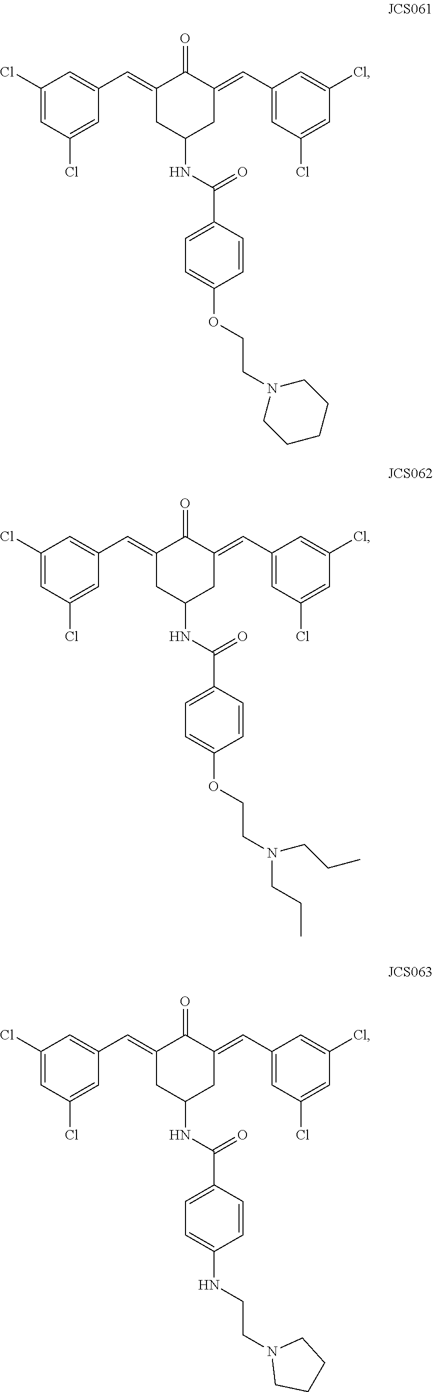 [SMILES: O=C1/C(=C/c2cc(Cl)cc(Cl)c2)CC(NC(=O)c2ccc(OCCN3CCCCC3)cc2)C/C1=C\c1cc(Cl)cc(Cl)c1 ; CCCN(CCC)CCOc1ccc(C(=O)NC2C/C(=C\c3cc(Cl)cc(Cl)c3)C(=O)/C(=C/c3cc(Cl)cc(Cl)c3)C2)cc1 ; O=C1/C(=C/c2cc(Cl)cc(Cl)c2)CC(NC(=O)c2ccc(NCCN3CCCC3)cc2)C/C1=C\c1cc(Cl)cc(Cl)c1] 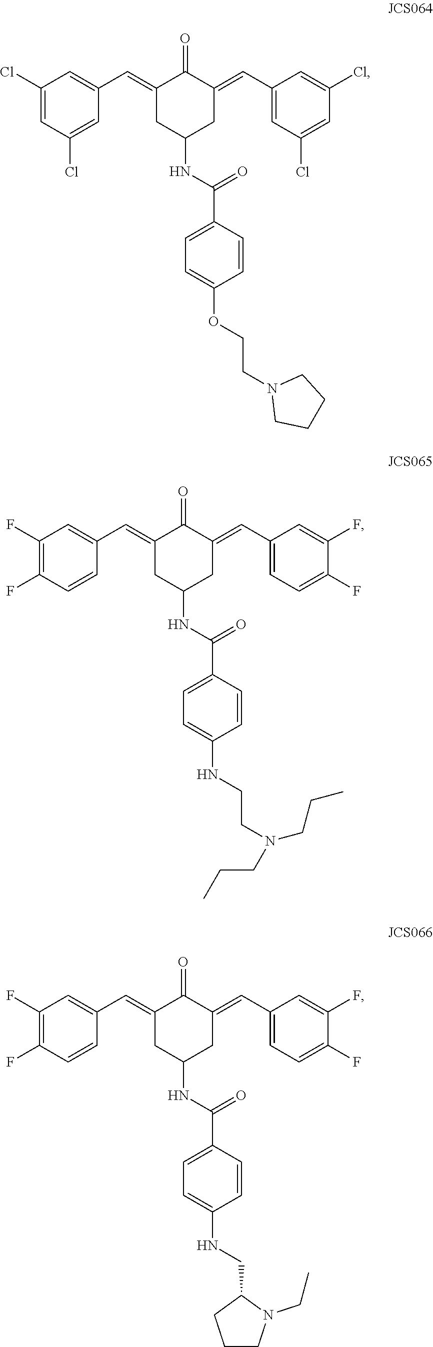 [SMILES: O=C1/C(=C/c2cc(Cl)cc(Cl)c2)CC(NC(=O)c2ccc(OCCN3CCCC3)cc2)C/C1=C\c1cc(Cl)cc(Cl)c1 ; CCCN(CCC)CCNc1ccc(C(=O)NC2C/C(=C\c3ccc(F)c(F)c3)C(=O)/C(=C/c3ccc(F)c(F)c3)C2)cc1 ; CCN1CCC[C@@H]1CNc1ccc(C(=O)NC2C/C(=C\c3ccc(F)c(F)c3)C(=O)/C(=C/c3ccc(F)c(F)c3)C2)cc1] 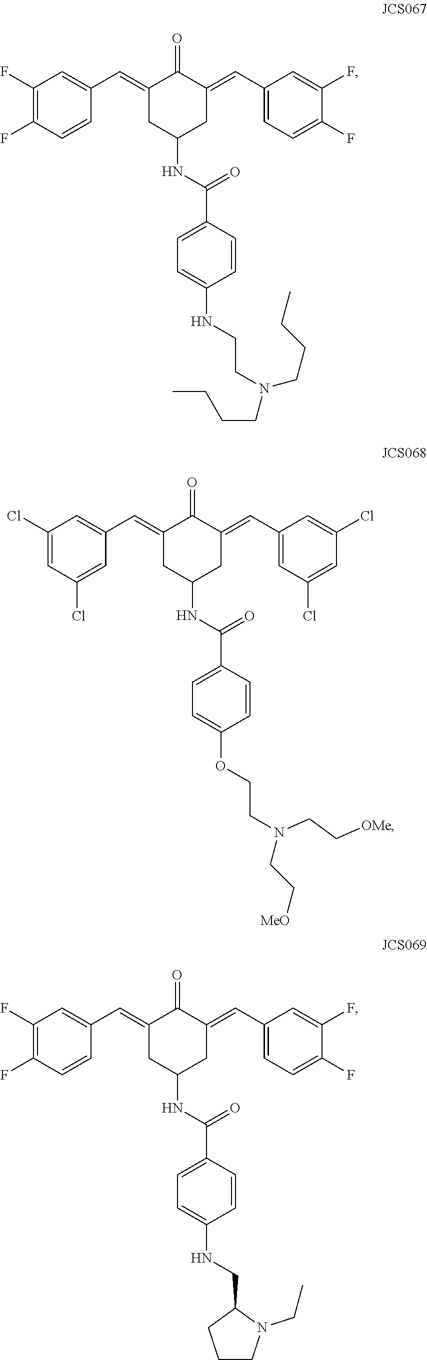 [SMILES: CCCCN(CCCC)CCNc1ccc(C(=O)NC2C/C(=C\c3ccc(F)c(F)c3)C(=O)/C(=C/c3ccc(F)c(F)c3)C2)cc1 ; COCCN(CCOC)CCOc1ccc(C(=O)NC2C/C(=C\c3cc(Cl)cc(Cl)c3)C(=O)/C(=C/c3cc(Cl)cc(Cl)c3)C2)cc1 ; CCN1CCC[C@H]1CNc1ccc(C(=O)NC2C/C(=C\c3ccc(F)c(F)c3)C(=O)/C(=C/c3ccc(F)c(F)c3)C2)cc1] 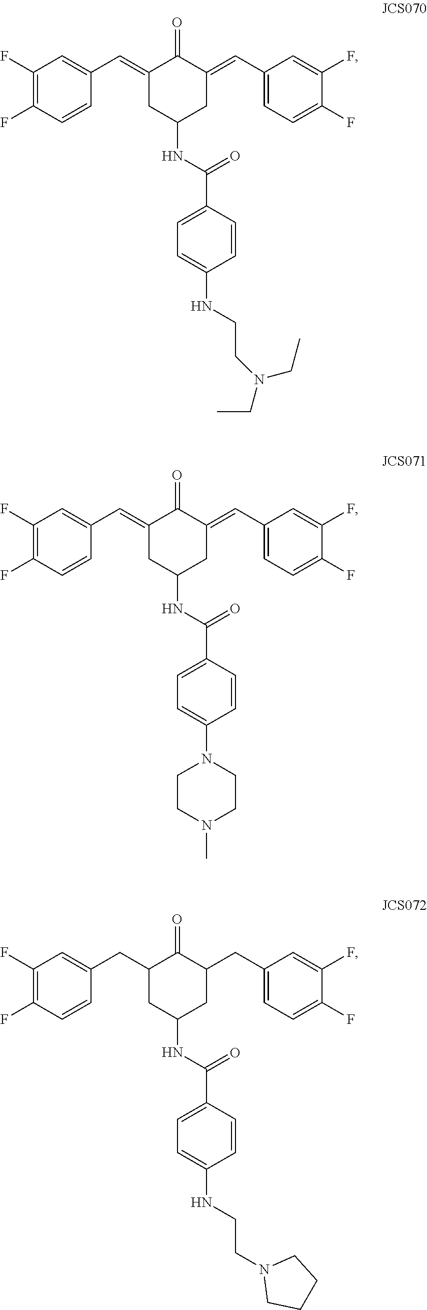 [SMILES: CCN(CC)CCNc1ccc(C(=O)NC2C/C(=C\c3ccc(F)c(F)c3)C(=O)/C(=C/c3ccc(F)c(F)c3)C2)cc1 ; CN1CCN(c2ccc(C(=O)NC3C/C(=C\c4ccc(F)c(F)c4)C(=O)/C(=C/c4ccc(F)c(F)c4)C3)cc2)CC1 ; O=C(NC1CC(Cc2ccc(F)c(F)c2)C(=O)C(Cc2ccc(F)c(F)c2)C1)c1ccc(NCCN2CCCC2)cc1] 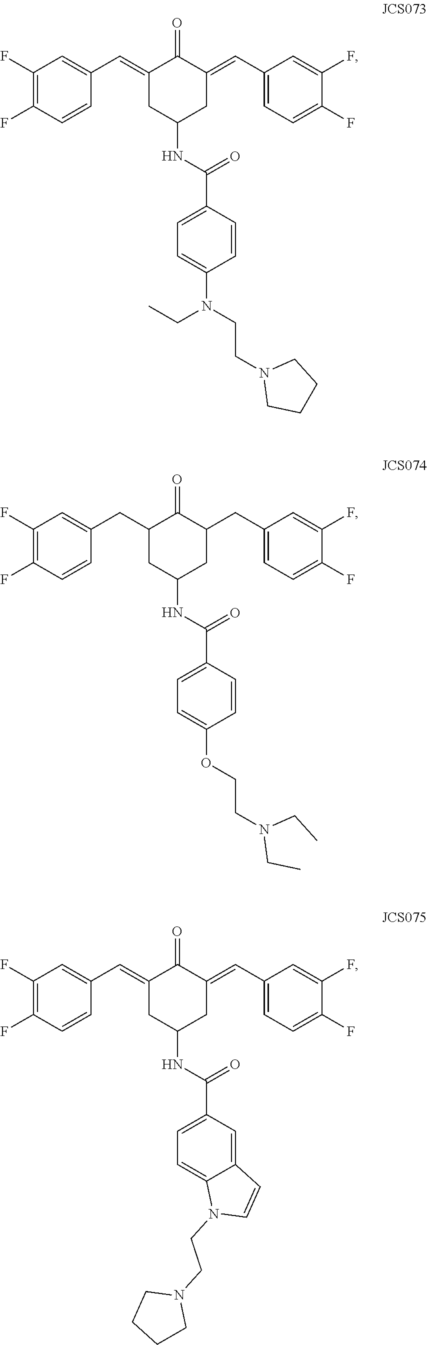 [SMILES: CCN(CCN1CCCC1)c1ccc(C(=O)NC2C/C(=C\c3ccc(F)c(F)c3)C(=O)/C(=C/c3ccc(F)c(F)c3)C2)cc1 ; CCN(CC)CCOc1ccc(C(=O)NC2CC(Cc3ccc(F)c(F)c3)C(=O)C(Cc3ccc(F)c(F)c3)C2)cc1 ; O=C1/C(=C/c2ccc(F)c(F)c2)CC(NC(=O)c2ccc3c(ccn3CCN3CCCC3)c2)C/C1=C\c1ccc(F)c(F)c1] 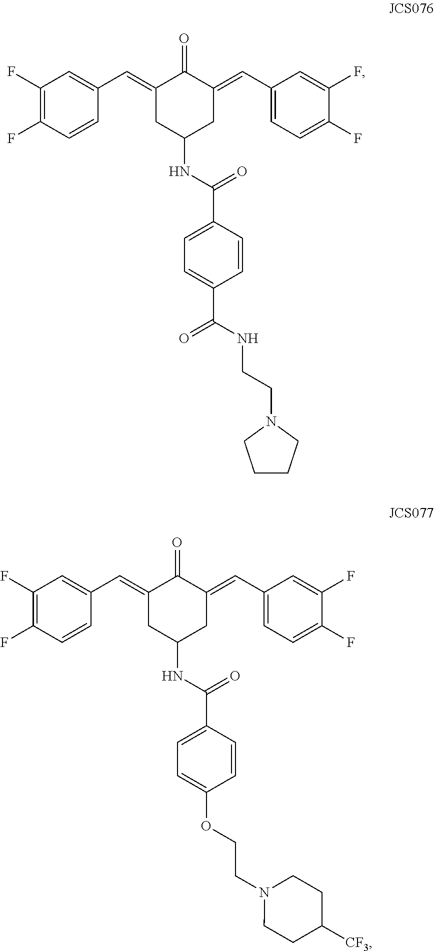 [SMILES: O=C1/C(=C/c2ccc(F)c(F)c2)CC(NC(=O)c2ccc(C(=O)NCCN3CCCC3)cc2)C/C1=C\c1ccc(F)c(F)c1 ; O=C1/C(=C/c2ccc(F)c(F)c2)CC(NC(=O)c2ccc(OCCN3CCC(C(F)(F)F)CC3)cc2)C/C1=C\c1ccc(F)c(F)c1] 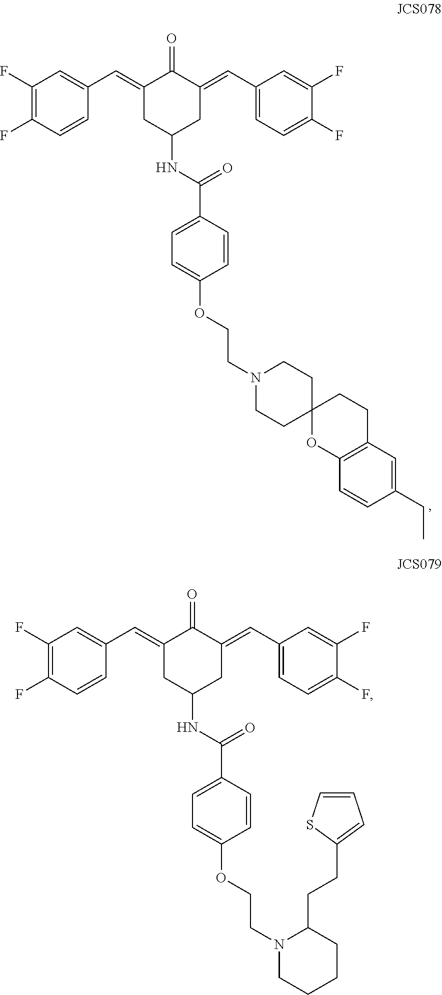 [SMILES: CCc1ccc2c(c1)CCC1(CCN(CCOc3ccc(C(=O)NC4C/C(=C\c5ccc(F)c(F)c5)C(=O)/C(=C/c5ccc(F)c(F)c5)C4)cc3)CC1)O2 ; O=C1/C(=C/c2ccc(F)c(F)c2)CC(NC(=O)c2ccc(OCCN3CCCCC3CCc3cccs3)cc2)C/C1=C\c1ccc(F)c(F)c1] 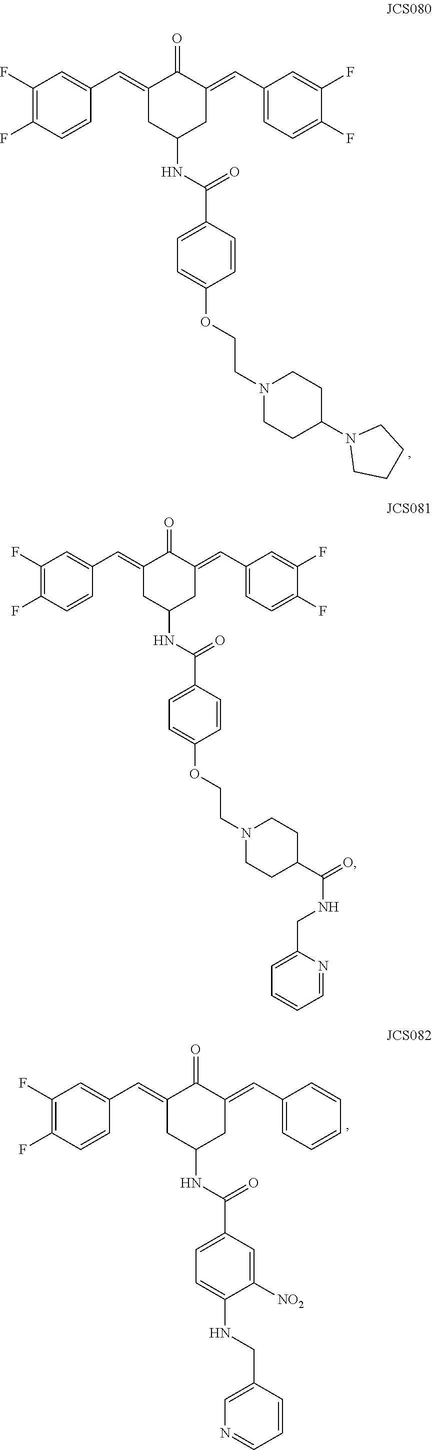 [SMILES: O=C1/C(=C/c2ccc(F)c(F)c2)CC(NC(=O)c2ccc(OCCN3CCC(N4CCCC4)CC3)cc2)C/C1=C\c1ccc(F)c(F)c1 ; O=C1/C(=C/c2ccc(F)c(F)c2)CC(NC(=O)c2ccc(OCCN3CCC(C(=O)NCc4ccccn4)CC3)cc2)C/C1=C\c1ccc(F)c(F)c1 ; O=C1/C(=C/c2ccccc2)CC(NC(=O)c2ccc(NCc3cccnc3)c([N+](=O)[O-])c2)C/C1=C\c1ccc(F)c(F)c1] 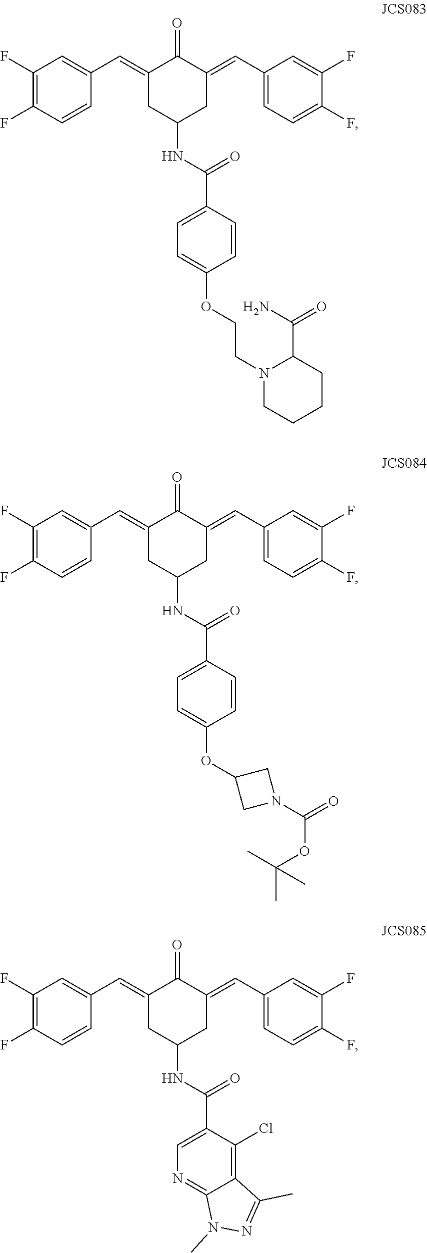 [SMILES: NC(=O)C1CCCCN1CCOc1ccc(C(=O)NC2C/C(=C\c3ccc(F)c(F)c3)C(=O)/C(=C/c3ccc(F)c(F)c3)C2)cc1 ; CC(C)(C)OC(=O)N1CC(Oc2ccc(C(=O)NC3C/C(=C\c4ccc(F)c(F)c4)C(=O)/C(=C/c4ccc(F)c(F)c4)C3)cc2)C1 ; Cc1nn(C)c2ncc(C(=O)NC3C/C(=C\c4ccc(F)c(F)c4)C(=O)/C(=C/c4ccc(F)c(F)c4)C3)c(Cl)c12] 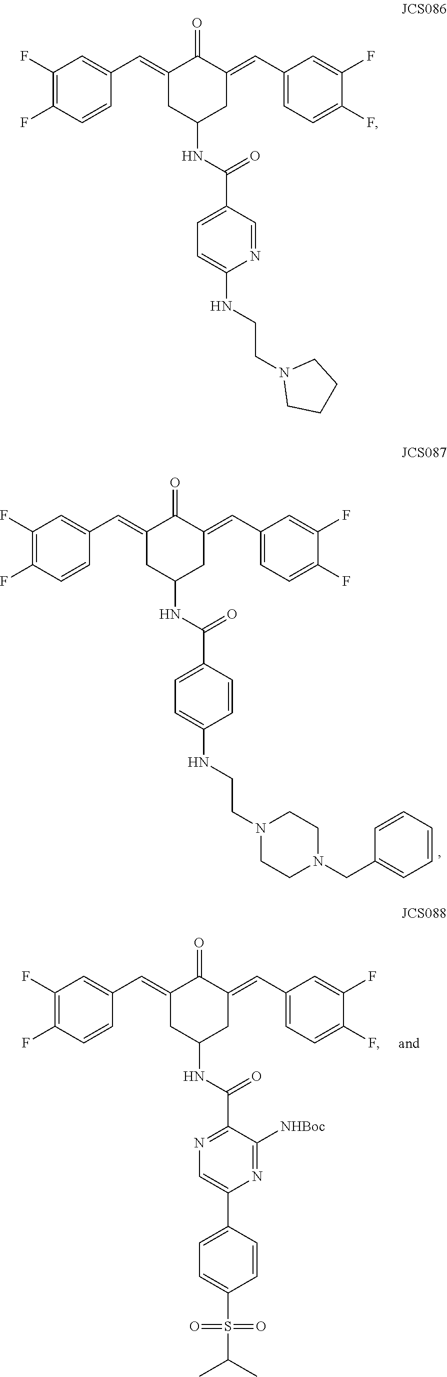 [SMILES: O=C1/C(=C/c2ccc(F)c(F)c2)CC(NC(=O)c2ccc(NCCN3CCCC3)nc2)C/C1=C\c1ccc(F)c(F)c1 ; O=C1/C(=C/c2ccc(F)c(F)c2)CC(NC(=O)c2ccc(NCCN3CCN(Cc4ccccc4)CC3)cc2)C/C1=C\c1ccc(F)c(F)c1 ; CC(C)S(=O)(=O)c1ccc(-c2cnc(C(=O)NC3C/C(=C\c4ccc(F)c(F)c4)C(=O)/C(=C/c4ccc(F)c(F)c4)C3)c(NC(=O)OC(C)(C)C)n2)cc1] 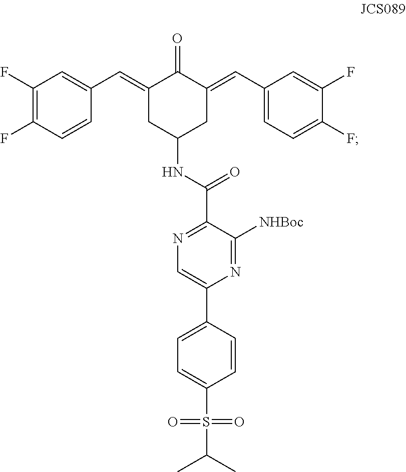 [SMILES: CC(C)S(=O)(=O)c1ccc(-c2cnc(C(=O)NC3C/C(=C\c4ccc(F)c(F)c4)C(=O)/C(=C/c4ccc(F)c(F)c4)C3)c(NC(=O)OC(C)(C)C)n2)cc1]
or a pharmaceutically acceptable salt thereof.

155. The compound of claim 1, wherein the compound is selected from 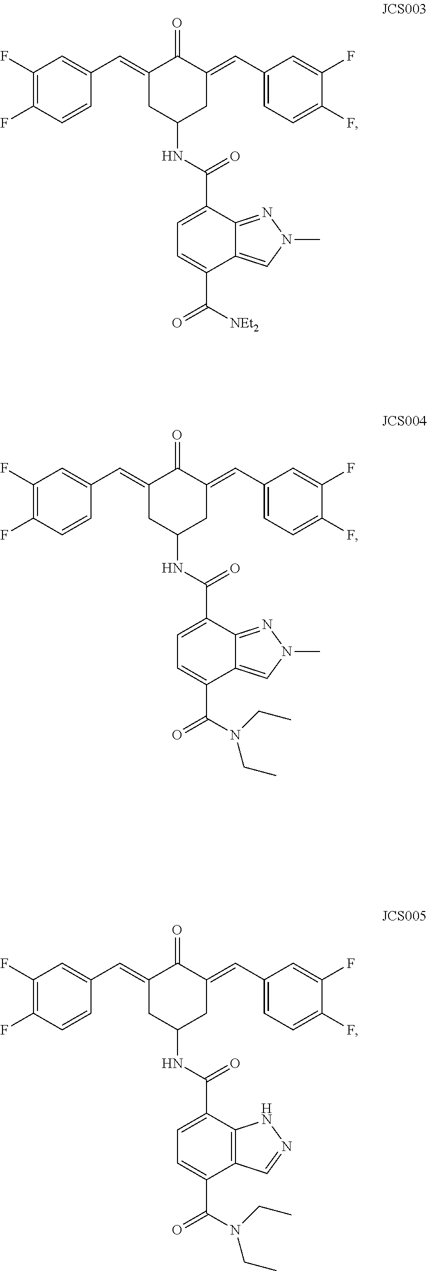 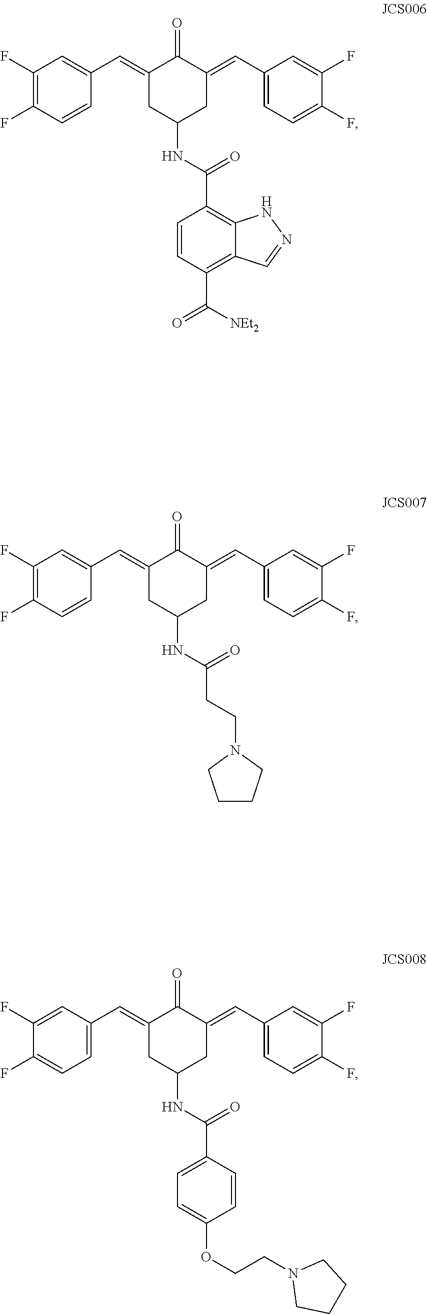 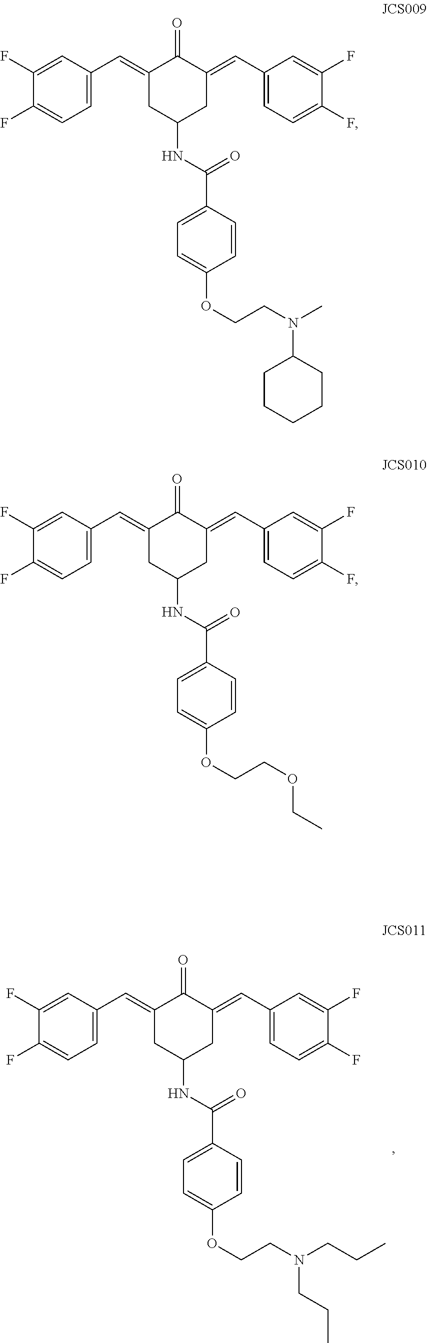 [SMILES: CN(CCOc1ccc(C(=O)NC2C/C(=C\c3ccc(F)c(F)c3)C(=O)/C(=C/c3ccc(F)c(F)c3)C2)cc1)C1CCCCC1 ; CCOCCOc1ccc(C(=O)NC2C/C(=C\c3ccc(F)c(F)c3)C(=O)/C(=C/c3ccc(F)c(F)c3)C2)cc1 ; CCCN(CCC)CCOc1ccc(C(=O)NC2C/C(=C\c3ccc(F)c(F)c3)C(=O)/C(=C/c3ccc(F)c(F)c3)C2)cc1] 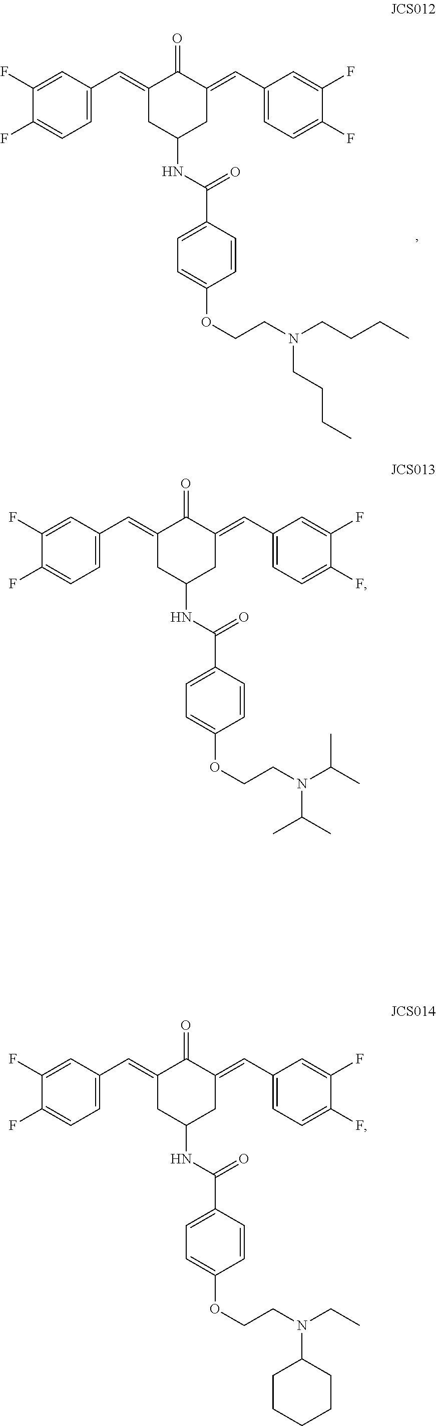 [SMILES: CCCCN(CCCC)CCOc1ccc(C(=O)NC2C/C(=C\c3ccc(F)c(F)c3)C(=O)/C(=C/c3ccc(F)c(F)c3)C2)cc1 ; CC(C)N(CCOc1ccc(C(=O)NC2C/C(=C\c3ccc(F)c(F)c3)C(=O)/C(=C/c3ccc(F)c(F)c3)C2)cc1)C(C)C ; CCN(CCOc1ccc(C(=O)NC2C/C(=C\c3ccc(F)c(F)c3)C(=O)/C(=C/c3ccc(F)c(F)c3)C2)cc1)C1CCCCC1] 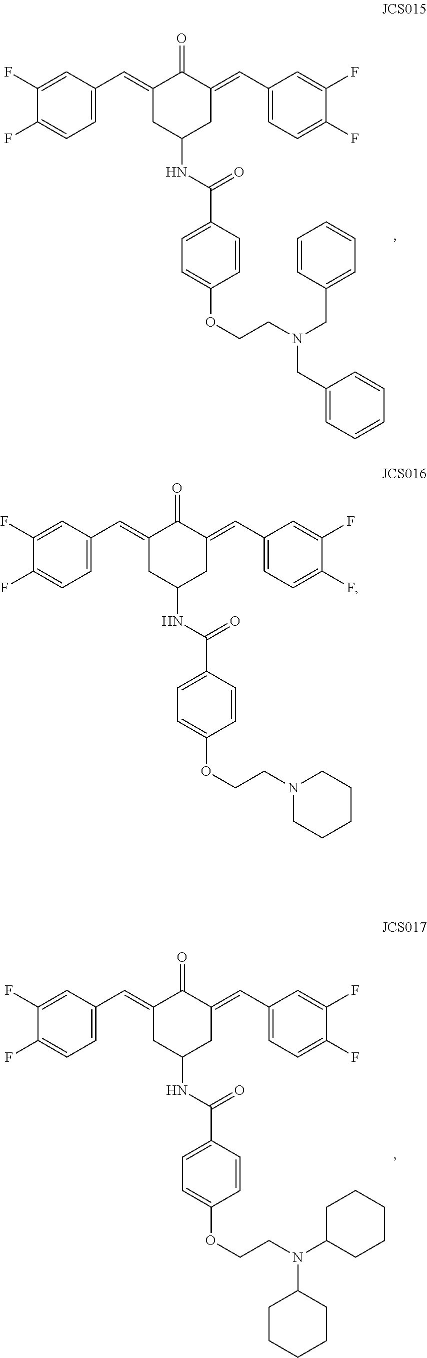 [SMILES: O=C1/C(=C/c2ccc(F)c(F)c2)CC(NC(=O)c2ccc(OCCN(Cc3ccccc3)Cc3ccccc3)cc2)C/C1=C\c1ccc(F)c(F)c1 ; O=C1/C(=C/c2ccc(F)c(F)c2)CC(NC(=O)c2ccc(OCCN3CCCCC3)cc2)C/C1=C\c1ccc(F)c(F)c1 ; O=C1/C(=C/c2ccc(F)c(F)c2)CC(NC(=O)c2ccc(OCCN(C3CCCCC3)C3CCCCC3)cc2)C/C1=C\c1ccc(F)c(F)c1] 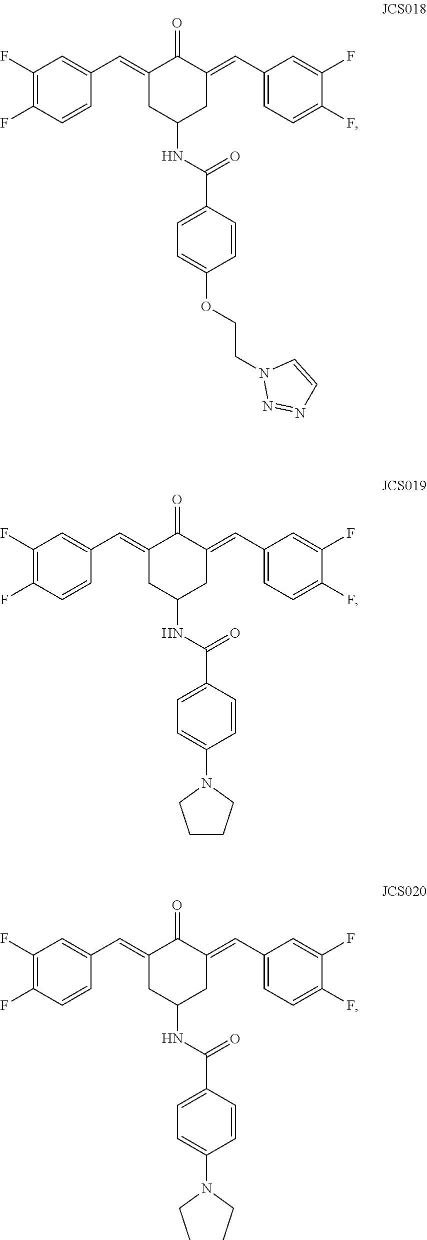 [SMILES: O=C1/C(=C/c2ccc(F)c(F)c2)CC(NC(=O)c2ccc(OCCn3ccnn3)cc2)C/C1=C\c1ccc(F)c(F)c1 ; O=C1/C(=C/c2ccc(F)c(F)c2)CC(NC(=O)c2ccc(N3CCCC3)cc2)C/C1=C\c1ccc(F)c(F)c1 ; CCN(CC)c1ccc(C(=O)NC2C/C(=C\c3ccc(F)c(F)c3)C(=O)/C(=C/c3ccc(F)c(F)c3)C2)cc1] 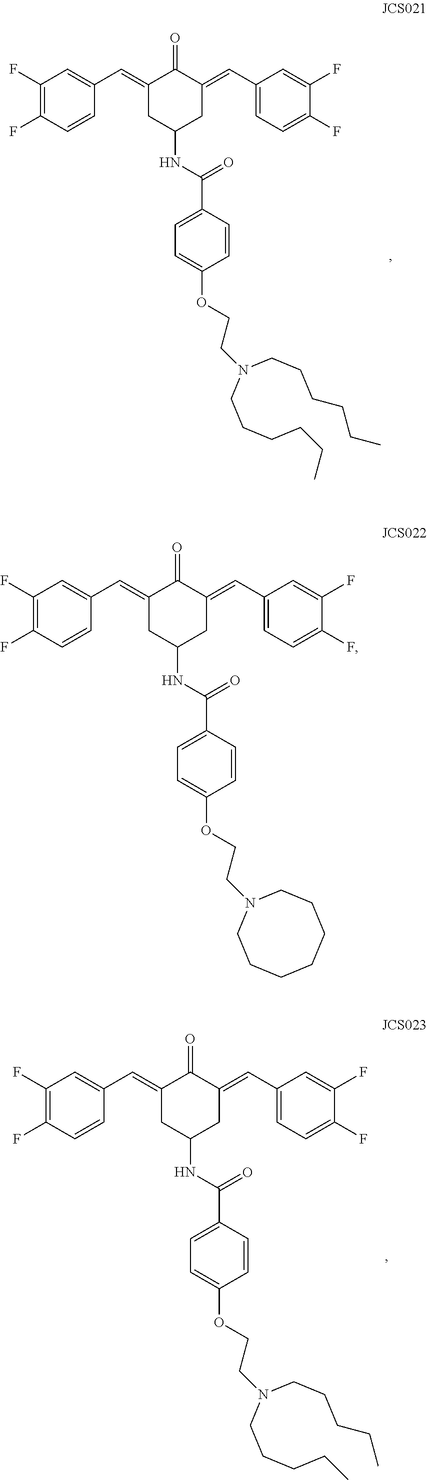 [SMILES: CCCCCCN(CCCCCC)CCOc1ccc(C(=O)NC2C/C(=C\c3ccc(F)c(F)c3)C(=O)/C(=C/c3ccc(F)c(F)c3)C2)cc1 ; O=C1/C(=C/c2ccc(F)c(F)c2)CC(NC(=O)c2ccc(OCCN3CCCCCCC3)cc2)C/C1=C\c1ccc(F)c(F)c1 ; CCCCCN(CCCCC)CCOc1ccc(C(=O)NC2C/C(=C\c3ccc(F)c(F)c3)C(=O)/C(=C/c3ccc(F)c(F)c3)C2)cc1] 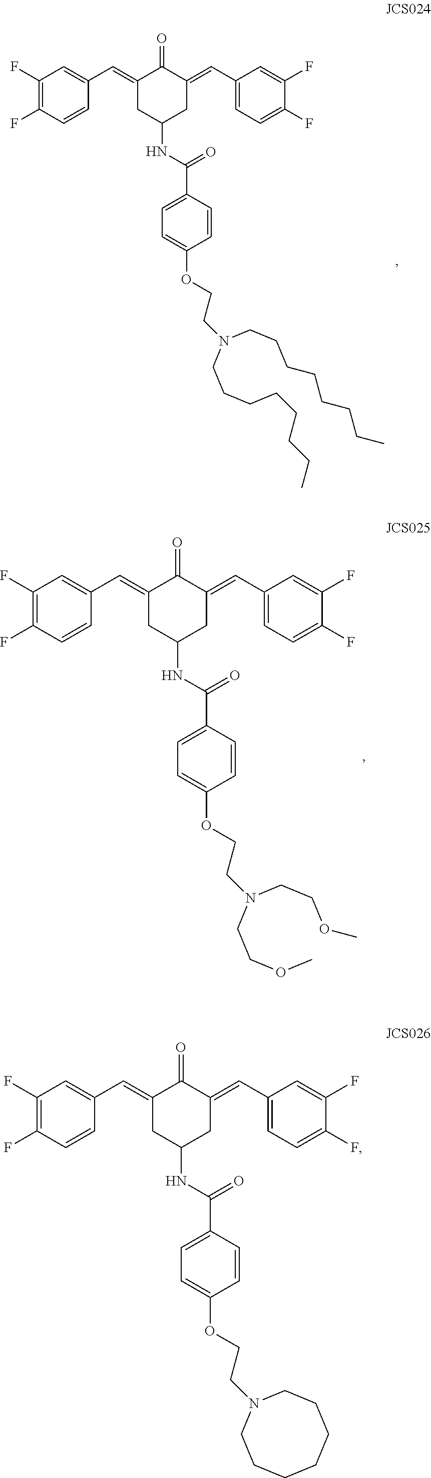 [SMILES: CCCCCCCCN(CCCCCCCC)CCOc1ccc(C(=O)NC2C/C(=C\c3ccc(F)c(F)c3)C(=O)/C(=C/c3ccc(F)c(F)c3)C2)cc1 ; COCCN(CCOC)CCOc1ccc(C(=O)NC2C/C(=C\c3ccc(F)c(F)c3)C(=O)/C(=C/c3ccc(F)c(F)c3)C2)cc1 ; O=C1/C(=C/c2ccc(F)c(F)c2)CC(NC(=O)c2ccc(OCCN3CCCCCCC3)cc2)C/C1=C\c1ccc(F)c(F)c1] 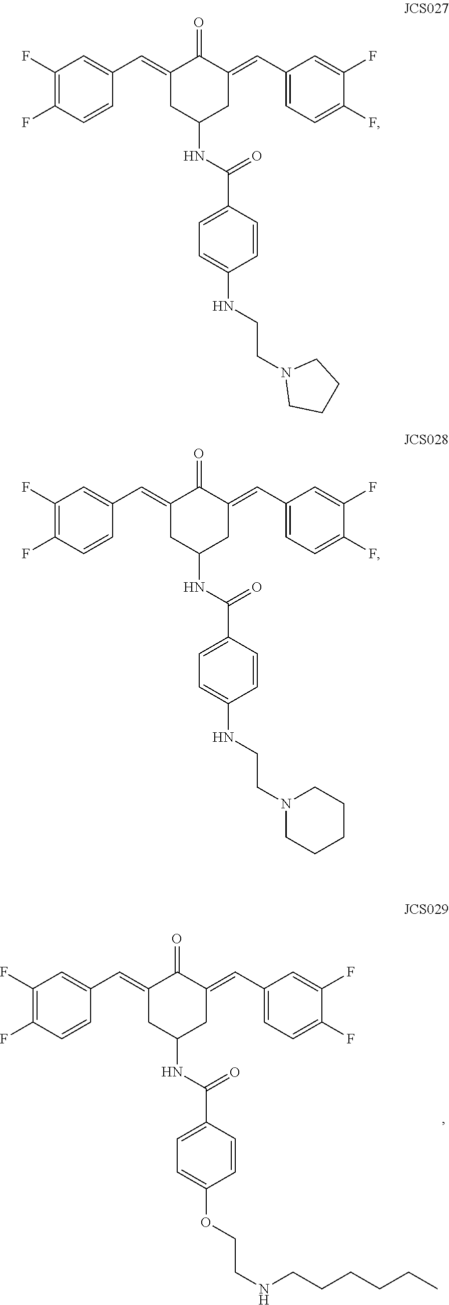 [SMILES: O=C1/C(=C/c2ccc(F)c(F)c2)CC(NC(=O)c2ccc(NCCN3CCCC3)cc2)C/C1=C\c1ccc(F)c(F)c1 ; O=C1/C(=C/c2ccc(F)c(F)c2)CC(NC(=O)c2ccc(NCCN3CCCCC3)cc2)C/C1=C\c1ccc(F)c(F)c1 ; CCCCCCNCCOc1ccc(C(=O)NC2C/C(=C\c3ccc(F)c(F)c3)C(=O)/C(=C/c3ccc(F)c(F)c3)C2)cc1] 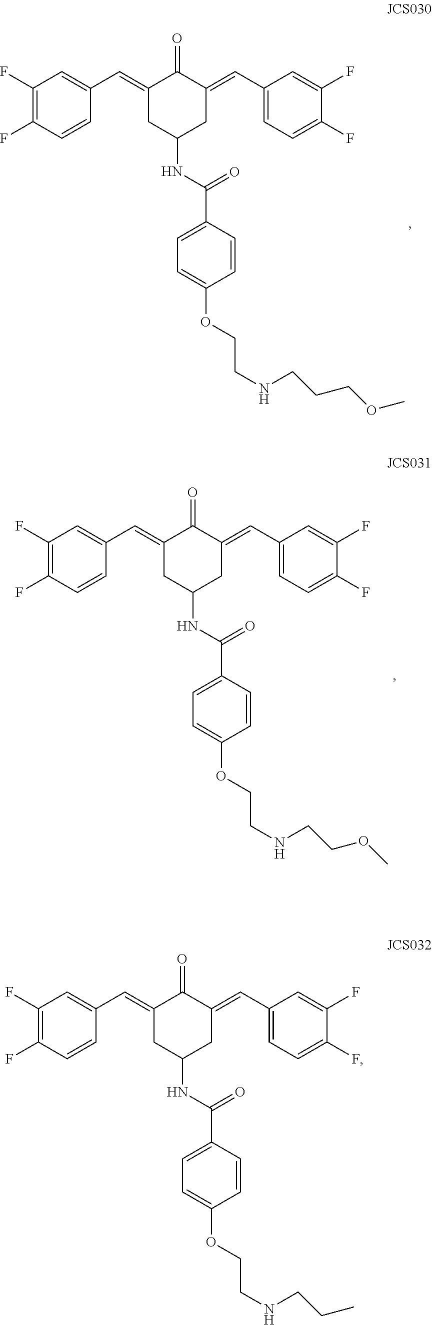 [SMILES: COCCCNCCOc1ccc(C(=O)NC2C/C(=C\c3ccc(F)c(F)c3)C(=O)/C(=C/c3ccc(F)c(F)c3)C2)cc1 ; COCCNCCOc1ccc(C(=O)NC2C/C(=C\c3ccc(F)c(F)c3)C(=O)/C(=C/c3ccc(F)c(F)c3)C2)cc1 ; CCCNCCOc1ccc(C(=O)NC2C/C(=C\c3ccc(F)c(F)c3)C(=O)/C(=C/c3ccc(F)c(F)c3)C2)cc1] 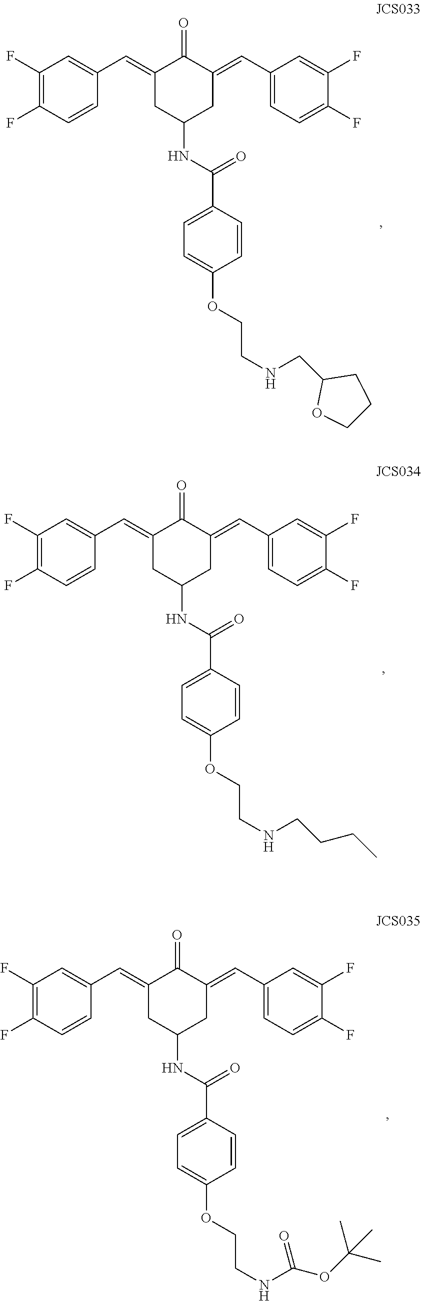 [SMILES: O=C1/C(=C/c2ccc(F)c(F)c2)CC(NC(=O)c2ccc(OCCNCC3CCCO3)cc2)C/C1=C\c1ccc(F)c(F)c1 ; CCCCNCCOc1ccc(C(=O)NC2C/C(=C\c3ccc(F)c(F)c3)C(=O)/C(=C/c3ccc(F)c(F)c3)C2)cc1 ; CC(C)(C)OC(=O)NCCOc1ccc(C(=O)NC2C/C(=C\c3ccc(F)c(F)c3)C(=O)/C(=C/c3ccc(F)c(F)c3)C2)cc1] 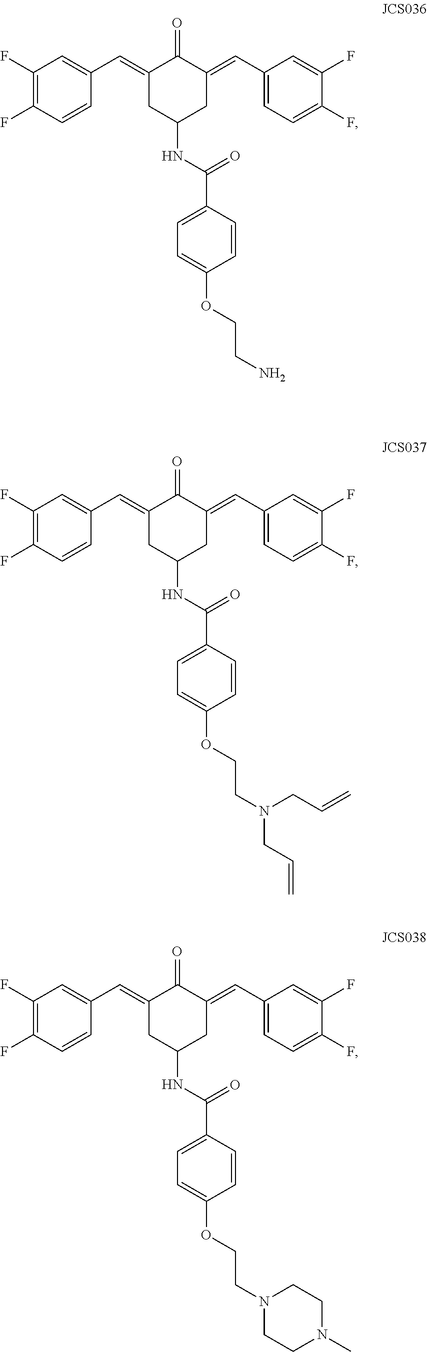 [SMILES: NCCOc1ccc(C(=O)NC2C/C(=C\c3ccc(F)c(F)c3)C(=O)/C(=C/c3ccc(F)c(F)c3)C2)cc1 ; C=CCN(CC=C)CCOc1ccc(C(=O)NC2C/C(=C\c3ccc(F)c(F)c3)C(=O)/C(=C/c3ccc(F)c(F)c3)C2)cc1 ; CN1CCN(CCOc2ccc(C(=O)NC3C/C(=C\c4ccc(F)c(F)c4)C(=O)/C(=C/c4ccc(F)c(F)c4)C3)cc2)CC1] 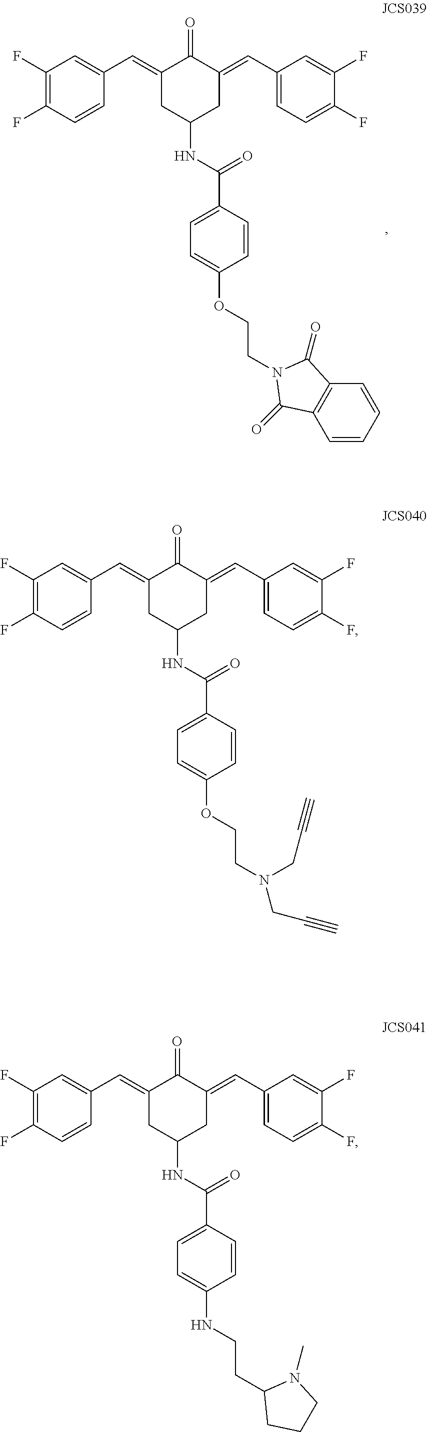 [SMILES: O=C1/C(=C/c2ccc(F)c(F)c2)CC(NC(=O)c2ccc(OCCN3C(=O)c4ccccc4C3=O)cc2)C/C1=C\c1ccc(F)c(F)c1 ; C#CCN(CC#C)CCOc1ccc(C(=O)NC2C/C(=C\c3ccc(F)c(F)c3)C(=O)/C(=C/c3ccc(F)c(F)c3)C2)cc1 ; CN1CCCC1CCNc1ccc(C(=O)NC2C/C(=C\c3ccc(F)c(F)c3)C(=O)/C(=C/c3ccc(F)c(F)c3)C2)cc1] 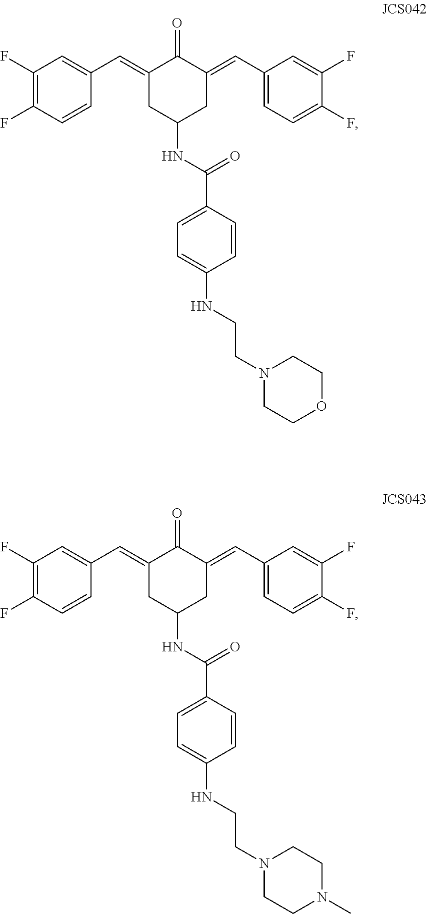 [SMILES: O=C1/C(=C/c2ccc(F)c(F)c2)CC(NC(=O)c2ccc(NCCN3CCOCC3)cc2)C/C1=C\c1ccc(F)c(F)c1 ; CN1CCN(CCNc2ccc(C(=O)NC3C/C(=C\c4ccc(F)c(F)c4)C(=O)/C(=C/c4ccc(F)c(F)c4)C3)cc2)CC1] 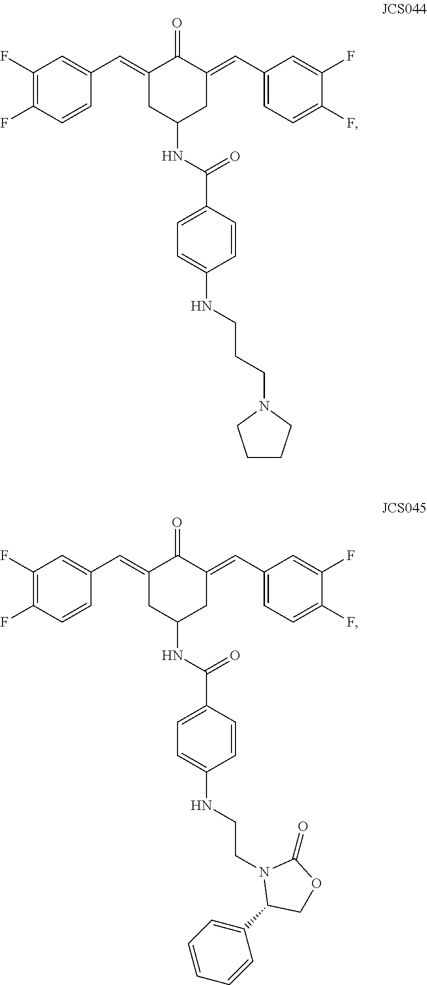 [SMILES: O=C1/C(=C/c2ccc(F)c(F)c2)CC(NC(=O)c2ccc(NCCCN3CCCC3)cc2)C/C1=C\c1ccc(F)c(F)c1 ; O=C1/C(=C/c2ccc(F)c(F)c2)CC(NC(=O)c2ccc(NCCN3C(=O)OC[C@@H]3c3ccccc3)cc2)C/C1=C\c1ccc(F)c(F)c1] 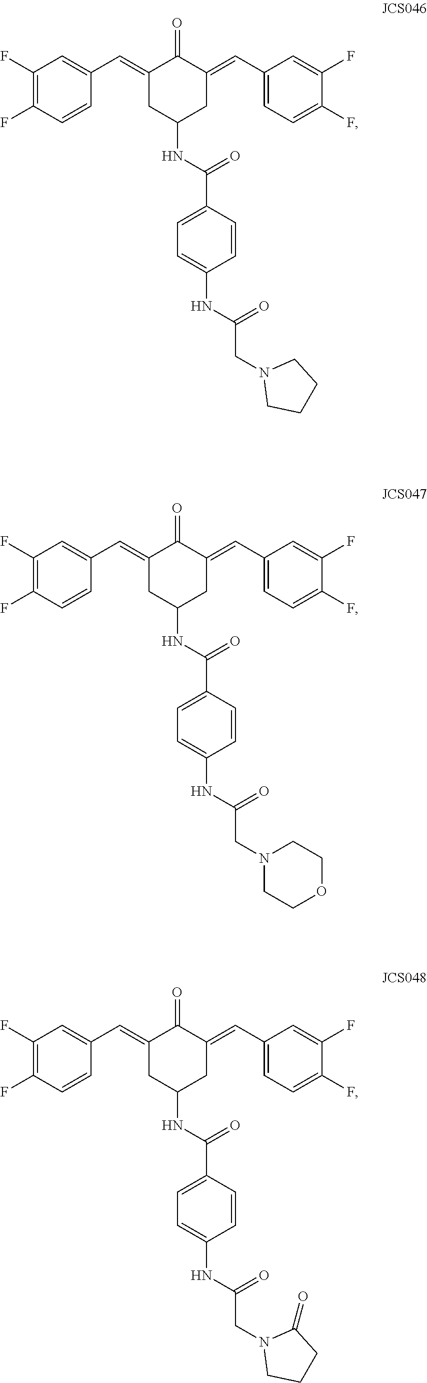 [SMILES: O=C(CN1CCCC1)Nc1ccc(C(=O)NC2C/C(=C\c3ccc(F)c(F)c3)C(=O)/C(=C/c3ccc(F)c(F)c3)C2)cc1 ; O=C(CN1CCOCC1)Nc1ccc(C(=O)NC2C/C(=C\c3ccc(F)c(F)c3)C(=O)/C(=C/c3ccc(F)c(F)c3)C2)cc1 ; O=C(CN1CCCC1=O)Nc1ccc(C(=O)NC2C/C(=C\c3ccc(F)c(F)c3)C(=O)/C(=C/c3ccc(F)c(F)c3)C2)cc1] 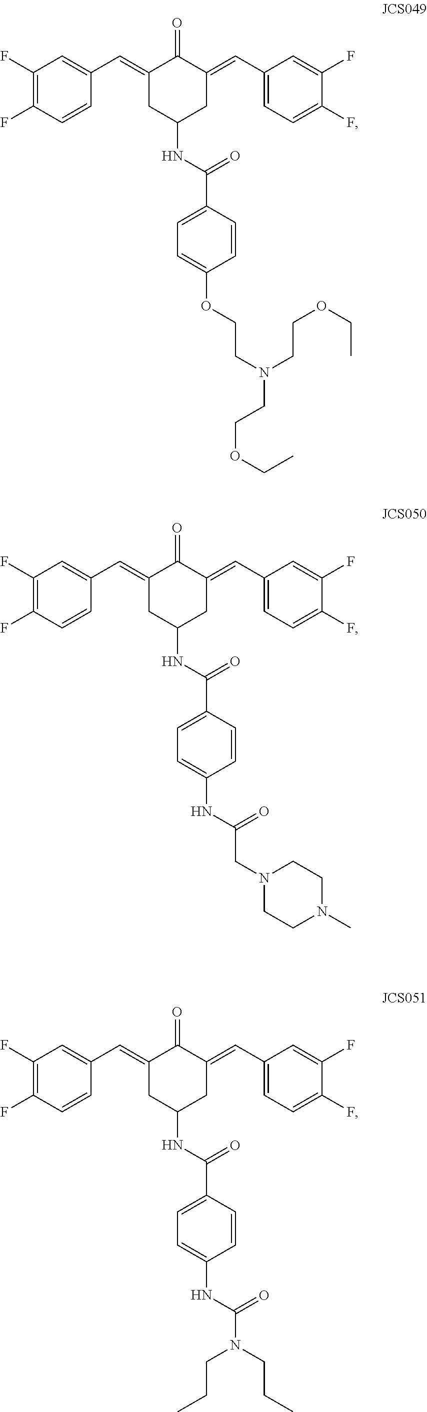 [SMILES: CCOCCN(CCOCC)CCOc1ccc(C(=O)NC2C/C(=C\c3ccc(F)c(F)c3)C(=O)/C(=C/c3ccc(F)c(F)c3)C2)cc1 ; CN1CCN(CC(=O)Nc2ccc(C(=O)NC3C/C(=C\c4ccc(F)c(F)c4)C(=O)/C(=C/c4ccc(F)c(F)c4)C3)cc2)CC1 ; CCCN(CCC)C(=O)Nc1ccc(C(=O)NC2C/C(=C\c3ccc(F)c(F)c3)C(=O)/C(=C/c3ccc(F)c(F)c3)C2)cc1] 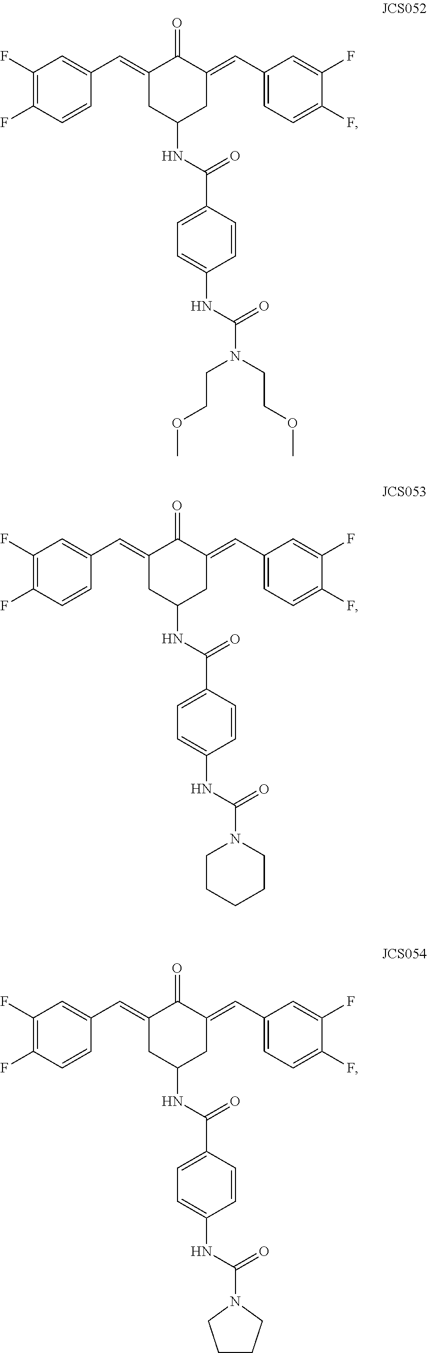 [SMILES: COCCN(CCOC)C(=O)Nc1ccc(C(=O)NC2C/C(=C\c3ccc(F)c(F)c3)C(=O)/C(=C/c3ccc(F)c(F)c3)C2)cc1 ; O=C1/C(=C/c2ccc(F)c(F)c2)CC(NC(=O)c2ccc(NC(=O)N3CCCCC3)cc2)C/C1=C\c1ccc(F)c(F)c1 ; O=C1/C(=C/c2ccc(F)c(F)c2)CC(NC(=O)c2ccc(NC(=O)N3CCCC3)cc2)C/C1=C\c1ccc(F)c(F)c1] 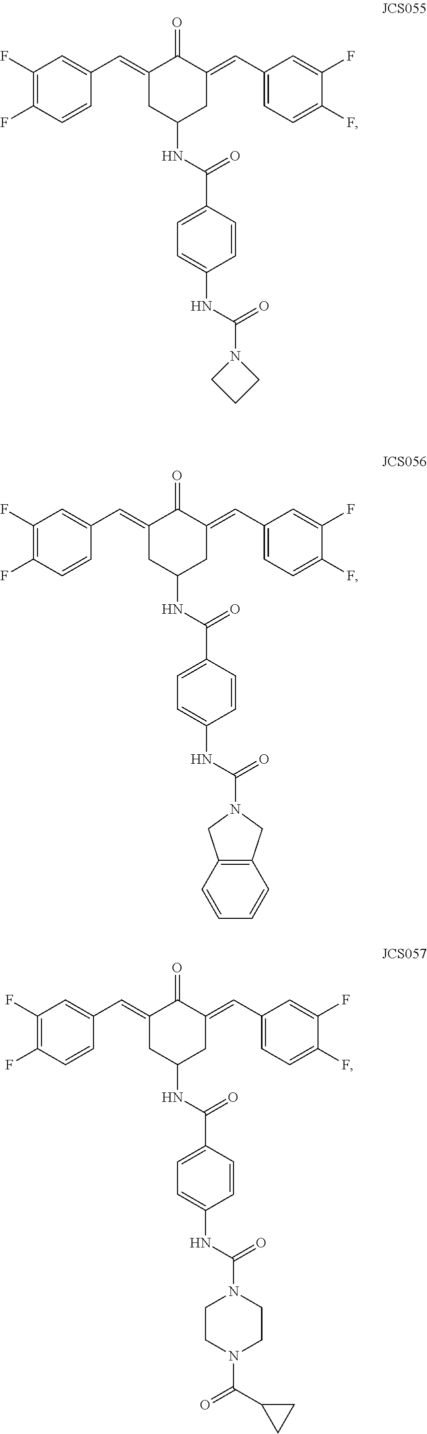 [SMILES: O=C1/C(=C/c2ccc(F)c(F)c2)CC(NC(=O)c2ccc(NC(=O)N3CCC3)cc2)C/C1=C\c1ccc(F)c(F)c1 ; O=C1/C(=C/c2ccc(F)c(F)c2)CC(NC(=O)c2ccc(NC(=O)N3Cc4ccccc4C3)cc2)C/C1=C\c1ccc(F)c(F)c1 ; O=C1/C(=C/c2ccc(F)c(F)c2)CC(NC(=O)c2ccc(NC(=O)N3CCN(C(=O)C4CC4)CC3)cc2)C/C1=C\c1ccc(F)c(F)c1] 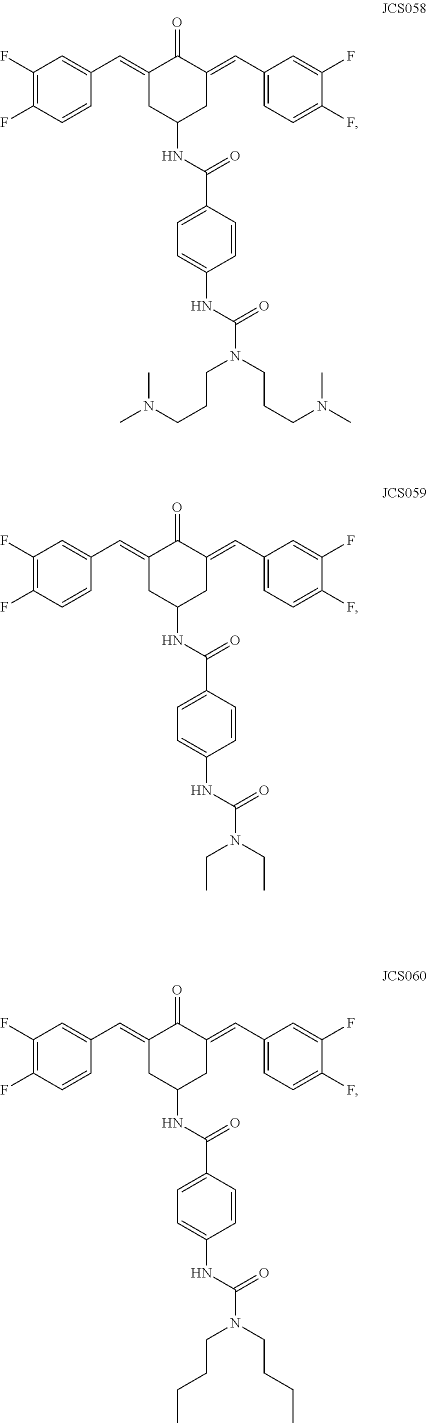 [SMILES: CN(C)CCCN(CCCN(C)C)C(=O)Nc1ccc(C(=O)NC2C/C(=C\c3ccc(F)c(F)c3)C(=O)/C(=C/c3ccc(F)c(F)c3)C2)cc1 ; CCN(CC)C(=O)Nc1ccc(C(=O)NC2C/C(=C\c3ccc(F)c(F)c3)C(=O)/C(=C/c3ccc(F)c(F)c3)C2)cc1 ; CCCCN(CCCC)C(=O)Nc1ccc(C(=O)NC2C/C(=C\c3ccc(F)c(F)c3)C(=O)/C(=C/c3ccc(F)c(F)c3)C2)cc1] 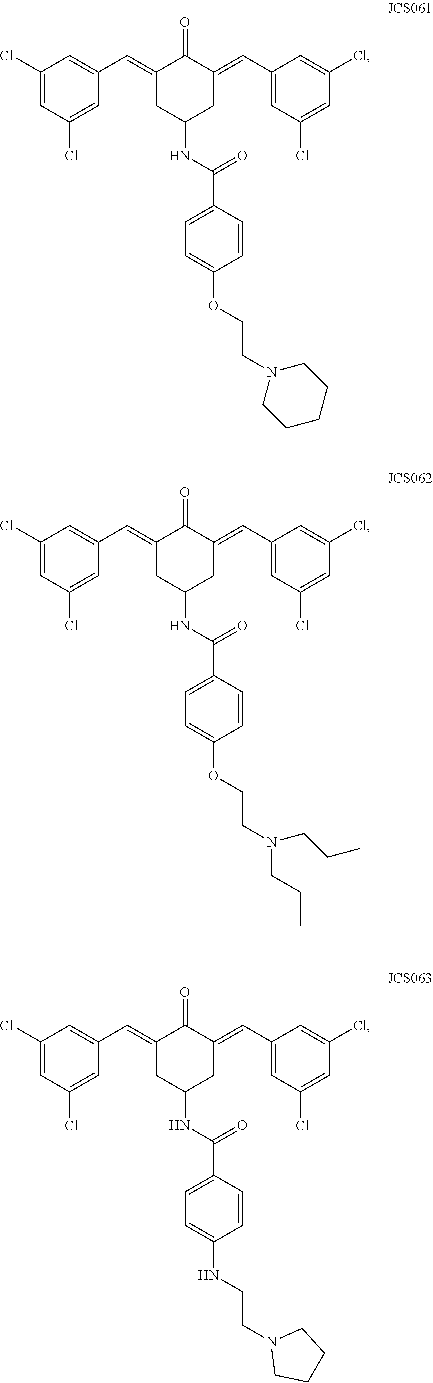 [SMILES: O=C1/C(=C/c2cc(Cl)cc(Cl)c2)CC(NC(=O)c2ccc(OCCN3CCCCC3)cc2)C/C1=C\c1cc(Cl)cc(Cl)c1 ; CCCN(CCC)CCOc1ccc(C(=O)NC2C/C(=C\c3cc(Cl)cc(Cl)c3)C(=O)/C(=C/c3cc(Cl)cc(Cl)c3)C2)cc1 ; O=C1/C(=C/c2cc(Cl)cc(Cl)c2)CC(NC(=O)c2ccc(NCCN3CCCC3)cc2)C/C1=C\c1cc(Cl)cc(Cl)c1] 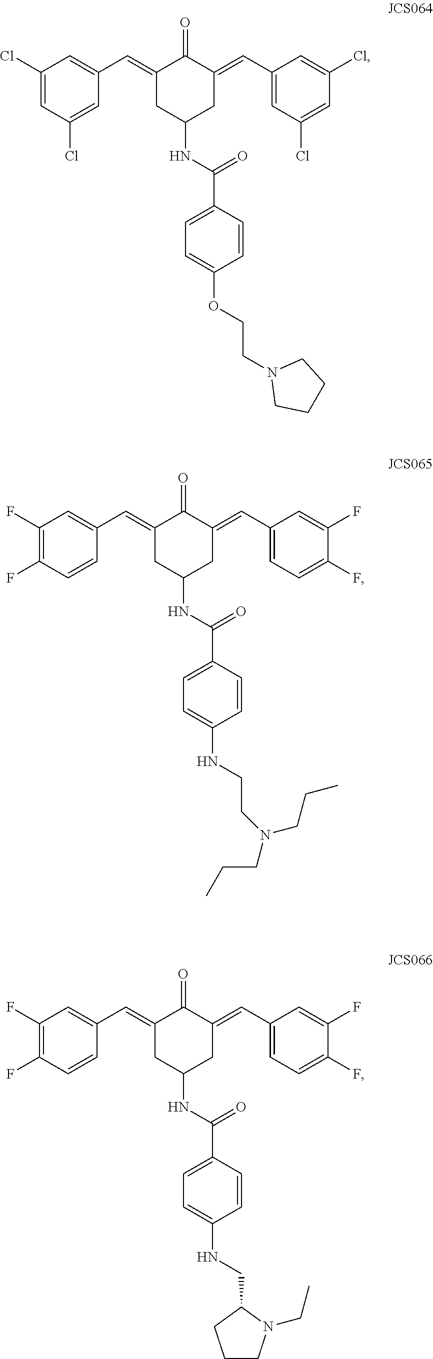 [SMILES: O=C1/C(=C/c2cc(Cl)cc(Cl)c2)CC(NC(=O)c2ccc(OCCN3CCCC3)cc2)C/C1=C\c1cc(Cl)cc(Cl)c1 ; CCCN(CCC)CCNc1ccc(C(=O)NC2C/C(=C\c3ccc(F)c(F)c3)C(=O)/C(=C/c3ccc(F)c(F)c3)C2)cc1 ; CCN1CCC[C@@H]1CNc1ccc(C(=O)NC2C/C(=C\c3ccc(F)c(F)c3)C(=O)/C(=C/c3ccc(F)c(F)c3)C2)cc1] 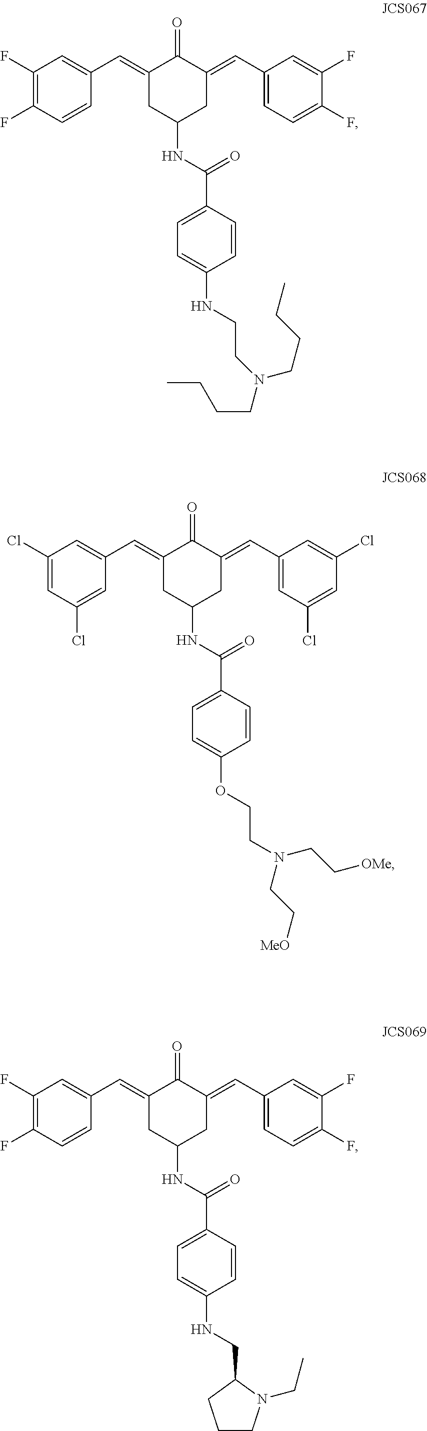 [SMILES: CCCCN(CCCC)CCNc1ccc(C(=O)NC2C/C(=C\c3ccc(F)c(F)c3)C(=O)/C(=C/c3ccc(F)c(F)c3)C2)cc1 ; COCCN(CCOC)CCOc1ccc(C(=O)NC2C/C(=C\c3cc(Cl)cc(Cl)c3)C(=O)/C(=C/c3cc(Cl)cc(Cl)c3)C2)cc1 ; CCN1CCC[C@H]1CNc1ccc(C(=O)NC2C/C(=C\c3ccc(F)c(F)c3)C(=O)/C(=C/c3ccc(F)c(F)c3)C2)cc1] 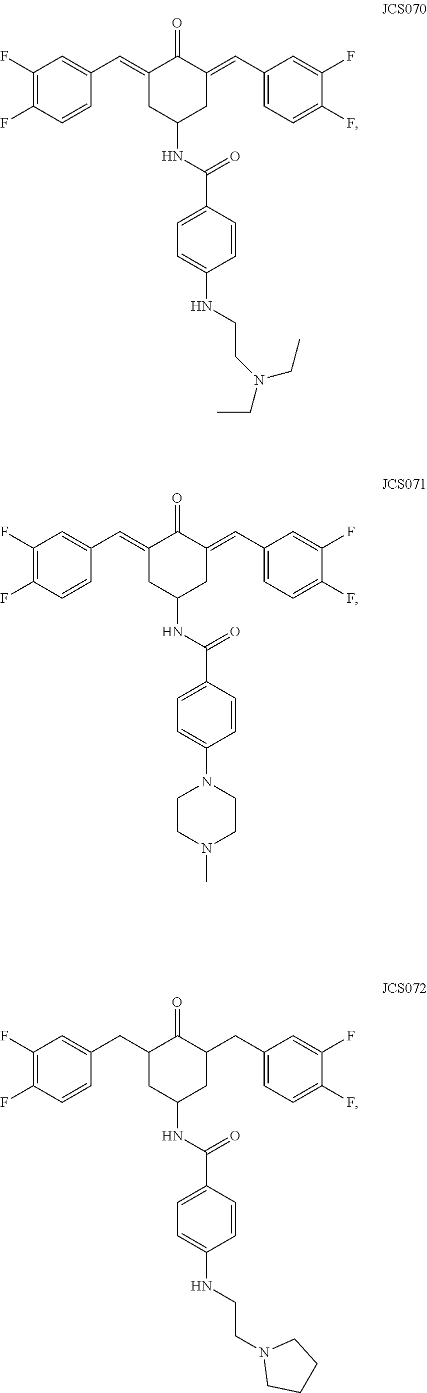 [SMILES: CCN(CC)CCNc1ccc(C(=O)NC2C/C(=C\c3ccc(F)c(F)c3)C(=O)/C(=C/c3ccc(F)c(F)c3)C2)cc1 ; CN1CCN(c2ccc(C(=O)NC3C/C(=C\c4ccc(F)c(F)c4)C(=O)/C(=C/c4ccc(F)c(F)c4)C3)cc2)CC1 ; O=C(NC1CC(Cc2ccc(F)c(F)c2)C(=O)C(Cc2ccc(F)c(F)c2)C1)c1ccc(NCCN2CCCC2)cc1] 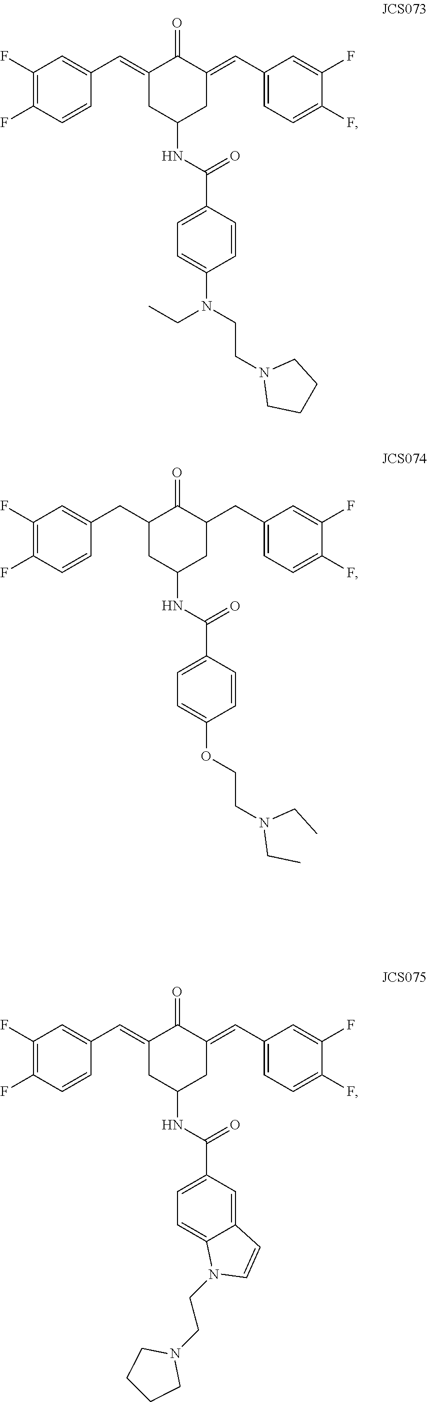 [SMILES: CCN(CCN1CCCC1)c1ccc(C(=O)NC2C/C(=C\c3ccc(F)c(F)c3)C(=O)/C(=C/c3ccc(F)c(F)c3)C2)cc1 ; CCN(CC)CCOc1ccc(C(=O)NC2CC(Cc3ccc(F)c(F)c3)C(=O)C(Cc3ccc(F)c(F)c3)C2)cc1 ; O=C1/C(=C/c2ccc(F)c(F)c2)CC(NC(=O)c2ccc3c(ccn3CCN3CCCC3)c2)C/C1=C\c1ccc(F)c(F)c1] 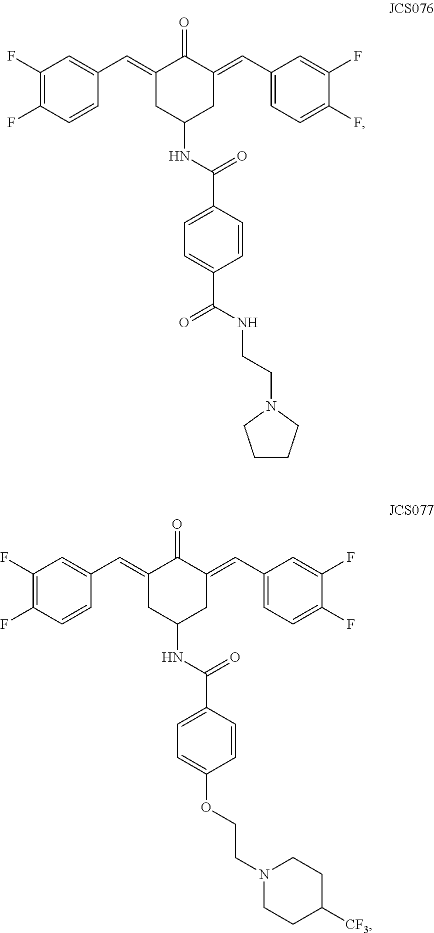 [SMILES: O=C1/C(=C/c2ccc(F)c(F)c2)CC(NC(=O)c2ccc(C(=O)NCCN3CCCC3)cc2)C/C1=C\c1ccc(F)c(F)c1 ; O=C1/C(=C/c2ccc(F)c(F)c2)CC(NC(=O)c2ccc(OCCN3CCC(C(F)(F)F)CC3)cc2)C/C1=C\c1ccc(F)c(F)c1] 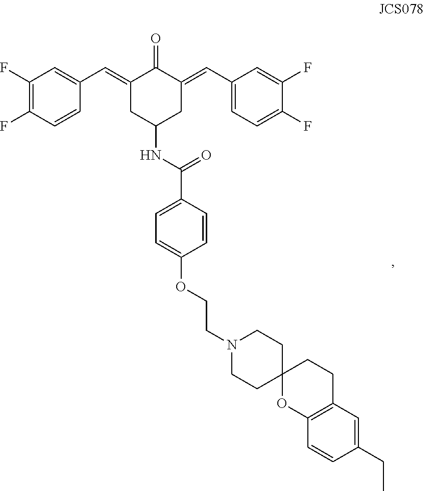 [SMILES: CCc1ccc2c(c1)CCC1(CCN(CCOc3ccc(C(=O)NC4C/C(=C\c5ccc(F)c(F)c5)C(=O)/C(=C/c5ccc(F)c(F)c5)C4)cc3)CC1)O2] 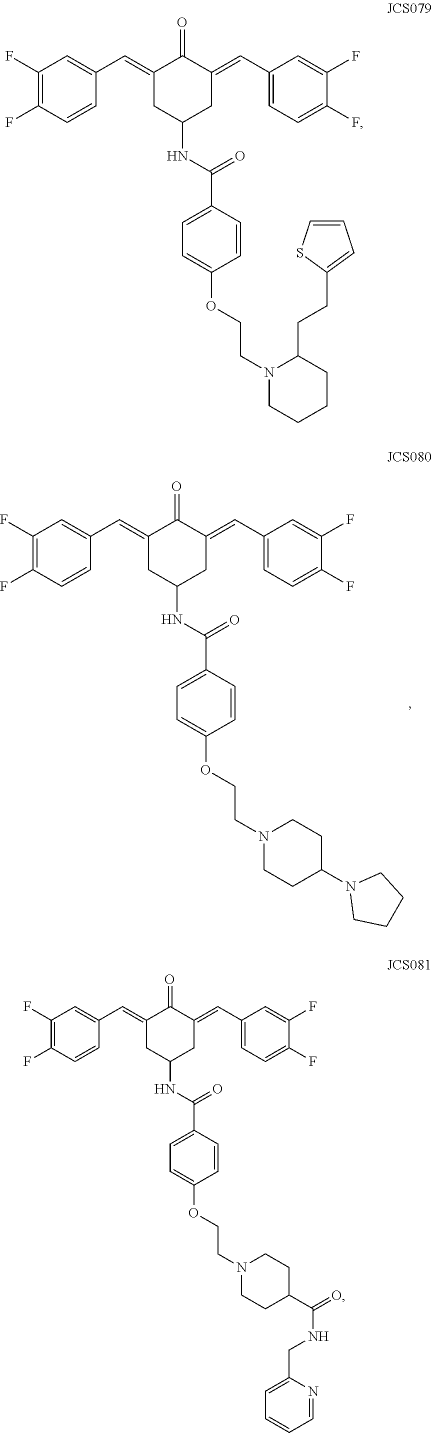 [SMILES: O=C1/C(=C/c2ccc(F)c(F)c2)CC(NC(=O)c2ccc(OCCN3CCCCC3CCc3cccs3)cc2)C/C1=C\c1ccc(F)c(F)c1 ; O=C1/C(=C/c2ccc(F)c(F)c2)CC(NC(=O)c2ccc(OCCN3CCC(N4CCCC4)CC3)cc2)C/C1=C\c1ccc(F)c(F)c1 ; O=C1/C(=C/c2ccc(F)c(F)c2)CC(NC(=O)c2ccc(OCCN3CCC(C(=O)NCc4ccccn4)CC3)cc2)C/C1=C\c1ccc(F)c(F)c1] 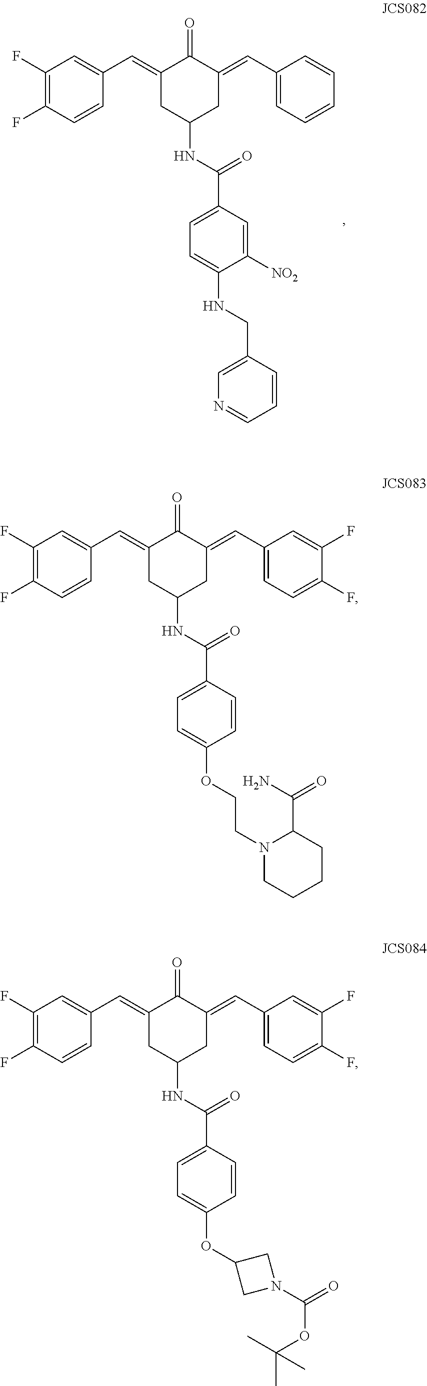 [SMILES: O=C1/C(=C/c2ccccc2)CC(NC(=O)c2ccc(NCc3cccnc3)c([N+](=O)[O-])c2)C/C1=C\c1ccc(F)c(F)c1 ; NC(=O)C1CCCCN1CCOc1ccc(C(=O)NC2C/C(=C\c3ccc(F)c(F)c3)C(=O)/C(=C/c3ccc(F)c(F)c3)C2)cc1 ; CC(C)(C)OC(=O)N1CC(Oc2ccc(C(=O)NC3C/C(=C\c4ccc(F)c(F)c4)C(=O)/C(=C/c4ccc(F)c(F)c4)C3)cc2)C1] 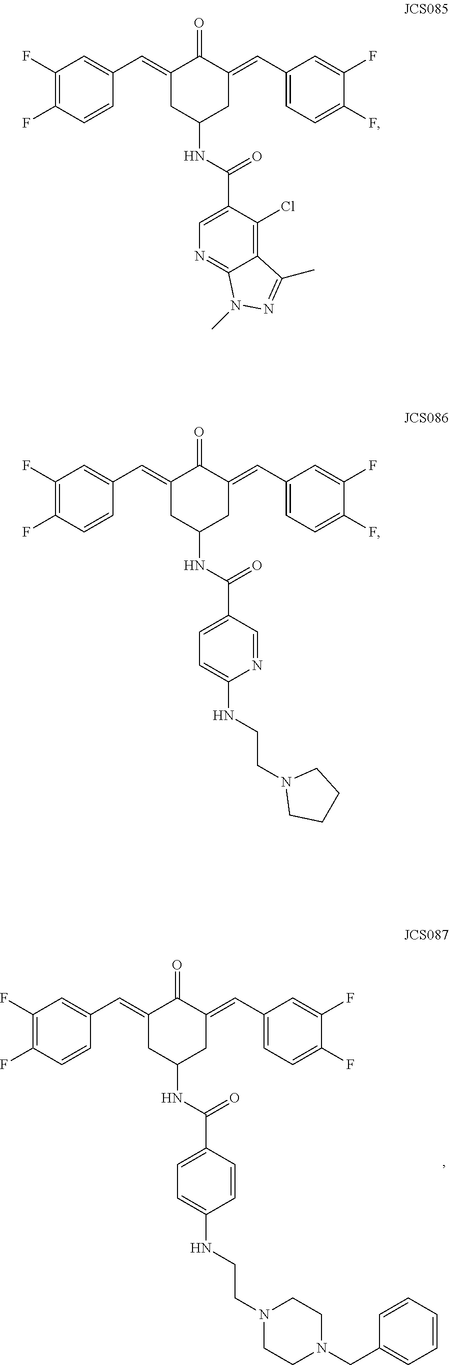 [SMILES: Cc1nn(C)c2ncc(C(=O)NC3C/C(=C\c4ccc(F)c(F)c4)C(=O)/C(=C/c4ccc(F)c(F)c4)C3)c(Cl)c12 ; O=C1/C(=C/c2ccc(F)c(F)c2)CC(NC(=O)c2ccc(NCCN3CCCC3)nc2)C/C1=C\c1ccc(F)c(F)c1 ; O=C1/C(=C/c2ccc(F)c(F)c2)CC(NC(=O)c2ccc(NCCN3CCN(Cc4ccccc4)CC3)cc2)C/C1=C\c1ccc(F)c(F)c1] 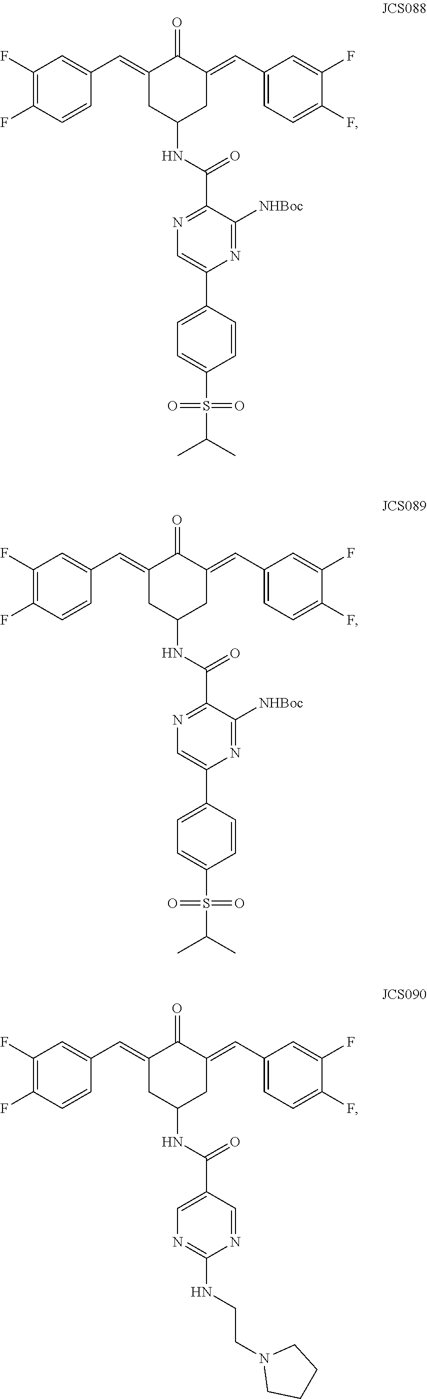 [SMILES: CC(C)S(=O)(=O)c1ccc(-c2cnc(C(=O)NC3C/C(=C\c4ccc(F)c(F)c4)C(=O)/C(=C/c4ccc(F)c(F)c4)C3)c(NC(=O)OC(C)(C)C)n2)cc1 ; CC(C)S(=O)(=O)c1ccc(-c2cnc(C(=O)NC3C/C(=C\c4ccc(F)c(F)c4)C(=O)/C(=C/c4ccc(F)c(F)c4)C3)c(NC(=O)OC(C)(C)C)n2)cc1 ; O=C1/C(=C/c2ccc(F)c(F)c2)CC(NC(=O)c2cnc(NCCN3CCCC3)nc2)C/C1=C\c1ccc(F)c(F)c1] 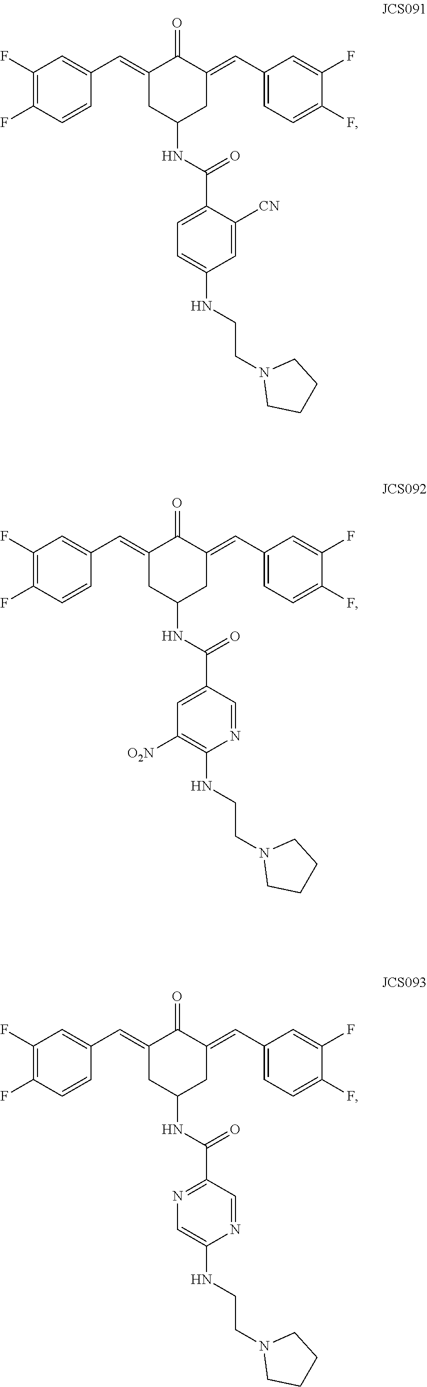 [SMILES: N#Cc1cc(NCCN2CCCC2)ccc1C(=O)NC1C/C(=C\c2ccc(F)c(F)c2)C(=O)/C(=C/c2ccc(F)c(F)c2)C1 ; O=C1/C(=C/c2ccc(F)c(F)c2)CC(NC(=O)c2cnc(NCCN3CCCC3)c([N+](=O)[O-])c2)C/C1=C\c1ccc(F)c(F)c1 ; O=C1/C(=C/c2ccc(F)c(F)c2)CC(NC(=O)c2cnc(NCCN3CCCC3)cn2)C/C1=C\c1ccc(F)c(F)c1] 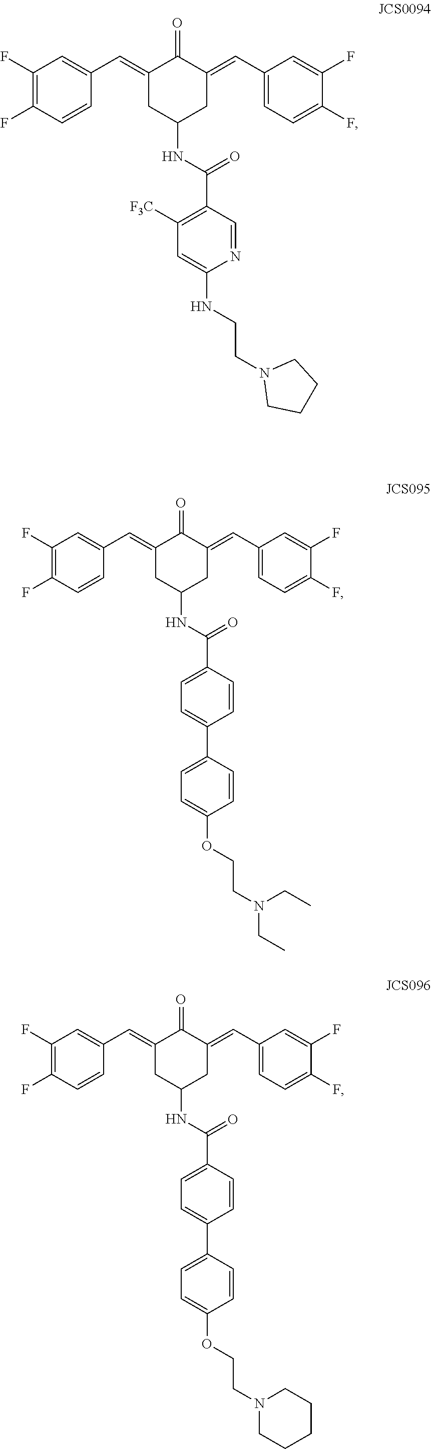 [SMILES: O=C1/C(=C/c2ccc(F)c(F)c2)CC(NC(=O)c2cnc(NCCN3CCCC3)cc2C(F)(F)F)C/C1=C\c1ccc(F)c(F)c1 ; CCN(CC)CCOc1ccc(-c2ccc(C(=O)NC3C/C(=C\c4ccc(F)c(F)c4)C(=O)/C(=C/c4ccc(F)c(F)c4)C3)cc2)cc1 ; O=C1/C(=C/c2ccc(F)c(F)c2)CC(NC(=O)c2ccc(-c3ccc(OCCN4CCCCC4)cc3)cc2)C/C1=C\c1ccc(F)c(F)c1] 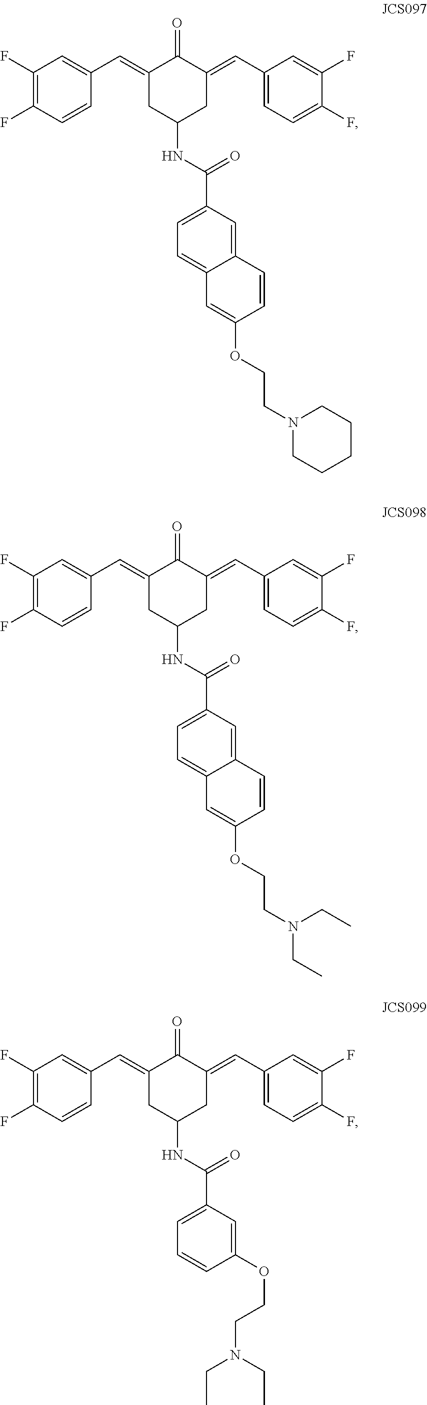 [SMILES: O=C1/C(=C/c2ccc(F)c(F)c2)CC(NC(=O)c2ccc3cc(OCCN4CCCCC4)ccc3c2)C/C1=C\c1ccc(F)c(F)c1 ; CCN(CC)CCOc1ccc2cc(C(=O)NC3C/C(=C\c4ccc(F)c(F)c4)C(=O)/C(=C/c4ccc(F)c(F)c4)C3)ccc2c1 ; CCN(CC)CCOc1cccc(C(=O)NC2C/C(=C\c3ccc(F)c(F)c3)C(=O)/C(=C/c3ccc(F)c(F)c3)C2)c1] 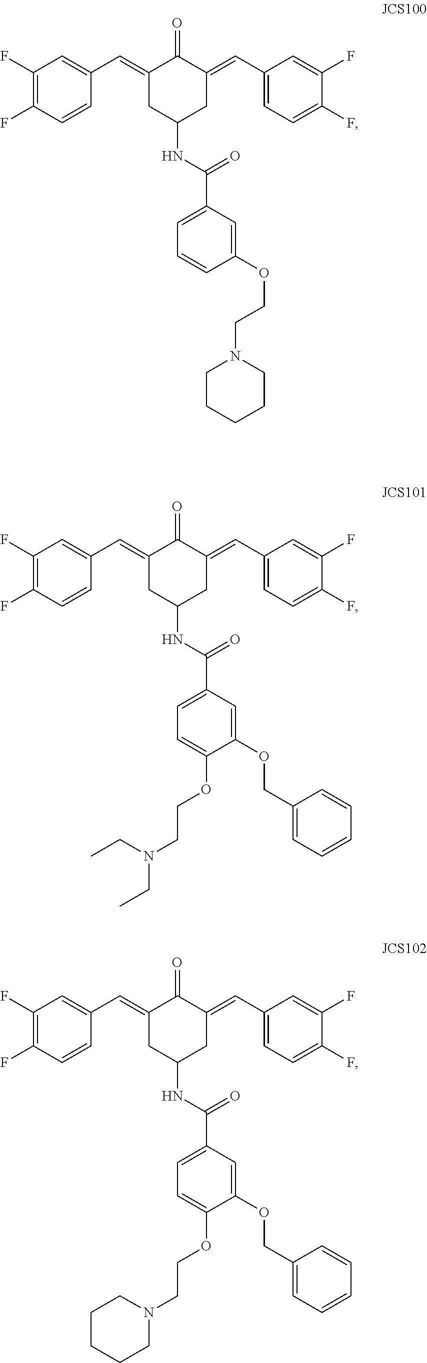 [SMILES: O=C1/C(=C/c2ccc(F)c(F)c2)CC(NC(=O)c2cccc(OCCN3CCCCC3)c2)C/C1=C\c1ccc(F)c(F)c1 ; CCN(CC)CCOc1ccc(C(=O)NC2C/C(=C\c3ccc(F)c(F)c3)C(=O)/C(=C/c3ccc(F)c(F)c3)C2)cc1OCc1ccccc1 ; O=C1/C(=C/c2ccc(F)c(F)c2)CC(NC(=O)c2ccc(OCCN3CCCCC3)c(OCc3ccccc3)c2)C/C1=C\c1ccc(F)c(F)c1] 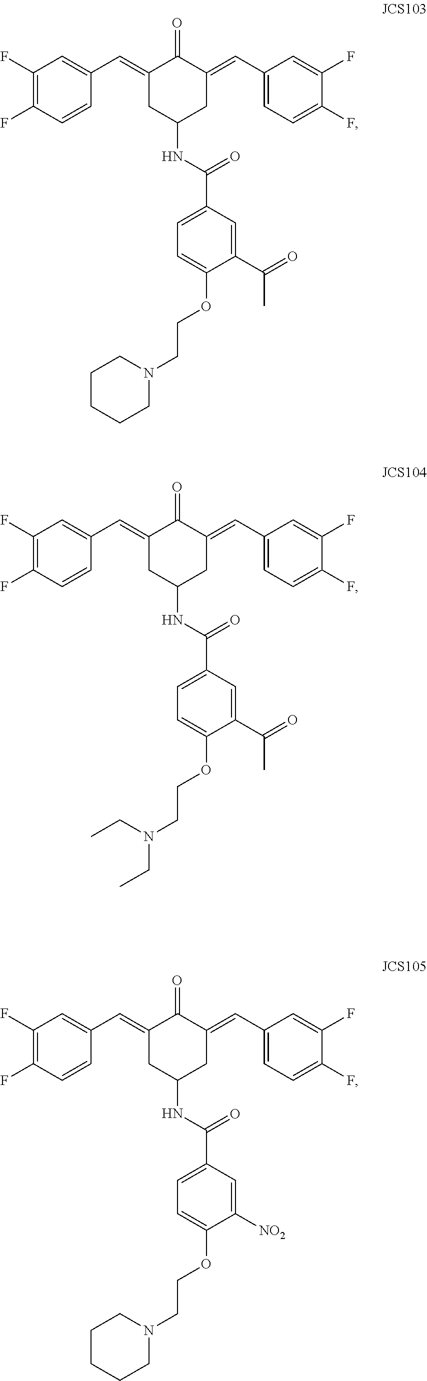 [SMILES: CC(=O)c1cc(C(=O)NC2C/C(=C\c3ccc(F)c(F)c3)C(=O)/C(=C/c3ccc(F)c(F)c3)C2)ccc1OCCN1CCCCC1 ; CCN(CC)CCOc1ccc(C(=O)NC2C/C(=C\c3ccc(F)c(F)c3)C(=O)/C(=C/c3ccc(F)c(F)c3)C2)cc1C(C)=O ; O=C1/C(=C/c2ccc(F)c(F)c2)CC(NC(=O)c2ccc(OCCN3CCCCC3)c([N+](=O)[O-])c2)C/C1=C\c1ccc(F)c(F)c1] 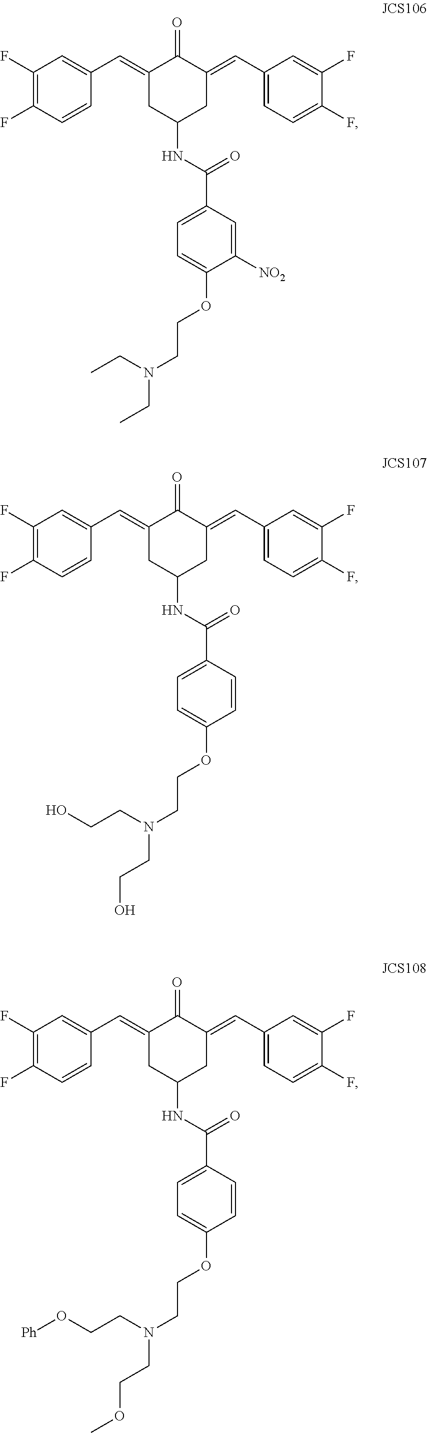 [SMILES: CCN(CC)CCOc1ccc(C(=O)NC2C/C(=C\c3ccc(F)c(F)c3)C(=O)/C(=C/c3ccc(F)c(F)c3)C2)cc1[N+](=O)[O-] ; O=C1/C(=C/c2ccc(F)c(F)c2)CC(NC(=O)c2ccc(OCCN(CCO)CCO)cc2)C/C1=C\c1ccc(F)c(F)c1 ; COCCN(CCOc1ccccc1)CCOc1ccc(C(=O)NC2C/C(=C\c3ccc(F)c(F)c3)C(=O)/C(=C/c3ccc(F)c(F)c3)C2)cc1] 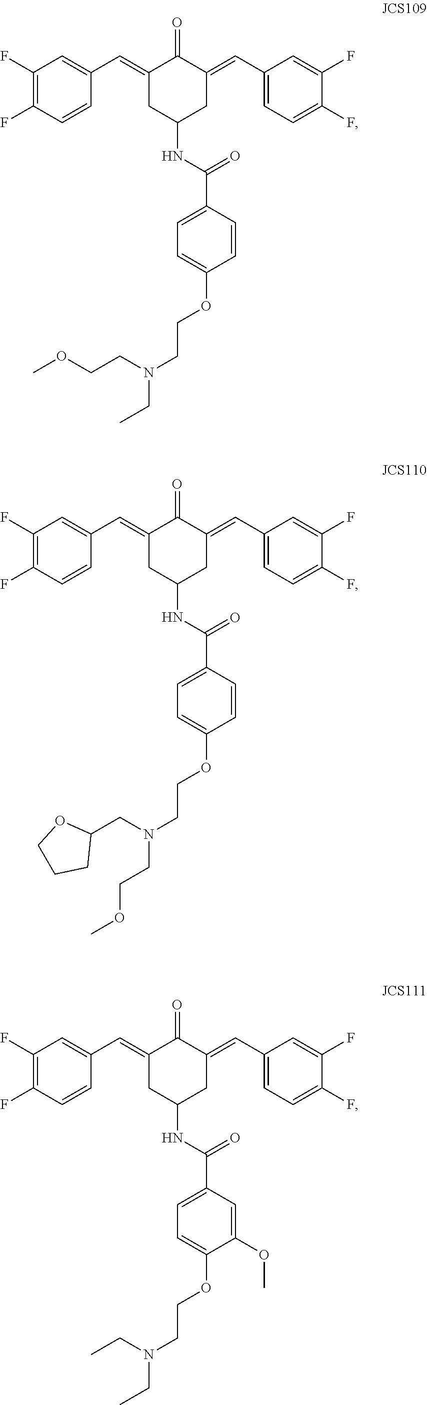 [SMILES: CCN(CCOC)CCOc1ccc(C(=O)NC2C/C(=C\c3ccc(F)c(F)c3)C(=O)/C(=C/c3ccc(F)c(F)c3)C2)cc1 ; COCCN(CCOc1ccc(C(=O)NC2C/C(=C\c3ccc(F)c(F)c3)C(=O)/C(=C/c3ccc(F)c(F)c3)C2)cc1)CC1CCCO1 ; CCN(CC)CCOc1ccc(C(=O)NC2C/C(=C\c3ccc(F)c(F)c3)C(=O)/C(=C/c3ccc(F)c(F)c3)C2)cc1OC] 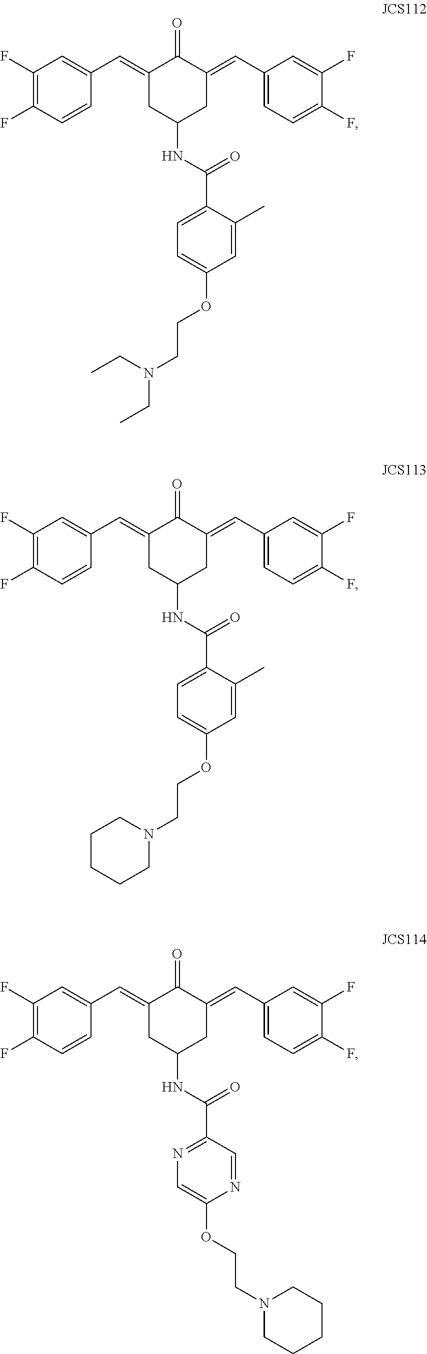 [SMILES: CCN(CC)CCOc1ccc(C(=O)NC2C/C(=C\c3ccc(F)c(F)c3)C(=O)/C(=C/c3ccc(F)c(F)c3)C2)c(C)c1 ; Cc1cc(OCCN2CCCCC2)ccc1C(=O)NC1C/C(=C\c2ccc(F)c(F)c2)C(=O)/C(=C/c2ccc(F)c(F)c2)C1 ; O=C1/C(=C/c2ccc(F)c(F)c2)CC(NC(=O)c2cnc(OCCN3CCCCC3)cn2)C/C1=C\c1ccc(F)c(F)c1] 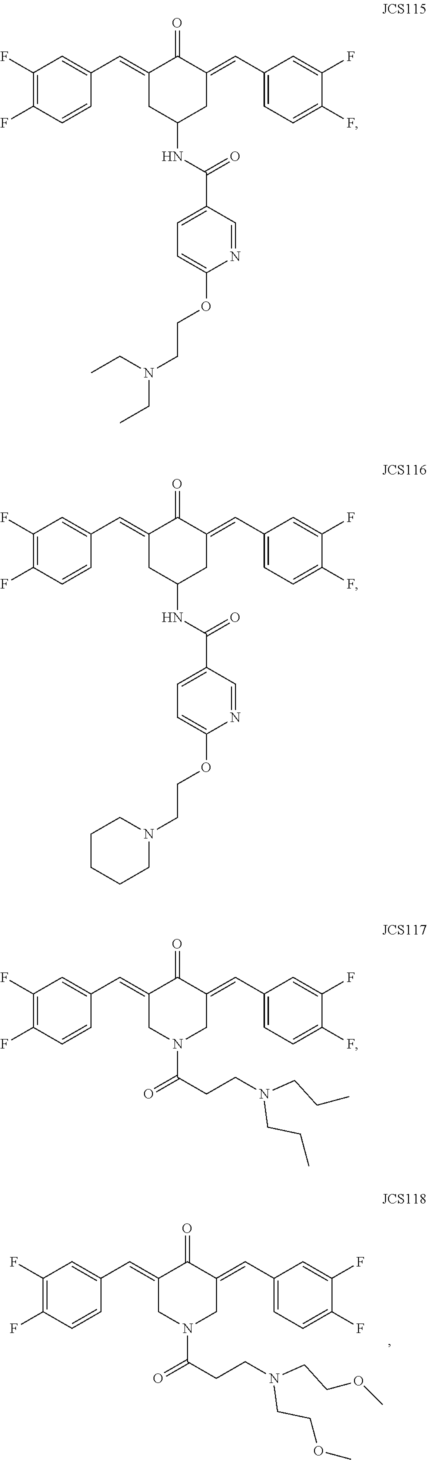 [SMILES: CCN(CC)CCOc1ccc(C(=O)NC2C/C(=C\c3ccc(F)c(F)c3)C(=O)/C(=C/c3ccc(F)c(F)c3)C2)cn1 ; O=C1/C(=C/c2ccc(F)c(F)c2)CC(NC(=O)c2ccc(OCCN3CCCCC3)nc2)C/C1=C\c1ccc(F)c(F)c1 ; CCCN(CCC)CCC(=O)N1C/C(=C\c2ccc(F)c(F)c2)C(=O)/C(=C/c2ccc(F)c(F)c2)C1 ; COCCN(CCOC)CCC(=O)N1C/C(=C\c2ccc(F)c(F)c2)C(=O)/C(=C/c2ccc(F)c(F)c2)C1] 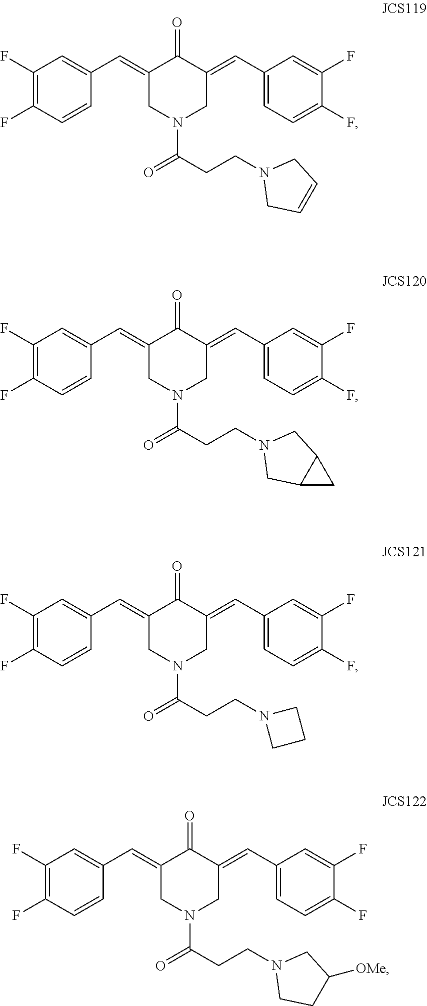 [SMILES: O=C1/C(=C/c2ccc(F)c(F)c2)CN(C(=O)CCN2CC=CC2)C/C1=C\c1ccc(F)c(F)c1 ; O=C1/C(=C/c2ccc(F)c(F)c2)CN(C(=O)CCN2CC3CC3C2)C/C1=C\c1ccc(F)c(F)c1 ; O=C1/C(=C/c2ccc(F)c(F)c2)CN(C(=O)CCN2CCC2)C/C1=C\c1ccc(F)c(F)c1 ; COC1CCN(CCC(=O)N2C/C(=C\c3ccc(F)c(F)c3)C(=O)/C(=C/c3ccc(F)c(F)c3)C2)C1] 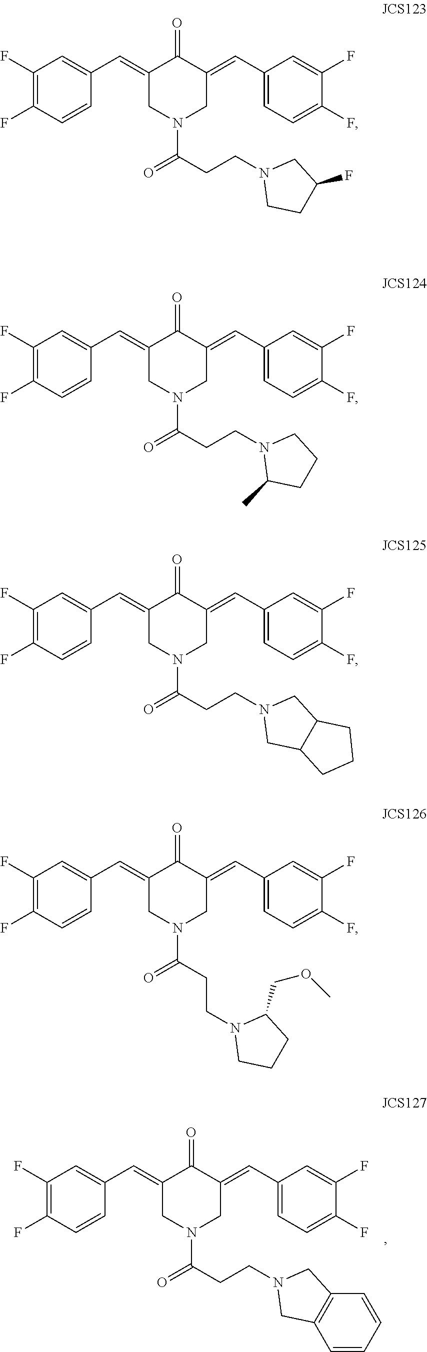 [SMILES: O=C1/C(=C/c2ccc(F)c(F)c2)CN(C(=O)CCN2CC[C@H](F)C2)C/C1=C\c1ccc(F)c(F)c1 ; C[C@@H]1CCCN1CCC(=O)N1C/C(=C\c2ccc(F)c(F)c2)C(=O)/C(=C/c2ccc(F)c(F)c2)C1 ; O=C1/C(=C/c2ccc(F)c(F)c2)CN(C(=O)CCN2CC3CCCC3C2)C/C1=C\c1ccc(F)c(F)c1 ; COC[C@@H]1CCCN1CCC(=O)N1C/C(=C\c2ccc(F)c(F)c2)C(=O)/C(=C/c2ccc(F)c(F)c2)C1 ; O=C1/C(=C/c2ccc(F)c(F)c2)CN(C(=O)CCN2Cc3ccccc3C2)C/C1=C\c1ccc(F)c(F)c1] 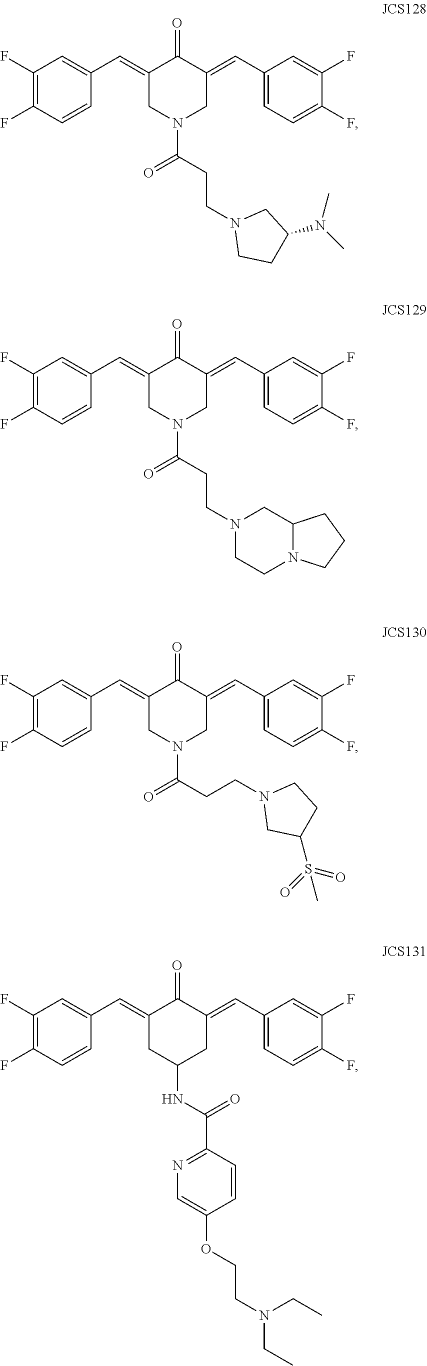 [SMILES: CN(C)[C@@H]1CCN(CCC(=O)N2C/C(=C\c3ccc(F)c(F)c3)C(=O)/C(=C/c3ccc(F)c(F)c3)C2)C1 ; O=C1/C(=C/c2ccc(F)c(F)c2)CN(C(=O)CCN2CCN3CCCC3C2)C/C1=C\c1ccc(F)c(F)c1 ; CS(=O)(=O)C1CCN(CCC(=O)N2C/C(=C\c3ccc(F)c(F)c3)C(=O)/C(=C/c3ccc(F)c(F)c3)C2)C1 ; CCN(CC)CCOc1ccc(C(=O)NC2C/C(=C\c3ccc(F)c(F)c3)C(=O)/C(=C/c3ccc(F)c(F)c3)C2)nc1] 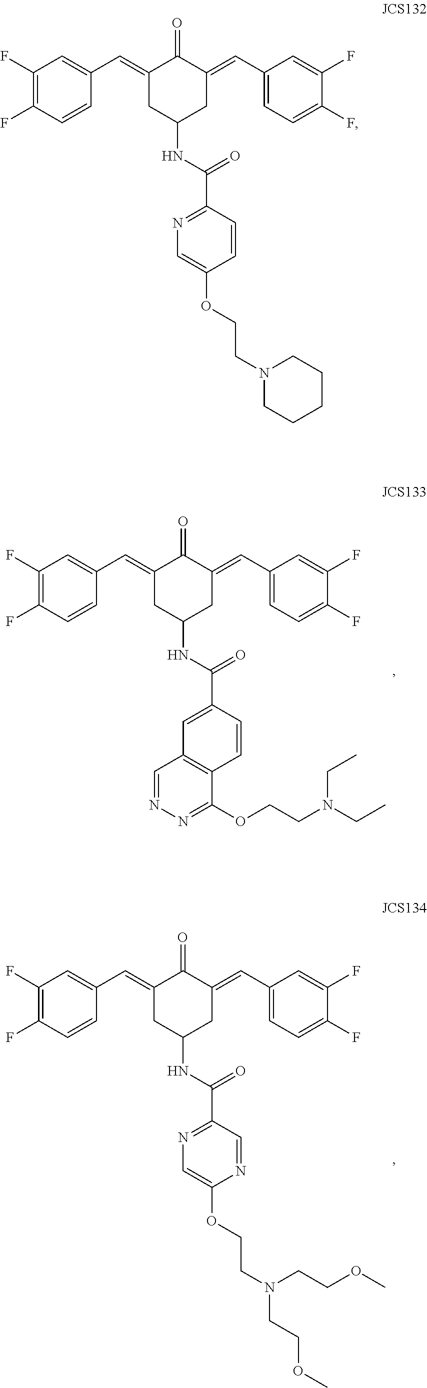 [SMILES: O=C1/C(=C/c2ccc(F)c(F)c2)CC(NC(=O)c2ccc(OCCN3CCCCC3)cn2)C/C1=C\c1ccc(F)c(F)c1 ; CCN(CC)CCOc1nncc2cc(C(=O)NC3C/C(=C\c4ccc(F)c(F)c4)C(=O)/C(=C/c4ccc(F)c(F)c4)C3)ccc12 ; COCCN(CCOC)CCOc1cnc(C(=O)NC2C/C(=C\c3ccc(F)c(F)c3)C(=O)/C(=C/c3ccc(F)c(F)c3)C2)cn1] 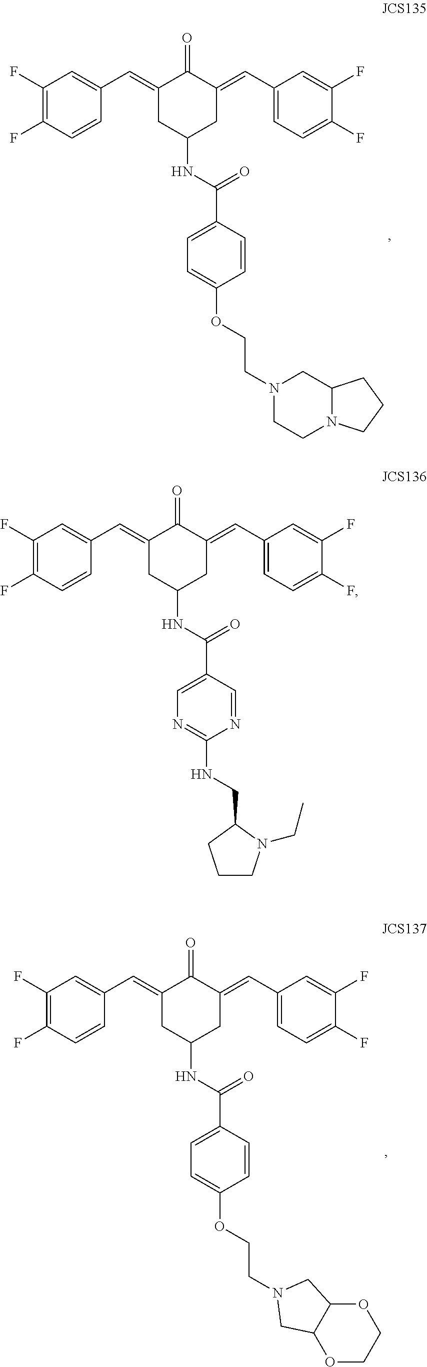 [SMILES: O=C1/C(=C/c2ccc(F)c(F)c2)CC(NC(=O)c2ccc(OCCN3CCN4CCCC4C3)cc2)C/C1=C\c1ccc(F)c(F)c1 ; CCN1CCC[C@H]1CNc1ncc(C(=O)NC2C/C(=C\c3ccc(F)c(F)c3)C(=O)/C(=C/c3ccc(F)c(F)c3)C2)cn1 ; O=C1/C(=C/c2ccc(F)c(F)c2)CC(NC(=O)c2ccc(OCCN3CC4OCCOC4C3)cc2)C/C1=C\c1ccc(F)c(F)c1] 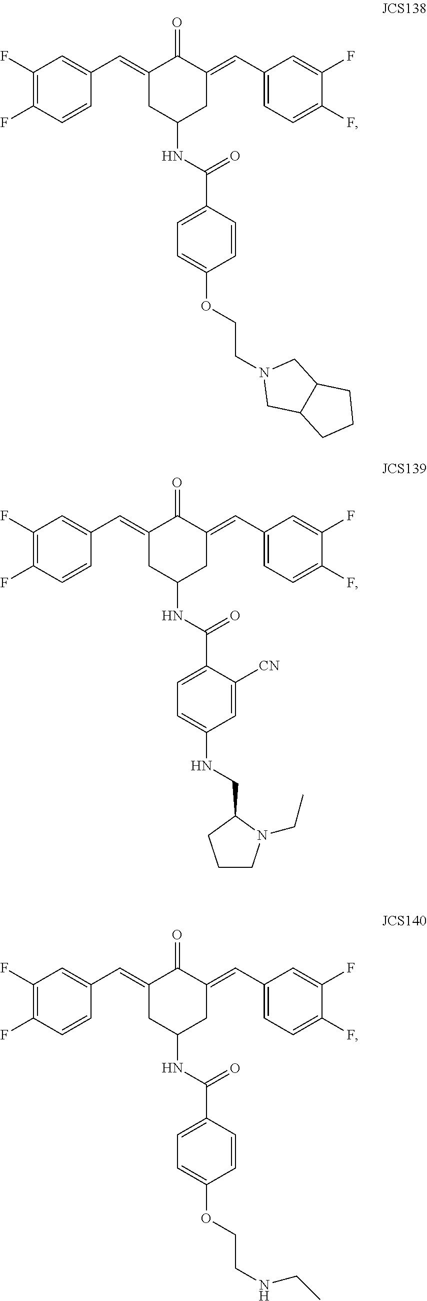 [SMILES: O=C1/C(=C/c2ccc(F)c(F)c2)CC(NC(=O)c2ccc(OCCN3CC4CCCC4C3)cc2)C/C1=C\c1ccc(F)c(F)c1 ; CCN1CCC[C@H]1CNc1ccc(C(=O)NC2C/C(=C\c3ccc(F)c(F)c3)C(=O)/C(=C/c3ccc(F)c(F)c3)C2)c(C#N)c1 ; CCNCCOc1ccc(C(=O)NC2C/C(=C\c3ccc(F)c(F)c3)C(=O)/C(=C/c3ccc(F)c(F)c3)C2)cc1] 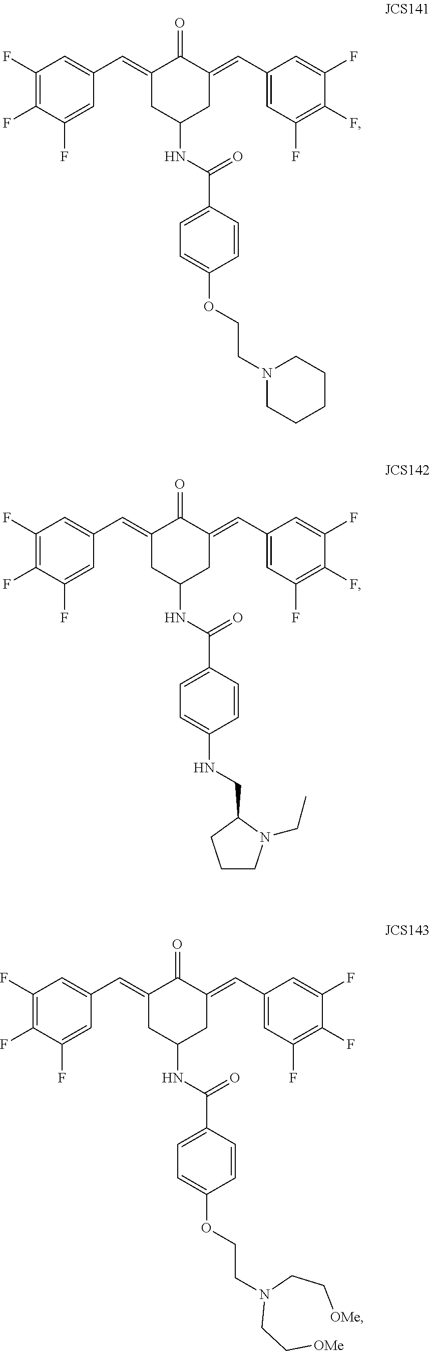 [SMILES: O=C1/C(=C/c2cc(F)c(F)c(F)c2)CC(NC(=O)c2ccc(OCCN3CCCCC3)cc2)C/C1=C\c1cc(F)c(F)c(F)c1 ; CCN1CCC[C@H]1CNc1ccc(C(=O)NC2C/C(=C\c3cc(F)c(F)c(F)c3)C(=O)/C(=C/c3cc(F)c(F)c(F)c3)C2)cc1 ; COCCN(CCOC)CCOc1ccc(C(=O)NC2C/C(=C\c3cc(F)c(F)c(F)c3)C(=O)/C(=C/c3cc(F)c(F)c(F)c3)C2)cc1] 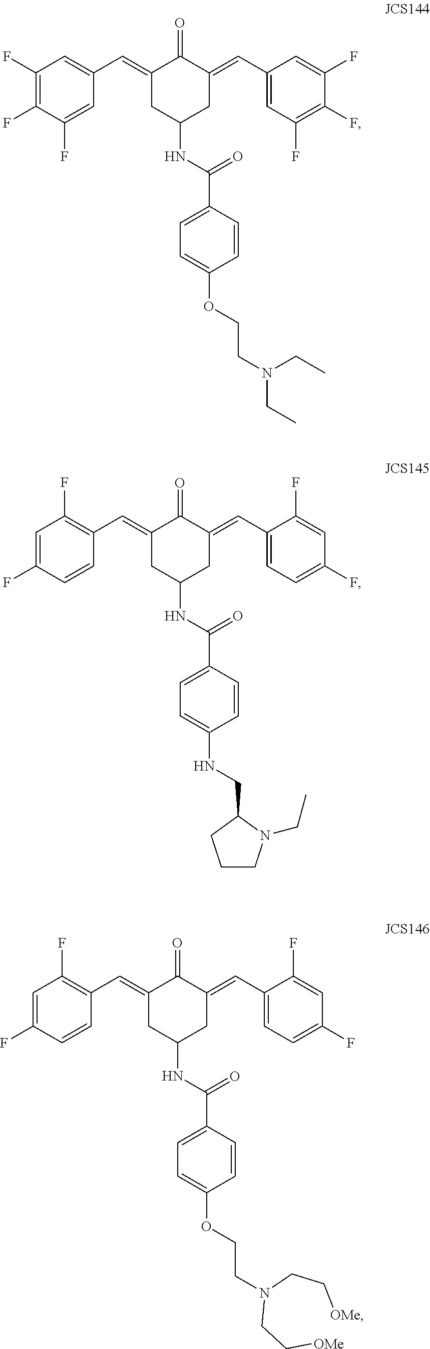 [SMILES: CCN(CC)CCOc1ccc(C(=O)NC2C/C(=C\c3cc(F)c(F)c(F)c3)C(=O)/C(=C/c3cc(F)c(F)c(F)c3)C2)cc1 ; CCN1CCC[C@H]1CNc1ccc(C(=O)NC2C/C(=C\c3ccc(F)cc3F)C(=O)/C(=C/c3ccc(F)cc3F)C2)cc1 ; COCCN(CCOC)CCOc1ccc(C(=O)NC2C/C(=C\c3ccc(F)cc3F)C(=O)/C(=C/c3ccc(F)cc3F)C2)cc1] 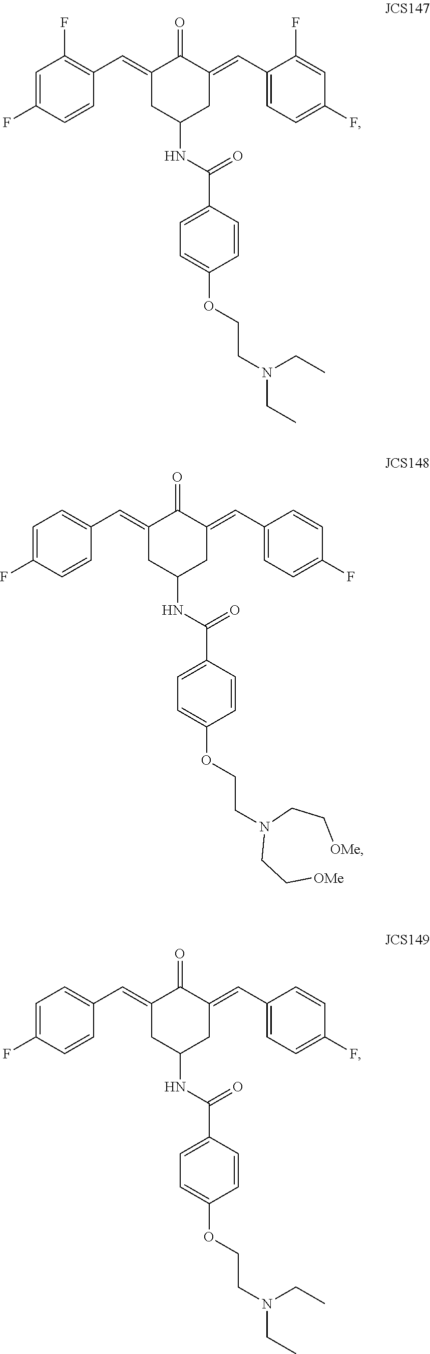 [SMILES: CCN(CC)CCOc1ccc(C(=O)NC2C/C(=C\c3ccc(F)cc3F)C(=O)/C(=C/c3ccc(F)cc3F)C2)cc1 ; COCCN(CCOC)CCOc1ccc(C(=O)NC2C/C(=C\c3ccc(F)cc3)C(=O)/C(=C/c3ccc(F)cc3)C2)cc1 ; CCN(CC)CCOc1ccc(C(=O)NC2C/C(=C\c3ccc(F)cc3)C(=O)/C(=C/c3ccc(F)cc3)C2)cc1] 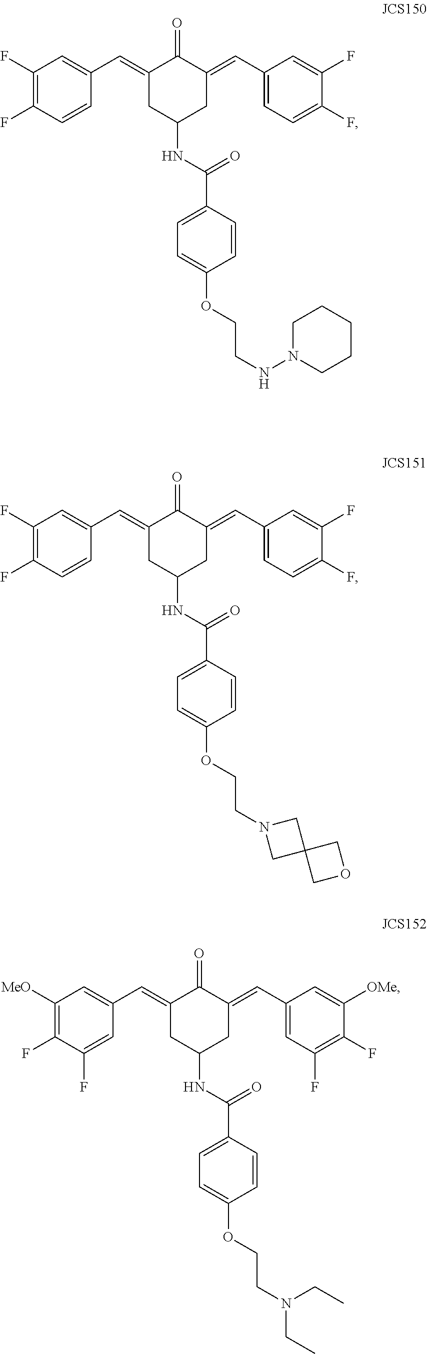 [SMILES: O=C1/C(=C/c2ccc(F)c(F)c2)CC(NC(=O)c2ccc(OCCNN3CCCCC3)cc2)C/C1=C\c1ccc(F)c(F)c1 ; O=C1/C(=C/c2ccc(F)c(F)c2)CC(NC(=O)c2ccc(OCCN3CC4(COC4)C3)cc2)C/C1=C\c1ccc(F)c(F)c1 ; CCN(CC)CCOc1ccc(C(=O)NC2C/C(=C\c3cc(F)c(F)c(OC)c3)C(=O)/C(=C/c3cc(F)c(F)c(OC)c3)C2)cc1] 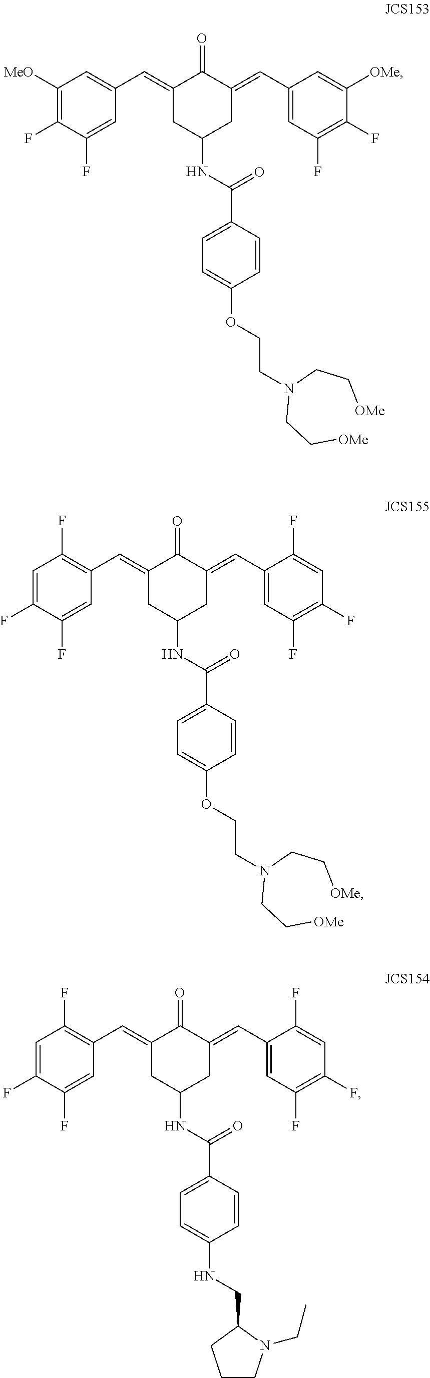 [SMILES: COCCN(CCOC)CCOc1ccc(C(=O)NC2C/C(=C\c3cc(F)c(F)c(OC)c3)C(=O)/C(=C/c3cc(F)c(F)c(OC)c3)C2)cc1 ; COCCN(CCOC)CCOc1ccc(C(=O)NC2C/C(=C\c3cc(F)c(F)cc3F)C(=O)/C(=C/c3cc(F)c(F)cc3F)C2)cc1 ; CCN1CCC[C@H]1CNc1ccc(C(=O)NC2C/C(=C\c3cc(F)c(F)cc3F)C(=O)/C(=C/c3cc(F)c(F)cc3F)C2)cc1] 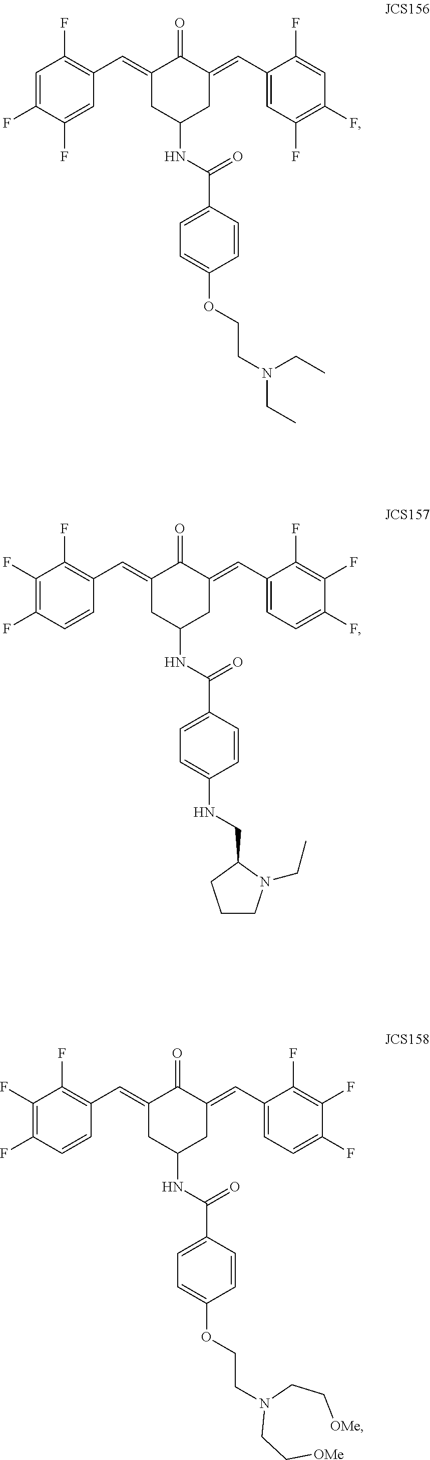 [SMILES: CCN(CC)CCOc1ccc(C(=O)NC2C/C(=C\c3cc(F)c(F)cc3F)C(=O)/C(=C/c3cc(F)c(F)cc3F)C2)cc1 ; CCN1CCC[C@H]1CNc1ccc(C(=O)NC2C/C(=C\c3ccc(F)c(F)c3F)C(=O)/C(=C/c3ccc(F)c(F)c3F)C2)cc1 ; COCCN(CCOC)CCOc1ccc(C(=O)NC2C/C(=C\c3ccc(F)c(F)c3F)C(=O)/C(=C/c3ccc(F)c(F)c3F)C2)cc1] 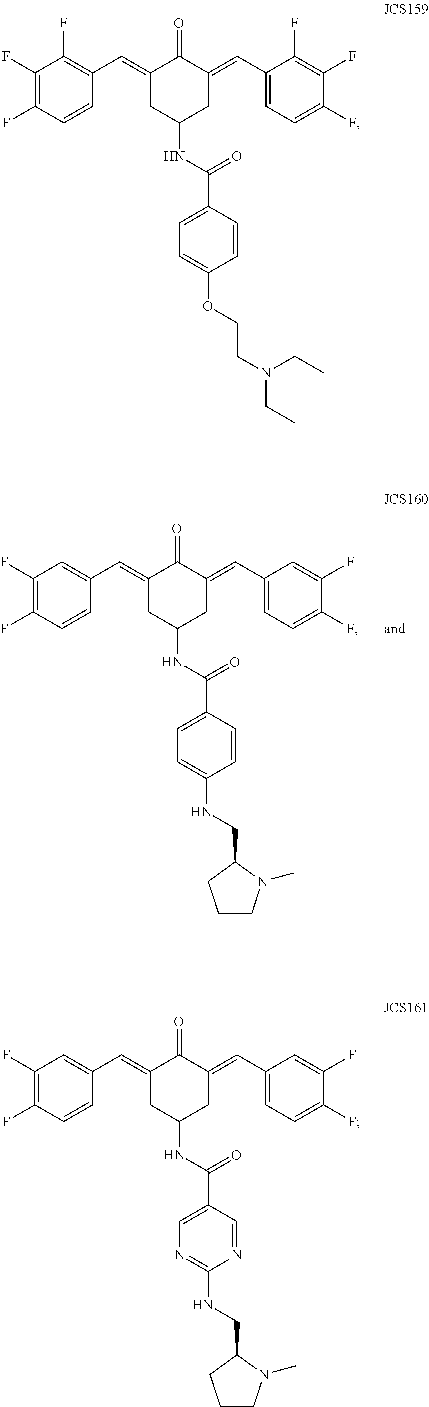 [SMILES: CCN(CC)CCOc1ccc(C(=O)NC2C/C(=C\c3ccc(F)c(F)c3F)C(=O)/C(=C/c3ccc(F)c(F)c3F)C2)cc1 ; CN1CCC[C@H]1CNc1ccc(C(=O)NC2C/C(=C\c3ccc(F)c(F)c3)C(=O)/C(=C/c3ccc(F)c(F)c3)C2)cc1 ; CN1CCC[C@H]1CNc1ncc(C(=O)NC2C/C(=C\c3ccc(F)c(F)c3)C(=O)/C(=C/c3ccc(F)c(F)c3)C2)cn1]
or a pharmaceutically acceptable salt thereof.

156. A pharmaceutical composition comprising the compound of any one of claims 1-155 and a pharmaceutically acceptable excipient.

157. A method of treating a cancer in a subject in need thereof, comprising administering to the subject an amount of a compound of any one claims 1-155 or a pharmaceutically acceptable salt thereof.

158. The method of claim 157, wherein the cancer is breast cancer, head and neck cancer, lung cancer, prostate cancer, or ovarian cancer.

159. The method of claim 157, wherein the cancer is testicular cancer, cervical cancer, bladder cancer, esophageal cancer, mesothelioma, or brain cancer (e.g., neuroblastoma).

160. The method of any one of claims 157-159, wherein the cancer is relapsed.

161. The method of any one of claims 157-160, wherein the cancer is refractory.

162. The method of any one of claims 157-161, wherein the cancer is resistant to treatment with olaparib.

163. The method of any one of claims 157-162, wherein the cancer is resistant to treatment with cisplatin.

164. A method of inhibiting repair of DNA in a subject in need thereof, comprising administering to the subject an amount of a compound of any one claims 1-155 or a pharmaceutically acceptable salt thereof.

165. A method of inhibiting PARP and ATR in a subject in need thereof, comprising administering to the subject an amount of a compound of any one claims 1-155 or a pharmaceutically acceptable salt thereof.
